(19) United States
(12) Patent Application Publication
(10) Pub. No.: US 2025/0051317 A1
(43) Pub. Date: Feb. 13, 2025

(54) NITROGEN-CONTAINING COMPOUND, ORGANIC ELECTROLUMINESCENT DEVICE, AND ELECTRONIC APPARATUS

(71) Applicant: SHAANXI LIGHTE OPTOELECTRONICS MATERIAL CO., LTD., High-tech Zone Xi'an, Shaanxi (CN)

(72) Inventors: Xianbin XU, Beilin District Xi'an, Shaanxi (CN); Lei YANG, Chang'an District Xi'an, Shaanxi (CN)

(21) Appl. No.: 18/718,395

(22) PCT Filed: May 31, 2023
(86) PCT No.: PCT/CN2023/097643

§ 371 Date: Jun. 10, 2024

(30) Foreign Application Priority Data
Sep. 22, 2022 (CN) 202211157559.6

(51) Int. Cl. C07D 413/04; C07B 59/00; C07D 263/56; C07D 413/14; C07D 417/10; C07D 417/14; H10K 85/60

(52) U.S. Cl. CPC C07D 413/04; C07B 59/004; C07D 263/56; C07D 413/14; C07D 417/10; C07D 417/14; H10K 85/622; H10K 85/633; H10K 85/654; H10K 85/656; H10K 85/6572; H10K 85/6574; H10K 85/6576; C07B 2200/05

(57) ABSTRACT

The present application relates to the technical field of organic electroluminescent materials, and provides a nitrogen-containing compound, an organic electroluminescent device comprising same, and an electronic apparatus. The nitrogen-containing compound of the present application comprises a parent nucleus structure of a benzophenoxazole/thiazole group. When the nitrogen-containing compound is used as a host material for an emission layer of the organic electroluminescent device, the luminous efficiency and service life of the device can be remarkably improved.

### CROSS-REFERENCE TO RELATED APPLICATIONS

This present disclosure claims the priority of Chinese patent application No. CN202211157559.6 filed on Sep. 22, 2022, which is incorporated herein by reference in its entirety as a part of this application.

### FIELD OF THE INVENTION

The present application relates to the technical field of organic electroluminescent materials, and especially relates to a nitrogen-containing compound, and an organic electroluminescent device comprising same, and an electronic apparatus.

### BACKGROUND OF THE INVENTION

With the development of electronic technology and the progress of material science, the application range of electronic components used to realize electroluminescence or photoelectric conversion is more and more extensive. Organic electroluminescent devices (OLED) usually comprise a cathode and an anode disposed opposite to each other, and a functional layer disposed between the cathode and the anode. The functional layer is composed of multiple organic or inorganic film layers, and generally comprises an organic light-emitting layer, a hole transport layer, an electron transport layer, etc. When a voltage is applied to the anode and cathode, the two electrodes generate an electric field. Under a influence of the electric field, electrons on the cathode side move to the electroluminescent layer, and holes on the anode side also move to the luminescent layer. The electrons and holes combine to form excitons in the electroluminescent layer, and the excitons are in an excited state to release energy to the outside, so that the electroluminescent layer emits light to the outside.

In the existing organic electroluminescent devices, the most important problem is the lifetime and efficiency. With the large-scale display, the driving voltage is also increased, and the luminous efficiency and current efficiency need to be improved. Therefore, it is necessary to continue to develop new materials to further improve the performance of organic electroluminescent devices.

### SUMMARY OF THE INVENTION

Directed against the above problems with the existing technology, an objective of the present disclosure is to provide a nitrogen-containing compound, and an electronic device and an electronic apparatus comprising the nitrogen-containing compound. The nitrogen-containing compound, when used in organic electroluminescent devices, can improve the performance of electroluminescent devices.

According to a first aspect of the present disclosure, there is provided a nitrogen-containing compound having a structure shown in Formula 1:

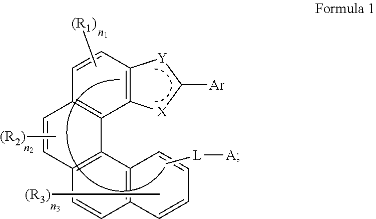

wherein,
group A is selected from Formula A-1 or A-2;

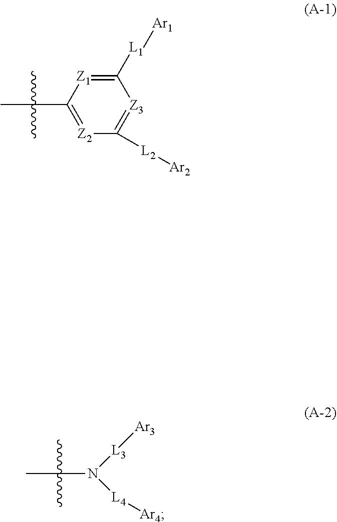

one of X and Y is —N═, and the other is O or S;
Z₁ and Z₃ are N, and Z₂ is selected from C(H) or N; or Z₁ and Z₂ are N, and Z₃ is selected from C(H) or N;
L, L₁, L₂, L₃, and L₄ are identical or different, and are each independently selected from a single bond, a substituted or unsubstituted arylene having 6 to 30 carbon atoms, and a substituted or unsubstituted heteroarylene having 3 to 30 carbon atoms;
Ar, Ar₁, Ar₂, Ar₃, and Ar₄ are identical or different, and are each independently selected from a substituted or unsubstituted aryl having 6 to 40 carbon atoms, and a substituted or unsubstituted heteroaryl having 3 to 40 carbon atoms;
substituent(s) in L, L₁, L₂, L₃, L₄, Ar, Ar₁, Ar₂, Ar₃, and Ar₄ are identical or different, and are each independently selected from deuterium, cyano, a halogen, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl group, an aryl having 6 to 20 carbon atoms, a deuterated aryl having 6 to 20 carbon atoms, a heteroaryl having 3 to 20 carbon atoms, a cycloalkyl having 3 to 10 carbon atoms, an alkoxy having 1 to 10 carbon atoms, an alkylthio having 1 to 10 carbon atoms, an aryloxy having 6 to 20 carbon atoms, and an arylthio having 6 to 20 carbon atoms; optionally, any two adjacent substituents form a ring;
R₁, R₂ and R₃ are identical or different, and are each independently selected from hydrogen, deuterium, cyano, a halogen, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl group, an aryl having 6 to 20 carbon atoms, a heteroaryl having 3 to 20 carbon atoms or a cycloalkyl having 3 to 10 carbon atoms; n₁ and n₂ are each independently selected from 0, 1, and 2, and n₃ is selected from 0, 1, 2, 3, 4, 5, and 6.

According to a second aspect of the present disclosure, an organic electroluminescent devices is provided. The organic electroluminescent device comprises an anode and a cathode disposed opposite to each other, and a functional layer disposed between the anode and the cathode. The functional layer comprises the nitrogen-containing compound described above.

According to a third aspect of the present disclosure, an electronic device is provided. The electronic device comprises the organic electroluminescent device described in the second aspect.

The structure of the compounds in the present disclosure comprises a benzophenoxazole or thiazole having a large conjugated system and a strong intermolecular force, which can improve the carrier mobility of the compound. The parent nucleus is connected with a triazine or pyrimidine group and an aromatic amine group respectively, and when it is used as an electron transport type host material and a hole transport type host material respectively, the balance of carriers in the luminescent layer can be improved, the carrier recombination region can be widened, the exciton generation and utilization efficiency can be improved, and the luminous efficiency and the service life of the device can be increased.

### BRIEF DESCRIPTION OF THE DRAWINGS

The brief of the drawings are provided to provide a further understanding of this present disclosure and constitute a part of the specification, and together with the following detailed description, serve to explain this present disclosure, but do not constitute a limitation of this present disclosure.

FIG. 1 is a schematic structural diagram of an organic electroluminescent device according to an embodiment of the present disclosure.

FIG. 2 is a schematic structural diagram of an electronic device according to an embodiment of the present disclosure.

### LIST OF REFERENCE SIGNS

100: anode; 200: cathode; 300: functional layer; 310: hole injection layer; 321: first hole transport layer; 322: second hole transport layer; 330: organic light-emitting layer; 340: electron transport layer; 350: electron injection layer; 400: electronic apparatus.

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present disclosure more comprehensive and complete, and to communicate the concepts of these exemplary embodiments fully to those of ordinary skill in the art. Features, structures, or characteristics described herein can be combined in one or more embodiments in any suitable manner. In the following description, many specific details are provided to give a full understanding of the embodiments of the present disclosure.

In a first aspect, the present disclosure provides a nitrogen-containing compound having a structure shown in Formula 1:

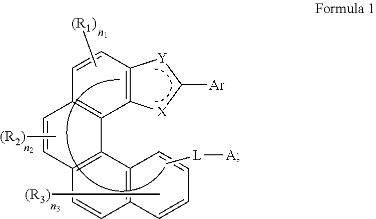

wherein, group A is selected from Formula A-1 or A-2;

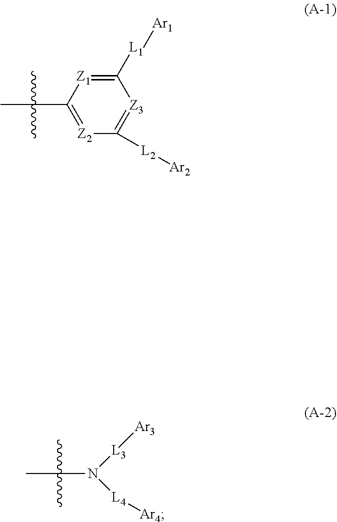

one of X and Y is —N═, and the other is O or S;
Z₁ and Z₃ are N, and Z₂ is selected from C(H) or N; or Z₁ and Z₂ are N, and Z₃ is selected from C(H) or N;
L, L₁, L₂, L₃, and L₄ are identical or different, and are each independently selected from a single bond, a substituted or unsubstituted arylene having 6 to 30 carbon atoms, and a substituted or unsubstituted heteroarylene having 3 to 30 carbon atoms;
Ar, Ar₁, Ar₂, Ar₃, and Ar₄ are identical or different, and are each independently selected from a substituted or unsubstituted aryl having 6 to 40 carbon atoms, and a substituted or unsubstituted heteroaryl having 3 to 40 carbon atoms;
substituent(s) in L, L₁, L₂, L₃, L₄, Ar, Ar₁, Ar₂, Ar₃, and Ar₄ are identical or different, and are each independently selected from deuterium, cyano, a halogen, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, triphenylsilyl group, an aryl having 6 to 20 carbon atoms, a deuterated aryl having 6 to 20 carbon atoms, a heteroaryl having 3 to 20 carbon atoms, a cycloalkyl having 3 to 10 carbon atoms, an alkoxy having 1 to 10 carbon atoms, an alkylthio having 1 to 10 carbon atoms, an aryloxy having 6 to 20 carbon atoms, and an arylthio having 6 to 20 carbon atoms; optionally, any two adjacent substituents form a ring;
R₁, R₂ and R₃ are identical or different, and are each independently selected from hydrogen, deuterium, cyano, a halogen, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, triphenylsilyl group, an aryl having 6 to 20 carbon atoms, a heteroaryl having 3 to 20 carbon atoms, and a cycloalkyl having 3 to 10 carbon atoms; n₁ and n₂ are each independently selected from 0, 1, and 2, and n₃ is selected from 0, 1, 2, 3, 4, 5, and 6.

In the present disclosure, the terms “optional” and “optionally” mean that the subsequently described event or environment may or may not occur. For example, “optionally, any two adjacent substituents form a ring” means that these two substituents may or may not form a ring, that is, it includes two adjacent substituents forming a ring and two adjacent substituents not forming a ring. For another example, “optionally, any two adjacent substituents form a ring” means that any two adjacent substituents are connected to each other to form a ring, or any two adjacent substituents can also exist independently. “Any two adjacent substituents” can include two substituents on the same atom, and can also include two adjacent atoms with one substituent respectively; wherein, when there are two substituents on the same atom, the two substituents can form a saturated or unsaturated spiro ring with the atom to which they are connected together; when two adjacent atoms have a substituent respectively, the two substituents can be condensed into a ring.

In the present disclosure, the expression “each . . . independently”may be used interchangeably with the expressions “ . . . each independently” and “ . . . independently”, and all these expressions should be interpreted in a broad sense. They can not only mean that, for same symbols in a different group, the selection of a specific option for one of the symbols and the selection of a specific option for another one of the symbols do not affect each other, but also mean that for same symbols in same groups, the selection of a specific option for one of the symbols and the selection of a specific option for another one of the symbols do not affect each other. Taking

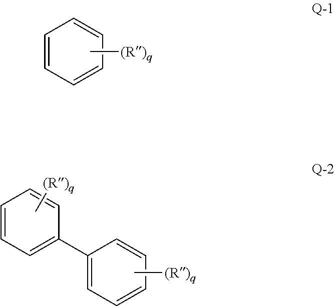

as an example, each q is independently selected from 0, 1, 2 or 3, and each R″ is independently selected from hydrogen, deuterium, fluorine, chlorine, which means: in Formula Q-1, there are q substituents R″ on the benzene ring, wherein each of the substituents R″ may be identical or different, with the selection of an option for one of the substituents R″ and the selection of an option for another one of the substituents R″ not affecting each other; and in Formula Q-2, there are q substituents R″ on each of the two benzene rings of biphenyl, wherein the number q of the substituent R″ on one benzene ring and the number q of the substituent R″ on the other benzene ring may be the same or different, and each substituent R″ may be identical or different, with the selection of an option for one of the substituents R″ and the selection of an option for another one of the substituents R″ not affecting each other.

In the present disclosure, the term “substituted or unsubstituted” means that the functional group defined by the term may or may not have a substituent (hereinafter referred to as Re for ease of description). For example, “substituted or unsubstituted aryl” refers to an aryl group with the substituent Re or an unsubstituted aryl group. The above-mentioned substituents, namely Re, may be, for example, deuterium, halogen, cyano, heteroaryl, aryl, trialkylsilyl, alkyl, haloalkyl, cycloalkyl, etc. The number of substitutions can be one or more.

In the present disclosure, “more” refers to two or more than two, such as two, three, four, five, six, and so on.

The hydrogen atom in the structure of the compounds in the present disclosure includes various isotope atoms of hydrogen, such as hydrogen (H), deuterium (D), or tritium (T).

In the present disclosure, the number of carbon atoms of a substituted or unsubstituted functional group refers to the number of all carbon atoms. For example, if L₁ is a substituted arylene having 12 carbon atoms, the number of all carbon atoms of the arylene and its substituents is 12.

In the present disclosure, aryl refers to any functional group or substituent group derived from an aromatic carbon ring. An aryl group may be a monocyclic aryl group (e.g., phenyl) or a polycyclic aryl group. In other words, an aryl group may be a monocyclic aryl group, a fused cycloaryl group, two or more monocyclic aryl groups linked by carbon-carbon bond conjugation, a monocyclic aryl group and a fused cycloaryl group linked by carbon-carbon bond conjugation, or two or more fused cycloaryl groups linked by carbon-carbon bond conjugation. That is, unless otherwise specified, two or more aromatic groups linked by carbon-carbon bond conjugation may also be regarded as an aryl group in the present disclosure. Among them, fused cycloaryl groups may include, for example, bicyclic fused aryl groups (e.g., naphthyl), tricyclic fused aryl groups (e.g., phenanthryl, fluorenyl, anthryl), etc. An aryl group does not contain a heteroatom such as B, N, O, S, P, Se, and Si, etc. Examples of aryl groups may include, but are not limited to, phenyl, naphthyl, fluorenyl, spiro-difluorenyl, anthryl, phenanthryl, biphenyl, terphenyl, triphenylene, perylenyl, 9,10-benzophenanthryl, pyrenyl, benzofluoranthryl, chrysenyl, etc.

In the present disclosure, “arylene” refers to a divalent or multivalent group formed by further removing one or more hydrogen atom from an aryl group.

In the present disclosure, terphenyl includes

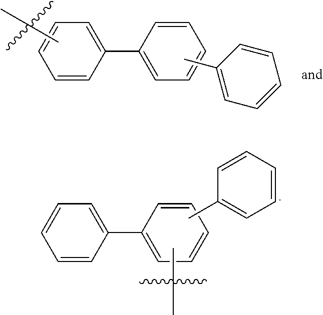

In the present disclosure, the number of carbon atoms of substituted aryl refers to the number of all carbon atoms of aryl and substituted on aryl. For example, a substituted aryl having 18 carbon atoms, refers to the number of all carbon atoms of the aryl and its substituents is 18.

In the present disclosure, the number of carbon atoms of a substituted or unsubstituted aryl (arylene) may be 6, 8, 10, 12, 13, 14, 15, 16, 17, 18, 19, 20, 21, 22, 23, 24, 25, 26, 28, 30, 31, 33, 34, 35, 36, 38, or 40, etc. In some embodiments, a substituted or unsubstituted aryl is a substituted or unsubstituted aryl having 6 to 40 carbon atoms; in other embodiments, a substituted or unsubstituted aryl is a substituted or unsubstituted aryl having 6 to 30 carbon atoms; in other embodiments, substituted or unsubstituted aryl is substituted or unsubstituted aryl having 6 to 25 carbon atoms; in other embodiments, a substituted or unsubstituted aryl is a substituted or unsubstituted aryl having 6 to 15 carbon atoms.

In the present disclosure, fluorenyl can be substituted by one or more substituents. When the above-mentioned fluorenyl is substituted, the substituted fluorenyl may be, but not limited to:

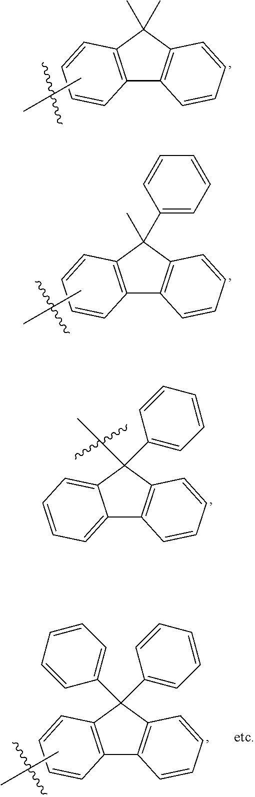

In the present disclosure, an aryl as a substituent of L, L₁, L₂, L₃, L₄, Ar, Ar₁, Ar₂, Ar₃, and Ar₄ is, for example, but not limited to, phenyl, naphthyl, phenanthryl, biphenyl, fluorenyl, dimethylfluorenyl, etc.

In the present disclosure, “heteroaryl” refers to a monovalent aromatic ring containing 1, 2, 3, 4, 5, or 6 heteroatoms or a derivative thereof. The heteroatoms may be one or more selected from B, O, N, P, Si, Se, and S. A heteroaryl group may be a monocyclic heteroaryl group or polycyclic heteroaryl group. In other words, a heteroaryl group may be a single aromatic ring system, or a plurality of aromatic ring systems linked by carbon-carbon bond conjugation, with any of the aromatic ring systems being an aromatic monocyclic ring or a fused aromatic ring. For example, heteroaryl groups may include, but are not limited to, thienyl, furyl, pyrrolyl, imidazolyl, thiazolyl, oxazolyl, oxadiazolyl, triazolyl, pyridyl, dipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, etc, but not limited to thereto.

In the present disclosure, a heteroarylene group is a divalent or multivalent group formed by further removing one or more hydrogen atoms from a heteroaryl group.

In the present disclosure, the number of carbon atoms of a substituted or unsubstituted heteroaryl (heteroarylene) group may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, or 40, etc. In some embodiments, a substituted or unsubstituted heteroaryl group is a substituted or unsubstituted heteroaryl group having 3 to 40 carbon atoms, in other embodiments, a substituted or unsubstituted heteroaryl group is a substituted or unsubstituted heteroaryl group having 3 to 30 carbon atoms, in other embodiments, a substituted or unsubstituted heteroaryl group is a substituted or unsubstituted heteroaryl group having 5 to 12 carbon atoms.

In the present disclosure, a heteroaryl group as a substituent of L, L₁, L₂, L₃, L₄, Ar, Ar₁, Ar₂, Ar₃ and Ar₄ is, for example, but not limited to, pyridyl, carbazolyl, quinolyl, isoquinolyl, phenanthrolinyl, benzoxazolyl, benzothiazolyl, benzimidazolyl, dibenzothienyl, dibenzofuranyl.

In the present disclosure, a substituted heteroaryl may mean that one or more than two hydrogen atoms in the heteroaryl group are substituted by a group such as deuterium, halogen, cyano, aryl, heteroaryl, trialkylsilyl, alkyl, cycloalkyl, haloalkyl, etc. It should be appreciated that the number of carbon atoms of a substituted heteroaryl group is the number of all carbon atoms of the heteroaryl group and substituents in the heteroaryl group.

In the present disclosure, a alkyl having 1 to 10 carbon atoms may include a straight-chain alkyl having 1 to 10 carbon atoms or a branched-chain alkyl having 1 to 10 carbon atoms. The number of carbon atoms of alkyl may specifically be 1, 2, 3, 4, 5, 6, 7, 8, 9, or 10. Specific examples of alkyl groups include, but are not limited to, methyl, ethyl, n-propyl, isopropyl, n-butyl, isobutyl, tert-butyl, n-pentyl, isopentyl, neopentyl, n-hexyl, etc.

In the present disclosure, halogen may be fluorine, chlorine, bromine, or iodine.

In the present disclosure, specific examples of trialkylsilyl group include, but are not limited to, trimethylsilyl, triethylsilyl, etc.

In the present disclosure, specific examples of haloalkyl group include, but are not limited to, trifluoromethyl.

In the present disclosure, the number of a cycloalkyl group having 3 to 10 carbon atoms may be, for example, 3, 4, 5, 6, 7, 8 or 10. Specific examples of cycloalkyl groups include, but are not limited to, cyclopentyl, cyclohexyl, adamantyl.

In the present disclosure, a non-positioned bond is a single bond “ [symbol] ” _extending out from a ring system, and it means that the linkage bond can be linked at one end thereof to any position in the ring system through which the bond penetrates, and linked at the other end thereof to the rest of the compound molecule structure. For example, as shown in Formula (f) below, the naphthalyl group represented by Formula (f) is linked to other positions of the molecule through two non-positional bonds penetrating through the two rings, which indicates any of possible linkages shown in Formulae (f-1) to (f-10):

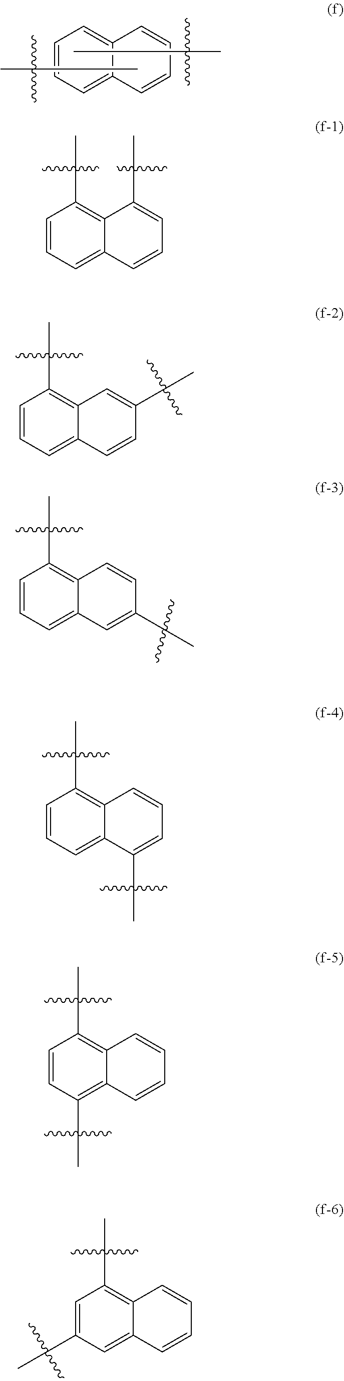

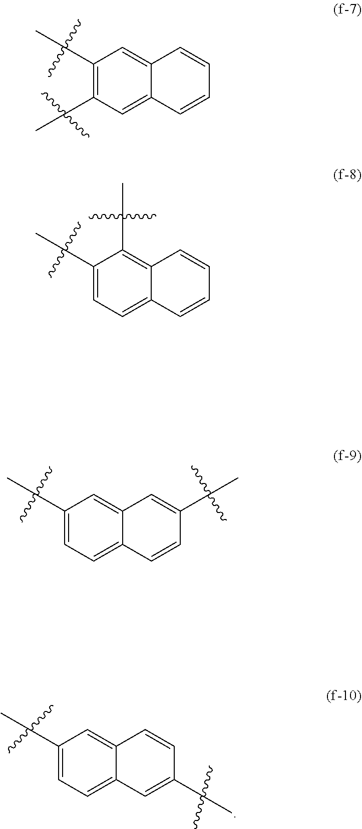 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl group represented by Formula (X′) is linked to other positions of the molecule via a non-positioned connecting bond extending from the middle of a side benzene ring, which indicates any of possible connecting mode shown in Formulae (X′-1) to (X′-4):

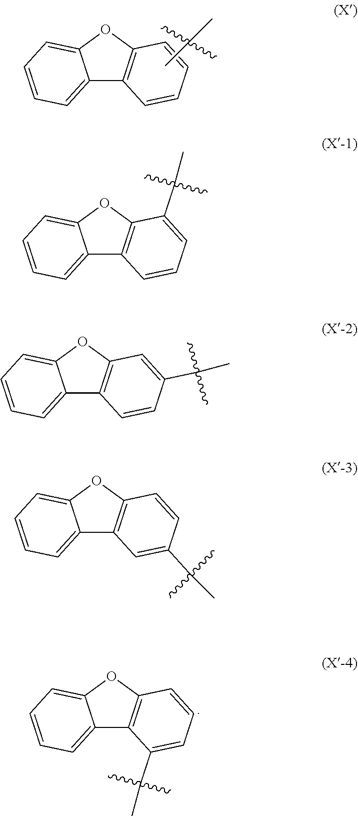

A non-positioned substituent in the present disclosure refers to a substituent linked via single bond extending from the center of a ring system, and it means that the substituent may be linked to any possible position in the ring system. For example, as shown in Formula (Y) below, the substituent R′ represented by Formula (Y) is linked to a quinoline ring via a non-positioned connecting bond, which indicates any of possible connecting mode shown in Formulae (Y-1) to (Y-7):

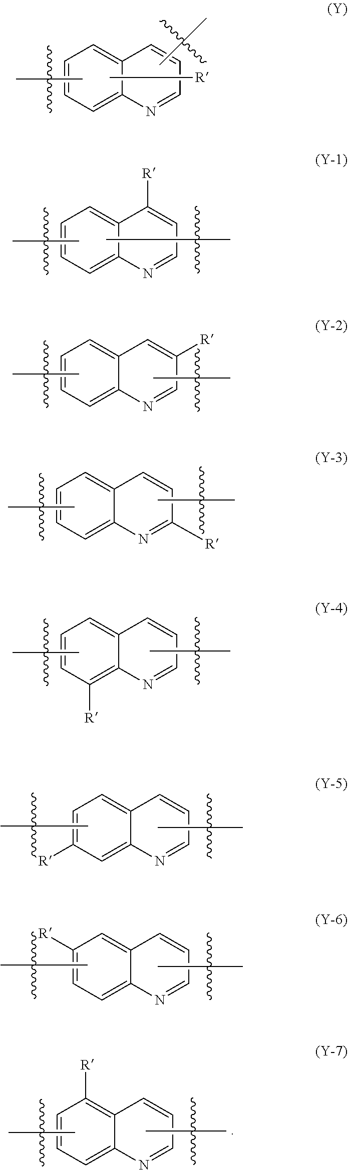

In some embodiments, the compound of Formula 1 is selected from the following structures of Formulae (1-1) to (1-16):

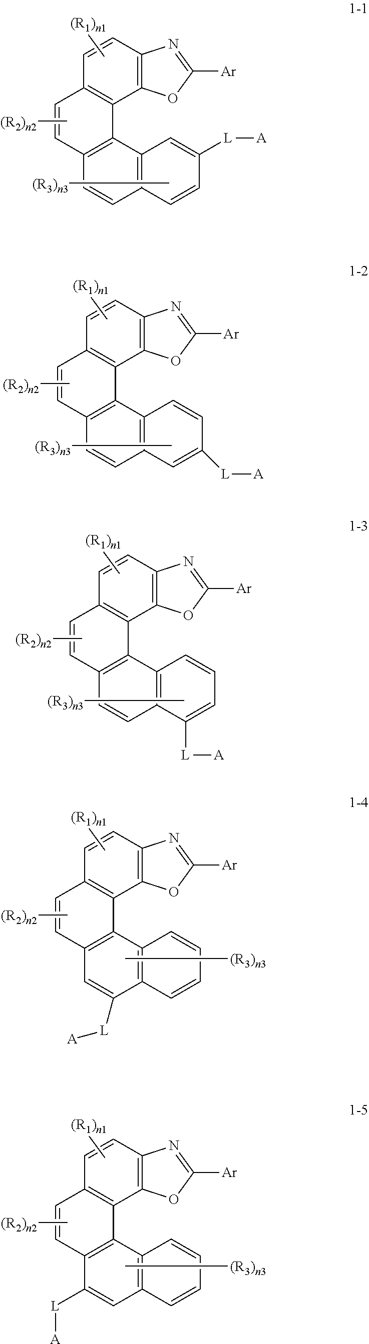

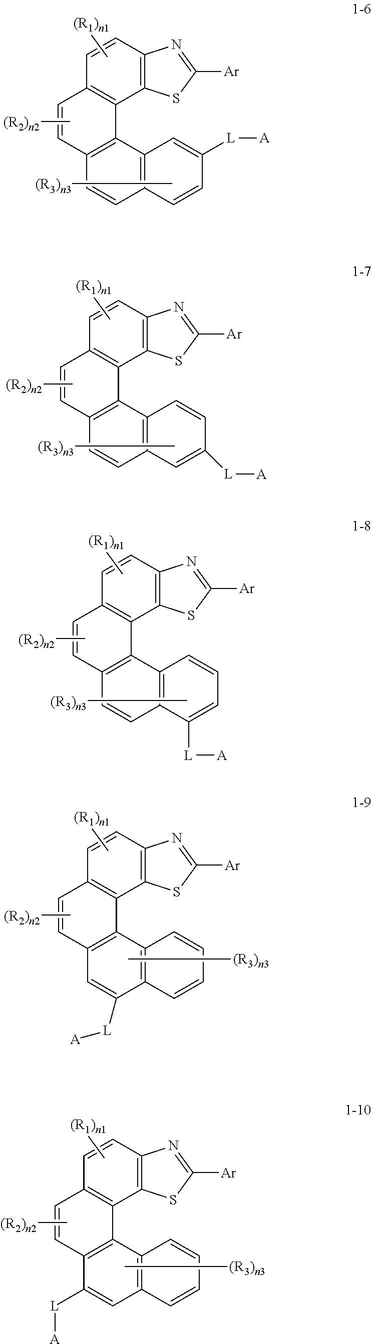

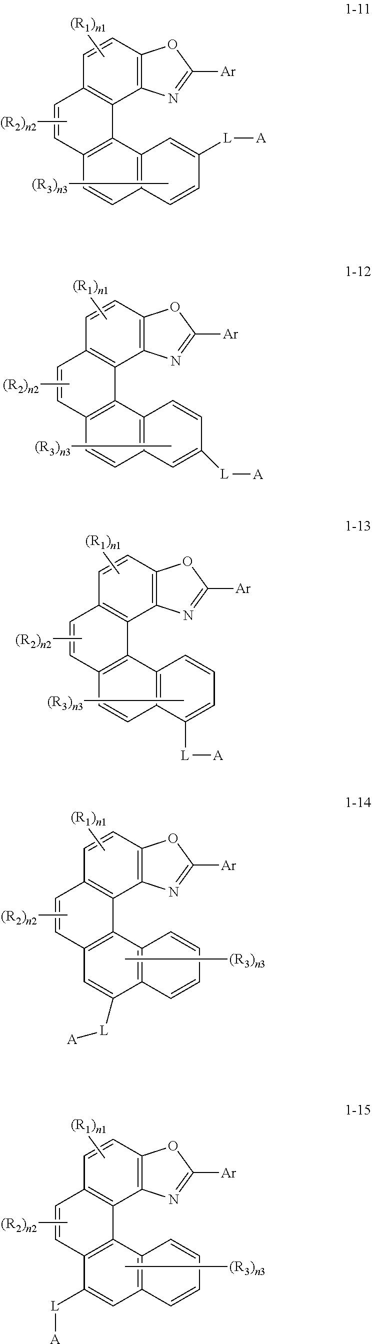

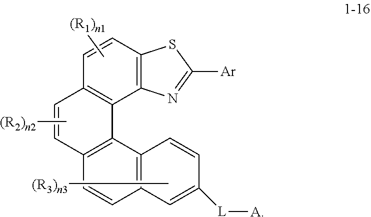

In some embodiments, Z₁, Z₂, and Z₃ are all N.

In some embodiments, Ar, Ar₁, Ar₂, Ar₃ and Ar₄ are identical or different, and are each independently selected from a substituted or unsubstituted aryl having 6, 7, 8, 9, 10, 11, 12, 13, 14, 15, 16, 17, 18, 19, 20, 21, 22, 23, 24 or 25 carbon atoms, and a substituted or unsubstituted heteroaryl having 5, 6, 7, 8, 9, 10, 12, 13, 14, 15, 16, 17, 18, 19, 20, 21, 22, 23, or 24 carbon atoms.

In some embodiments, Ar, Ar₁, Ar₂, Ar₃, and Ar₄ are identical or different, and are each independently selected from a substituted or unsubstituted aryl having 6 to 25 carbon atoms, and a substituted or unsubstituted heteroaryl having 5 to 24 carbon atoms.

In some embodiments, substituents in Ar, Ar₁, Ar₂, Ar₃, and Ar₄ are each independently selected from deuterium, a halogen, cyano, a haloalkyl having 1 to 4 carbon atoms, a deuterated alkyl having 1 to 4 carbon atoms, an alkyl having 1 to 4 carbon atoms, a cycloalkyl having 5 to 10 carbon atoms, an aryl having 6 to 15 carbon atoms, a heteroaryl having 5 to 12 carbon atoms, a trialkylsilyl having 3 to 8 carbon atoms, and a deuterated aryl having 6 to 15 carbon atoms, optionally, any two adjacent substituents form a benzene ring or a fluorene ring.

In some embodiments, Ar, Ar₁, Ar₂, Ar₃, and Ar₄ are each independently selected from the a substituted or unsubstituted phenyl, a substituted or unsubstituted biphenyl, a substituted or unsubstituted terphenyl, a substituted or unsubstituted naphthyl, a substituted or unsubstituted anthryl, a substituted or unsubstituted phenanthryl, a substituted or unsubstituted fluorenyl, a substituted or unsubstituted spirodifluorenyl, a substituted or unsubstituted triphenylene, a substituted or unsubstituted pyrenyl, a substituted or unsubstituted perylenyl, a substituted or unsubstituted pyridyl, a substituted or unsubstituted dibenzothienyl, a substituted or unsubstituted dibenzofuranyl, a substituted or unsubstituted carbazolyl, a substituted or unsubstituted benzothienyl, a substituted or unsubstituted benzoxazolyl, and a substituted or unsubstituted benzimidazolyl.

Optionally, substituent(s) in Ar, Ar₁, Ar₂, Ar₃, and Ar₄ are each independently selected from deuterium, fluorine, cyano, trideuteromethyl, trimethylsilyl, trifluoromethyl, cyclopentyl, cyclohexyl, adamantyl, methyl, ethyl, isopropyl, tert-butyl, phenyl, naphthyl, pyridyl, dibenzofuranyl, dibenzothienyl, and carbazolyl, optionally, any two adjacent substituents form a benzene ring or a fluorene ring.

In some embodiments, Ar, Ar₁, Ar₂, Ar₃, and Ar₄ are each independently selected from a substituted or unsubstituted group W; the unsubstituted group W is selected from the group consisting of the following groups:

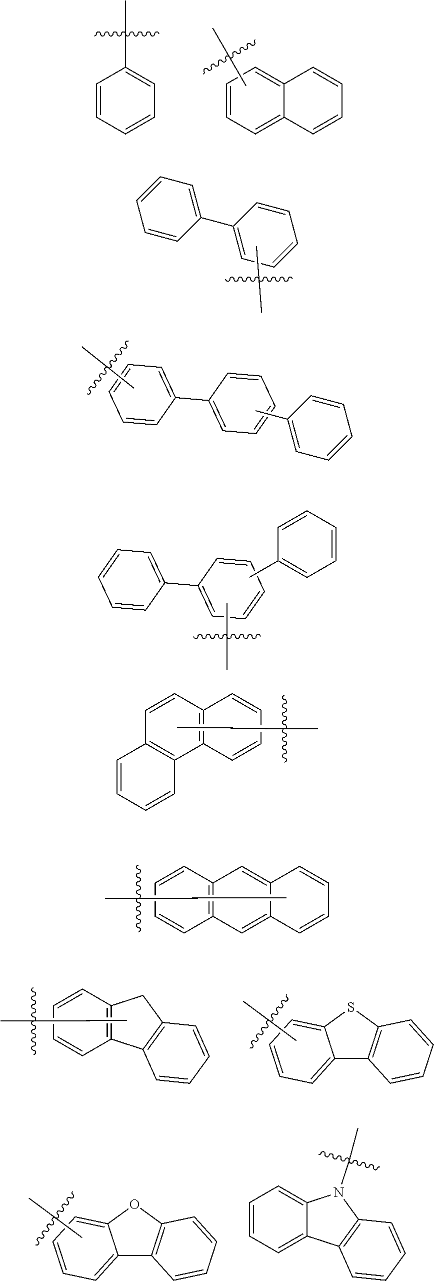

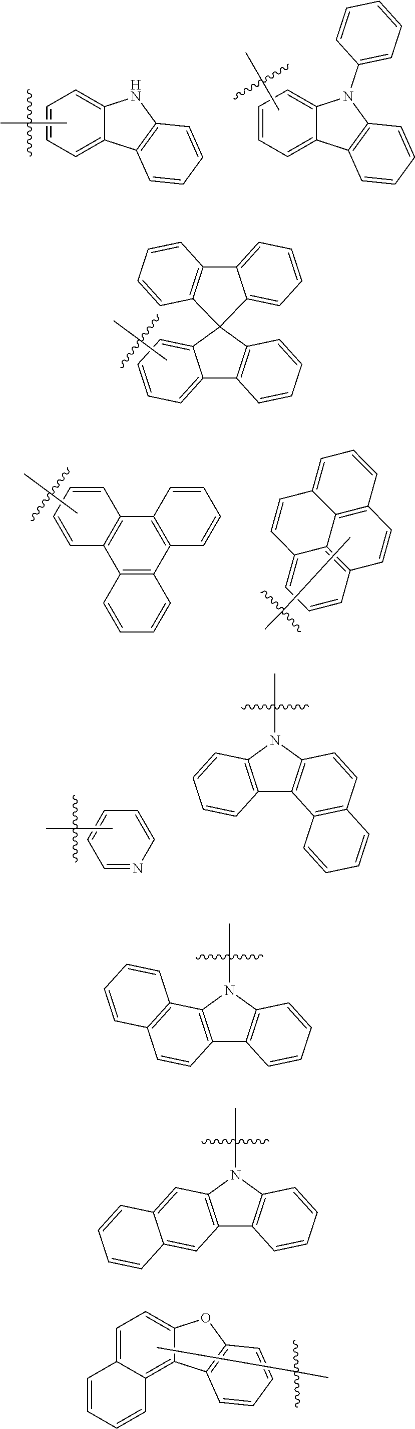

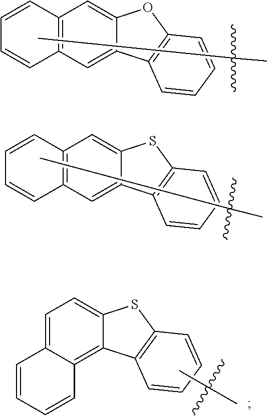

the substituted group W has one or more substituents, and the substituent in substituted group W is independently selected from deuterium, fluorine, cyano, trideuteromethyl, trimethylsilyl, trifluoromethyl, cyclopentyl, cyclohexyl, adamantyl, methyl, ethyl, isopropyl, tert-butyl, phenyl, naphthyl, pyridyl, dibenzofuranyl, dibenzothienyl and carbazolyl, and when the number of the substituents in group W is greater than 1, the substituents are identical or different.

In some embodiments, Ar₁, Ar₂, Ar₃, and Ar₄ are identical or different, and are each independently selected from the group consisting of the following groups:

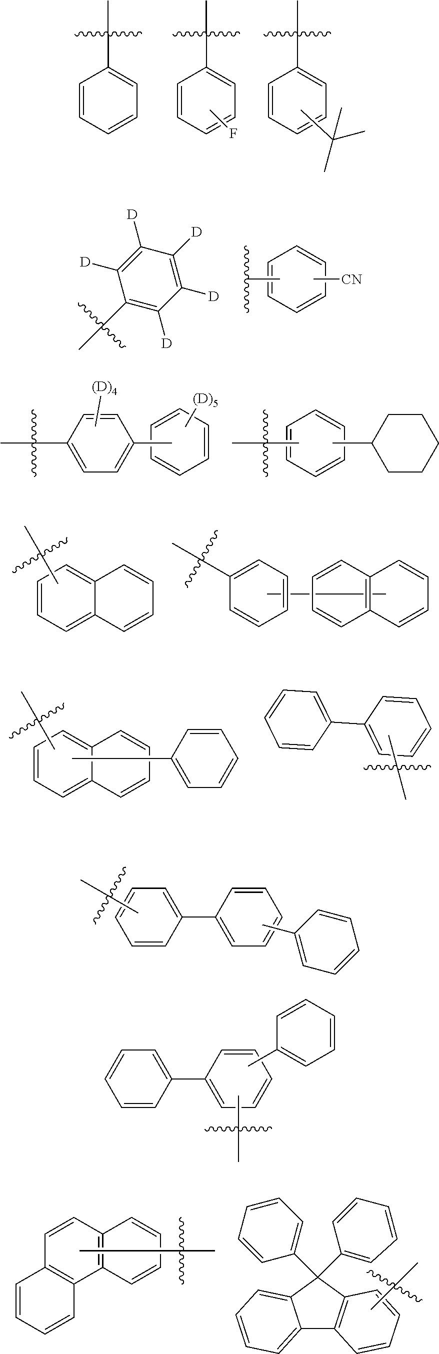

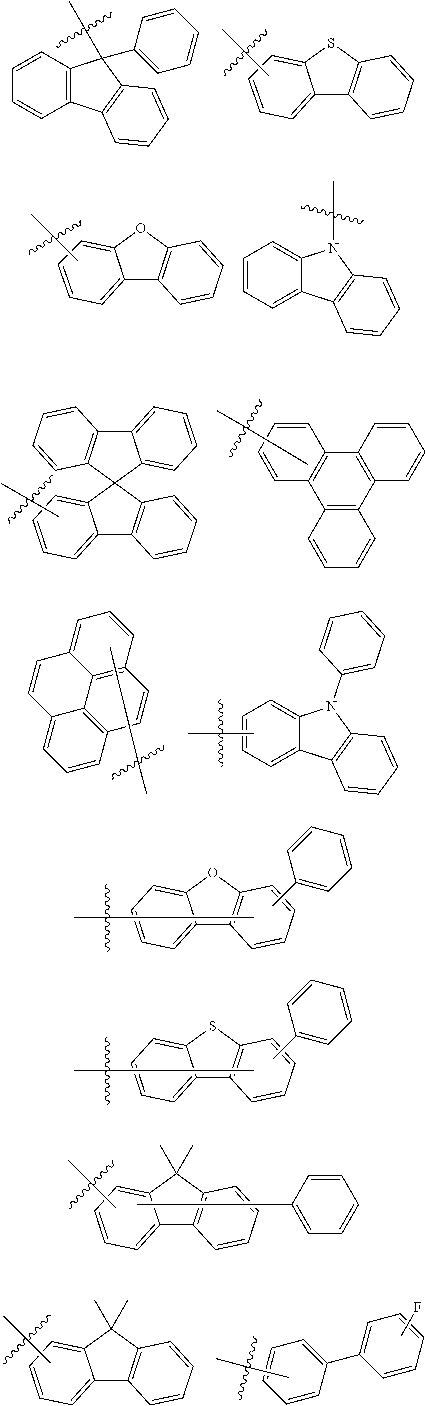

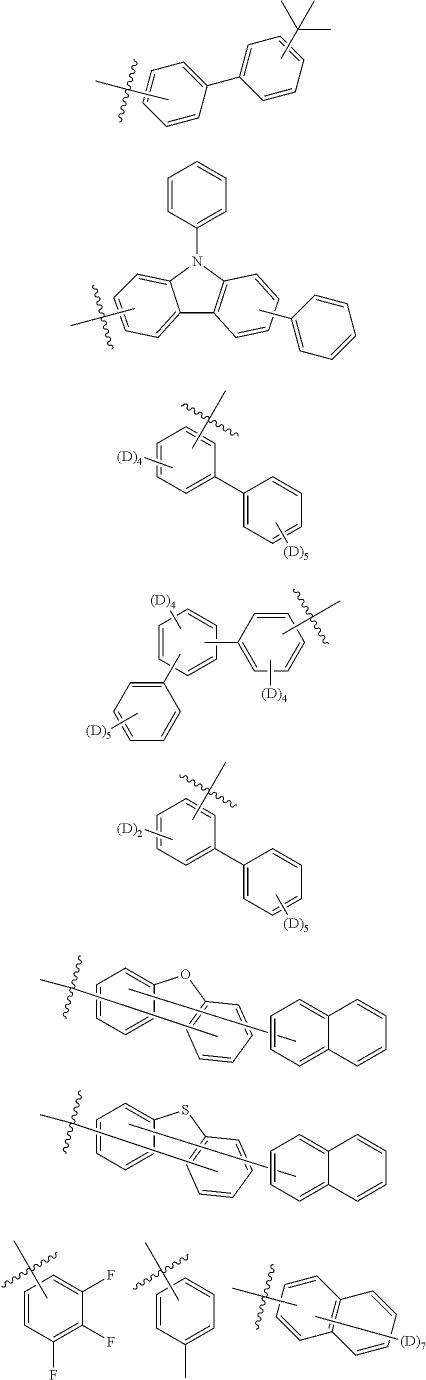

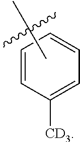

In some embodiments, Ar, Ar₁, Ar₂, Ar₃ and Ar₄ are identical or different, and are each independently selected from the group consisting of the following groups:

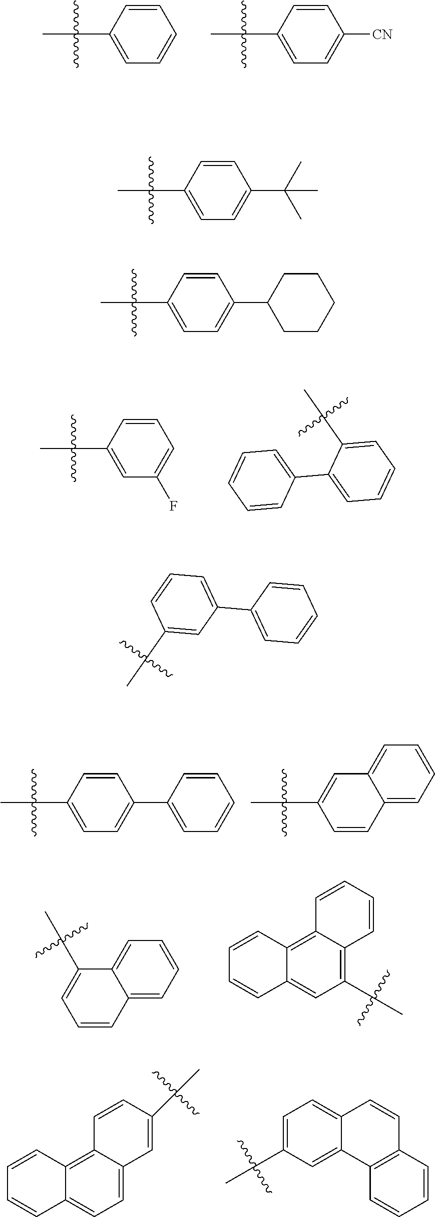

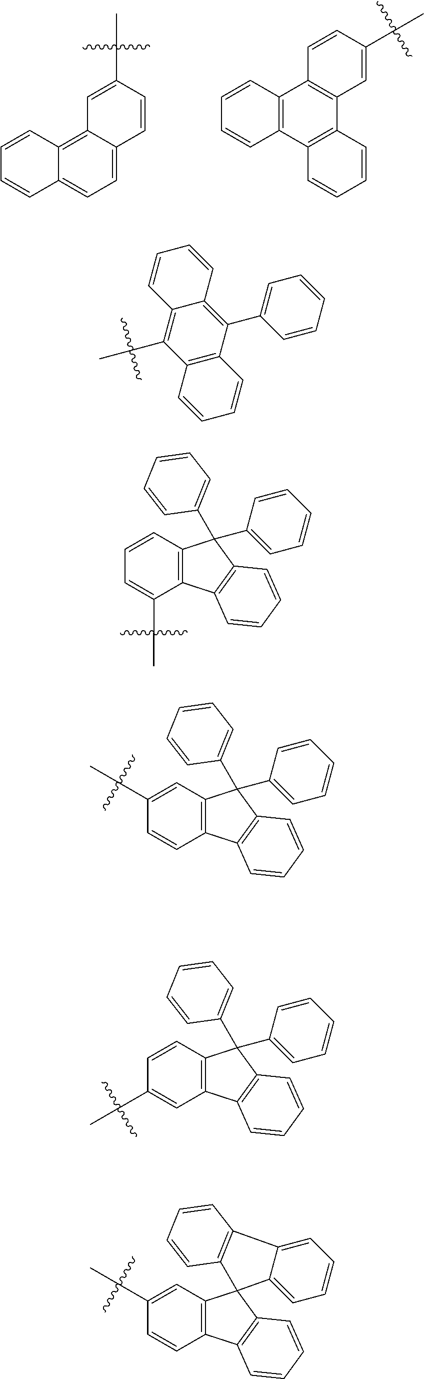

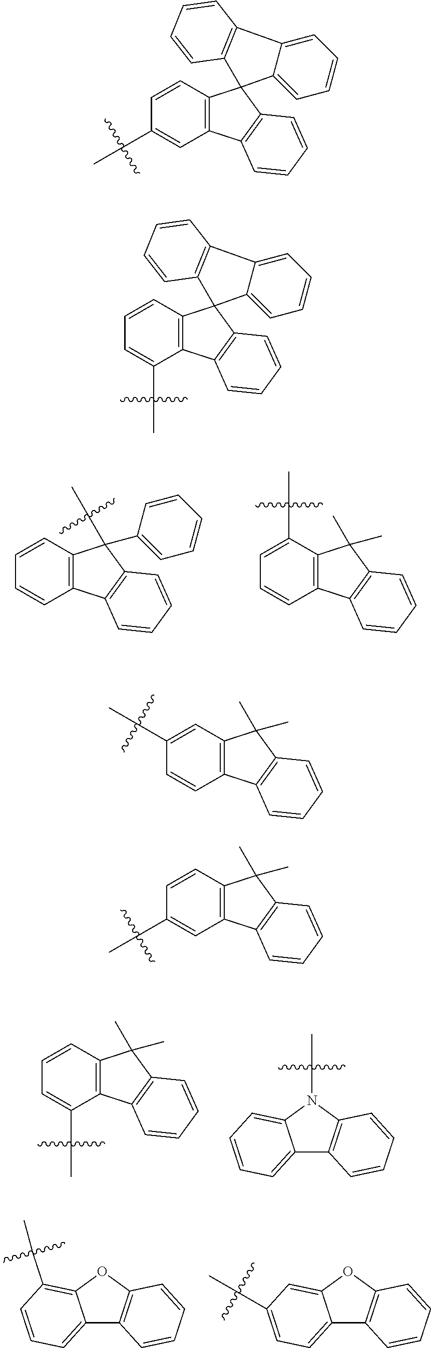

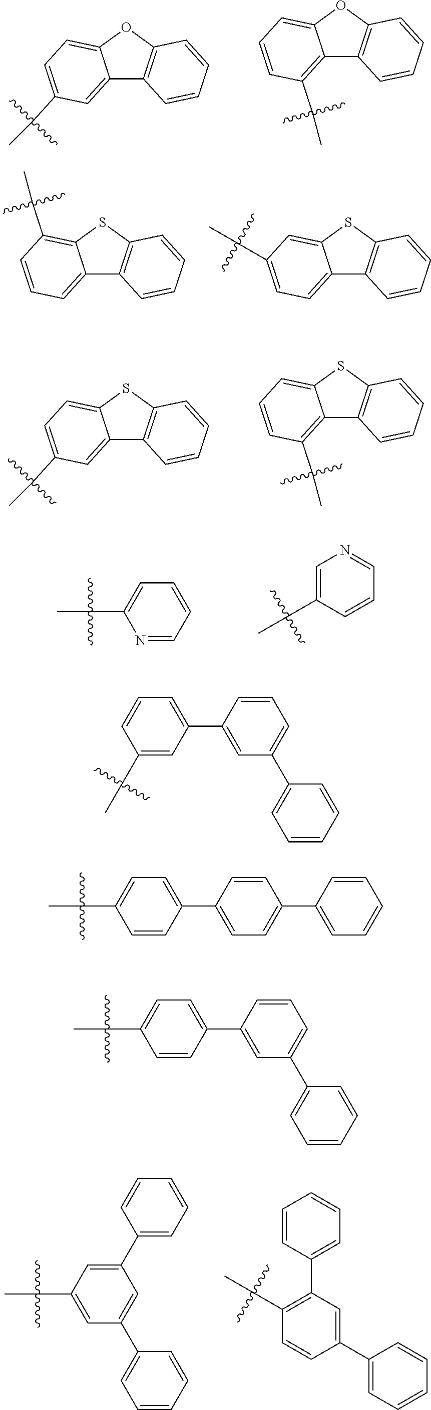

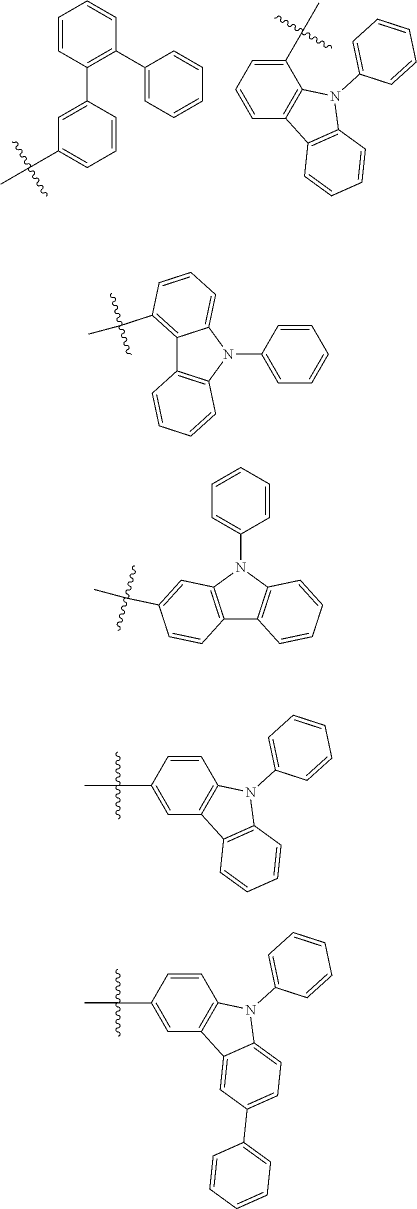

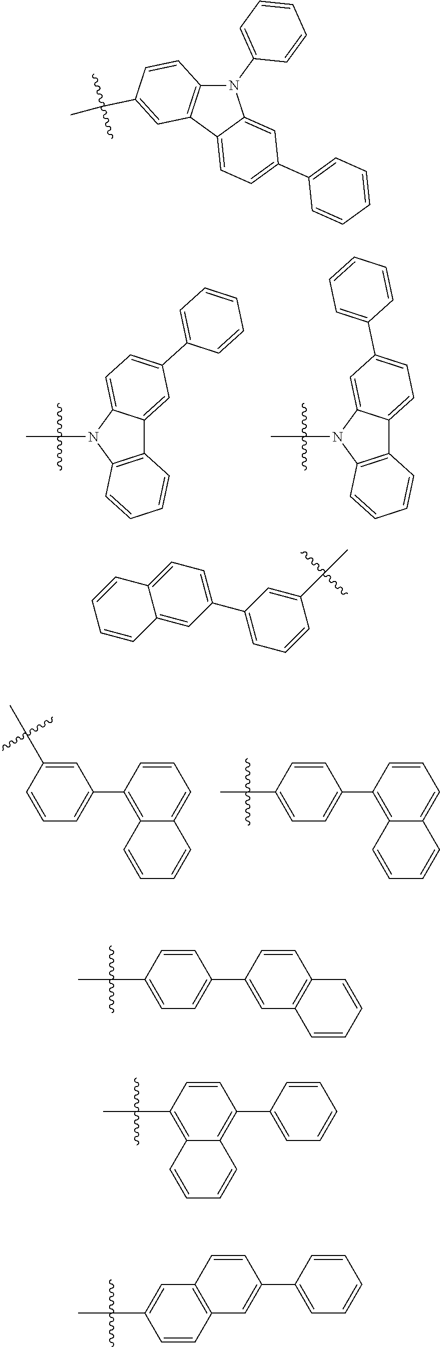

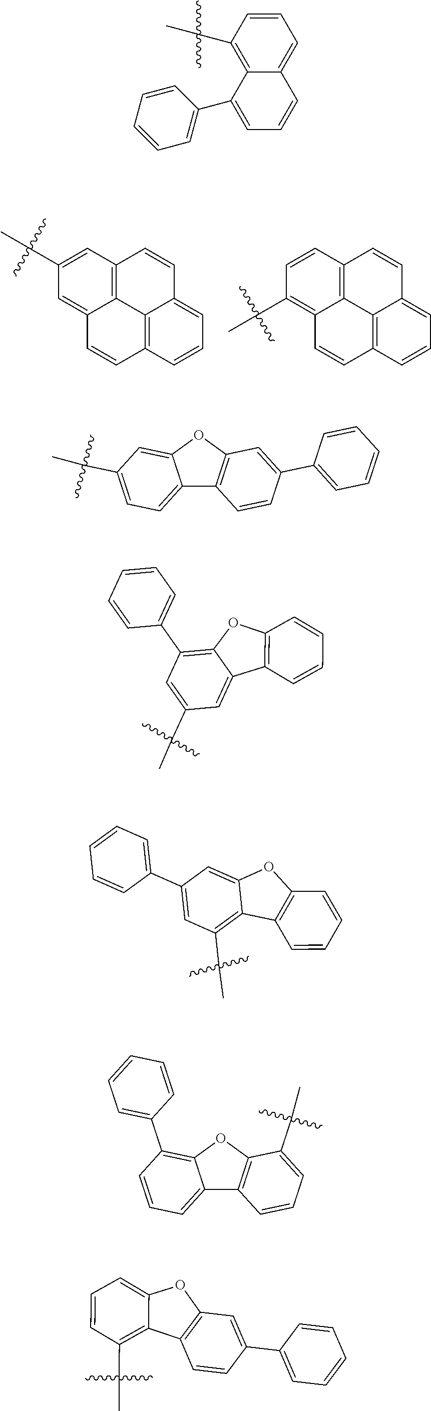

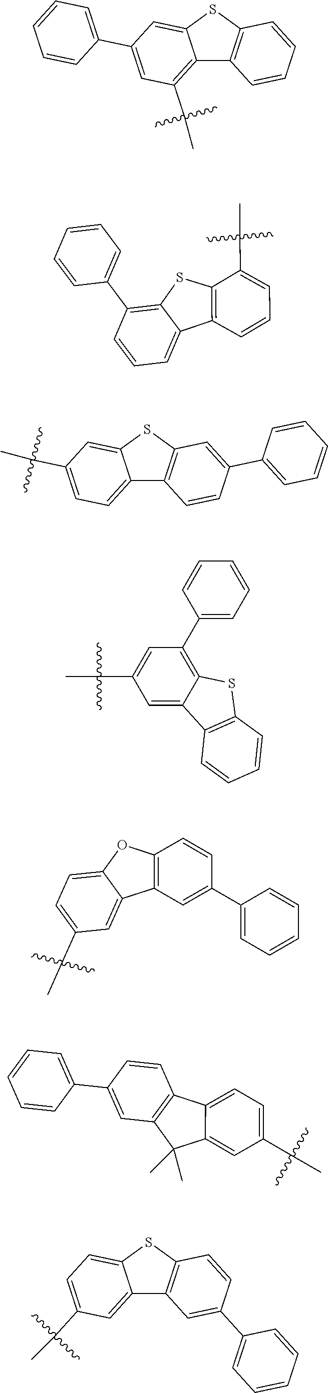

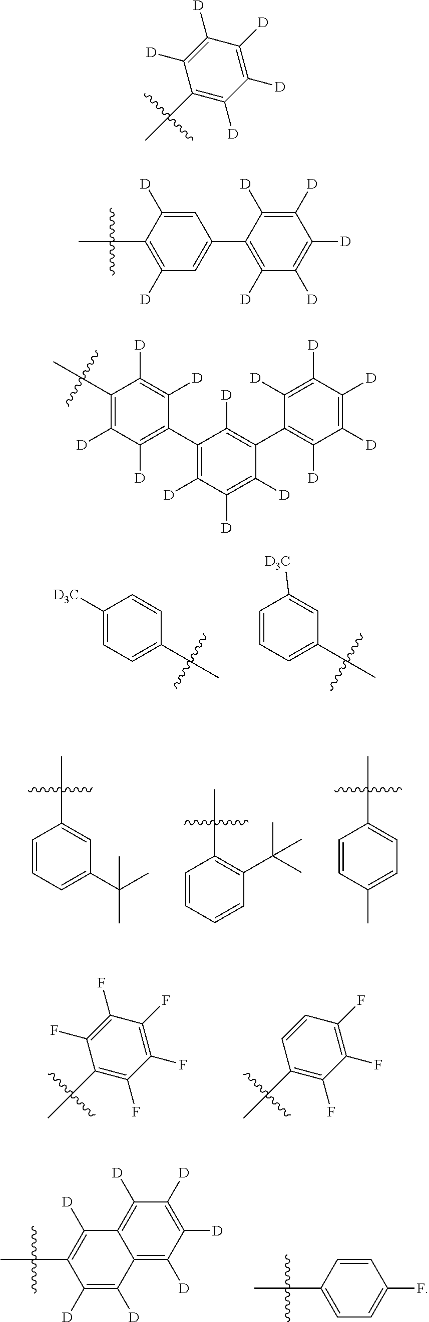

In some embodiments, Ar is selected from a substituted or unsubstituted aryl having 6 to 18 carbon atoms, and a substituted or unsubstituted heteroaryl having 12 to 18 carbon atoms.

In some embodiments, Ar is selected from a substituted or unsubstituted phenyl, a substituted or unsubstituted biphenyl, a substituted or unsubstituted terphenyl, a substituted or unsubstituted naphthyl, a substituted or unsubstituted phenanthryl, a substituted or unsubstituted fluorenyl, a substituted or unsubstituted triphenylene, a substituted or unsubstituted dibenzothienyl, a substituted or unsubstituted dibenzofuranyl, and a substituted or unsubstituted carbazolyl.

Optionally, substituent(s) in Ar are independently selected from deuterium, fluorine, cyano, trideuteromethyl, trimethylsilyl, trifluoromethyl, methyl, ethyl, isopropyl, tert-butyl, phenyl, naphthyl, and deuterophenyl.

In some embodiments, Ar is selected from the group consisting of the following groups:

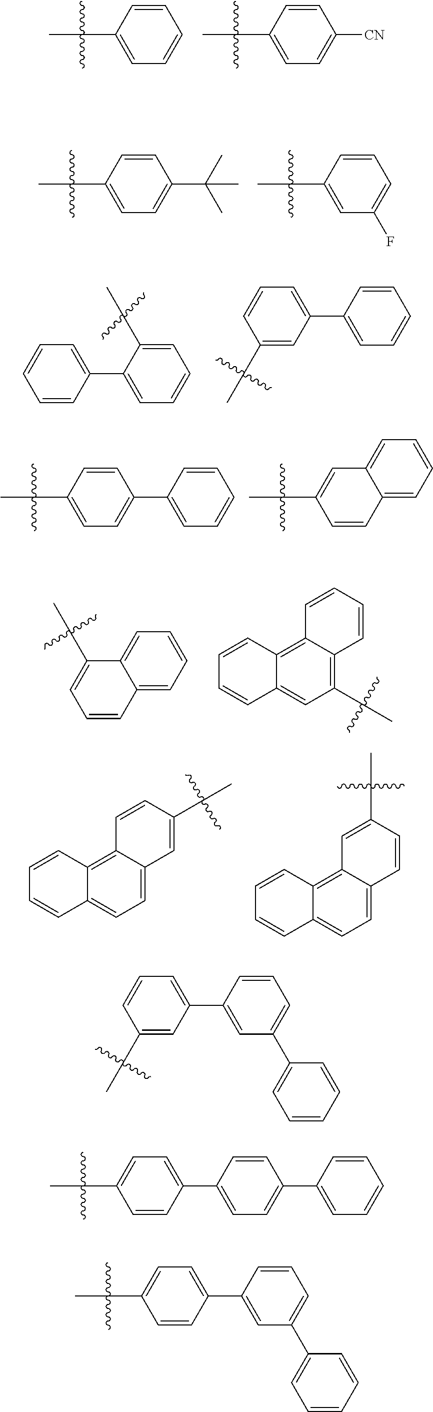

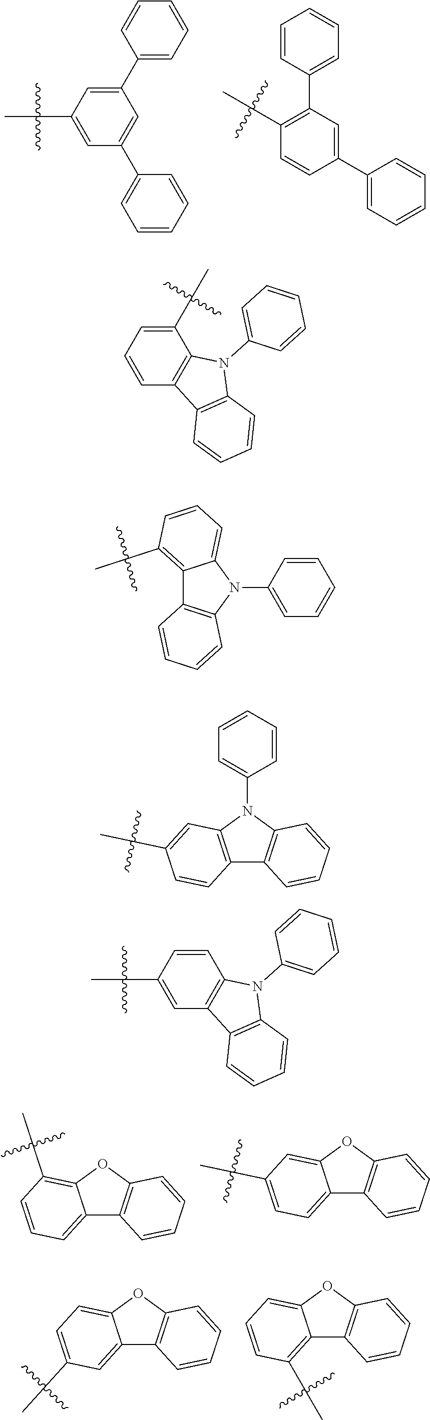

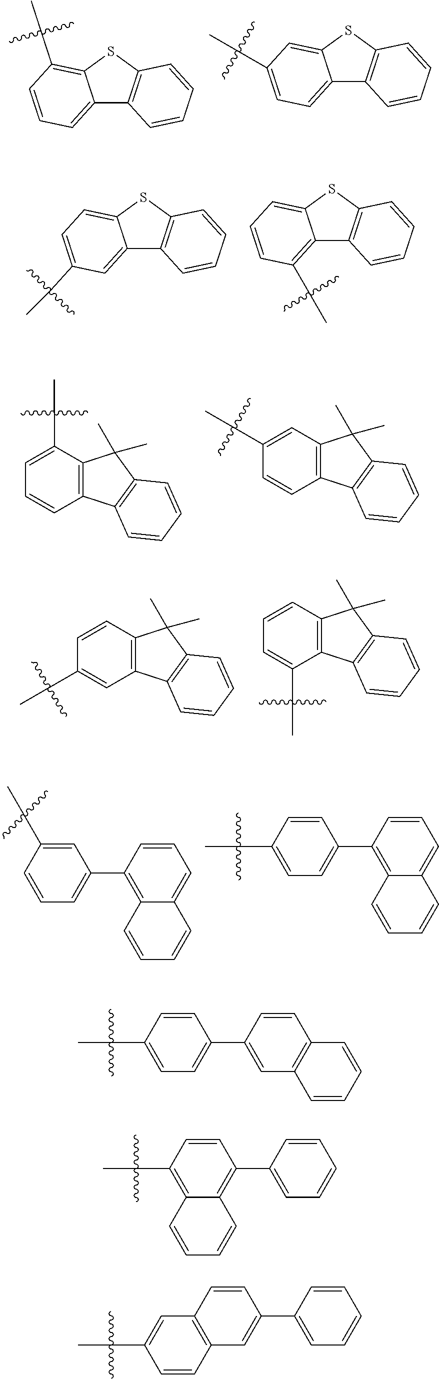

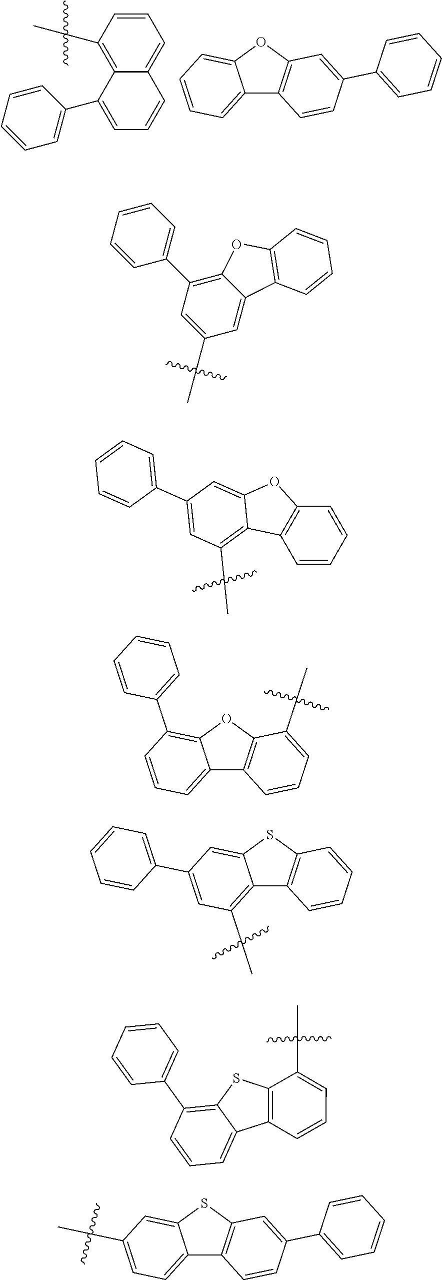

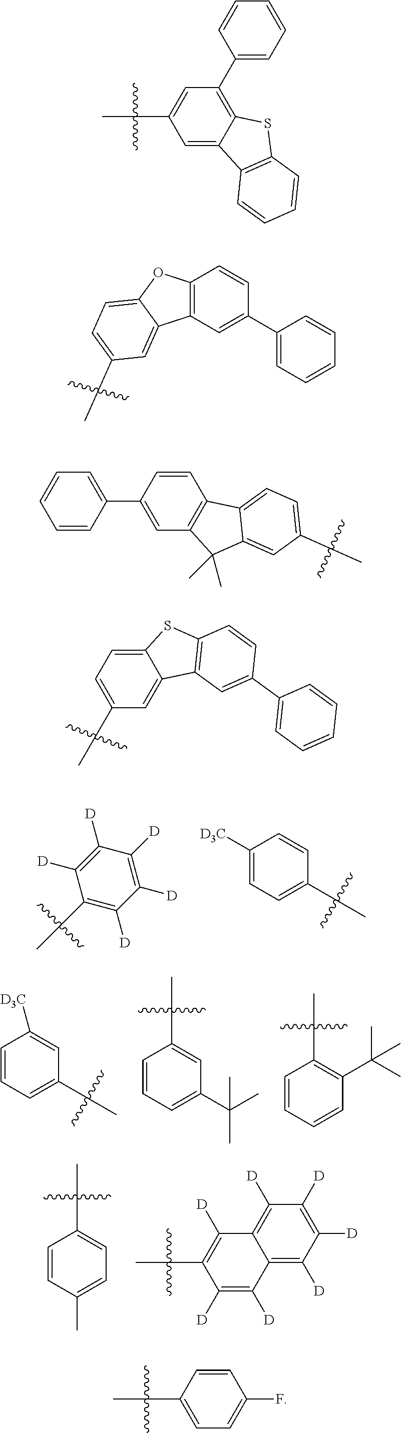

In some embodiments, L, L₁, L₂, L₃, and L₄ are identical or different, and are each independently selected from a single bond, a substituted or unsubstituted arylene having 6 to 15 carbon atoms, and a substituted or unsubstituted heteroarylene having 12 to 18 carbon atoms.

In some embodiments, L, L₁, L₂, L₃, and L₄ are identical or different, and are each independently selected from a single bond, a substituted or unsubstituted arylene having 6, 7, 8, 9, 10, 11, 12, 13, 14, or 15 carbon atoms, and a substituted or unsubstituted heteroarylene having 12, 13, 14, 15, 16, 17, or 18 carbon atoms.

Optionally, substituent(s) in L, L₁, L₂, L₃, and L₄ are each independently selected from deuterium, fluorine, cyano, an alkyl having 1 to 5 carbon atoms, a trialkylsilyl having 3 to 8 carbon atoms, a fluoroalkyl having 1 to 4 carbon atoms, a deuterated alkyl having 1 to 4 carbon atoms, phenyl, and naphthyl.

In some embodiments, L, L₁, L₂, L₃, and L₄ are identical or different, and are each independently selected from a single bond, a substituted or unsubstituted phenylene, a substituted or unsubstituted naphthylene, a substituted or unsubstituted biphenylene, a substituted or unsubstituted dibenzothienylene, a substituted or unsubstituted dibenzofuranylene, a substituted or unsubstituted fluorenylene, a substituted or unsubstituted phenanthrylene, and a substituted or unsubstituted carbazolylene.

Optionally, substituent(s) in L, L₁, L₂, L₃, and L₄ are identical or different, and are each independently selected from deuterium, fluorine, cyano, methyl, ethyl, isopropyl, tert-butyl, trifluoromethyl, trideuteromethyl, trimethylsilyl, and phenyl.

In some embodiments, L is selected from a single bond or the following groups:

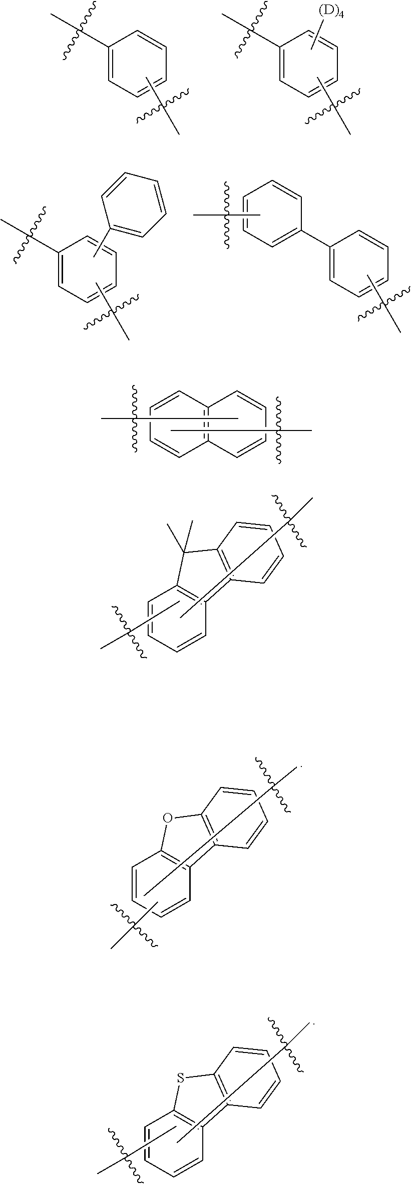

In some embodiments, L is selected from a single bond or the group consisting of the following groups:

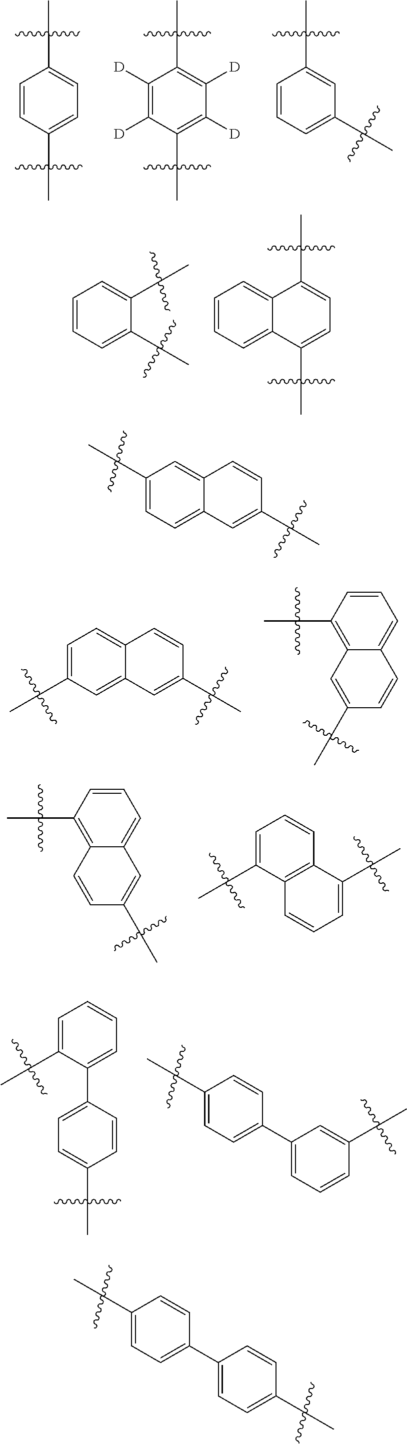

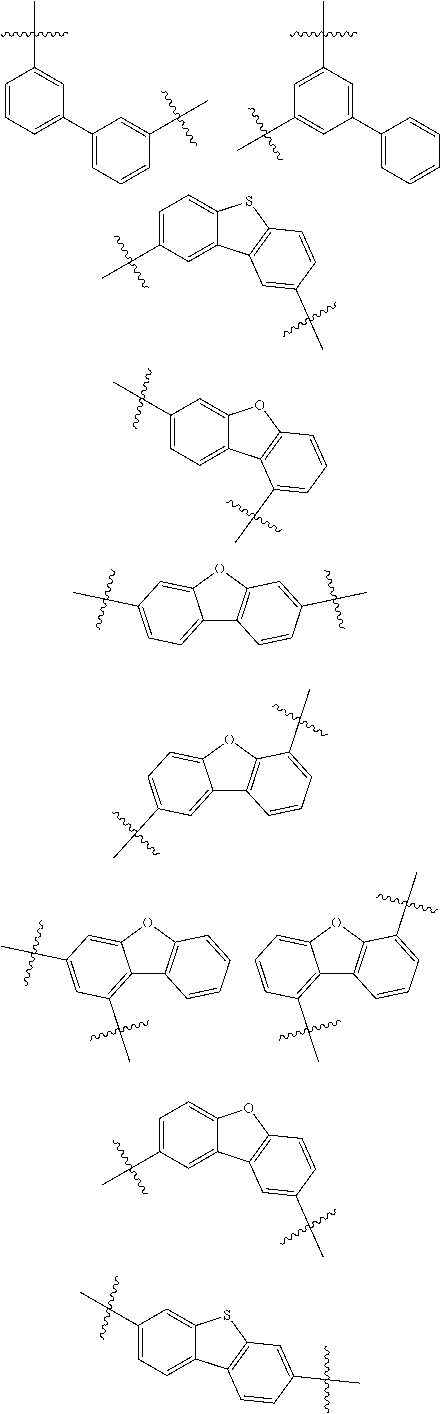

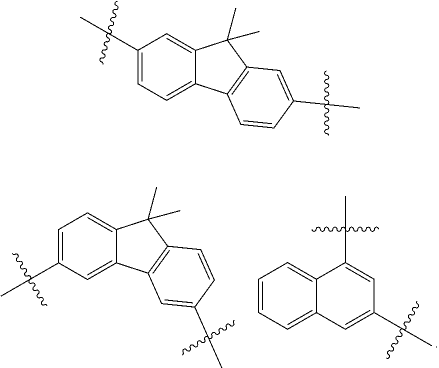

In some embodiments, L₁, L₂, L₃, and L₄ are identical or different, and are each independently selected from a single bond or the group consisting of the following groups:

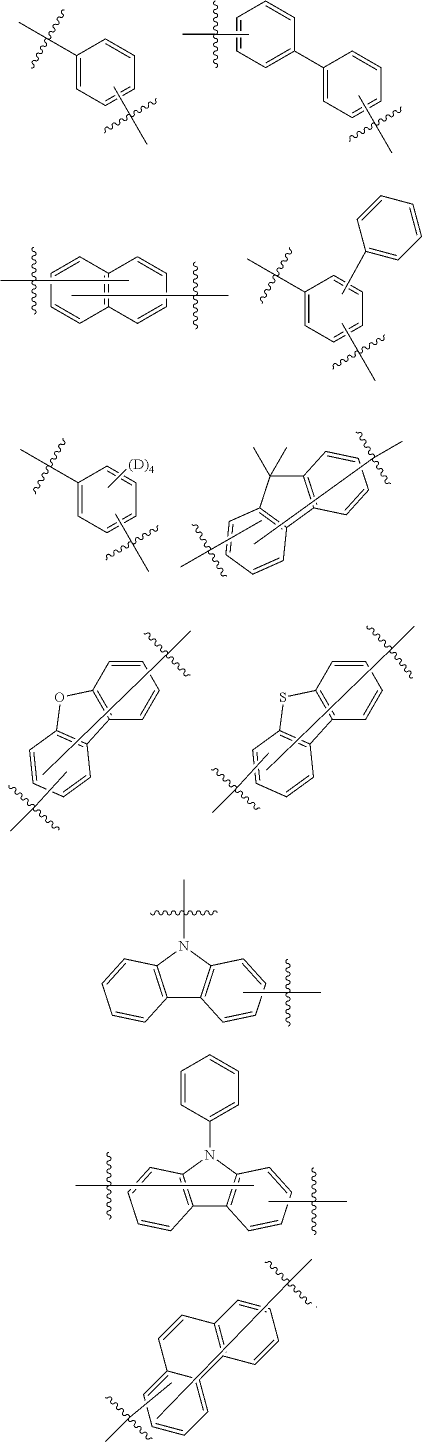

In some embodiments, L₁, L₂, L₃, and L₄ are identical or different, and are each independently selected from a single bond or the group consisting of the following groups:

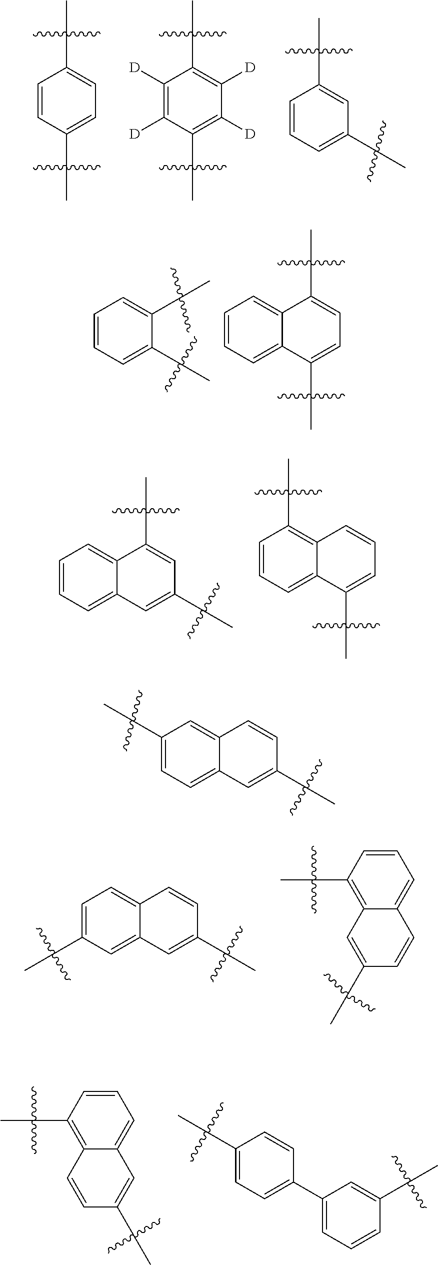

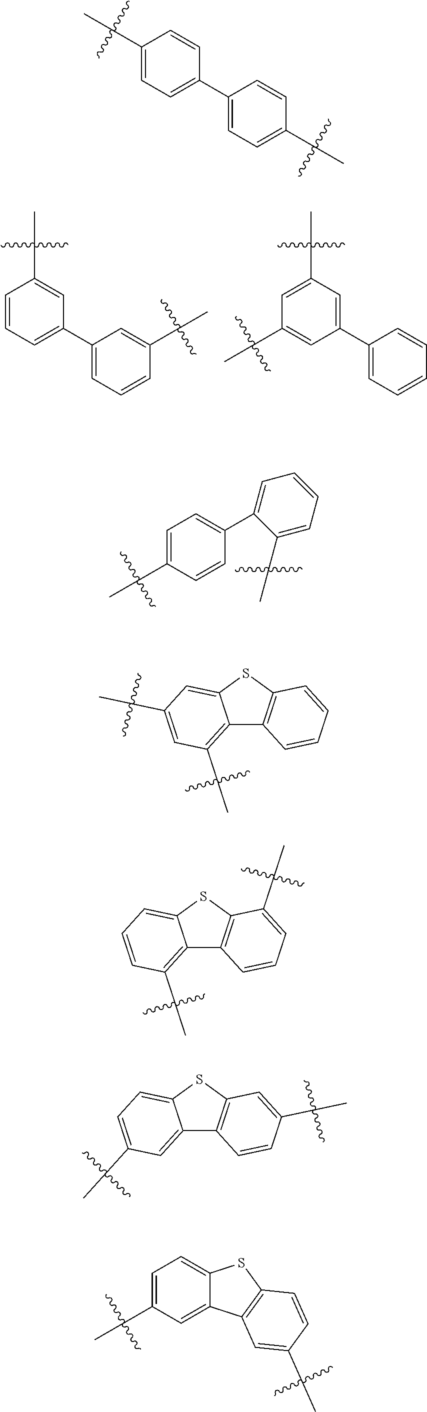

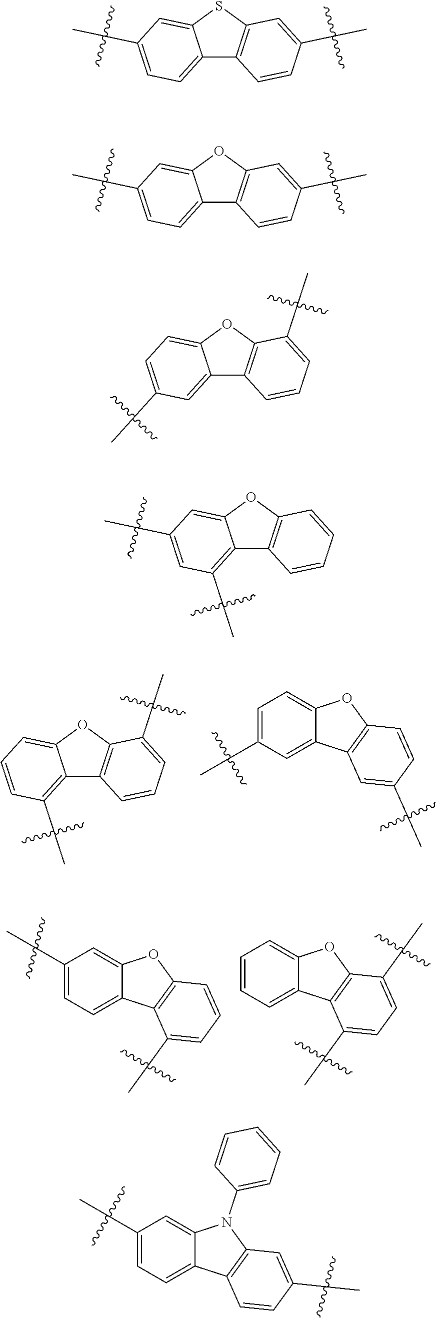

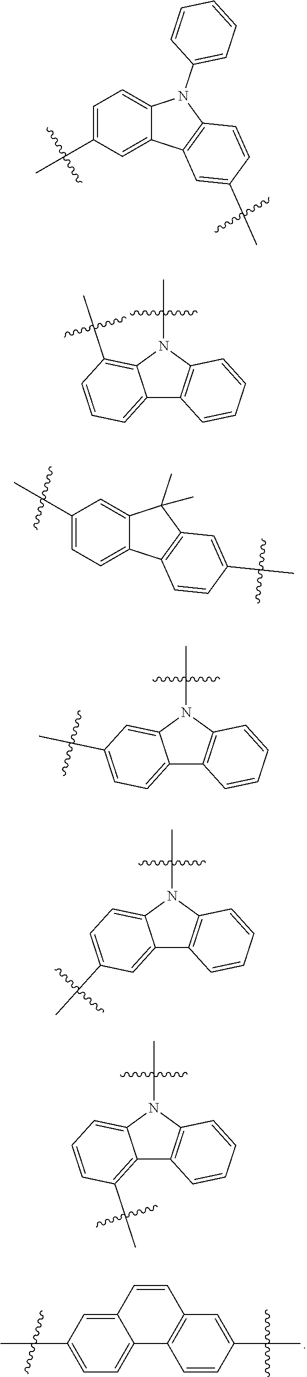

In some embodiments,

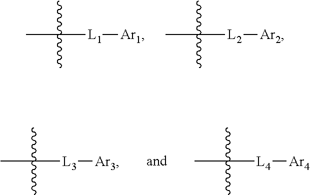

are identical or different, and are each independently selected from the following groups:

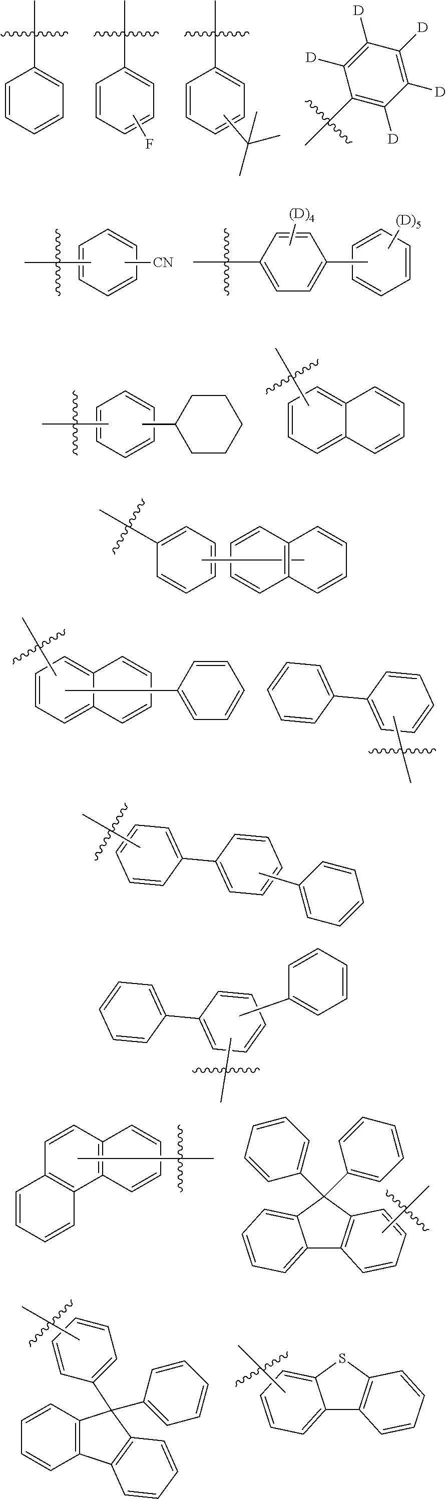

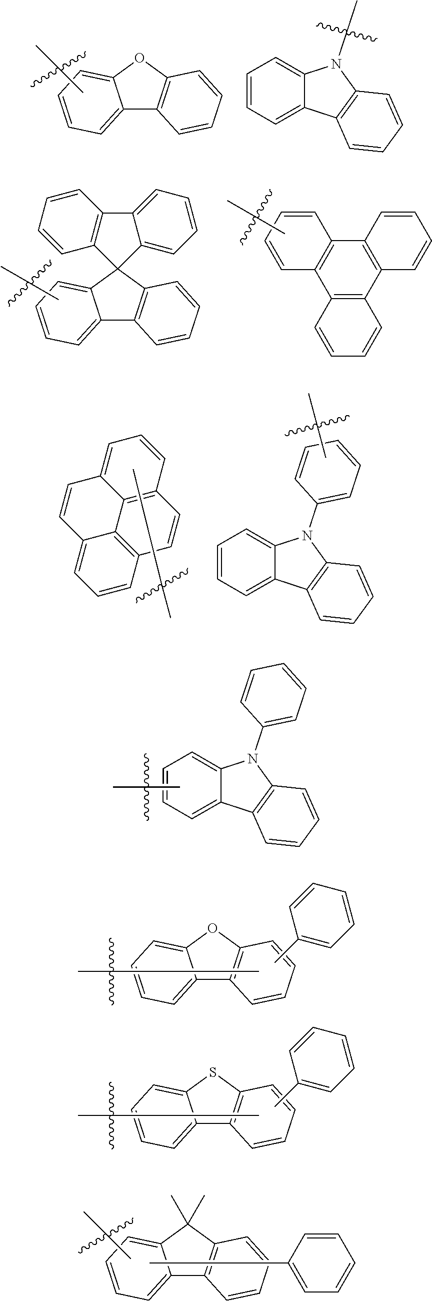

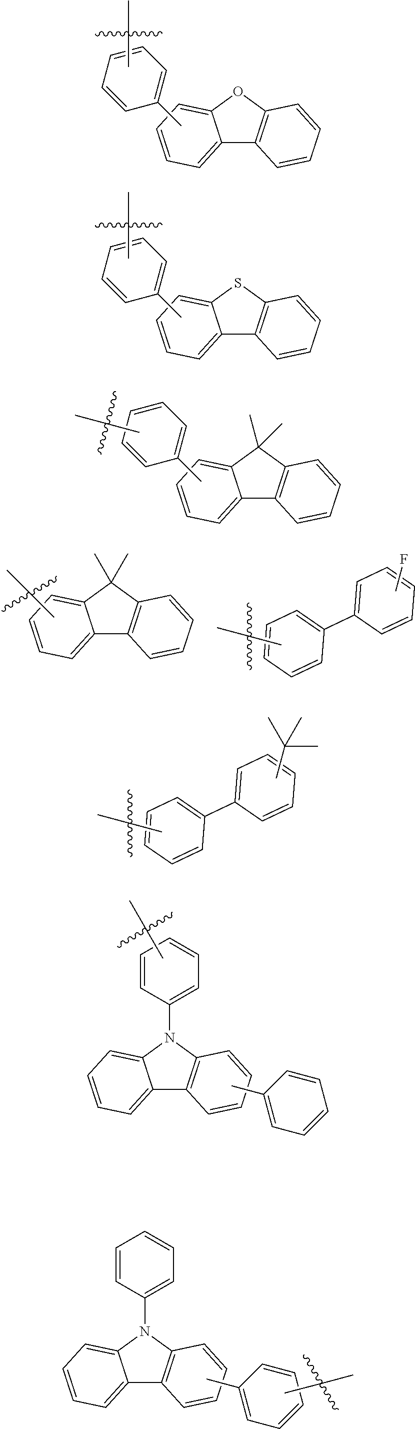

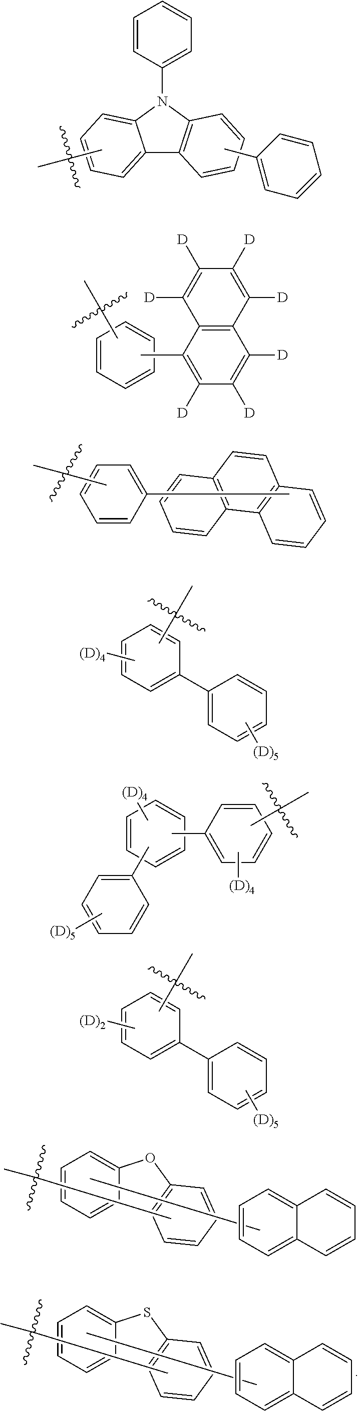

In some embodiments, group A is selected from the following groups:

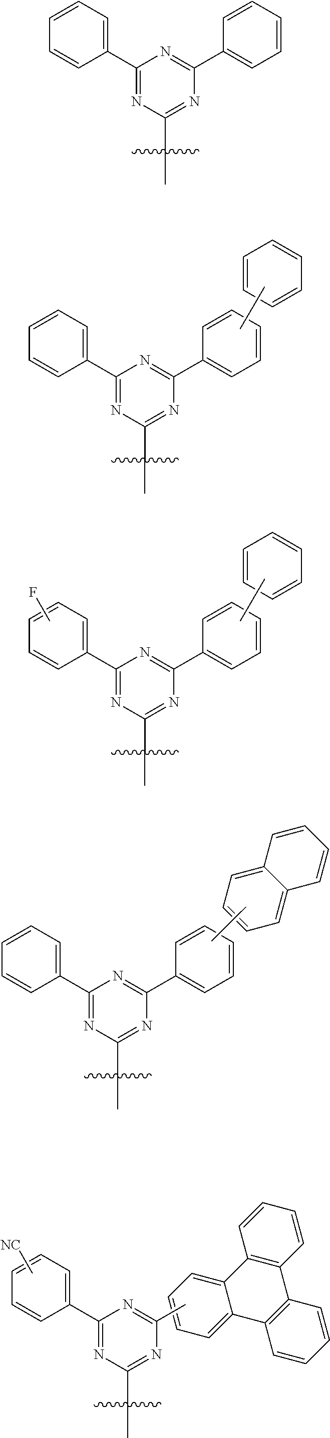

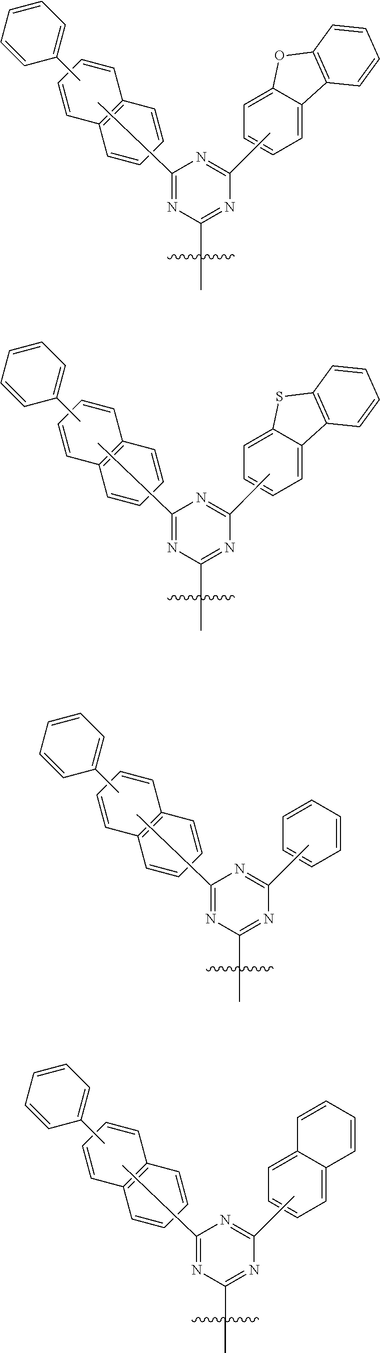

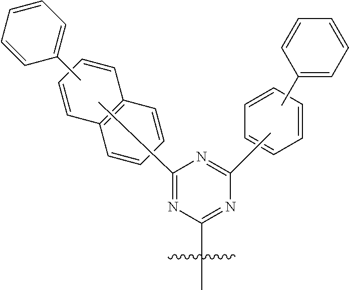

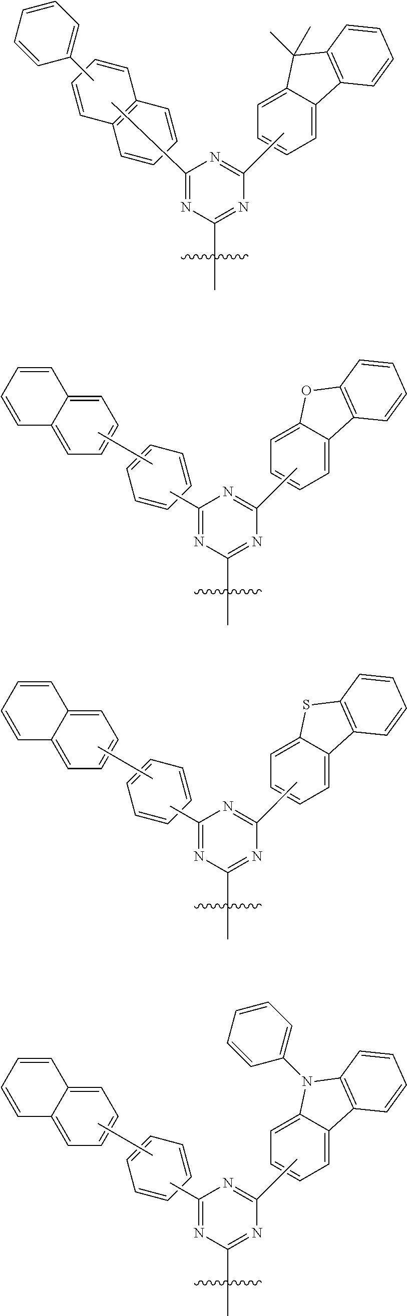

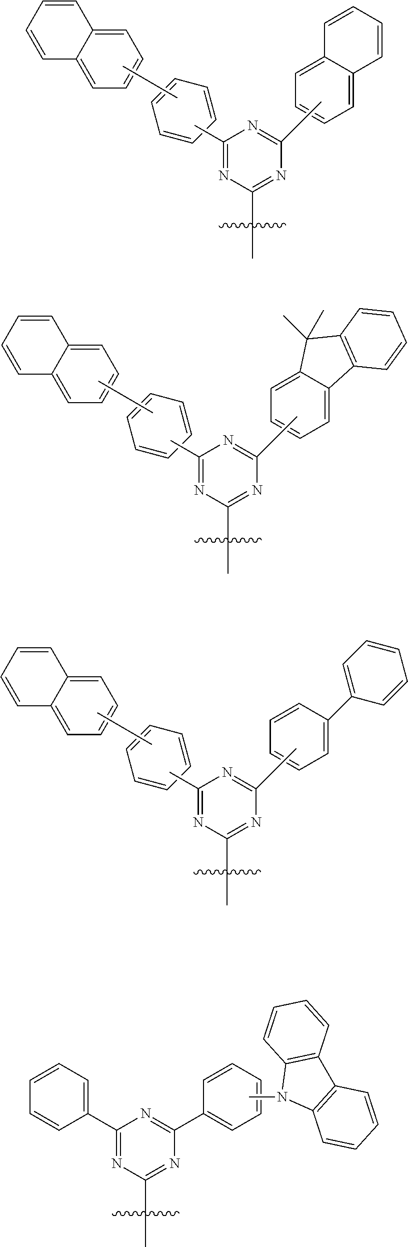

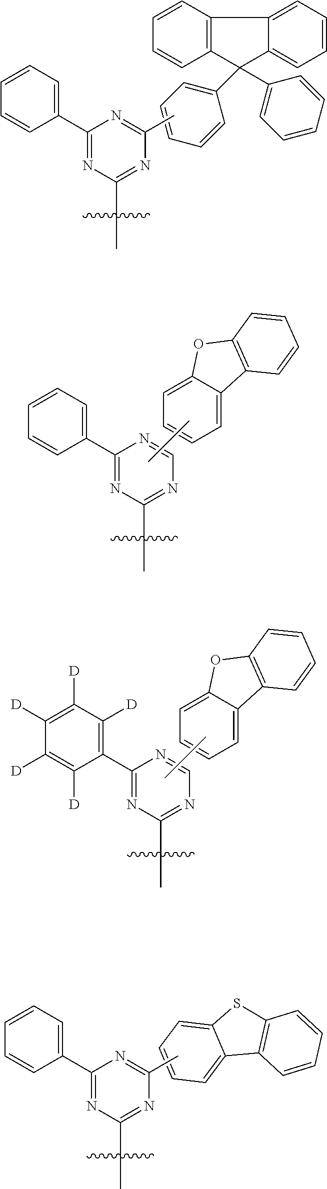

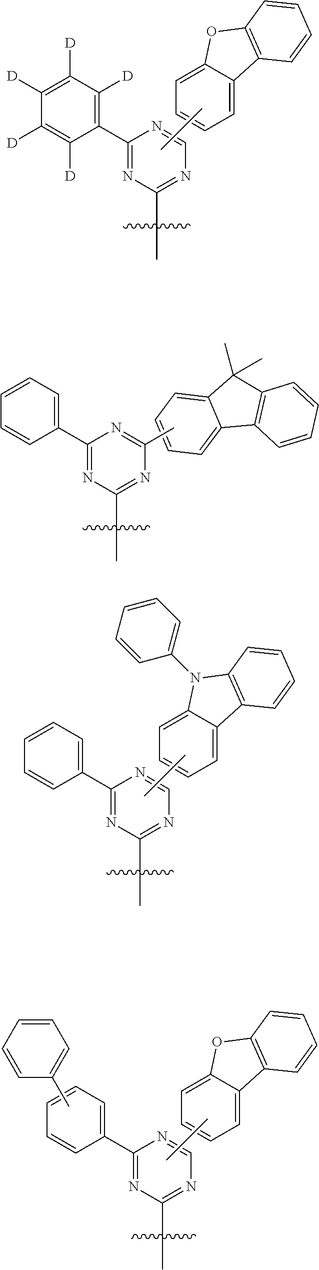

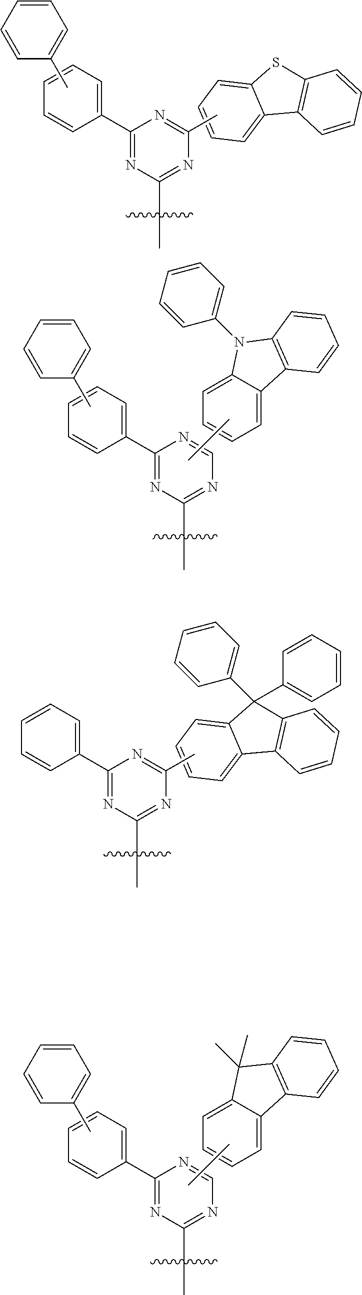

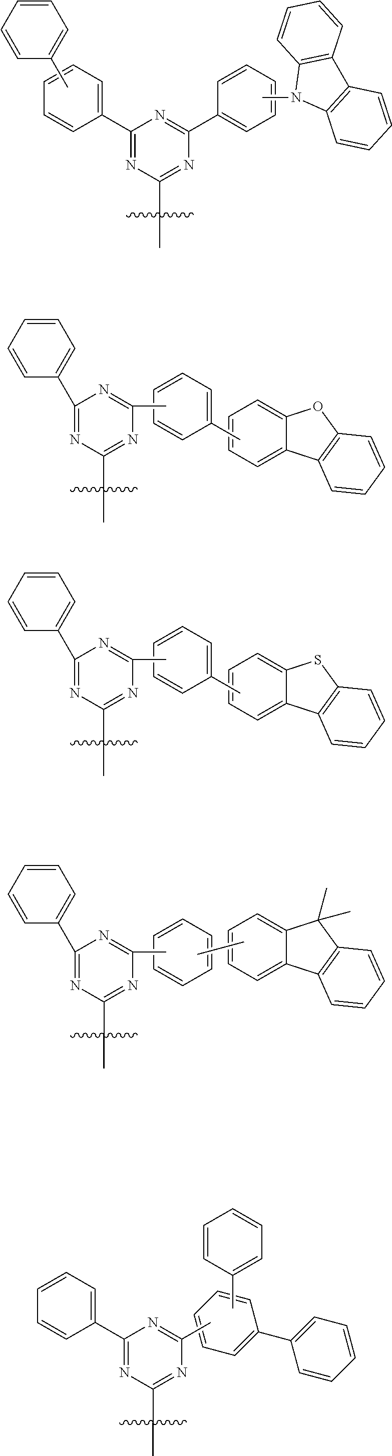

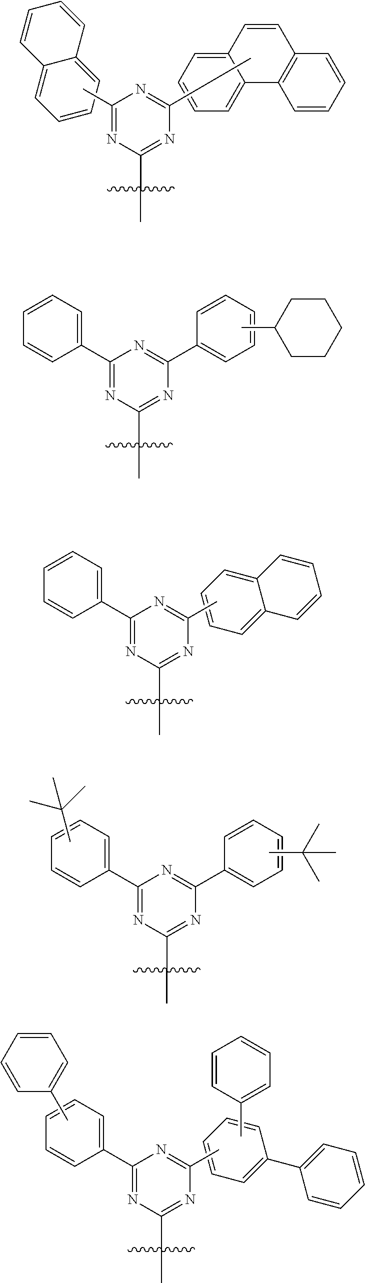

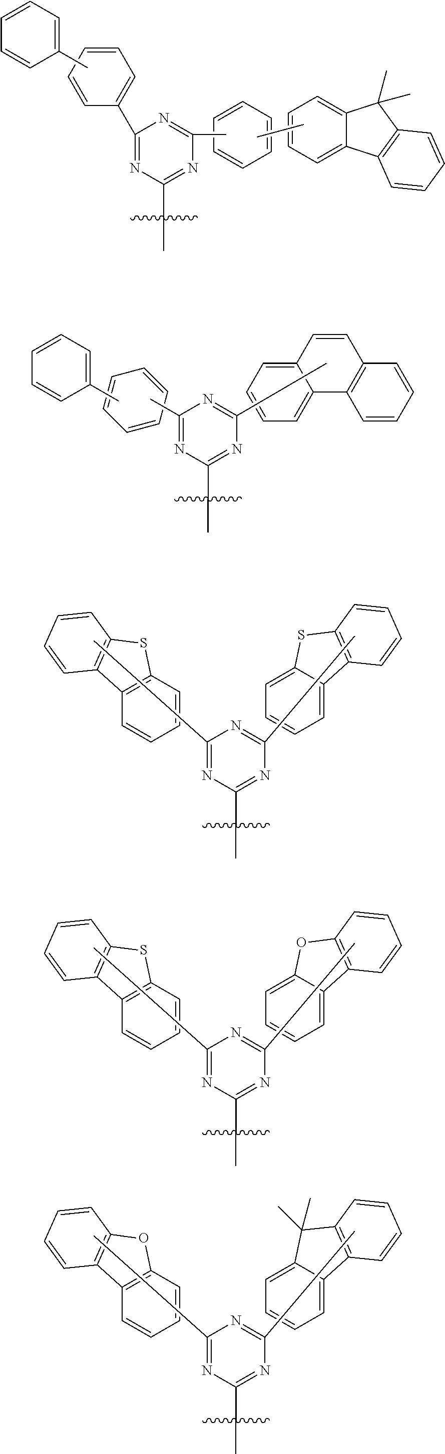

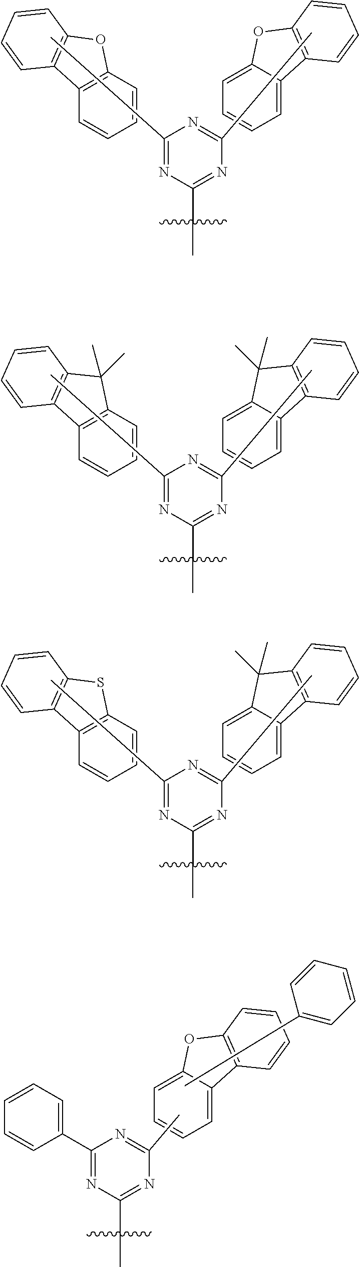

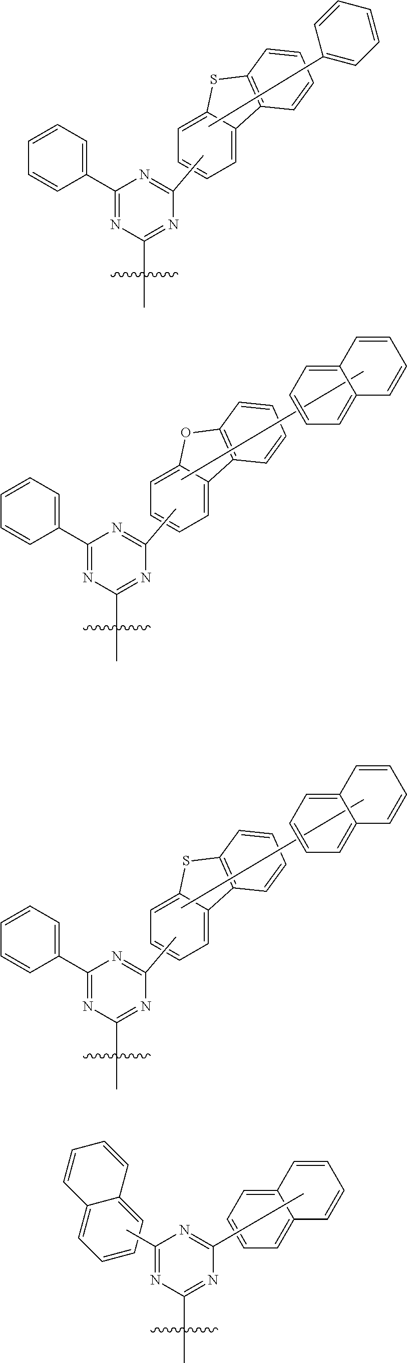

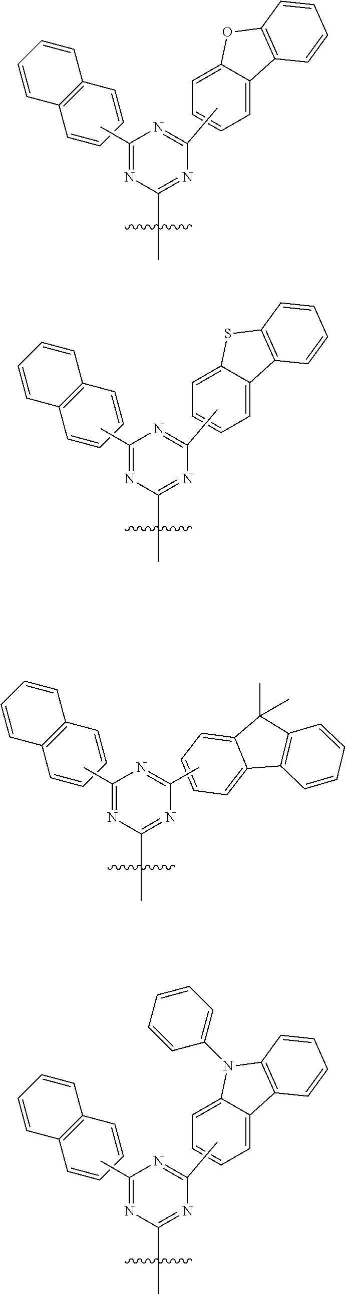

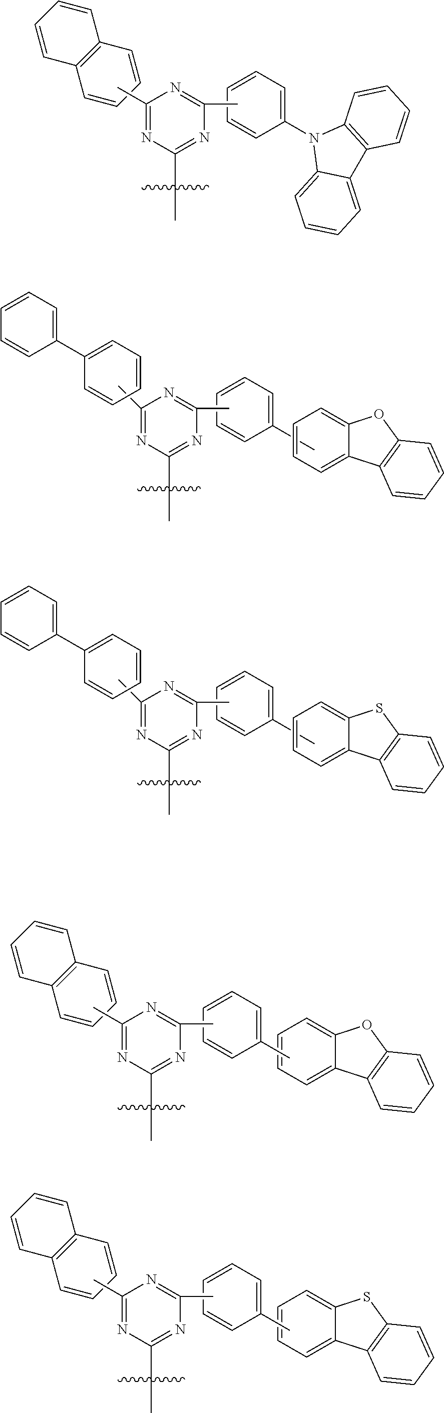

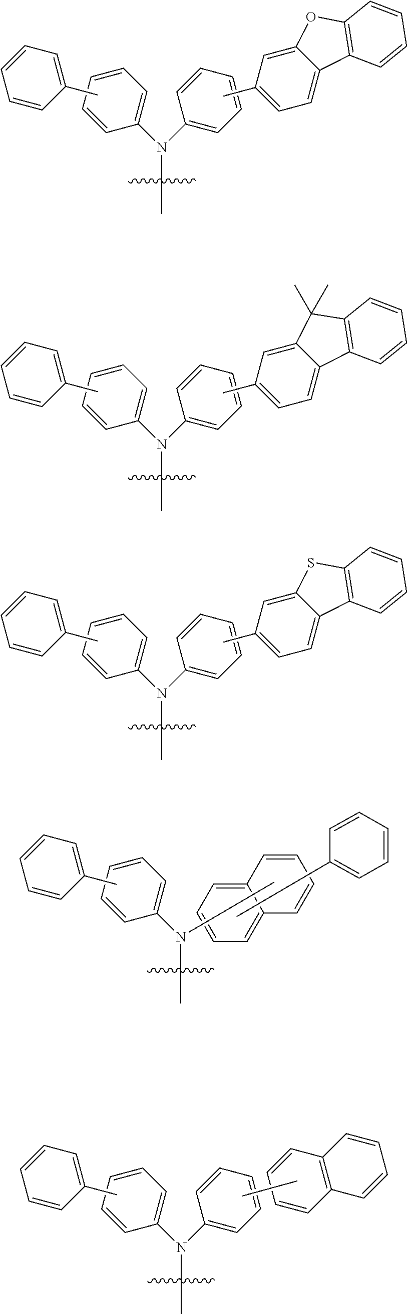

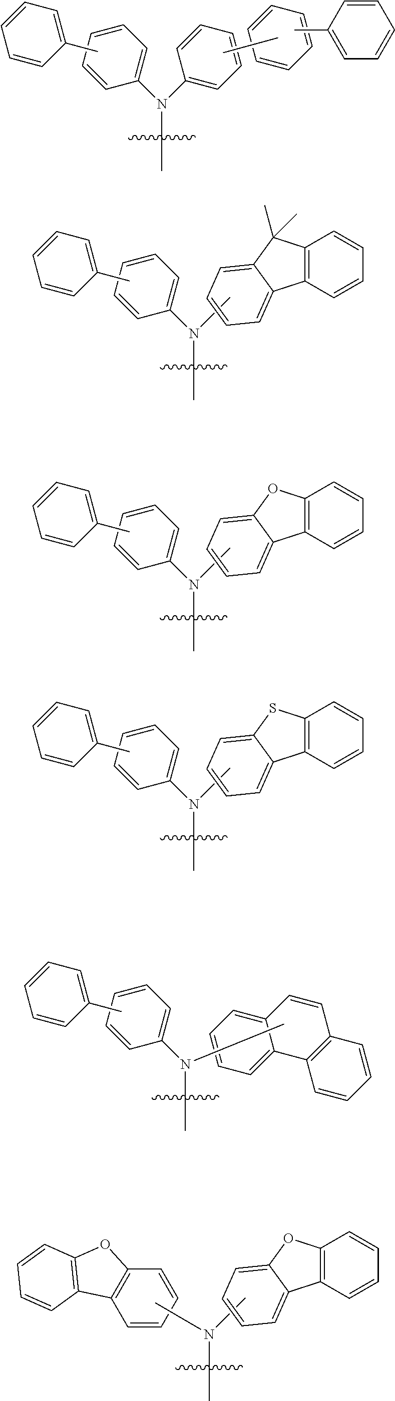

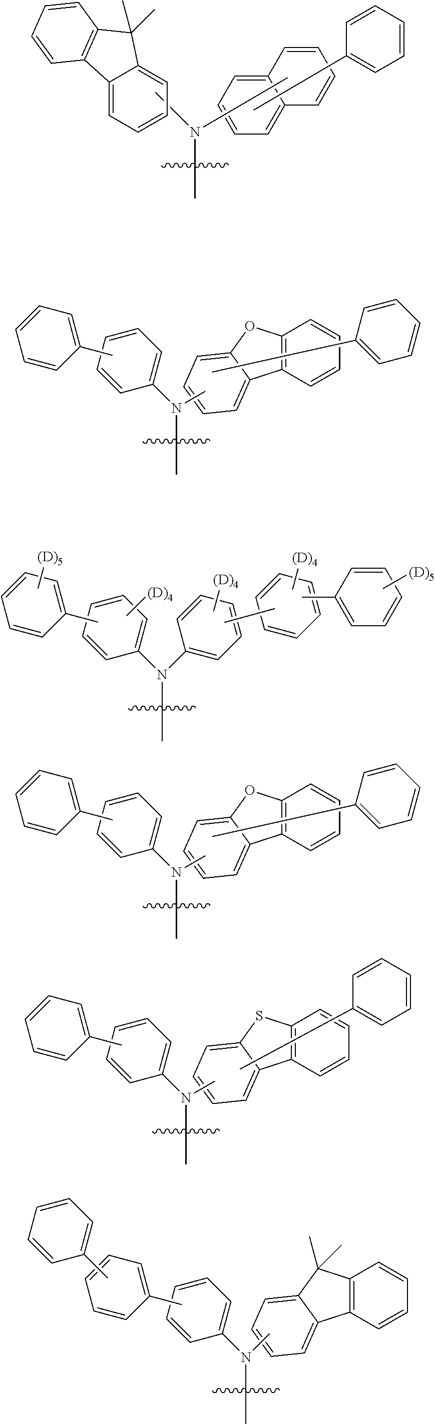

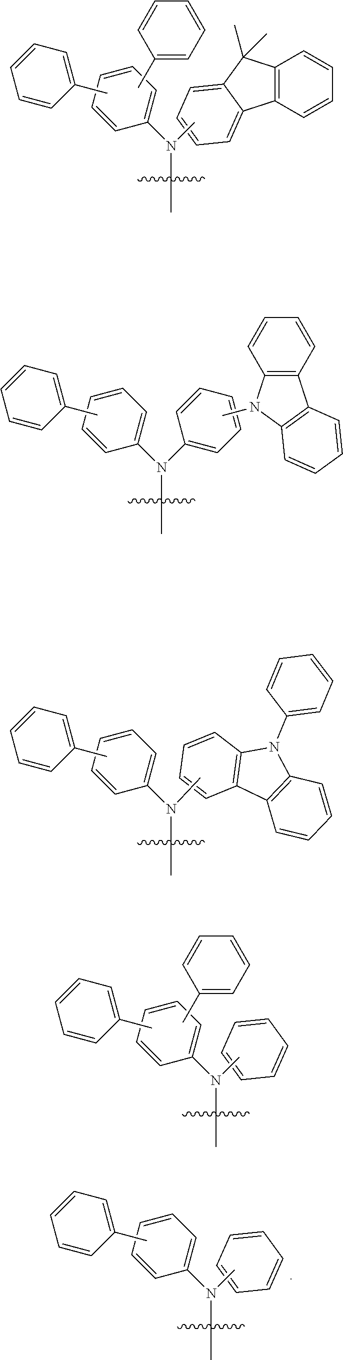

Optionally, R¹, R², and R³ are identical or different, and are each independently selected from hydrogen, deuterium, cyano, fluorine, trideuteromethyl, trialkylsilyl, trifluoromethyl, methyl, ethyl, isopropyl, tert-butyl, phenyl, naphthyl, pyridyl, dibenzofuranyl, dibenzothienyl, and carbazolyl.

Optionally, R¹, R², and R³ are identical or different, and are each independently selected from hydrogen, deuterium, and cyano.

Optionally, the nitrogen-containing compound is selected from the group consisting of the following groups:

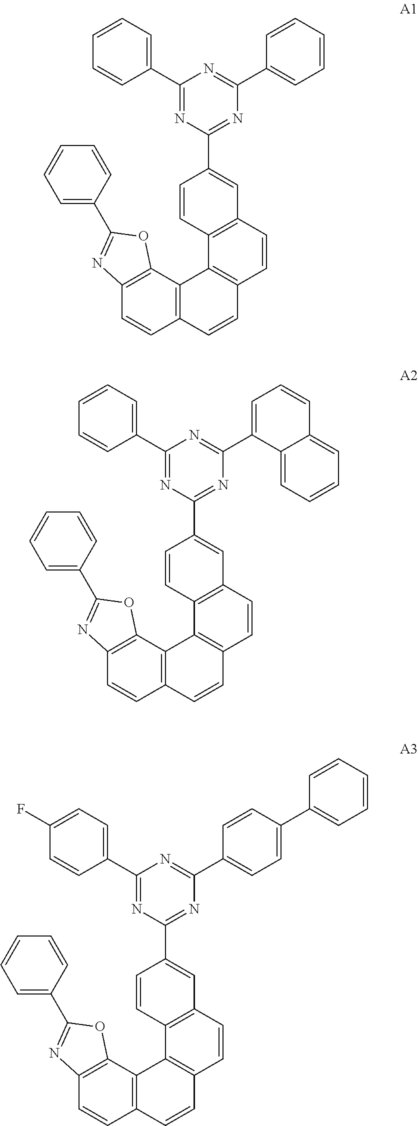 [SMILES: c1ccc(-c2nc(-c3ccccc3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3cccc4ccccc34)n2)cc1 ; Fc1ccc(-c2nc(-c3ccc(-c4ccccc4)cc3)nc(-c3ccc4c(ccc5ccc6ccc7nc(-c8ccccc8)oc7c6c54)c3)n2)cc1]

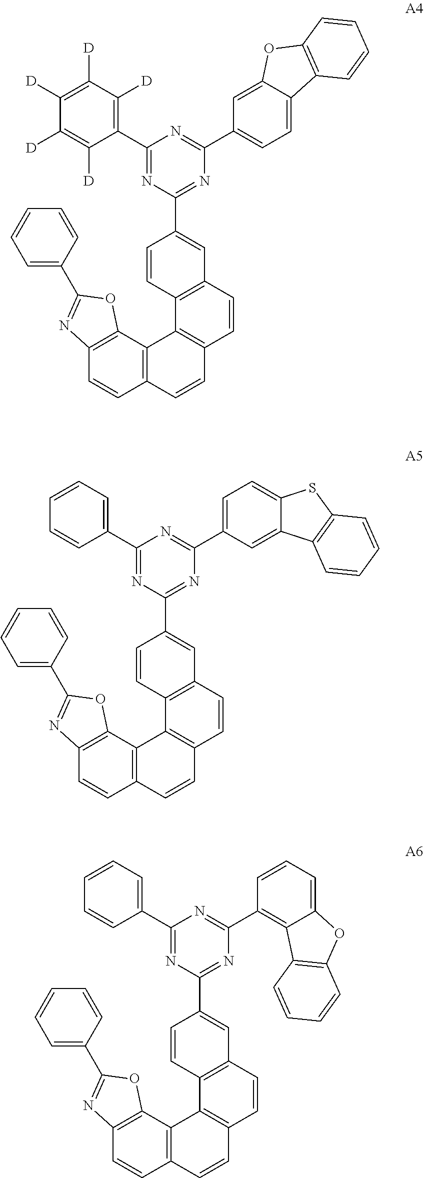 [SMILES: [2H]c1c([2H])c([2H])c(-c2nc(-c3ccc4c(ccc5ccc6ccc7nc(-c8ccccc8)oc7c6c54)c3)nc(-c3ccc4c(c3)oc3ccccc34)n2)c([2H])c1[2H] ; c1ccc(-c2nc(-c3ccc4c(ccc5ccc6ccc7nc(-c8ccccc8)oc7c6c54)c3)nc(-c3ccc4sc5ccccc5c4c3)n2)cc1 ; c1ccc(-c2nc(-c3ccc4c(ccc5ccc6ccc7nc(-c8ccccc8)oc7c6c54)c3)nc(-c3cccc4oc5ccccc5c34)n2)cc1]

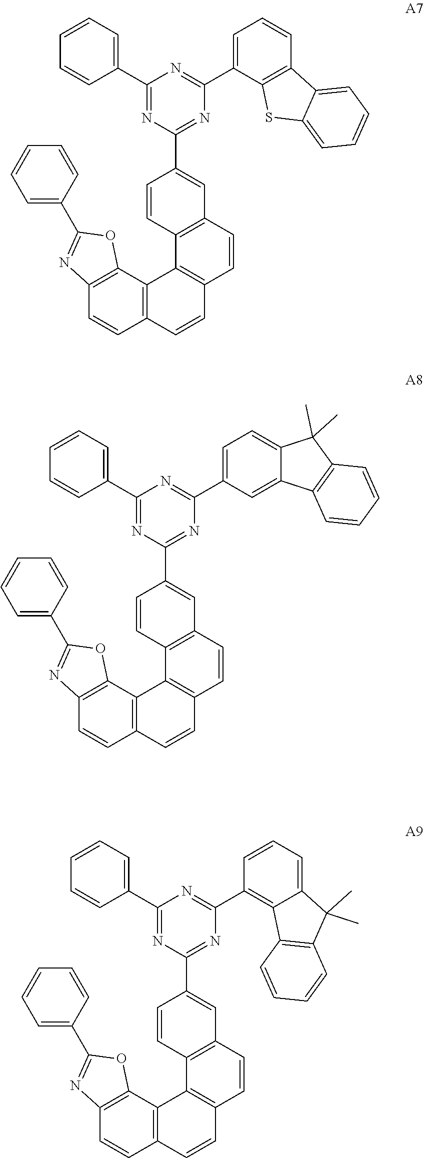 [SMILES: c1ccc(-c2nc(-c3ccc4c(ccc5ccc6ccc7nc(-c8ccccc8)oc7c6c54)c3)nc(-c3cccc4c3sc3ccccc34)n2)cc1 ; CC1(C)c2ccccc2-c2cc(-c3nc(-c4ccccc4)nc(-c4ccc5c(ccc6ccc7ccc8nc(-c9ccccc9)oc8c7c65)c4)n3)ccc21 ; CC1(C)c2ccccc2-c2c(-c3nc(-c4ccccc4)nc(-c4ccc5c(ccc6ccc7ccc8nc(-c9ccccc9)oc8c7c65)c4)n3)cccc21]

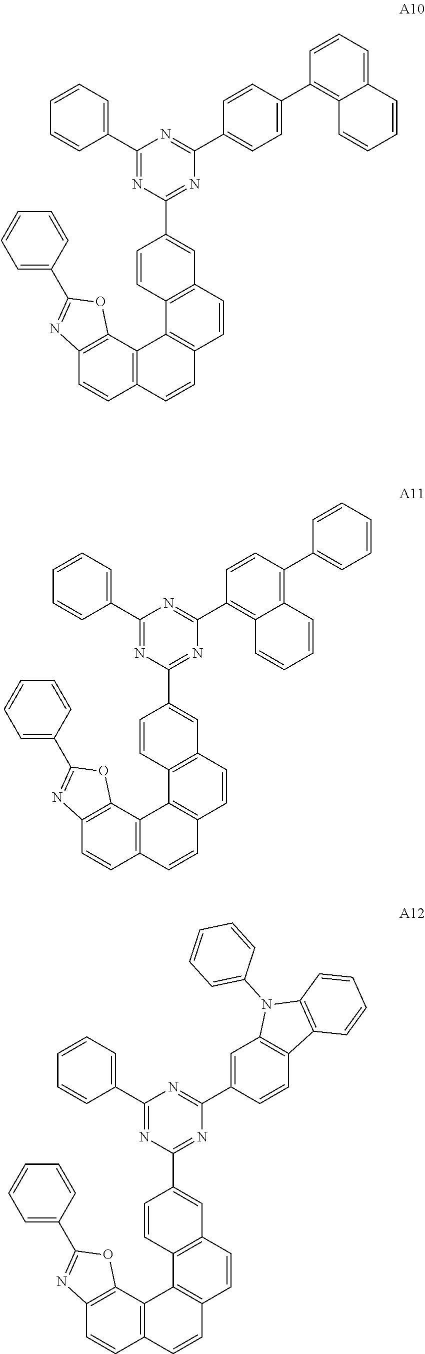 [SMILES: c1ccc(-c2nc(-c3ccc(-c4cccc5ccccc45)cc3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3ccc(-c4ccccc4)c4ccccc34)n2)cc1 ; c1ccc(-c2nc(-c3ccc4c(ccc5ccc6ccc7nc(-c8ccccc8)oc7c6c54)c3)nc(-c3ccc4c5ccccc5n(-c5ccccc5)c4c3)n2)cc1]

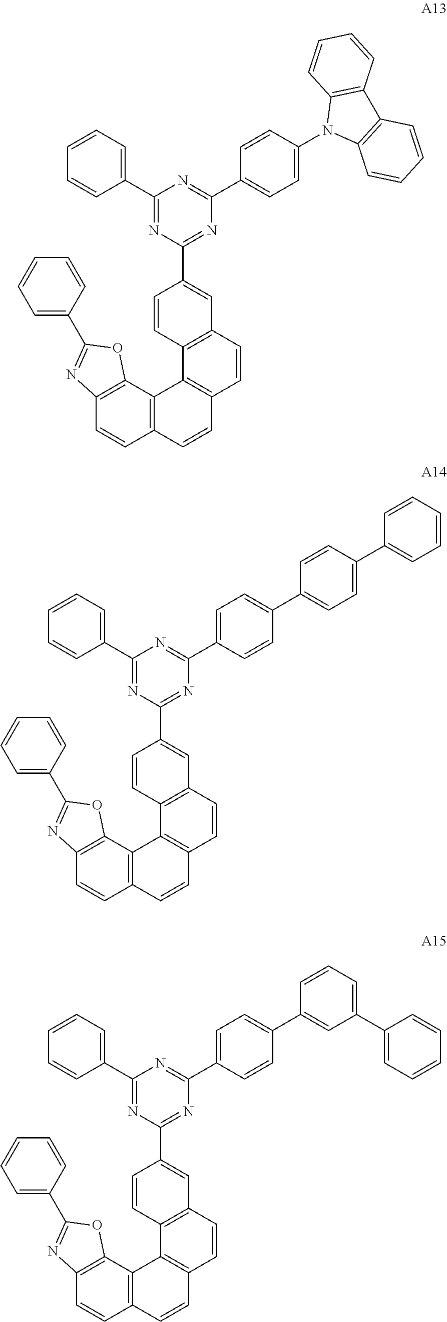 [SMILES: c1ccc(-c2nc(-c3ccc(-n4c5ccccc5c5ccccc54)cc3)nc(-c3ccc4c(ccc5ccc6ccc7nc(-c8ccccc8)oc7c6c54)c3)n2)cc1 ; c1ccc(-c2ccc(-c3ccc(-c4nc(-c5ccccc5)nc(-c5ccc6c(ccc7ccc8ccc9nc(-c%10ccccc%10)oc9c8c76)c5)n4)cc3)cc2)cc1 ; c1ccc(-c2cccc(-c3ccc(-c4nc(-c5ccccc5)nc(-c5ccc6c(ccc7ccc8ccc9nc(-c%10ccccc%10)oc9c8c76)c5)n4)cc3)c2)cc1]

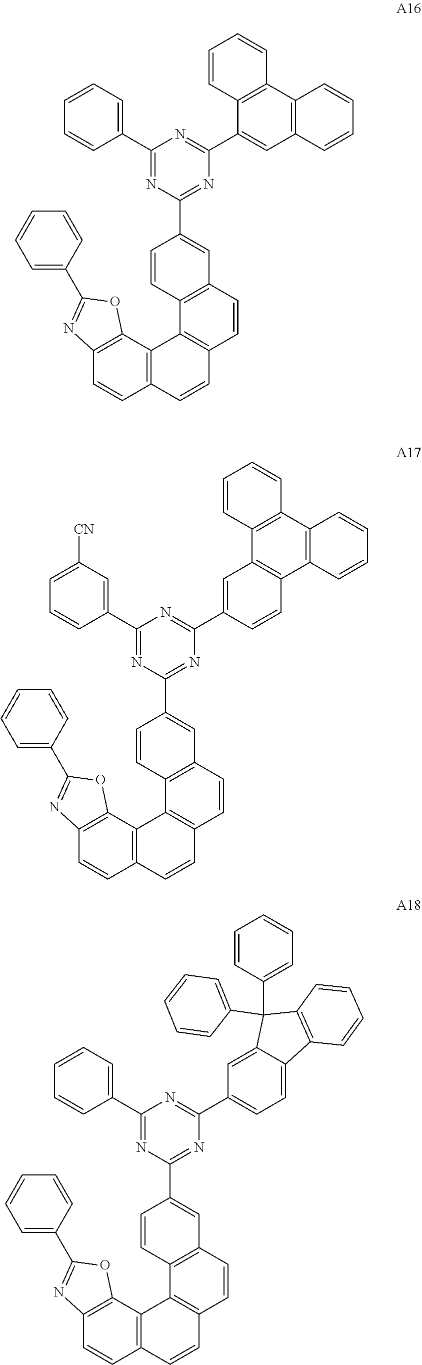 [SMILES: c1ccc(-c2nc(-c3ccc4c(ccc5ccc6ccc7nc(-c8ccccc8)oc7c6c54)c3)nc(-c3cc4ccccc4c4ccccc34)n2)cc1 ; N#Cc1cccc(-c2nc(-c3ccc4c(ccc5ccc6ccc7nc(-c8ccccc8)oc7c6c54)c3)nc(-c3ccc4c5ccccc5c5ccccc5c4c3)n2)c1 ; c1ccc(-c2nc(-c3ccc4c(c3)C(c3ccccc3)(c3ccccc3)c3ccccc3-4)nc(-c3ccc4c(ccc5ccc6ccc7nc(-c8ccccc8)oc7c6c54)c3)n2)cc1]

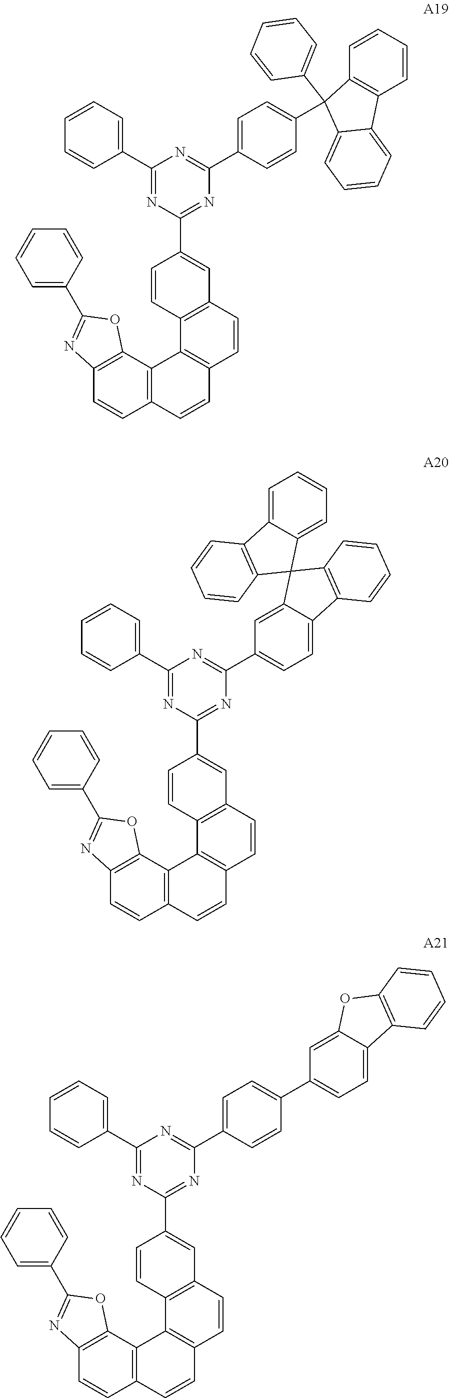 [SMILES: c1ccc(-c2nc(-c3ccc(C4(c5ccccc5)c5ccccc5-c5ccccc54)cc3)nc(-c3ccc4c(ccc5ccc6ccc7nc(-c8ccccc8)oc7c6c54)c3)n2)cc1 ; c1ccc(-c2nc(-c3ccc4c(c3)C3(c5ccccc5-c5ccccc53)c3ccccc3-4)nc(-c3ccc4c(ccc5ccc6ccc7nc(-c8ccccc8)oc7c6c54)c3)n2)cc1 ; c1ccc(-c2nc(-c3ccc(-c4ccc5c(c4)oc4ccccc45)cc3)nc(-c3ccc4c(ccc5ccc6ccc7nc(-c8ccccc8)oc7c6c54)c3)n2)cc1]

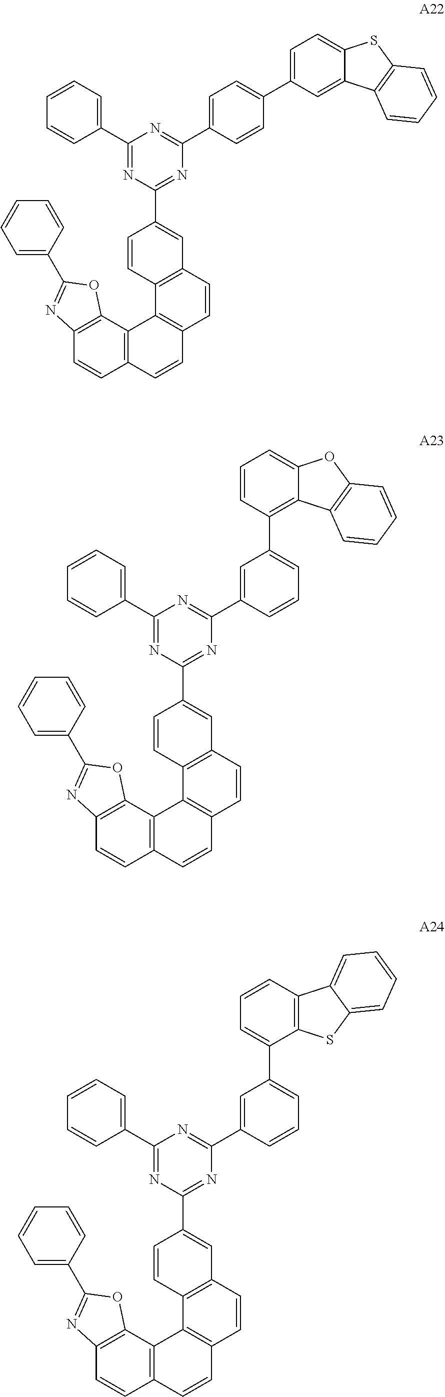 [SMILES: c1ccc(-c2nc(-c3ccc(-c4ccc5sc6ccccc6c5c4)cc3)nc(-c3ccc4c(ccc5ccc6ccc7nc(-c8ccccc8)oc7c6c54)c3)n2)cc1 ; c1ccc(-c2nc(-c3cccc(-c4cccc5oc6ccccc6c45)c3)nc(-c3ccc4c(ccc5ccc6ccc7nc(-c8ccccc8)oc7c6c54)c3)n2)cc1 ; c1ccc(-c2nc(-c3cccc(-c4cccc5c4sc4ccccc45)c3)nc(-c3ccc4c(ccc5ccc6ccc7nc(-c8ccccc8)oc7c6c54)c3)n2)cc1]

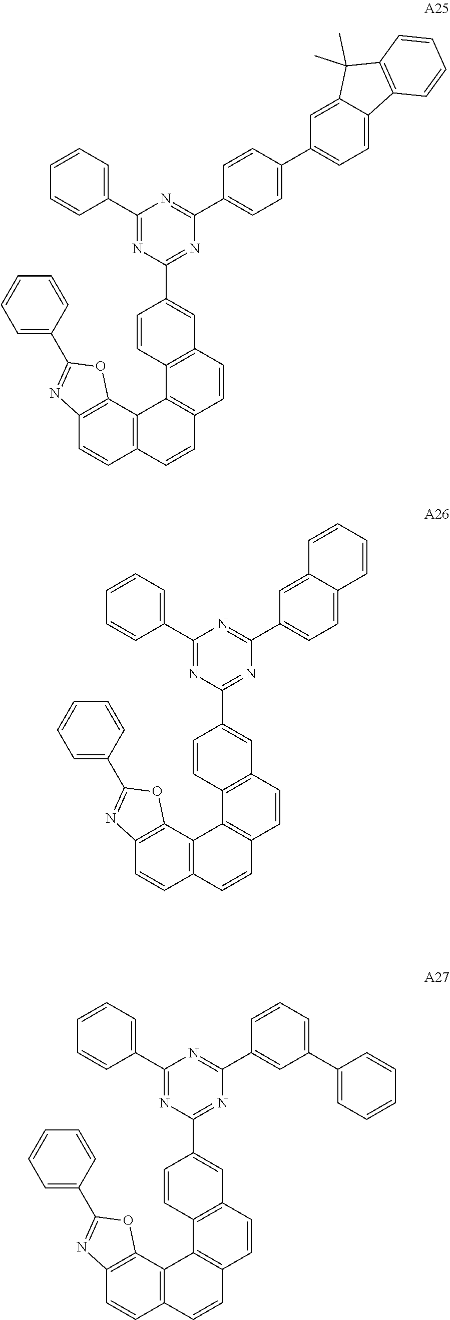 [SMILES: CC1(C)c2ccccc2-c2ccc(-c3ccc(-c4nc(-c5ccccc5)nc(-c5ccc6c(ccc7ccc8ccc9nc(-c%10ccccc%10)oc9c8c76)c5)n4)cc3)cc21 ; c1ccc(-c2nc(-c3ccc4ccccc4c3)nc(-c3ccc4c(ccc5ccc6ccc7nc(-c8ccccc8)oc7c6c54)c3)n2)cc1 ; c1ccc(-c2cccc(-c3nc(-c4ccccc4)nc(-c4ccc5c(ccc6ccc7ccc8nc(-c9ccccc9)oc8c7c65)c4)n3)c2)cc1]

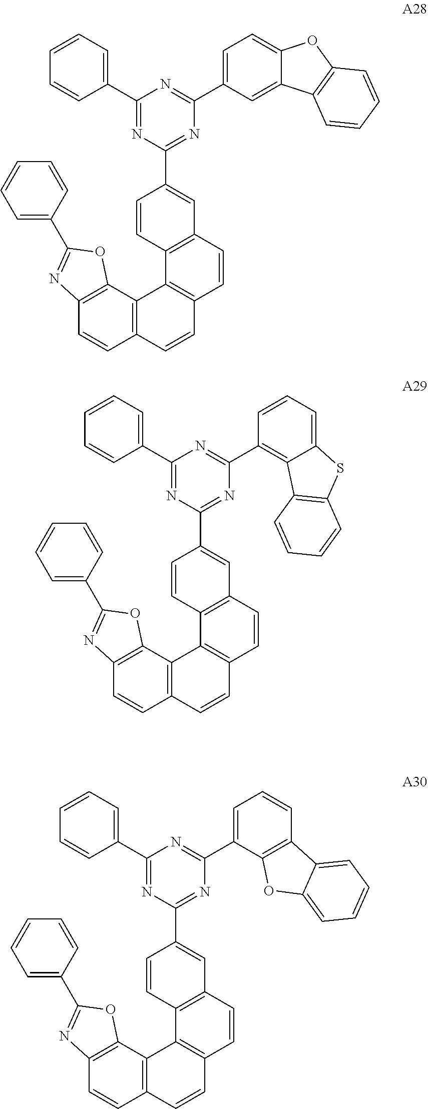

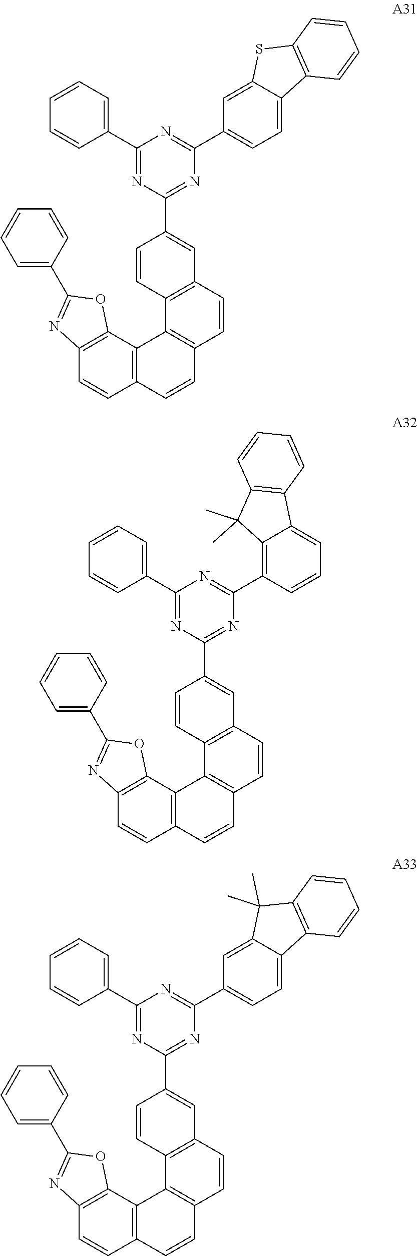 [SMILES: c1ccc(-c2nc(-c3ccc4c(ccc5ccc6ccc7nc(-c8ccccc8)oc7c6c54)c3)nc(-c3ccc4c(c3)sc3ccccc34)n2)cc1 ; CC1(C)c2ccccc2-c2cccc(-c3nc(-c4ccccc4)nc(-c4ccc5c(ccc6ccc7ccc8nc(-c9ccccc9)oc8c7c65)c4)n3)c21 ; CC1(C)c2ccccc2-c2ccc(-c3nc(-c4ccccc4)nc(-c4ccc5c(ccc6ccc7ccc8nc(-c9ccccc9)oc8c7c65)c4)n3)cc21]

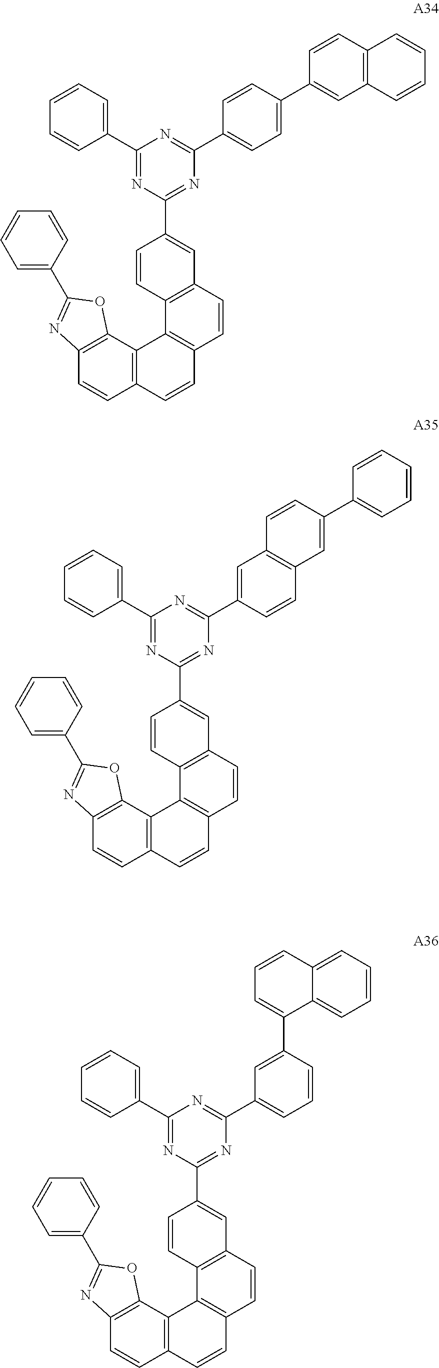 [SMILES: c1ccc(-c2nc(-c3ccc(-c4ccc5ccccc5c4)cc3)nc(-c3ccc4c(ccc5ccc6ccc7nc(-c8ccccc8)oc7c6c54)c3)n2)cc1 ; c1ccc(-c2ccc3cc(-c4nc(-c5ccccc5)nc(-c5ccc6c(ccc7ccc8ccc9nc(-c%10ccccc%10)oc9c8c76)c5)n4)ccc3c2)cc1 ; c1ccc(-c2nc(-c3cccc(-c4cccc5ccccc45)c3)nc(-c3ccc4c(ccc5ccc6ccc7nc(-c8ccccc8)oc7c6c54)c3)n2)cc1]

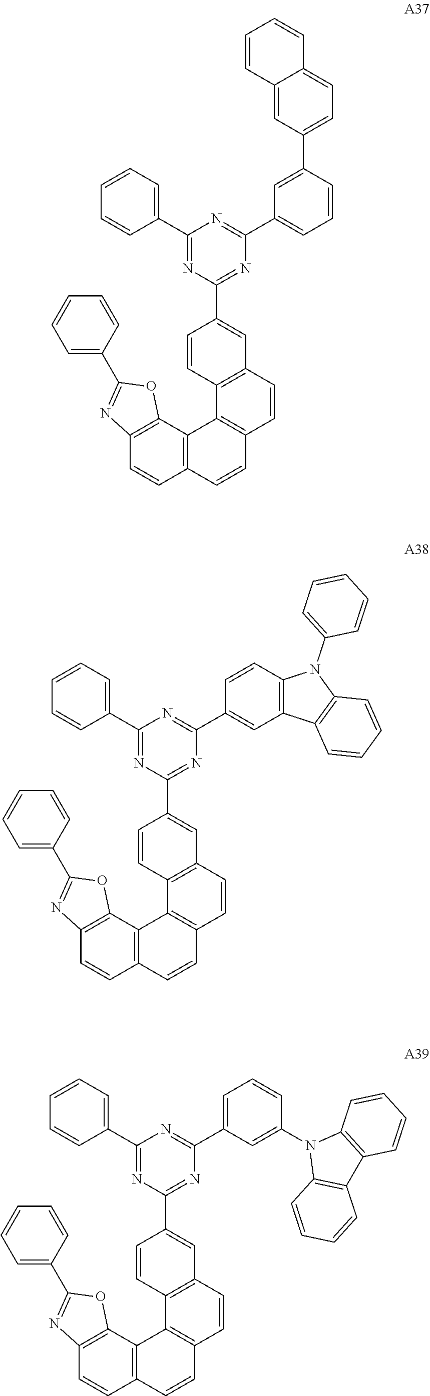 [SMILES: c1ccc(-c2nc(-c3cccc(-c4ccc5ccccc5c4)c3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3ccc4c(c3)c3ccccc3n4-c3ccccc3)n2)cc1 ; c1ccc(-c2nc(-c3cccc(-n4c5ccccc5c5ccccc54)c3)nc(-c3ccc4c(ccc5ccc6ccc7nc(-c8ccccc8)oc7c6c54)c3)n2)cc1]

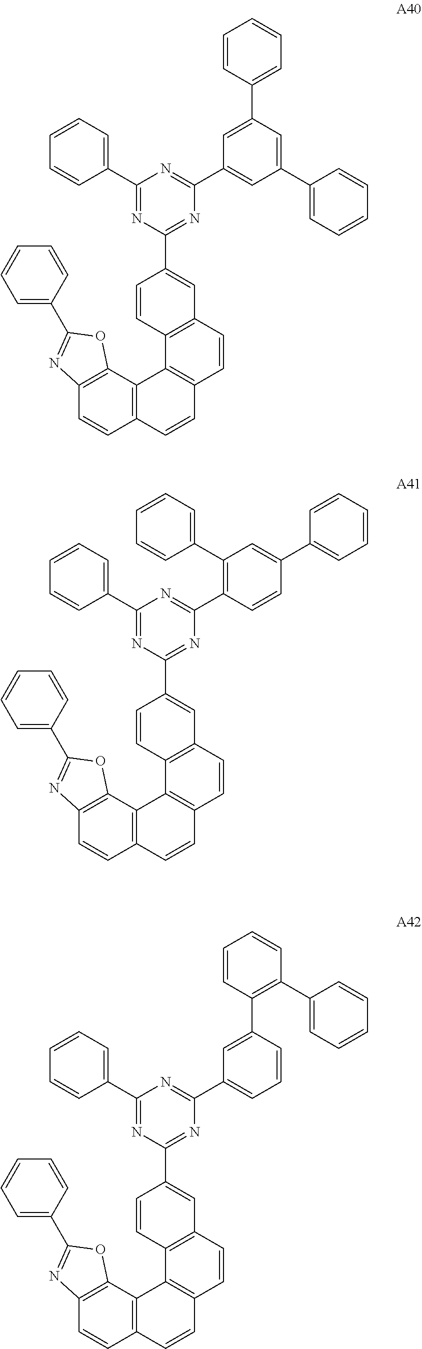 [SMILES: c1ccc(-c2cc(-c3ccccc3)cc(-c3nc(-c4ccccc4)nc(-c4ccc5c(ccc6ccc7ccc8nc(-c9ccccc9)oc8c7c65)c4)n3)c2)cc1 ; c1ccc(-c2ccc(-c3nc(-c4ccccc4)nc(-c4ccc5c(ccc6ccc7ccc8nc(-c9ccccc9)oc8c7c65)c4)n3)c(-c3ccccc3)c2)cc1 ; c1ccc(-c2nc(-c3cccc(-c4ccccc4-c4ccccc4)c3)nc(-c3ccc4c(ccc5ccc6ccc7nc(-c8ccccc8)oc7c6c54)c3)n2)cc1]

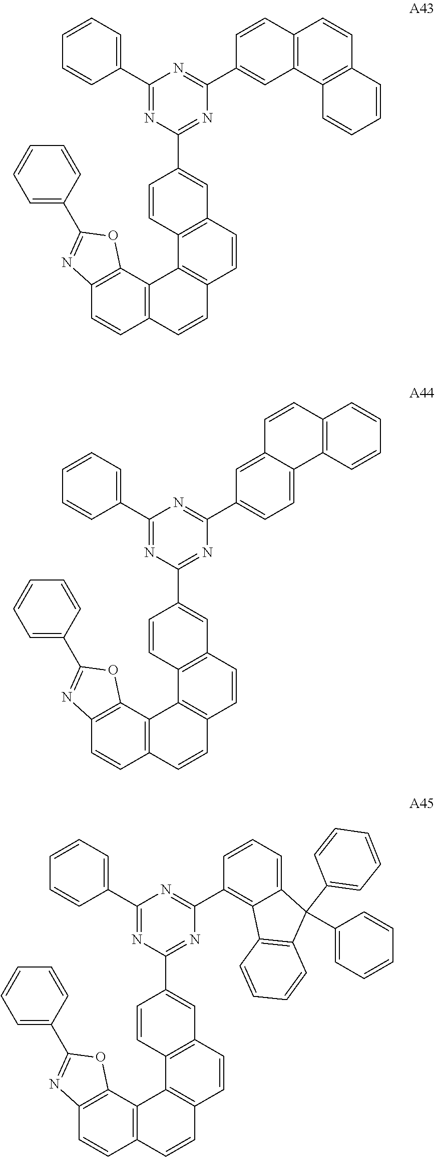 [SMILES: c1ccc(-c2nc(-c3ccc4c(ccc5ccc6ccc7nc(-c8ccccc8)oc7c6c54)c3)nc(-c3ccc4ccc5ccccc5c4c3)n2)cc1 ; c1ccc(-c2nc(-c3ccc4c(ccc5ccccc54)c3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3cccc4c3-c3ccccc3C4(c3ccccc3)c3ccccc3)n2)cc1]

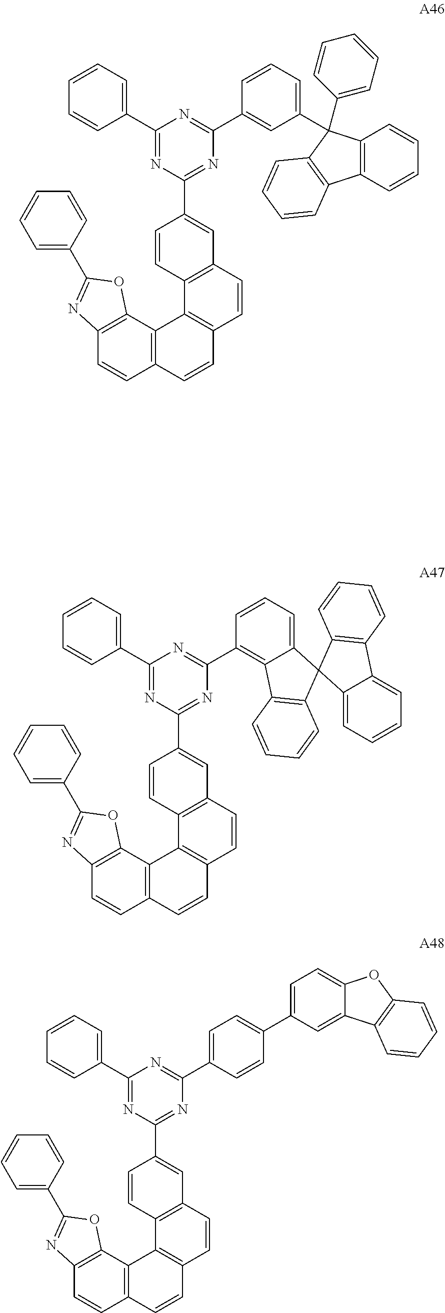 [SMILES: c1ccc(-c2nc(-c3cccc(C4(c5ccccc5)c5ccccc5-c5ccccc54)c3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3cccc4c3-c3ccccc3C43c4ccccc4-c4ccccc43)n2)cc1 ; c1ccc(-c2nc(-c3ccc(-c4ccc5oc6ccccc6c5c4)cc3)nc(-c3ccc4c(ccc5ccc6ccc7nc(-c8ccccc8)oc7c6c54)c3)n2)cc1]

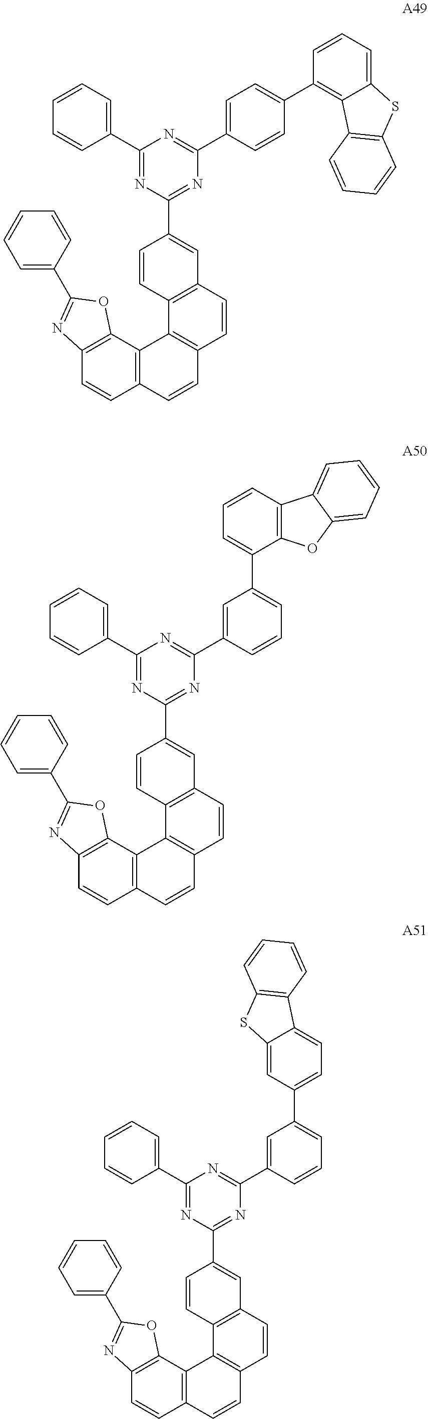 [SMILES: c1ccc(-c2nc(-c3ccc(-c4cccc5sc6ccccc6c45)cc3)nc(-c3ccc4c(ccc5ccc6ccc7nc(-c8ccccc8)oc7c6c54)c3)n2)cc1 ; c1ccc(-c2nc(-c3cccc(-c4cccc5c4oc4ccccc45)c3)nc(-c3ccc4c(ccc5ccc6ccc7nc(-c8ccccc8)oc7c6c54)c3)n2)cc1 ; c1ccc(-c2nc(-c3cccc(-c4ccc5c(c4)sc4ccccc45)c3)nc(-c3ccc4c(ccc5ccc6ccc7nc(-c8ccccc8)oc7c6c54)c3)n2)cc1]

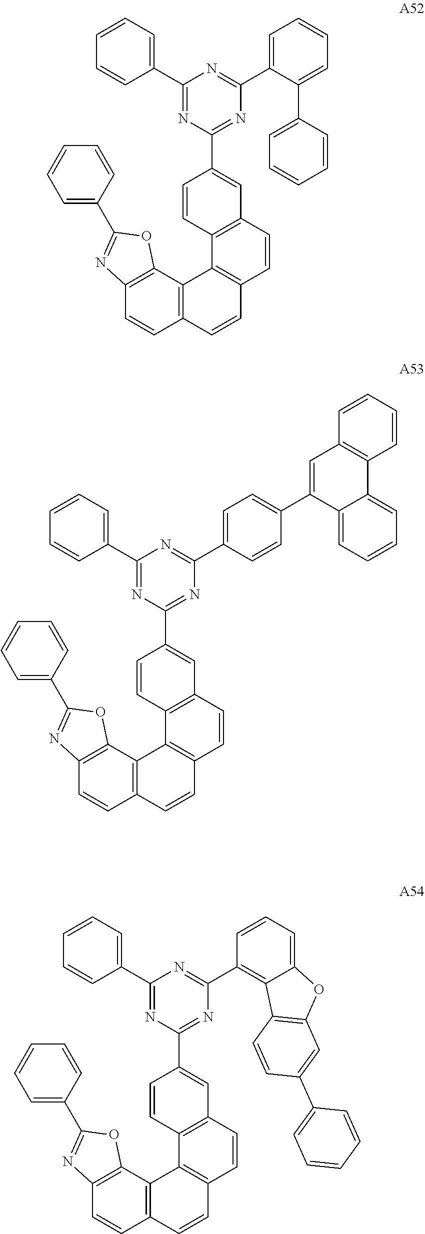 [SMILES: c1ccc(-c2nc(-c3ccc4c(ccc5ccc6ccc7nc(-c8ccccc8)oc7c6c54)c3)nc(-c3ccccc3-c3ccccc3)n2)cc1 ; c1ccc(-c2nc(-c3ccc(-c4cc5ccccc5c5ccccc45)cc3)nc(-c3ccc4c(ccc5ccc6ccc7nc(-c8ccccc8)oc7c6c54)c3)n2)cc1 ; c1ccc(-c2ccc3c(c2)oc2cccc(-c4nc(-c5ccccc5)nc(-c5ccc6c(ccc7ccc8ccc9nc(-c%10ccccc%10)oc9c8c76)c5)n4)c23)cc1]

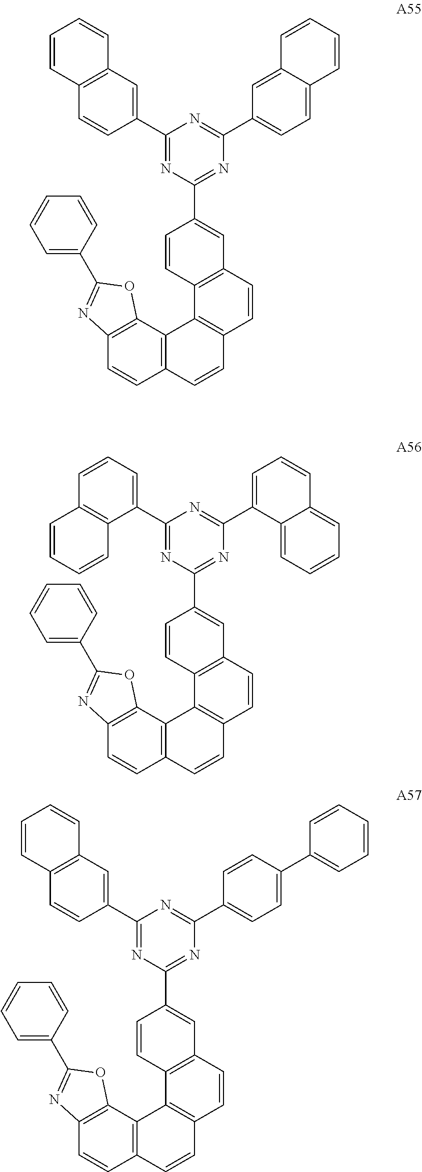 [SMILES: c1ccc(-c2nc3ccc4ccc5ccc6cc(-c7nc(-c8ccc9ccccc9c8)nc(-c8ccc9ccccc9c8)n7)ccc6c5c4c3o2)cc1 ; c1ccc(-c2nc3ccc4ccc5ccc6cc(-c7nc(-c8cccc9ccccc89)nc(-c8cccc9ccccc89)n7)ccc6c5c4c3o2)cc1 ; c1ccc(-c2ccc(-c3nc(-c4ccc5ccccc5c4)nc(-c4ccc5c(ccc6ccc7ccc8nc(-c9ccccc9)oc8c7c65)c4)n3)cc2)cc1]

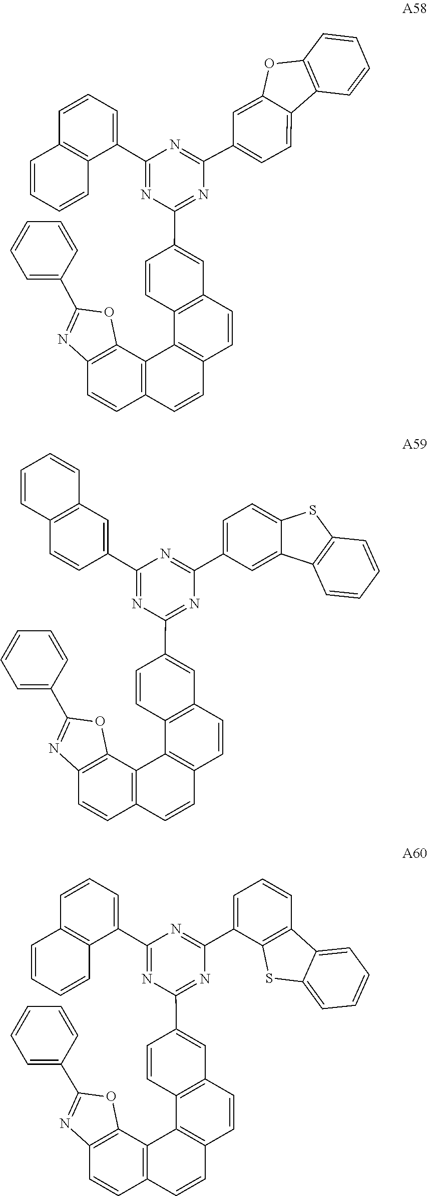 [SMILES: c1ccc(-c2nc3ccc4ccc5ccc6cc(-c7nc(-c8ccc9c(c8)oc8ccccc89)nc(-c8cccc9ccccc89)n7)ccc6c5c4c3o2)cc1 ; c1ccc(-c2nc3ccc4ccc5ccc6cc(-c7nc(-c8ccc9ccccc9c8)nc(-c8ccc9sc%10ccccc%10c9c8)n7)ccc6c5c4c3o2)cc1 ; c1ccc(-c2nc3ccc4ccc5ccc6cc(-c7nc(-c8cccc9ccccc89)nc(-c8cccc9c8sc8ccccc89)n7)ccc6c5c4c3o2)cc1]

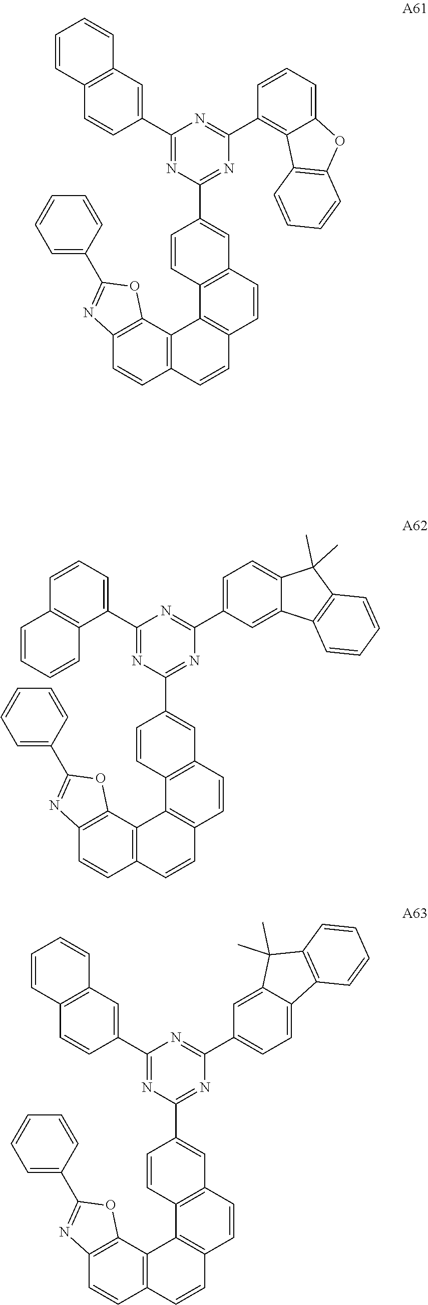 [SMILES: c1ccc(-c2nc3ccc4ccc5ccc6cc(-c7nc(-c8ccc9ccccc9c8)nc(-c8cccc9oc%10ccccc%10c89)n7)ccc6c5c4c3o2)cc1 ; CC1(C)c2ccccc2-c2cc(-c3nc(-c4ccc5c(ccc6ccc7ccc8nc(-c9ccccc9)oc8c7c65)c4)nc(-c4cccc5ccccc45)n3)ccc21 ; CC1(C)c2ccccc2-c2ccc(-c3nc(-c4ccc5ccccc5c4)nc(-c4ccc5c(ccc6ccc7ccc8nc(-c9ccccc9)oc8c7c65)c4)n3)cc21]

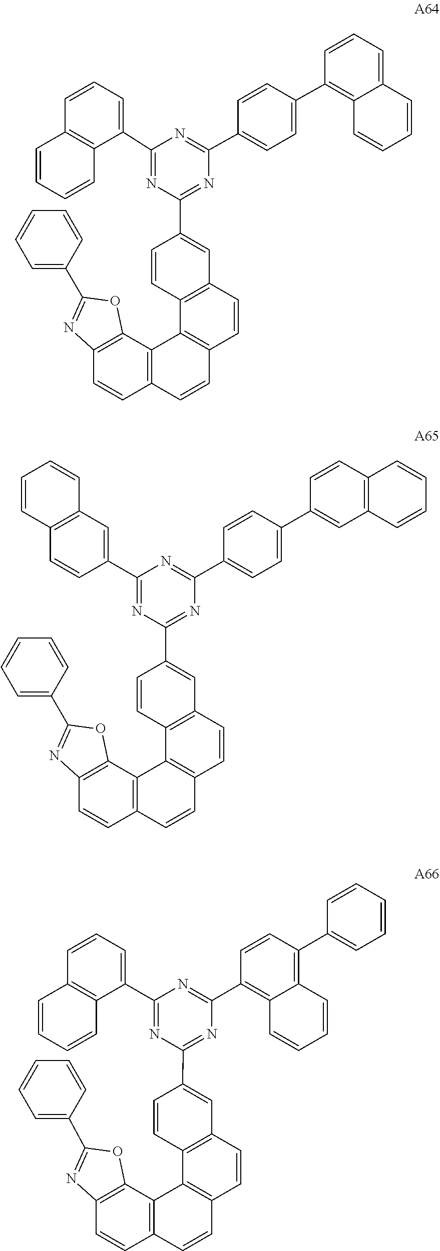 [SMILES: c1ccc(-c2nc3ccc4ccc5ccc6cc(-c7nc(-c8ccc(-c9cccc%10ccccc9%10)cc8)nc(-c8cccc9ccccc89)n7)ccc6c5c4c3o2)cc1 ; c1ccc(-c2nc3ccc4ccc5ccc6cc(-c7nc(-c8ccc(-c9ccc%10ccccc%10c9)cc8)nc(-c8ccc9ccccc9c8)n7)ccc6c5c4c3o2)cc1 ; c1ccc(-c2nc3ccc4ccc5ccc6cc(-c7nc(-c8cccc9ccccc89)nc(-c8ccc(-c9ccccc9)c9ccccc89)n7)ccc6c5c4c3o2)cc1]

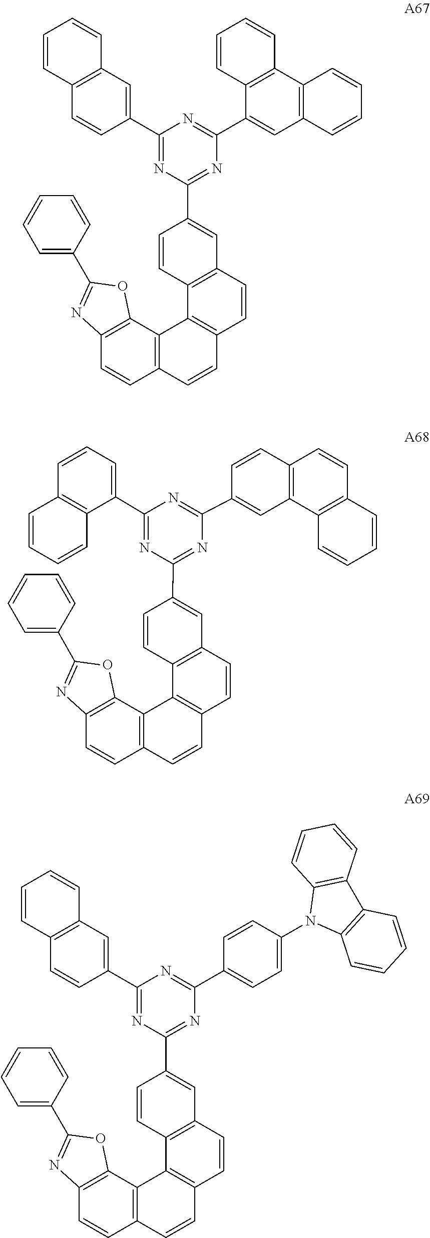 [SMILES: c1ccc(-c2nc3ccc4ccc5ccc6cc(-c7nc(-c8ccc9ccccc9c8)nc(-c8cc9ccccc9c9ccccc89)n7)ccc6c5c4c3o2)cc1 ; c1ccc(-c2nc3ccc4ccc5ccc6cc(-c7nc(-c8ccc9ccc%10ccccc%10c9c8)nc(-c8cccc9ccccc89)n7)ccc6c5c4c3o2)cc1 ; c1ccc(-c2nc3ccc4ccc5ccc6cc(-c7nc(-c8ccc(-n9c%10ccccc%10c%10ccccc%109)cc8)nc(-c8ccc9ccccc9c8)n7)ccc6c5c4c3o2)cc1]

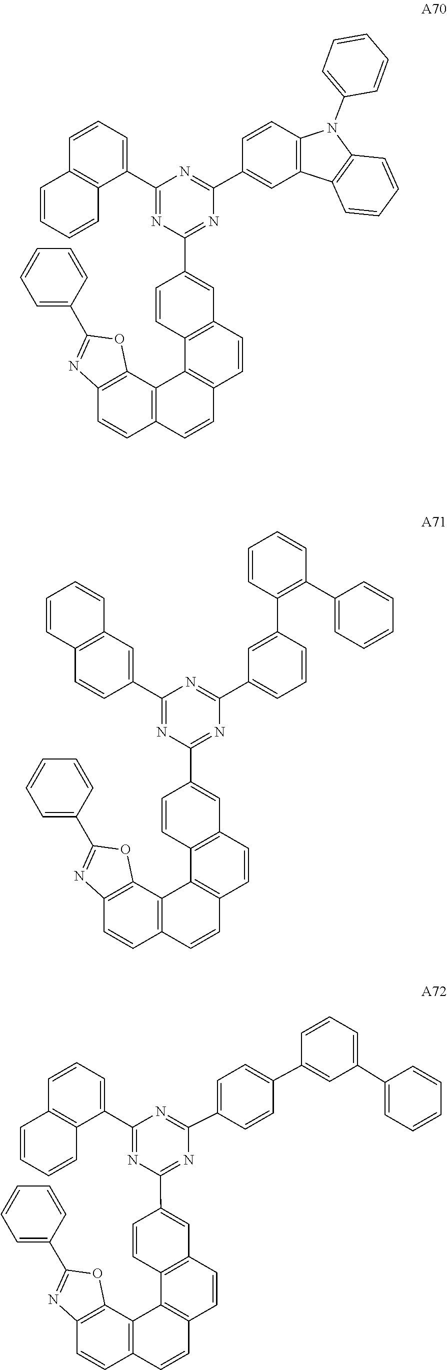 [SMILES: c1ccc(-c2nc3ccc4ccc5ccc6cc(-c7nc(-c8ccc9c(c8)c8ccccc8n9-c8ccccc8)nc(-c8cccc9ccccc89)n7)ccc6c5c4c3o2)cc1 ; c1ccc(-c2nc3ccc4ccc5ccc6cc(-c7nc(-c8cccc(-c9ccccc9-c9ccccc9)c8)nc(-c8ccc9ccccc9c8)n7)ccc6c5c4c3o2)cc1 ; c1ccc(-c2cccc(-c3ccc(-c4nc(-c5ccc6c(ccc7ccc8ccc9nc(-c%10ccccc%10)oc9c8c76)c5)nc(-c5cccc6ccccc56)n4)cc3)c2)cc1]

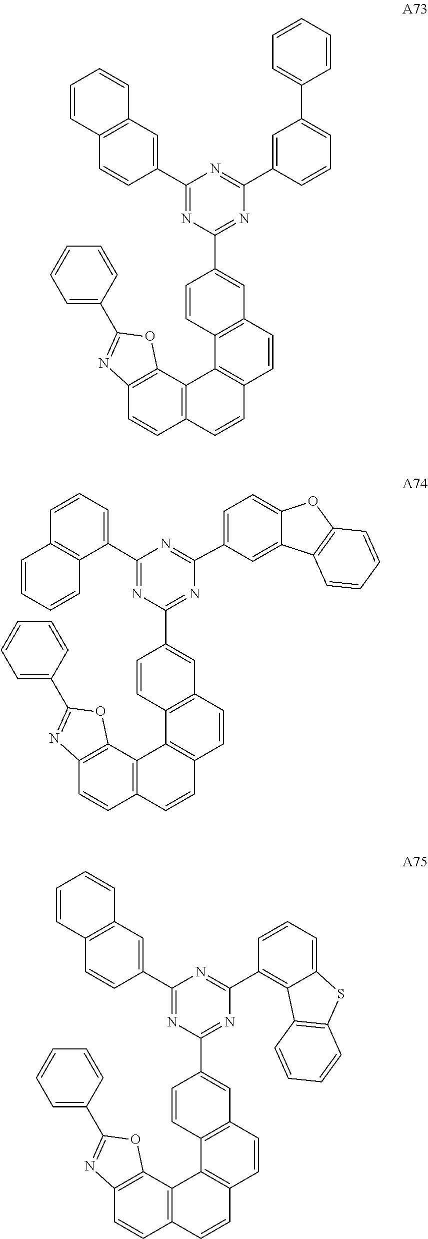 [SMILES: c1ccc(-c2cccc(-c3nc(-c4ccc5ccccc5c4)nc(-c4ccc5c(ccc6ccc7ccc8nc(-c9ccccc9)oc8c7c65)c4)n3)c2)cc1 ; c1ccc(-c2nc3ccc4ccc5ccc6cc(-c7nc(-c8ccc9oc%10ccccc%10c9c8)nc(-c8cccc9ccccc89)n7)ccc6c5c4c3o2)cc1 ; c1ccc(-c2nc3ccc4ccc5ccc6cc(-c7nc(-c8ccc9ccccc9c8)nc(-c8cccc9sc%10ccccc%10c89)n7)ccc6c5c4c3o2)cc1]

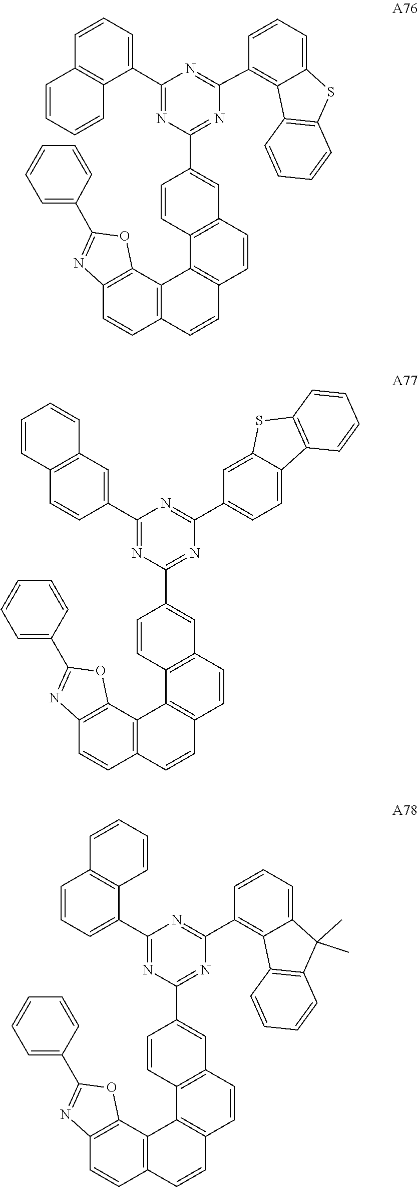 [SMILES: c1ccc(-c2nc3ccc4ccc5ccc6cc(-c7nc(-c8cccc9ccccc89)nc(-c8cccc9sc%10ccccc%10c89)n7)ccc6c5c4c3o2)cc1 ; c1ccc(-c2nc3ccc4ccc5ccc6cc(-c7nc(-c8ccc9ccccc9c8)nc(-c8ccc9c(c8)sc8ccccc89)n7)ccc6c5c4c3o2)cc1 ; CC1(C)c2ccccc2-c2c(-c3nc(-c4ccc5c(ccc6ccc7ccc8nc(-c9ccccc9)oc8c7c65)c4)nc(-c4cccc5ccccc45)n3)cccc21]

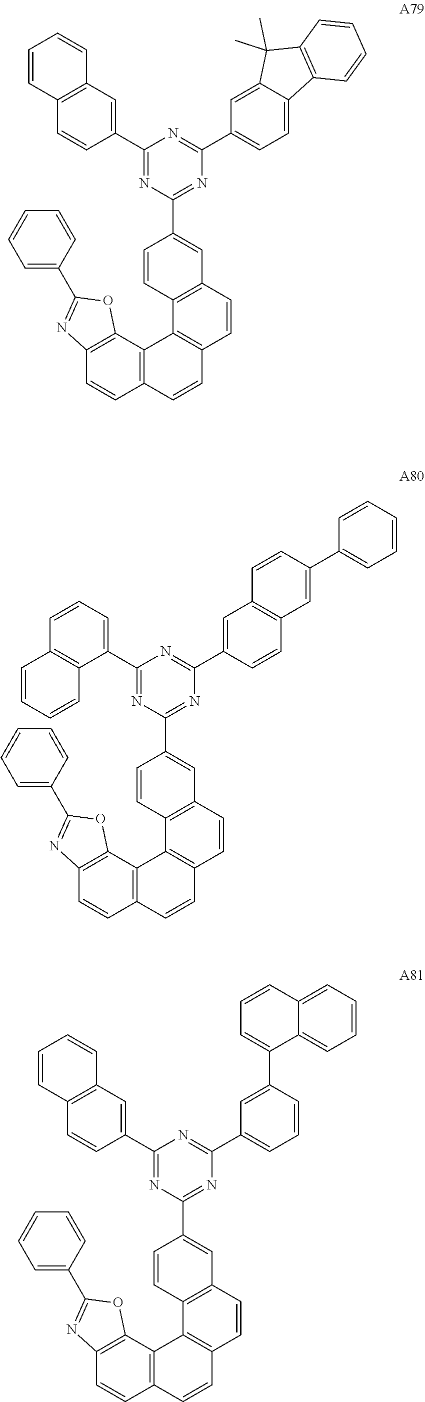 [SMILES: CC1(C)c2ccccc2-c2ccc(-c3nc(-c4ccc5ccccc5c4)nc(-c4ccc5c(ccc6ccc7ccc8nc(-c9ccccc9)oc8c7c65)c4)n3)cc21 ; c1ccc(-c2ccc3cc(-c4nc(-c5ccc6c(ccc7ccc8ccc9nc(-c%10ccccc%10)oc9c8c76)c5)nc(-c5cccc6ccccc56)n4)ccc3c2)cc1 ; c1ccc(-c2nc3ccc4ccc5ccc6cc(-c7nc(-c8cccc(-c9cccc%10ccccc9%10)c8)nc(-c8ccc9ccccc9c8)n7)ccc6c5c4c3o2)cc1]

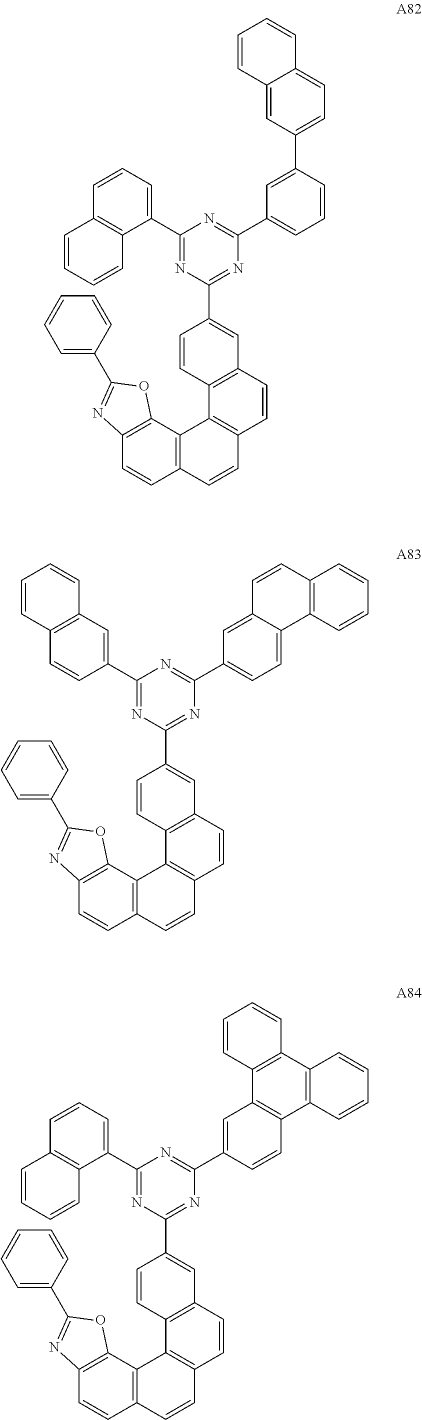 [SMILES: c1ccc(-c2nc3ccc4ccc5ccc6cc(-c7nc(-c8cccc(-c9ccc%10ccccc%10c9)c8)nc(-c8cccc9ccccc89)n7)ccc6c5c4c3o2)cc1 ; c1ccc(-c2nc3ccc4ccc5ccc6cc(-c7nc(-c8ccc9ccccc9c8)nc(-c8ccc9c(ccc%10ccccc%109)c8)n7)ccc6c5c4c3o2)cc1 ; c1ccc(-c2nc3ccc4ccc5ccc6cc(-c7nc(-c8ccc9c%10ccccc%10c%10ccccc%10c9c8)nc(-c8cccc9ccccc89)n7)ccc6c5c4c3o2)cc1]

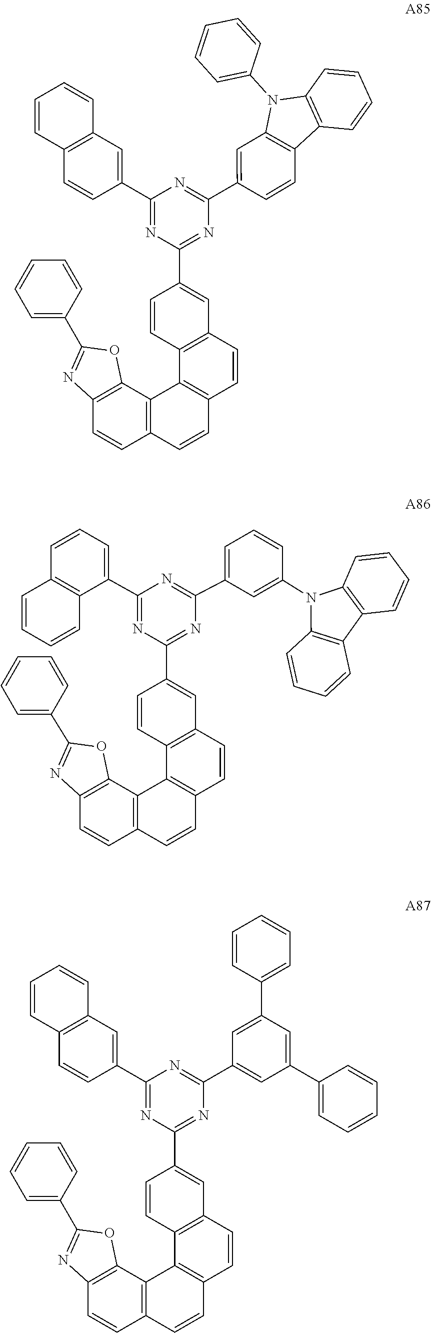 [SMILES: c1ccc(-c2nc3ccc4ccc5ccc6cc(-c7nc(-c8ccc9ccccc9c8)nc(-c8ccc9c%10ccccc%10n(-c%10ccccc%10)c9c8)n7)ccc6c5c4c3o2)cc1 ; c1ccc(-c2nc3ccc4ccc5ccc6cc(-c7nc(-c8cccc(-n9c%10ccccc%10c%10ccccc%109)c8)nc(-c8cccc9ccccc89)n7)ccc6c5c4c3o2)cc1 ; c1ccc(-c2cc(-c3ccccc3)cc(-c3nc(-c4ccc5ccccc5c4)nc(-c4ccc5c(ccc6ccc7ccc8nc(-c9ccccc9)oc8c7c65)c4)n3)c2)cc1]

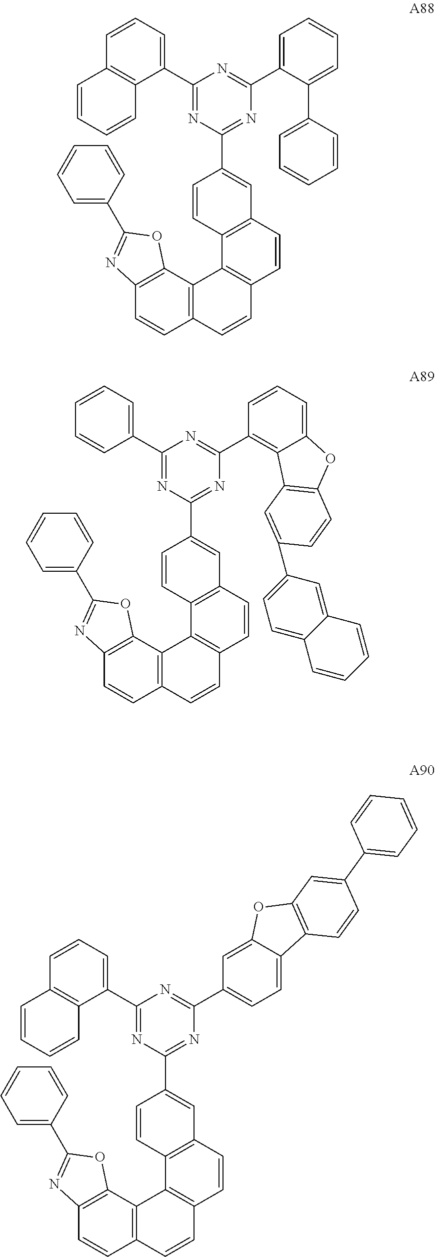 [SMILES: c1ccc(-c2nc3ccc4ccc5ccc6cc(-c7nc(-c8ccccc8-c8ccccc8)nc(-c8cccc9ccccc89)n7)ccc6c5c4c3o2)cc1 ; c1ccc(-c2nc(-c3ccc4c(ccc5ccc6ccc7nc(-c8ccccc8)oc7c6c54)c3)nc(-c3cccc4oc5ccc(-c6ccc7ccccc7c6)cc5c34)n2)cc1 ; c1ccc(-c2ccc3c(c2)oc2cc(-c4nc(-c5ccc6c(ccc7ccc8ccc9nc(-c%10ccccc%10)oc9c8c76)c5)nc(-c5cccc6ccccc56)n4)ccc23)cc1]

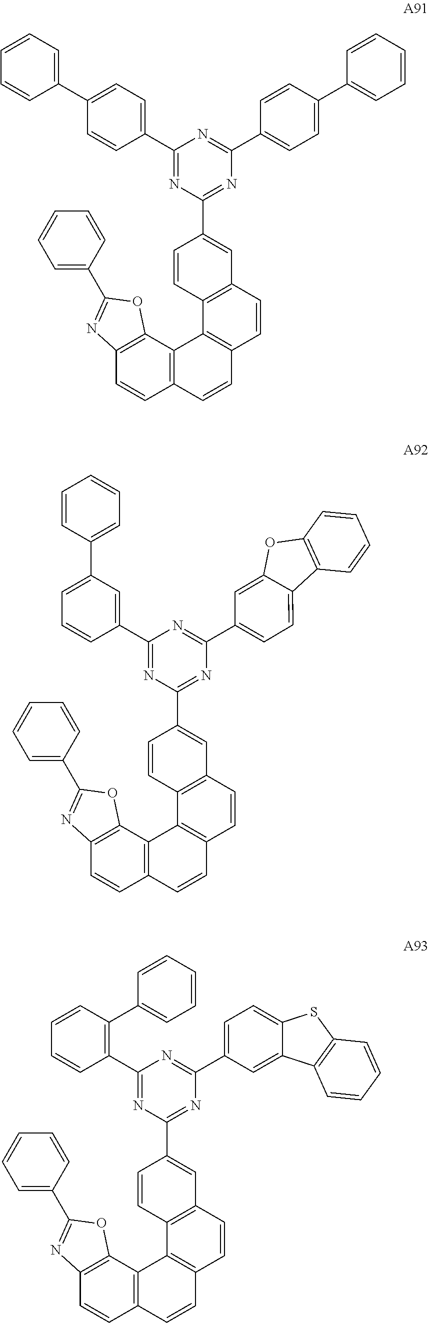 [SMILES: c1ccc(-c2ccc(-c3nc(-c4ccc(-c5ccccc5)cc4)nc(-c4ccc5c(ccc6ccc7ccc8nc(-c9ccccc9)oc8c7c65)c4)n3)cc2)cc1 ; c1ccc(-c2cccc(-c3nc(-c4ccc5c(ccc6ccc7ccc8nc(-c9ccccc9)oc8c7c65)c4)nc(-c4ccc5c(c4)oc4ccccc45)n3)c2)cc1 ; c1ccc(-c2nc3ccc4ccc5ccc6cc(-c7nc(-c8ccc9sc%10ccccc%10c9c8)nc(-c8ccccc8-c8ccccc8)n7)ccc6c5c4c3o2)cc1]

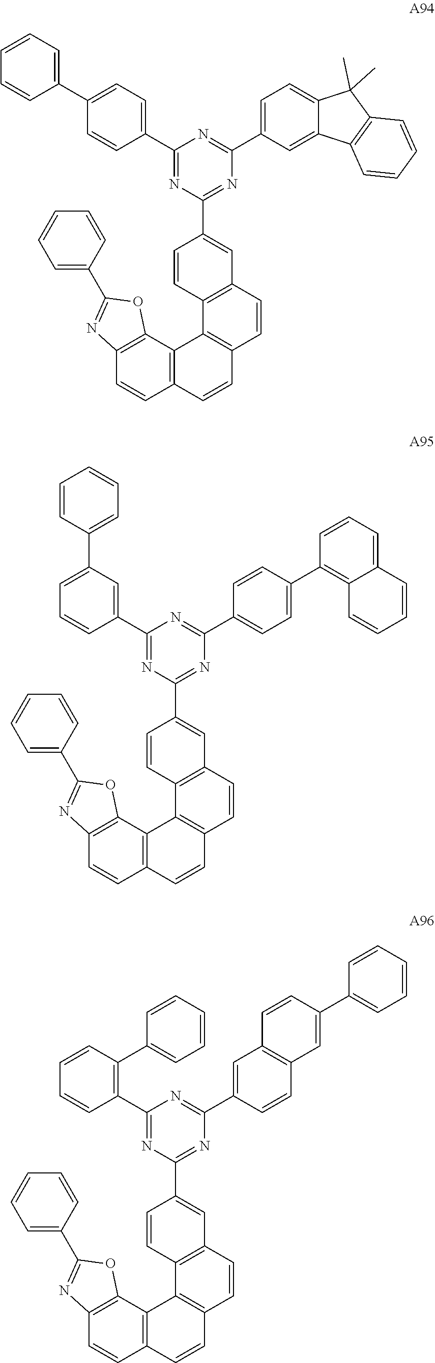 [SMILES: CC1(C)c2ccccc2-c2cc(-c3nc(-c4ccc(-c5ccccc5)cc4)nc(-c4ccc5c(ccc6ccc7ccc8nc(-c9ccccc9)oc8c7c65)c4)n3)ccc21 ; c1ccc(-c2cccc(-c3nc(-c4ccc(-c5cccc6ccccc56)cc4)nc(-c4ccc5c(ccc6ccc7ccc8nc(-c9ccccc9)oc8c7c65)c4)n3)c2)cc1 ; c1ccc(-c2ccc3cc(-c4nc(-c5ccc6c(ccc7ccc8ccc9nc(-c%10ccccc%10)oc9c8c76)c5)nc(-c5ccccc5-c5ccccc5)n4)ccc3c2)cc1]

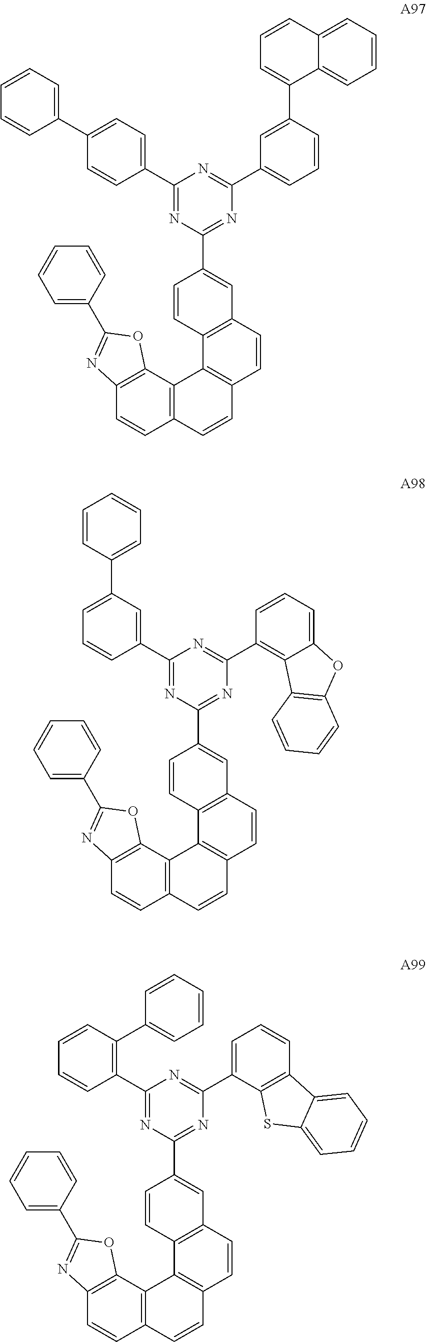 [SMILES: c1ccc(-c2ccc(-c3nc(-c4cccc(-c5cccc6ccccc56)c4)nc(-c4ccc5c(ccc6ccc7ccc8nc(-c9ccccc9)oc8c7c65)c4)n3)cc2)cc1 ; c1ccc(-c2cccc(-c3nc(-c4ccc5c(ccc6ccc7ccc8nc(-c9ccccc9)oc8c7c65)c4)nc(-c4cccc5oc6ccccc6c45)n3)c2)cc1 ; c1ccc(-c2nc3ccc4ccc5ccc6cc(-c7nc(-c8ccccc8-c8ccccc8)nc(-c8cccc9c8sc8ccccc89)n7)ccc6c5c4c3o2)cc1]

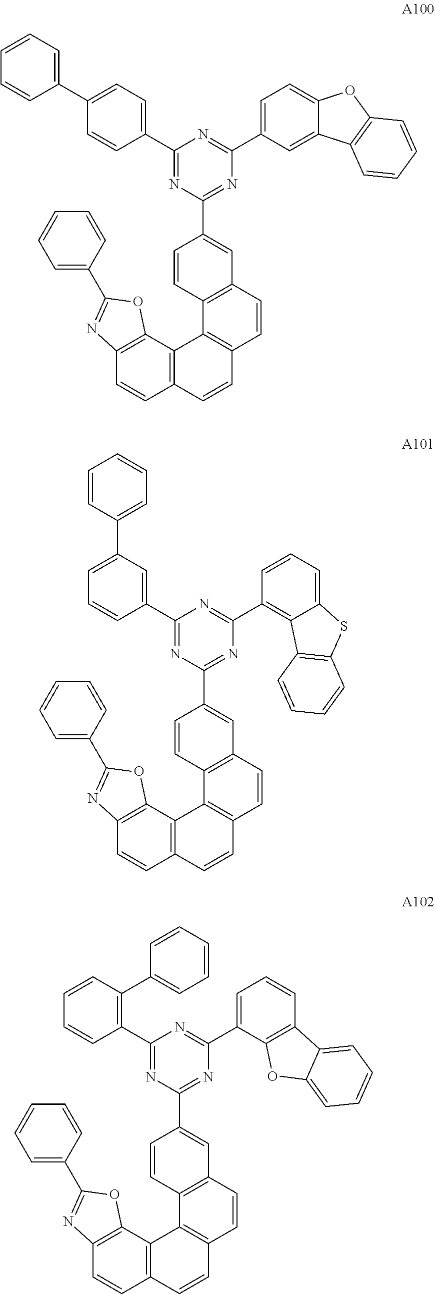 [SMILES: c1ccc(-c2ccc(-c3nc(-c4ccc5c(ccc6ccc7ccc8nc(-c9ccccc9)oc8c7c65)c4)nc(-c4ccc5oc6ccccc6c5c4)n3)cc2)cc1 ; c1ccc(-c2cccc(-c3nc(-c4ccc5c(ccc6ccc7ccc8nc(-c9ccccc9)oc8c7c65)c4)nc(-c4cccc5sc6ccccc6c45)n3)c2)cc1 ; c1ccc(-c2nc3ccc4ccc5ccc6cc(-c7nc(-c8ccccc8-c8ccccc8)nc(-c8cccc9c8oc8ccccc89)n7)ccc6c5c4c3o2)cc1]

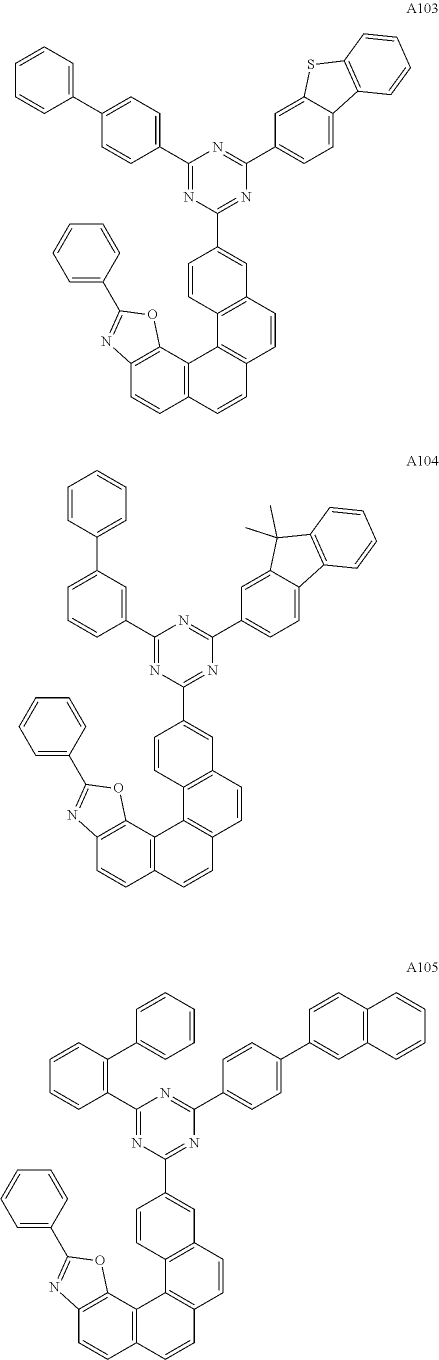 [SMILES: c1ccc(-c2ccc(-c3nc(-c4ccc5c(ccc6ccc7ccc8nc(-c9ccccc9)oc8c7c65)c4)nc(-c4ccc5c(c4)sc4ccccc45)n3)cc2)cc1 ; CC1(C)c2ccccc2-c2ccc(-c3nc(-c4cccc(-c5ccccc5)c4)nc(-c4ccc5c(ccc6ccc7ccc8nc(-c9ccccc9)oc8c7c65)c4)n3)cc21 ; c1ccc(-c2nc3ccc4ccc5ccc6cc(-c7nc(-c8ccc(-c9ccc%10ccccc%10c9)cc8)nc(-c8ccccc8-c8ccccc8)n7)ccc6c5c4c3o2)cc1]

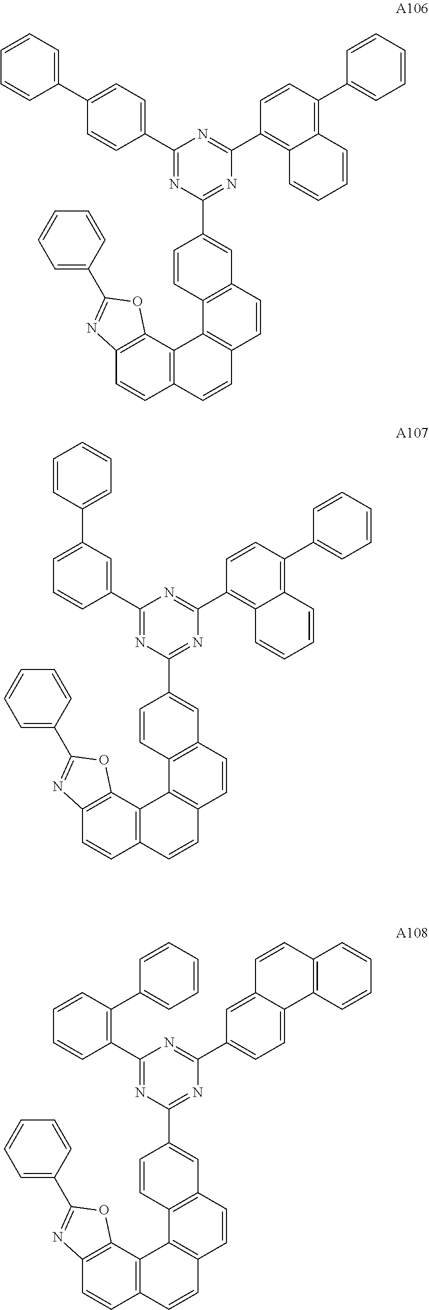 [SMILES: c1ccc(-c2ccc(-c3nc(-c4ccc5c(ccc6ccc7ccc8nc(-c9ccccc9)oc8c7c65)c4)nc(-c4ccc(-c5ccccc5)c5ccccc45)n3)cc2)cc1 ; c1ccc(-c2cccc(-c3nc(-c4ccc5c(ccc6ccc7ccc8nc(-c9ccccc9)oc8c7c65)c4)nc(-c4ccc(-c5ccccc5)c5ccccc45)n3)c2)cc1 ; c1ccc(-c2nc3ccc4ccc5ccc6cc(-c7nc(-c8ccc9c(ccc%10ccccc%109)c8)nc(-c8ccccc8-c8ccccc8)n7)ccc6c5c4c3o2)cc1]

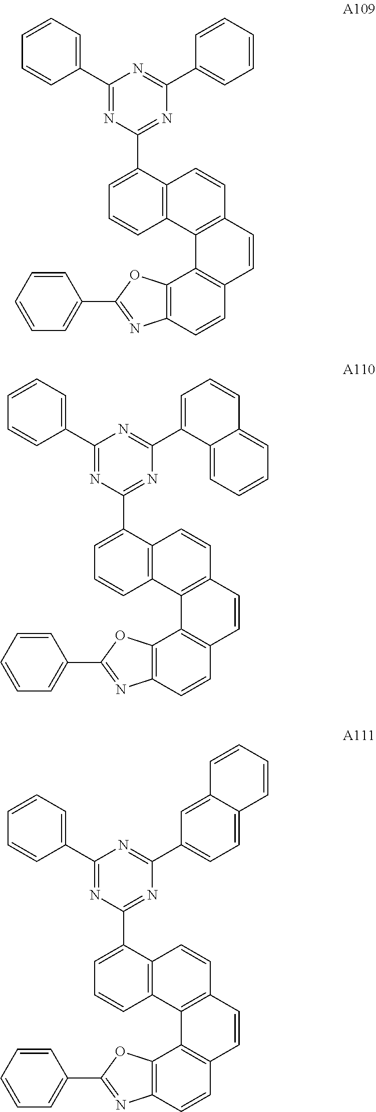

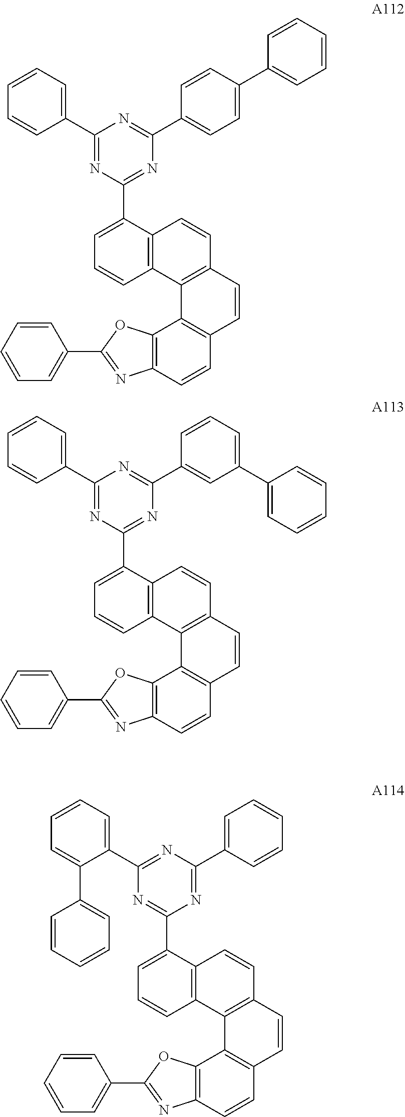

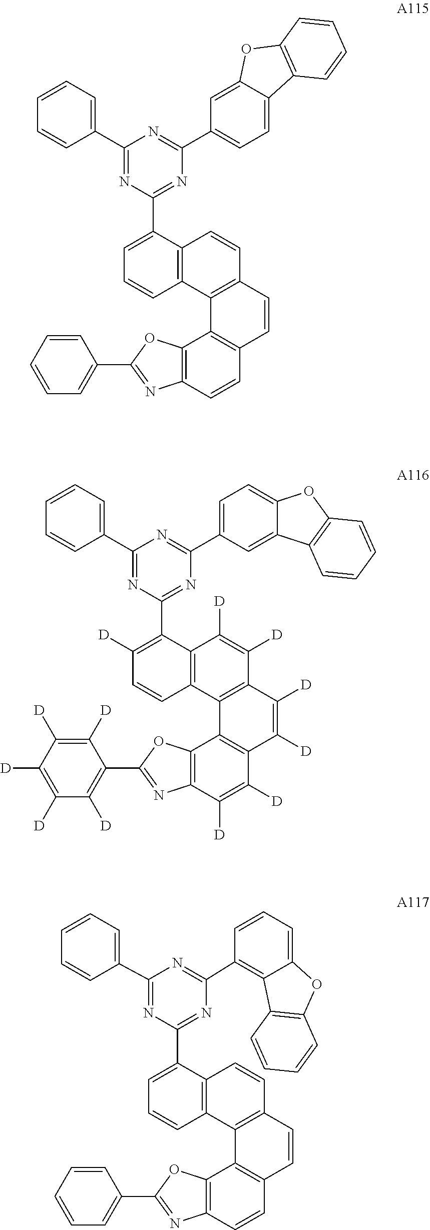 [SMILES: c1ccc(-c2nc(-c3ccc4c(c3)oc3ccccc34)nc(-c3cccc4c3ccc3ccc5ccc6nc(-c7ccccc7)oc6c5c34)n2)cc1 ; [2H]c1ccc2c(c([2H])c([2H])c3c([2H])c([2H])c4c([2H])c([2H])c5nc(-c6c([2H])c([2H])c([2H])c([2H])c6[2H])oc5c4c32)c1-c1nc(-c2ccccc2)nc(-c2ccc3oc4ccccc4c3c2)n1 ; c1ccc(-c2nc(-c3cccc4c3ccc3ccc5ccc6nc(-c7ccccc7)oc6c5c34)nc(-c3cccc4oc5ccccc5c34)n2)cc1]

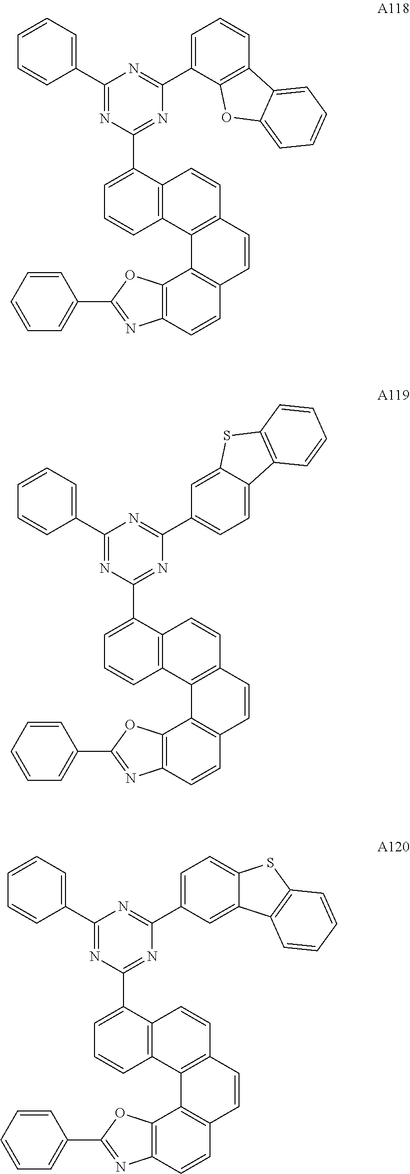 [SMILES: c1ccc(-c2nc(-c3cccc4c3ccc3ccc5ccc6nc(-c7ccccc7)oc6c5c34)nc(-c3cccc4c3oc3ccccc34)n2)cc1 ; c1ccc(-c2nc(-c3ccc4c(c3)sc3ccccc34)nc(-c3cccc4c3ccc3ccc5ccc6nc(-c7ccccc7)oc6c5c34)n2)cc1 ; c1ccc(-c2nc(-c3ccc4sc5ccccc5c4c3)nc(-c3cccc4c3ccc3ccc5ccc6nc(-c7ccccc7)oc6c5c34)n2)cc1]

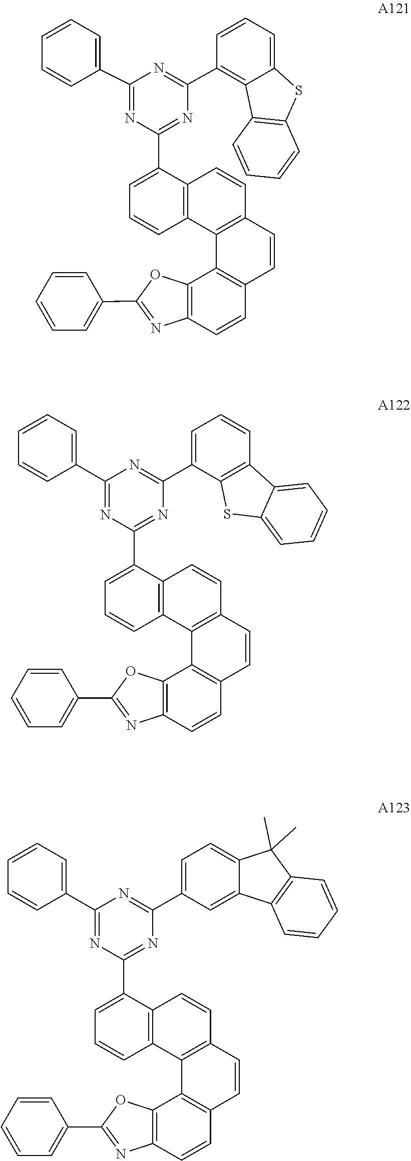 [SMILES: c1ccc(-c2nc(-c3cccc4c3ccc3ccc5ccc6nc(-c7ccccc7)oc6c5c34)nc(-c3cccc4sc5ccccc5c34)n2)cc1 ; c1ccc(-c2nc(-c3cccc4c3ccc3ccc5ccc6nc(-c7ccccc7)oc6c5c34)nc(-c3cccc4c3sc3ccccc34)n2)cc1 ; CC1(C)c2ccccc2-c2cc(-c3nc(-c4ccccc4)nc(-c4cccc5c4ccc4ccc6ccc7nc(-c8ccccc8)oc7c6c45)n3)ccc21]

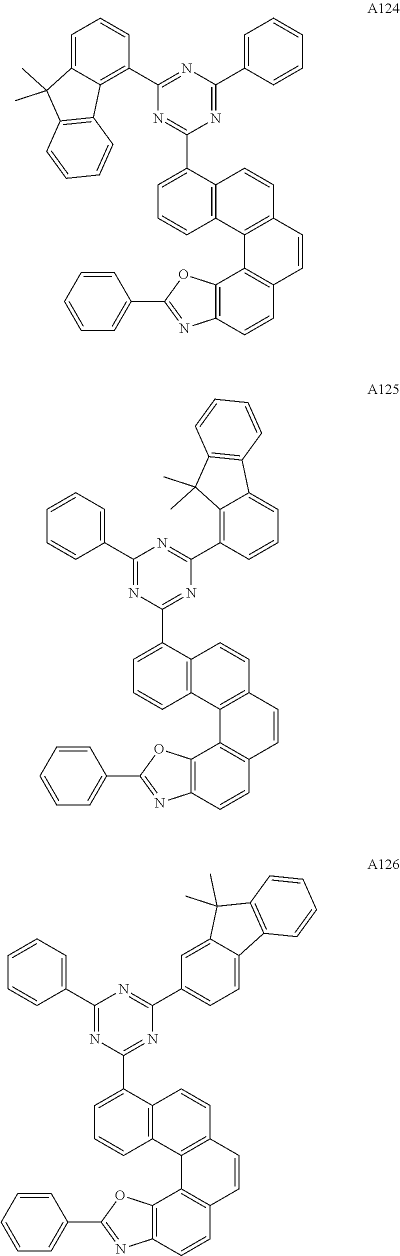 [SMILES: CC1(C)c2ccccc2-c2c(-c3nc(-c4ccccc4)nc(-c4cccc5c4ccc4ccc6ccc7nc(-c8ccccc8)oc7c6c45)n3)cccc21 ; CC1(C)c2ccccc2-c2cccc(-c3nc(-c4ccccc4)nc(-c4cccc5c4ccc4ccc6ccc7nc(-c8ccccc8)oc7c6c45)n3)c21 ; CC1(C)c2ccccc2-c2ccc(-c3nc(-c4ccccc4)nc(-c4cccc5c4ccc4ccc6ccc7nc(-c8ccccc8)oc7c6c45)n3)cc21]

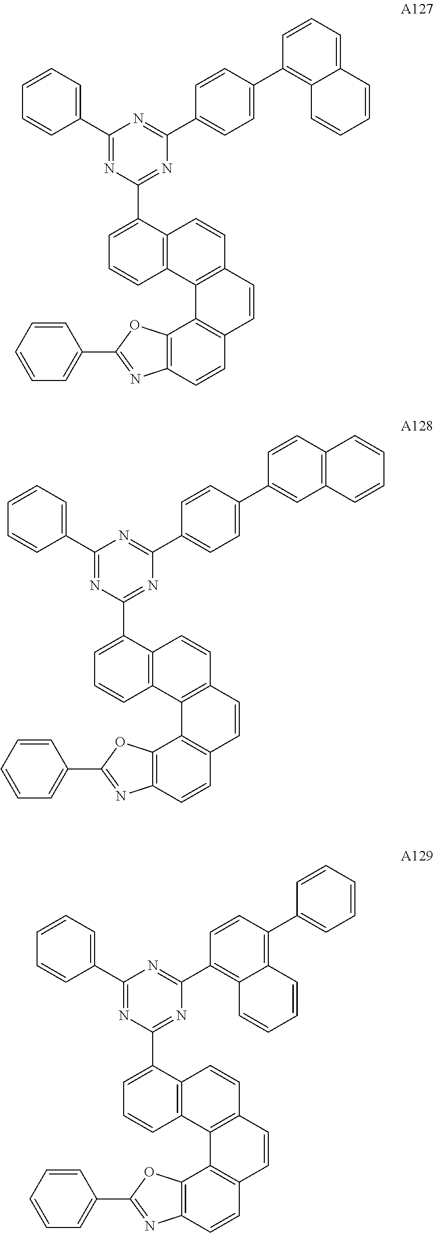 [SMILES: c1ccc(-c2nc(-c3ccc(-c4cccc5ccccc45)cc3)nc(-c3cccc4c3ccc3ccc5ccc6nc(-c7ccccc7)oc6c5c34)n2)cc1 ; c1ccc(-c2nc(-c3ccc(-c4ccc5ccccc5c4)cc3)nc(-c3cccc4c3ccc3ccc5ccc6nc(-c7ccccc7)oc6c5c34)n2)cc1 ; c1ccc(-c2nc(-c3ccc(-c4ccccc4)c4ccccc34)nc(-c3cccc4c3ccc3ccc5ccc6nc(-c7ccccc7)oc6c5c34)n2)cc1]

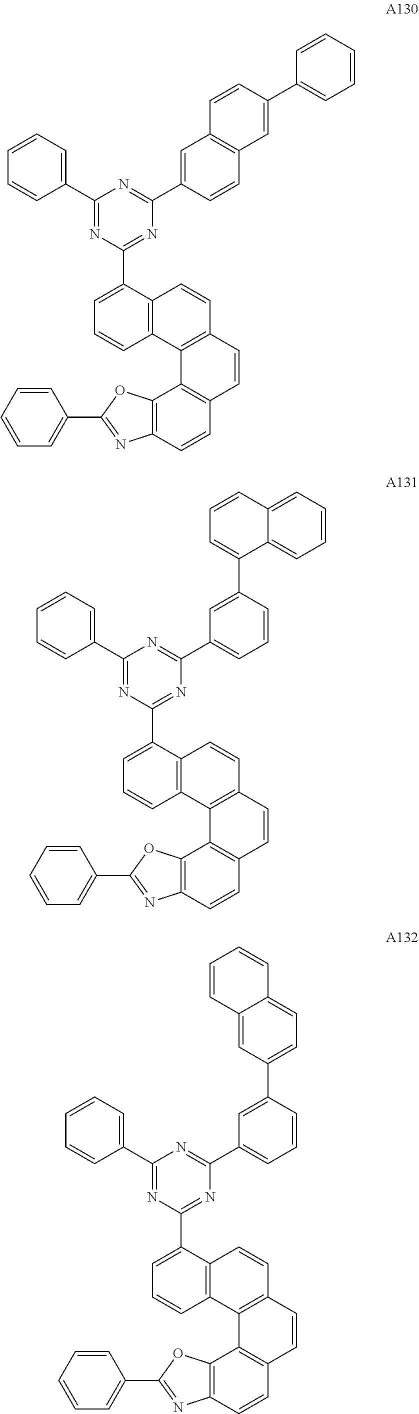 [SMILES: c1ccc(-c2ccc3cc(-c4nc(-c5ccccc5)nc(-c5cccc6c5ccc5ccc7ccc8nc(-c9ccccc9)oc8c7c56)n4)ccc3c2)cc1 ; c1ccc(-c2nc(-c3cccc(-c4cccc5ccccc45)c3)nc(-c3cccc4c3ccc3ccc5ccc6nc(-c7ccccc7)oc6c5c34)n2)cc1 ; c1ccc(-c2nc(-c3cccc(-c4ccc5ccccc5c4)c3)nc(-c3cccc4c3ccc3ccc5ccc6nc(-c7ccccc7)oc6c5c34)n2)cc1]

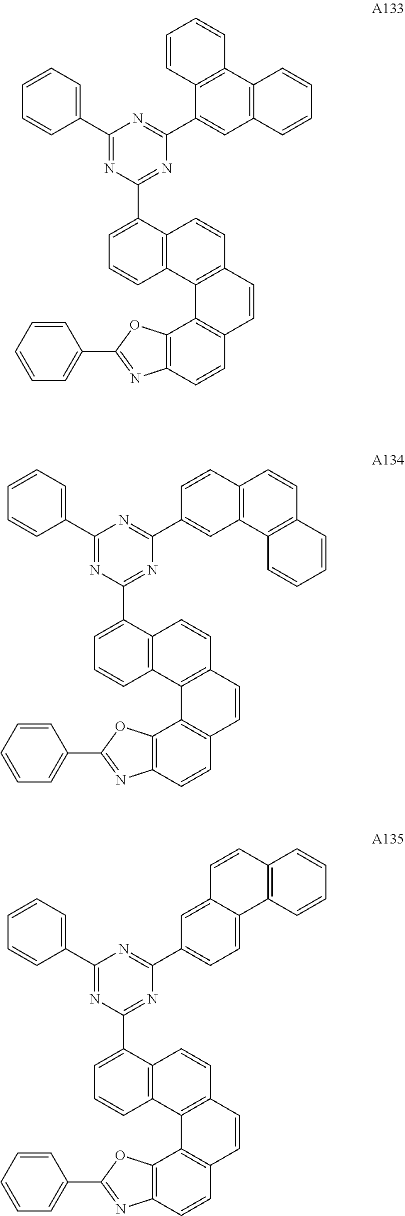 [SMILES: c1ccc(-c2nc(-c3cc4ccccc4c4ccccc34)nc(-c3cccc4c3ccc3ccc5ccc6nc(-c7ccccc7)oc6c5c34)n2)cc1 ; c1ccc(-c2nc(-c3ccc4ccc5ccccc5c4c3)nc(-c3cccc4c3ccc3ccc5ccc6nc(-c7ccccc7)oc6c5c34)n2)cc1 ; c1ccc(-c2nc(-c3ccc4c(ccc5ccccc54)c3)nc(-c3cccc4c3ccc3ccc5ccc6nc(-c7ccccc7)oc6c5c34)n2)cc1]

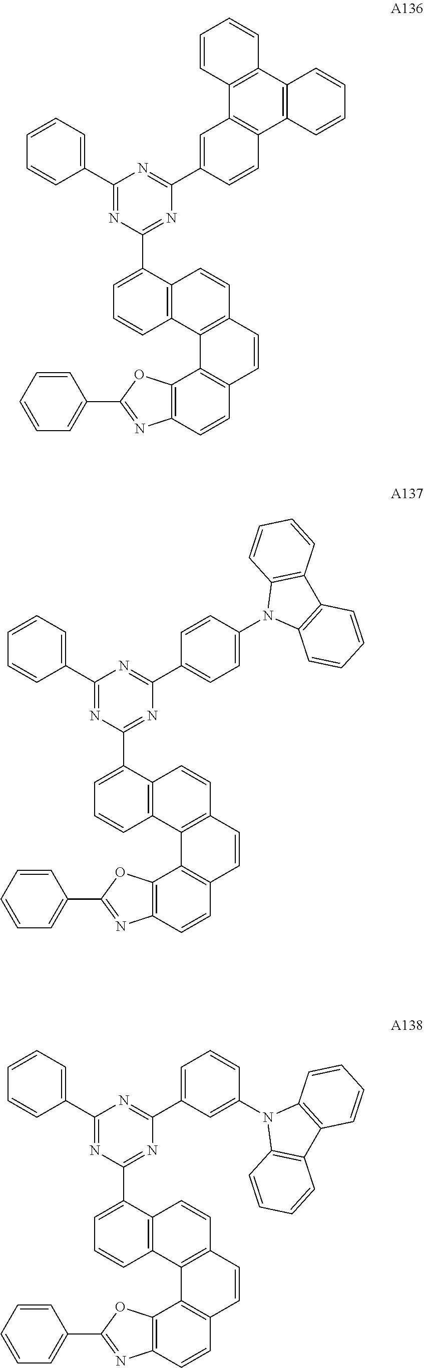 [SMILES: c1ccc(-c2nc(-c3ccc4c5ccccc5c5ccccc5c4c3)nc(-c3cccc4c3ccc3ccc5ccc6nc(-c7ccccc7)oc6c5c34)n2)cc1 ; c1ccc(-c2nc(-c3ccc(-n4c5ccccc5c5ccccc54)cc3)nc(-c3cccc4c3ccc3ccc5ccc6nc(-c7ccccc7)oc6c5c34)n2)cc1 ; c1ccc(-c2nc(-c3cccc(-n4c5ccccc5c5ccccc54)c3)nc(-c3cccc4c3ccc3ccc5ccc6nc(-c7ccccc7)oc6c5c34)n2)cc1]

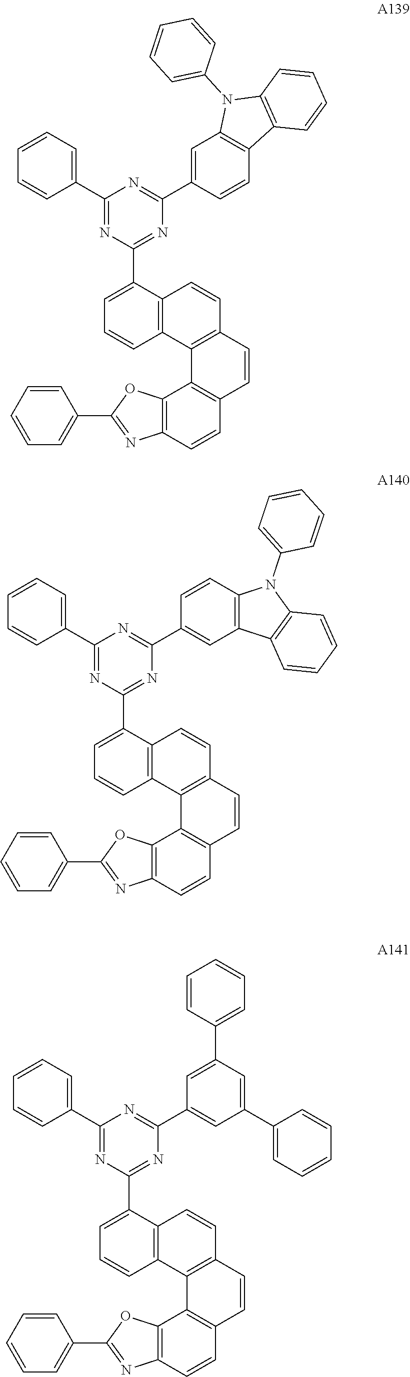 [SMILES: c1ccc(-c2nc(-c3ccc4c5ccccc5n(-c5ccccc5)c4c3)nc(-c3cccc4c3ccc3ccc5ccc6nc(-c7ccccc7)oc6c5c34)n2)cc1 ; c1ccc(-c2nc(-c3ccc4c(c3)c3ccccc3n4-c3ccccc3)nc(-c3cccc4c3ccc3ccc5ccc6nc(-c7ccccc7)oc6c5c34)n2)cc1 ; c1ccc(-c2cc(-c3ccccc3)cc(-c3nc(-c4ccccc4)nc(-c4cccc5c4ccc4ccc6ccc7nc(-c8ccccc8)oc7c6c45)n3)c2)cc1]

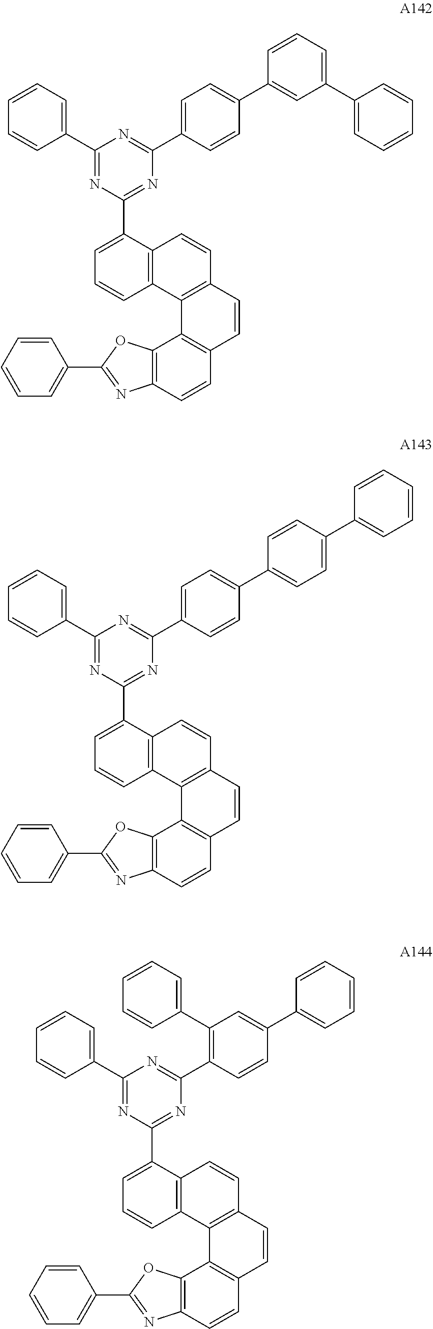 [SMILES: c1ccc(-c2cccc(-c3ccc(-c4nc(-c5ccccc5)nc(-c5cccc6c5ccc5ccc7ccc8nc(-c9ccccc9)oc8c7c56)n4)cc3)c2)cc1 ; c1ccc(-c2ccc(-c3ccc(-c4nc(-c5ccccc5)nc(-c5cccc6c5ccc5ccc7ccc8nc(-c9ccccc9)oc8c7c56)n4)cc3)cc2)cc1 ; c1ccc(-c2ccc(-c3nc(-c4ccccc4)nc(-c4cccc5c4ccc4ccc6ccc7nc(-c8ccccc8)oc7c6c45)n3)c(-c3ccccc3)c2)cc1]

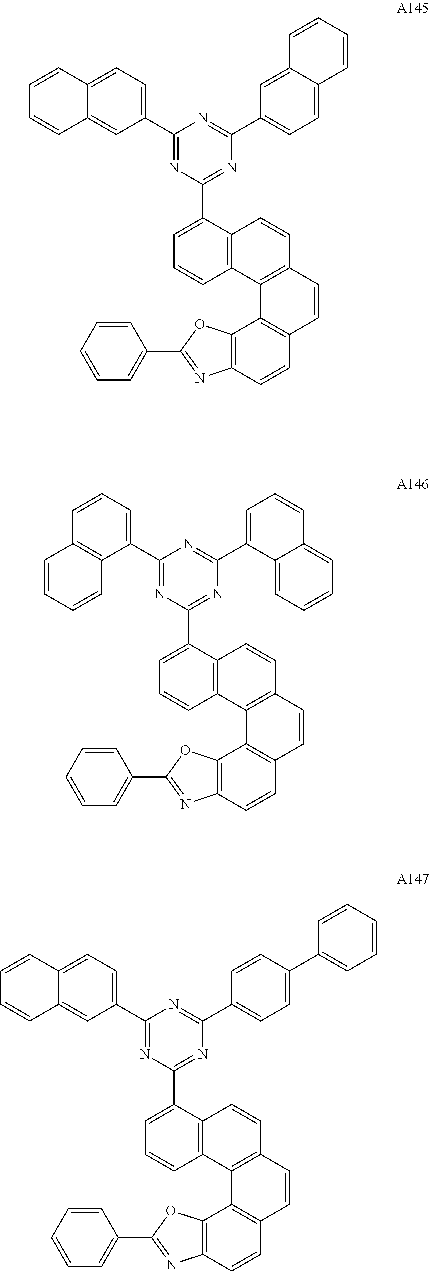 [SMILES: c1ccc(-c2nc3ccc4ccc5ccc6c(-c7nc(-c8ccc9ccccc9c8)nc(-c8ccc9ccccc9c8)n7)cccc6c5c4c3o2)cc1 ; c1ccc(-c2nc3ccc4ccc5ccc6c(-c7nc(-c8cccc9ccccc89)nc(-c8cccc9ccccc89)n7)cccc6c5c4c3o2)cc1 ; c1ccc(-c2ccc(-c3nc(-c4ccc5ccccc5c4)nc(-c4cccc5c4ccc4ccc6ccc7nc(-c8ccccc8)oc7c6c45)n3)cc2)cc1]

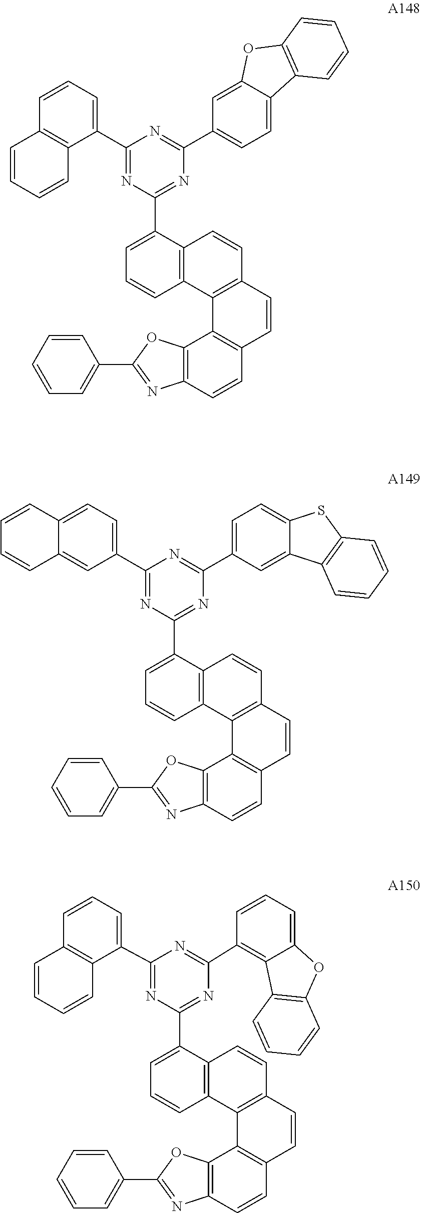 [SMILES: c1ccc(-c2nc3ccc4ccc5ccc6c(-c7nc(-c8ccc9c(c8)oc8ccccc89)nc(-c8cccc9ccccc89)n7)cccc6c5c4c3o2)cc1 ; c1ccc(-c2nc3ccc4ccc5ccc6c(-c7nc(-c8ccc9ccccc9c8)nc(-c8ccc9sc%10ccccc%10c9c8)n7)cccc6c5c4c3o2)cc1 ; c1ccc(-c2nc3ccc4ccc5ccc6c(-c7nc(-c8cccc9ccccc89)nc(-c8cccc9oc%10ccccc%10c89)n7)cccc6c5c4c3o2)cc1]

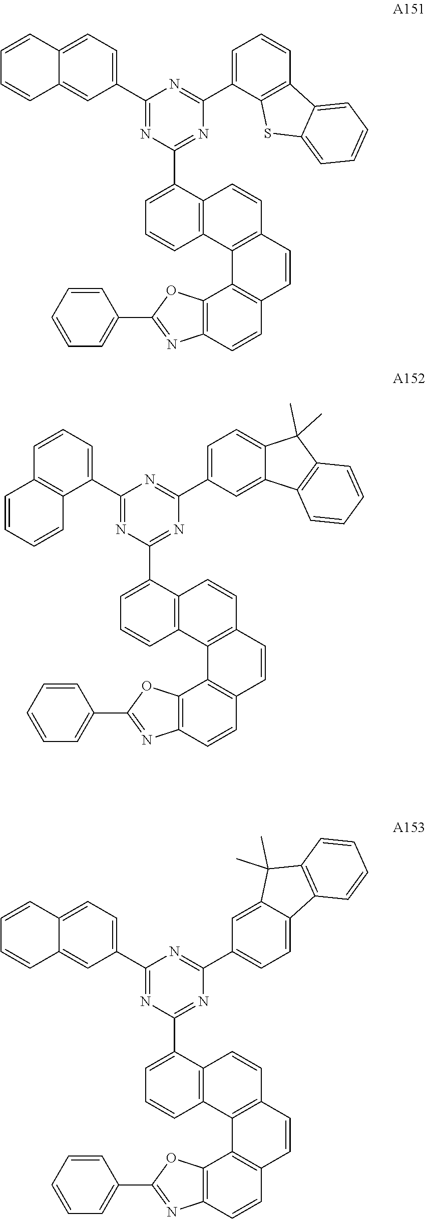 [SMILES: c1ccc(-c2nc3ccc4ccc5ccc6c(-c7nc(-c8ccc9ccccc9c8)nc(-c8cccc9c8sc8ccccc89)n7)cccc6c5c4c3o2)cc1 ; CC1(C)c2ccccc2-c2cc(-c3nc(-c4cccc5ccccc45)nc(-c4cccc5c4ccc4ccc6ccc7nc(-c8ccccc8)oc7c6c45)n3)ccc21 ; CC1(C)c2ccccc2-c2ccc(-c3nc(-c4ccc5ccccc5c4)nc(-c4cccc5c4ccc4ccc6ccc7nc(-c8ccccc8)oc7c6c45)n3)cc21]

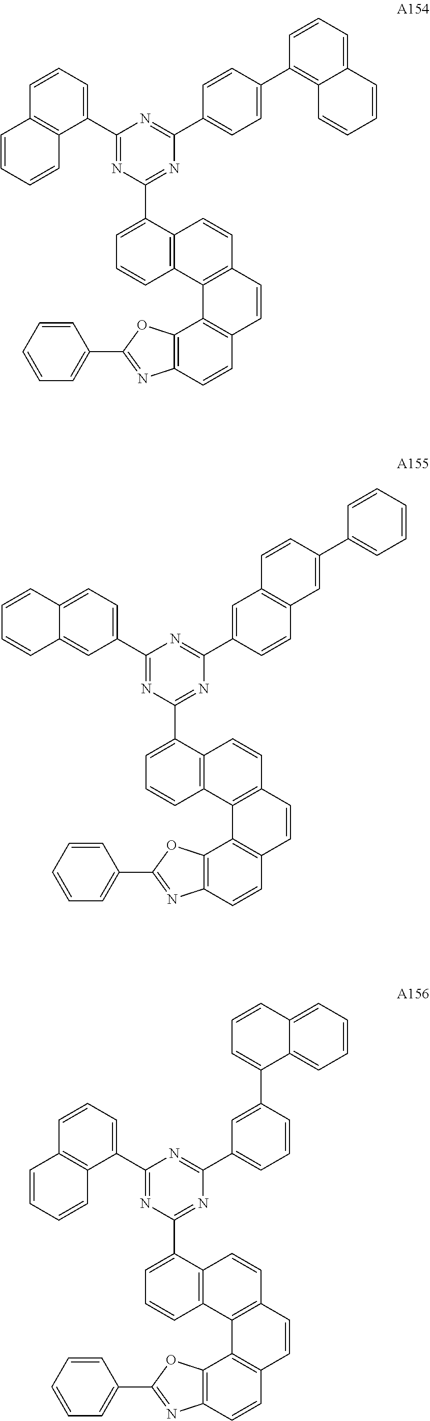 [SMILES: c1ccc(-c2nc3ccc4ccc5ccc6c(-c7nc(-c8ccc(-c9cccc%10ccccc9%10)cc8)nc(-c8cccc9ccccc89)n7)cccc6c5c4c3o2)cc1 ; c1ccc(-c2ccc3cc(-c4nc(-c5ccc6ccccc6c5)nc(-c5cccc6c5ccc5ccc7ccc8nc(-c9ccccc9)oc8c7c56)n4)ccc3c2)cc1 ; c1ccc(-c2nc3ccc4ccc5ccc6c(-c7nc(-c8cccc(-c9cccc%10ccccc9%10)c8)nc(-c8cccc9ccccc89)n7)cccc6c5c4c3o2)cc1]

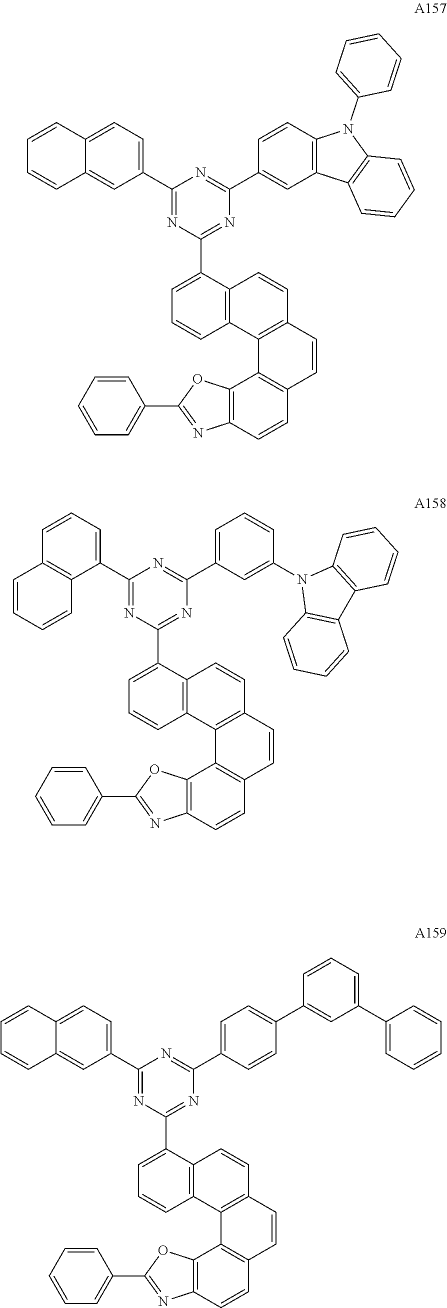 [SMILES: c1ccc(-c2nc3ccc4ccc5ccc6c(-c7nc(-c8ccc9ccccc9c8)nc(-c8ccc9c(c8)c8ccccc8n9-c8ccccc8)n7)cccc6c5c4c3o2)cc1 ; c1ccc(-c2nc3ccc4ccc5ccc6c(-c7nc(-c8cccc(-n9c%10ccccc%10c%10ccccc%109)c8)nc(-c8cccc9ccccc89)n7)cccc6c5c4c3o2)cc1 ; c1ccc(-c2cccc(-c3ccc(-c4nc(-c5ccc6ccccc6c5)nc(-c5cccc6c5ccc5ccc7ccc8nc(-c9ccccc9)oc8c7c56)n4)cc3)c2)cc1]

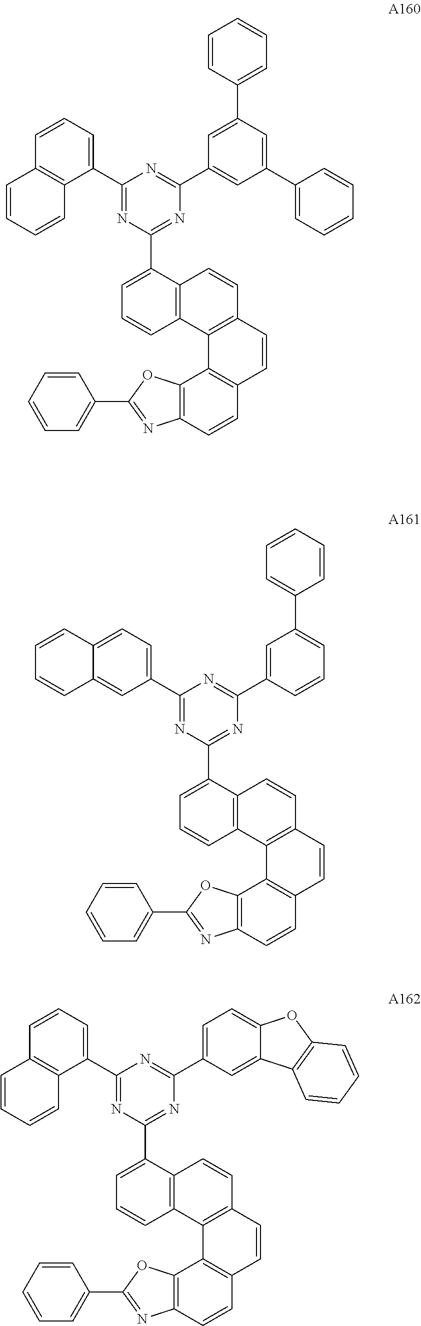 [SMILES: c1ccc(-c2cc(-c3ccccc3)cc(-c3nc(-c4cccc5ccccc45)nc(-c4cccc5c4ccc4ccc6ccc7nc(-c8ccccc8)oc7c6c45)n3)c2)cc1 ; c1ccc(-c2cccc(-c3nc(-c4ccc5ccccc5c4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8ccc9oc%10ccccc%10c9c8)nc(-c8cccc9ccccc89)n7)cccc6c5c4c3o2)cc1]

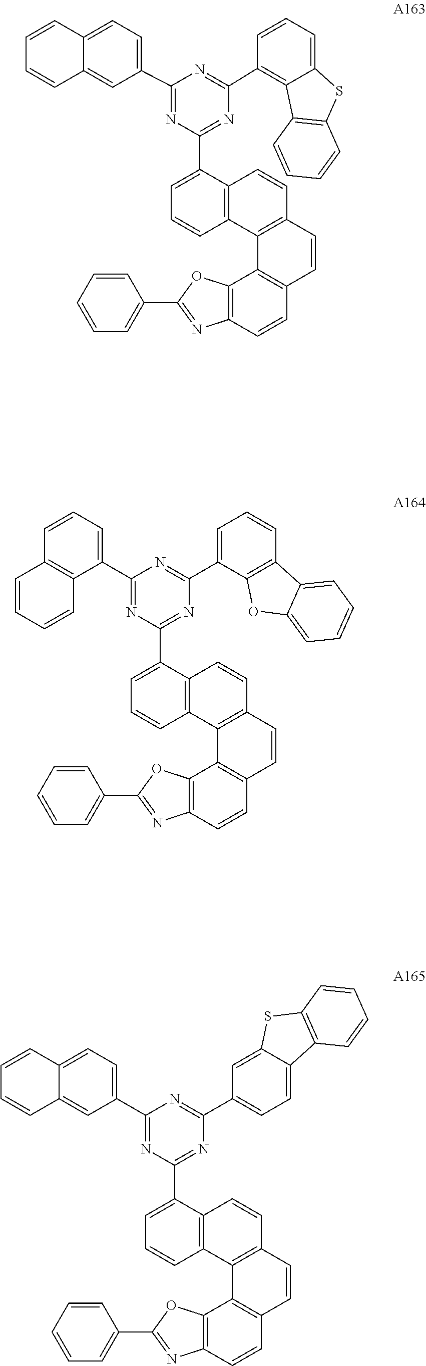 [SMILES: c1ccc(-c2nc3ccc4ccc5ccc6c(-c7nc(-c8ccc9ccccc9c8)nc(-c8cccc9sc%10ccccc%10c89)n7)cccc6c5c4c3o2)cc1 ; c1ccc(-c2nc3ccc4ccc5ccc6c(-c7nc(-c8cccc9ccccc89)nc(-c8cccc9c8oc8ccccc89)n7)cccc6c5c4c3o2)cc1 ; c1ccc(-c2nc3ccc4ccc5ccc6c(-c7nc(-c8ccc9ccccc9c8)nc(-c8ccc9c(c8)sc8ccccc89)n7)cccc6c5c4c3o2)cc1]

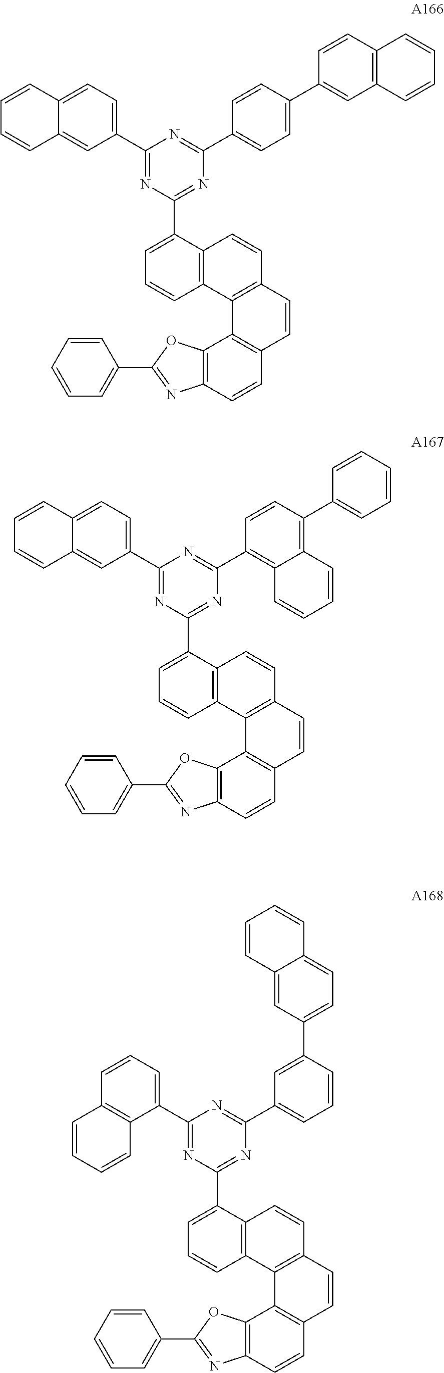 [SMILES: c1ccc(-c2nc3ccc4ccc5ccc6c(-c7nc(-c8ccc(-c9ccc%10ccccc%10c9)cc8)nc(-c8ccc9ccccc9c8)n7)cccc6c5c4c3o2)cc1 ; c1ccc(-c2nc3ccc4ccc5ccc6c(-c7nc(-c8ccc9ccccc9c8)nc(-c8ccc(-c9ccccc9)c9ccccc89)n7)cccc6c5c4c3o2)cc1 ; c1ccc(-c2nc3ccc4ccc5ccc6c(-c7nc(-c8cccc(-c9ccc%10ccccc%10c9)c8)nc(-c8cccc9ccccc89)n7)cccc6c5c4c3o2)cc1]

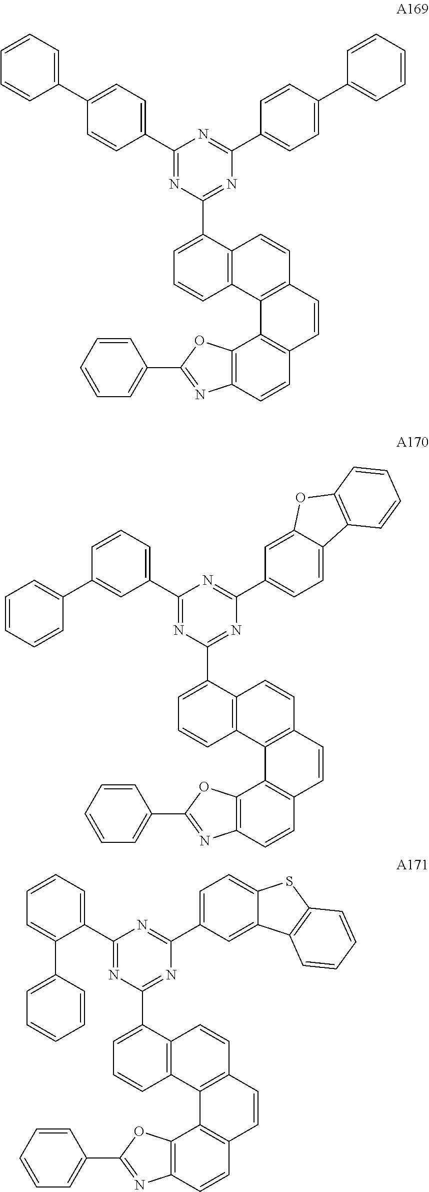

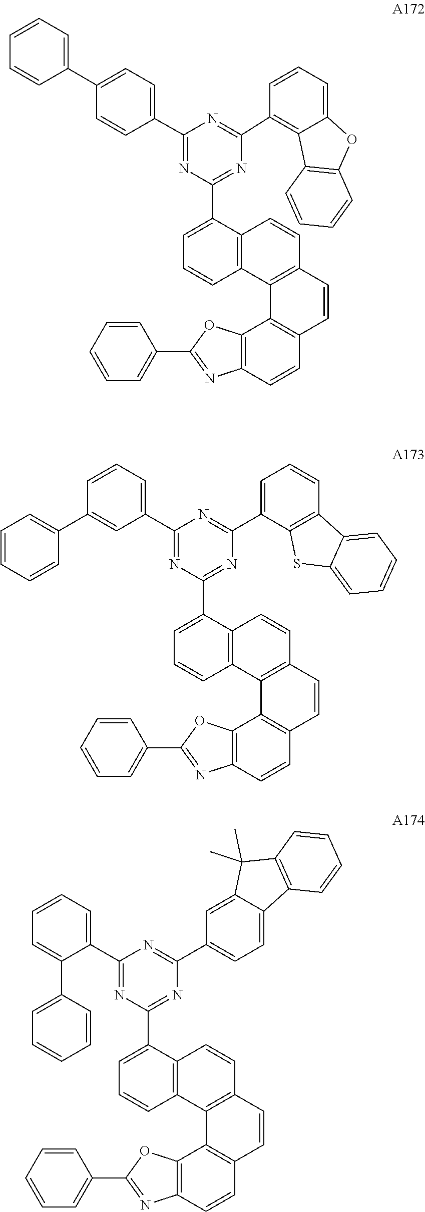 [SMILES: c1ccc(-c2ccc(-c3nc(-c4cccc5c4ccc4ccc6ccc7nc(-c8ccccc8)oc7c6c45)nc(-c4cccc5oc6ccccc6c45)n3)cc2)cc1 ; c1ccc(-c2cccc(-c3nc(-c4cccc5c4ccc4ccc6ccc7nc(-c8ccccc8)oc7c6c45)nc(-c4cccc5c4sc4ccccc45)n3)c2)cc1 ; CC1(C)c2ccccc2-c2ccc(-c3nc(-c4ccccc4-c4ccccc4)nc(-c4cccc5c4ccc4ccc6ccc7nc(-c8ccccc8)oc7c6c45)n3)cc21]

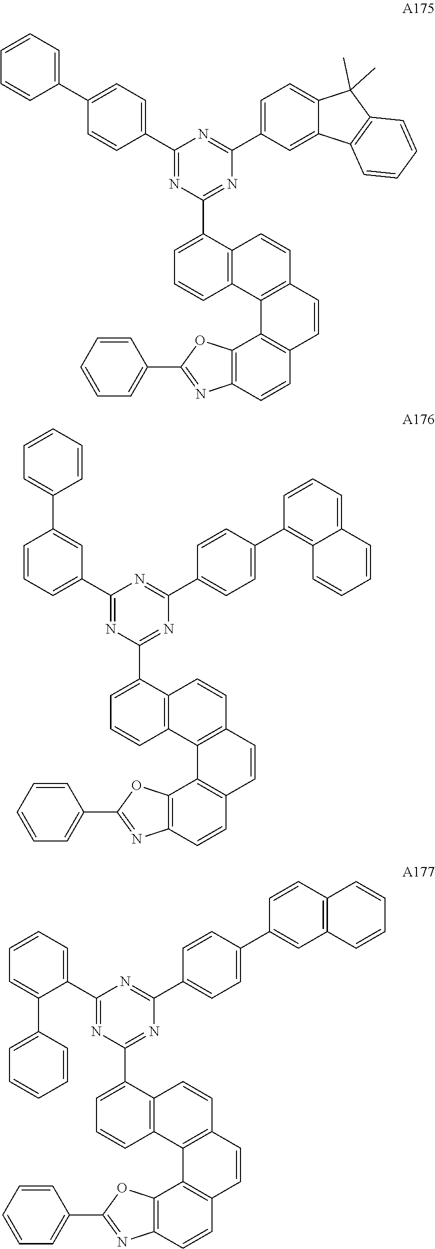 [SMILES: CC1(C)c2ccccc2-c2cc(-c3nc(-c4ccc(-c5ccccc5)cc4)nc(-c4cccc5c4ccc4ccc6ccc7nc(-c8ccccc8)oc7c6c45)n3)ccc21 ; c1ccc(-c2cccc(-c3nc(-c4ccc(-c5cccc6ccccc56)cc4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8ccc(-c9ccc%10ccccc%10c9)cc8)nc(-c8ccccc8-c8ccccc8)n7)cccc6c5c4c3o2)cc1]

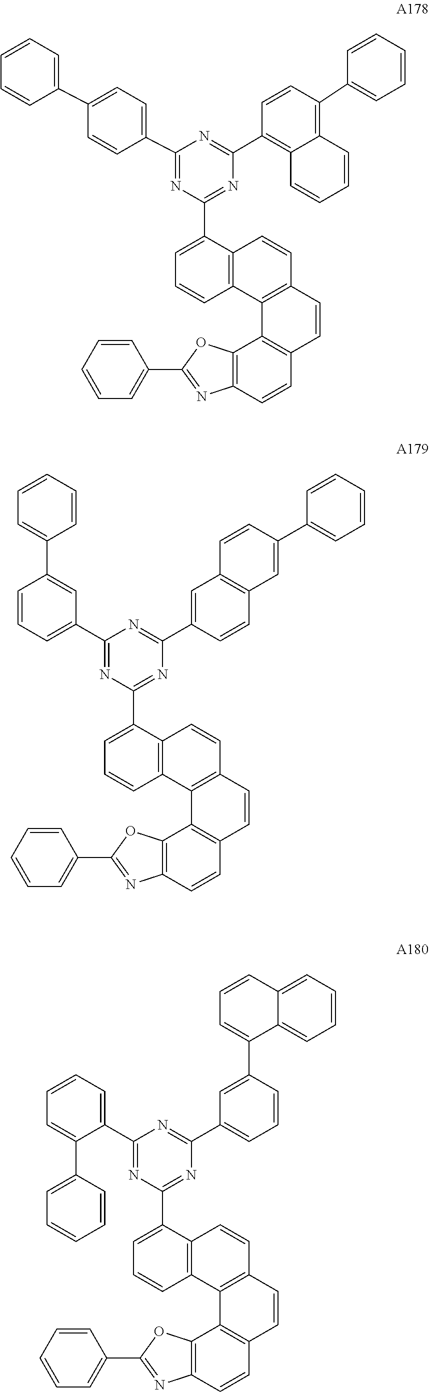 [SMILES: c1ccc(-c2ccc(-c3nc(-c4ccc(-c5ccccc5)c5ccccc45)nc(-c4cccc5c4ccc4ccc6ccc7nc(-c8ccccc8)oc7c6c45)n3)cc2)cc1 ; c1ccc(-c2cccc(-c3nc(-c4ccc5cc(-c6ccccc6)ccc5c4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8cccc(-c9cccc%10ccccc9%10)c8)nc(-c8ccccc8-c8ccccc8)n7)cccc6c5c4c3o2)cc1]

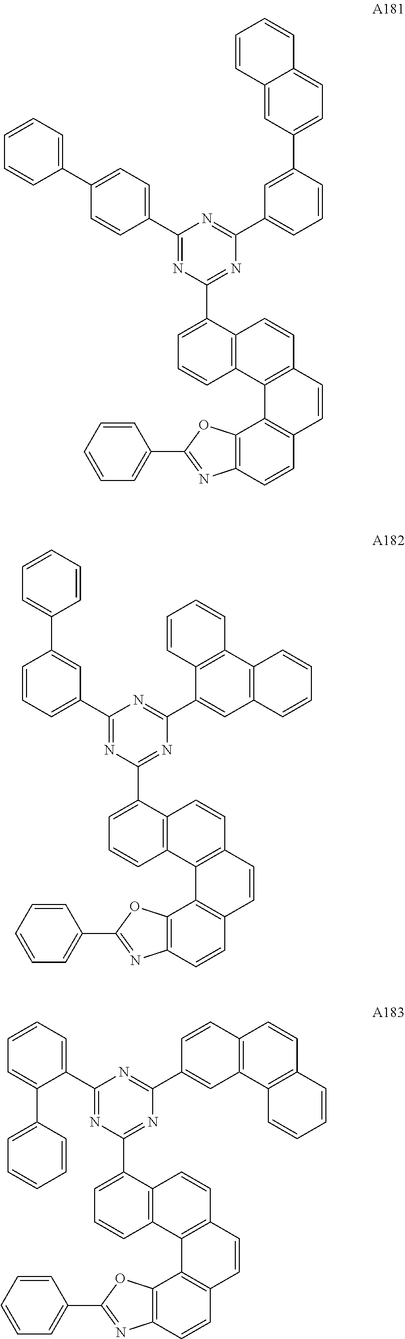 [SMILES: c1ccc(-c2ccc(-c3nc(-c4cccc(-c5ccc6ccccc6c5)c4)nc(-c4cccc5c4ccc4ccc6ccc7nc(-c8ccccc8)oc7c6c45)n3)cc2)cc1 ; c1ccc(-c2cccc(-c3nc(-c4cc5ccccc5c5ccccc45)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8ccc9ccc%10ccccc%10c9c8)nc(-c8ccccc8-c8ccccc8)n7)cccc6c5c4c3o2)cc1]

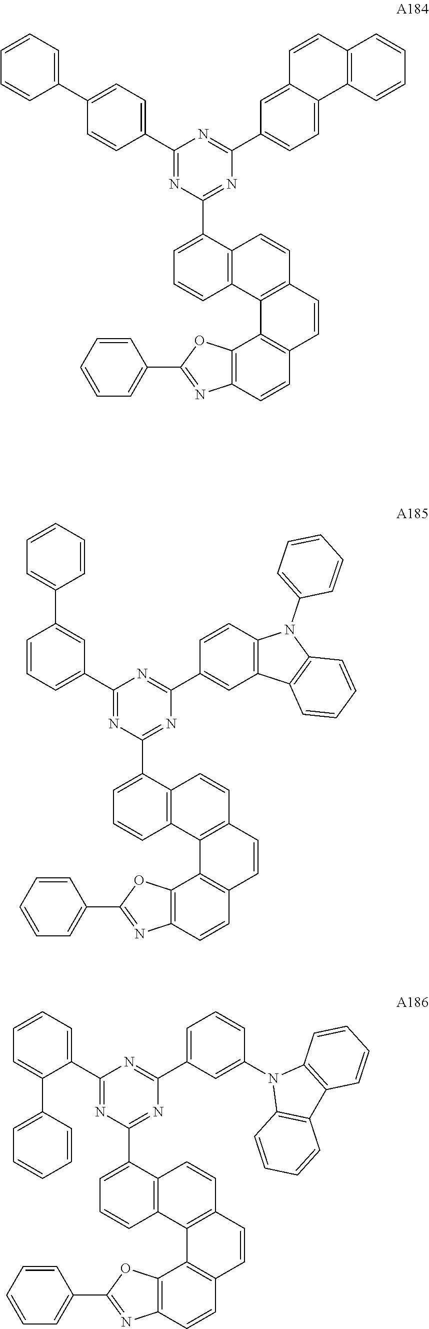 [SMILES: c1ccc(-c2ccc(-c3nc(-c4ccc5c(ccc6ccccc65)c4)nc(-c4cccc5c4ccc4ccc6ccc7nc(-c8ccccc8)oc7c6c45)n3)cc2)cc1 ; c1ccc(-c2cccc(-c3nc(-c4ccc5c(c4)c4ccccc4n5-c4ccccc4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8cccc(-n9c%10ccccc%10c%10ccccc%109)c8)nc(-c8ccccc8-c8ccccc8)n7)cccc6c5c4c3o2)cc1]

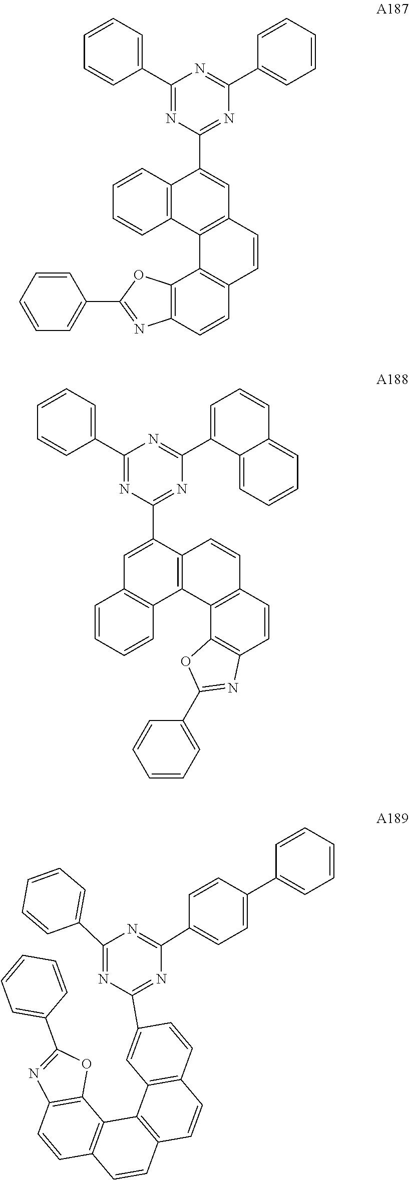 [SMILES: c1ccc(-c2nc(-c3ccccc3)nc(-c3cc4ccc5ccc6nc(-c7ccccc7)oc6c5c4c4ccccc34)n2)cc1 ; c1ccc(-c2nc(-c3cccc4ccccc34)nc(-c3cc4ccccc4c4c3ccc3ccc5nc(-c6ccccc6)oc5c34)n2)cc1 ; c1ccc(-c2ccc(-c3nc(-c4ccccc4)nc(-c4ccc5ccc6ccc7ccc8nc(-c9ccccc9)oc8c7c6c5c4)n3)cc2)cc1]

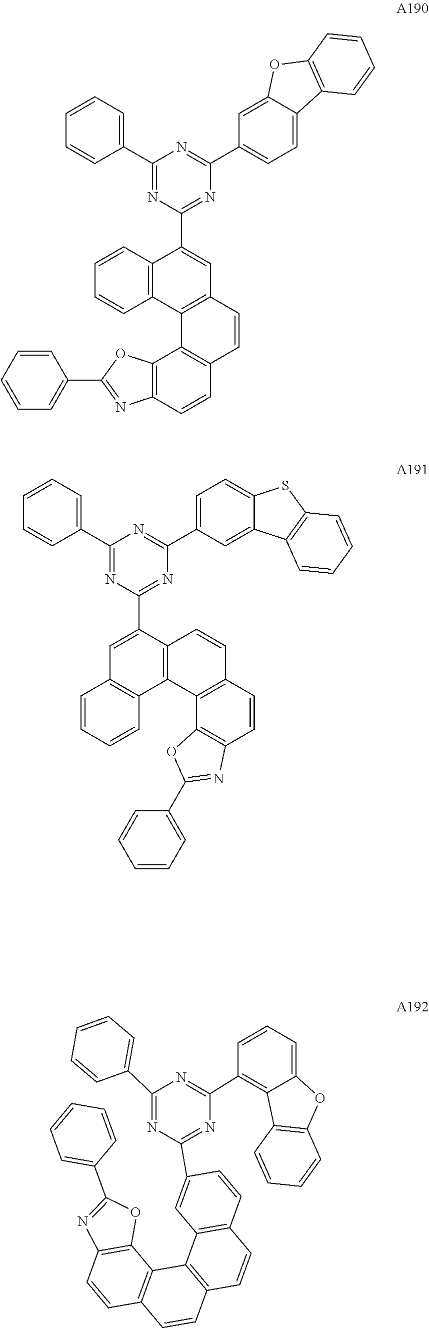 [SMILES: c1ccc(-c2nc(-c3ccc4c(c3)oc3ccccc34)nc(-c3cc4ccc5ccc6nc(-c7ccccc7)oc6c5c4c4ccccc34)n2)cc1 ; c1ccc(-c2nc(-c3ccc4sc5ccccc5c4c3)nc(-c3cc4ccccc4c4c3ccc3ccc5nc(-c6ccccc6)oc5c34)n2)cc1 ; c1ccc(-c2nc(-c3ccc4ccc5ccc6ccc7nc(-c8ccccc8)oc7c6c5c4c3)nc(-c3cccc4oc5ccccc5c34)n2)cc1]

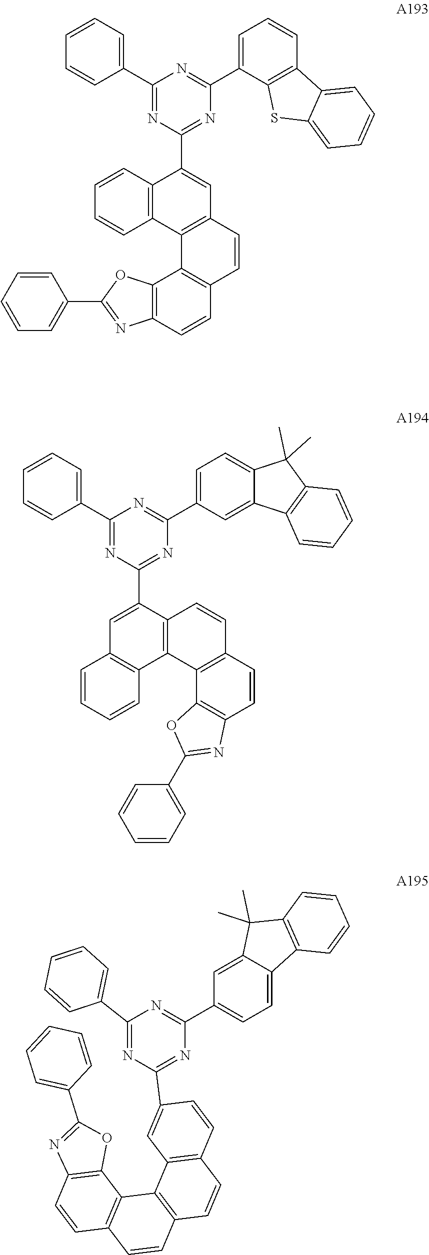 [SMILES: c1ccc(-c2nc(-c3cc4ccc5ccc6nc(-c7ccccc7)oc6c5c4c4ccccc34)nc(-c3cccc4c3sc3ccccc34)n2)cc1 ; CC1(C)c2ccccc2-c2cc(-c3nc(-c4ccccc4)nc(-c4cc5ccccc5c5c4ccc4ccc6nc(-c7ccccc7)oc6c45)n3)ccc21 ; CC1(C)c2ccccc2-c2ccc(-c3nc(-c4ccccc4)nc(-c4ccc5ccc6ccc7ccc8nc(-c9ccccc9)oc8c7c6c5c4)n3)cc21]

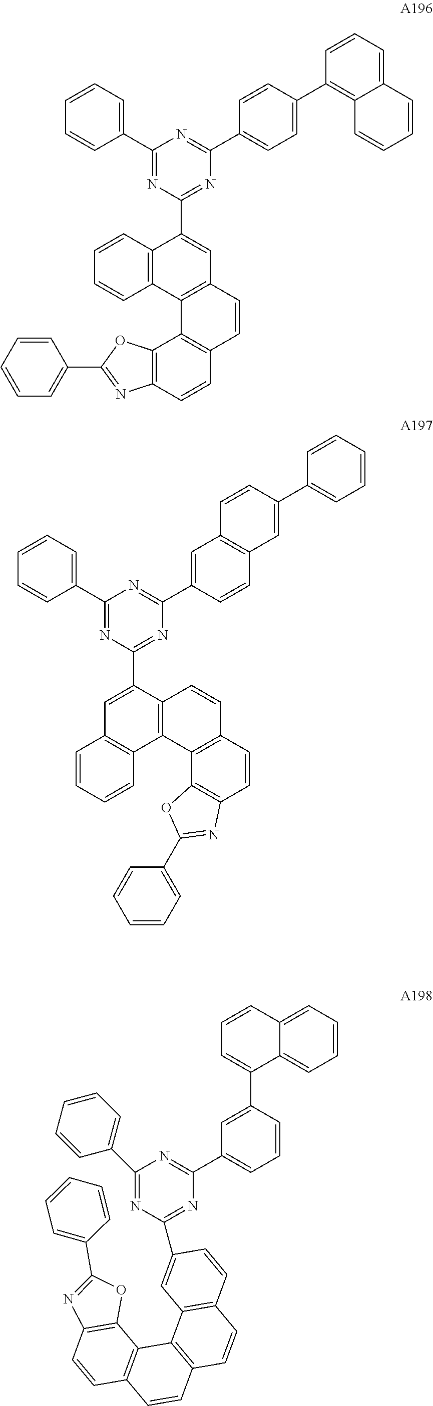 [SMILES: c1ccc(-c2nc(-c3ccc(-c4cccc5ccccc45)cc3)nc(-c3cc4ccc5ccc6nc(-c7ccccc7)oc6c5c4c4ccccc34)n2)cc1 ; c1ccc(-c2ccc3cc(-c4nc(-c5ccccc5)nc(-c5cc6ccccc6c6c5ccc5ccc7nc(-c8ccccc8)oc7c56)n4)ccc3c2)cc1 ; c1ccc(-c2nc(-c3cccc(-c4cccc5ccccc45)c3)nc(-c3ccc4ccc5ccc6ccc7nc(-c8ccccc8)oc7c6c5c4c3)n2)cc1]

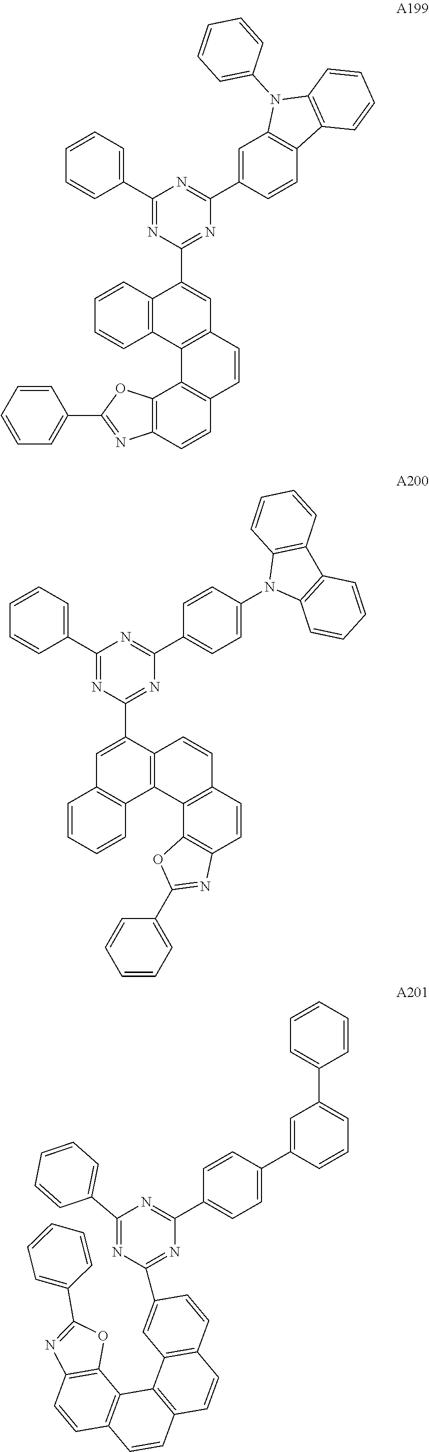 [SMILES: c1ccc(-c2nc(-c3ccc4c5ccccc5n(-c5ccccc5)c4c3)nc(-c3cc4ccc5ccc6nc(-c7ccccc7)oc6c5c4c4ccccc34)n2)cc1 ; c1ccc(-c2nc(-c3ccc(-n4c5ccccc5c5ccccc54)cc3)nc(-c3cc4ccccc4c4c3ccc3ccc5nc(-c6ccccc6)oc5c34)n2)cc1 ; c1ccc(-c2cccc(-c3ccc(-c4nc(-c5ccccc5)nc(-c5ccc6ccc7ccc8ccc9nc(-c%10ccccc%10)oc9c8c7c6c5)n4)cc3)c2)cc1]

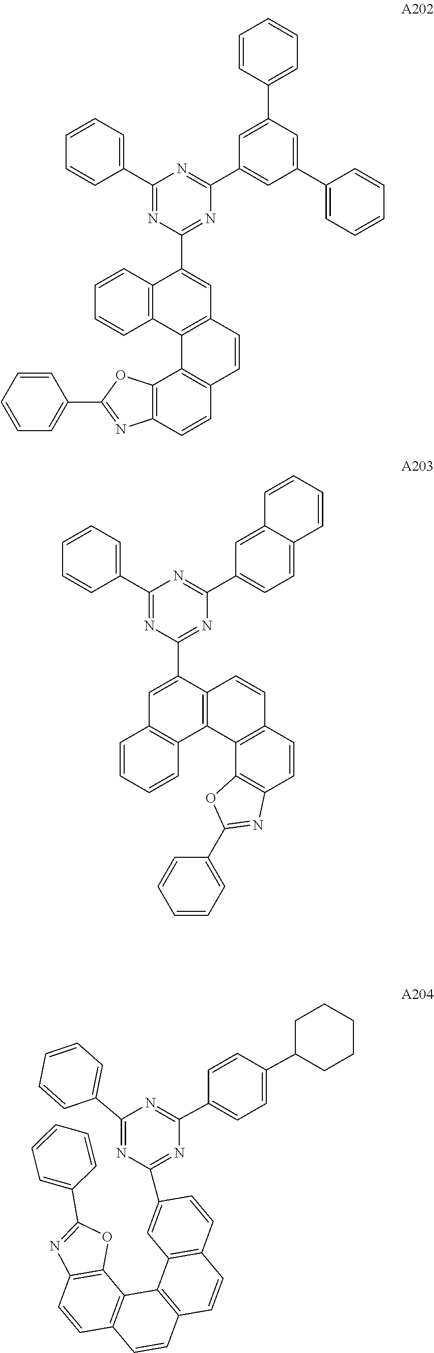 [SMILES: c1ccc(-c2cc(-c3ccccc3)cc(-c3nc(-c4ccccc4)nc(-c4cc5ccc6ccc7nc(-c8ccccc8)oc7c6c5c5ccccc45)n3)c2)cc1 ; c1ccc(-c2nc(-c3ccc4ccccc4c3)nc(-c3cc4ccccc4c4c3ccc3ccc5nc(-c6ccccc6)oc5c34)n2)cc1 ; c1ccc(-c2nc(-c3ccc(C4CCCCC4)cc3)nc(-c3ccc4ccc5ccc6ccc7nc(-c8ccccc8)oc7c6c5c4c3)n2)cc1]

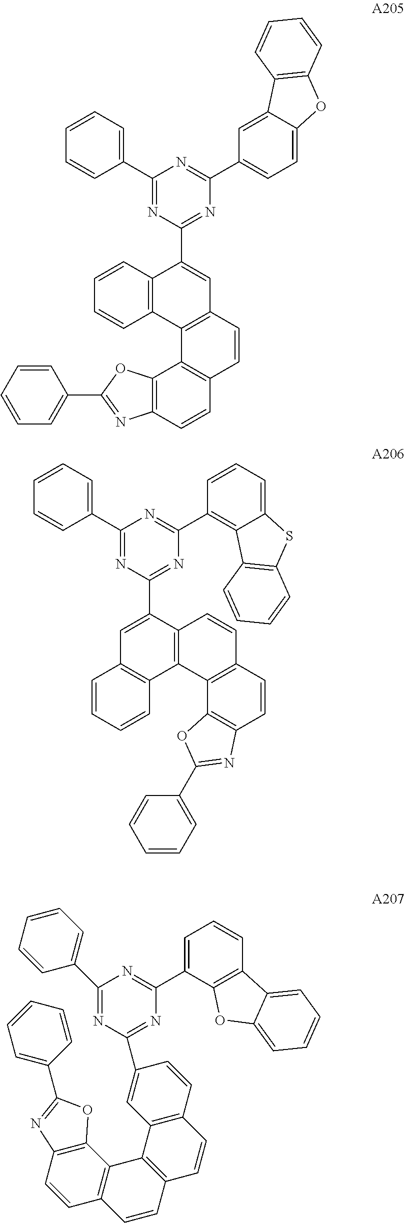

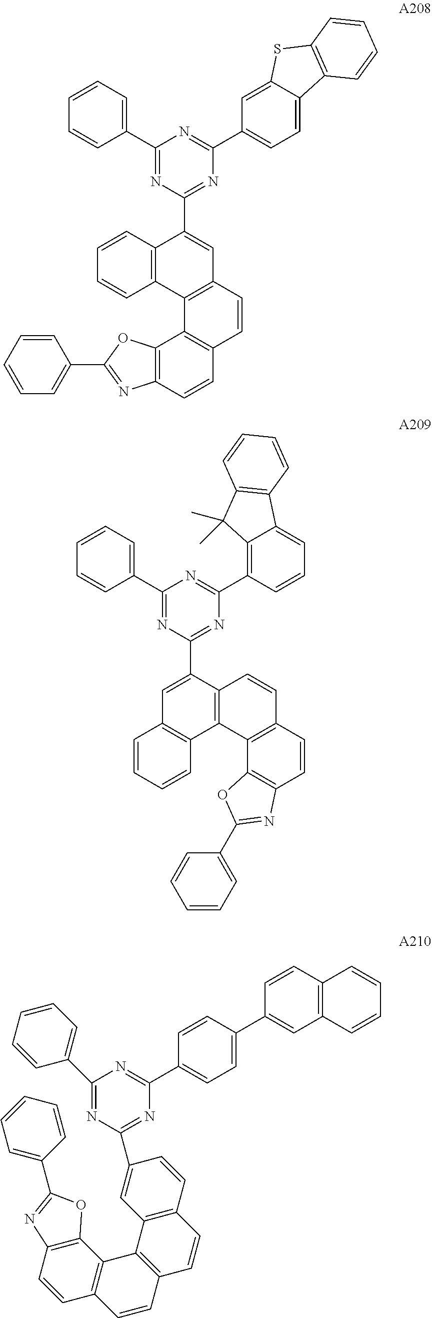 [SMILES: c1ccc(-c2nc(-c3ccc4c(c3)sc3ccccc34)nc(-c3cc4ccc5ccc6nc(-c7ccccc7)oc6c5c4c4ccccc34)n2)cc1 ; CC1(C)c2ccccc2-c2cccc(-c3nc(-c4ccccc4)nc(-c4cc5ccccc5c5c4ccc4ccc6nc(-c7ccccc7)oc6c45)n3)c21 ; c1ccc(-c2nc(-c3ccc(-c4ccc5ccccc5c4)cc3)nc(-c3ccc4ccc5ccc6ccc7nc(-c8ccccc8)oc7c6c5c4c3)n2)cc1]

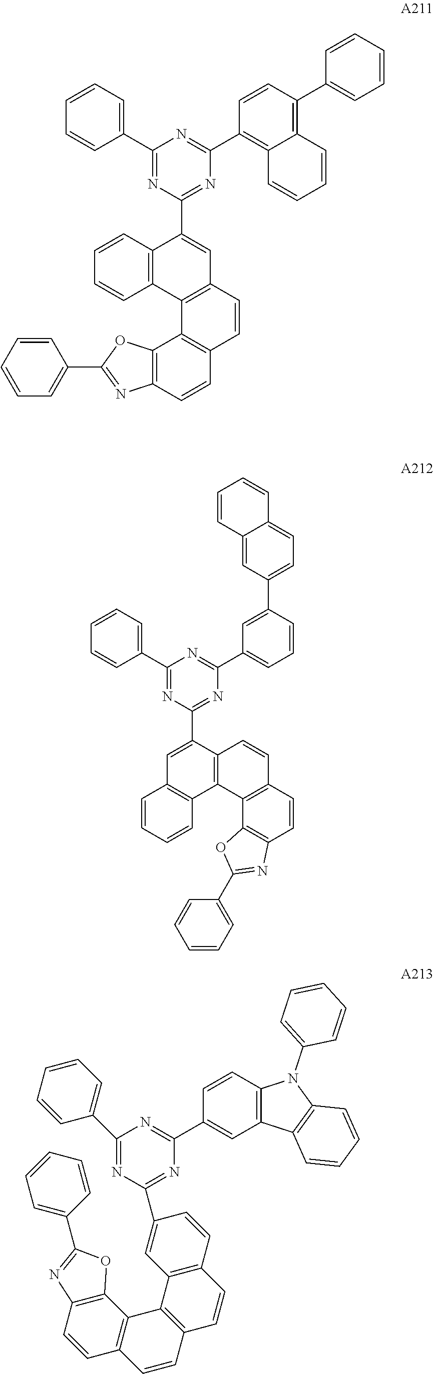 [SMILES: c1ccc(-c2nc(-c3ccc(-c4ccccc4)c4ccccc34)nc(-c3cc4ccc5ccc6nc(-c7ccccc7)oc6c5c4c4ccccc34)n2)cc1 ; c1ccc(-c2nc(-c3cccc(-c4ccc5ccccc5c4)c3)nc(-c3cc4ccccc4c4c3ccc3ccc5nc(-c6ccccc6)oc5c34)n2)cc1 ; c1ccc(-c2nc(-c3ccc4ccc5ccc6ccc7nc(-c8ccccc8)oc7c6c5c4c3)nc(-c3ccc4c(c3)c3ccccc3n4-c3ccccc3)n2)cc1]

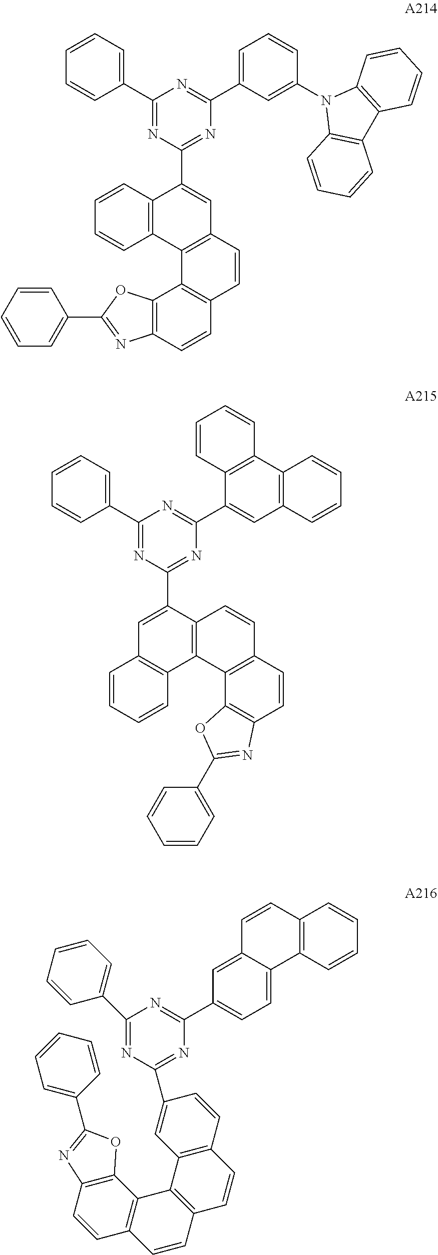 [SMILES: c1ccc(-c2nc(-c3cccc(-n4c5ccccc5c5ccccc54)c3)nc(-c3cc4ccc5ccc6nc(-c7ccccc7)oc6c5c4c4ccccc34)n2)cc1 ; c1ccc(-c2nc(-c3cc4ccccc4c4ccccc34)nc(-c3cc4ccccc4c4c3ccc3ccc5nc(-c6ccccc6)oc5c34)n2)cc1 ; c1ccc(-c2nc(-c3ccc4c(ccc5ccccc54)c3)nc(-c3ccc4ccc5ccc6ccc7nc(-c8ccccc8)oc7c6c5c4c3)n2)cc1]

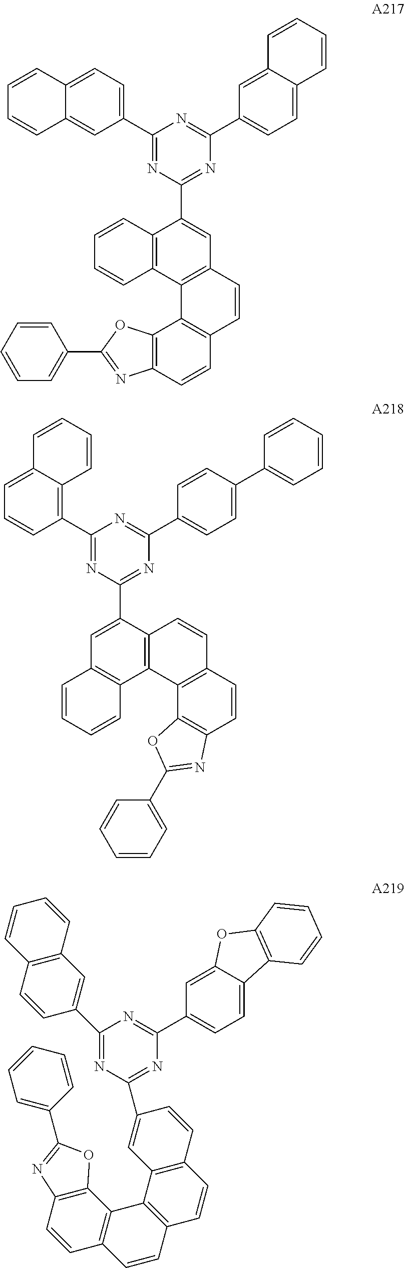

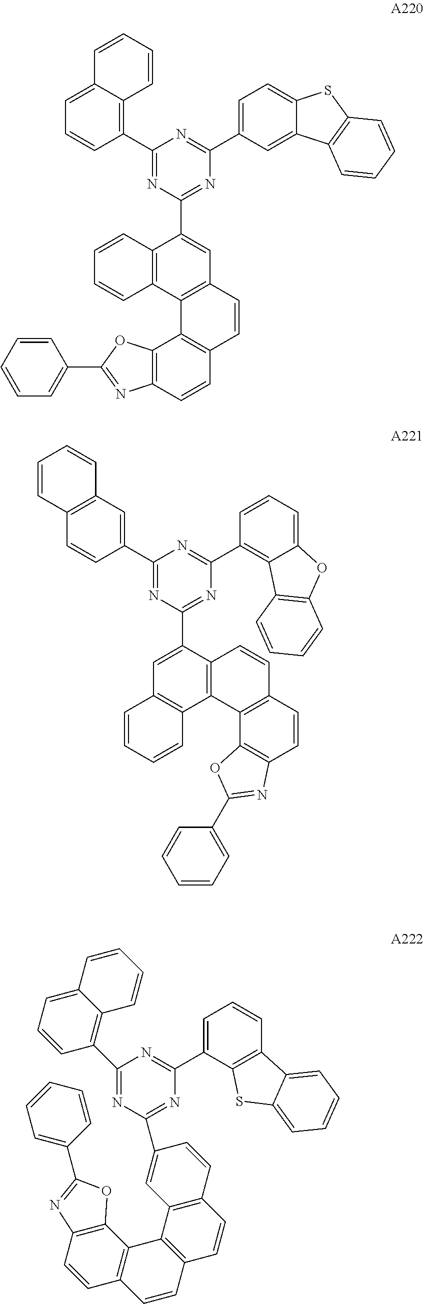 [SMILES: c1ccc(-c2nc3ccc4ccc5cc(-c6nc(-c7ccc8sc9ccccc9c8c7)nc(-c7cccc8ccccc78)n6)c6ccccc6c5c4c3o2)cc1 ; c1ccc(-c2nc3ccc4ccc5c(-c6nc(-c7ccc8ccccc8c7)nc(-c7cccc8oc9ccccc9c78)n6)cc6ccccc6c5c4c3o2)cc1 ; c1ccc(-c2nc3ccc4ccc5ccc6ccc(-c7nc(-c8cccc9ccccc89)nc(-c8cccc9c8sc8ccccc89)n7)cc6c5c4c3o2)cc1]

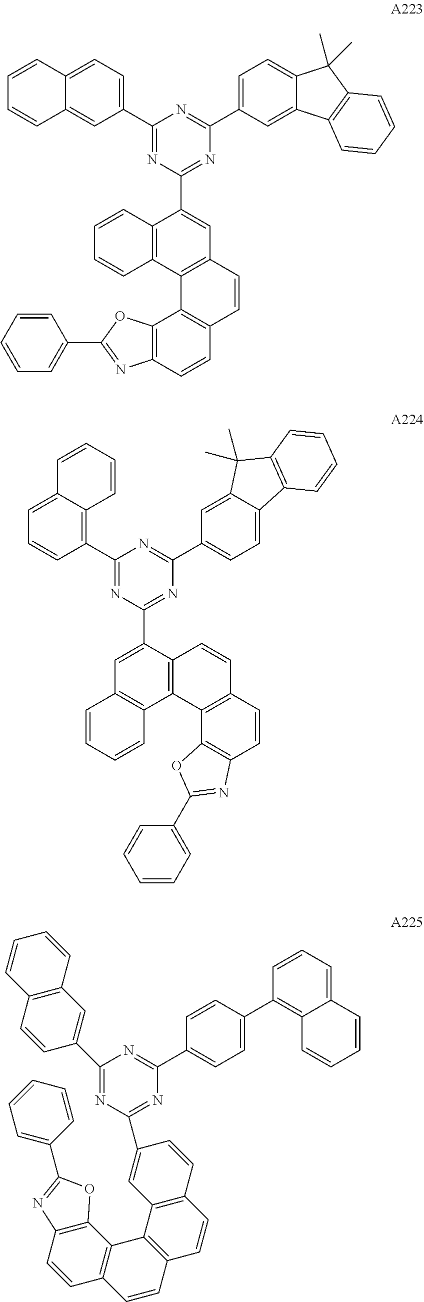 [SMILES: CC1(C)c2ccccc2-c2cc(-c3nc(-c4ccc5ccccc5c4)nc(-c4cc5ccc6ccc7nc(-c8ccccc8)oc7c6c5c5ccccc45)n3)ccc21 ; CC1(C)c2ccccc2-c2ccc(-c3nc(-c4cccc5ccccc45)nc(-c4cc5ccccc5c5c4ccc4ccc6nc(-c7ccccc7)oc6c45)n3)cc21 ; c1ccc(-c2nc3ccc4ccc5ccc6ccc(-c7nc(-c8ccc(-c9cccc%10ccccc9%10)cc8)nc(-c8ccc9ccccc9c8)n7)cc6c5c4c3o2)cc1]

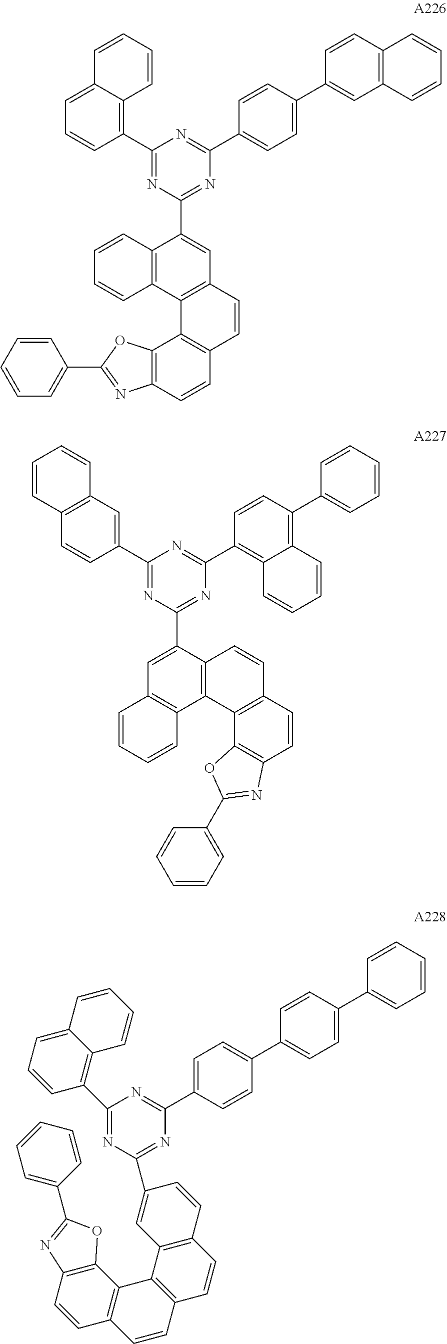 [SMILES: c1ccc(-c2nc3ccc4ccc5cc(-c6nc(-c7ccc(-c8ccc9ccccc9c8)cc7)nc(-c7cccc8ccccc78)n6)c6ccccc6c5c4c3o2)cc1 ; c1ccc(-c2nc3ccc4ccc5c(-c6nc(-c7ccc8ccccc8c7)nc(-c7ccc(-c8ccccc8)c8ccccc78)n6)cc6ccccc6c5c4c3o2)cc1 ; c1ccc(-c2ccc(-c3ccc(-c4nc(-c5ccc6ccc7ccc8ccc9nc(-c%10ccccc%10)oc9c8c7c6c5)nc(-c5cccc6ccccc56)n4)cc3)cc2)cc1]

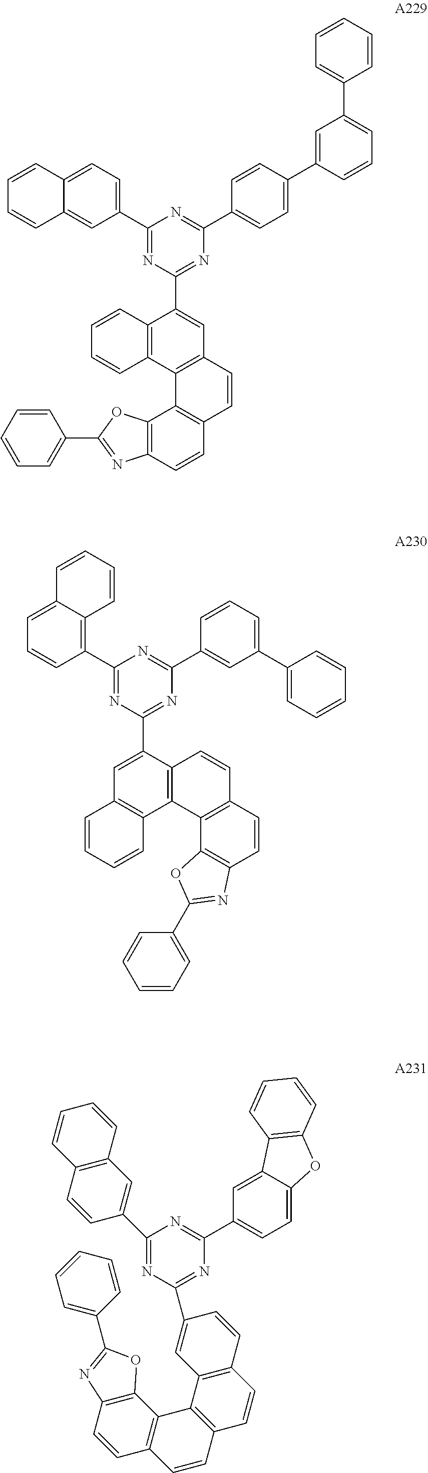 [SMILES: c1ccc(-c2cccc(-c3ccc(-c4nc(-c5ccc6ccccc6c5)nc(-c5cc6ccc7ccc8nc(-c9ccccc9)oc8c7c6c6ccccc56)n4)cc3)c2)cc1 ; c1ccc(-c2cccc(-c3nc(-c4cccc5ccccc45)nc(-c4cc5ccccc5c5c4ccc4ccc6nc(-c7ccccc7)oc6c45)n3)c2)cc1 ; c1ccc(-c2nc3ccc4ccc5ccc6ccc(-c7nc(-c8ccc9ccccc9c8)nc(-c8ccc9oc%10ccccc%10c9c8)n7)cc6c5c4c3o2)cc1]

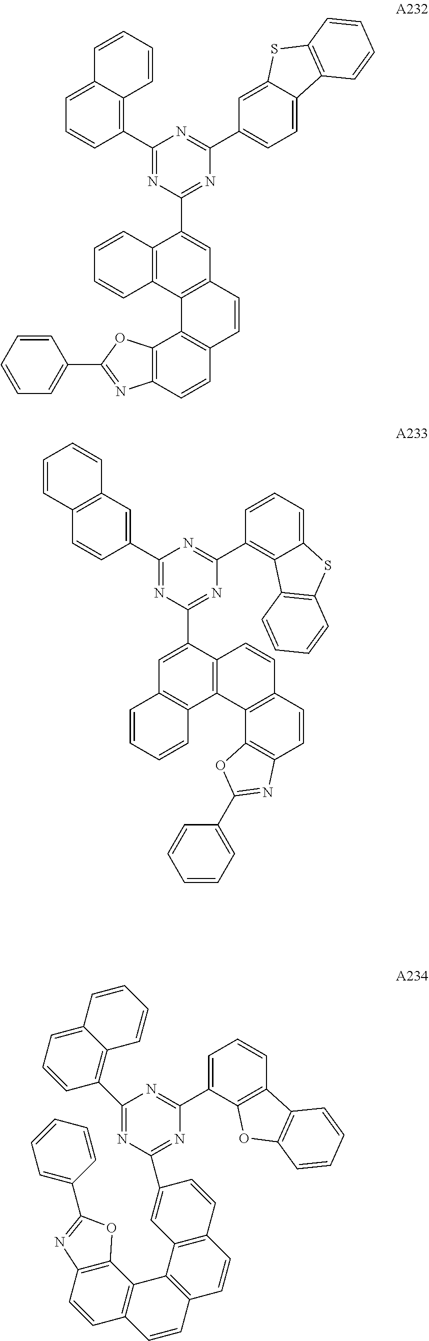 [SMILES: c1ccc(-c2nc3ccc4ccc5cc(-c6nc(-c7ccc8c(c7)sc7ccccc78)nc(-c7cccc8ccccc78)n6)c6ccccc6c5c4c3o2)cc1 ; c1ccc(-c2nc3ccc4ccc5c(-c6nc(-c7ccc8ccccc8c7)nc(-c7cccc8sc9ccccc9c78)n6)cc6ccccc6c5c4c3o2)cc1 ; c1ccc(-c2nc3ccc4ccc5ccc6ccc(-c7nc(-c8cccc9ccccc89)nc(-c8cccc9c8oc8ccccc89)n7)cc6c5c4c3o2)cc1]

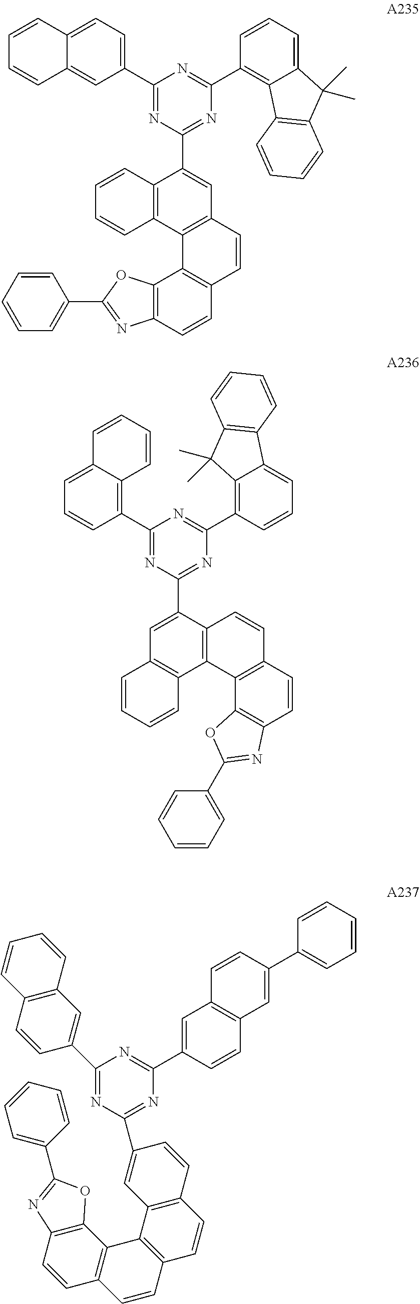 [SMILES: CC1(C)c2ccccc2-c2c(-c3nc(-c4ccc5ccccc5c4)nc(-c4cc5ccc6ccc7nc(-c8ccccc8)oc7c6c5c5ccccc45)n3)cccc21 ; CC1(C)c2ccccc2-c2cccc(-c3nc(-c4cccc5ccccc45)nc(-c4cc5ccccc5c5c4ccc4ccc6nc(-c7ccccc7)oc6c45)n3)c21 ; c1ccc(-c2ccc3cc(-c4nc(-c5ccc6ccccc6c5)nc(-c5ccc6ccc7ccc8ccc9nc(-c%10ccccc%10)oc9c8c7c6c5)n4)ccc3c2)cc1]

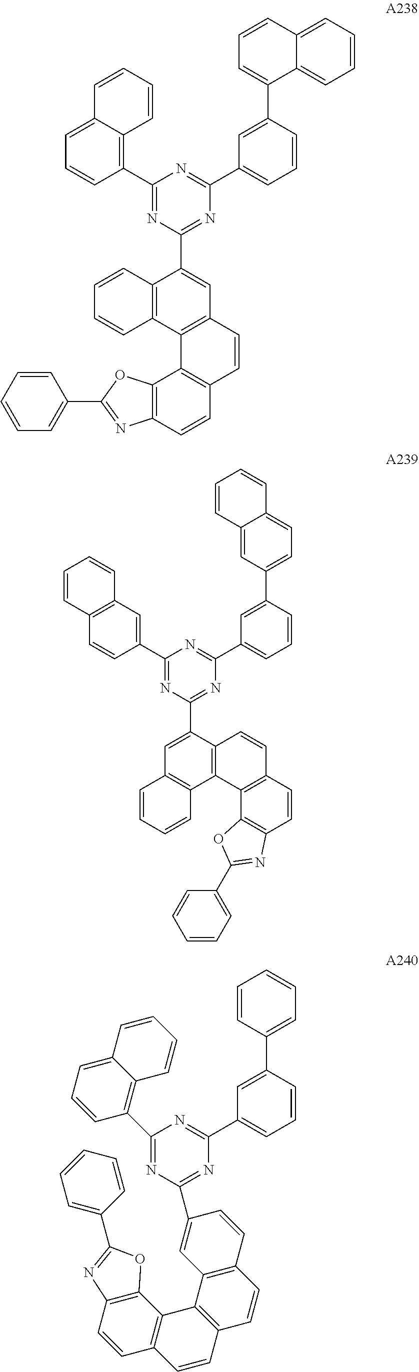 [SMILES: c1ccc(-c2nc3ccc4ccc5cc(-c6nc(-c7cccc(-c8cccc9ccccc89)c7)nc(-c7cccc8ccccc78)n6)c6ccccc6c5c4c3o2)cc1 ; c1ccc(-c2nc3ccc4ccc5c(-c6nc(-c7cccc(-c8ccc9ccccc9c8)c7)nc(-c7ccc8ccccc8c7)n6)cc6ccccc6c5c4c3o2)cc1 ; c1ccc(-c2cccc(-c3nc(-c4ccc5ccc6ccc7ccc8nc(-c9ccccc9)oc8c7c6c5c4)nc(-c4cccc5ccccc45)n3)c2)cc1]

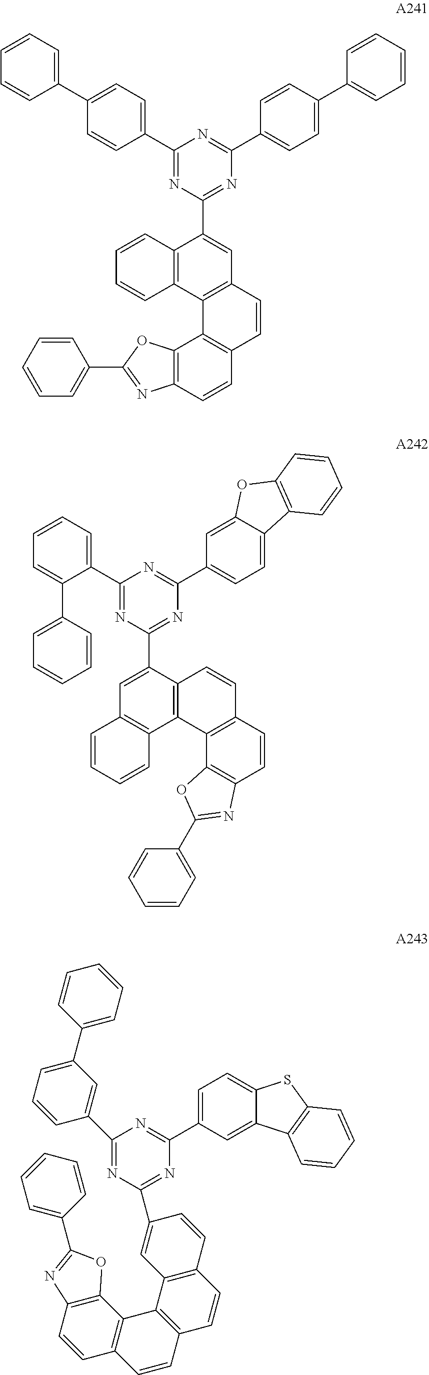 [SMILES: c1ccc(-c2ccc(-c3nc(-c4ccc(-c5ccccc5)cc4)nc(-c4cc5ccc6ccc7nc(-c8ccccc8)oc7c6c5c5ccccc45)n3)cc2)cc1 ; c1ccc(-c2nc3ccc4ccc5c(-c6nc(-c7ccc8c(c7)oc7ccccc78)nc(-c7ccccc7-c7ccccc7)n6)cc6ccccc6c5c4c3o2)cc1 ; c1ccc(-c2cccc(-c3nc(-c4ccc5sc6ccccc6c5c4)nc(-c4ccc5ccc6ccc7ccc8nc(-c9ccccc9)oc8c7c6c5c4)n3)c2)cc1]

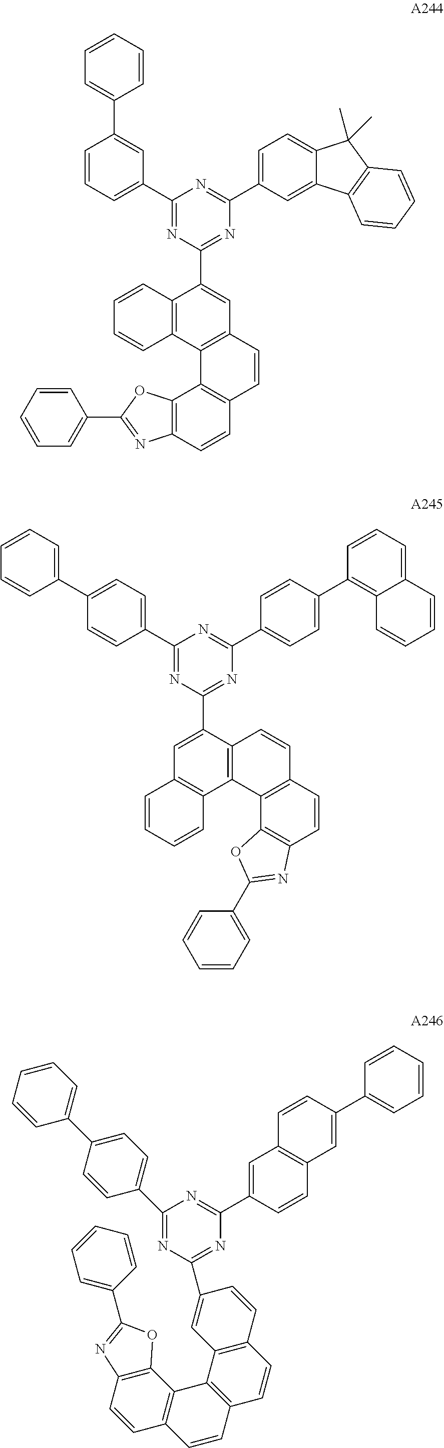 [SMILES: CC1(C)c2ccccc2-c2cc(-c3nc(-c4cccc(-c5ccccc5)c4)nc(-c4cc5ccc6ccc7nc(-c8ccccc8)oc7c6c5c5ccccc45)n3)ccc21 ; c1ccc(-c2ccc(-c3nc(-c4ccc(-c5cccc6ccccc56)cc4)nc(-c4cc5ccccc5c5c4ccc4ccc6nc(-c7ccccc7)oc6c45)n3)cc2)cc1 ; c1ccc(-c2ccc(-c3nc(-c4ccc5cc(-c6ccccc6)ccc5c4)nc(-c4ccc5ccc6ccc7ccc8nc(-c9ccccc9)oc8c7c6c5c4)n3)cc2)cc1]

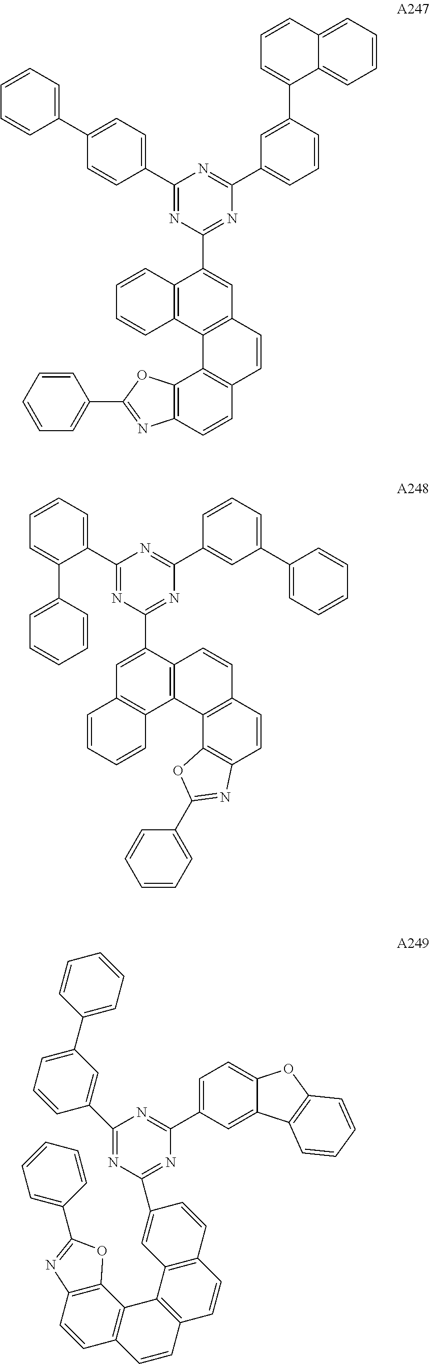 [SMILES: c1ccc(-c2ccc(-c3nc(-c4cccc(-c5cccc6ccccc56)c4)nc(-c4cc5ccc6ccc7nc(-c8ccccc8)oc7c6c5c5ccccc45)n3)cc2)cc1 ; c1ccc(-c2cccc(-c3nc(-c4ccccc4-c4ccccc4)nc(-c4cc5ccccc5c5c4ccc4ccc6nc(-c7ccccc7)oc6c45)n3)c2)cc1 ; c1ccc(-c2cccc(-c3nc(-c4ccc5oc6ccccc6c5c4)nc(-c4ccc5ccc6ccc7ccc8nc(-c9ccccc9)oc8c7c6c5c4)n3)c2)cc1]

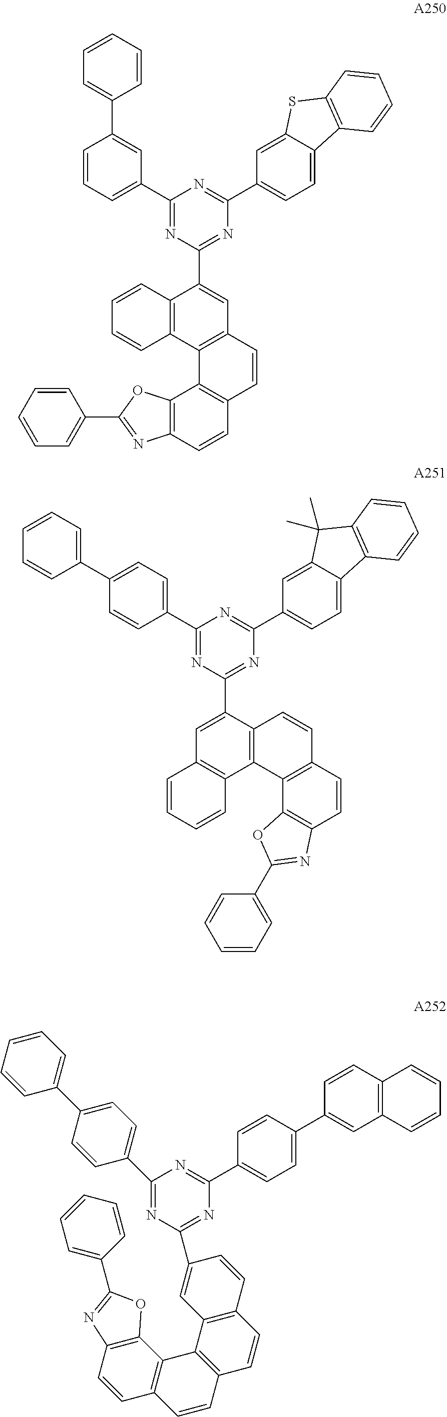 [SMILES: c1ccc(-c2cccc(-c3nc(-c4ccc5c(c4)sc4ccccc45)nc(-c4cc5ccc6ccc7nc(-c8ccccc8)oc7c6c5c5ccccc45)n3)c2)cc1 ; CC1(C)c2ccccc2-c2ccc(-c3nc(-c4ccc(-c5ccccc5)cc4)nc(-c4cc5ccccc5c5c4ccc4ccc6nc(-c7ccccc7)oc6c45)n3)cc21 ; c1ccc(-c2ccc(-c3nc(-c4ccc(-c5ccc6ccccc6c5)cc4)nc(-c4ccc5ccc6ccc7ccc8nc(-c9ccccc9)oc8c7c6c5c4)n3)cc2)cc1]

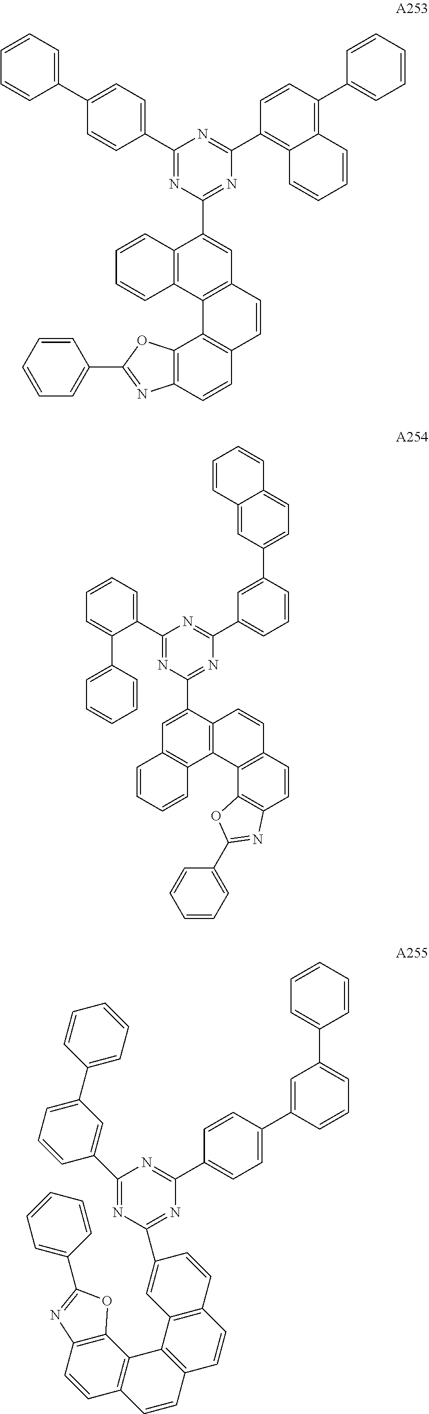 [SMILES: c1ccc(-c2ccc(-c3nc(-c4ccc(-c5ccccc5)c5ccccc45)nc(-c4cc5ccc6ccc7nc(-c8ccccc8)oc7c6c5c5ccccc45)n3)cc2)cc1 ; c1ccc(-c2nc3ccc4ccc5c(-c6nc(-c7cccc(-c8ccc9ccccc9c8)c7)nc(-c7ccccc7-c7ccccc7)n6)cc6ccccc6c5c4c3o2)cc1 ; c1ccc(-c2cccc(-c3ccc(-c4nc(-c5cccc(-c6ccccc6)c5)nc(-c5ccc6ccc7ccc8ccc9nc(-c%10ccccc%10)oc9c8c7c6c5)n4)cc3)c2)cc1]

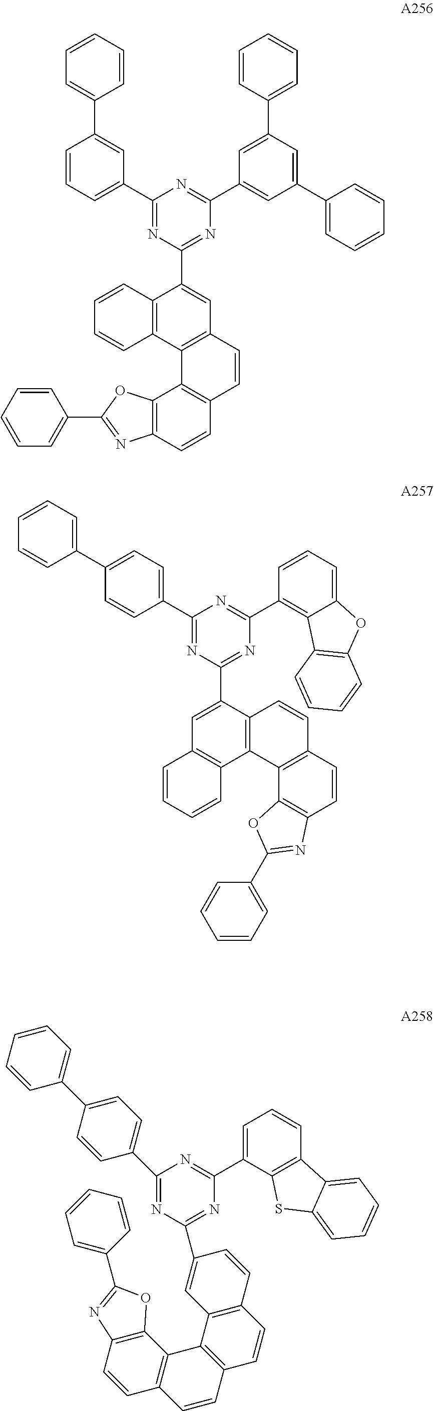 [SMILES: c1ccc(-c2cccc(-c3nc(-c4cc(-c5ccccc5)cc(-c5ccccc5)c4)nc(-c4cc5ccc6ccc7nc(-c8ccccc8)oc7c6c5c5ccccc45)n3)c2)cc1 ; c1ccc(-c2ccc(-c3nc(-c4cc5ccccc5c5c4ccc4ccc6nc(-c7ccccc7)oc6c45)nc(-c4cccc5oc6ccccc6c45)n3)cc2)cc1 ; c1ccc(-c2ccc(-c3nc(-c4ccc5ccc6ccc7ccc8nc(-c9ccccc9)oc8c7c6c5c4)nc(-c4cccc5c4sc4ccccc45)n3)cc2)cc1]

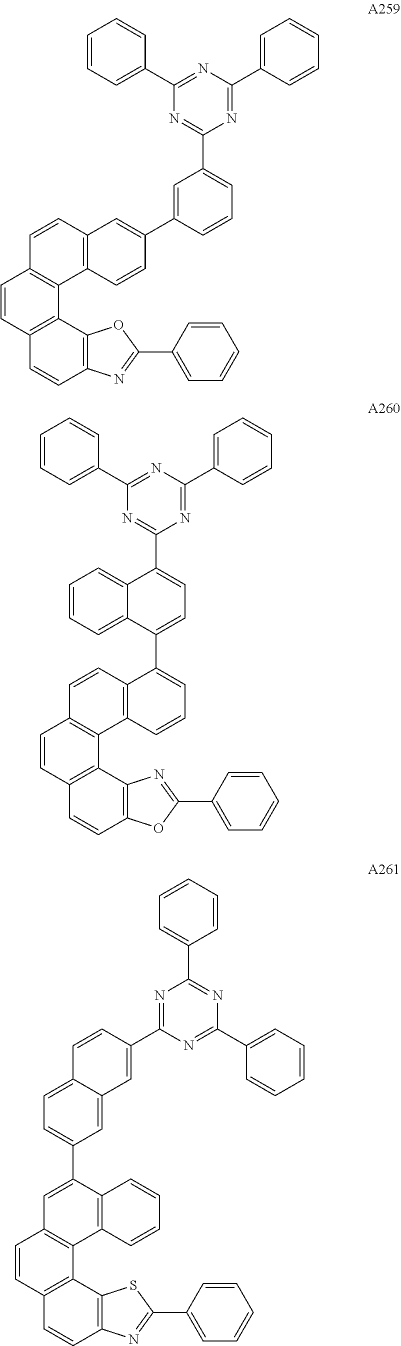

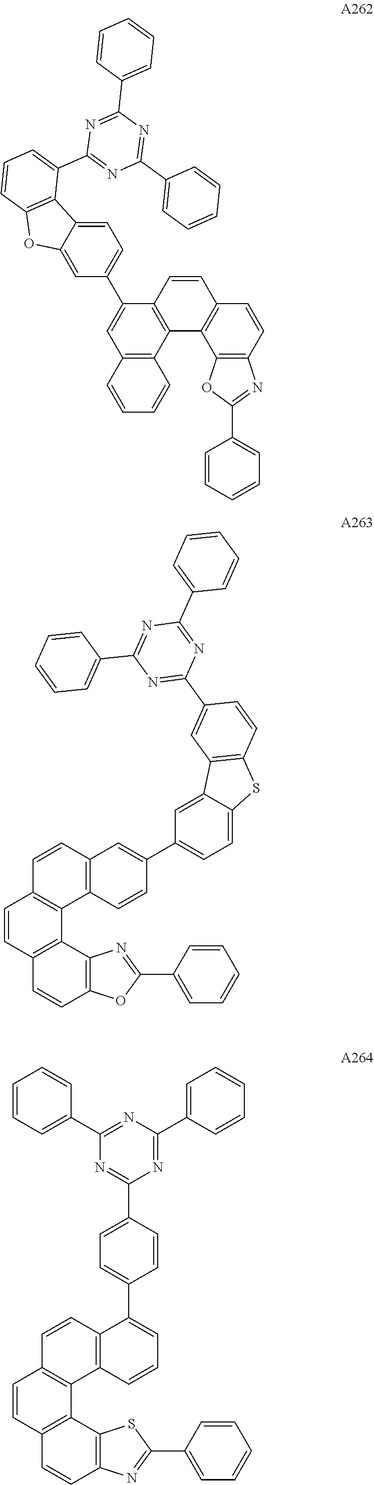

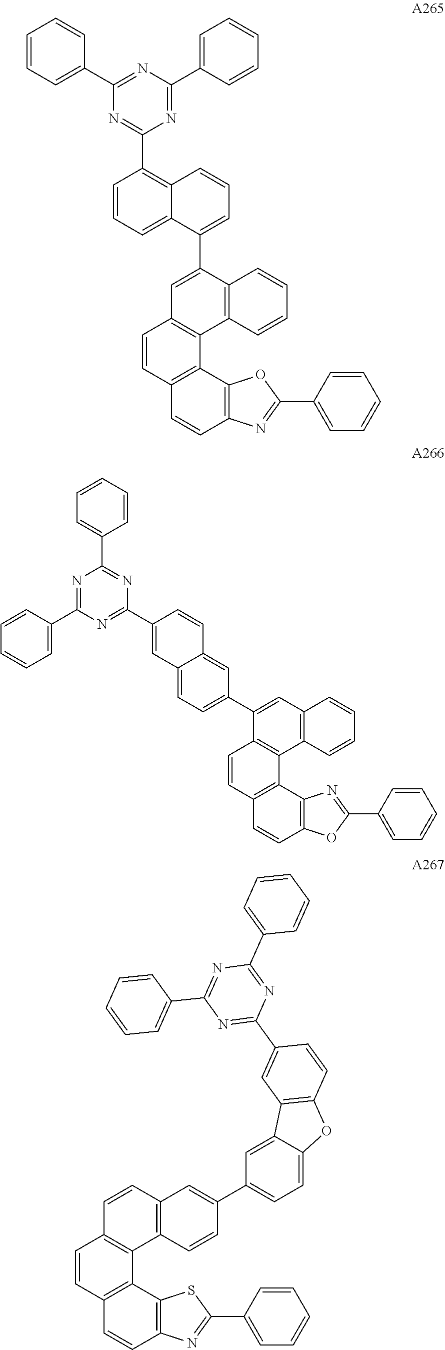

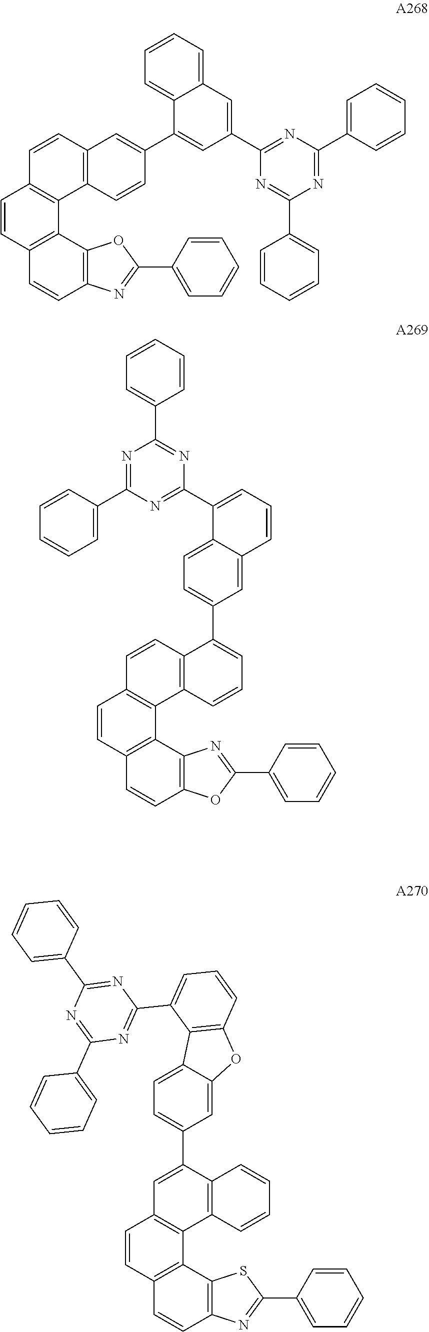 [SMILES: c1ccc(-c2nc(-c3ccccc3)nc(-c3cc(-c4ccc5c(ccc6ccc7ccc8nc(-c9ccccc9)oc8c7c65)c4)c4ccccc4c3)n2)cc1 ; c1ccc(-c2nc(-c3ccccc3)nc(-c3cccc4cc(-c5cccc6c5ccc5ccc7ccc8oc(-c9ccccc9)nc8c7c56)ccc34)n2)cc1 ; c1ccc(-c2nc(-c3ccccc3)nc(-c3cccc4oc5cc(-c6cc7ccc8ccc9nc(-c%10ccccc%10)sc9c8c7c7ccccc67)ccc5c34)n2)cc1]

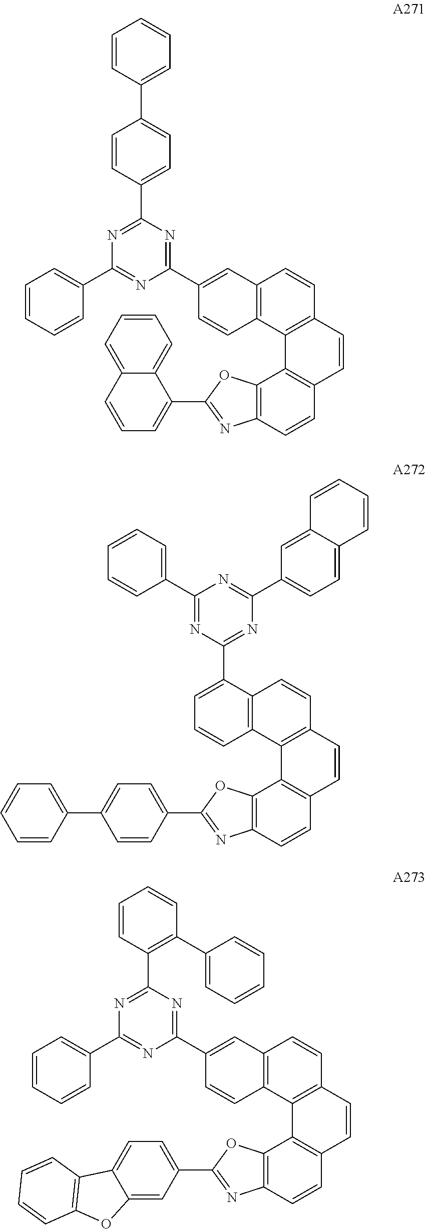

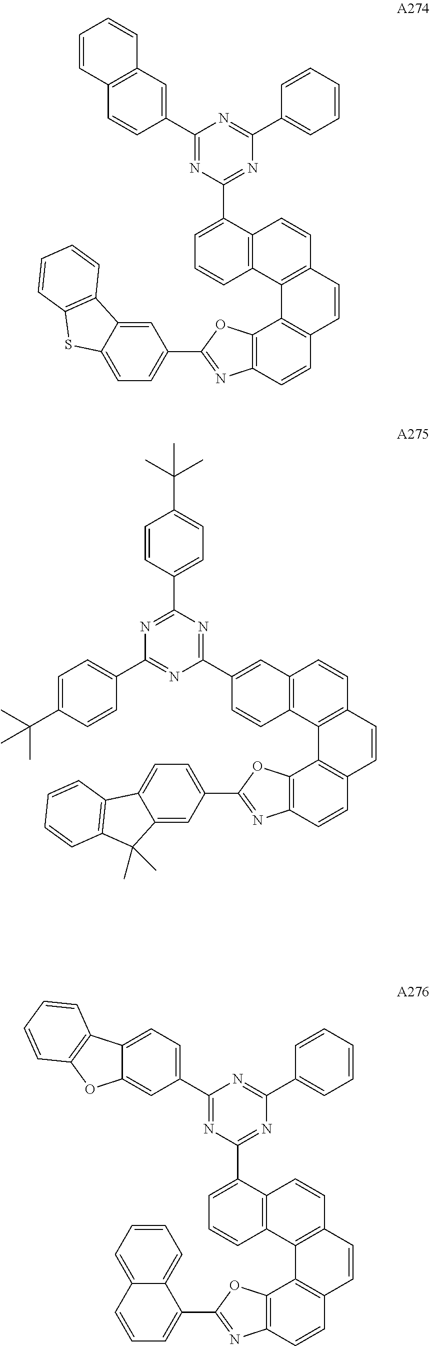 [SMILES: c1ccc(-c2nc(-c3ccc4ccccc4c3)nc(-c3cccc4c3ccc3ccc5ccc6nc(-c7ccc8sc9ccccc9c8c7)oc6c5c34)n2)cc1 ; CC(C)(C)c1ccc(-c2nc(-c3ccc(C(C)(C)C)cc3)nc(-c3ccc4c(ccc5ccc6ccc7nc(-c8ccc9c(c8)C(C)(C)c8ccccc8-9)oc7c6c54)c3)n2)cc1 ; c1ccc(-c2nc(-c3ccc4c(c3)oc3ccccc34)nc(-c3cccc4c3ccc3ccc5ccc6nc(-c7cccc8ccccc78)oc6c5c34)n2)cc1]

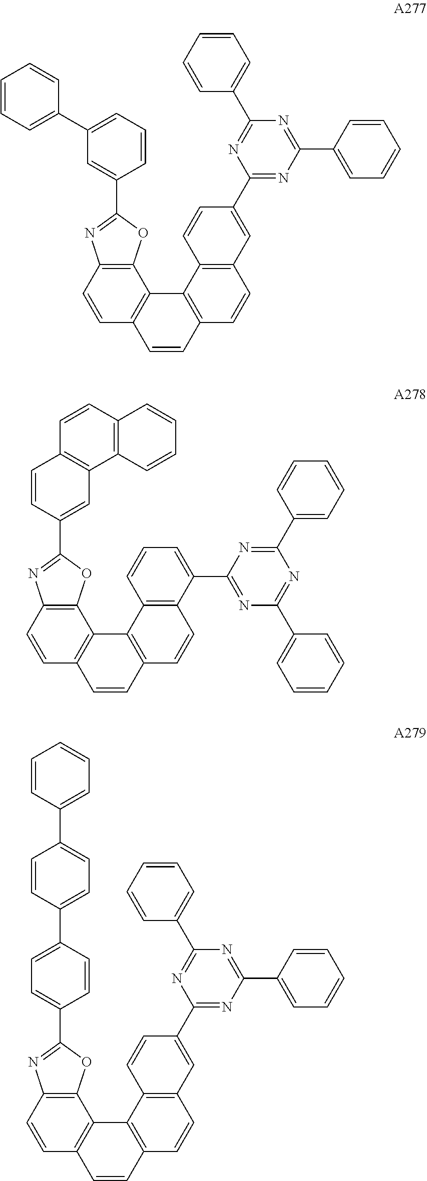 [SMILES: c1ccc(-c2cccc(-c3nc4ccc5ccc6ccc7cc(-c8nc(-c9ccccc9)nc(-c9ccccc9)n8)ccc7c6c5c4o3)c2)cc1 ; c1ccc(-c2nc(-c3ccccc3)nc(-c3cccc4c3ccc3ccc5ccc6nc(-c7ccc8ccc9ccccc9c8c7)oc6c5c34)n2)cc1 ; c1ccc(-c2ccc(-c3ccc(-c4nc5ccc6ccc7ccc8cc(-c9nc(-c%10ccccc%10)nc(-c%10ccccc%10)n9)ccc8c7c6c5o4)cc3)cc2)cc1]

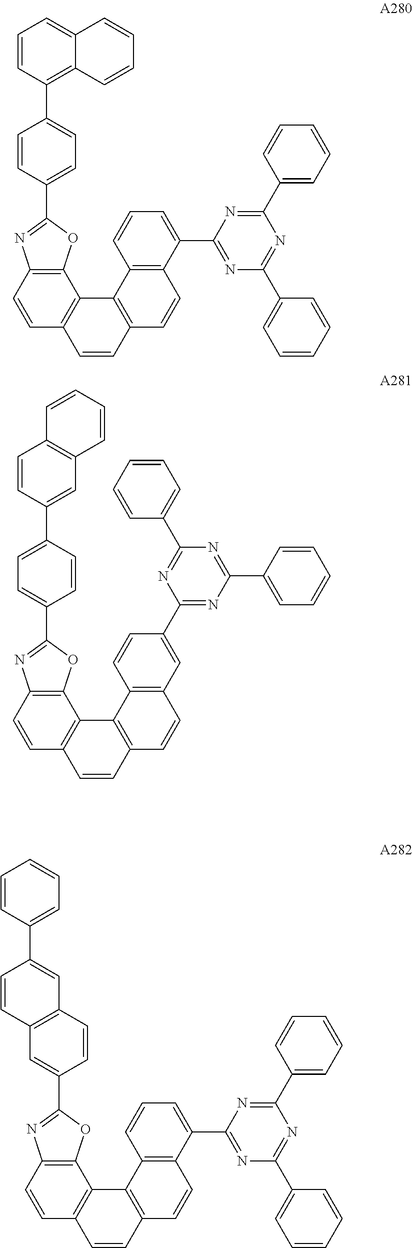

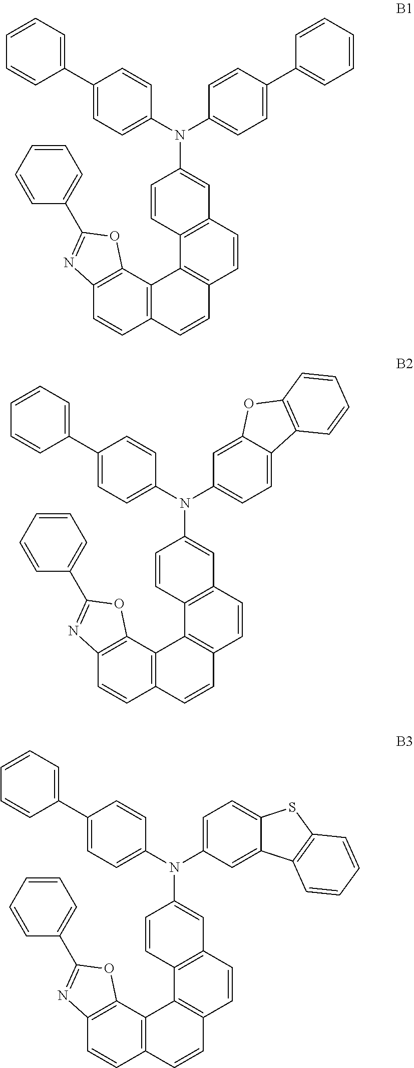 [SMILES: c1ccc(-c2ccc(N(c3ccc(-c4ccccc4)cc3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc4c(c3)oc3ccccc34)cc2)cc1 ; c1ccc(-c2ccc(N(c3ccc4c(ccc5ccc6ccc7nc(-c8ccccc8)oc7c6c54)c3)c3ccc4sc5ccccc5c4c3)cc2)cc1]

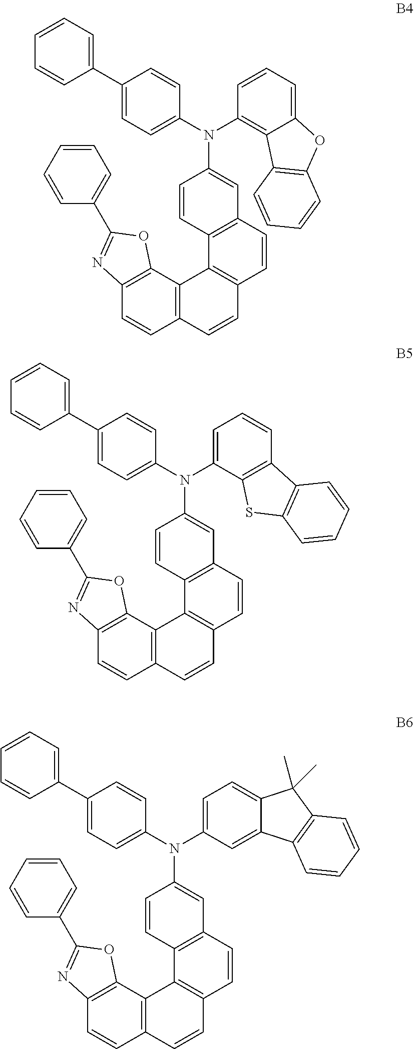 [SMILES: c1ccc(-c2ccc(N(c3ccc4c(ccc5ccc6ccc7nc(-c8ccccc8)oc7c6c54)c3)c3cccc4oc5ccccc5c34)cc2)cc1 ; c1ccc(-c2ccc(N(c3ccc4c(ccc5ccc6ccc7nc(-c8ccccc8)oc7c6c54)c3)c3cccc4c3sc3ccccc34)cc2)cc1 ; CC1(C)c2ccccc2-c2cc(N(c3ccc(-c4ccccc4)cc3)c3ccc4c(ccc5ccc6ccc7nc(-c8ccccc8)oc7c6c54)c3)ccc21]

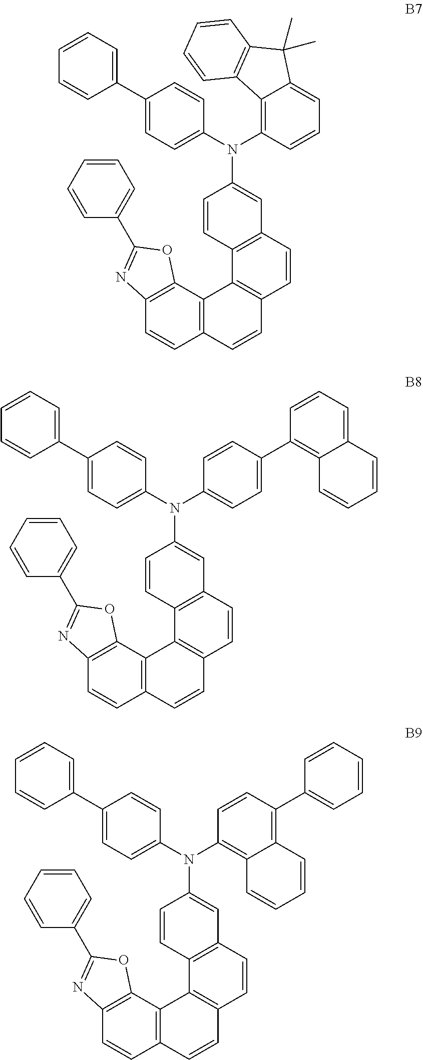 [SMILES: CC1(C)c2ccccc2-c2c(N(c3ccc(-c4ccccc4)cc3)c3ccc4c(ccc5ccc6ccc7nc(-c8ccccc8)oc7c6c54)c3)cccc21 ; c1ccc(-c2ccc(N(c3ccc(-c4cccc5ccccc45)cc3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc(-c4ccccc4)c4ccccc34)cc2)cc1]

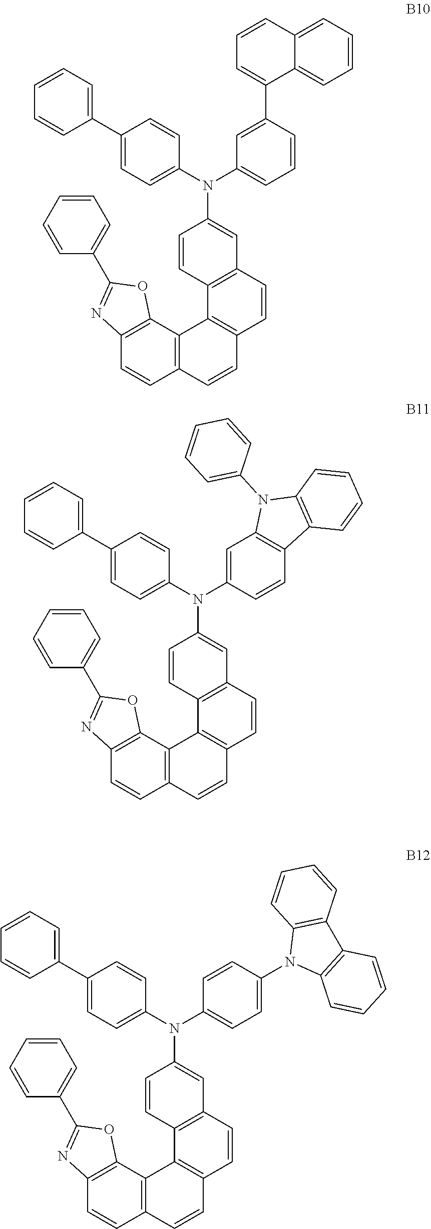

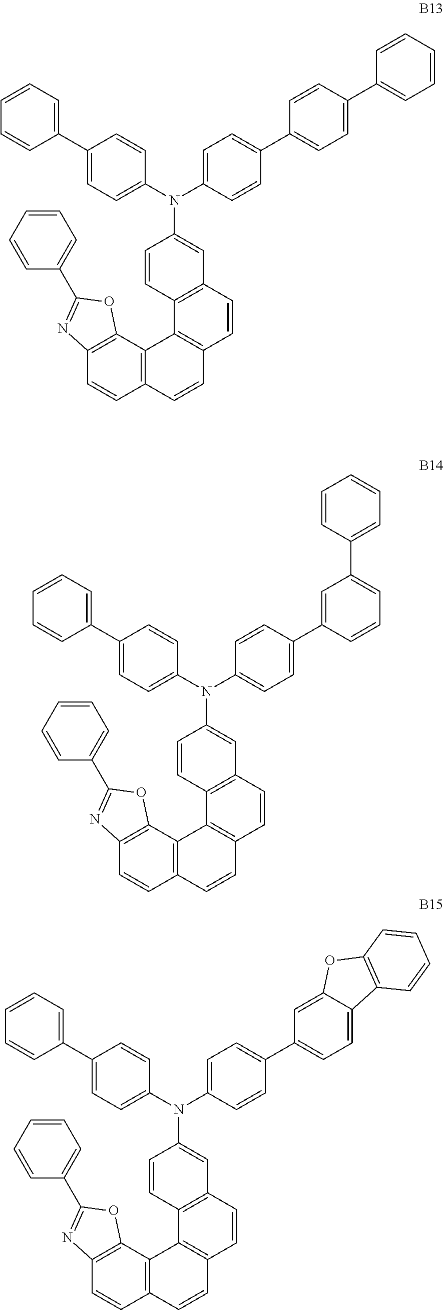 [SMILES: c1ccc(-c2ccc(-c3ccc(N(c4ccc(-c5ccccc5)cc4)c4ccc5c(ccc6ccc7ccc8nc(-c9ccccc9)oc8c7c65)c4)cc3)cc2)cc1 ; c1ccc(-c2ccc(N(c3ccc(-c4cccc(-c5ccccc5)c4)cc3)c3ccc4c(ccc5ccc6ccc7nc(-c8ccccc8)oc7c6c54)c3)cc2)cc1 ; c1ccc(-c2ccc(N(c3ccc(-c4ccc5c(c4)oc4ccccc45)cc3)c3ccc4c(ccc5ccc6ccc7nc(-c8ccccc8)oc7c6c54)c3)cc2)cc1]

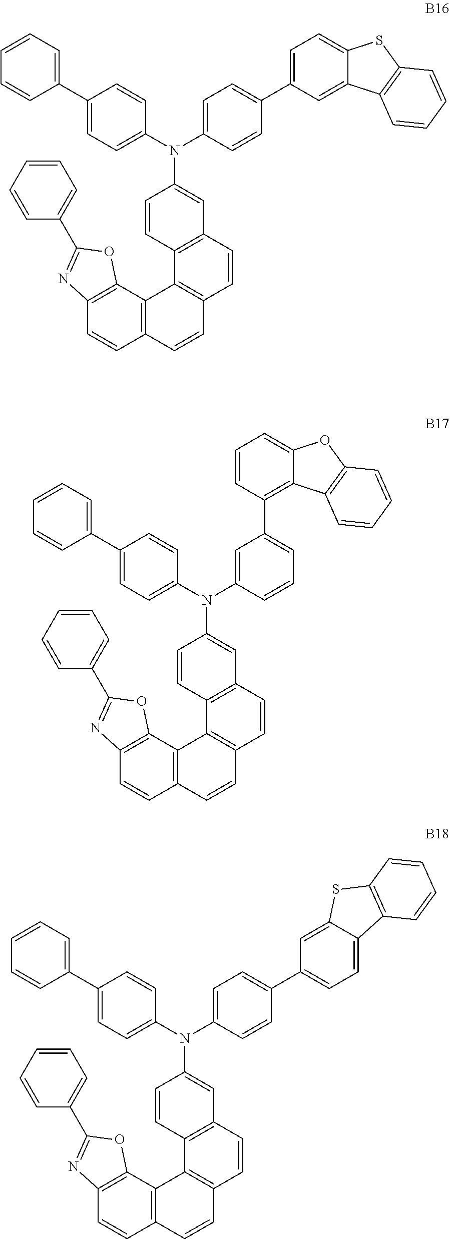 [SMILES: c1ccc(-c2ccc(N(c3ccc(-c4ccc5sc6ccccc6c5c4)cc3)c3ccc4c(ccc5ccc6ccc7nc(-c8ccccc8)oc7c6c54)c3)cc2)cc1 ; c1ccc(-c2ccc(N(c3cccc(-c4cccc5oc6ccccc6c45)c3)c3ccc4c(ccc5ccc6ccc7nc(-c8ccccc8)oc7c6c54)c3)cc2)cc1 ; c1ccc(-c2ccc(N(c3ccc(-c4ccc5c(c4)sc4ccccc45)cc3)c3ccc4c(ccc5ccc6ccc7nc(-c8ccccc8)oc7c6c54)c3)cc2)cc1]

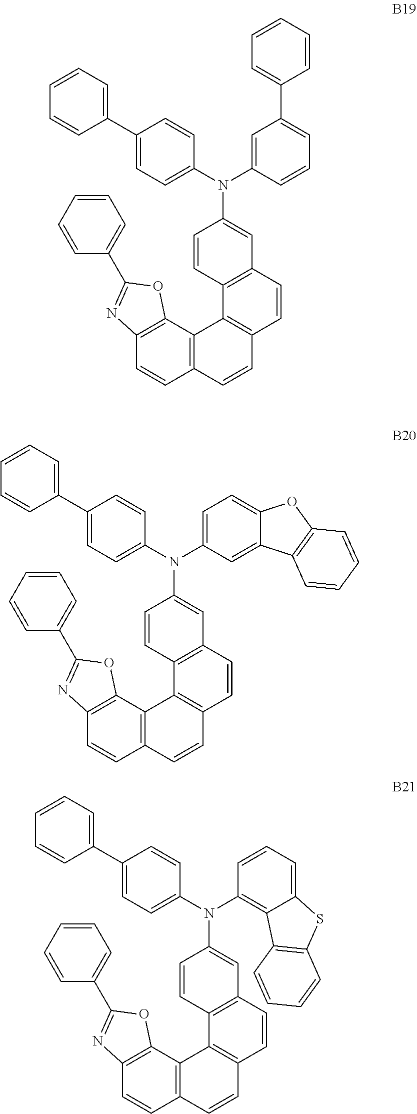 [SMILES: c1ccc(-c2ccc(N(c3cccc(-c4ccccc4)c3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc4oc5ccccc5c4c3)cc2)cc1 ; c1ccc(-c2ccc(N(c3ccc4c(ccc5ccc6ccc7nc(-c8ccccc8)oc7c6c54)c3)c3cccc4sc5ccccc5c34)cc2)cc1]

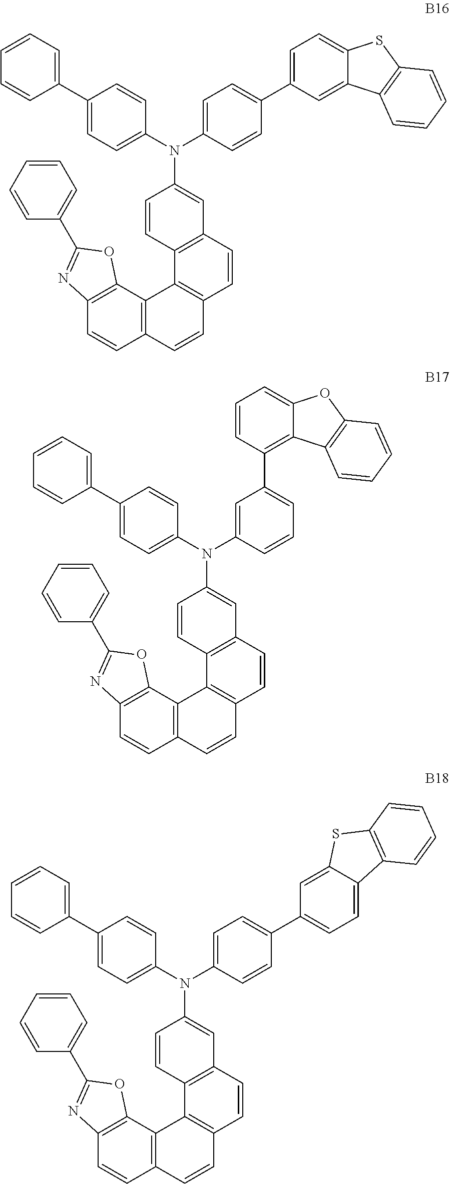 [SMILES: c1ccc(-c2ccc(N(c3ccc(-c4ccc5sc6ccccc6c5c4)cc3)c3ccc4c(ccc5ccc6ccc7nc(-c8ccccc8)oc7c6c54)c3)cc2)cc1 ; c1ccc(-c2ccc(N(c3cccc(-c4cccc5oc6ccccc6c45)c3)c3ccc4c(ccc5ccc6ccc7nc(-c8ccccc8)oc7c6c54)c3)cc2)cc1 ; c1ccc(-c2ccc(N(c3ccc(-c4ccc5c(c4)sc4ccccc45)cc3)c3ccc4c(ccc5ccc6ccc7nc(-c8ccccc8)oc7c6c54)c3)cc2)cc1]

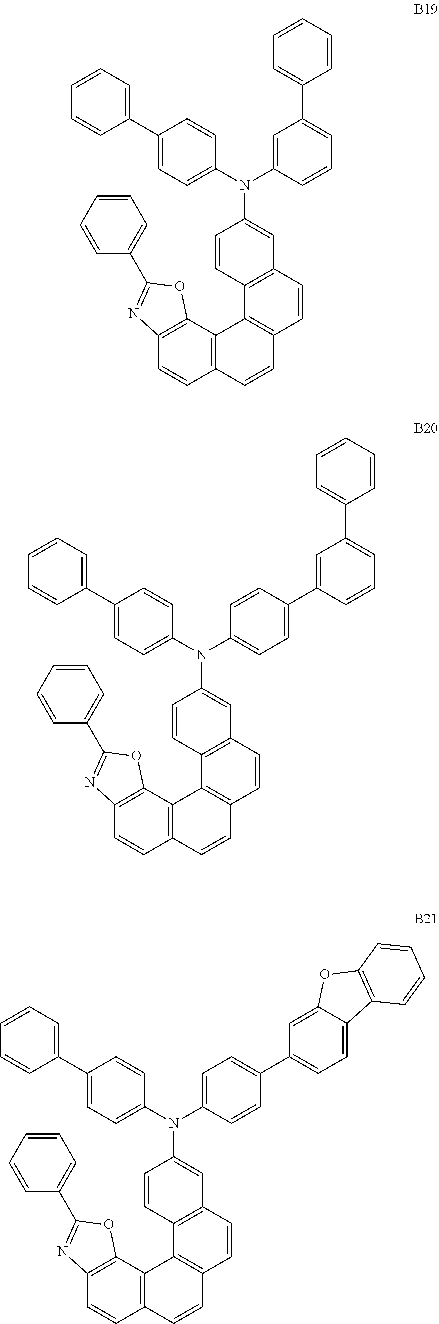 [SMILES: c1ccc(-c2ccc(N(c3cccc(-c4ccccc4)c3)c3ccc4c(ccc5ccc6ccc7nc(-c8ccccc8)oc7c6c54)c3)cc2)cc1 ; c1ccc(-c2ccc(N(c3ccc(-c4cccc(-c5ccccc5)c4)cc3)c3ccc4c(ccc5ccc6ccc7nc(-c8ccccc8)oc7c6c54)c3)cc2)cc1 ; c1ccc(-c2ccc(N(c3ccc(-c4ccc5c(c4)oc4ccccc45)cc3)c3ccc4c(ccc5ccc6ccc7nc(-c8ccccc8)oc7c6c54)c3)cc2)cc1]

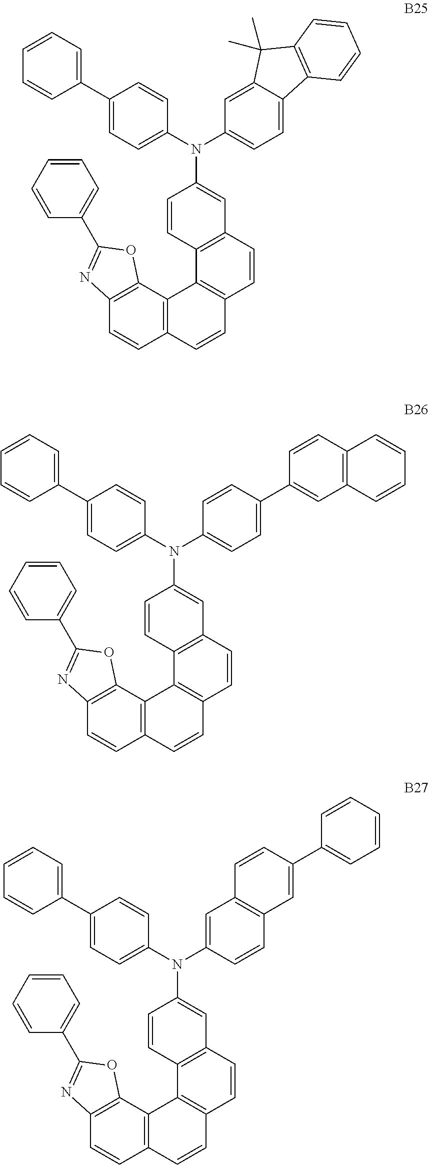 [SMILES: CC1(C)c2ccccc2-c2ccc(N(c3ccc(-c4ccccc4)cc3)c3ccc4c(ccc5ccc6ccc7nc(-c8ccccc8)oc7c6c54)c3)cc21 ; c1ccc(-c2ccc(N(c3ccc(-c4ccc5ccccc5c4)cc3)c3ccc4c(ccc5ccc6ccc7nc(-c8ccccc8)oc7c6c54)c3)cc2)cc1 ; c1ccc(-c2ccc(N(c3ccc4cc(-c5ccccc5)ccc4c3)c3ccc4c(ccc5ccc6ccc7nc(-c8ccccc8)oc7c6c54)c3)cc2)cc1]

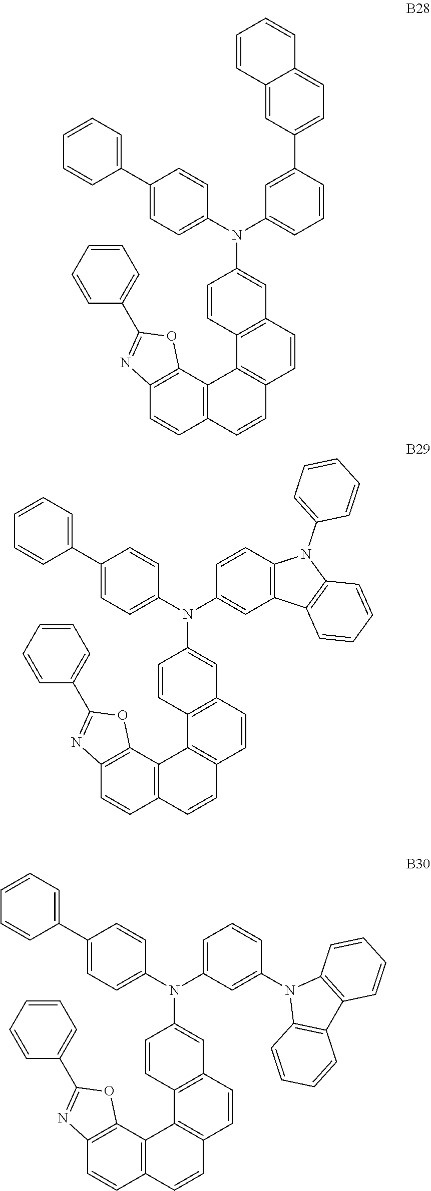 [SMILES: c1ccc(-c2ccc(N(c3cccc(-c4ccc5ccccc5c4)c3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc4c(c3)c3ccccc3n4-c3ccccc3)cc2)cc1 ; c1ccc(-c2ccc(N(c3cccc(-n4c5ccccc5c5ccccc54)c3)c3ccc4c(ccc5ccc6ccc7nc(-c8ccccc8)oc7c6c54)c3)cc2)cc1]

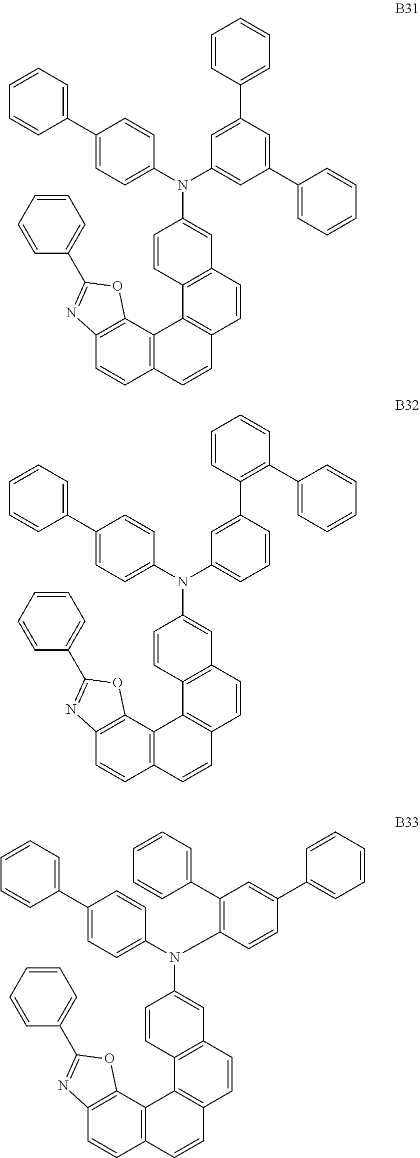 [SMILES: c1ccc(-c2ccc(N(c3cc(-c4ccccc4)cc(-c4ccccc4)c3)c3ccc4c(ccc5ccc6ccc7nc(-c8ccccc8)oc7c6c54)c3)cc2)cc1 ; c1ccc(-c2ccc(N(c3cccc(-c4ccccc4-c4ccccc4)c3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc(-c4ccccc4)cc3-c3ccccc3)cc2)cc1]

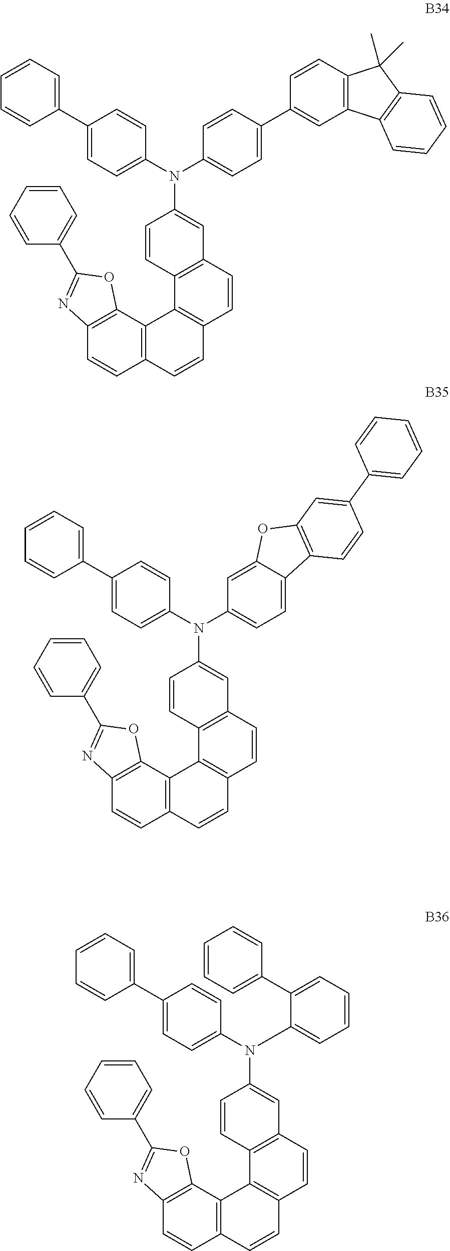 [SMILES: CC1(C)c2ccccc2-c2cc(-c3ccc(N(c4ccc(-c5ccccc5)cc4)c4ccc5c(ccc6ccc7ccc8nc(-c9ccccc9)oc8c7c65)c4)cc3)ccc21 ; c1ccc(-c2ccc(N(c3ccc4c(ccc5ccc6ccc7nc(-c8ccccc8)oc7c6c54)c3)c3ccc4c(c3)oc3cc(-c5ccccc5)ccc34)cc2)cc1 ; c1ccc(-c2ccc(N(c3ccc4c(ccc5ccc6ccc7nc(-c8ccccc8)oc7c6c54)c3)c3ccccc3-c3ccccc3)cc2)cc1]

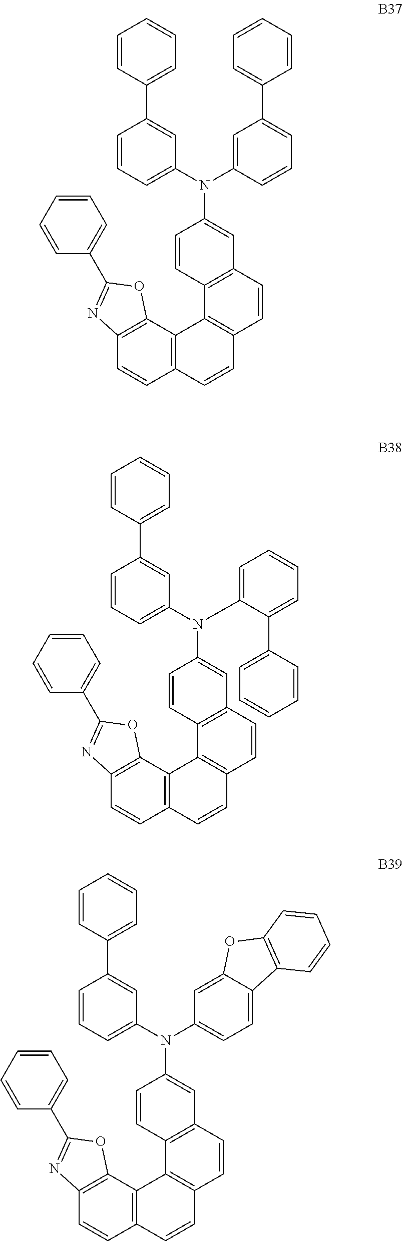 [SMILES: c1ccc(-c2cccc(N(c3cccc(-c4ccccc4)c3)c3ccc4c(ccc5ccc6ccc7nc(-c8ccccc8)oc7c6c54)c3)c2)cc1 ; c1ccc(-c2cccc(N(c3ccc4c(ccc5ccc6ccc7nc(-c8ccccc8)oc7c6c54)c3)c3ccccc3-c3ccccc3)c2)cc1 ; c1ccc(-c2cccc(N(c3ccc4c(ccc5ccc6ccc7nc(-c8ccccc8)oc7c6c54)c3)c3ccc4c(c3)oc3ccccc34)c2)cc1]

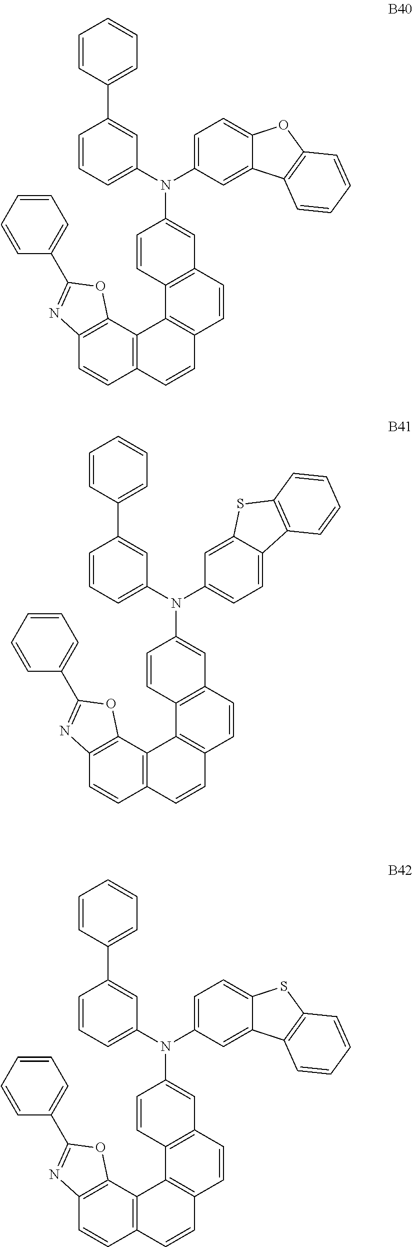 [SMILES: c1ccc(-c2cccc(N(c3ccc4c(ccc5ccc6ccc7nc(-c8ccccc8)oc7c6c54)c3)c3ccc4oc5ccccc5c4c3)c2)cc1 ; c1ccc(-c2cccc(N(c3ccc4c(ccc5ccc6ccc7nc(-c8ccccc8)oc7c6c54)c3)c3ccc4c(c3)sc3ccccc34)c2)cc1 ; c1ccc(-c2cccc(N(c3ccc4c(ccc5ccc6ccc7nc(-c8ccccc8)oc7c6c54)c3)c3ccc4sc5ccccc5c4c3)c2)cc1]

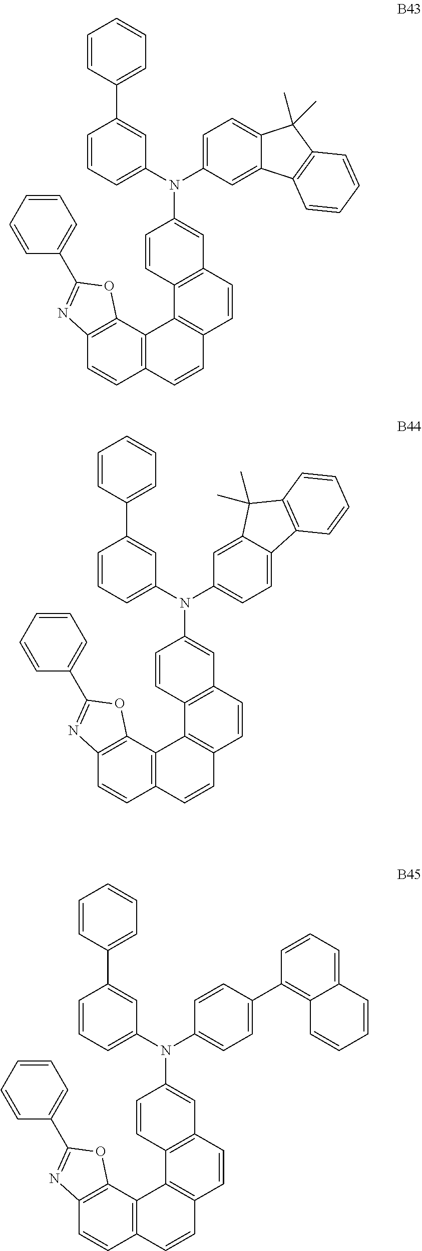 [SMILES: CC1(C)c2ccccc2-c2cc(N(c3cccc(-c4ccccc4)c3)c3ccc4c(ccc5ccc6ccc7nc(-c8ccccc8)oc7c6c54)c3)ccc21 ; CC1(C)c2ccccc2-c2ccc(N(c3cccc(-c4ccccc4)c3)c3ccc4c(ccc5ccc6ccc7nc(-c8ccccc8)oc7c6c54)c3)cc21 ; c1ccc(-c2cccc(N(c3ccc(-c4cccc5ccccc45)cc3)c3ccc4c(ccc5ccc6ccc7nc(-c8ccccc8)oc7c6c54)c3)c2)cc1]

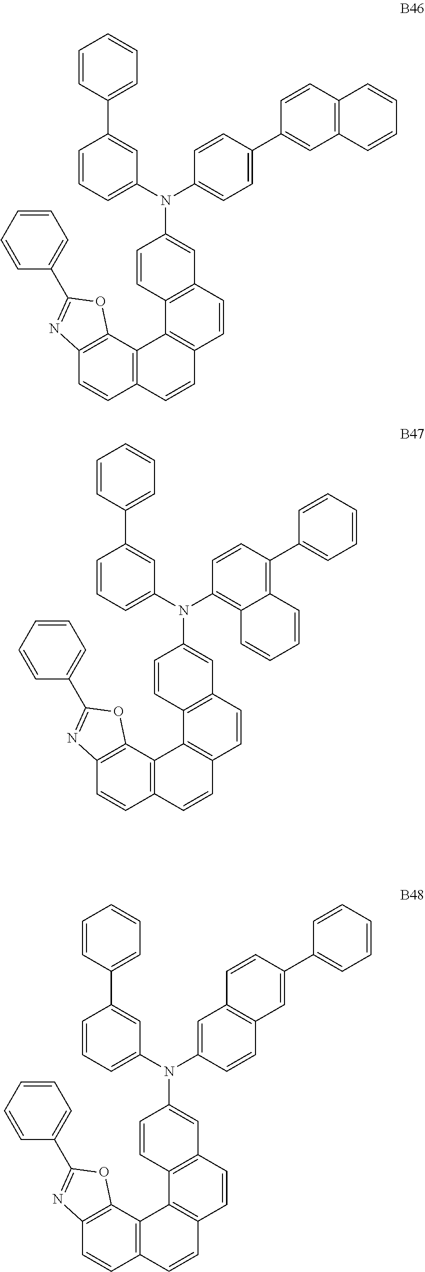 [SMILES: c1ccc(-c2cccc(N(c3ccc(-c4ccc5ccccc5c4)cc3)c3ccc4c(ccc5ccc6ccc7nc(-c8ccccc8)oc7c6c54)c3)c2)cc1 ; c1ccc(-c2cccc(N(c3ccc4c(ccc5ccc6ccc7nc(-c8ccccc8)oc7c6c54)c3)c3ccc(-c4ccccc4)c4ccccc34)c2)cc1 ; c1ccc(-c2cccc(N(c3ccc4cc(-c5ccccc5)ccc4c3)c3ccc4c(ccc5ccc6ccc7nc(-c8ccccc8)oc7c6c54)c3)c2)cc1]

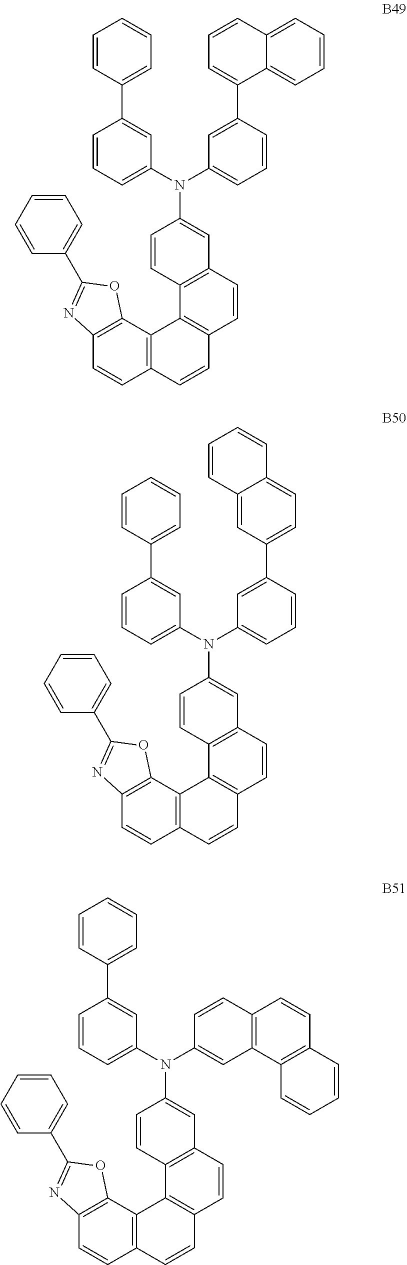 [SMILES: c1ccc(-c2cccc(N(c3cccc(-c4cccc5ccccc45)c3)c3ccc4c(ccc5ccc6ccc7nc(-c8ccccc8)oc7c6c54)c3)c2)cc1 ; c1ccc(-c2cccc(N(c3cccc(-c4ccc5ccccc5c4)c3)c3ccc4c(ccc5ccc6ccc7nc(-c8ccccc8)oc7c6c54)c3)c2)cc1 ; c1ccc(-c2cccc(N(c3ccc4c(ccc5ccc6ccc7nc(-c8ccccc8)oc7c6c54)c3)c3ccc4ccc5ccccc5c4c3)c2)cc1]

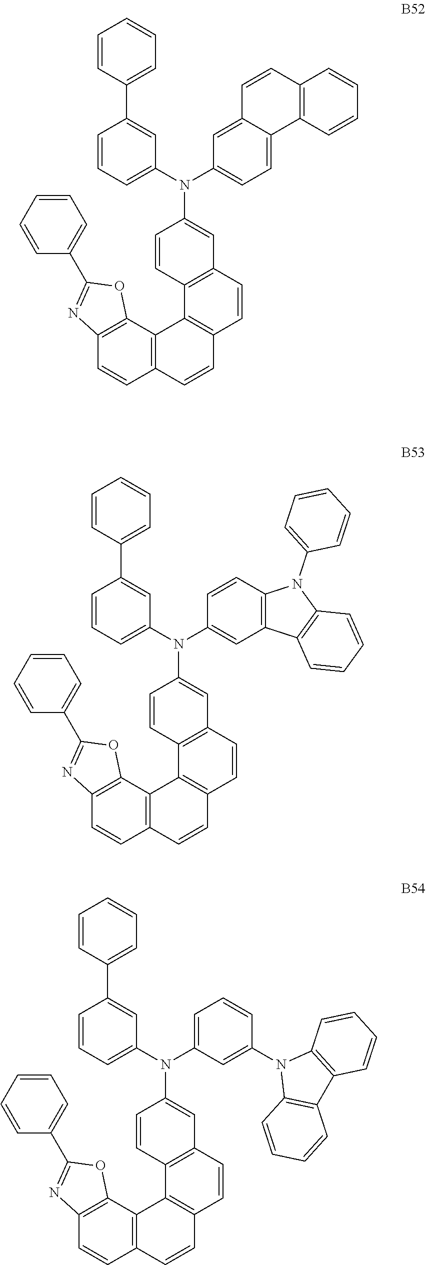 [SMILES: c1ccc(-c2cccc(N(c3ccc4c(ccc5ccccc54)c3)c3ccc4c(ccc5ccc6ccc7nc(-c8ccccc8)oc7c6c54)c3)c2)cc1 ; c1ccc(-c2cccc(N(c3ccc4c(ccc5ccc6ccc7nc(-c8ccccc8)oc7c6c54)c3)c3ccc4c(c3)c3ccccc3n4-c3ccccc3)c2)cc1 ; c1ccc(-c2cccc(N(c3cccc(-n4c5ccccc5c5ccccc54)c3)c3ccc4c(ccc5ccc6ccc7nc(-c8ccccc8)oc7c6c54)c3)c2)cc1]

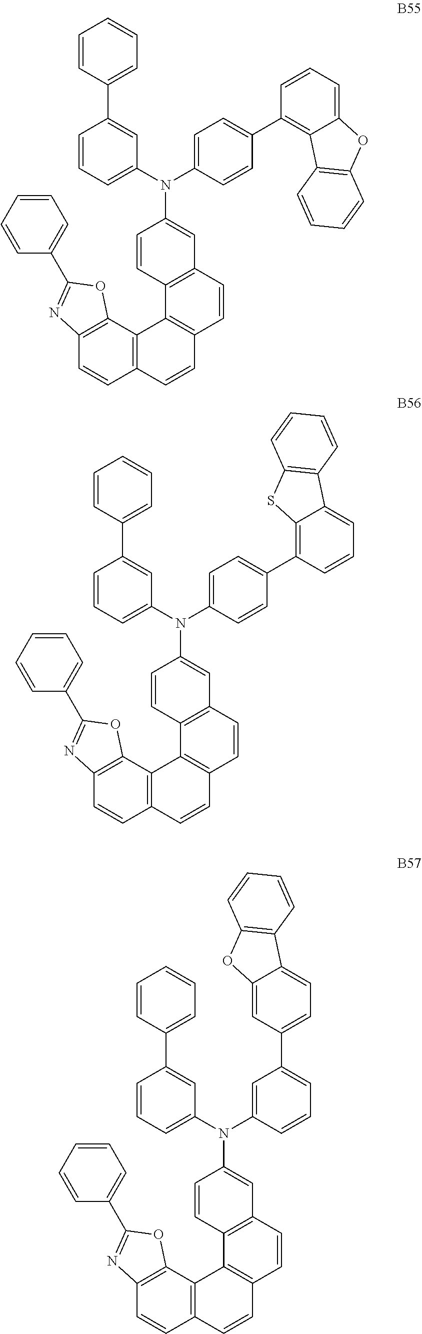 [SMILES: c1ccc(-c2cccc(N(c3ccc(-c4cccc5oc6ccccc6c45)cc3)c3ccc4c(ccc5ccc6ccc7nc(-c8ccccc8)oc7c6c54)c3)c2)cc1 ; c1ccc(-c2cccc(N(c3ccc(-c4cccc5c4sc4ccccc45)cc3)c3ccc4c(ccc5ccc6ccc7nc(-c8ccccc8)oc7c6c54)c3)c2)cc1 ; c1ccc(-c2cccc(N(c3cccc(-c4ccc5c(c4)oc4ccccc45)c3)c3ccc4c(ccc5ccc6ccc7nc(-c8ccccc8)oc7c6c54)c3)c2)cc1]

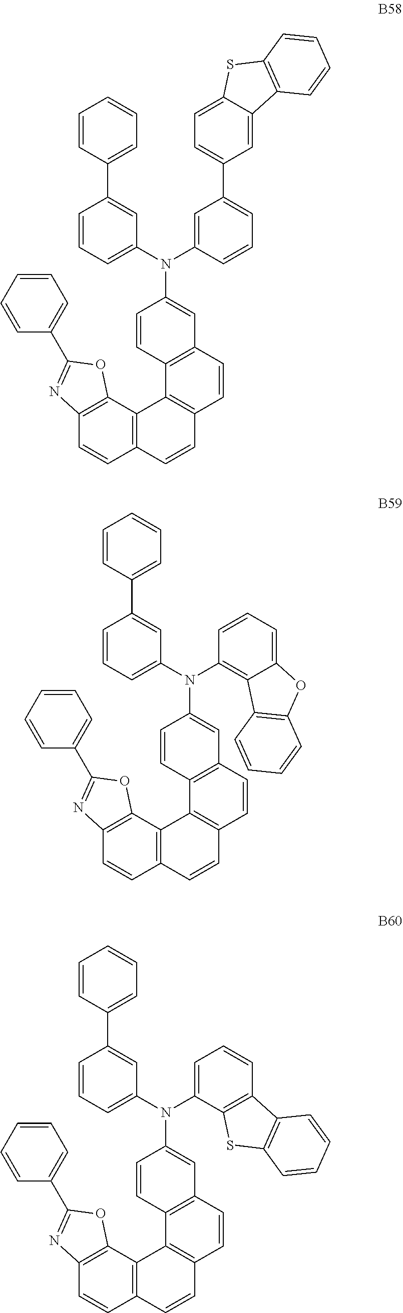 [SMILES: c1ccc(-c2cccc(N(c3cccc(-c4ccc5sc6ccccc6c5c4)c3)c3ccc4c(ccc5ccc6ccc7nc(-c8ccccc8)oc7c6c54)c3)c2)cc1 ; c1ccc(-c2cccc(N(c3ccc4c(ccc5ccc6ccc7nc(-c8ccccc8)oc7c6c54)c3)c3cccc4oc5ccccc5c34)c2)cc1 ; c1ccc(-c2cccc(N(c3ccc4c(ccc5ccc6ccc7nc(-c8ccccc8)oc7c6c54)c3)c3cccc4c3sc3ccccc34)c2)cc1]

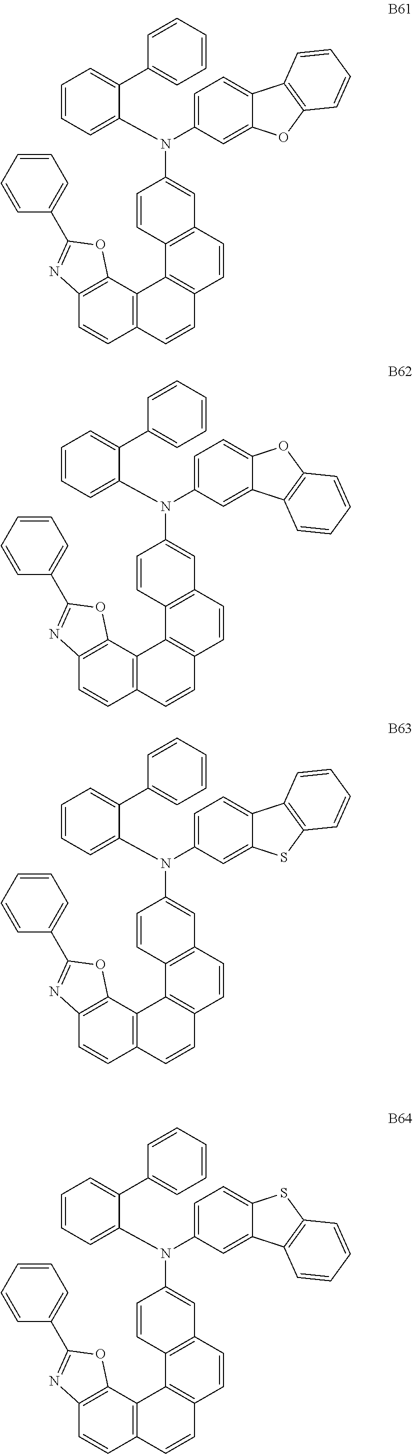

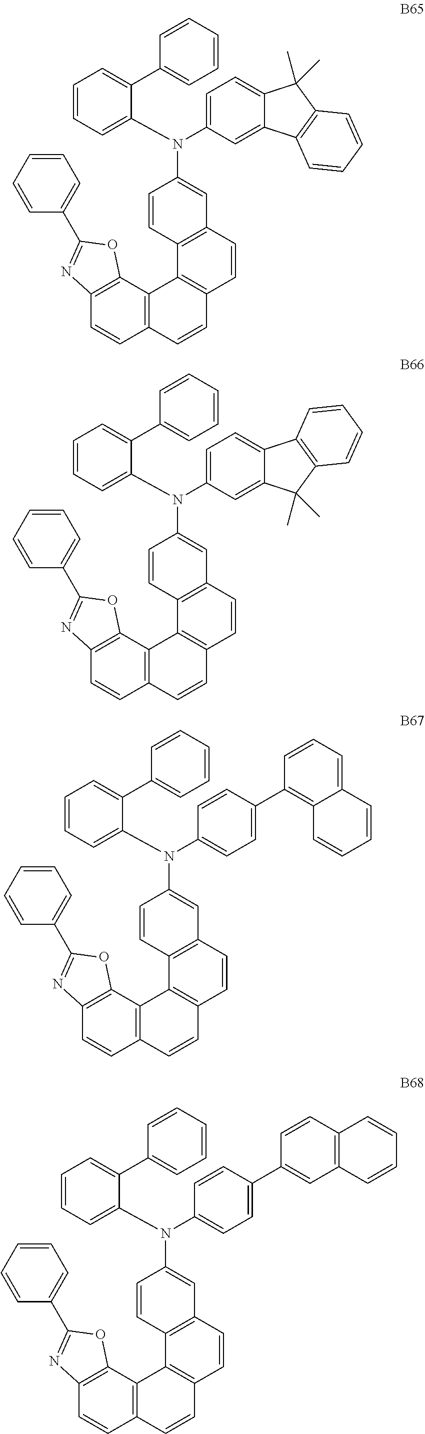

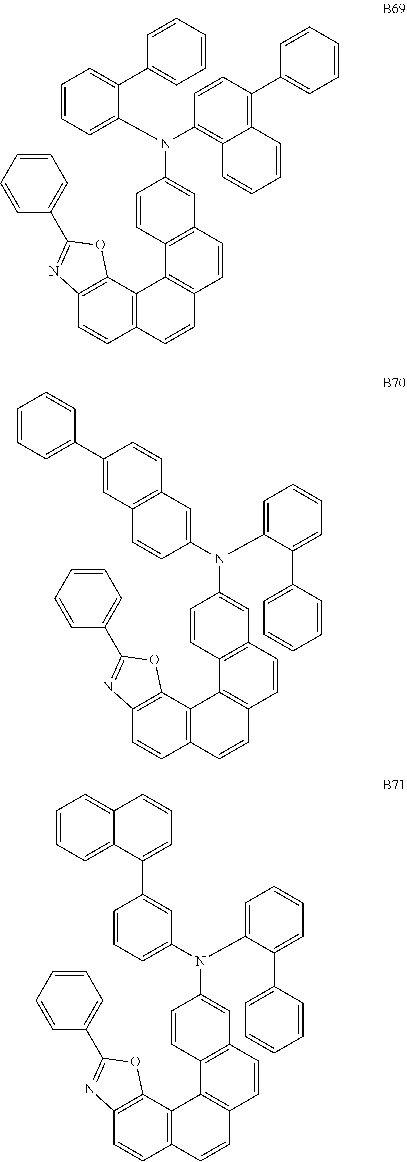

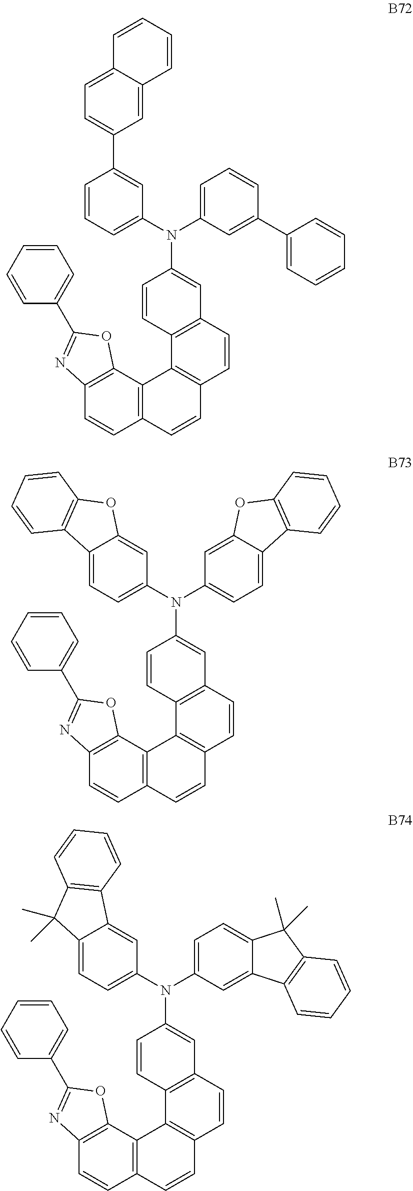

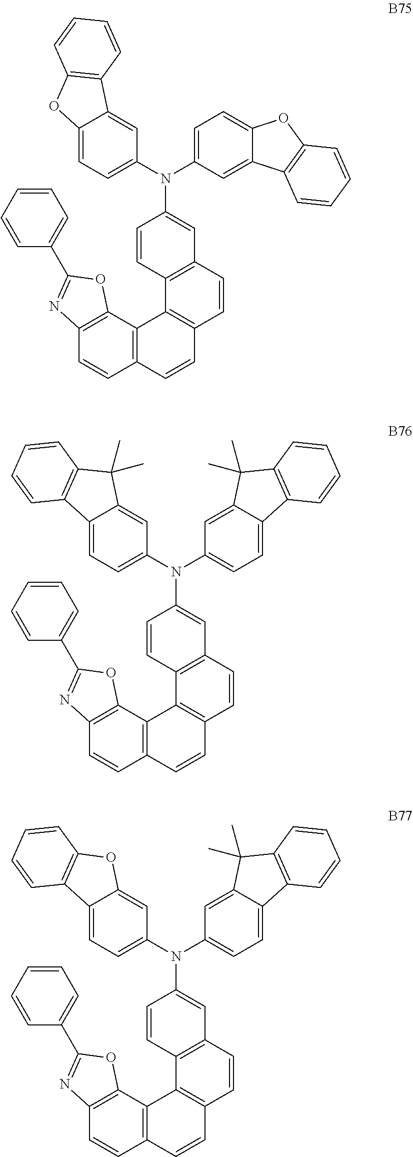 [SMILES: c1ccc(-c2nc3ccc4ccc5ccc6cc(N(c7ccc8oc9ccccc9c8c7)c7ccc8oc9ccccc9c8c7)ccc6c5c4c3o2)cc1 ; CC1(C)c2ccccc2-c2ccc(N(c3ccc4c(c3)C(C)(C)c3ccccc3-4)c3ccc4c(ccc5ccc6ccc7nc(-c8ccccc8)oc7c6c54)c3)cc21 ; CC1(C)c2ccccc2-c2ccc(N(c3ccc4c(ccc5ccc6ccc7nc(-c8ccccc8)oc7c6c54)c3)c3ccc4c(c3)oc3ccccc34)cc21]

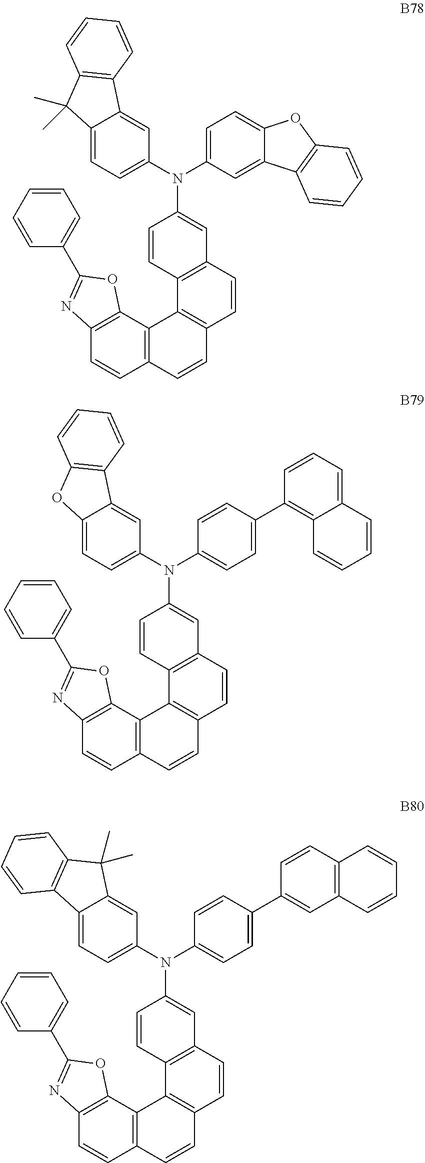

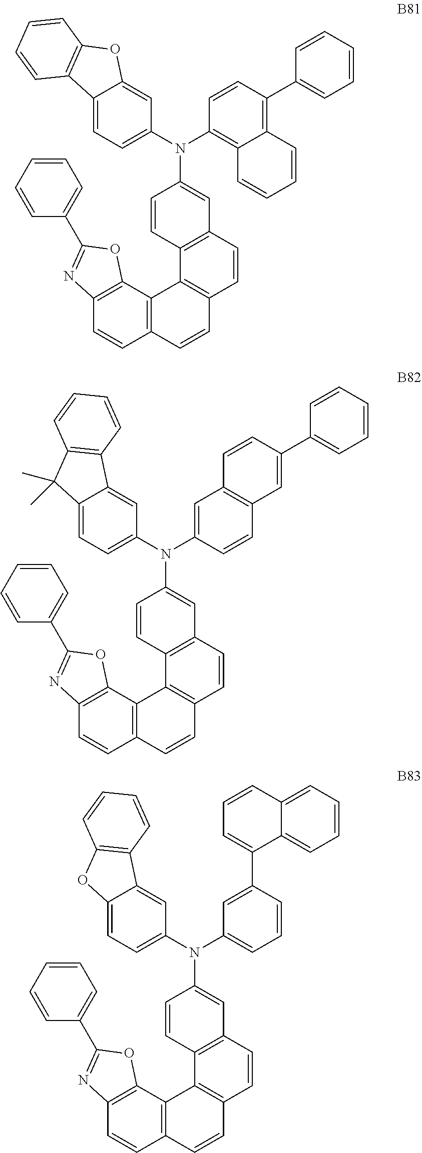

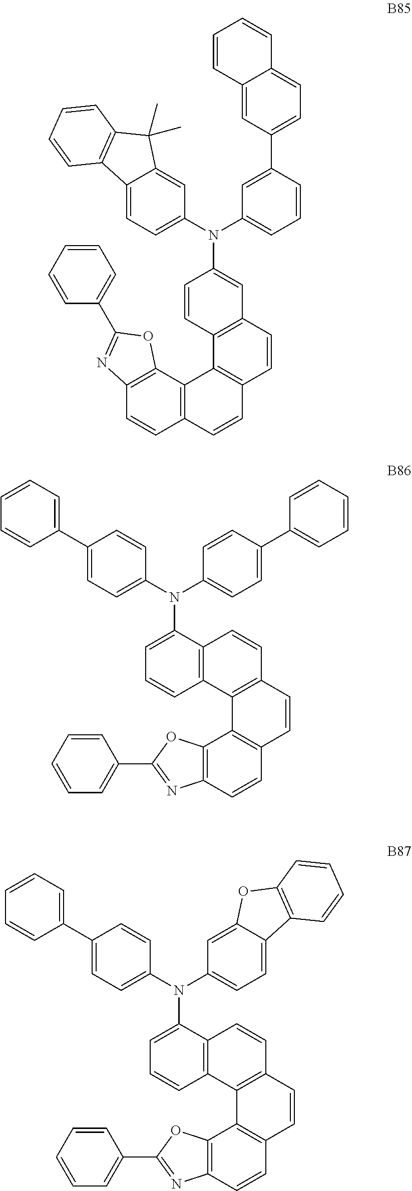 [SMILES: CC1(C)c2ccccc2-c2ccc(N(c3cccc(-c4ccc5ccccc5c4)c3)c3ccc4c(ccc5ccc6ccc7nc(-c8ccccc8)oc7c6c54)c3)cc21 ; c1ccc(-c2ccc(N(c3ccc(-c4ccccc4)cc3)c3cccc4c3ccc3ccc5ccc6nc(-c7ccccc7)oc6c5c34)cc2)cc1 ; c1ccc(-c2ccc(N(c3ccc4c(c3)oc3ccccc34)c3cccc4c3ccc3ccc5ccc6nc(-c7ccccc7)oc6c5c34)cc2)cc1]

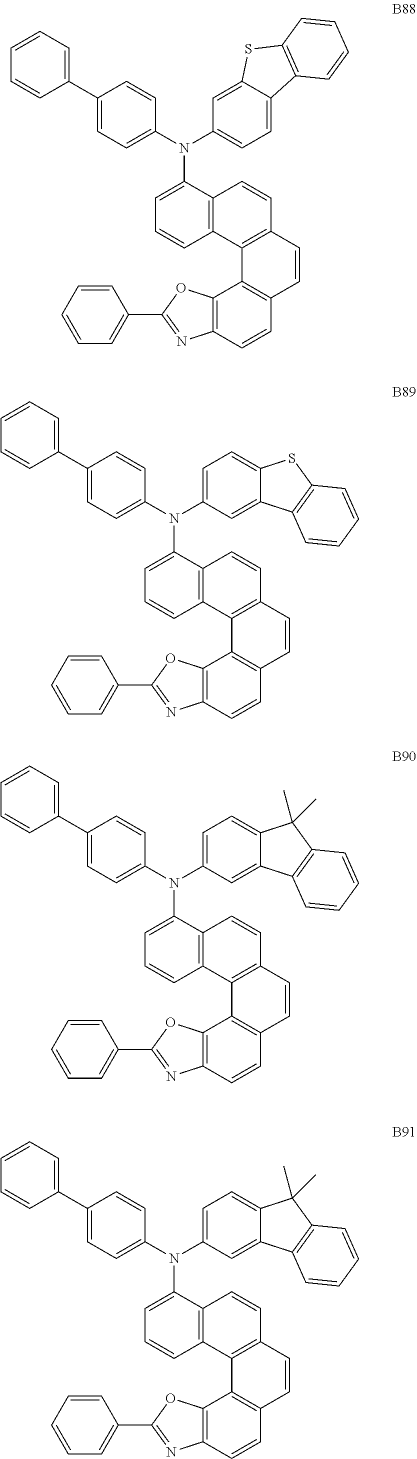 [SMILES: c1ccc(-c2ccc(N(c3ccc4c(c3)sc3ccccc34)c3cccc4c3ccc3ccc5ccc6nc(-c7ccccc7)oc6c5c34)cc2)cc1 ; c1ccc(-c2ccc(N(c3ccc4sc5ccccc5c4c3)c3cccc4c3ccc3ccc5ccc6nc(-c7ccccc7)oc6c5c34)cc2)cc1 ; CC1(C)c2ccccc2-c2cc(N(c3ccc(-c4ccccc4)cc3)c3cccc4c3ccc3ccc5ccc6nc(-c7ccccc7)oc6c5c34)ccc21 ; CC1(C)c2ccccc2-c2cc(N(c3ccc(-c4ccccc4)cc3)c3cccc4c3ccc3ccc5ccc6nc(-c7ccccc7)oc6c5c34)ccc21]

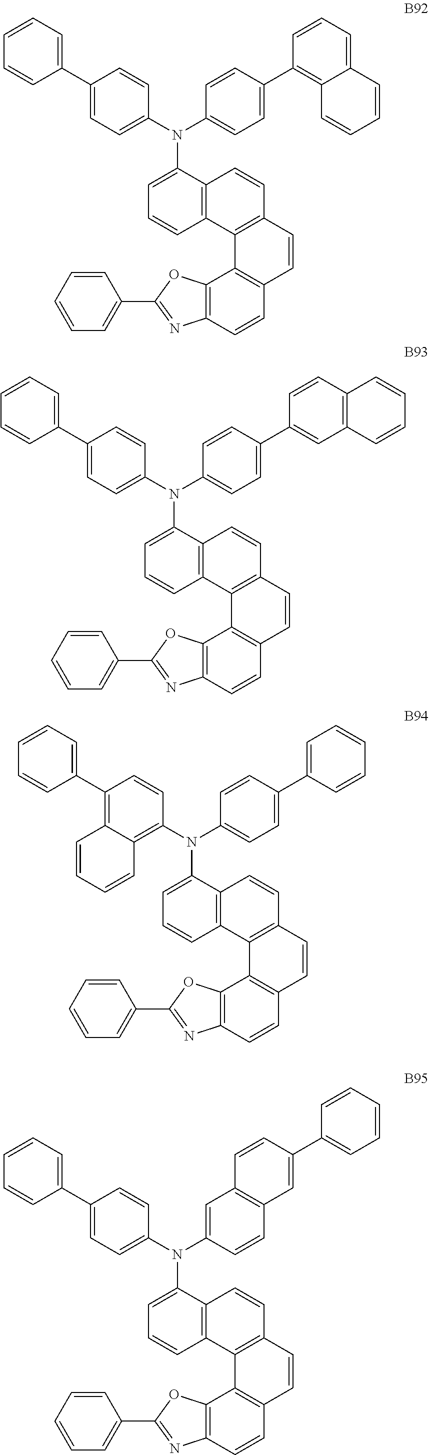

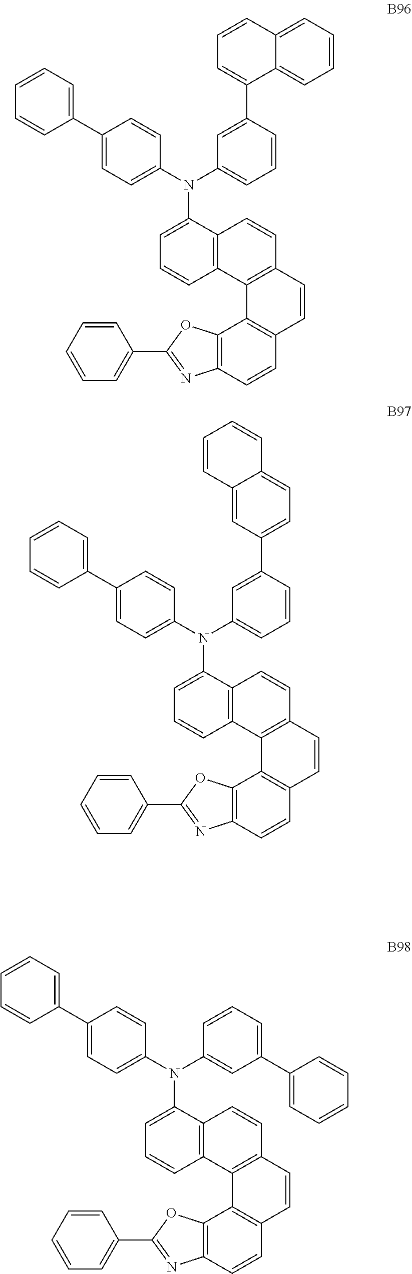

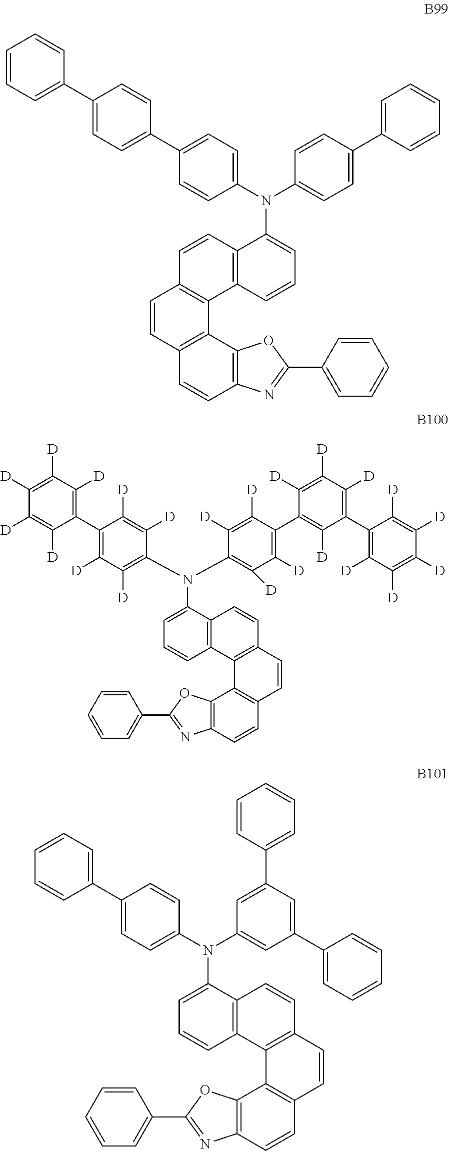 [SMILES: c1ccc(-c2ccc(-c3ccc(N(c4ccc(-c5ccccc5)cc4)c4cccc5c4ccc4ccc6ccc7nc(-c8ccccc8)oc7c6c45)cc3)cc2)cc1 ; [2H]c1c([2H])c([2H])c(-c2c([2H])c([2H])c(N(c3c([2H])c([2H])c(-c4c([2H])c([2H])c([2H])c(-c5c([2H])c([2H])c([2H])c([2H])c5[2H])c4[2H])c([2H])c3[2H])c3cccc4c3ccc3ccc5ccc6nc(-c7ccccc7)oc6c5c34)c([2H])c2[2H])c([2H])c1[2H] ; c1ccc(-c2ccc(N(c3cc(-c4ccccc4)cc(-c4ccccc4)c3)c3cccc4c3ccc3ccc5ccc6nc(-c7ccccc7)oc6c5c34)cc2)cc1]

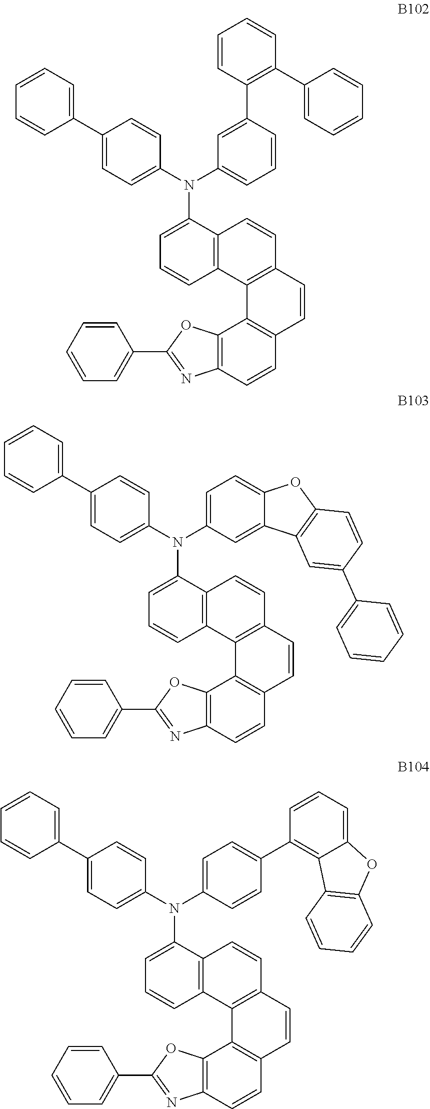 [SMILES: c1ccc(-c2ccc(N(c3cccc(-c4ccccc4-c4ccccc4)c3)c3cccc4c3ccc3ccc5ccc6nc(-c7ccccc7)oc6c5c34)cc2)cc1 ; c1ccc(-c2ccc(N(c3ccc4oc5ccc(-c6ccccc6)cc5c4c3)c3cccc4c3ccc3ccc5ccc6nc(-c7ccccc7)oc6c5c34)cc2)cc1 ; c1ccc(-c2ccc(N(c3ccc(-c4cccc5oc6ccccc6c45)cc3)c3cccc4c3ccc3ccc5ccc6nc(-c7ccccc7)oc6c5c34)cc2)cc1]

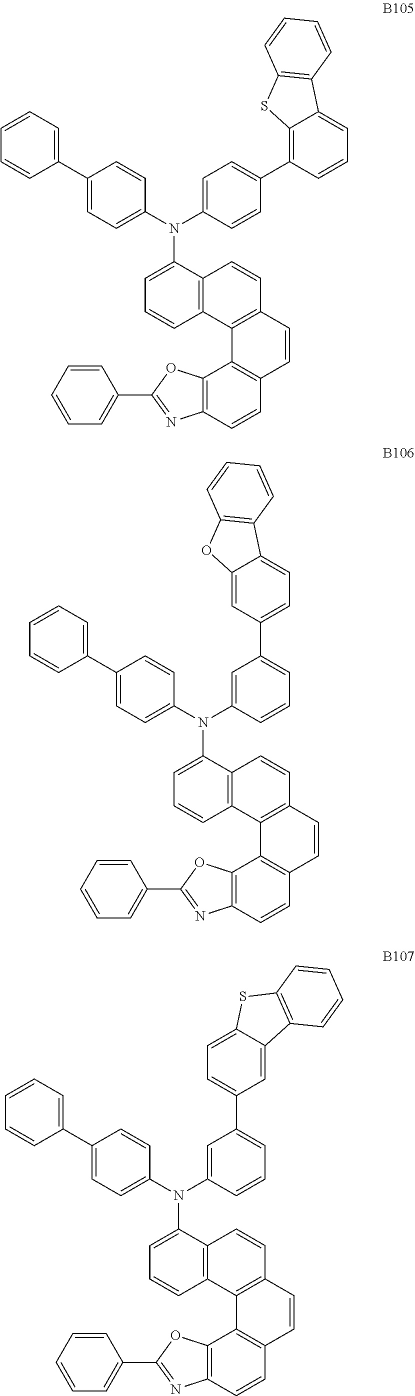

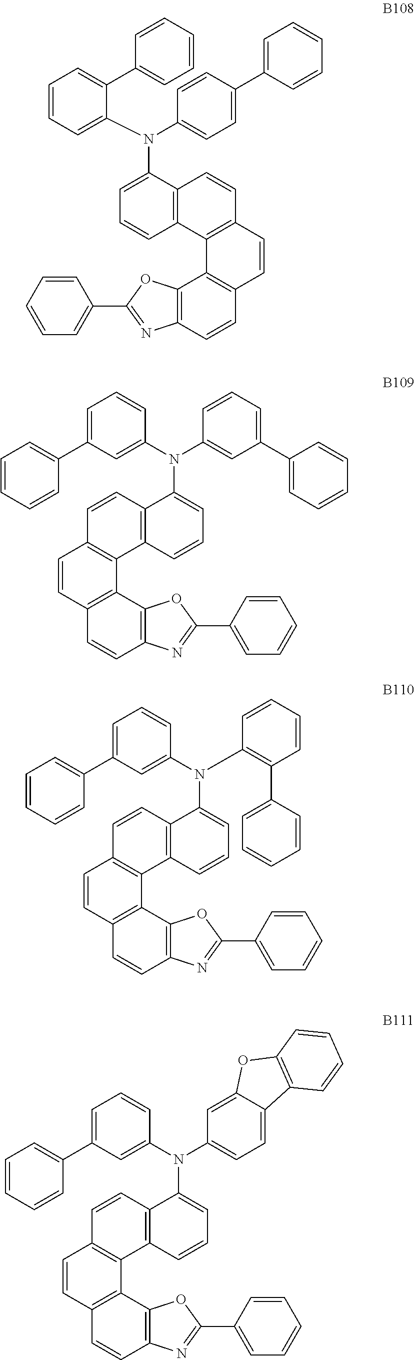

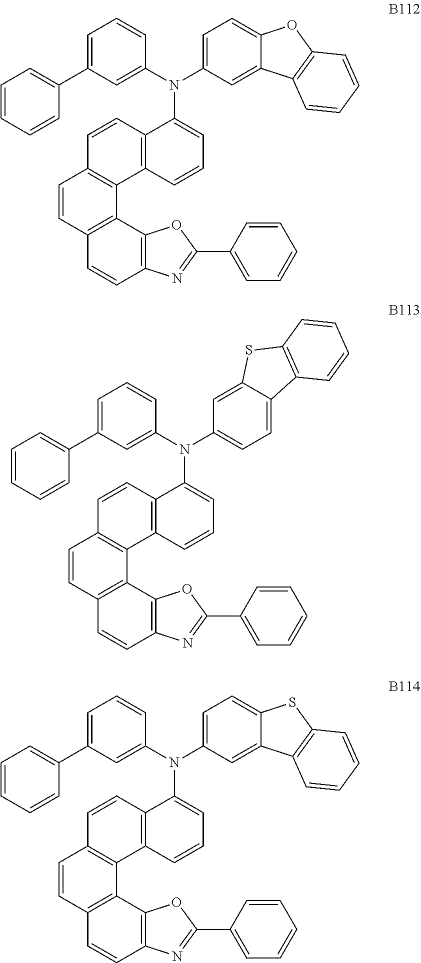

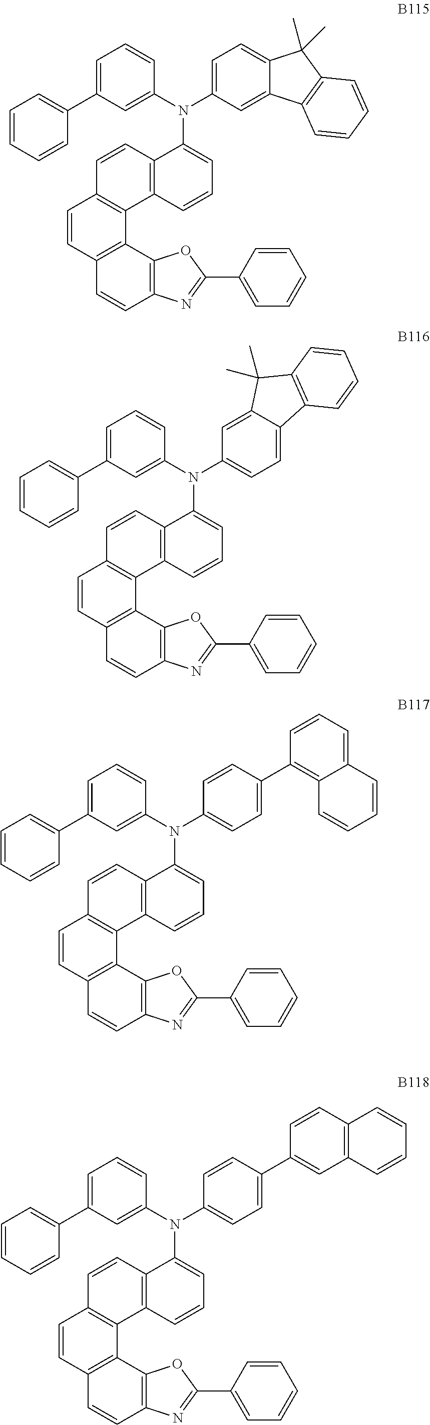

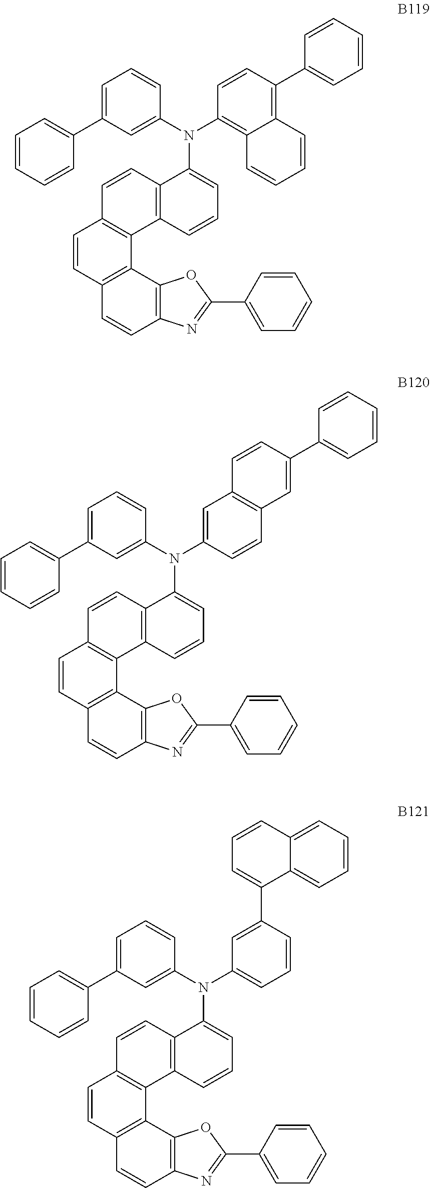 [SMILES: c1ccc(-c2cccc(N(c3ccc(-c4ccccc4)c4ccccc34)c3cccc4c3ccc3ccc5ccc6nc(-c7ccccc7)oc6c5c34)c2)cc1 ; c1ccc(-c2cccc(N(c3ccc4cc(-c5ccccc5)ccc4c3)c3cccc4c3ccc3ccc5ccc6nc(-c7ccccc7)oc6c5c34)c2)cc1 ; c1ccc(-c2cccc(N(c3cccc(-c4cccc5ccccc45)c3)c3cccc4c3ccc3ccc5ccc6nc(-c7ccccc7)oc6c5c34)c2)cc1]

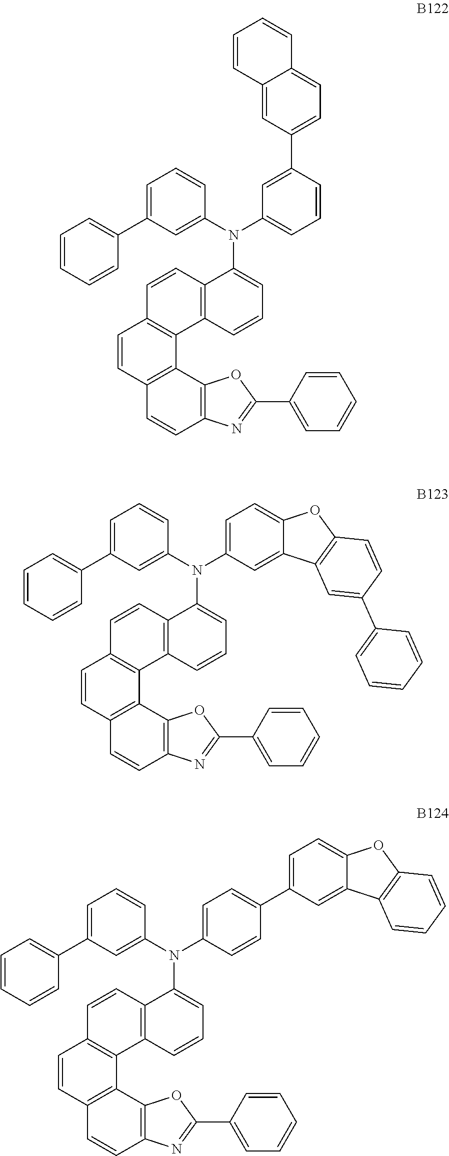 [SMILES: c1ccc(-c2cccc(N(c3cccc(-c4ccc5ccccc5c4)c3)c3cccc4c3ccc3ccc5ccc6nc(-c7ccccc7)oc6c5c34)c2)cc1 ; c1ccc(-c2cccc(N(c3ccc4oc5ccc(-c6ccccc6)cc5c4c3)c3cccc4c3ccc3ccc5ccc6nc(-c7ccccc7)oc6c5c34)c2)cc1 ; c1ccc(-c2cccc(N(c3ccc(-c4ccc5oc6ccccc6c5c4)cc3)c3cccc4c3ccc3ccc5ccc6nc(-c7ccccc7)oc6c5c34)c2)cc1]

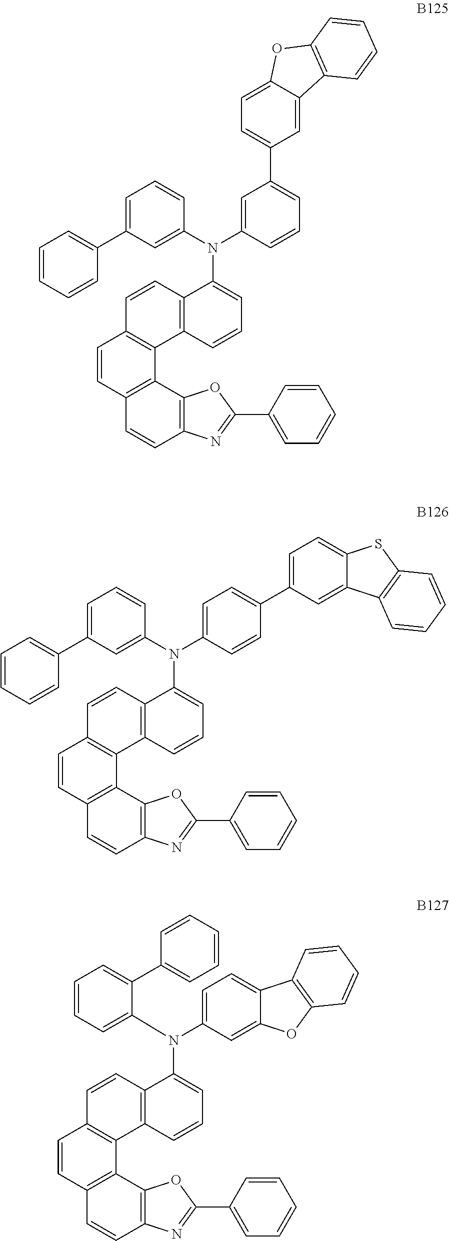 [SMILES: c1ccc(-c2cccc(N(c3cccc(-c4ccc5oc6ccccc6c5c4)c3)c3cccc4c3ccc3ccc5ccc6nc(-c7ccccc7)oc6c5c34)c2)cc1 ; c1ccc(-c2cccc(N(c3ccc(-c4ccc5sc6ccccc6c5c4)cc3)c3cccc4c3ccc3ccc5ccc6nc(-c7ccccc7)oc6c5c34)c2)cc1 ; c1ccc(-c2nc3ccc4ccc5ccc6c(N(c7ccc8c(c7)oc7ccccc78)c7ccccc7-c7ccccc7)cccc6c5c4c3o2)cc1]

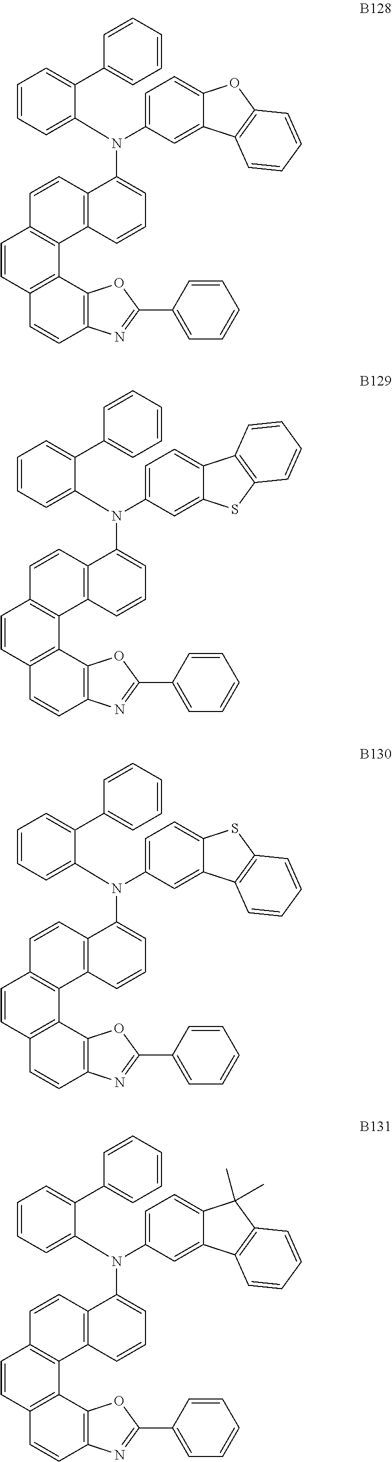 [SMILES: c1ccc(-c2nc3ccc4ccc5ccc6c(N(c7ccc8oc9ccccc9c8c7)c7ccccc7-c7ccccc7)cccc6c5c4c3o2)cc1 ; c1ccc(-c2nc3ccc4ccc5ccc6c(N(c7ccc8c(c7)sc7ccccc78)c7ccccc7-c7ccccc7)cccc6c5c4c3o2)cc1 ; c1ccc(-c2nc3ccc4ccc5ccc6c(N(c7ccc8sc9ccccc9c8c7)c7ccccc7-c7ccccc7)cccc6c5c4c3o2)cc1 ; CC1(C)c2ccccc2-c2cc(N(c3ccccc3-c3ccccc3)c3cccc4c3ccc3ccc5ccc6nc(-c7ccccc7)oc6c5c34)ccc21]

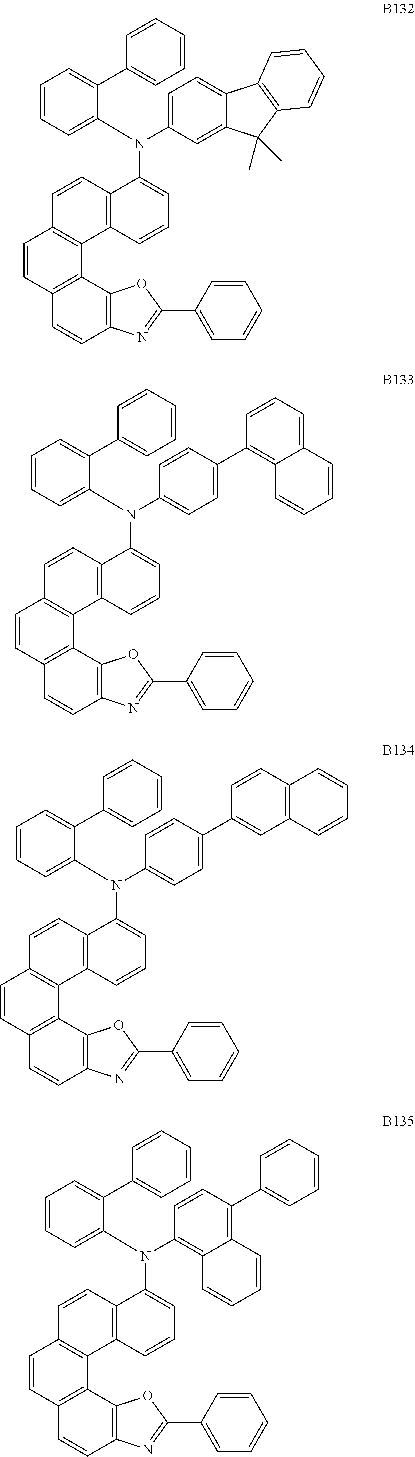 [SMILES: CC1(C)c2ccccc2-c2ccc(N(c3ccccc3-c3ccccc3)c3cccc4c3ccc3ccc5ccc6nc(-c7ccccc7)oc6c5c34)cc21 ; c1ccc(-c2nc3ccc4ccc5ccc6c(N(c7ccc(-c8cccc9ccccc89)cc7)c7ccccc7-c7ccccc7)cccc6c5c4c3o2)cc1 ; c1ccc(-c2nc3ccc4ccc5ccc6c(N(c7ccc(-c8ccc9ccccc9c8)cc7)c7ccccc7-c7ccccc7)cccc6c5c4c3o2)cc1 ; c1ccc(-c2nc3ccc4ccc5ccc6c(N(c7ccccc7-c7ccccc7)c7ccc(-c8ccccc8)c8ccccc78)cccc6c5c4c3o2)cc1]

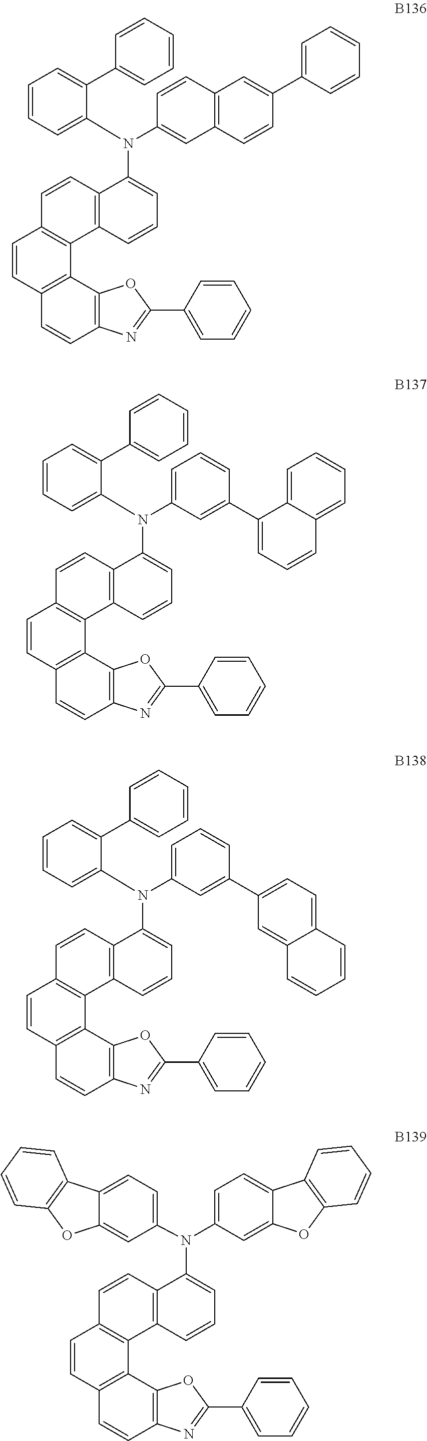 [SMILES: c1ccc(-c2ccc3cc(N(c4ccccc4-c4ccccc4)c4cccc5c4ccc4ccc6ccc7nc(-c8ccccc8)oc7c6c45)ccc3c2)cc1 ; c1ccc(-c2nc3ccc4ccc5ccc6c(N(c7cccc(-c8cccc9ccccc89)c7)c7ccccc7-c7ccccc7)cccc6c5c4c3o2)cc1 ; c1ccc(-c2nc3ccc4ccc5ccc6c(N(c7cccc(-c8ccc9ccccc9c8)c7)c7ccccc7-c7ccccc7)cccc6c5c4c3o2)cc1 ; c1ccc(-c2nc3ccc4ccc5ccc6c(N(c7ccc8c(c7)oc7ccccc78)c7ccc8c(c7)oc7ccccc78)cccc6c5c4c3o2)cc1]

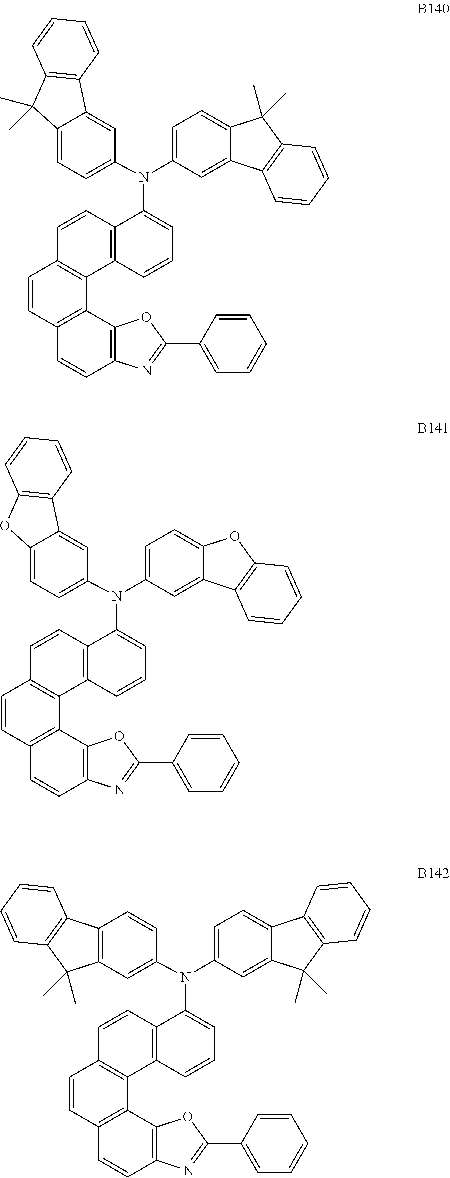 [SMILES: CC1(C)c2ccccc2-c2cc(N(c3ccc4c(c3)-c3ccccc3C4(C)C)c3cccc4c3ccc3ccc5ccc6nc(-c7ccccc7)oc6c5c34)ccc21 ; c1ccc(-c2nc3ccc4ccc5ccc6c(N(c7ccc8oc9ccccc9c8c7)c7ccc8oc9ccccc9c8c7)cccc6c5c4c3o2)cc1 ; CC1(C)c2ccccc2-c2ccc(N(c3ccc4c(c3)C(C)(C)c3ccccc3-4)c3cccc4c3ccc3ccc5ccc6nc(-c7ccccc7)oc6c5c34)cc21]

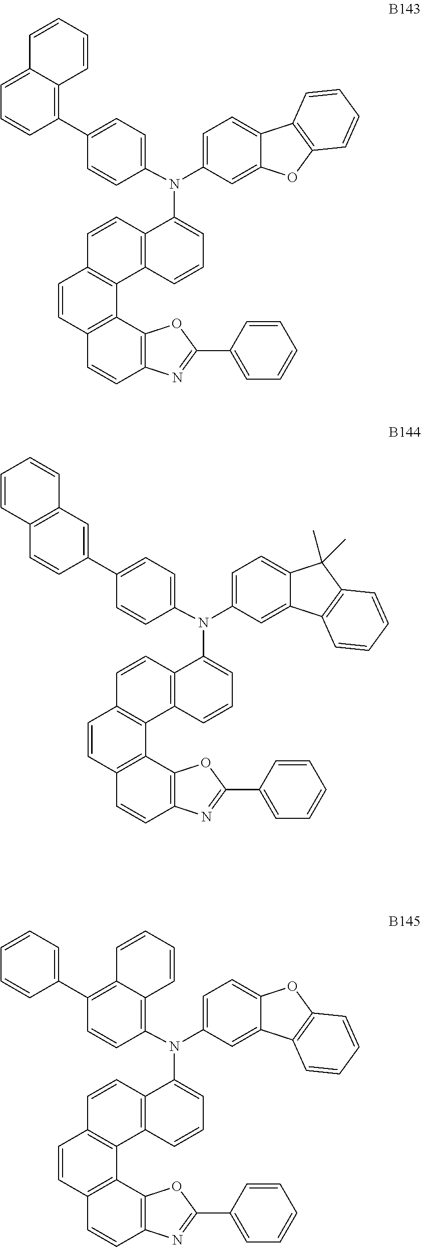 [SMILES: c1ccc(-c2nc3ccc4ccc5ccc6c(N(c7ccc(-c8cccc9ccccc89)cc7)c7ccc8c(c7)oc7ccccc78)cccc6c5c4c3o2)cc1 ; CC1(C)c2ccccc2-c2cc(N(c3ccc(-c4ccc5ccccc5c4)cc3)c3cccc4c3ccc3ccc5ccc6nc(-c7ccccc7)oc6c5c34)ccc21 ; c1ccc(-c2nc3ccc4ccc5ccc6c(N(c7ccc8oc9ccccc9c8c7)c7ccc(-c8ccccc8)c8ccccc78)cccc6c5c4c3o2)cc1]

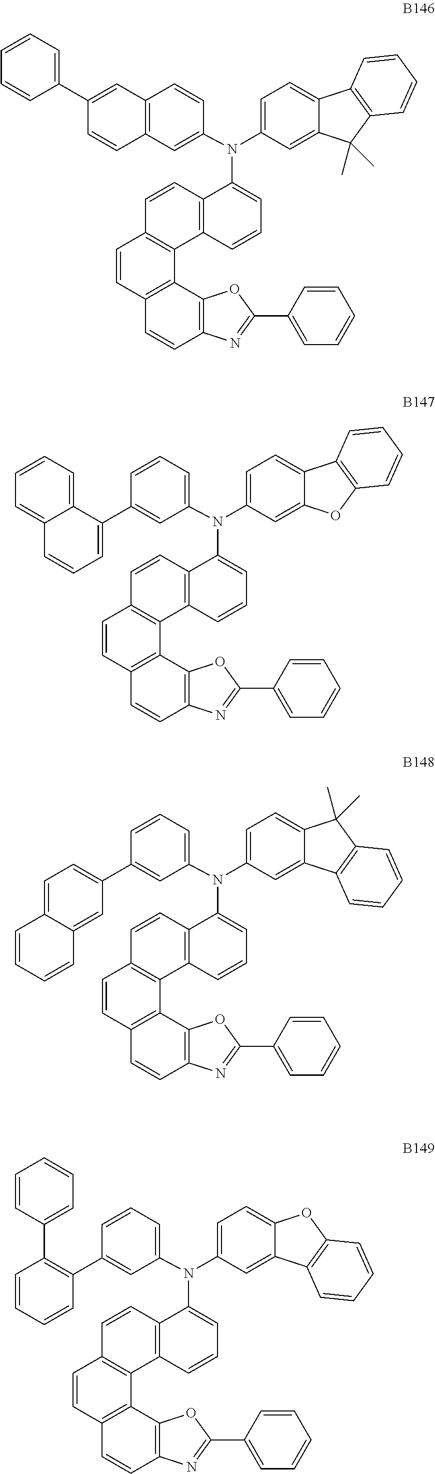 [SMILES: CC1(C)c2ccccc2-c2ccc(N(c3ccc4cc(-c5ccccc5)ccc4c3)c3cccc4c3ccc3ccc5ccc6nc(-c7ccccc7)oc6c5c34)cc21 ; c1ccc(-c2nc3ccc4ccc5ccc6c(N(c7cccc(-c8cccc9ccccc89)c7)c7ccc8c(c7)oc7ccccc78)cccc6c5c4c3o2)cc1 ; CC1(C)c2ccccc2-c2cc(N(c3cccc(-c4ccc5ccccc5c4)c3)c3cccc4c3ccc3ccc5ccc6nc(-c7ccccc7)oc6c5c34)ccc21 ; c1ccc(-c2nc3ccc4ccc5ccc6c(N(c7cccc(-c8ccccc8-c8ccccc8)c7)c7ccc8oc9ccccc9c8c7)cccc6c5c4c3o2)cc1]

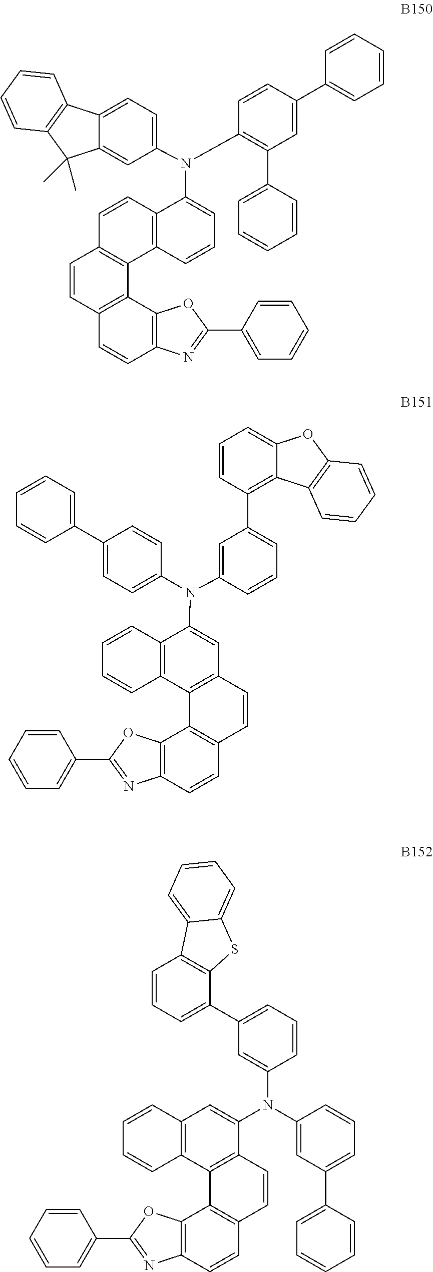 [SMILES: CC1(C)c2ccccc2-c2ccc(N(c3ccc(-c4ccccc4)cc3-c3ccccc3)c3cccc4c3ccc3ccc5ccc6nc(-c7ccccc7)oc6c5c34)cc21 ; c1ccc(-c2ccc(N(c3cccc(-c4cccc5oc6ccccc6c45)c3)c3cc4ccc5ccc6nc(-c7ccccc7)oc6c5c4c4ccccc34)cc2)cc1 ; c1ccc(-c2cccc(N(c3cccc(-c4cccc5c4sc4ccccc45)c3)c3cc4ccccc4c4c3ccc3ccc5nc(-c6ccccc6)oc5c34)c2)cc1]

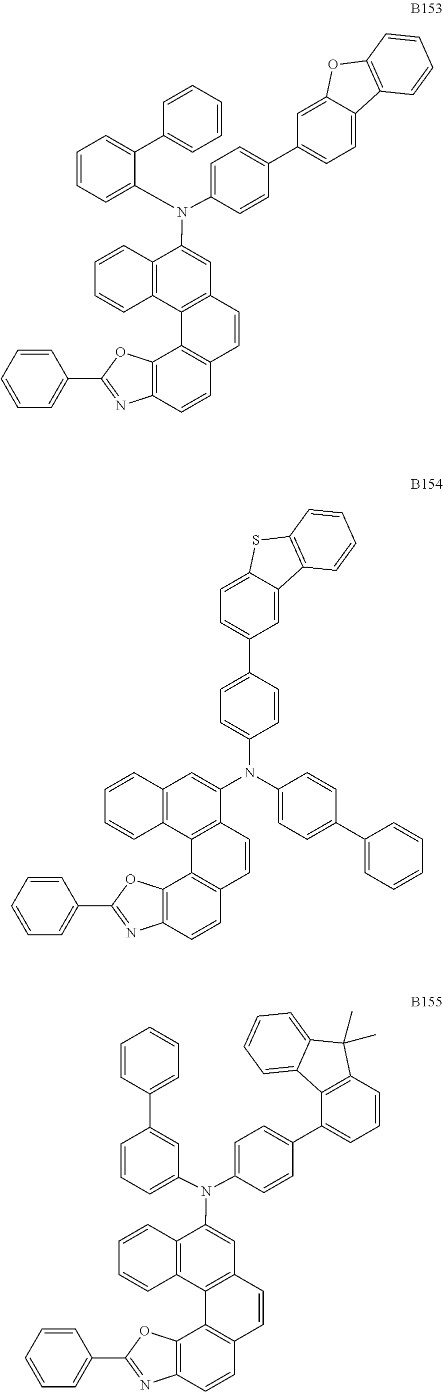 [SMILES: c1ccc(-c2nc3ccc4ccc5cc(N(c6ccc(-c7ccc8c(c7)oc7ccccc78)cc6)c6ccccc6-c6ccccc6)c6ccccc6c5c4c3o2)cc1 ; c1ccc(-c2ccc(N(c3ccc(-c4ccc5sc6ccccc6c5c4)cc3)c3cc4ccccc4c4c3ccc3ccc5nc(-c6ccccc6)oc5c34)cc2)cc1 ; CC1(C)c2ccccc2-c2c(-c3ccc(N(c4cccc(-c5ccccc5)c4)c4cc5ccc6ccc7nc(-c8ccccc8)oc7c6c5c5ccccc45)cc3)cccc21]

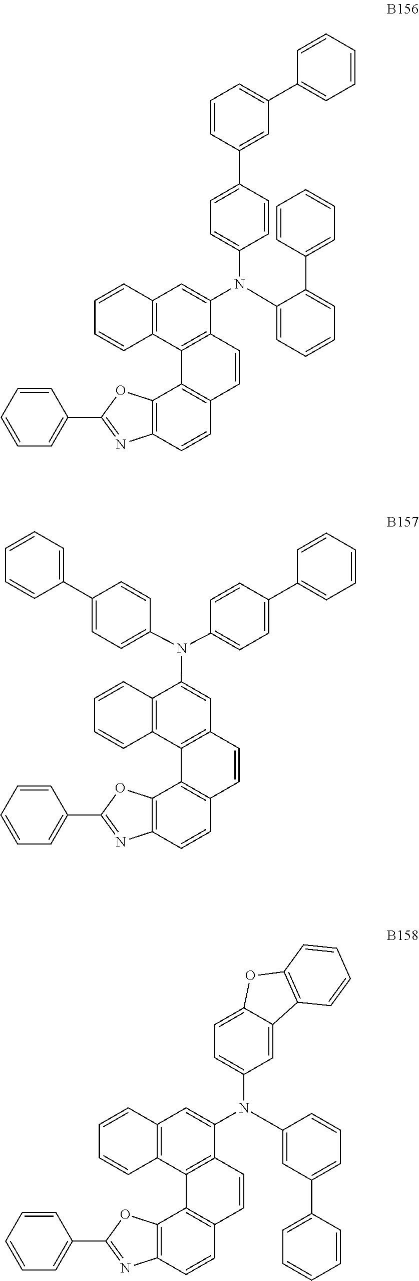 [SMILES: c1ccc(-c2cccc(-c3ccc(N(c4ccccc4-c4ccccc4)c4cc5ccccc5c5c4ccc4ccc6nc(-c7ccccc7)oc6c45)cc3)c2)cc1 ; c1ccc(-c2ccc(N(c3ccc(-c4ccccc4)cc3)c3cc4ccc5ccc6nc(-c7ccccc7)oc6c5c4c4ccccc34)cc2)cc1 ; c1ccc(-c2cccc(N(c3ccc4oc5ccccc5c4c3)c3cc4ccccc4c4c3ccc3ccc5nc(-c6ccccc6)oc5c34)c2)cc1]

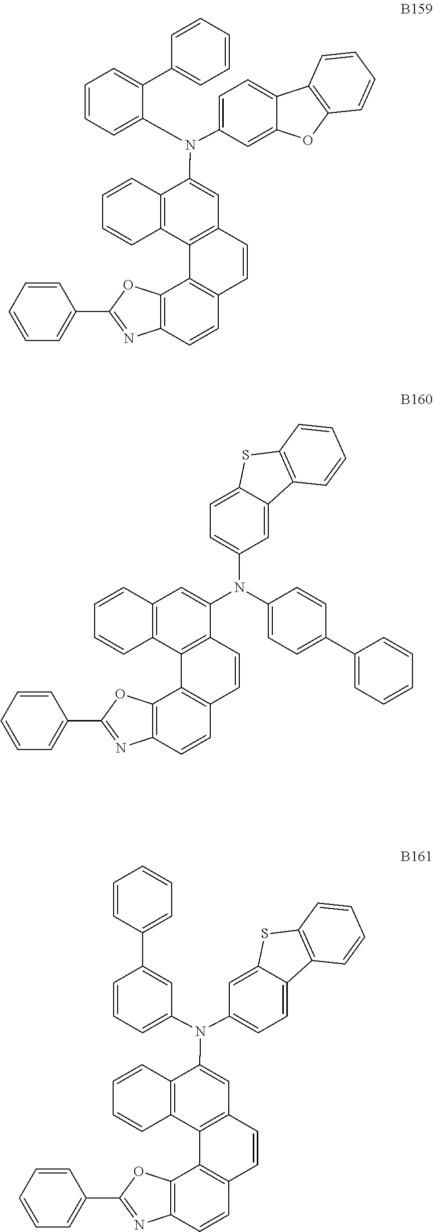 [SMILES: c1ccc(-c2nc3ccc4ccc5cc(N(c6ccc7c(c6)oc6ccccc67)c6ccccc6-c6ccccc6)c6ccccc6c5c4c3o2)cc1 ; c1ccc(-c2ccc(N(c3ccc4sc5ccccc5c4c3)c3cc4ccccc4c4c3ccc3ccc5nc(-c6ccccc6)oc5c34)cc2)cc1 ; c1ccc(-c2cccc(N(c3ccc4c(c3)sc3ccccc34)c3cc4ccc5ccc6nc(-c7ccccc7)oc6c5c4c4ccccc34)c2)cc1]

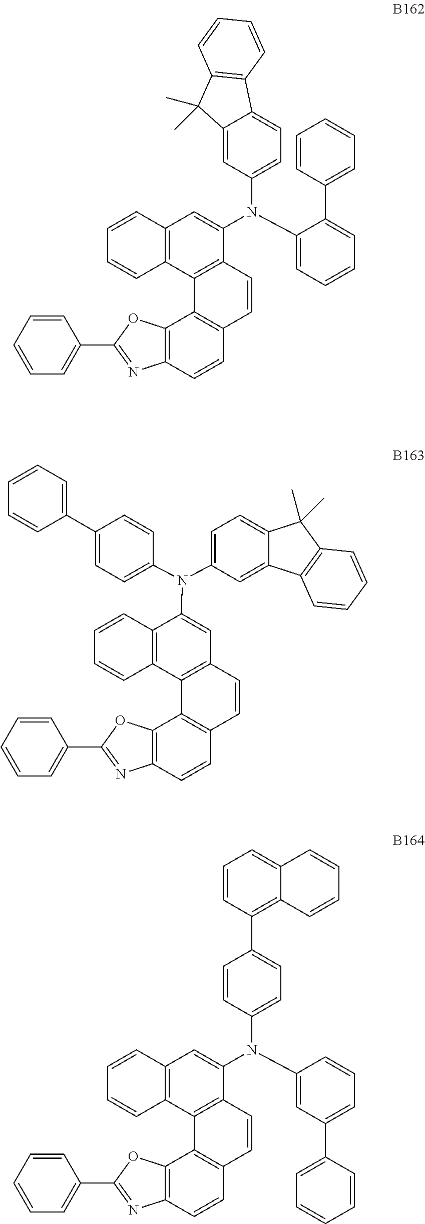 [SMILES: CC1(C)c2ccccc2-c2ccc(N(c3ccccc3-c3ccccc3)c3cc4ccccc4c4c3ccc3ccc5nc(-c6ccccc6)oc5c34)cc21 ; CC1(C)c2ccccc2-c2cc(N(c3ccc(-c4ccccc4)cc3)c3cc4ccc5ccc6nc(-c7ccccc7)oc6c5c4c4ccccc34)ccc21 ; c1ccc(-c2cccc(N(c3ccc(-c4cccc5ccccc45)cc3)c3cc4ccccc4c4c3ccc3ccc5nc(-c6ccccc6)oc5c34)c2)cc1]

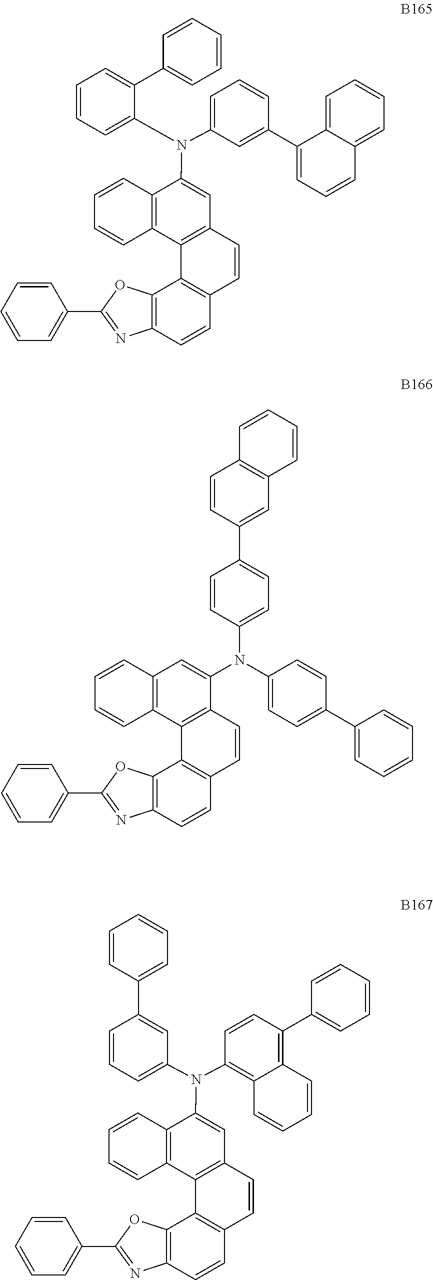 [SMILES: c1ccc(-c2nc3ccc4ccc5cc(N(c6cccc(-c7cccc8ccccc78)c6)c6ccccc6-c6ccccc6)c6ccccc6c5c4c3o2)cc1 ; c1ccc(-c2ccc(N(c3ccc(-c4ccc5ccccc5c4)cc3)c3cc4ccccc4c4c3ccc3ccc5nc(-c6ccccc6)oc5c34)cc2)cc1 ; c1ccc(-c2cccc(N(c3ccc(-c4ccccc4)c4ccccc34)c3cc4ccc5ccc6nc(-c7ccccc7)oc6c5c4c4ccccc34)c2)cc1]

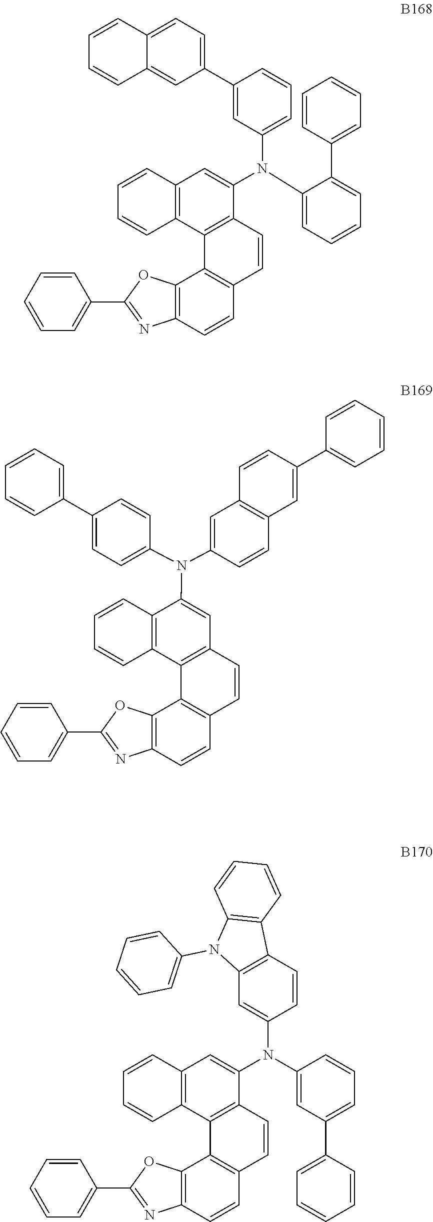 [SMILES: c1ccc(-c2nc3ccc4ccc5c(N(c6cccc(-c7ccc8ccccc8c7)c6)c6ccccc6-c6ccccc6)cc6ccccc6c5c4c3o2)cc1 ; c1ccc(-c2ccc(N(c3ccc4cc(-c5ccccc5)ccc4c3)c3cc4ccc5ccc6nc(-c7ccccc7)oc6c5c4c4ccccc34)cc2)cc1 ; c1ccc(-c2cccc(N(c3ccc4c5ccccc5n(-c5ccccc5)c4c3)c3cc4ccccc4c4c3ccc3ccc5nc(-c6ccccc6)oc5c34)c2)cc1]

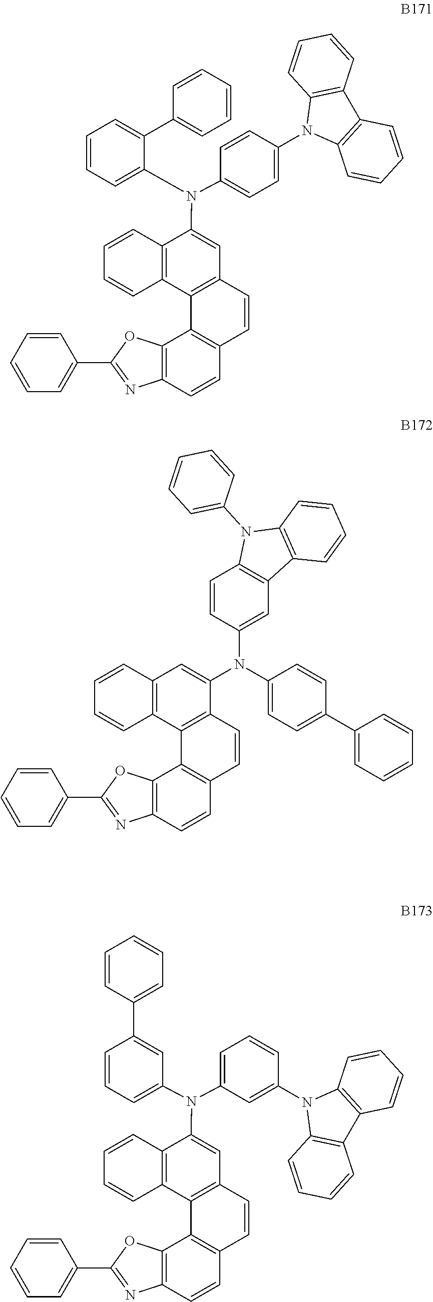 [SMILES: c1ccc(-c2nc3ccc4ccc5cc(N(c6ccc(-n7c8ccccc8c8ccccc87)cc6)c6ccccc6-c6ccccc6)c6ccccc6c5c4c3o2)cc1 ; c1ccc(-c2ccc(N(c3ccc4c(c3)c3ccccc3n4-c3ccccc3)c3cc4ccccc4c4c3ccc3ccc5nc(-c6ccccc6)oc5c34)cc2)cc1 ; c1ccc(-c2cccc(N(c3cccc(-n4c5ccccc5c5ccccc54)c3)c3cc4ccc5ccc6nc(-c7ccccc7)oc6c5c4c4ccccc34)c2)cc1]

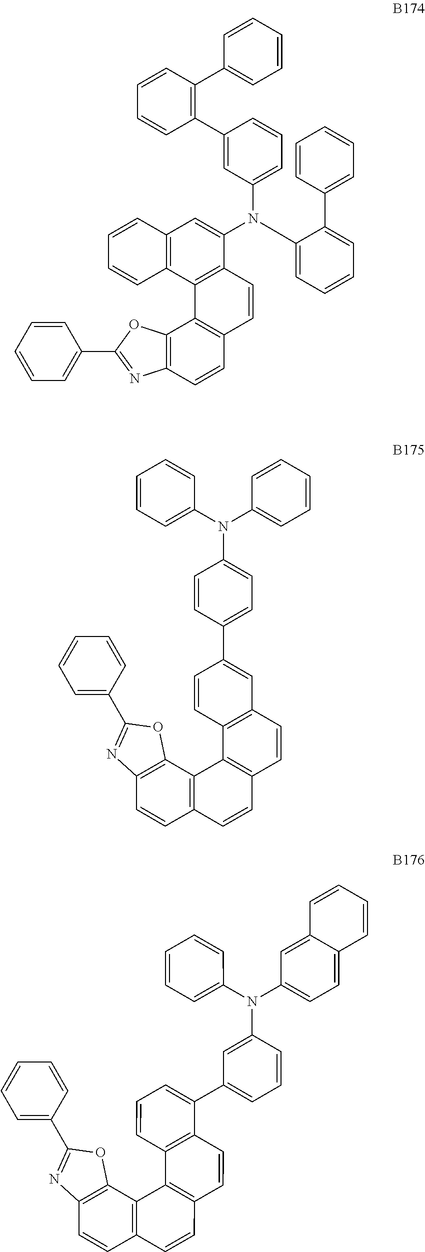 [SMILES: c1ccc(-c2nc3ccc4ccc5c(N(c6cccc(-c7ccccc7-c7ccccc7)c6)c6ccccc6-c6ccccc6)cc6ccccc6c5c4c3o2)cc1 ; c1ccc(-c2nc3ccc4ccc5ccc6cc(-c7ccc(N(c8ccccc8)c8ccccc8)cc7)ccc6c5c4c3o2)cc1 ; c1ccc(-c2nc3ccc4ccc5ccc6c(-c7cccc(N(c8ccccc8)c8ccc9ccccc9c8)c7)cccc6c5c4c3o2)cc1]

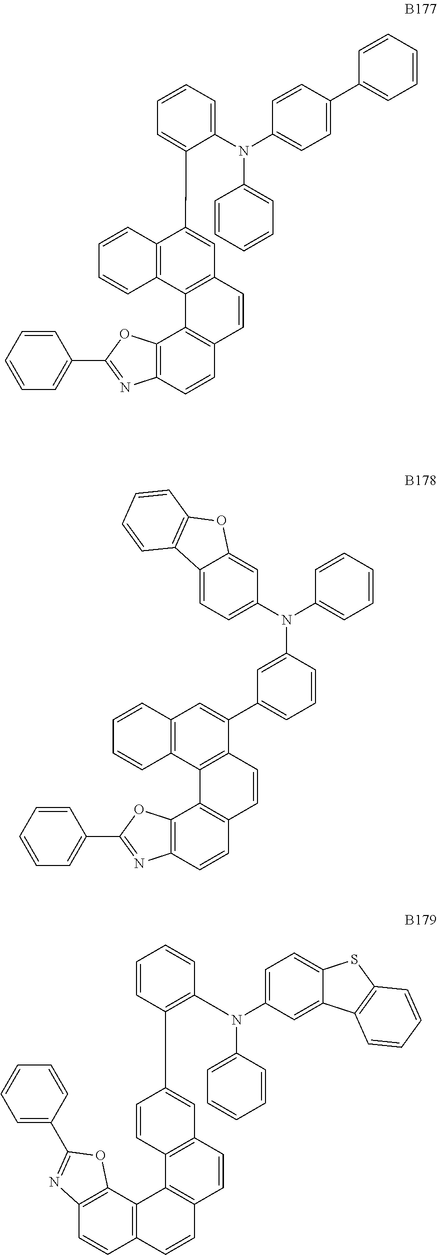 [SMILES: c1ccc(-c2ccc(N(c3ccccc3)c3ccccc3-c3cc4ccc5ccc6nc(-c7ccccc7)oc6c5c4c4ccccc34)cc2)cc1 ; c1ccc(-c2nc3ccc4ccc5c(-c6cccc(N(c7ccccc7)c7ccc8c(c7)oc7ccccc78)c6)cc6ccccc6c5c4c3o2)cc1 ; c1ccc(-c2nc3ccc4ccc5ccc6cc(-c7ccccc7CN(c7ccccc7)c7ccc8sc9ccccc9c8c7)ccc6c5c4c3o2)cc1]

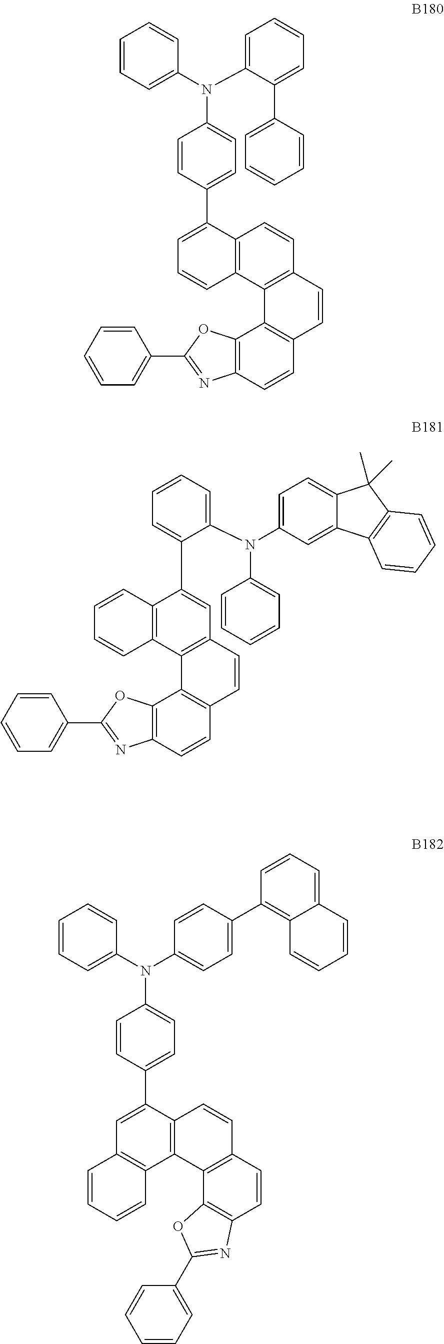 [SMILES: c1ccc(-c2nc3ccc4ccc5ccc6c(-c7ccc(N(c8ccccc8)c8ccccc8-c8ccccc8)cc7)cccc6c5c4c3o2)cc1 ; CC1(C)c2ccccc2-c2cc(N(c3ccccc3)c3ccccc3-c3cc4ccc5ccc6nc(-c7ccccc7)oc6c5c4c4ccccc34)ccc21 ; c1ccc(-c2nc3ccc4ccc5c(-c6ccc(N(c7ccccc7)c7ccc(-c8cccc9ccccc89)cc7)cc6)cc6ccccc6c5c4c3o2)cc1]

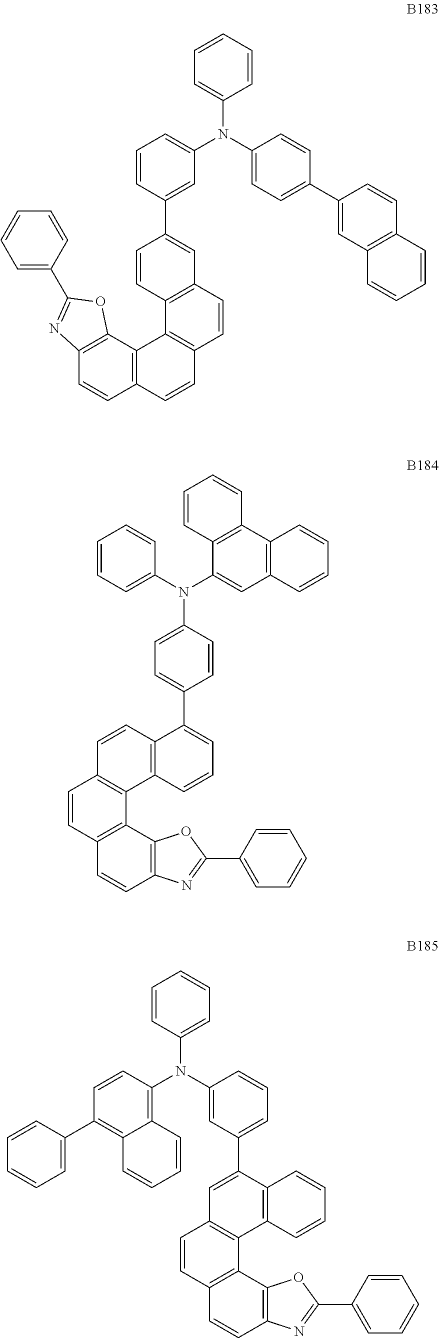 [SMILES: c1ccc(-c2nc3ccc4ccc5ccc6cc(-c7cccc(N(c8ccccc8)c8ccc(-c9ccc%10ccccc%10c9)cc8)c7)ccc6c5c4c3o2)cc1 ; c1ccc(-c2nc3ccc4ccc5ccc6c(-c7ccc(N(c8ccccc8)c8cc9ccccc9c9ccccc89)cc7)cccc6c5c4c3o2)cc1 ; c1ccc(-c2nc3ccc4ccc5cc(-c6cccc(N(c7ccccc7)c7ccc(-c8ccccc8)c8ccccc78)c6)c6ccccc6c5c4c3o2)cc1]

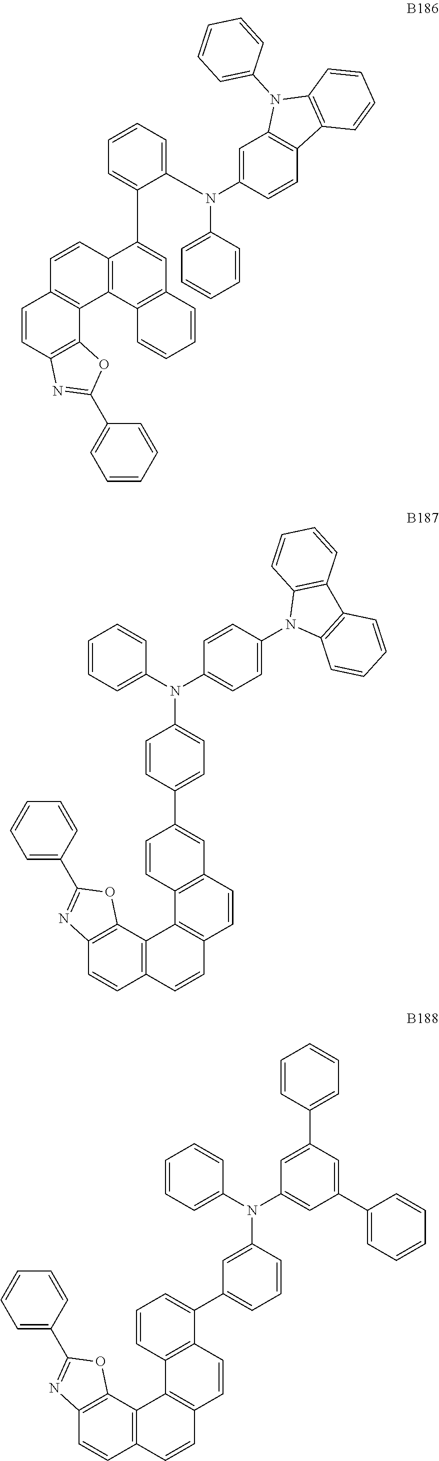 [SMILES: c1ccc(-c2nc3ccc4ccc5c(-c6ccccc6N(c6ccccc6)c6ccc7c8ccccc8n(-c8ccccc8)c7c6)cc6ccccc6c5c4c3o2)cc1 ; c1ccc(-c2nc3ccc4ccc5ccc6cc(-c7ccc(N(c8ccccc8)c8ccc(-n9c%10ccccc%10c%10ccccc%109)cc8)cc7)ccc6c5c4c3o2)cc1 ; c1ccc(-c2cc(-c3ccccc3)cc(N(c3ccccc3)c3cccc(-c4cccc5c4ccc4ccc6ccc7nc(-c8ccccc8)oc7c6c45)c3)c2)cc1]

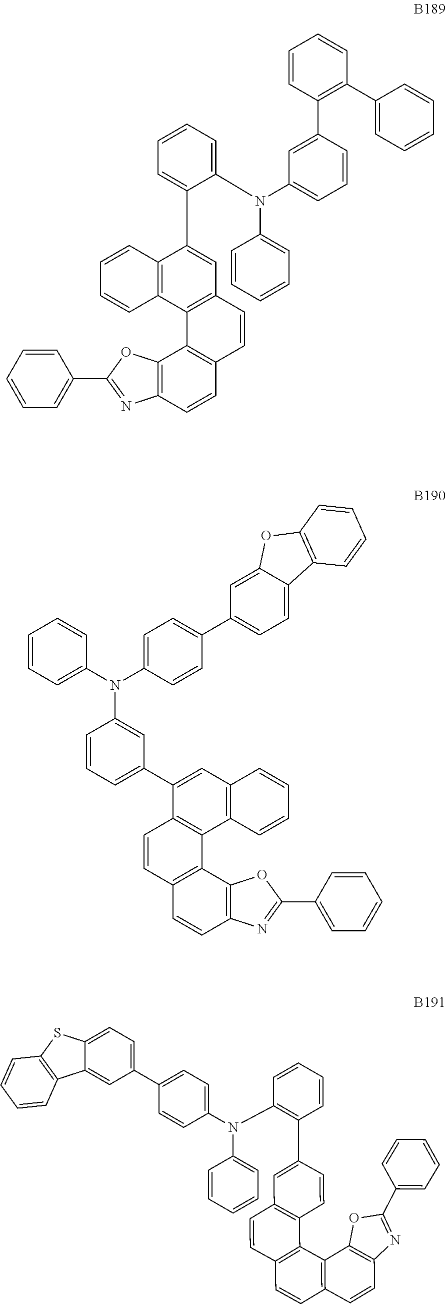 [SMILES: c1ccc(-c2nc3ccc4ccc5cc(-c6ccccc6N(c6ccccc6)c6cccc(-c7ccccc7-c7ccccc7)c6)c6ccccc6c5c4c3o2)cc1 ; c1ccc(-c2nc3ccc4ccc5c(-c6cccc(N(c7ccccc7)c7ccc(-c8ccc9c(c8)oc8ccccc89)cc7)c6)cc6ccccc6c5c4c3o2)cc1 ; c1ccc(-c2nc3ccc4ccc5ccc6cc(-c7ccccc7N(c7ccccc7)c7ccc(-c8ccc9sc%10ccccc%10c9c8)cc7)ccc6c5c4c3o2)cc1]

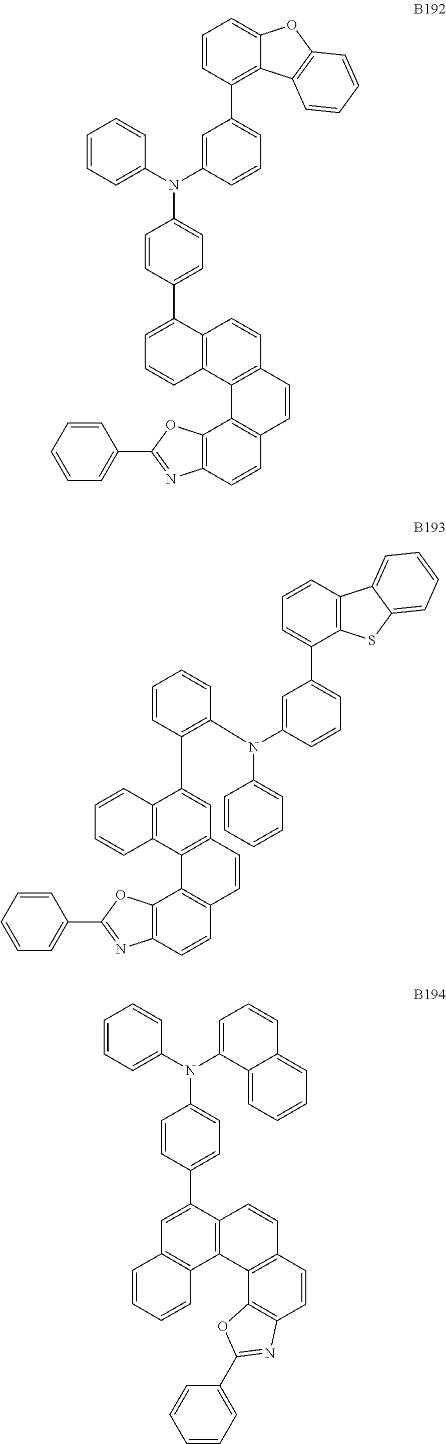 [SMILES: c1ccc(-c2nc3ccc4ccc5ccc6c(-c7ccc(N(c8ccccc8)c8cccc(-c9cccc%10oc%11ccccc%11c9%10)c8)cc7)cccc6c5c4c3o2)cc1 ; c1ccc(-c2nc3ccc4ccc5cc(-c6ccccc6N(c6ccccc6)c6cccc(-c7cccc8c7sc7ccccc78)c6)c6ccccc6c5c4c3o2)cc1 ; c1ccc(-c2nc3ccc4ccc5c(-c6ccc(N(c7ccccc7)c7cccc8ccccc78)cc6)cc6ccccc6c5c4c3o2)cc1]

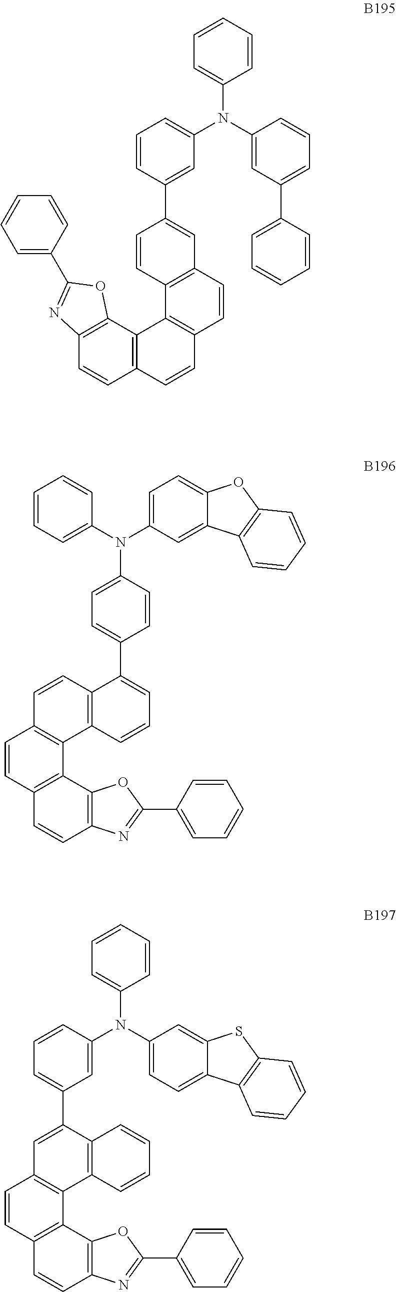 [SMILES: c1ccc(-c2cccc(N(c3ccccc3)c3cccc(-c4ccc5c(ccc6ccc7ccc8nc(-c9ccccc9)oc8c7c65)c4)c3)c2)cc1 ; c1ccc(-c2nc3ccc4ccc5ccc6c(-c7ccc(N(c8ccccc8)c8ccc9oc%10ccccc%10c9c8)cc7)cccc6c5c4c3o2)cc1 ; c1ccc(-c2nc3ccc4ccc5cc(-c6cccc(N(c7ccccc7)c7ccc8c(c7)sc7ccccc78)c6)c6ccccc6c5c4c3o2)cc1]

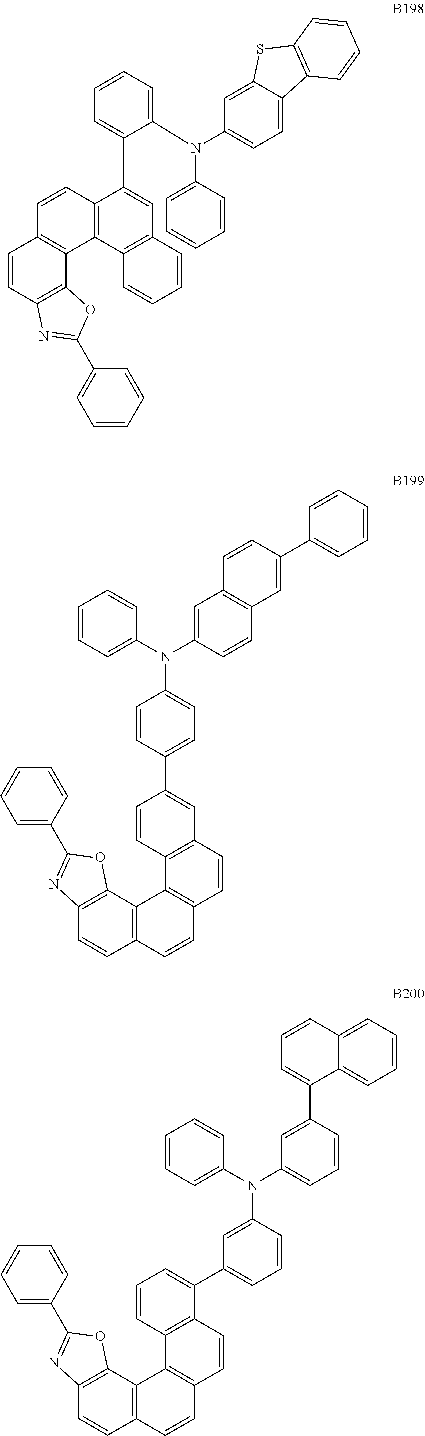 [SMILES: c1ccc(-c2nc3ccc4ccc5c(-c6ccccc6N(c6ccccc6)c6ccc7c(c6)sc6ccccc67)cc6ccccc6c5c4c3o2)cc1 ; c1ccc(-c2ccc3cc(N(c4ccccc4)c4ccc(-c5ccc6c(ccc7ccc8ccc9nc(-c%10ccccc%10)oc9c8c76)c5)cc4)ccc3c2)cc1 ; c1ccc(-c2nc3ccc4ccc5ccc6c(-c7cccc(N(c8ccccc8)c8cccc(-c9cccc%10ccccc9%10)c8)c7)cccc6c5c4c3o2)cc1]

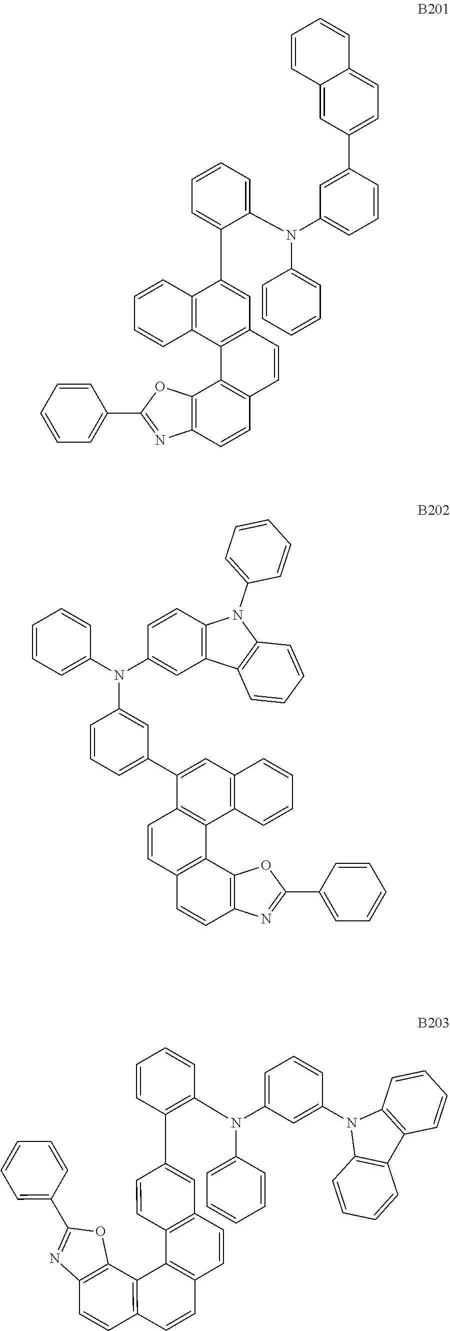 [SMILES: c1ccc(-c2nc3ccc4ccc5cc(-c6ccccc6N(c6ccccc6)c6cccc(-c7ccc8ccccc8c7)c6)c6ccccc6c5c4c3o2)cc1 ; c1ccc(-c2nc3ccc4ccc5c(-c6cccc(N(c7ccccc7)c7ccc8c(c7)c7ccccc7n8-c7ccccc7)c6)cc6ccccc6c5c4c3o2)cc1 ; c1ccc(-c2nc3ccc4ccc5ccc6cc(-c7ccccc7N(c7ccccc7)c7cccc(-n8c9ccccc9c9ccccc98)c7)ccc6c5c4c3o2)cc1]

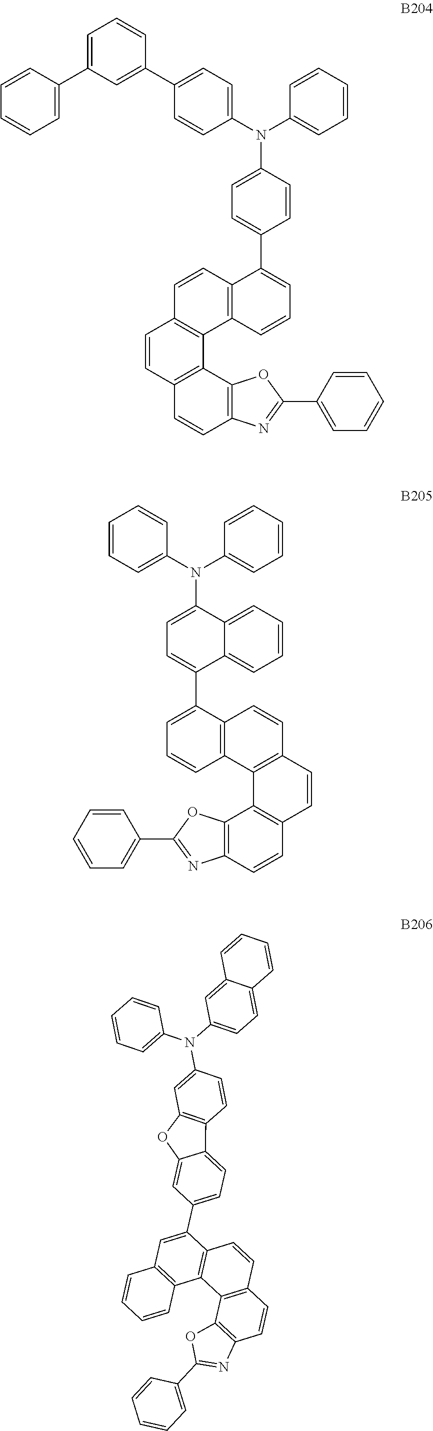 [SMILES: c1ccc(-c2cccc(-c3ccc(N(c4ccccc4)c4ccc(-c5cccc6c5ccc5ccc7ccc8nc(-c9ccccc9)oc8c7c56)cc4)cc3)c2)cc1 ; c1ccc(-c2nc3ccc4ccc5ccc6c(-c7ccc(N(c8ccccc8)c8ccccc8)c8ccccc78)cccc6c5c4c3o2)cc1 ; c1ccc(-c2nc3ccc4ccc5c(-c6ccc7c(c6)oc6cc(N(c8ccccc8)c8ccc9ccccc9c8)ccc67)cc6ccccc6c5c4c3o2)cc1]

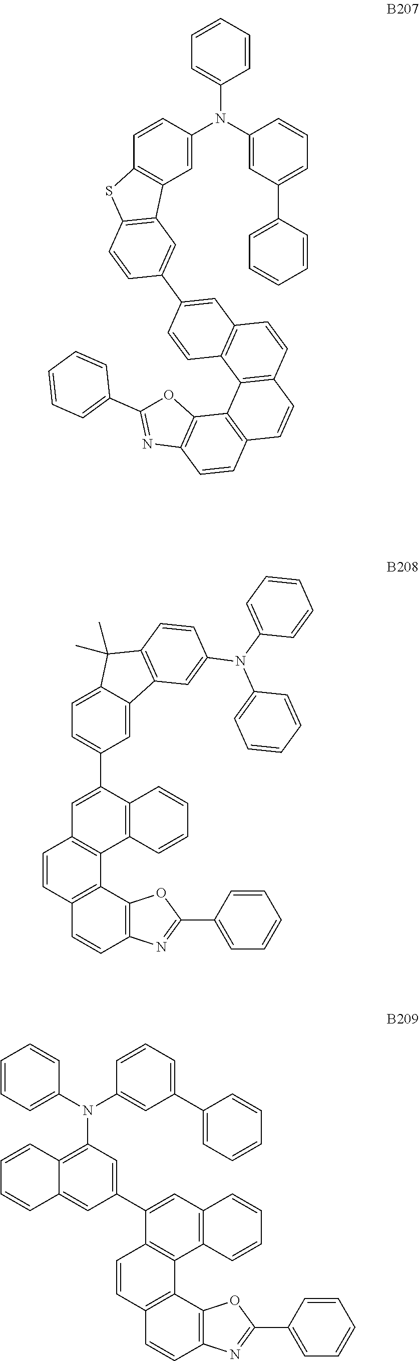 [SMILES: c1ccc(-c2cccc(N(c3ccccc3)c3ccc4sc5ccc(-c6ccc7c(ccc8ccc9ccc%10nc(-c%11ccccc%11)oc%10c9c87)c6)cc5c4c3)c2)cc1 ; CC1(C)c2ccc(-c3cc4ccc5ccc6nc(-c7ccccc7)oc6c5c4c4ccccc34)cc2-c2cc(N(c3ccccc3)c3ccccc3)ccc21 ; c1ccc(-c2cccc(N(c3ccccc3)c3cc(-c4cc5ccccc5c5c4ccc4ccc6nc(-c7ccccc7)oc6c45)cc4ccccc34)c2)cc1]

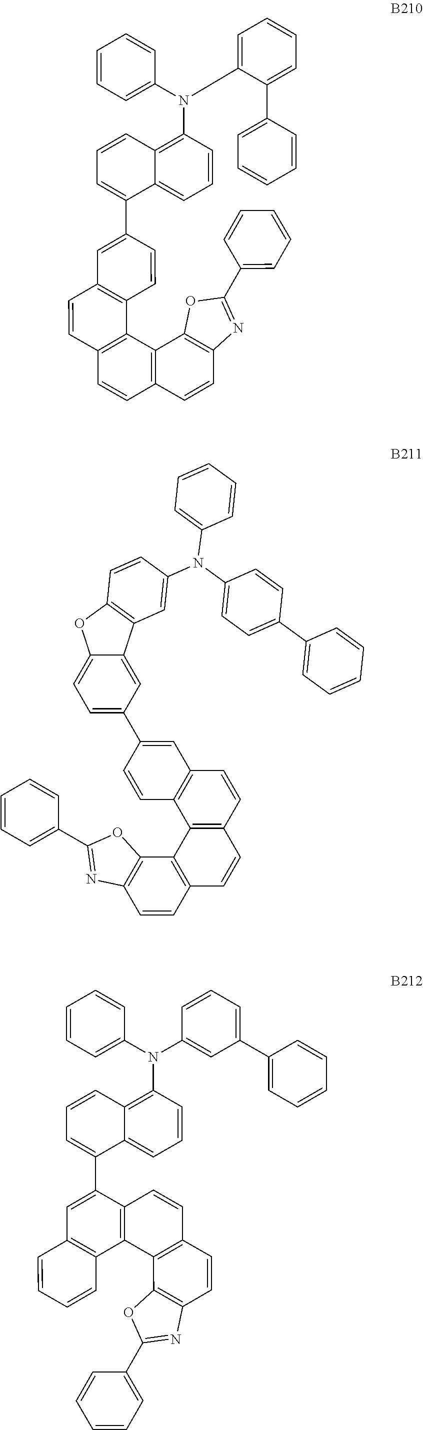 [SMILES: c1ccc(-c2nc3ccc4ccc5ccc6cc(-c7cccc8c(N(c9ccccc9)c9ccccc9-c9ccccc9)cccc78)ccc6c5c4c3o2)cc1 ; c1ccc(-c2ccc(N(c3ccccc3)c3ccc4oc5ccc(-c6ccc7c(ccc8ccc9ccc%10nc(-c%11ccccc%11)oc%10c9c87)c6)cc5c4c3)cc2)cc1 ; c1ccc(-c2cccc(N(c3ccccc3)c3cccc4c(-c5cc6ccccc6c6c5ccc5ccc7nc(-c8ccccc8)oc7c56)cccc34)c2)cc1]

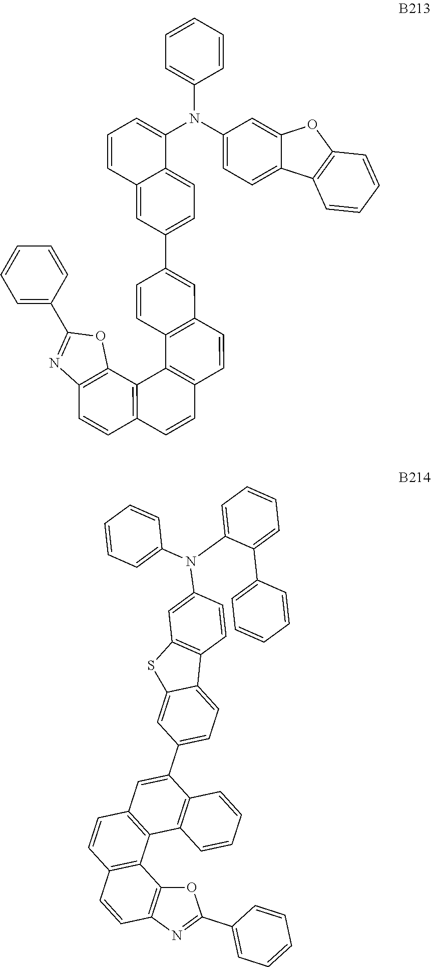 [SMILES: c1ccc(-c2nc3ccc4ccc5ccc6cc(-c7ccc8c(N(c9ccccc9)c9ccc%10c(c9)oc9ccccc9%10)cccc8c7)ccc6c5c4c3o2)cc1 ; c1ccc(-c2nc3ccc4ccc5cc(-c6ccc7c(c6)sc6cc(N(c8ccccc8)c8ccccc8-c8ccccc8)ccc67)c6ccccc6c5c4c3o2)cc1]

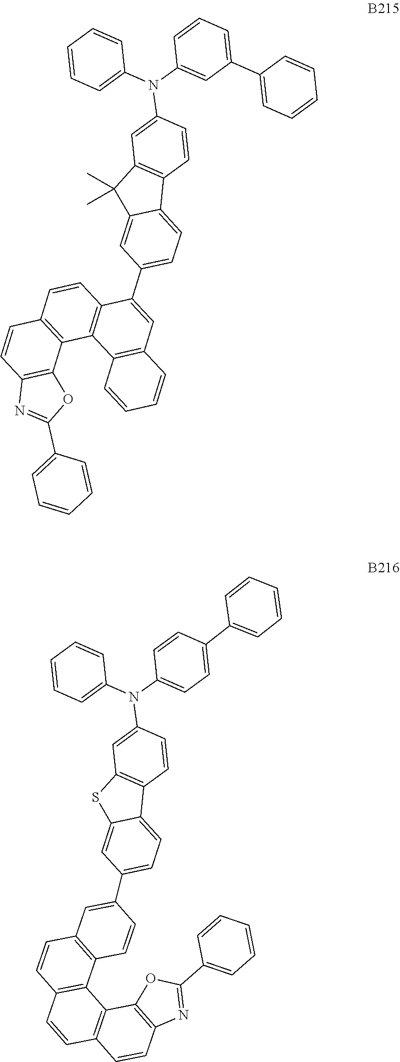 [SMILES: CC1(C)c2cc(-c3cc4ccccc4c4c3ccc3ccc5nc(-c6ccccc6)oc5c34)ccc2-c2ccc(N(c3ccccc3)c3cccc(-c4ccccc4)c3)cc21 ; c1ccc(-c2ccc(N(c3ccccc3)c3ccc4c(c3)sc3cc(-c5ccc6c(ccc7ccc8ccc9nc(-c%10ccccc%10)oc9c8c76)c5)ccc34)cc2)cc1]

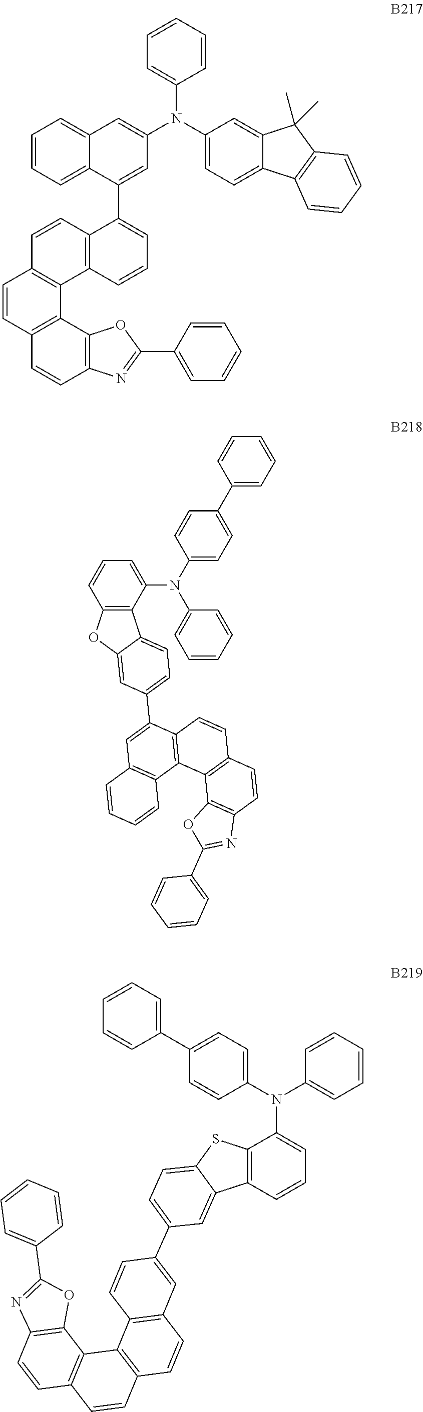 [SMILES: CC1(C)c2ccccc2-c2ccc(N(c3ccccc3)c3cc(-c4cccc5c4ccc4ccc6ccc7nc(-c8ccccc8)oc7c6c45)c4ccccc4c3)cc21 ; c1ccc(-c2ccc(N(c3ccccc3)c3cccc4oc5cc(-c6cc7ccccc7c7c6ccc6ccc8nc(-c9ccccc9)oc8c67)ccc5c34)cc2)cc1 ; c1ccc(-c2ccc(N(c3ccccc3)c3cccc4c3sc3ccc(-c5ccc6c(ccc7ccc8ccc9nc(-c%10ccccc%10)oc9c8c76)c5)cc34)cc2)cc1]

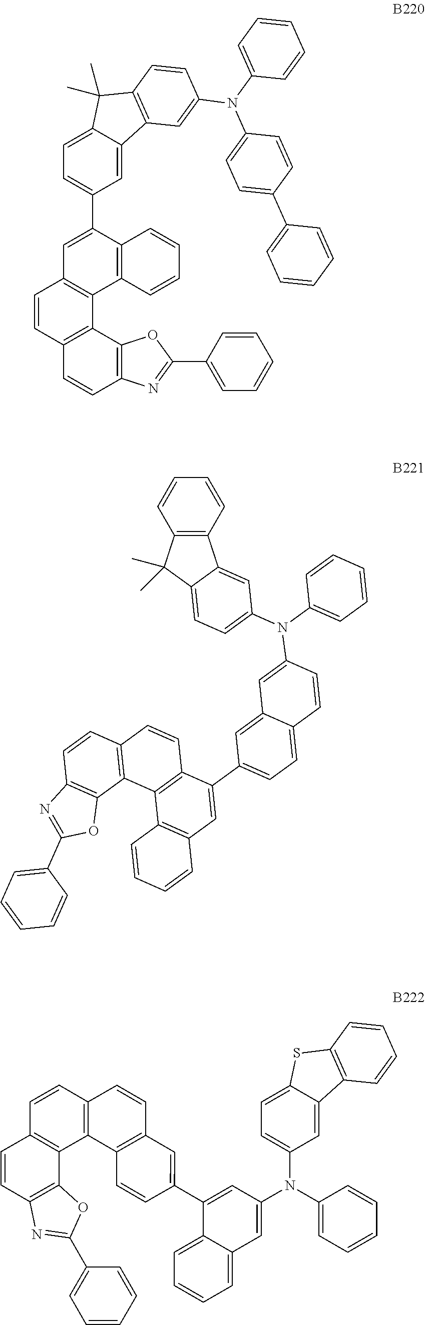 [SMILES: CC1(C)c2ccc(-c3cc4ccc5ccc6nc(-c7ccccc7)oc6c5c4c4ccccc34)cc2-c2cc(N(c3ccccc3)c3ccc(-c4ccccc4)cc3)ccc21 ; CC1(C)c2ccccc2-c2cc(N(c3ccccc3)c3ccc4ccc(-c5cc6ccccc6c6c5ccc5ccc7nc(-c8ccccc8)oc7c56)cc4c3)ccc21 ; c1ccc(-c2nc3ccc4ccc5ccc6cc(-c7cc(N(c8ccccc8)c8ccc9sc%10ccccc%10c9c8)cc8ccccc78)ccc6c5c4c3o2)cc1]

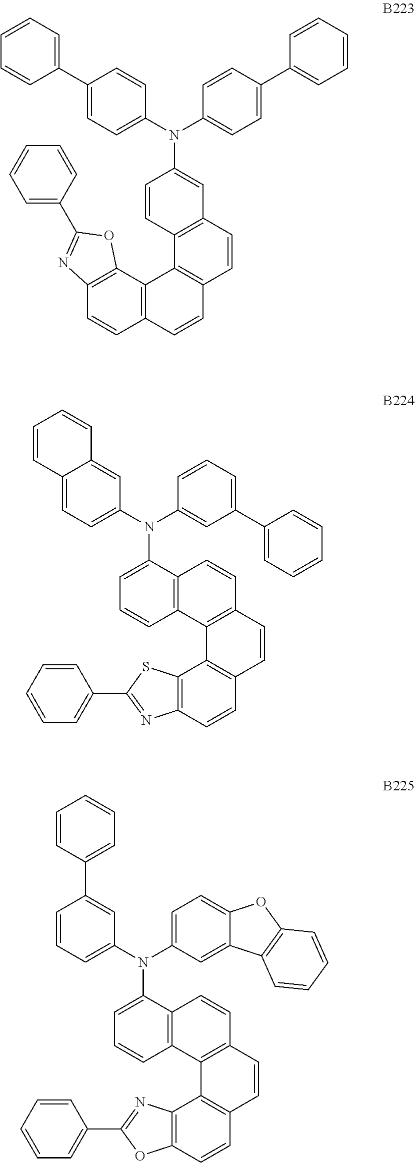 [SMILES: c1ccc(-c2ccc(N(c3ccc(-c4ccccc4)cc3)c3ccc4c(ccc5ccc6ccc7nc(-c8ccccc8)oc7c6c54)c3)cc2)cc1 ; c1ccc(-c2cccc(N(c3ccc4ccccc4c3)c3cccc4c3ccc3ccc5ccc6nc(-c7ccccc7)sc6c5c34)c2)cc1 ; c1ccc(-c2cccc(N(c3ccc4oc5ccccc5c4c3)c3cccc4c3ccc3ccc5ccc6oc(-c7ccccc7)nc6c5c34)c2)cc1]

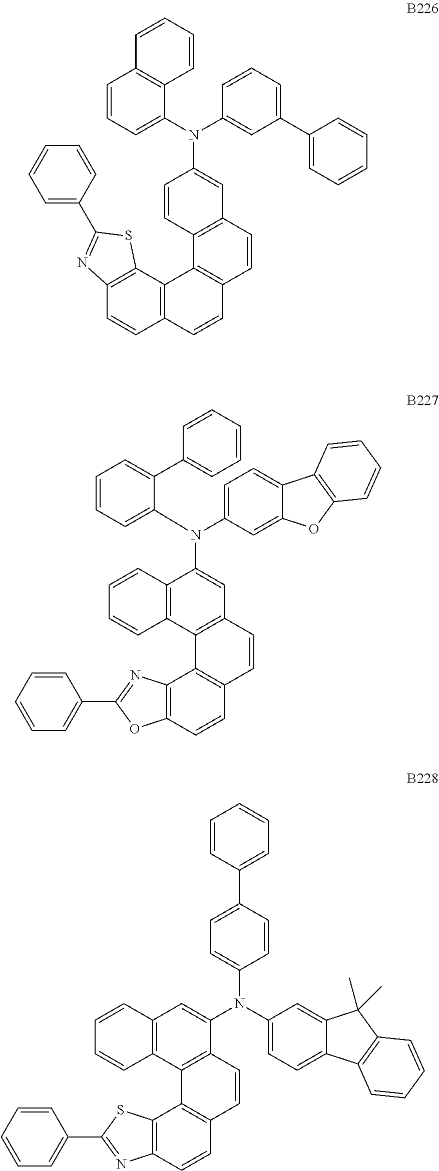 [SMILES: c1ccc(-c2cccc(N(c3ccc4c(ccc5ccc6ccc7nc(-c8ccccc8)sc7c6c54)c3)c3cccc4ccccc34)c2)cc1 ; c1ccc(-c2nc3c(ccc4ccc5cc(N(c6ccc7c(c6)oc6ccccc67)c6ccccc6-c6ccccc6)c6ccccc6c5c43)o2)cc1 ; CC1(C)c2ccccc2-c2ccc(N(c3ccc(-c4ccccc4)cc3)c3cc4ccccc4c4c3ccc3ccc5nc(-c6ccccc6)sc5c34)cc21]

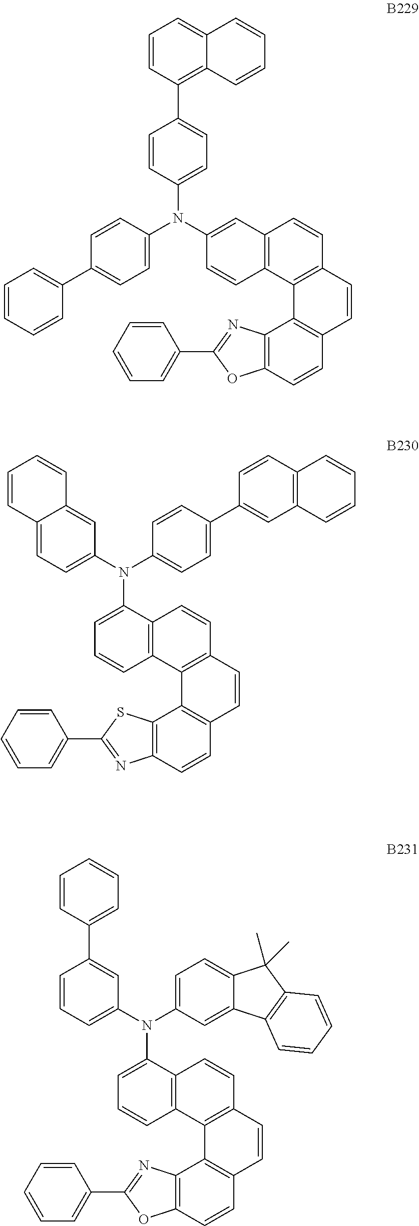 [SMILES: c1ccc(-c2ccc(N(c3ccc(-c4cccc5ccccc45)cc3)c3ccc4c(ccc5ccc6ccc7oc(-c8ccccc8)nc7c6c54)c3)cc2)cc1 ; c1ccc(-c2nc3ccc4ccc5ccc6c(N(c7ccc(-c8ccc9ccccc9c8)cc7)c7ccc8ccccc8c7)cccc6c5c4c3s2)cc1 ; CC1(C)c2ccccc2-c2cc(N(c3cccc(-c4ccccc4)c3)c3cccc4c3ccc3ccc5ccc6oc(-c7ccccc7)nc6c5c34)ccc21]

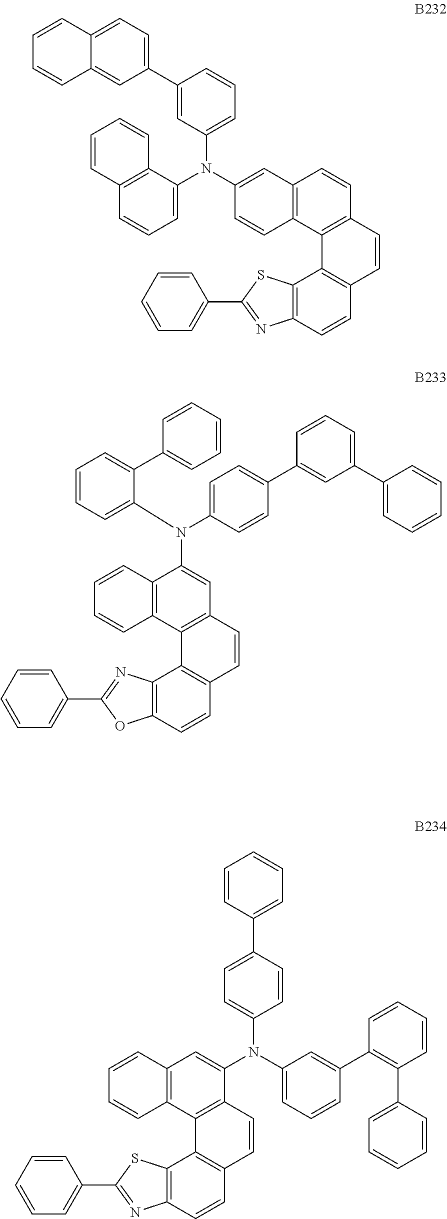 [SMILES: c1ccc(-c2nc3ccc4ccc5ccc6cc(N(c7cccc(-c8ccc9ccccc9c8)c7)c7cccc8ccccc78)ccc6c5c4c3s2)cc1 ; c1ccc(-c2cccc(-c3ccc(N(c4ccccc4-c4ccccc4)c4cc5ccc6ccc7oc(-c8ccccc8)nc7c6c5c5ccccc45)cc3)c2)cc1 ; c1ccc(-c2ccc(N(c3cccc(-c4ccccc4-c4ccccc4)c3)c3cc4ccccc4c4c3ccc3ccc5nc(-c6ccccc6)sc5c34)cc2)cc1]

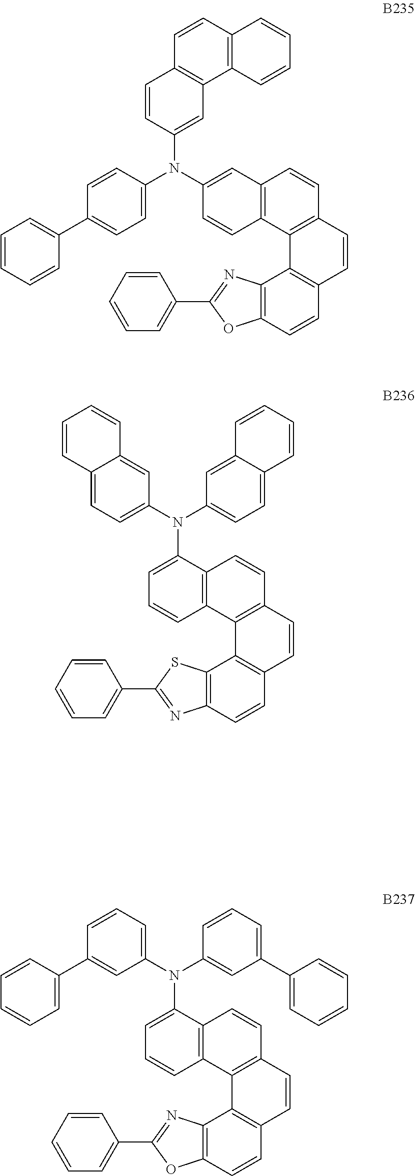 [SMILES: c1ccc(-c2ccc(N(c3ccc4c(ccc5ccc6ccc7oc(-c8ccccc8)nc7c6c54)c3)c3ccc4ccc5ccccc5c4c3)cc2)cc1 ; c1ccc(-c2nc3ccc4ccc5ccc6c(N(c7ccc8ccccc8c7)c7ccc8ccccc8c7)cccc6c5c4c3s2)cc1 ; c1ccc(-c2cccc(N(c3cccc(-c4ccccc4)c3)c3cccc4c3ccc3ccc5ccc6oc(-c7ccccc7)nc6c5c34)c2)cc1]

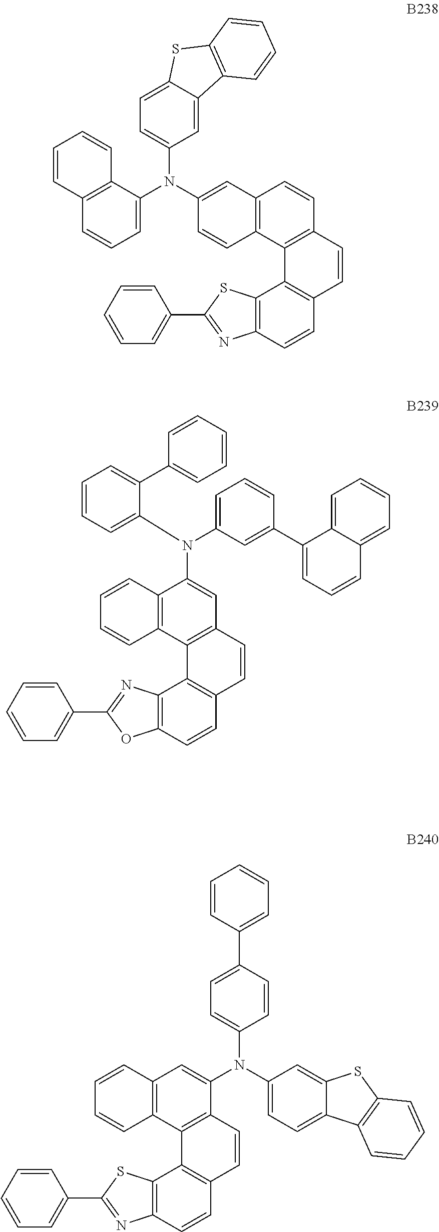 [SMILES: c1ccc(-c2nc3ccc4ccc5ccc6cc(N(c7ccc8sc9ccccc9c8c7)c7cccc8ccccc78)ccc6c5c4c3s2)cc1 ; c1ccc(-c2nc3c(ccc4ccc5cc(N(c6cccc(-c7cccc8ccccc78)c6)c6ccccc6-c6ccccc6)c6ccccc6c5c43)o2)cc1 ; c1ccc(-c2ccc(N(c3ccc4c(c3)sc3ccccc34)c3cc4ccccc4c4c3ccc3ccc5nc(-c6ccccc6)sc5c34)cc2)cc1]

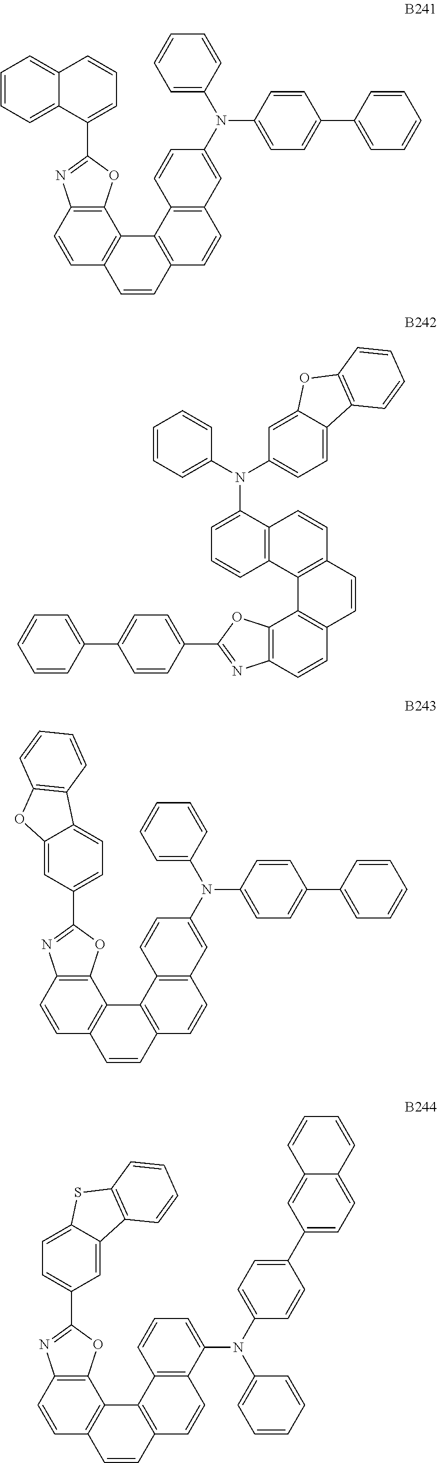 [SMILES: c1ccc(-c2ccc(N(c3ccccc3)c3ccc4c(ccc5ccc6ccc7nc(-c8cccc9ccccc89)oc7c6c54)c3)cc2)cc1 ; c1ccc(-c2ccc(-c3nc4ccc5ccc6ccc7c(N(c8ccccc8)c8ccc9c(c8)oc8ccccc89)cccc7c6c5c4o3)cc2)cc1 ; c1ccc(-c2ccc(N(c3ccccc3)c3ccc4c(ccc5ccc6ccc7nc(-c8ccc9c(c8)oc8ccccc89)oc7c6c54)c3)cc2)cc1 ; c1ccc(N(c2ccc(-c3ccc4ccccc4c3)cc2)c2cccc3c2ccc2ccc4ccc5nc(-c6ccc7sc8ccccc8c7c6)oc5c4c23)cc1]

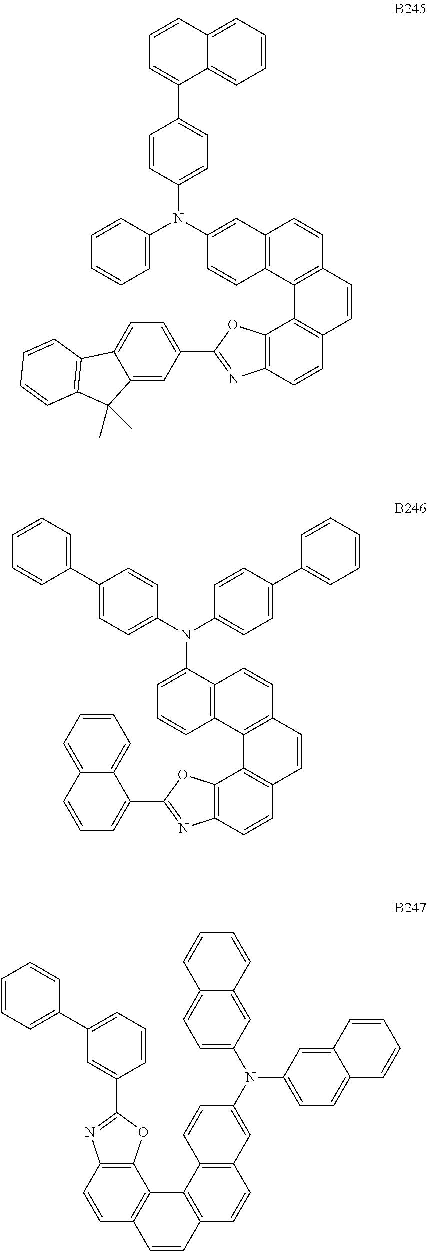 [SMILES: CC1(C)c2ccccc2-c2ccc(-c3nc4ccc5ccc6ccc7cc(N(c8ccccc8)c8ccc(-c9cccc%10ccccc9%10)cc8)ccc7c6c5c4o3)cc21 ; c1ccc(-c2ccc(N(c3ccc(-c4ccccc4)cc3)c3cccc4c3ccc3ccc5ccc6nc(-c7cccc8ccccc78)oc6c5c34)cc2)cc1 ; c1ccc(-c2cccc(-c3nc4ccc5ccc6ccc7cc(N(c8ccc9ccccc9c8)c8ccc9ccccc9c8)ccc7c6c5c4o3)c2)cc1]

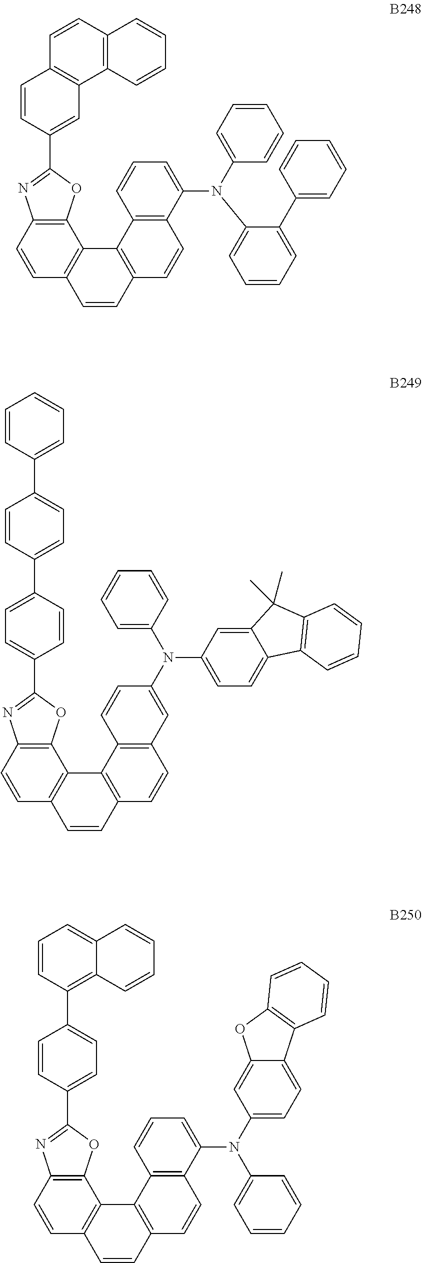 [SMILES: c1ccc(-c2ccccc2N(c2ccccc2)c2cccc3c2ccc2ccc4ccc5nc(-c6ccc7ccc8ccccc8c7c6)oc5c4c23)cc1 ; CC1(C)c2ccccc2-c2ccc(N(c3ccccc3)c3ccc4c(ccc5ccc6ccc7nc(-c8ccc(-c9ccc(-c%10ccccc%10)cc9)cc8)oc7c6c54)c3)cc21 ; c1ccc(N(c2ccc3c(c2)oc2ccccc23)c2cccc3c2ccc2ccc4ccc5nc(-c6ccc(-c7cccc8ccccc78)cc6)oc5c4c23)cc1]

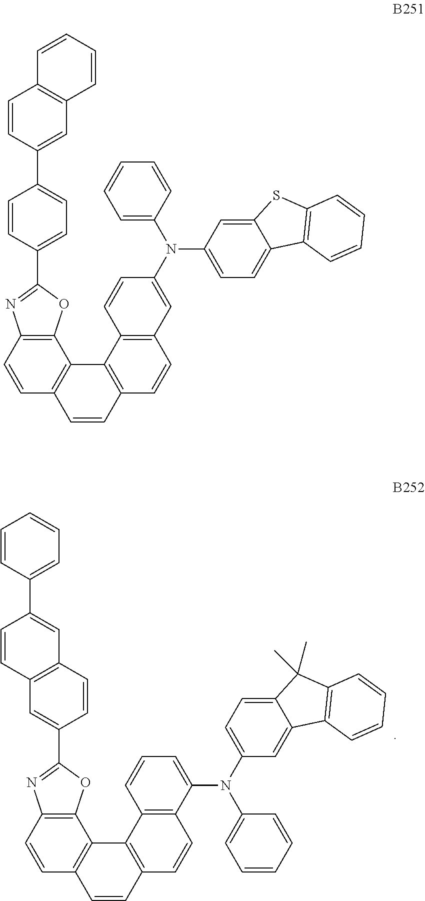 [SMILES: c1ccc(N(c2ccc3c(ccc4ccc5ccc6nc(-c7ccc(-c8ccc9ccccc9c8)cc7)oc6c5c43)c2)c2ccc3c(c2)sc2ccccc23)cc1 ; CC1(C)c2ccccc2-c2cc(N(c3ccccc3)c3cccc4c3ccc3ccc5ccc6nc(-c7ccc8cc(-c9ccccc9)ccc8c7)oc6c5c34)ccc21]

According to a second aspect of the present disclosure, an organic electroluminescent device is provided. The organic electroluminescent device comprises an anode and a cathode, and a functional layer disposed between the anode and the cathode, and the functional layer comprises the nitrogen-containing compound described in the first aspect of the present disclosure.

The nitrogen-containing compound provided in the present disclosure can be used to form at least one organic film layer in the functional layer, so as to improve characteristics such as luminescence efficiency and service life of the organic electroluminescent device.

Optionally, the functional layer comprises an organic light-emitting layer, and the organic light-emitting layer comprises the nitrogen-containing compound described above. The organic light-emitting layer may be composed of the nitrogen-containing compound provided in the present disclosure, or may be composed of the nitrogen-containing compound provided in the present disclosure together with other materials.

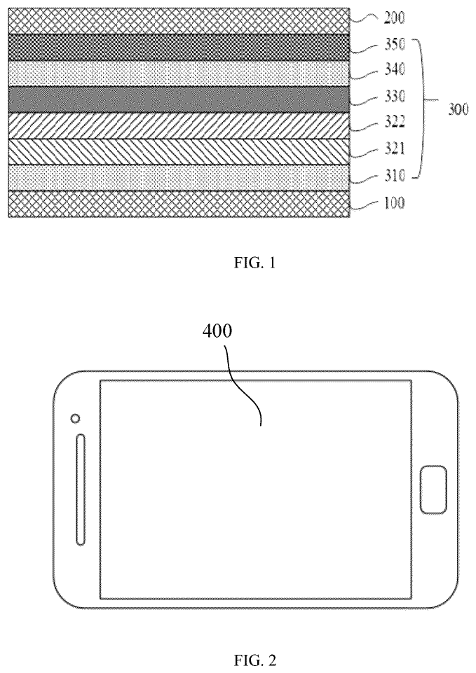

According to a specific embodiment, the organic electroluminescent device is as shown in FIG. 1. The organic electroluminescent device may comprise an anode 100, a hole injection layer 310, a first hole transport layer 321, a second hole transport layer (a hole auxiliary layer) 322, an organic light-emitting layer 330, an electron transport layer 340, an electron injection layer 350 and a cathode 200 that are stacked in sequence.

In the present disclosure, the anode 100 comprises the anode material, which is preferably a high-work function material contributing to injection of holes into the functional layer. Specific examples of the anode material include, but are not limited to: metals such as nickel, platinum, vanadium, chromium, copper, zinc and gold, or alloys thereof, metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly [3,4-(ethylene-1,2-dioxy)thiophene](PEDT), polypyrrole, and polyaniline. Preferably, a transparent electrode comprising indium tin oxide (ITO) is included as the anode.

In the present disclosure, the hole transport layer may include one or more hole transport materials. The hole transport materials may be selected from carbazole polymers, carbazole-linked triarylamine compounds, and other types of compounds, and specifically the hole transport layer is composed of the following compound or any combination thereof.

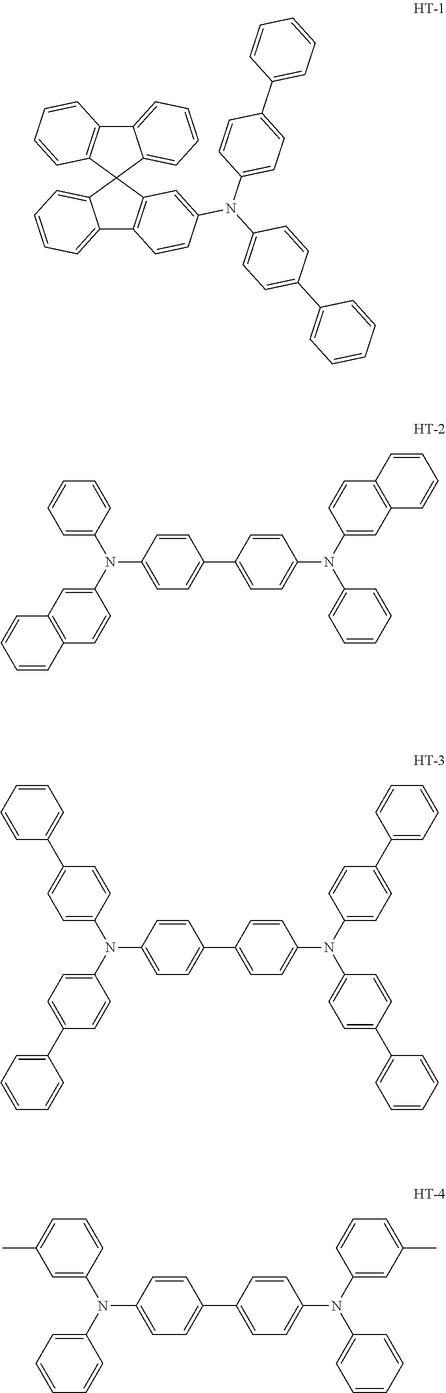

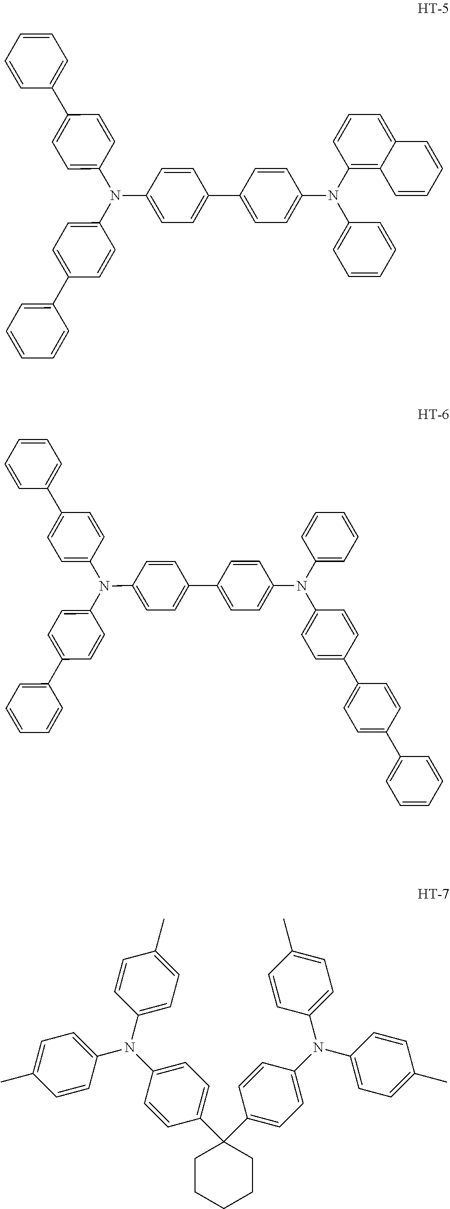

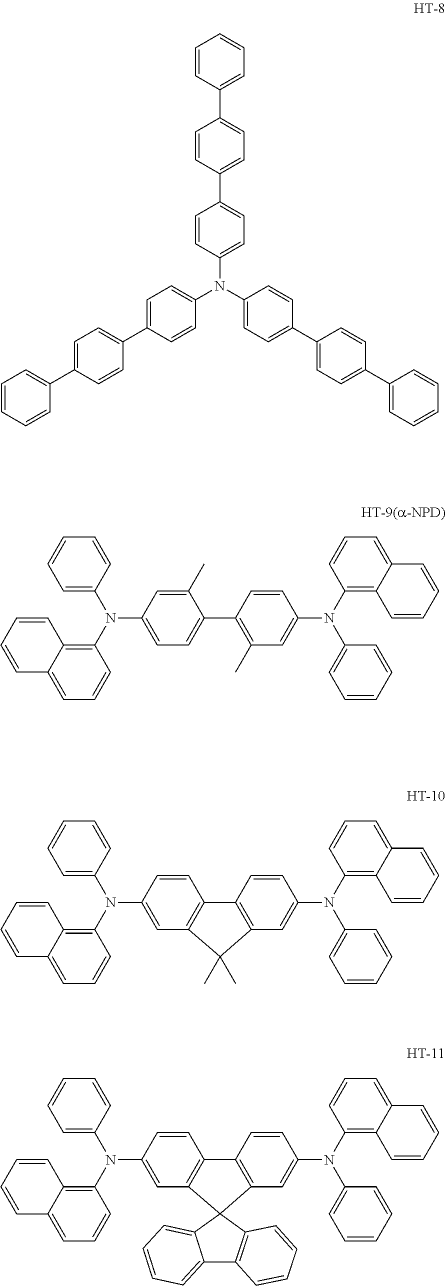 [SMILES: c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; Cc1cc(N(c2ccccc2)c2cccc3ccccc23)ccc1-c1ccc(N(c2ccccc2)c2cccc3ccccc23)cc1C ; CC1(C)c2cc(N(c3ccccc3)c3cccc4ccccc34)ccc2-c2ccc(N(c3ccccc3)c3cccc4ccccc34)cc21 ; c1ccc(N(c2ccc3c(c2)C2(c4ccccc4-c4ccccc42)c2cc(N(c4ccccc4)c4cccc5ccccc45)ccc2-3)c2cccc3ccccc23)cc1]

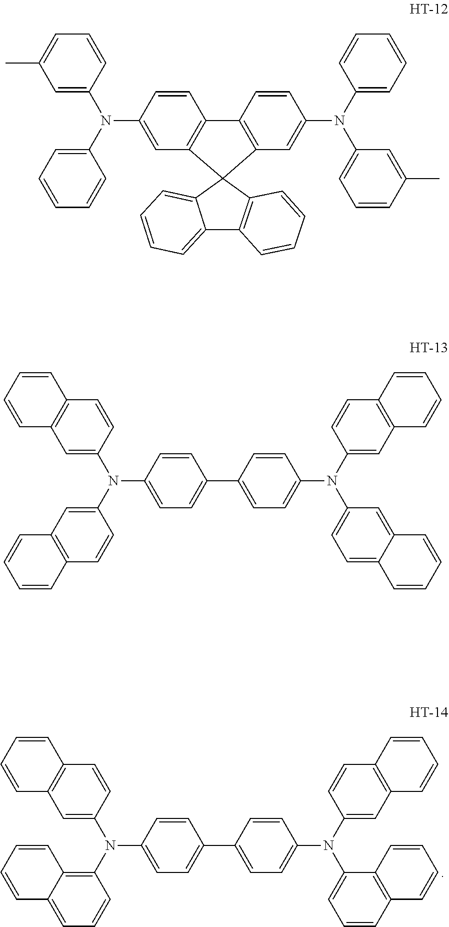

In an embodiment, the first hole transport layer 321 may be composed of α-NPD.

In an embodiment, the second hole transport layer 322 is composed of HT-1.

Optionally, a hole injection layer 310 may be further provided between the anode 100 and the first hole transport layer 321 to enhance the ability to inject holes into the first hole transport layer 321. The hole injection layer 310 may be selected from benzidine derivatives, starburst arylamine compounds, phthalocyanine derivatives or other materials, and the present disclosure is not particularly restricted in this respect. The material of the hole injection layer 310 is composed of the following compounds or any combination thereof:

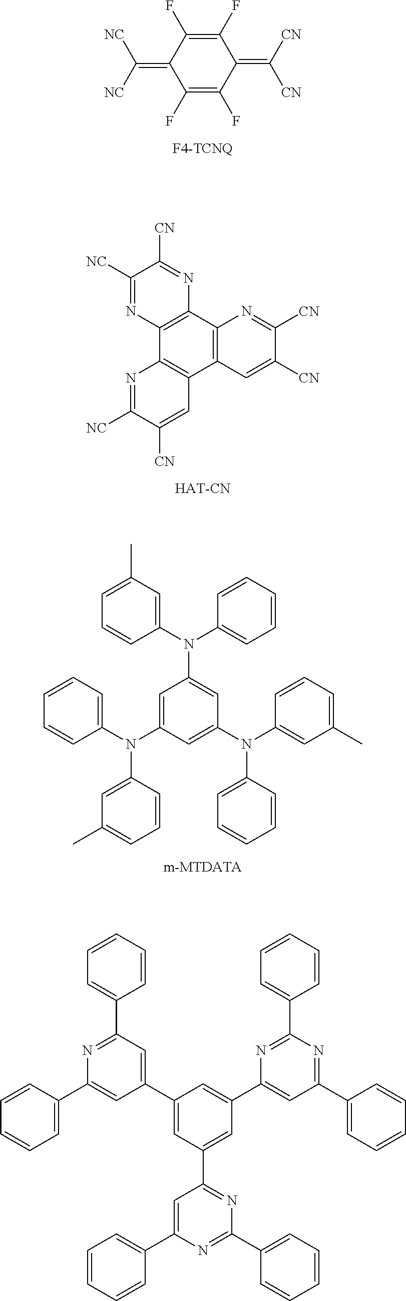 [SMILES: N#CC(C#N)=c1c(F)c(F)c(=C(C#N)C#N)c(F)c1F ; N#Cc1cc2c3cc(C#N)c(C#N)nc3c3nc(C#N)c(C#N)nc3c2nc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

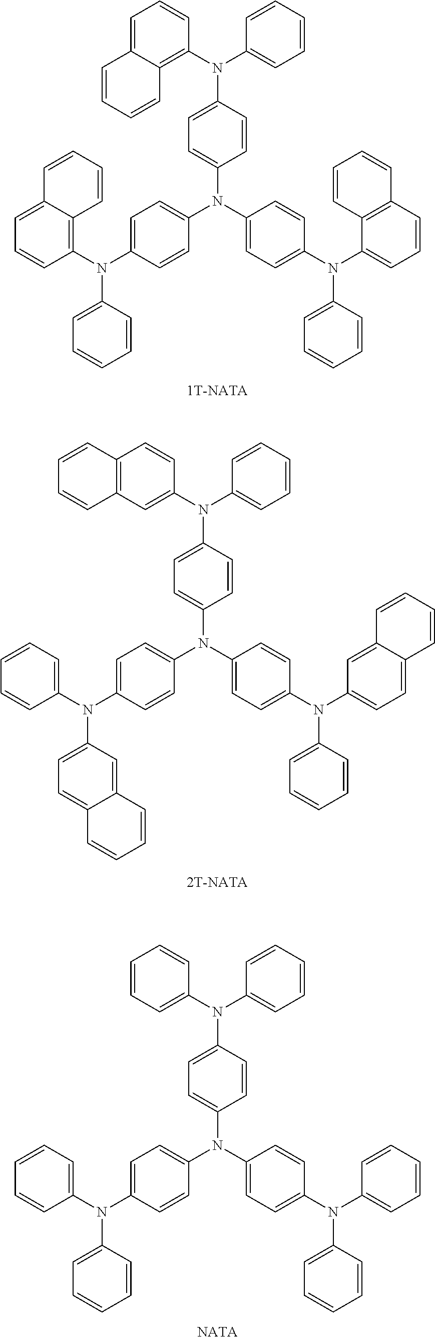

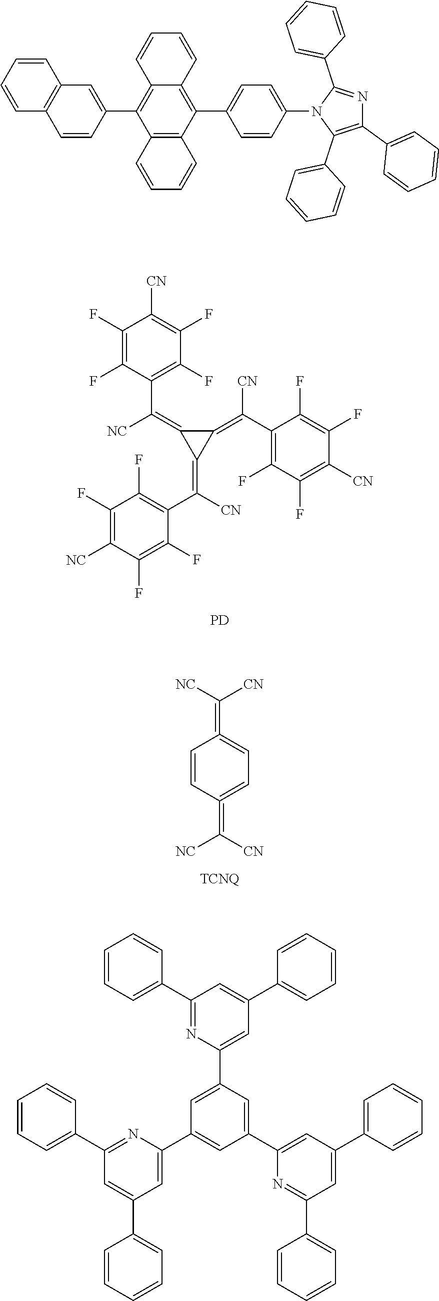

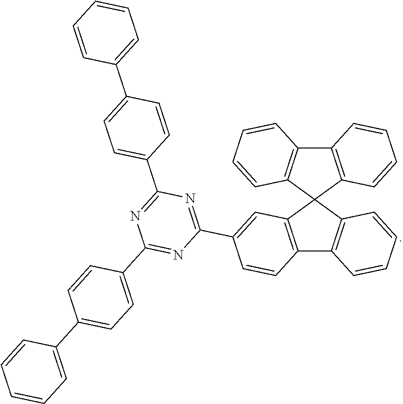 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In an embodiment, the hole injection layer 310 is composed of PD and α-NPD.

In the present disclosure, the organic light-emitting layer 330 may be composed of a single luminescent material, or may comprise a host material and a dopant material. Optionally, the organic light-emitting layer 330 is composed of a host material and a dopant material. Holes injected into the organic light-emitting layer 330 and electrons injected into the organic light-emitting layer 330 can recombine in the organic light-emitting layer 330 to form excitons. The excitons transmit energy to the host material, and the host material transmits the energy to the dopant material, thereby enabling the dopant material to emit light.

The host material of the organic light-emitting layer 330 may be a metal chelating compound, a stilbene derivative, an aromatic amine derivative, a dibenzofuran derivative, or other types of materials. Optionally, the host material includes the nitrogen-containing compound of the present disclosure.

The dopant material of the organic light-emitting layer 330 may be a compound having a condensed aryl ring or a derivative thereof, a compound having a heteroaryl ring or a derivative thereof, an aromatic amine derivative, or other materials, and the present disclosure is not particularly restricted in this respect. The dopant material is also known as a doped material or a dopant. According to the type of luminescence, it can be divided into a fluorescent dopant and a phosphorescent dopant. The specific examples of the phosphorescent dopant includes but is not limited to,

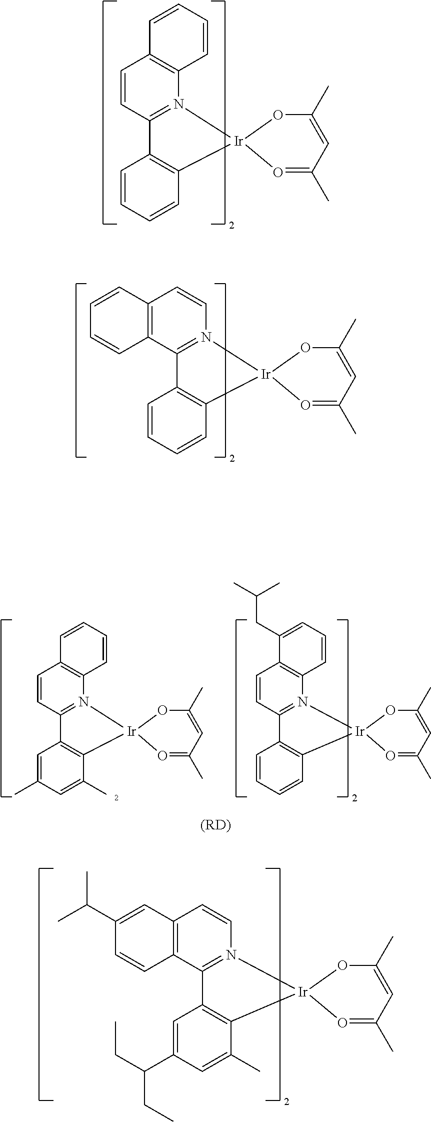

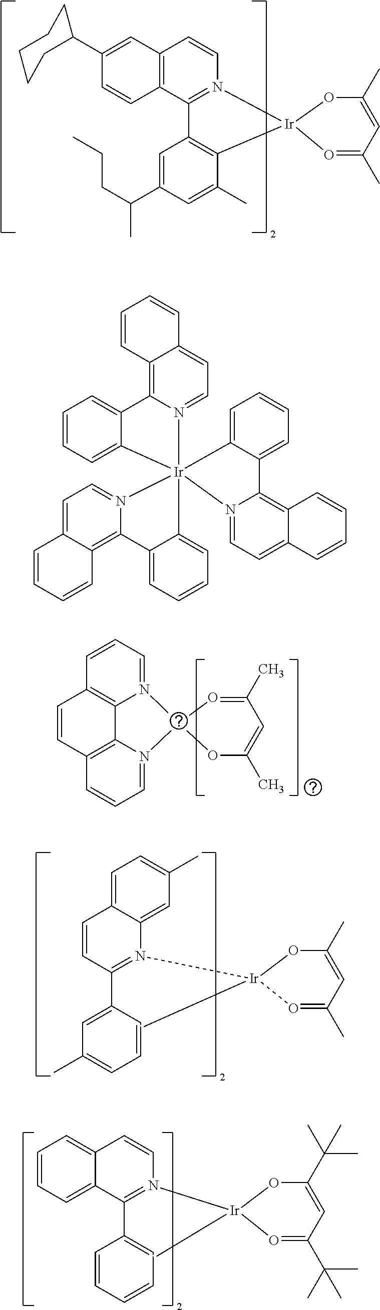

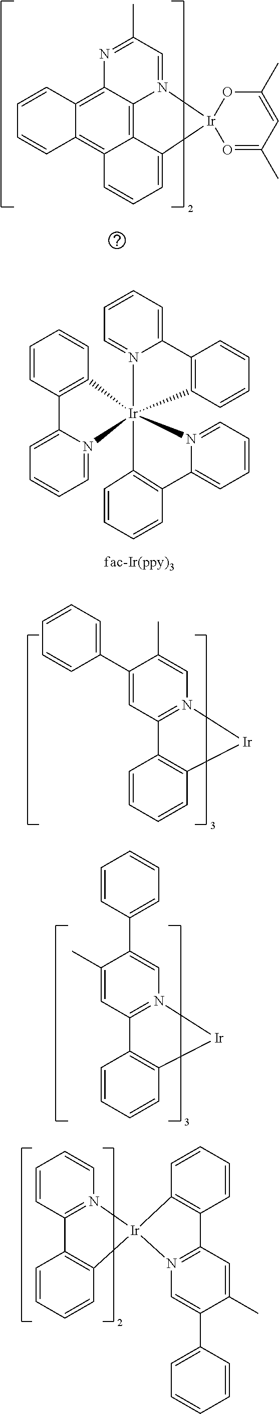

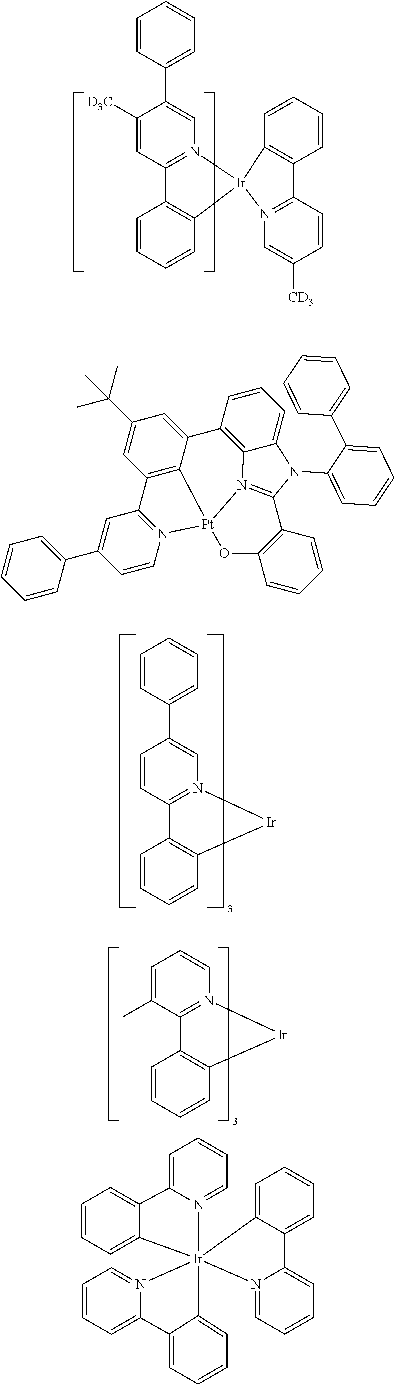

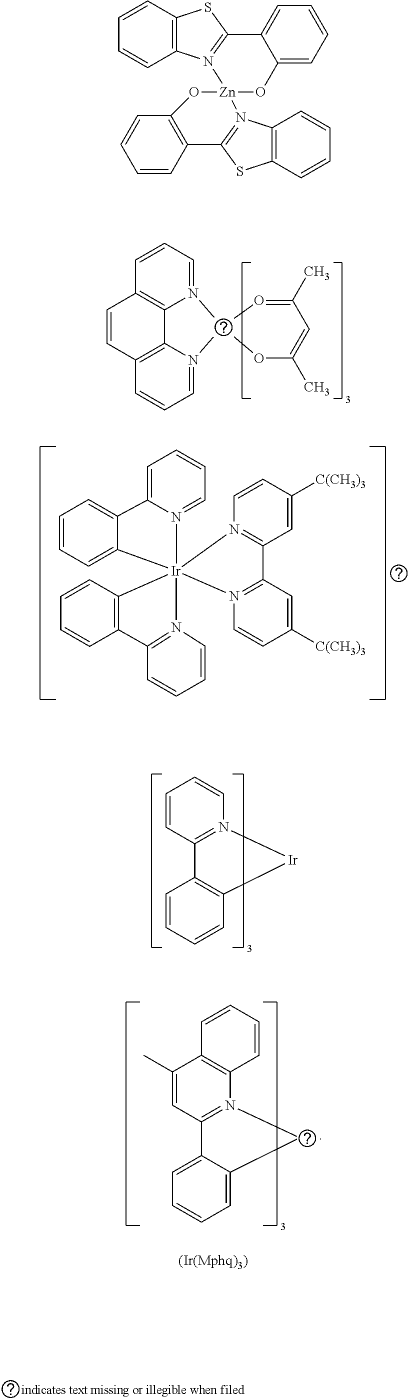

In an embodiment of the present disclosure, the organic electroluminescent device is a red electroluminescent device. In an embodiment, the host material of the organic light-emitting layer 330 comprises the nitrogen-containing compound of the present disclosure. The dopant material is, for example, RD.

In an embodiment, the host material of the organic light-emitting layer 330 comprises the nitrogen-containing compound of the present disclosure and

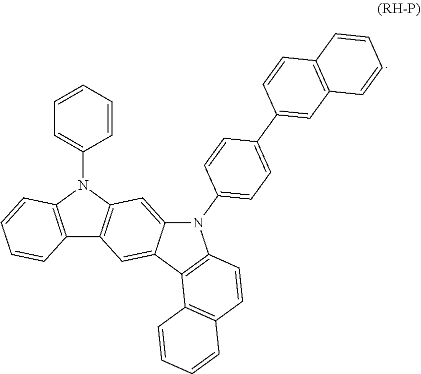 [SMILES: c1ccc(-n2c3ccccc3c3cc4c5c6ccccc6ccc5n(-c5ccc(-c6ccc7ccccc7c6)cc5)c4cc32)cc1]

In an embodiment, the host material of the organic light-emitting layer 330 includes the nitrogen-containing compound of the present disclosure and

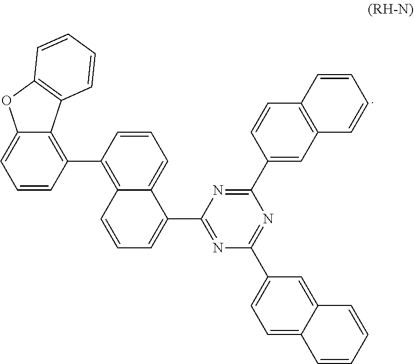 [SMILES: c1ccc2cc(-c3nc(-c4ccc5ccccc5c4)nc(-c4cccc5c(-c6cccc7oc8ccccc8c67)cccc45)n3)ccc2c1]

In an embodiment of the present disclosure, the organic electroluminescent device is a green electroluminescent device. In a more specific embodiment, the host material of the organic light-emitting layer 330 comprises the nitrogen-containing compound of the present disclosure.

The electron transport layer 340 may be a single-layer structure or a multi-layer structure, and may comprise one or more electron transport materials. The electron transport materials may be selected from BTB, LiQ, benzimidazole derivatives, oxadiazole derivatives, quinoxaline derivatives, and other electron transport materials, and the present disclosure is not particularly restricted in this respect. The material of the electron transport layer 340 includes but is not limit to the following compounds.

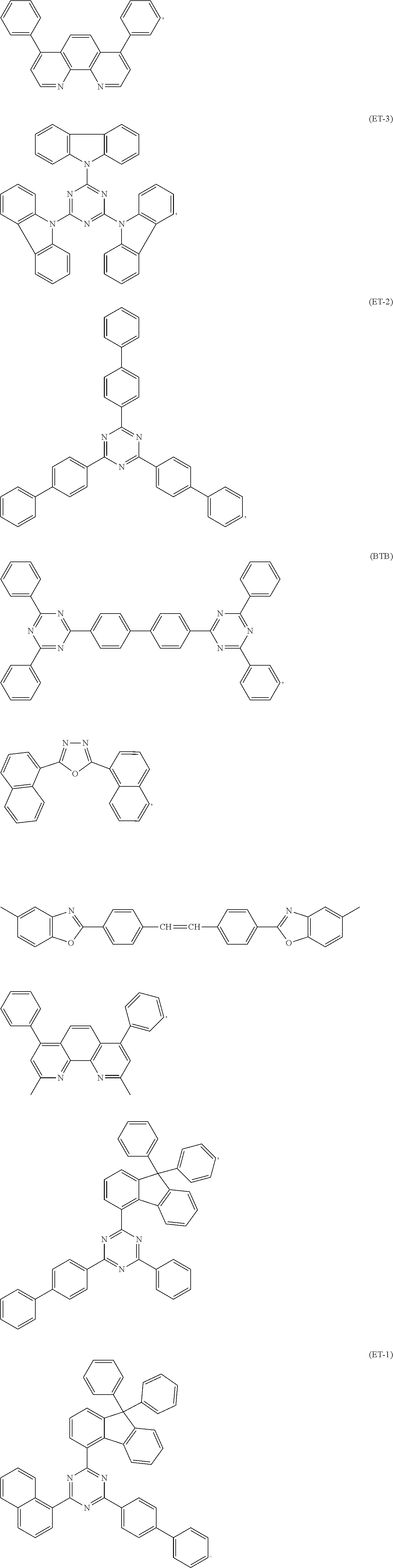

In an embodiment of the present disclosure, the electron transport layer 340 is composed of ET-1 and LiQ, or is composed of ET-2 and LiQ.

In an embodiment, the cathode 200 comprises a cathode material, which is a low-work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, lead, and alloys thereof; or multilayer materials such as LiF/Al, Liq/Al, LiO₂/Al, LiF/Ca, LiF/Al, and BaF₂/Ca. Optionally, a metal electrode comprising magnesium and silver is included as the cathode.

Optionally, an electron injection layer 350 may be further provided between the cathode 200 and the electron transport layer 340 to enhance the ability to inject electrons into the electron transport layer 340. The electron injection layer 350 may comprise an inorganic material such as an alkali metal sulfide, an alkali metal halide, or may comprise a complex of an alkali metal and an organic compound. In an embodiment of the present disclosure, the electron injection layer 350 may be composed of Yb.

According to a third aspect of the present disclosure, the electronic apparatus is provided, comprising the organic electroluminescent device described in the second aspect.

According to an embodiment, as shown in FIG. 2, the electronic apparatus is a electronic device 400 comprising the above described organic electroluminescent device. The electronic device 400 may be a display device, a lighting device, an optical communication device, or other type of electronic devices, examples of which may include, but are not limited to, computer screens, mobile phone screens, televisions, electronic paper, emergency lamps, optical modules, etc.

The synthesis of the nitrogen-containing compound of the present disclosure are described below in conjunction with synthesis examples, however, do not limit the present disclosure in any way.

### Synthesis Examples

Professionals in their field should realize that the chemical reactions described in this present disclosure can be used to properly prepare many of the organic compounds in this present disclosure, and other methods used to prepare the compounds in this present disclosure are considered to be within the scope of this present disclosure. For example, according to this present disclosure, the synthesis of those non-exemplified compounds can be successfully completed by the technicians in the field through modification methods, such as appropriate protection of interfering groups, by using other known reagents in addition to the ones described in this present disclosure, or by making some conventional modifications to the reaction conditions. The compounds of the synthetic methods not mentioned in this present disclosure are all raw material products obtained through commercial channels.

### Synthesis of Sub-a1:

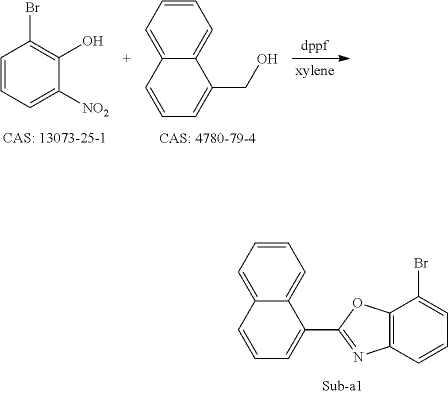 [SMILES: O=[N+]([O-])c1cccc(Br)c1O ; OCc1cccc2ccccc12 ; Brc1cccc2nc(-c3cccc4ccccc34)oc12]

Under a nitrogen atmosphere, 2-bromo-6-nitrophenol (10.9 g, 50 mmol), 1-naphthylmethanol (10.28 g, 65 mmol), 1,1′-bis(diphenylphosphine) ferrocene (0.83 g, 1.5 mmol) and xylene (100 mL) were added into a 250 mL three-necked flask in turn, and stirring and heating were initiated. The resulting mixture was heated to reflux and stirred to react for 48 hours. The reaction solution was cooled to room temperature, extracted with dichloromethane (100 mL×3 times), and the organic phases were combined and dried with anhydrous magnesium sulfate, followed by filtration and then reduced-pressure distillation to remove the solvent, obtaining a crude product. The crude product was purified by silica gel column chromatography with a mixture of dichloromethane and n-heptane as a mobile phase, to obtain an off-white solid Sub-a1 (7.29 g, yield 45%).

Referring to the synthesis of Sub-a1, Sub-a2 to Sub-a4 were synthesized by replacing 1-naphthylmethanol with Reactant A shown in Table 1.

TABLE 1
Synthesis of Sub-a2 to Sub-a4

| | | Structure of Sub- | Yield |
|---|---|---|---|
| Sub-aX number | Reactant A | aX | (%) |
| Sub-a2 | 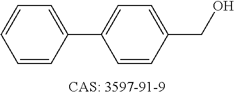 [SMILES: OCc1ccc(-c2ccccc2)cc1] | 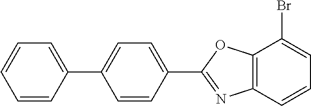 [SMILES: Brc1cccc2nc(-c3ccc(-c4ccccc4)cc3)oc12] | 48 |
| Sub-a3 | 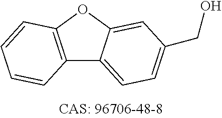 [SMILES: OCc1ccc2c(c1)oc1ccccc12] | 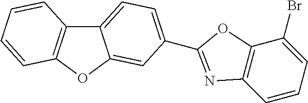 [SMILES: Brc1cccc2nc(-c3ccc4c(c3)oc3ccccc34)oc12] | 42 |
| Sub-a4 | 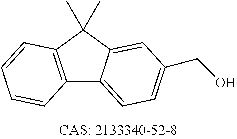 [SMILES: CC1(C)c2ccccc2-c2ccc(CO)cc21] | 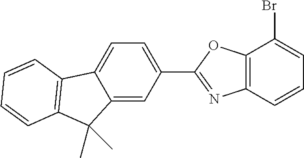 [SMILES: CC1(C)c2ccccc2-c2ccc(-c3nc4cccc(Br)c4o3)cc21] | 44 |

### Synthesis of Sub-b1:

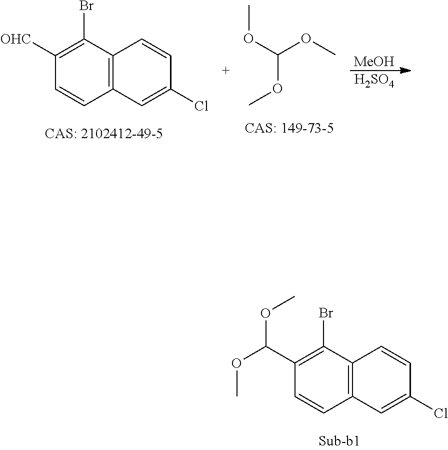

Under a nitrogen atmosphere, 1-bromo-6-chloro-2-naphthalenealdehyde (13.47 g, 50 mmol), trimethyl orthoformate (6.36 g, 60 mmol), methanol (150 mL) and a drop of concentrated sulfuric acid were added into a 500 mL three-necked flask in turn, and then stirring and heating were initiated. The resulting mixture was heated to reflux to react for 2 hours. After being cooled to room temperature, the reaction solution was neutralized with sodium methoxide followed by reduced-pressure distillation to remove the solvent, obtaining a crude product. The crude product was purified by silica gel column chromatography with n-heptane as a mobile phase, to obtain a colorless oily liquid Sub-b1 (14.52 g, yield 92%).

Referring to the synthesis of Sub-b1, Sub-b2 to Sub-b5 were synthesized by replacing 1-bromo-6-chloro-2-naphthalenealdehyde with Reactant B shown in Table 2.

TABLE 2
Synthesis of Sub-b2 to Sub-b5

| Sub-bX | | Structure of | Yield |
|---|---|---|---|
| number | Reactant B | Sub-bX | (%) |
| Sub-b2 | 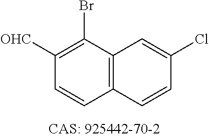 [SMILES: O=Cc1ccc2ccc(Cl)cc2c1Br] | 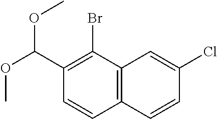 [SMILES: COC(OC)c1ccc2ccc(Cl)cc2c1Br] | 96 |
| Sub-b3 | 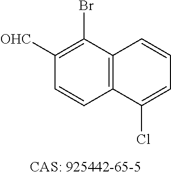 [SMILES: O=Cc1ccc2c(Cl)cccc2c1Br] | 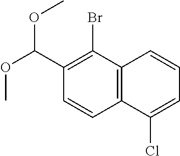 [SMILES: COC(OC)c1ccc2c(Cl)cccc2c1Br] | 92 |
| Sub-b4 | 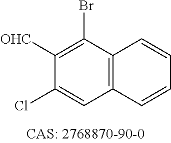 [SMILES: O=Cc1c(Cl)cc2ccccc2c1Br] | 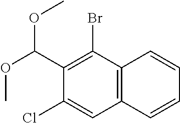 [SMILES: COC(OC)c1c(Cl)cc2ccccc2c1Br] | 84 |
| Sub-b5 | 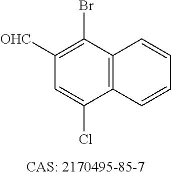 [SMILES: O=Cc1cc(Cl)c2ccccc2c1Br] | 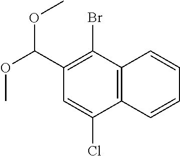 [SMILES: COC(OC)c1cc(Cl)c2ccccc2c1Br] | 88 |

### Synthesis of Sub-c1:

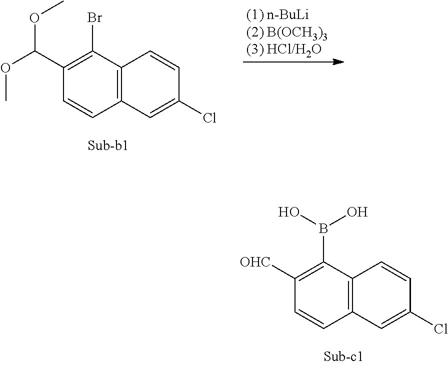 [SMILES: COC(OC)c1ccc2cc(Cl)ccc2c1Br ; O=Cc1ccc2cc(Cl)ccc2c1B(O)O]

Under a nitrogen atmosphere, to a 500 mL three-necked flask was added Sub-b1 (22.1 g, 70 mmol) and tetrahydrofuran (220 mL) and the resulting mixture was cooled to −78° C., followed by the dropwise addition of n-butyl lithium solution (2.0 M in n-hexane, 38.5 mL, 77 mmol). After the dropwise addition was completed, the resulting mixture was kept at −78° C. and stirred for 1 hour. Trimethyl borate (10.91 g, 105 mmol) was added dropwise at −78° C.; after the addition of trimethyl borate was completed, the reaction solution was kept at −78° C. for 1 hour, and then naturally wormed to room temperature. To the reaction solution was added dilute hydrochloric acid (2M, 58 mL) dropwise and stirred for 30 minutes. Then the reaction solution was extracted with dichloromethane (100 mL×3 times), the organic phases were combined and dried with anhydrous magnesium sulfate, followed by filtration and then reduced-pressure distillation to remove the solvent, obtaining an oily crude product. To the crude product was added 25 mL deionized water and three drops of concentrated hydrochloric acid, and the resulting solution was heated to 70° C. and stirred for 15 minutes. After the system was cooled to room temperature, it was extracted with dichloromethane (25 mL×3 times). The organic phases were combined and dried with anhydrous magnesium sulfate followed by filtration and then reduced-pressure distillation to remove the solvent, obtaining a solid crude product. The crude product was beaten with n-heptane and filtered to obtain a white solid product Sub-c1 (10.17 g, yield 62%).

Referring to the synthesis of Sub-c1, Sub-c2 to Sub-c5 were synthesized by replacing Sub-b1 with Reactant C shown in Table 3.

TABLE 3
Synthesis of Sub-c2 to Sub-c5

| | | Structure of | Yield |
|---|---|---|---|
| Sub-cX number | Reactant C | Sub-cX | (%) |
| Sub-c2 | 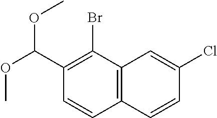 [SMILES: COC(OC)c1ccc2ccc(Cl)cc2c1Br] | 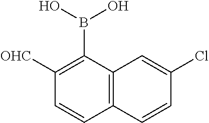 [SMILES: O=Cc1ccc2ccc(Cl)cc2c1B(O)O] | 66 |
| Sub-c3 | 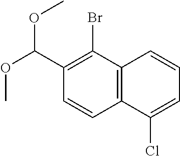 [SMILES: COC(OC)c1ccc2c(Cl)cccc2c1Br] | 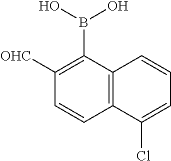 [SMILES: O=Cc1ccc2c(Cl)cccc2c1B(O)O] | 58 |
| Sub-c4 | 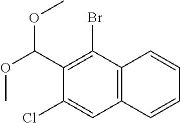 [SMILES: COC(OC)c1c(Cl)cc2ccccc2c1Br] | 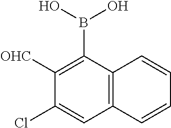 [SMILES: O=Cc1c(Cl)cc2ccccc2c1B(O)O] | 60 |
| Sub-c5 | 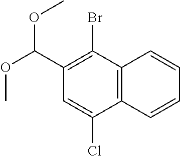 [SMILES: COC(OC)c1cc(Cl)c2ccccc2c1Br] | 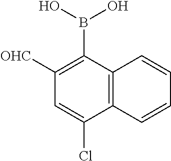 [SMILES: O=Cc1cc(Cl)c2ccccc2c1B(O)O] | 61 |

### Synthesis of Sub-d1:

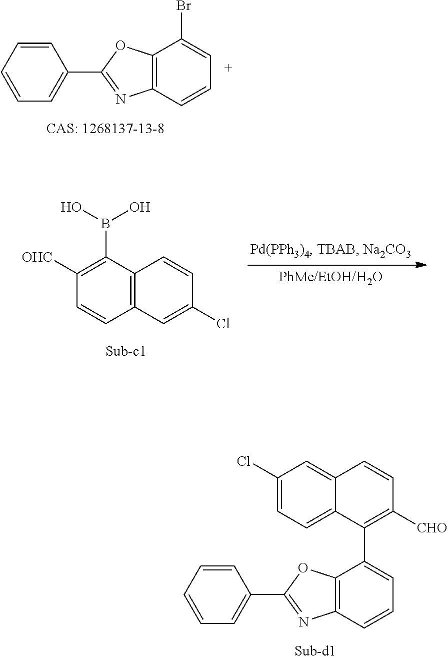 [SMILES: Brc1cccc2nc(-c3ccccc3)oc12 ; O=Cc1ccc2cc(Cl)ccc2c1B(O)O ; O=Cc1ccc2cc(Cl)ccc2c1-c1cccc2nc(-c3ccccc3)oc12]

Under a nitrogen atmosphere, 7-bromo-2-phenylbenzoxazole (13.71 g, 50 mmol), Sub-c1 (12.9 g, 55 mmol), tetrakis (triphenylphosphine) palladium (0.58 g, 0.5 mmol), anhydrous sodium carbonate (10.60 g, 100 mmol), toluene (140 mL), anhydrous ethanol (35 mL) and deionized water (35 mL) were added to a 500 mL three-necked flask sequentially, and then stirred and heated, the resulting mixture was heated up to reflux to react for 8 hours. After the reaction solution was cooled to room temperature, it was extracted with dichloromethane (100 mL×3 times), and the organic phases were combined and dried with anhydrous magnesium sulfate, followed by filtration and then reduced-pressure distillation, to remove the solvent, obtaining a crude product. The crude product was purified by silica gel column chromatography with a mixture of dichloromethane and n-heptane as a mobile phase, obtaining an orange-yellow solid Sub-d1 (14.78 g, yield 77%).

Referring to the synthesis of Sub-d1, Sub-d2 to Sub-d12 were synthesized by replacing 7-bromo-2-phenylbenzoxazole with Reactant D, and replacing Sub-c1 with Reactant E as shown in Table 4.

TABLE 4
Synthesis of Sub-d2 to Sub-d12

| Sub- | | | | |
|---|---|---|---|---|
| dX | | | | |
| num- | | | Structure of Sub- | Yield |
| ber | Reactant D | Reactant E | dX | (%) |
| Sub- d2 | 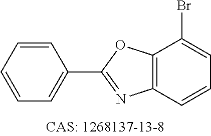 [SMILES: Brc1cccc2nc(-c3ccccc3)oc12] | 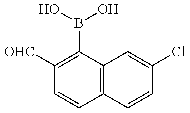 [SMILES: O=Cc1ccc2ccc(Cl)cc2c1B(O)O] | 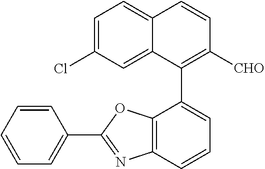 [SMILES: O=Cc1ccc2ccc(Cl)cc2c1-c1cccc2nc(-c3ccccc3)oc12] | 76 |
| Sub- d3 | 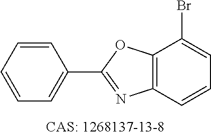 [SMILES: Brc1cccc2nc(-c3ccccc3)oc12] | 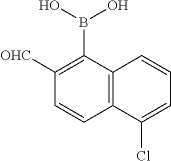 [SMILES: O=Cc1ccc2c(Cl)cccc2c1B(O)O] | 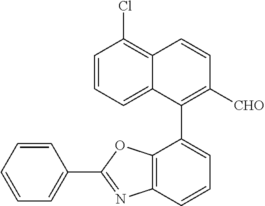 [SMILES: O=Cc1ccc2c(Cl)cccc2c1-c1cccc2nc(-c3ccccc3)oc12] | 77 |
| Sub- d4 | 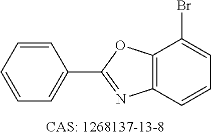 [SMILES: Brc1cccc2nc(-c3ccccc3)oc12] | 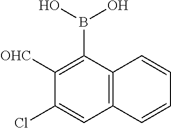 [SMILES: O=Cc1c(Cl)cc2ccccc2c1B(O)O] | 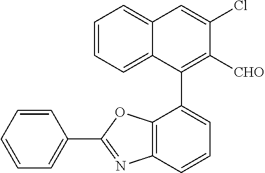 [SMILES: O=Cc1c(Cl)cc2ccccc2c1-c1cccc2nc(-c3ccccc3)oc12] | 73 |
| Sub- d5 | 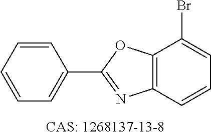 [SMILES: Brc1cccc2nc(-c3ccccc3)oc12] | 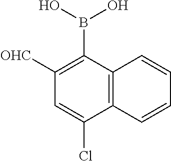 [SMILES: O=Cc1cc(Cl)c2ccccc2c1B(O)O] | 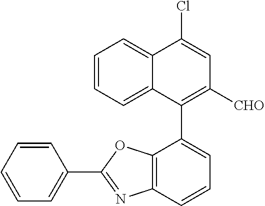 [SMILES: O=Cc1cc(Cl)c2ccccc2c1-c1cccc2nc(-c3ccccc3)oc12] | 70 |
| Sub- d6 | 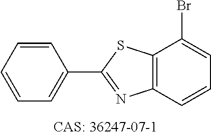 [SMILES: Brc1cccc2nc(-c3ccccc3)sc12] | 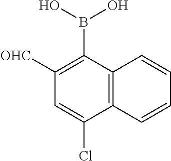 [SMILES: O=Cc1cc(Cl)c2ccccc2c1B(O)O] | 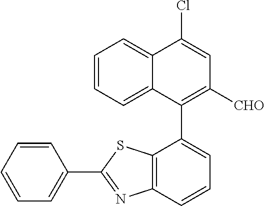 [SMILES: O=Cc1cc(Cl)c2ccccc2c1-c1cccc2nc(-c3ccccc3)sc12] | 80 |
| Sub- d7 | 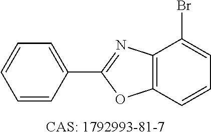 [SMILES: Brc1cccc2oc(-c3ccccc3)nc12] | 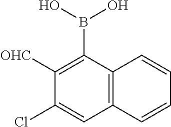 [SMILES: O=Cc1c(Cl)cc2ccccc2c1B(O)O] | 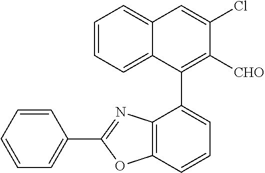 [SMILES: O=Cc1c(Cl)cc2ccccc2c1-c1cccc2oc(-c3ccccc3)nc12] | 78 |
| Sub- d8 | 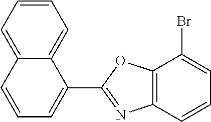 [SMILES: Brc1cccc2nc(-c3cccc4ccccc34)oc12] | 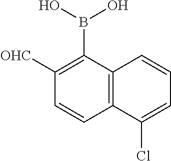 [SMILES: O=Cc1ccc2c(Cl)cccc2c1B(O)O] | 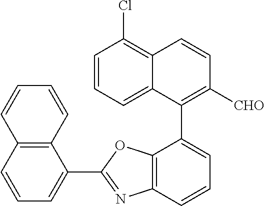 [SMILES: O=Cc1ccc2c(Cl)cccc2c1-c1cccc2nc(-c3cccc4ccccc34)oc12] | 71 |
| Sub- d9 | 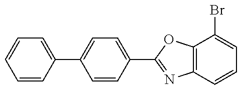 [SMILES: Brc1cccc2nc(-c3ccc(-c4ccccc4)cc3)oc12] | 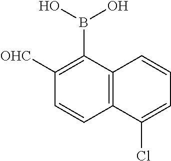 [SMILES: O=Cc1ccc2c(Cl)cccc2c1B(O)O] | 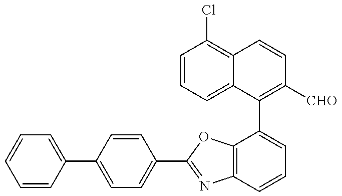 [SMILES: O=Cc1ccc2c(Cl)cccc2c1-c1cccc2nc(-c3ccc(-c4ccccc4)cc3)oc12] | 71 |
| Sub- d10 | 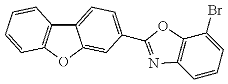 [SMILES: Brc1cccc2nc(-c3ccc4c(c3)oc3ccccc34)oc12] | 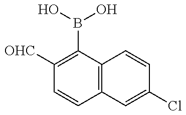 [SMILES: O=Cc1ccc2cc(Cl)ccc2c1B(O)O] | 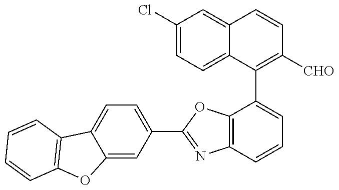 [SMILES: O=Cc1ccc2cc(Cl)ccc2c1-c1cccc2nc(-c3ccc4c(c3)oc3ccccc34)oc12] | 75 |
| Sub- d11 | 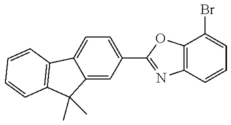 [SMILES: CC1(C)c2ccccc2-c2ccc(-c3nc4cccc(Br)c4o3)cc21] | 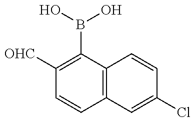 [SMILES: O=Cc1ccc2cc(Cl)ccc2c1B(O)O] | 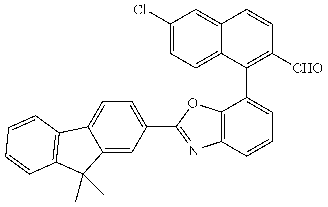 [SMILES: CC1(C)c2ccccc2-c2ccc(-c3nc4cccc(-c5c(C=O)ccc6cc(Cl)ccc56)c4o3)cc21] | 70 |
| Sub- d12 | 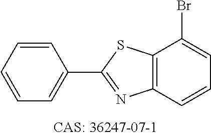 [SMILES: Brc1cccc2nc(-c3ccccc3)sc12] | 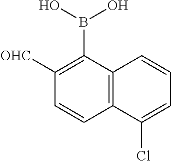 [SMILES: O=Cc1ccc2c(Cl)cccc2c1B(O)O] | 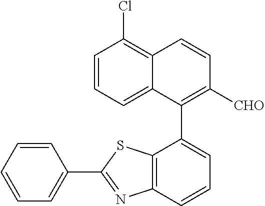 [SMILES: O=Cc1ccc2c(Cl)cccc2c1-c1cccc2nc(-c3ccccc3)sc12] | 73 |

### Synthesis of Sub-e1:

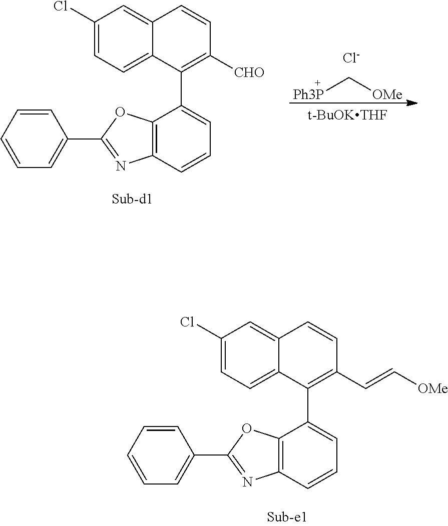

Under a nitrogen atmosphere, to a 1000 mL three-necked flask was added Sub-d1 (49.9 g, 130 mmol), (methoxymethyl) triphenylphosphonium chloride (74.38 g, 217 mmol) and anhydrous tetrahydrofuran (500 mL) in turn, and the resulting mixture was cooled to 0° C. by ice water bath. Then to the resulting mixture was slowly added potassium tert-butoxide in anhydrous tetrahydrofuran (1M, 220 mL). After the dropwise addition was completed, the resulting mixture was slowly heated to room temperature, and stirred for 6 hours. The reaction solution was poured into 1000 mL deionized water and extracted with ethyl acetate (250 mL×3 times). The organic phases were combined and dried with anhydrous magnesium sulfate followed by filtration and then reduced-pressure distillation to remove the solvent, obtaining a crude product. The crude product was purified by silica gel column chromatography with n-heptane as the mobile phase, to obtain a solid Sub-e1 (47.12 g, yield 88%).

Referring to the synthesis of Sub-e1, Sub-e2 to Sub-e12 were synthesized by replacing Sub-d1 with Reactant F as shown in Table 5.

TABLE 5
Synthesis of Sub-e2 to Sub-e12

| Sub-eX | Structure of | Structure of Sub- | Yield |
|---|---|---|---|
| number | Reactant F | eX | (%) |
| Sub-e2 | 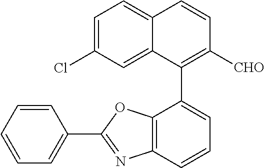 [SMILES: O=Cc1ccc2ccc(Cl)cc2c1-c1cccc2nc(-c3ccccc3)oc12] | 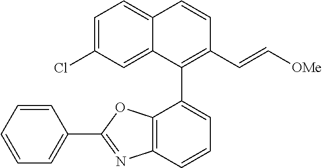 [SMILES: CO/C=C/c1ccc2ccc(Cl)cc2c1-c1cccc2nc(-c3ccccc3)oc12] | 81 |
| Sub-e3 | 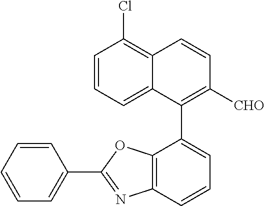 [SMILES: O=Cc1ccc2c(Cl)cccc2c1-c1cccc2nc(-c3ccccc3)oc12] | 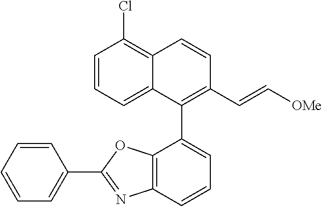 [SMILES: CO/C=C/c1ccc2c(Cl)cccc2c1-c1cccc2nc(-c3ccccc3)oc12] | 81 |
| Sub-e4 | 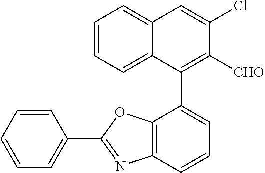 [SMILES: O=Cc1c(Cl)cc2ccccc2c1-c1cccc2nc(-c3ccccc3)oc12] | 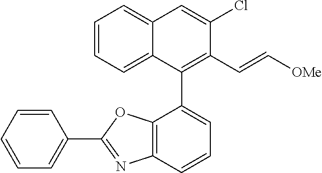 [SMILES: CO/C=C/c1c(Cl)cc2ccccc2c1-c1cccc2nc(-c3ccccc3)oc12] | 90 |
| Sub-e5 | 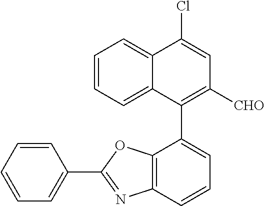 [SMILES: O=Cc1cc(Cl)c2ccccc2c1-c1cccc2nc(-c3ccccc3)oc12] | 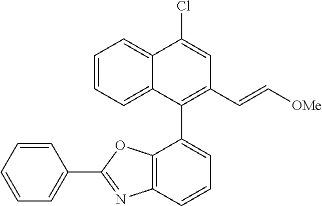 [SMILES: CO/C=C/c1cc(Cl)c2ccccc2c1-c1cccc2nc(-c3ccccc3)oc12] | 90 |
| Sub-e6 | 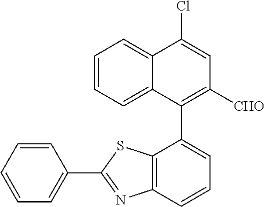 [SMILES: O=Cc1cc(Cl)c2ccccc2c1-c1cccc2nc(-c3ccccc3)sc12] | 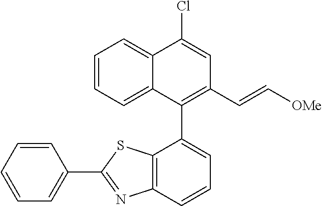 [SMILES: CO/C=C/c1cc(Cl)c2ccccc2c1-c1cccc2nc(-c3ccccc3)sc12] | 83 |
| Sub-e7 | 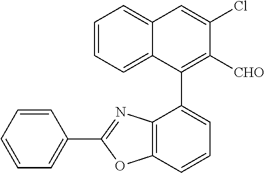 [SMILES: O=Cc1c(Cl)cc2ccccc2c1-c1cccc2oc(-c3ccccc3)nc12] | 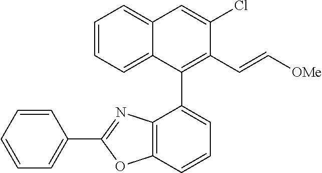 [SMILES: CO/C=C/c1c(Cl)cc2ccccc2c1-c1cccc2oc(-c3ccccc3)nc12] | 84 |
| Sub-e8 | 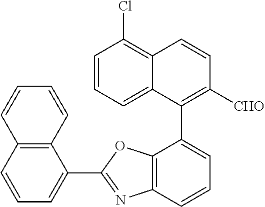 [SMILES: O=Cc1ccc2c(Cl)cccc2c1-c1cccc2nc(-c3cccc4ccccc34)oc12] | 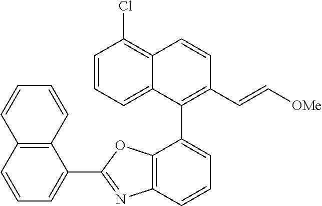 [SMILES: CO/C=C/c1ccc2c(Cl)cccc2c1-c1cccc2nc(-c3cccc4ccccc34)oc12] | 83 |
| Sub-e9 | 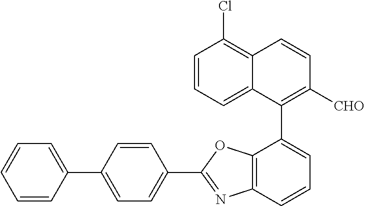 [SMILES: O=Cc1ccc2c(Cl)cccc2c1-c1cccc2nc(-c3ccc(-c4ccccc4)cc3)oc12] | 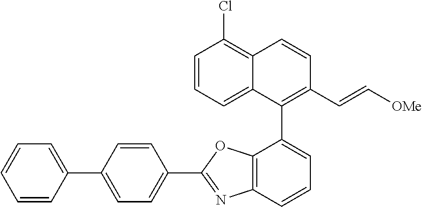 [SMILES: CO/C=C/c1ccc2c(Cl)cccc2c1-c1cccc2nc(-c3ccc(-c4ccccc4)cc3)oc12] | 90 |
| Sub-e10 | 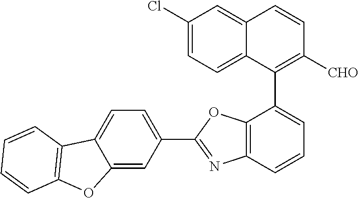 [SMILES: O=Cc1ccc2cc(Cl)ccc2c1-c1cccc2nc(-c3ccc4c(c3)oc3ccccc34)oc12] | 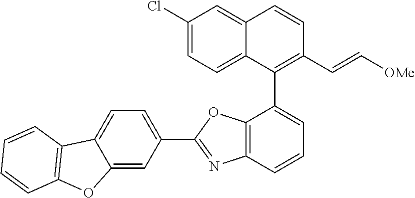 [SMILES: CO/C=C/c1ccc2cc(Cl)ccc2c1-c1cccc2nc(-c3ccc4c(c3)oc3ccccc34)oc12] | 87 |
| Sub-e11 | 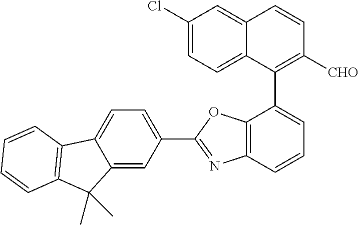 [SMILES: CC1(C)c2ccccc2-c2ccc(-c3nc4cccc(-c5c(C=O)ccc6cc(Cl)ccc56)c4o3)cc21] | 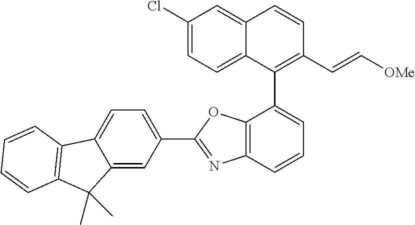 [SMILES: CO/C=C/c1ccc2cc(Cl)ccc2c1-c1cccc2nc(-c3ccc4c(c3)C(C)(C)c3ccccc3-4)oc12] | 84 |
| Sub-e12 | 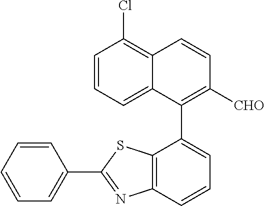 [SMILES: O=Cc1ccc2c(Cl)cccc2c1-c1cccc2nc(-c3ccccc3)sc12] | 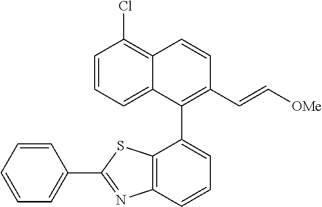 [SMILES: CO/C=C/c1ccc2c(Cl)cccc2c1-c1cccc2nc(-c3ccccc3)sc12] | 90 |

### Synthesis of Sub-f1:

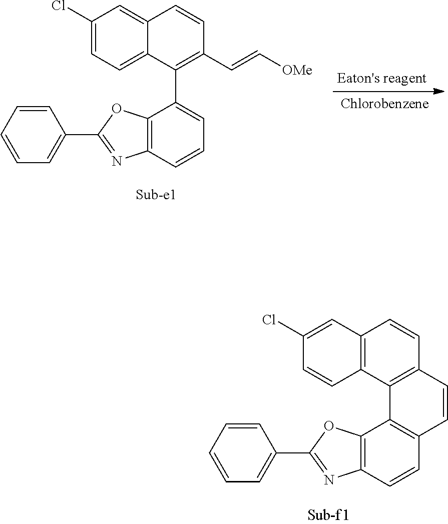 [SMILES: CO/C=C/c1ccc2cc(Cl)ccc2c1-c1cccc2nc(-c3ccccc3)oc12 ; Clc1ccc2c(ccc3ccc4ccc5nc(-c6ccccc6)oc5c4c32)c1]

Under a nitrogen atmosphere, to a 1000 mL three-necked flask was added Sub-e1 (49.0 g, 119 mmol), Eaton's reagent (4.5 mL) and chlorobenzene (500 mL) in turn, and the resulting mixture was heated to reflux and stirred for 4 h. After the reaction solution was cooled to room temperature, it was poured into 1000 ml deionized water, and neutralized with saturated sodium hydroxide solution, and then extracted with dichloromethane (250 mL×3 times). The organic phases were combined and dried with anhydrous magnesium sulfate followed by filtration and then reduced-pressure distillation to remove the solvent, obtaining a crude product. The crude product was purified by silica gel column chromatography with a mixture of dichloromethane and n-heptane as mobile phase, obtaining a solid Sub-f1 (33.0 g, yield 73%).

Referring to the synthesis of Sub-f1, Sub-f2 to Sub-f12 were synthesized by replacing Sub-e1 with Reactant G as shown in Table 6.

TABLE 6
Synthesis of Sub-f2 to Sub-f12

| Sub-fX | | Structure of | Yield |
|---|---|---|---|
| number | Reactant G | Sub-fX | (%) |
| Sub-f2 | 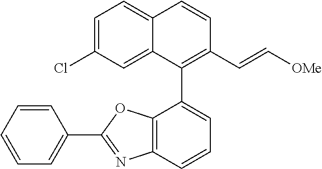 [SMILES: CO/C=C/c1ccc2ccc(Cl)cc2c1-c1cccc2nc(-c3ccccc3)oc12] | 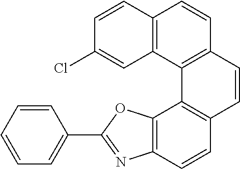 [SMILES: Clc1ccc2ccc3ccc4ccc5nc(-c6ccccc6)oc5c4c3c2c1] | 65 |
| Sub-f3 | 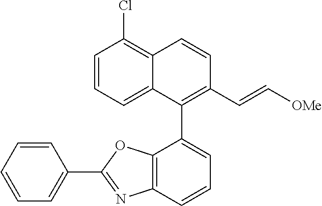 [SMILES: CO/C=C/c1ccc2c(Cl)cccc2c1-c1cccc2nc(-c3ccccc3)oc12] | 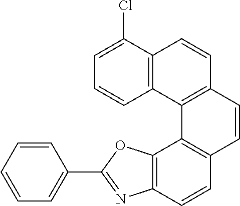 [SMILES: Clc1cccc2c1ccc1ccc3ccc4nc(-c5ccccc5)oc4c3c12] | 68 |
| Sub-f4 | 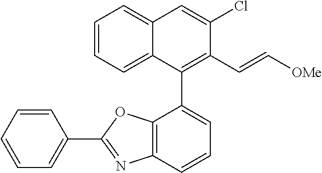 [SMILES: CO/C=C/c1c(Cl)cc2ccccc2c1-c1cccc2nc(-c3ccccc3)oc12] | 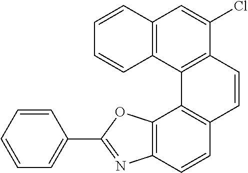 [SMILES: Clc1cc2ccccc2c2c1ccc1ccc3nc(-c4ccccc4)oc3c12] | 74 |
| Sub-f5 | 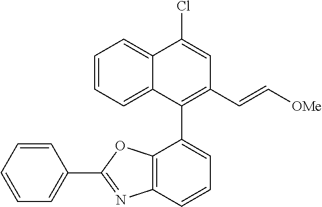 [SMILES: CO/C=C/c1cc(Cl)c2ccccc2c1-c1cccc2nc(-c3ccccc3)oc12] | 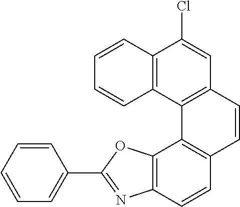 [SMILES: Clc1cc2ccc3ccc4nc(-c5ccccc5)oc4c3c2c2ccccc12] | 72 |
| Sub-f6 | 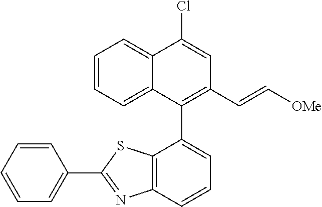 [SMILES: CO/C=C/c1cc(Cl)c2ccccc2c1-c1cccc2nc(-c3ccccc3)sc12] | 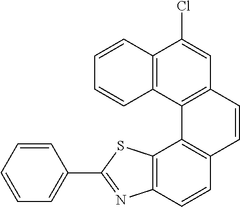 [SMILES: Clc1cc2ccc3ccc4nc(-c5ccccc5)sc4c3c2c2ccccc12] | 72 |
| Sub-f7 | 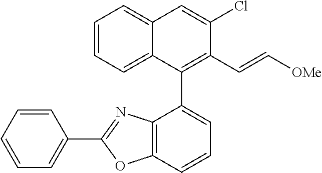 [SMILES: CO/C=C/c1c(Cl)cc2ccccc2c1-c1cccc2oc(-c3ccccc3)nc12] | 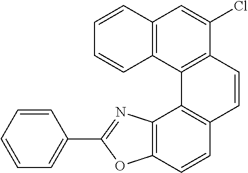 [SMILES: Clc1cc2ccccc2c2c1ccc1ccc3oc(-c4ccccc4)nc3c12] | 73 |
| Sub-f8 | 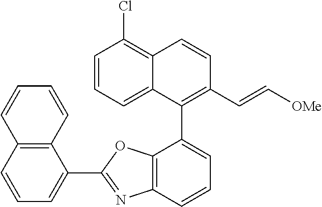 [SMILES: CO/C=C/c1ccc2c(Cl)cccc2c1-c1cccc2nc(-c3cccc4ccccc34)oc12] | 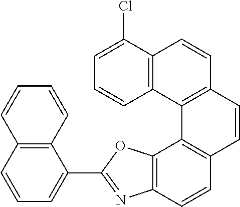 [SMILES: Clc1cccc2c1ccc1ccc3ccc4nc(-c5cccc6ccccc56)oc4c3c12] | 67 |
| Sub-f9 | 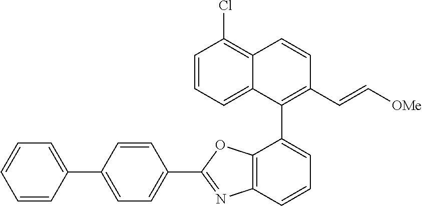 [SMILES: CO/C=C/c1ccc2c(Cl)cccc2c1-c1cccc2nc(-c3ccc(-c4ccccc4)cc3)oc12] | 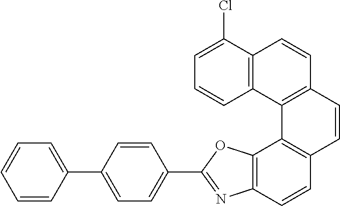 [SMILES: Clc1cccc2c1ccc1ccc3ccc4nc(-c5ccc(-c6ccccc6)cc5)oc4c3c12] | 71 |
| Sub-f10 | 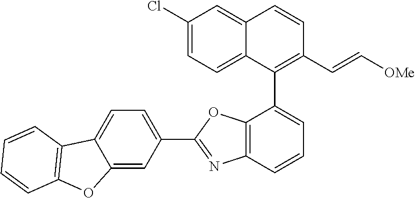 [SMILES: CO/C=C/c1ccc2cc(Cl)ccc2c1-c1cccc2nc(-c3ccc4c(c3)oc3ccccc34)oc12] | 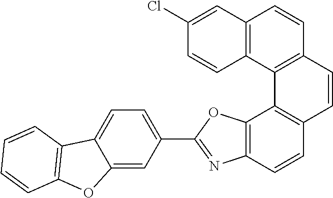 [SMILES: Clc1ccc2c(ccc3ccc4ccc5nc(-c6ccc7c(c6)oc6ccccc67)oc5c4c32)c1] | 67 |
| Sub-f11 | 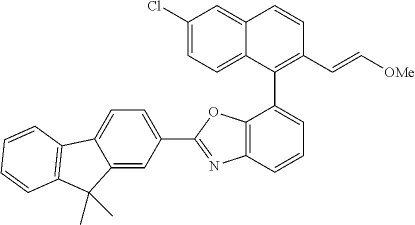 [SMILES: CO/C=C/c1ccc2cc(Cl)ccc2c1-c1cccc2nc(-c3ccc4c(c3)C(C)(C)c3ccccc3-4)oc12] | 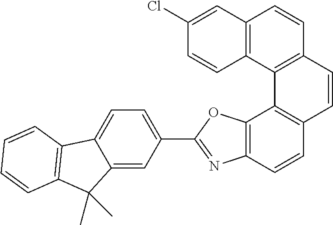 [SMILES: CC1(C)c2ccccc2-c2ccc(-c3nc4ccc5ccc6ccc7cc(Cl)ccc7c6c5c4o3)cc21] | 70 |
| Sub-f12 | 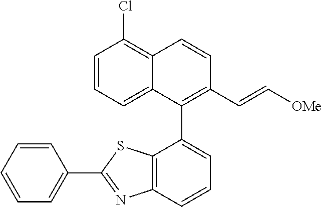 [SMILES: CO/C=C/c1ccc2c(Cl)cccc2c1-c1cccc2nc(-c3ccccc3)sc12] | 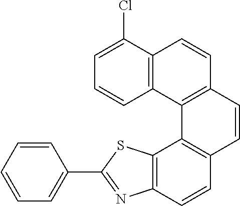 [SMILES: Clc1cccc2c1ccc1ccc3ccc4nc(-c5ccccc5)sc4c3c12] | 69 |

### Synthesis of Sub-f13:

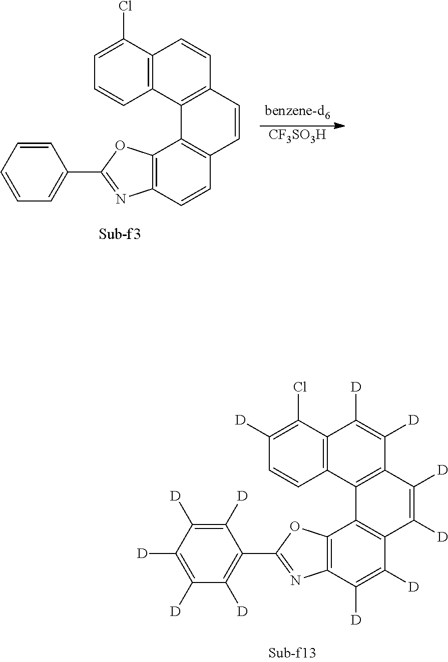

Under a nitrogen atmosphere, to a 100 mL three-necked flask was added Sub-f3 (9.49 g, 25 mmol) and 200 mL benzene-D₆ and the resulting mixture was heated to 60° C. Trifluoromethanesulfonic acid (22.51 g, 150 mmol) was added to the resulting mixture, and the heating was continued until boiling, and the reaction was proceeded for 24 hours at stirring. After the reaction solution was cooled to room temperature, 50 mL of deuterium oxide was added and stirred for 10 minutes, and saturated K₃PO₄ aqueous solution was added to neutralize the reaction solution. The organic layer was extracted with dichloromethane (50 mL×3 times), and the organic phases were combined and dried with anhydrous sodium sulfate followed by filtration and then reduced-pressure distillation to remove the solvent, obtaining a crude product. The crude product was purified by silica gel column chromatography using n-heptane/dichloromethane as mobile phase, obtaining a white solid Sub-f13 (5.39 g, yield 55%).

### Synthesis of Sub-g1:

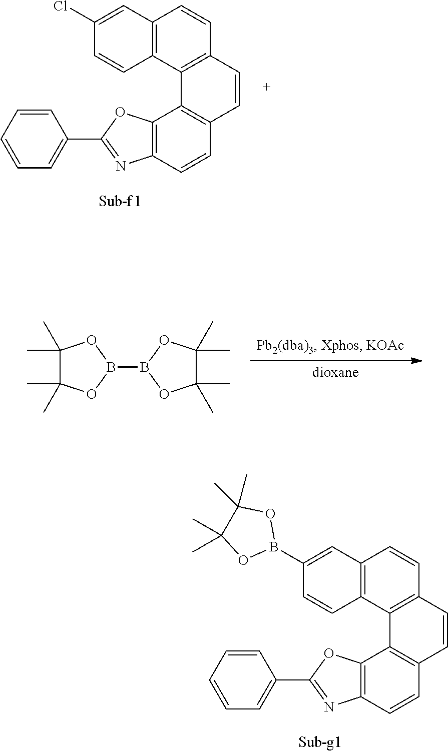

Under a nitrogen atmosphere, to a 500 mL three-necked flask was added Sub-f1 (16.7 g, 44 mmol), biboronic acid pinacol ester (12.28 g, 48.4 mmol), potassium acetate (9.50 g, 96.8 mmol) and 1,4-dioxane (120 mL), stirring and heating were initiated, and tris (dibenzylideneacetone) dipalladium (0.40 g, 0.44 mmol) and (2-dicyclohexylphosphine-2′, 4′, 6′-triisopropylbiphenyl) (0.42 g, 0.88 mmol) were quickly added when the resulting mixture was heated to 40° C. The temperature was continued to rise to reflux, and the reaction solution was stirred overnight. After the reaction solution was cooled to room temperature, 200 mL water was added and fully stirred for 30 minutes. The resulting solution was filtered under reduced pressure, and the filter cake was washed with deionized water until neutral, and then washed with 100 mL anhydrous ethanol, to get a gray solid. The crude product was beaten with n-heptane once, and then dissolved in 200 mL toluene, and passed through a silica gel column to remove the catalyst, obtaining a white solid Sub-g1 (15.14 g, yield 73%) after concentration.

Referring to the synthesis of Sub-g1, Sub-g2 to Sub-g13 were synthesized by replacing Sub-f1 with Reactant H as shown in Table 7.

TABLE 7
Synthesis of Sub-g2 to Sub-g13

| Sub-gX | | Structure of Sub- | Yield |
|---|---|---|---|
| number | Reactant H | gX | (%) |
| Sub-g2 | 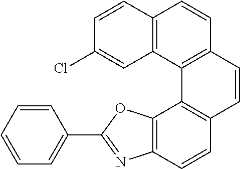 [SMILES: Clc1ccc2ccc3ccc4ccc5nc(-c6ccccc6)oc5c4c3c2c1] | 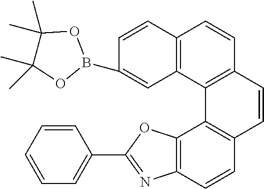 [SMILES: CC1(C)OB(c2ccc3ccc4ccc5ccc6nc(-c7ccccc7)oc6c5c4c3c2)OC1(C)C] | 61 |
| Sub-g3 | 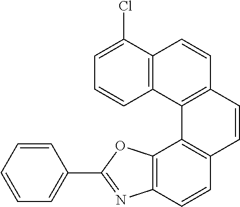 [SMILES: Clc1cccc2c1ccc1ccc3ccc4nc(-c5ccccc5)oc4c3c12] | 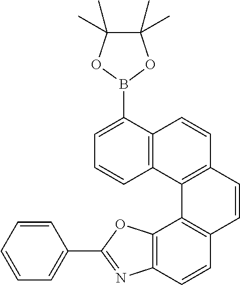 [SMILES: CC1(C)OB(c2cccc3c2ccc2ccc4ccc5nc(-c6ccccc6)oc5c4c23)OC1(C)C] | 71 |
| Sub-g4 | 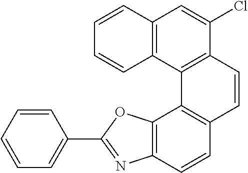 [SMILES: Clc1cc2ccccc2c2c1ccc1ccc3nc(-c4ccccc4)oc3c12] | 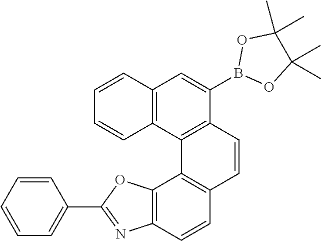 [SMILES: CC1(C)OB(c2cc3ccccc3c3c2ccc2ccc4nc(-c5ccccc5)oc4c23)OC1(C)C] | 78 |
| Sub-g5 | 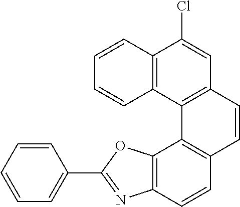 [SMILES: Clc1cc2ccc3ccc4nc(-c5ccccc5)oc4c3c2c2ccccc12] | 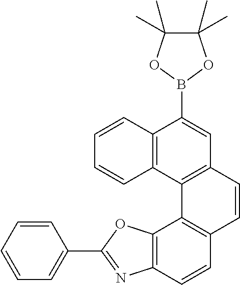 [SMILES: CC1(C)OB(c2cc3ccc4ccc5nc(-c6ccccc6)oc5c4c3c3ccccc23)OC1(C)C] | 75 |
| Sub-g6 | 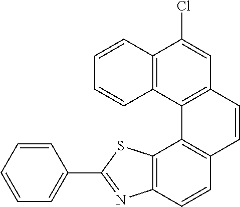 [SMILES: Clc1cc2ccc3ccc4nc(-c5ccccc5)sc4c3c2c2ccccc12] | 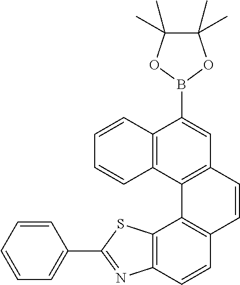 [SMILES: CC1(C)OB(c2cc3ccc4ccc5nc(-c6ccccc6)sc5c4c3c3ccccc23)OC1(C)C] | 73 |
| Sub-g7 | 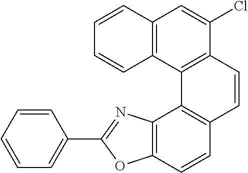 [SMILES: Clc1cc2ccccc2c2c1ccc1ccc3oc(-c4ccccc4)nc3c12] | 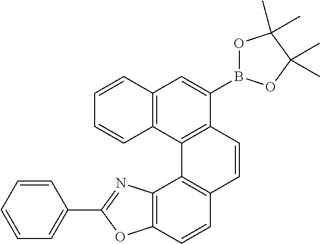 [SMILES: CC1(C)OB(c2cc3ccccc3c3c2ccc2ccc4oc(-c5ccccc5)nc4c23)OC1(C)C] | 70 |
| Sub-g8 | 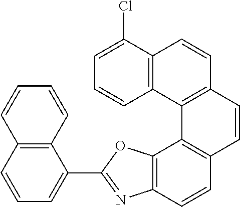 [SMILES: Clc1cccc2c1ccc1ccc3ccc4nc(-c5cccc6ccccc56)oc4c3c12] | 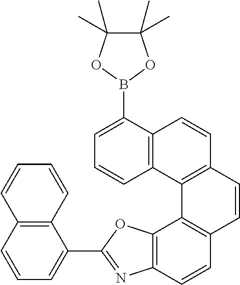 [SMILES: CC1(C)OB(c2cccc3c2ccc2ccc4ccc5nc(-c6cccc7ccccc67)oc5c4c23)OC1(C)C] | 80 |
| Sub-g9 | 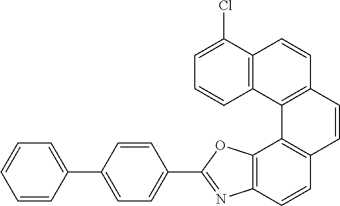 [SMILES: Clc1cccc2c1ccc1ccc3ccc4nc(-c5ccc(-c6ccccc6)cc5)oc4c3c12] | 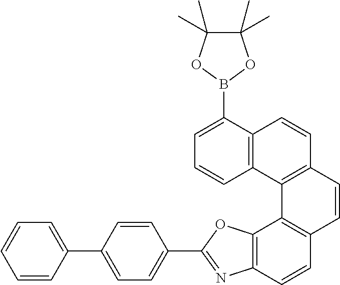 [SMILES: CC1(C)OB(c2cccc3c2ccc2ccc4ccc5nc(-c6ccc(-c7ccccc7)cc6)oc5c4c23)OC1(C)C] | 68 |
| Sub-g10 | 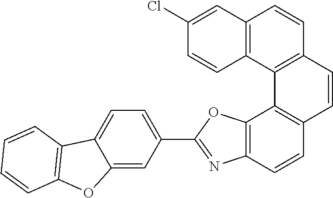 [SMILES: Clc1ccc2c(ccc3ccc4ccc5nc(-c6ccc7c(c6)oc6ccccc67)oc5c4c32)c1] | 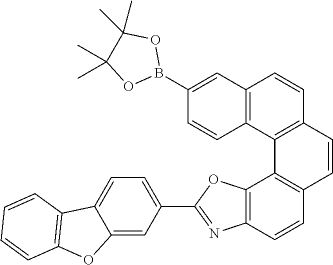 [SMILES: CC1(C)OB(c2ccc3c(ccc4ccc5ccc6nc(-c7ccc8c(c7)oc7ccccc78)oc6c5c43)c2)OC1(C)C] | 68 |
| Sub-g11 | 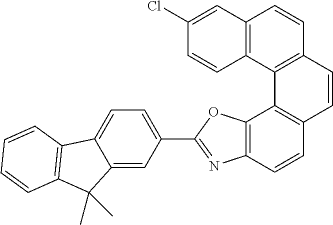 [SMILES: CC1(C)c2ccccc2-c2ccc(-c3nc4ccc5ccc6ccc7cc(Cl)ccc7c6c5c4o3)cc21] | 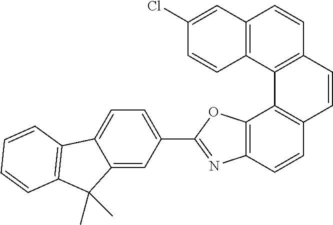 [SMILES: CC1(C)c2ccccc2-c2ccc(-c3nc4ccc5ccc6ccc7cc(Cl)ccc7c6c5c4o3)cc21] | 73 |
| Sub-g12 | 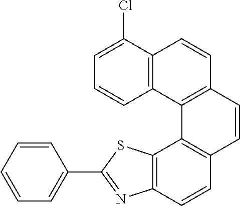 [SMILES: Clc1cccc2c1ccc1ccc3ccc4nc(-c5ccccc5)sc4c3c12] | 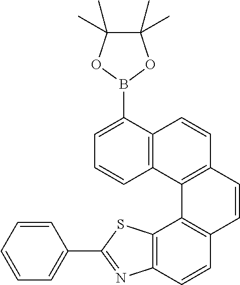 [SMILES: CC1(C)OB(c2cccc3c2ccc2ccc4ccc5nc(-c6ccccc6)sc5c4c23)OC1(C)C] | 69 |
| Sub-g13 | 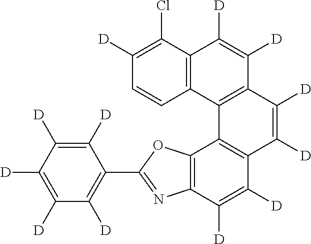 [SMILES: [2H]c1ccc2c(c([2H])c([2H])c3c([2H])c([2H])c4c([2H])c([2H])c5nc(-c6c([2H])c([2H])c([2H])c([2H])c6[2H])oc5c4c32)c1Cl] | 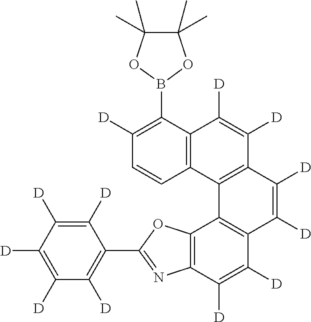 [SMILES: [2H]c1ccc2c(c([2H])c([2H])c3c([2H])c([2H])c4c([2H])c([2H])c5nc(-c6c([2H])c([2H])c([2H])c([2H])c6[2H])oc5c4c32)c1B1OC(C)(C)C(C)(C)O1] | 66 |

### Synthesis of Sub-h1:

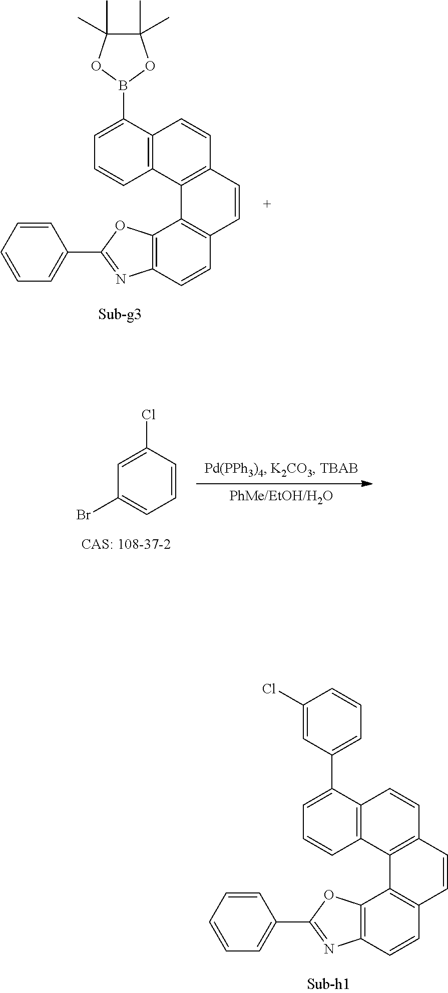 [SMILES: CC1(C)OB(c2cccc3c2ccc2ccc4ccc5nc(-c6ccccc6)oc5c4c23)OC1(C)C ; Clc1cccc(Br)c1 ; Clc1cccc(-c2cccc3c2ccc2ccc4ccc5nc(-c6ccccc6)oc5c4c23)c1]

Under a nitrogen atmosphere, m-chlorobromobenzene (4.78 g, 25 mmol), Sub-g3 (12.96 g, 27.5 mmol), tetrakis (triphenylphosphine) palladium (0.29 g, 0.25 mmol), anhydrous potassium carbonate (6.9 g, 100 mmol), toluene (140 mL), anhydrous ethanol (35 mL) and deionized water (35 mL) were added to a 500 mL three-necked flask in turn, then the stirring and heating were initiated, and the resulting mixture was heated to reflux for 16 hours. After the reaction solution was cooled to room temperature, it was extracted with dichloromethane (100 mL×3 times), and the organic phases were combined and dried with anhydrous magnesium sulfate followed by filtration and reduced-pressure distillation to remove the solvent, obtaining a crude product. The crude product was purified by silica gel column chromatography with n-heptane as the mobile phase, obtaining white solid Sub-h1 (9.8 g, yield 86%).

Referring to the synthesis of Sub-h1, Sub-h2 to Sub-h4 were synthesized by replacing m-chlorobromobenzene with Reactant J, and replacing Sub-g3 with Reactant K as shown in Table 8.

TABLE 8
Synthesis of Sub-h2 to Sub-h4

| Sub- | | | | |
|---|---|---|---|---|
| hX | | | | |
| num- | | | Structure of Sub- | Yield |
| ber | Reactant J | Reactant K | hX | (%) |
| Sub- h2 | 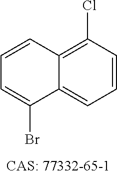 [SMILES: Clc1cccc2c(Br)cccc12] | 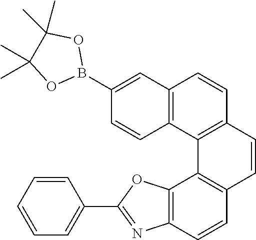 [SMILES: CC1(C)OB(c2ccc3c(ccc4ccc5ccc6nc(-c7ccccc7)oc6c5c43)c2)OC1(C)C] | 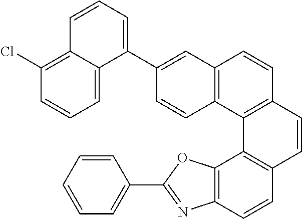 [SMILES: Clc1cccc2c(-c3ccc4c(ccc5ccc6ccc7nc(-c8ccccc8)oc7c6c54)c3)cccc12] | 82 |
| Sub- h3 | 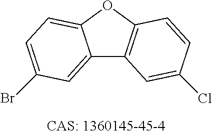 [SMILES: Clc1ccc2oc3ccc(Br)cc3c2c1] | 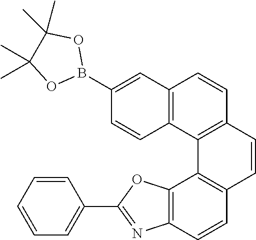 [SMILES: CC1(C)OB(c2ccc3c(ccc4ccc5ccc6nc(-c7ccccc7)oc6c5c43)c2)OC1(C)C] | 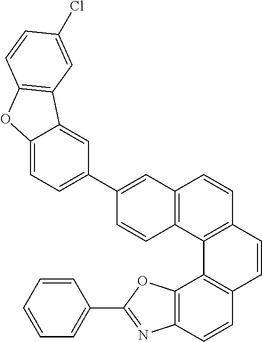 [SMILES: Clc1ccc2oc3ccc(-c4ccc5c(ccc6ccc7ccc8nc(-c9ccccc9)oc8c7c65)c4)cc3c2c1] | 81 |
| Sub- h4 | 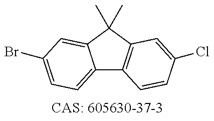 [SMILES: CC1(C)c2cc(Cl)ccc2-c2ccc(Br)cc21] | 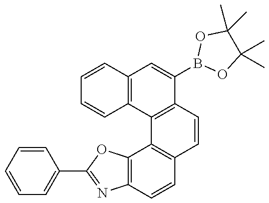 [SMILES: CC1(C)OB(c2cc3ccccc3c3c2ccc2ccc4nc(-c5ccccc5)oc4c23)OC1(C)C] | 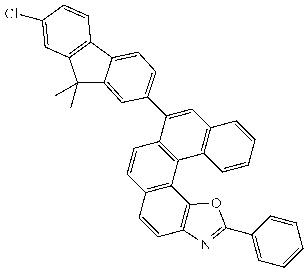 [SMILES: CC1(C)c2cc(Cl)ccc2-c2ccc(-c3cc4ccccc4c4c3ccc3ccc5nc(-c6ccccc6)oc5c34)cc21] | 78 |

### Synthesis of Compound A3:

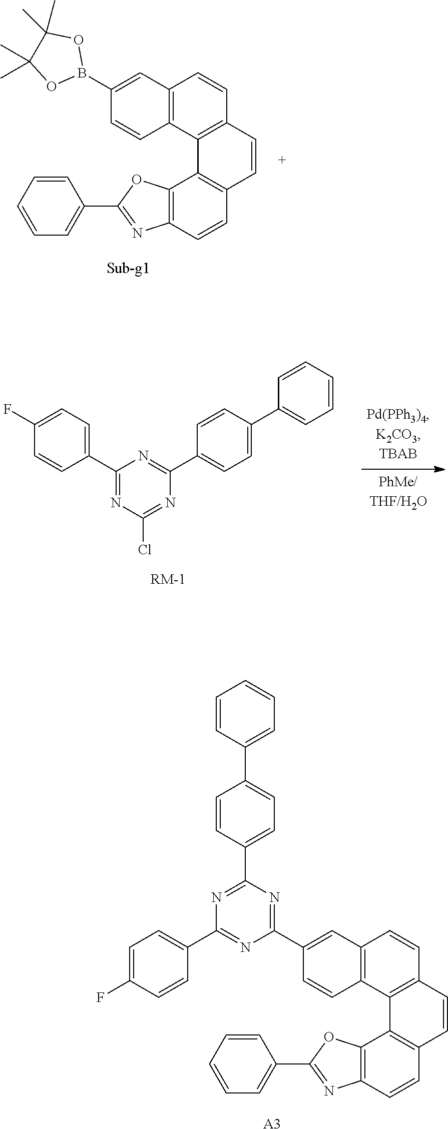

Under a nitrogen atmosphere, to a 250 mL three-necked flask was added Sub-g1 (11.78 g, 25 mmol), RM-1 (CAS: 2737218-48-1, 9.0 g, 25 mmol), tetrakis (triphenylphosphine) palladium (0.29 g, 0.25 mmol), anhydrous potassium carbonate (6.9 g, 50 mmol), tetrabutylammonium bromide (0.8 g, 2.5 mmol), toluene (120 mL), tetrahydrofuran (30 mL) and deionized water (30 mL) in turn, and stirring and heating were initiated. The resulting mixture was heated to reflux for 16 h. After the reaction solution was cooled to room temperature, it was extracted with dichloromethane (100 mL×3 times), and the organic phases were combined and dried with anhydrous magnesium sulfate followed by filtration and then reduced pressure distillation to remove the solvent, to get a crude product. The crude product was purified by silica gel column chromatography with a mixture of dichloromethane and n-heptane as mobile phase, obtaining a white solid Compound A3 (12.24 g, yield 73%, m/z=671.2 [M+H]⁺).

Referring to the synthesis of Compound A3, the compounds of the present disclosure in Table 9 were synthesized by replacing Sub-g1 with Reactant L, and replacing RM-1 with Reactant M as shown in Table 9.

TABLE 9
Synthesis of the compounds of the present disclosure

| | | | m/z | |
|---|---|---|---|---|
| | | Compound structure | ([M + | Yield |
| Reactant L | Reactant M | and number | H]⁺) | (%) |
| 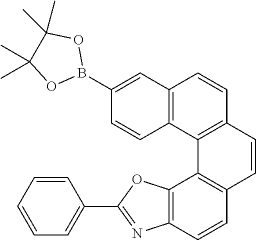 [SMILES: CC1(C)OB(c2ccc3c(ccc4ccc5ccc6nc(-c7ccccc7)oc6c5c43)c2)OC1(C)C] | 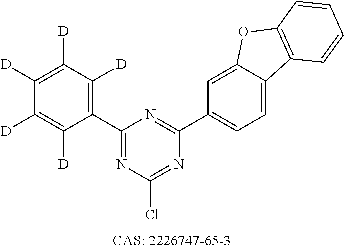 [SMILES: [2H]c1c([2H])c([2H])c(-c2nc(Cl)nc(-c3ccc4c(c3)oc3ccccc34)n2)c([2H])c1[2H]] | 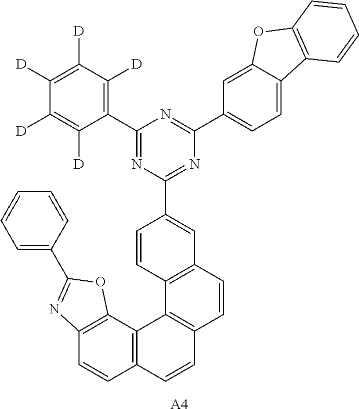 | 672.2 | 73 |
| 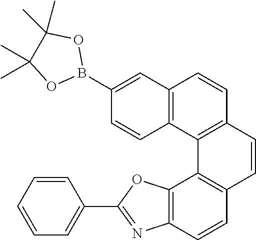 [SMILES: CC1(C)OB(c2ccc3c(ccc4ccc5ccc6nc(-c7ccccc7)oc6c5c43)c2)OC1(C)C] | 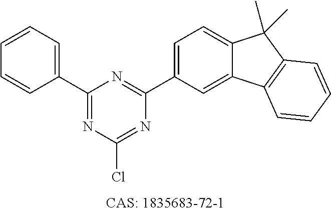 [SMILES: CC1(C)c2ccccc2-c2cc(-c3nc(Cl)nc(-c4ccccc4)n3)ccc21] | 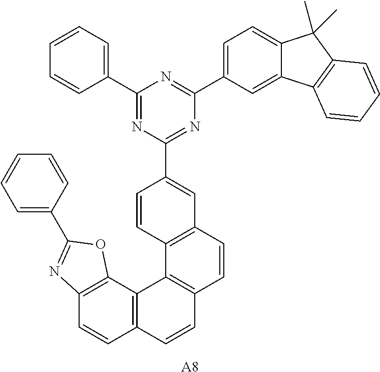 | 693.3 | 74 |
| 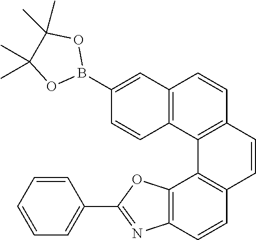 [SMILES: CC1(C)OB(c2ccc3c(ccc4ccc5ccc6nc(-c7ccccc7)oc6c5c43)c2)OC1(C)C] | 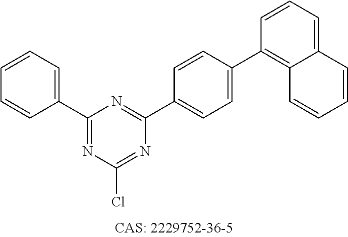 [SMILES: Clc1nc(-c2ccccc2)nc(-c2ccc(-c3cccc4ccccc34)cc2)n1] | 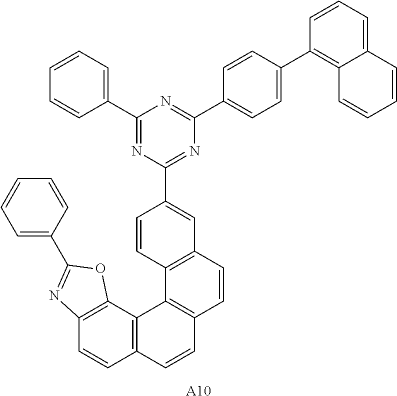 | 703.2 | 75 |
| 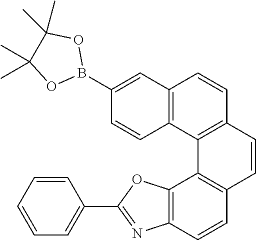 [SMILES: CC1(C)OB(c2ccc3c(ccc4ccc5ccc6nc(-c7ccccc7)oc6c5c43)c2)OC1(C)C] | 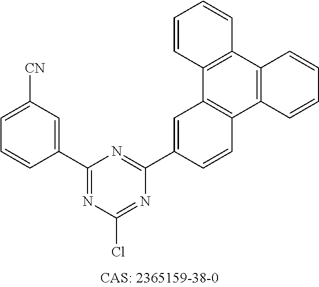 [SMILES: N#Cc1cccc(-c2nc(Cl)nc(-c3ccc4c5ccccc5c5ccccc5c4c3)n2)c1] | 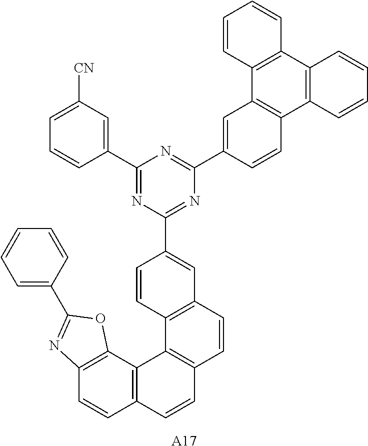 | 752.2 | 75 |
| 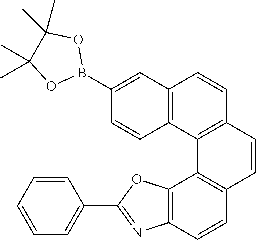 [SMILES: CC1(C)OB(c2ccc3c(ccc4ccc5ccc6nc(-c7ccccc7)oc6c5c43)c2)OC1(C)C] | 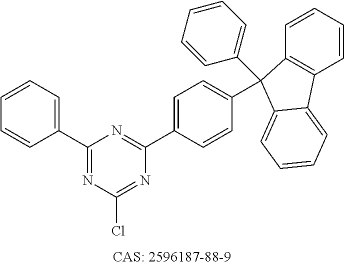 [SMILES: Clc1nc(-c2ccccc2)nc(-c2ccc(C3(c4ccccc4)c4ccccc4-c4ccccc43)cc2)n1] | 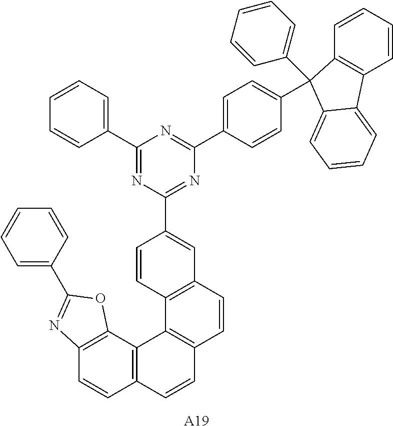 | 817.3 | 70 |
| 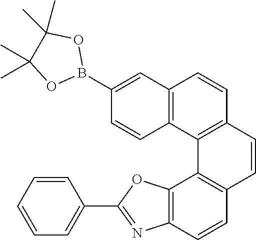 [SMILES: CC1(C)OB(c2ccc3c(ccc4ccc5ccc6nc(-c7ccccc7)oc6c5c43)c2)OC1(C)C] | 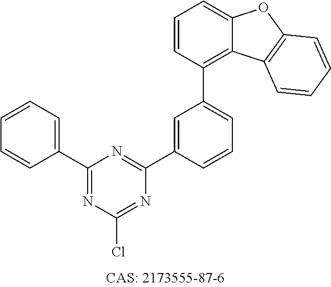 [SMILES: Clc1nc(-c2ccccc2)nc(-c2cccc(-c3cccc4oc5ccccc5c34)c2)n1] | 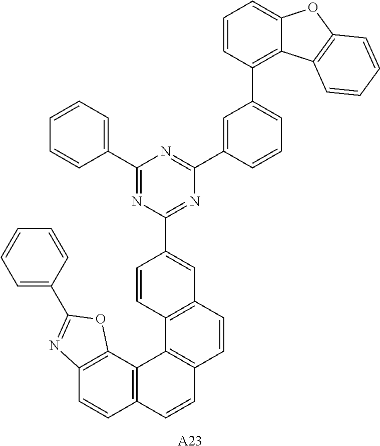 | 743.2 | 75 |
| 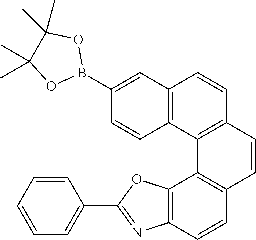 [SMILES: CC1(C)OB(c2ccc3c(ccc4ccc5ccc6nc(-c7ccccc7)oc6c5c43)c2)OC1(C)C] | 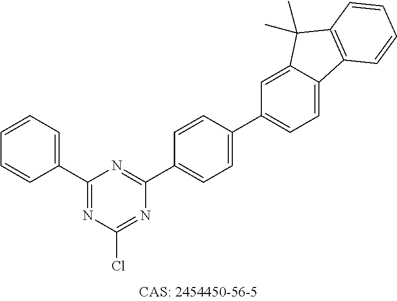 [SMILES: CC1(C)c2ccccc2-c2ccc(-c3ccc(-c4nc(Cl)nc(-c5ccccc5)n4)cc3)cc21] | 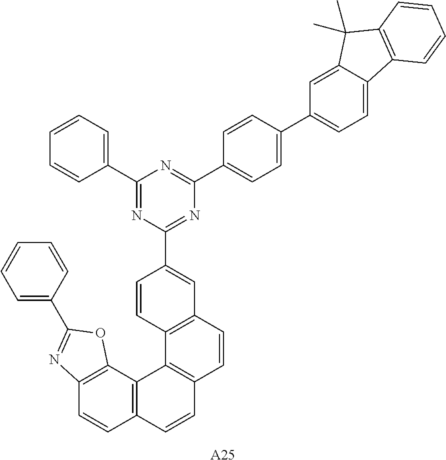 [SMILES: CC1(C)c2ccccc2-c2ccc(-c3ccc(-c4nc(-c5ccccc5)nc(-c5ccc6c(ccc7ccc8ccc9nc(-c%10ccccc%10)oc9c8c76)c5)n4)cc3)cc21 ; "A25"] | 769.3 | 70 |
| 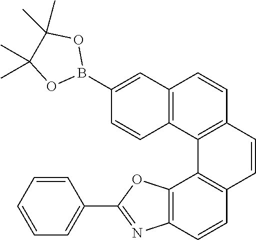 [SMILES: CC1(C)OB(c2ccc3c(ccc4ccc5ccc6nc(-c7ccccc7)oc6c5c43)c2)OC1(C)C] | 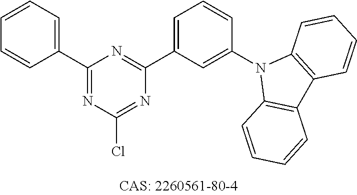 [SMILES: Clc1nc(-c2ccccc2)nc(-c2cccc(-n3c4ccccc4c4ccccc43)c2)n1] | 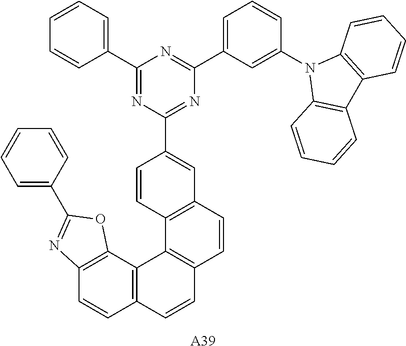 | 742.3 | 66 |
| 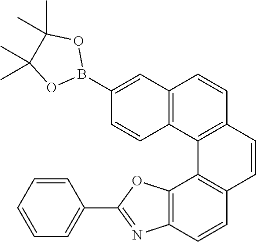 [SMILES: CC1(C)OB(c2ccc3c(ccc4ccc5ccc6nc(-c7ccccc7)oc6c5c43)c2)OC1(C)C] | 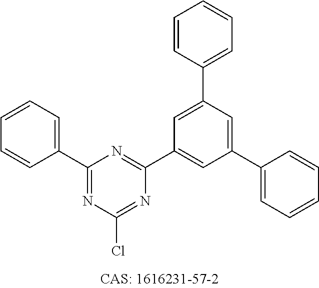 [SMILES: Clc1nc(-c2ccccc2)nc(-c2cc(-c3ccccc3)cc(-c3ccccc3)c2)n1] | 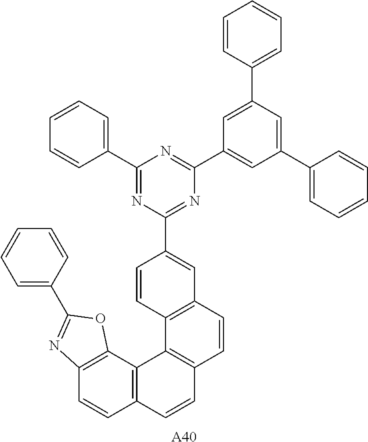 | 729.3 | 72 |
| 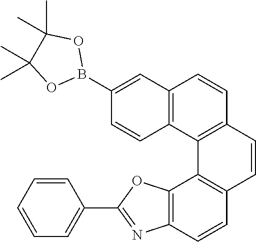 [SMILES: CC1(C)OB(c2ccc3c(ccc4ccc5ccc6nc(-c7ccccc7)oc6c5c43)c2)OC1(C)C] | 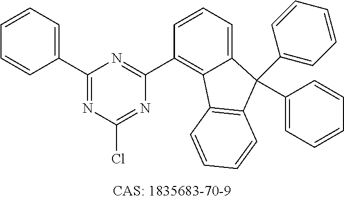 [SMILES: Clc1nc(-c2ccccc2)nc(-c2cccc3c2-c2ccccc2C3(c2ccccc2)c2ccccc2)n1] | 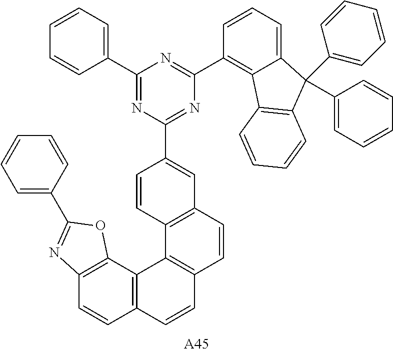 | 817.3 | 69 |
| 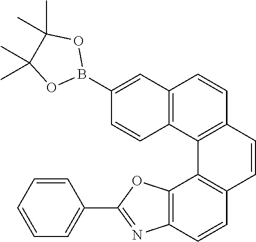 [SMILES: CC1(C)OB(c2ccc3c(ccc4ccc5ccc6nc(-c7ccccc7)oc6c5c43)c2)OC1(C)C] | 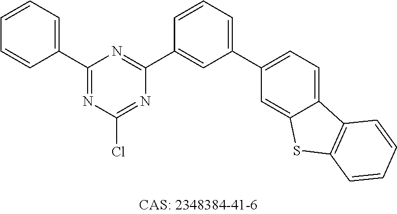 [SMILES: Clc1nc(-c2ccccc2)nc(-c2cccc(-c3ccc4c(c3)sc3ccccc34)c2)n1] | 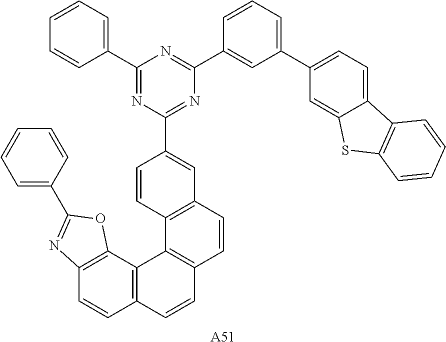 | 759.2 | 67 |
| 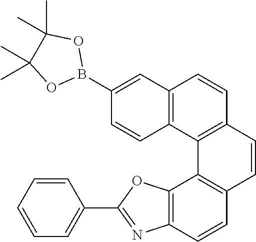 [SMILES: CC1(C)OB(c2ccc3c(ccc4ccc5ccc6nc(-c7ccccc7)oc6c5c43)c2)OC1(C)C] | 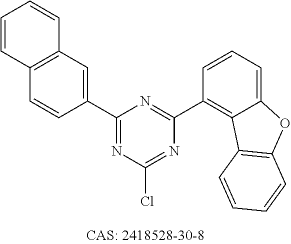 [SMILES: Clc1nc(-c2ccc3ccccc3c2)nc(-c2cccc3oc4ccccc4c23)n1] | 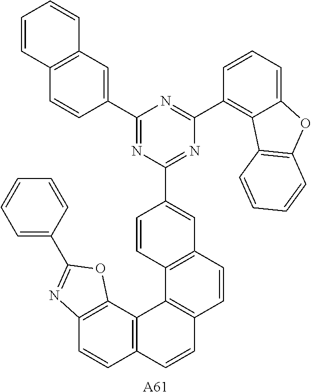 | 717.2 | 75 |
| 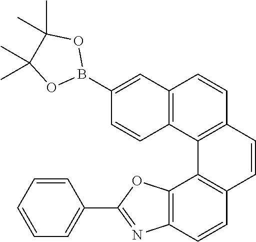 [SMILES: CC1(C)OB(c2ccc3c(ccc4ccc5ccc6nc(-c7ccccc7)oc6c5c43)c2)OC1(C)C] | 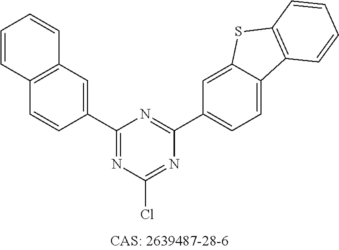 [SMILES: Clc1nc(-c2ccc3ccccc3c2)nc(-c2ccc3c(c2)sc2ccccc23)n1] | 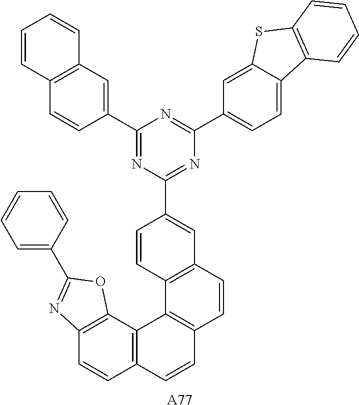 | 733.2 | 71 |
| 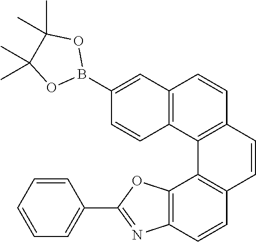 [SMILES: CC1(C)OB(c2ccc3c(ccc4ccc5ccc6nc(-c7ccccc7)oc6c5c43)c2)OC1(C)C] | 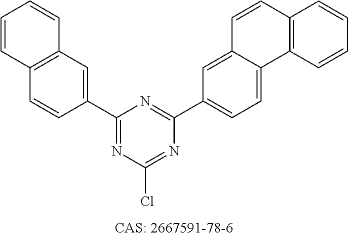 [SMILES: Clc1nc(-c2ccc3ccccc3c2)nc(-c2ccc3c(ccc4ccccc43)c2)n1] | 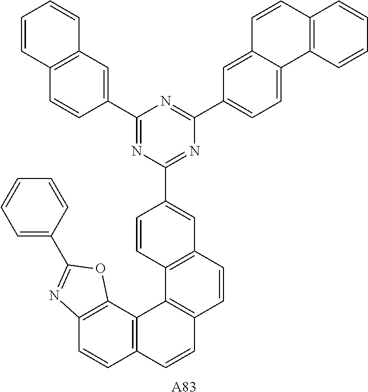 | 727.2 | 66 |
| 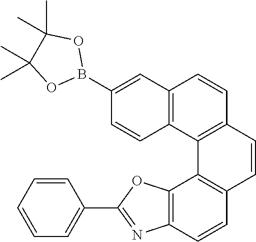 [SMILES: CC1(C)OB(c2ccc3c(ccc4ccc5ccc6nc(-c7ccccc7)oc6c5c43)c2)OC1(C)C] | 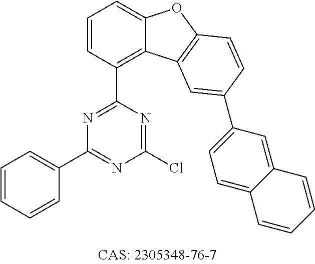 [SMILES: Clc1nc(-c2ccccc2)nc(-c2cccc3oc4ccc(-c5ccc6ccccc6c5)cc4c23)n1] | 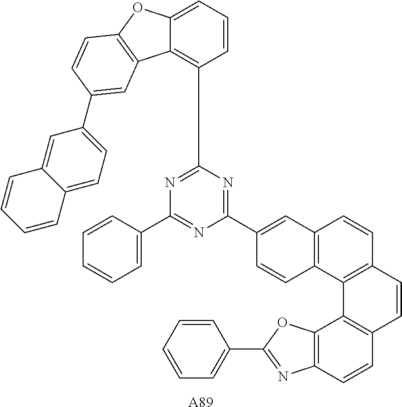 | 793.3 | 65 |
| 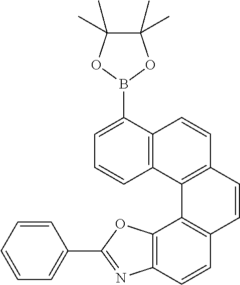 [SMILES: CC1(C)OB(c2cccc3c2ccc2ccc4ccc5nc(-c6ccccc6)oc5c4c23)OC1(C)C] | 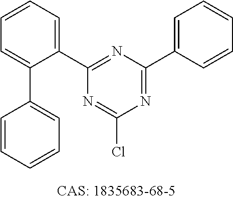 [SMILES: Clc1nc(-c2ccccc2)nc(-c2ccccc2-c2ccccc2)n1] | 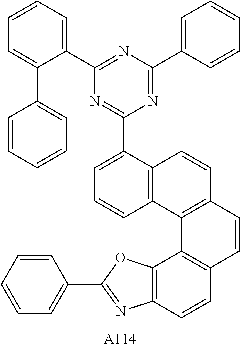 | 653.2 | 74 |
| 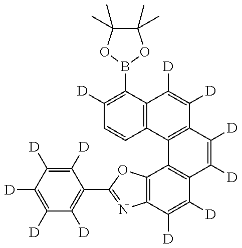 [SMILES: [2H]c1ccc2c(c([2H])c([2H])c3c([2H])c([2H])c4c([2H])c([2H])c5nc(-c6c([2H])c([2H])c([2H])c([2H])c6[2H])oc5c4c32)c1B1OC(C)(C)C(C)(C)O1] | 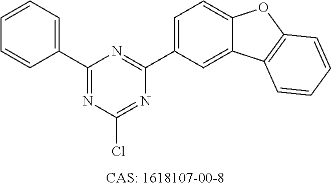 [SMILES: Clc1nc(-c2ccccc2)nc(-c2ccc3oc4ccccc4c3c2)n1] | 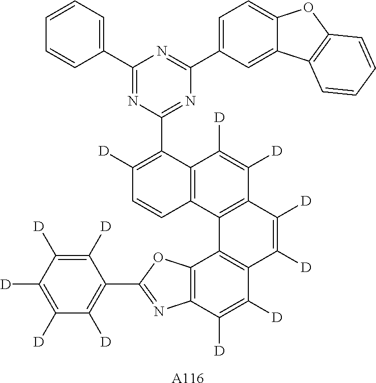 | 679.3 | 66 |
| 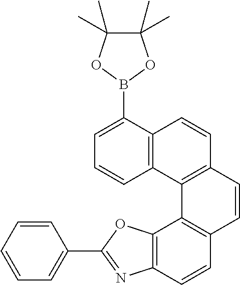 [SMILES: CC1(C)OB(c2cccc3c2ccc2ccc4ccc5nc(-c6ccccc6)oc5c4c23)OC1(C)C] | 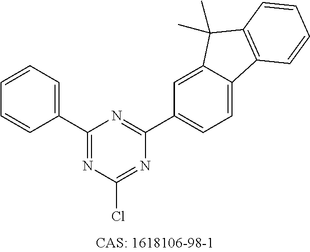 [SMILES: CC1(C)c2ccccc2-c2ccc(-c3nc(Cl)nc(-c4ccccc4)n3)cc21] | 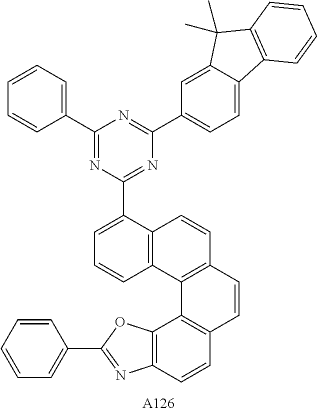 | 693.3 | 71 |
| 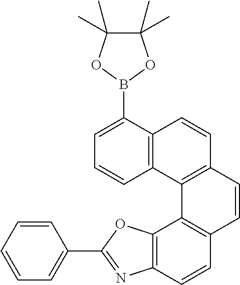 [SMILES: CC1(C)OB(c2cccc3c2ccc2ccc4ccc5nc(-c6ccccc6)oc5c4c23)OC1(C)C] | 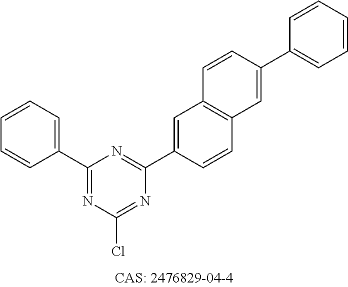 [SMILES: Clc1nc(-c2ccccc2)nc(-c2ccc3cc(-c4ccccc4)ccc3c2)n1] | 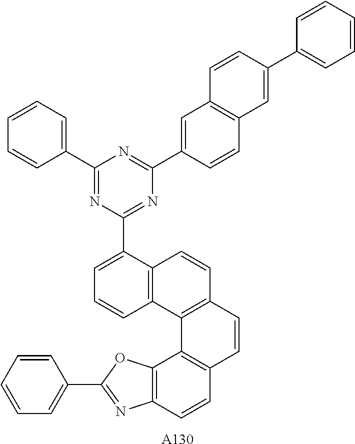 | 703.2 | 68 |
| 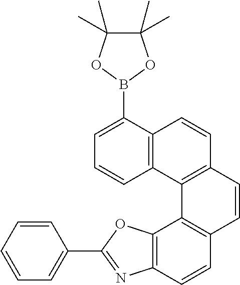 [SMILES: CC1(C)OB(c2cccc3c2ccc2ccc4ccc5nc(-c6ccccc6)oc5c4c23)OC1(C)C] | 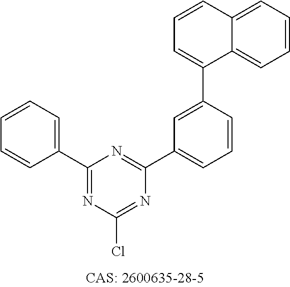 [SMILES: Clc1nc(-c2ccccc2)nc(-c2cccc(-c3cccc4ccccc34)c2)n1] | 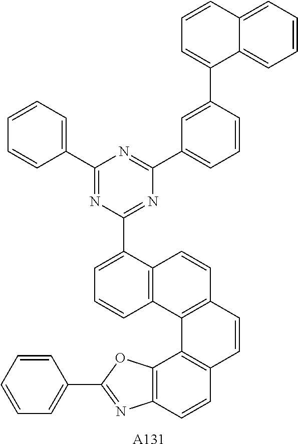 | 703.2 | 69 |
| 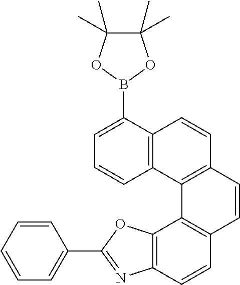 [SMILES: CC1(C)OB(c2cccc3c2ccc2ccc4ccc5nc(-c6ccccc6)oc5c4c23)OC1(C)C] | 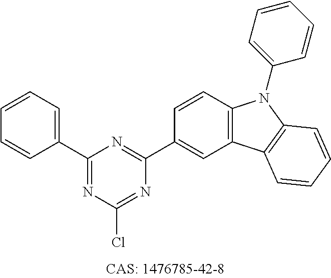 [SMILES: Clc1nc(-c2ccccc2)nc(-c2ccc3c(c2)c2ccccc2n3-c2ccccc2)n1] | 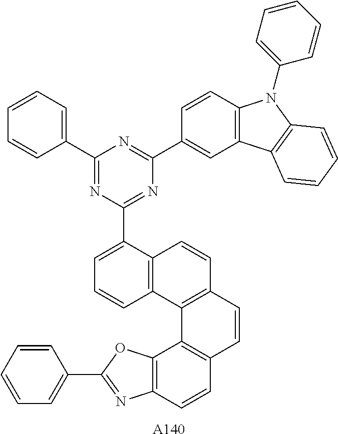 | 742.3 | 65 |
| 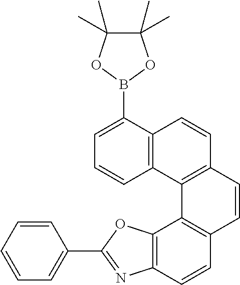 [SMILES: CC1(C)OB(c2cccc3c2ccc2ccc4ccc5nc(-c6ccccc6)oc5c4c23)OC1(C)C] | 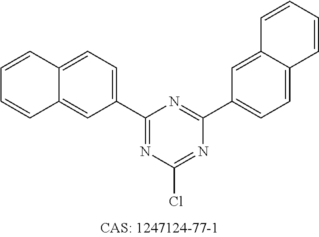 [SMILES: Clc1nc(-c2ccc3ccccc3c2)nc(-c2ccc3ccccc3c2)n1] | 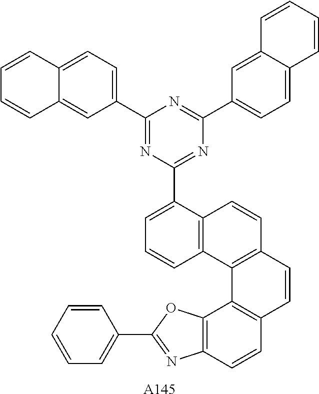 | 677.2 | 69 |
| 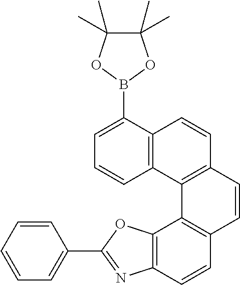 [SMILES: CC1(C)OB(c2cccc3c2ccc2ccc4ccc5nc(-c6ccccc6)oc5c4c23)OC1(C)C] | 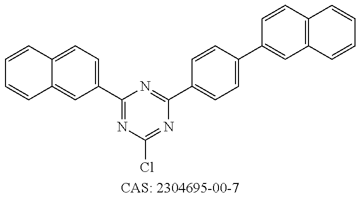 [SMILES: Clc1nc(-c2ccc(-c3ccc4ccccc4c3)cc2)nc(-c2ccc3ccccc3c2)n1] | 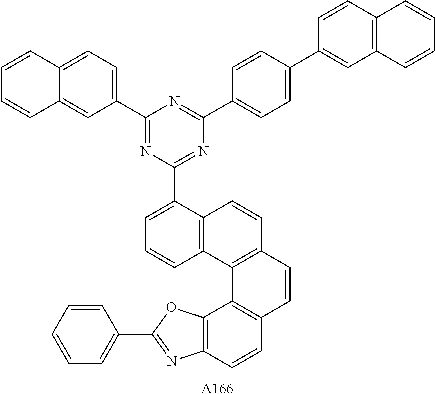 | 753.3 | 74 |
| 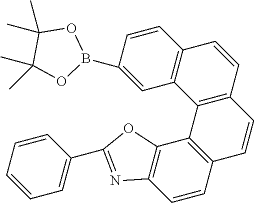 [SMILES: CC1(C)OB(c2ccc3ccc4ccc5ccc6nc(-c7ccccc7)oc6c5c4c3c2)OC1(C)C] | 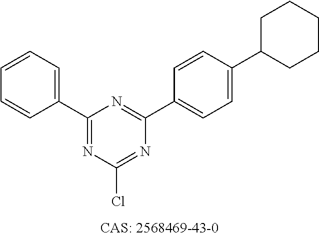 [SMILES: Clc1nc(-c2ccccc2)nc(-c2ccc(C3CCCCC3)cc2)n1] | 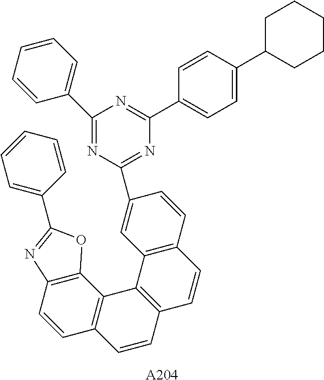 | 659.3 | 62 |
| 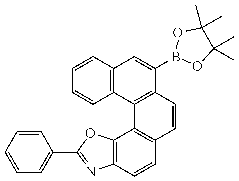 [SMILES: CC1(C)OB(c2cc3ccccc3c3c2ccc2ccc4nc(-c5ccccc5)oc4c23)OC1(C)C] | 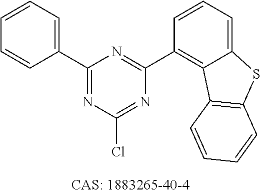 [SMILES: Clc1nc(-c2ccccc2)nc(-c2cccc3sc4ccccc4c23)n1] | 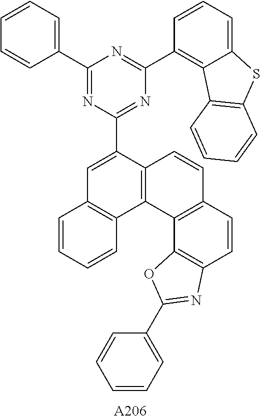 | 683.2 | 67 |
| 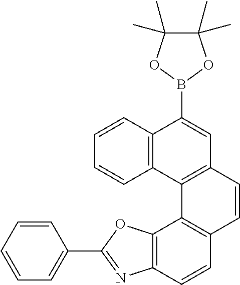 [SMILES: CC1(C)OB(c2cc3ccc4ccc5nc(-c6ccccc6)oc5c4c3c3ccccc23)OC1(C)C] | 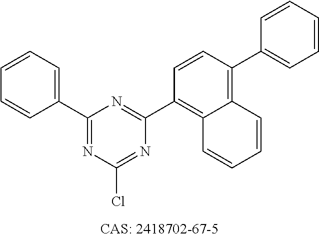 [SMILES: Clc1nc(-c2ccccc2)nc(-c2ccc(-c3ccccc3)c3ccccc23)n1] | 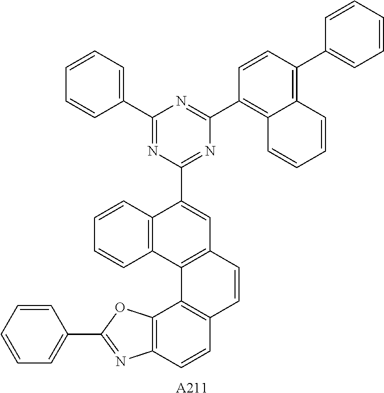 | 703.2 | 68 |
| 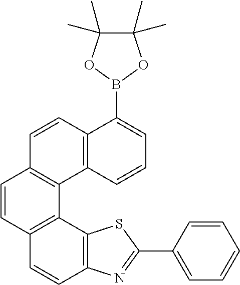 [SMILES: CC1(C)OB(c2cccc3c2ccc2ccc4ccc5nc(-c6ccccc6)sc5c4c23)OC1(C)C] | 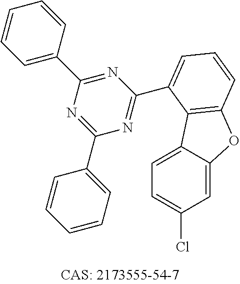 [SMILES: Clc1ccc2c(c1)oc1cccc(-c3nc(-c4ccccc4)nc(-c4ccccc4)n3)c12] | 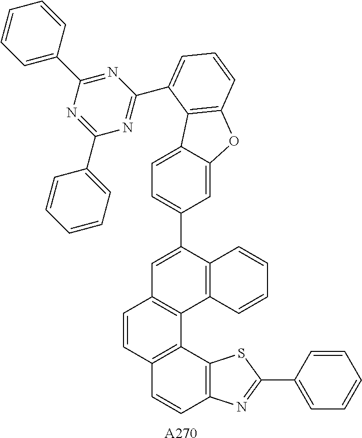 | 759.2 | 71 |
| 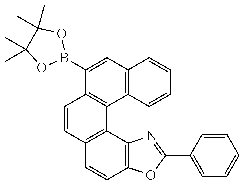 [SMILES: CC1(C)OB(c2cc3ccccc3c3c2ccc2ccc4oc(-c5ccccc5)nc4c23)OC1(C)C] | 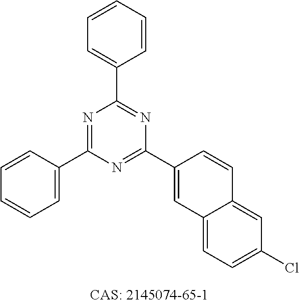 [SMILES: Clc1ccc2cc(-c3nc(-c4ccccc4)nc(-c4ccccc4)n3)ccc2c1] | 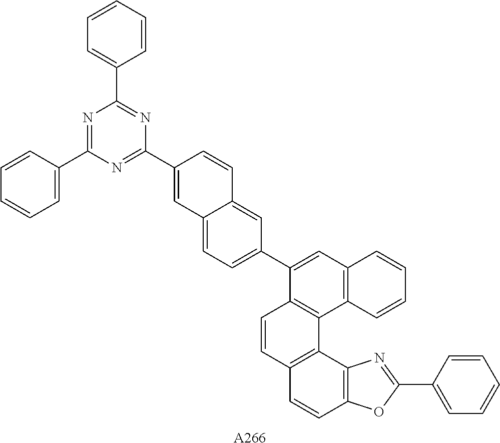 [SMILES: c1ccc(-c2nc(-c3ccccc3)nc(-c3ccc4cc(-c5cc6ccccc6c6c5ccc5ccc7oc(-c8ccccc8)nc7c56)ccc4c3)n2)cc1 ; "A266"] | 703.2 | 66 |
| 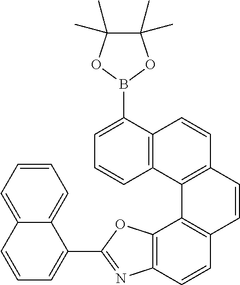 [SMILES: CC1(C)OB(c2cccc3c2ccc2ccc4ccc5nc(-c6cccc7ccccc67)oc5c4c23)OC1(C)C] | 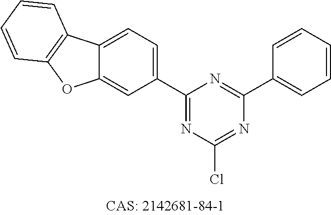 [SMILES: Clc1nc(-c2ccccc2)nc(-c2ccc3c(c2)oc2ccccc23)n1] | 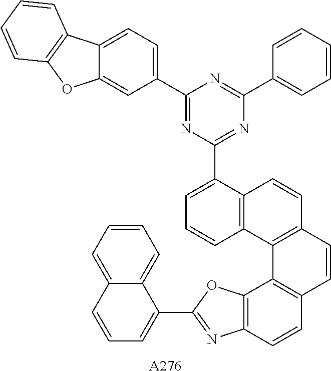 | 717.2 | 75 |
| 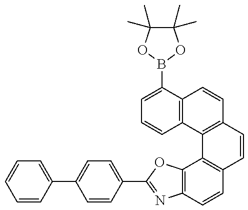 [SMILES: CC1(C)OB(c2cccc3c2ccc2ccc4ccc5nc(-c6ccc(-c7ccccc7)cc6)oc5c4c23)OC1(C)C] | 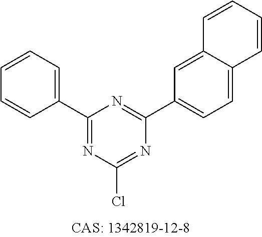 [SMILES: Clc1nc(-c2ccccc2)nc(-c2ccc3ccccc3c2)n1] | 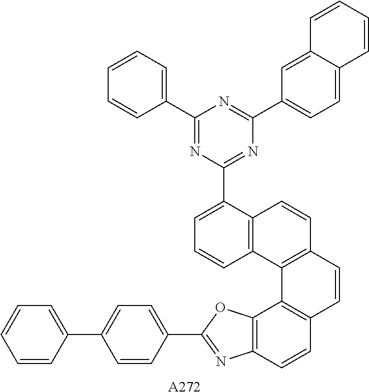 | 703.2 | 65 |
| 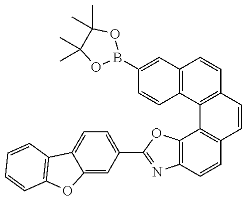 [SMILES: CC1(C)OB(c2ccc3c(ccc4ccc5ccc6nc(-c7ccc8c(c7)oc7ccccc78)oc6c5c43)c2)OC1(C)C] | 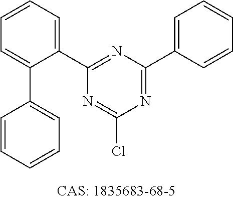 [SMILES: Clc1nc(-c2ccccc2)nc(-c2ccccc2-c2ccccc2)n1] | 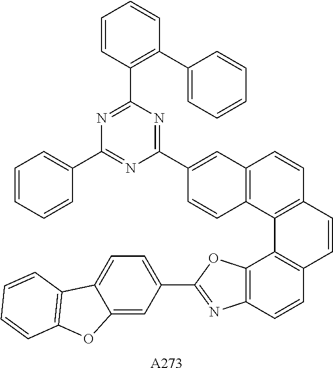 [SMILES: c1ccc(-c2nc(-c3ccc4c(ccc5ccc6ccc7nc(-c8ccc9c(c8)oc8ccccc89)oc7c6c54)c3)nc(-c3ccccc3-c3ccccc3)n2)cc1 ; "A273"] | 743.2 | 73 |
| 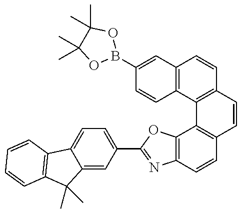 [SMILES: CC1(C)c2ccccc2-c2ccc(-c3nc4ccc5ccc6ccc7cc(B8OC(C)(C)C(C)(C)O8)ccc7c6c5c4o3)cc21] | 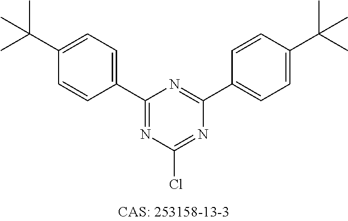 [SMILES: CC(C)(C)c1ccc(-c2nc(Cl)nc(-c3ccc(C(C)(C)C)cc3)n2)cc1] | 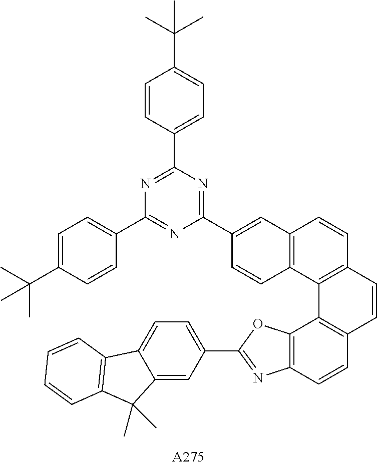 | 805.4 | 75 |

### Synthesis of Compound B6:

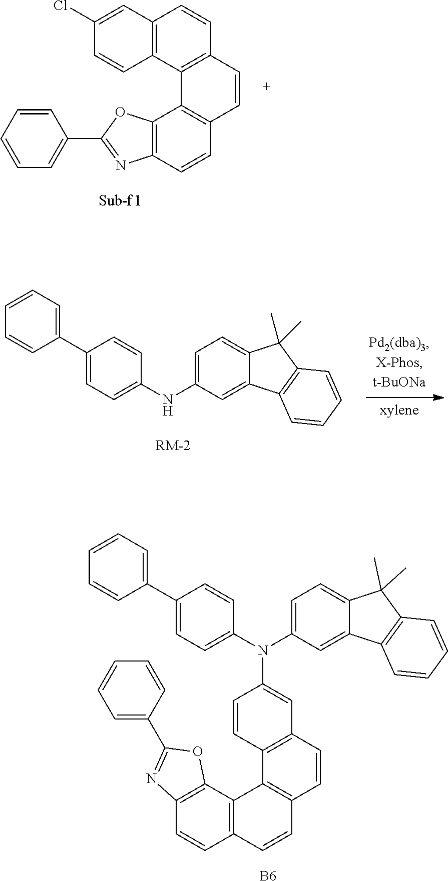

Under a nitrogen atmosphere, to a 500 mL three-necked flask was added Sub-f1 (9.50 g, 25 mmol), RM-2 (CAS:1326137-97-6, 9.04 g, 25 mmol), tris (dibenzylideneacetone) dipalladium (0.916 g, 0.5 mmol), (2-dicyclohexylphosphine-2′, 4′, 6′-triisopropylbiphenyl) (0.95 g, 1 mmol), sodium tert-butoxide (9.61 g, 50 mmol) and xylene (250 mL) in turn. The resulting mixture was heated to reflux and stirred overnight. After the reaction solution was cooled to room temperature, it was extracted with dichloromethane (100 mL×3 times), and the organic phases were combined and dried with anhydrous sodium sulfate, followed by filtration and then reduced pressure distillation to remove the solvent, to get a crude product. The crude product was purified by silica gel column chromatography with a mixture of n-heptane and dichloromethane as the mobile phase to obtain an off-white solid Compound B6 (14.45 g, yield 82%, m/z=705.3 [M+H]⁺).

Referring to the synthesis of Compound B6, the compounds of the present disclosure in Table 10 were synthesized by replacing Sub-f1 with Reactant 0, and replacing RM-2 with Reactant P as shown in Table 10.

TABLE 10
Synthesis of the compounds of the present disclosure

| | | | m/z | |
|---|---|---|---|---|
| | | Compound structure | ([M + | |
| Reactant O | Reactant P | and number | H]⁺) | Yield % |
| 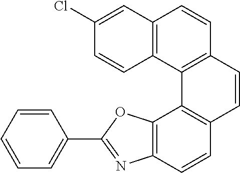 [SMILES: Clc1ccc2c(ccc3ccc4ccc5nc(-c6ccccc6)oc5c4c32)c1] | 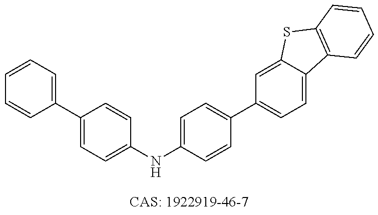 [SMILES: c1ccc(-c2ccc(Nc3ccc(-c4ccc5c(c4)sc4ccccc45)cc3)cc2)cc1] | 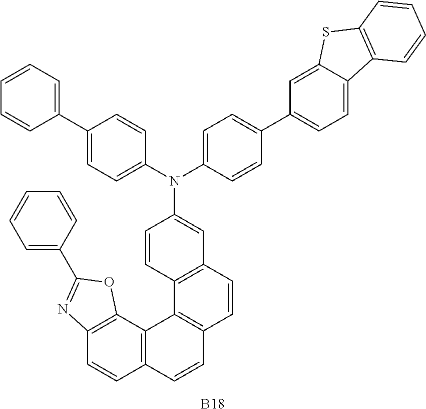 | 771.2 | 76 |
| 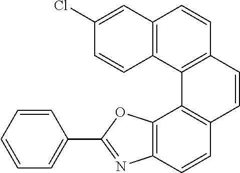 [SMILES: Clc1ccc2c(ccc3ccc4ccc5nc(-c6ccccc6)oc5c4c32)c1] | 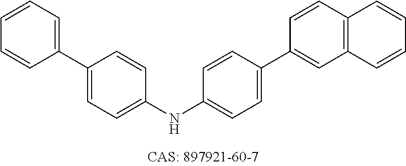 [SMILES: c1ccc(-c2ccc(Nc3ccc(-c4ccc5ccccc5c4)cc3)cc2)cc1] | 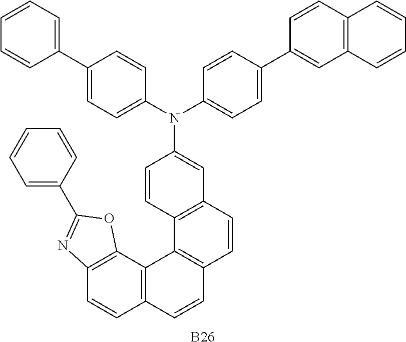 | 715.3 | 70 |
| 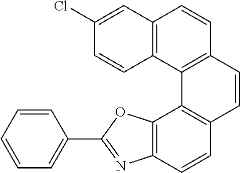 [SMILES: Clc1ccc2c(ccc3ccc4ccc5nc(-c6ccccc6)oc5c4c32)c1] | 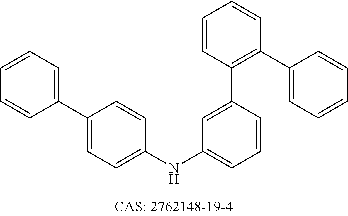 [SMILES: c1ccc(-c2ccc(Nc3cccc(-c4ccccc4-c4ccccc4)c3)cc2)cc1] | 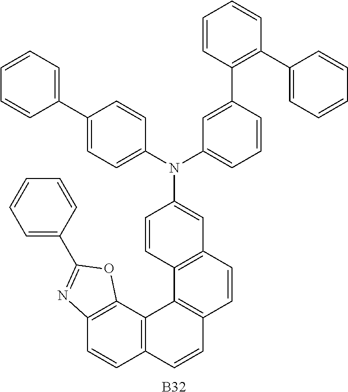 | 741.3 | 79 |
| 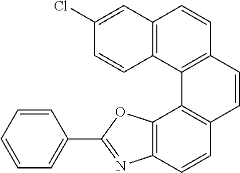 [SMILES: Clc1ccc2c(ccc3ccc4ccc5nc(-c6ccccc6)oc5c4c32)c1] | 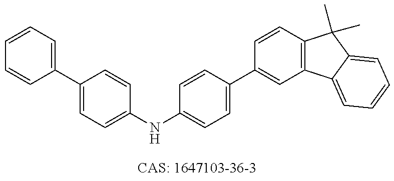 [SMILES: CC1(C)c2ccccc2-c2cc(-c3ccc(Nc4ccc(-c5ccccc5)cc4)cc3)ccc21] | 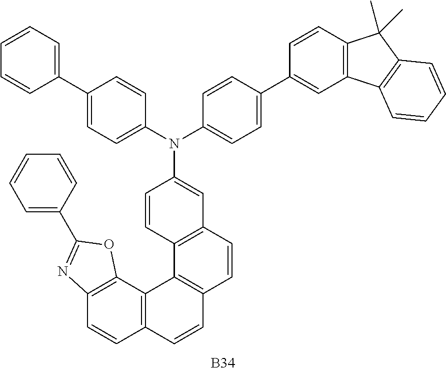 | 781.3 | 77 |
| 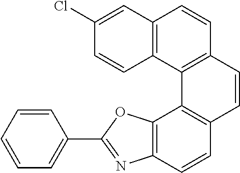 [SMILES: Clc1ccc2c(ccc3ccc4ccc5nc(-c6ccccc6)oc5c4c32)c1] | 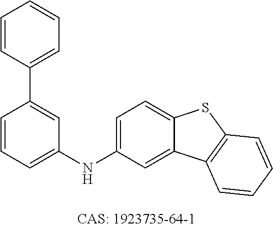 [SMILES: c1ccc(-c2cccc(Nc3ccc4sc5ccccc5c4c3)c2)cc1] | 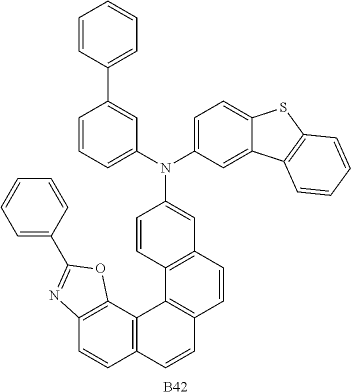 | 695.2 | 80 |
| 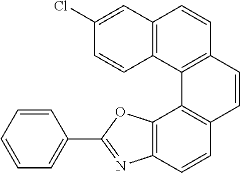 [SMILES: Clc1ccc2c(ccc3ccc4ccc5nc(-c6ccccc6)oc5c4c32)c1] | 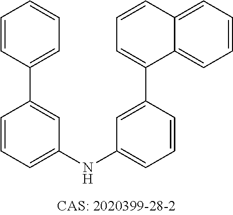 [SMILES: c1ccc(-c2cccc(Nc3cccc(-c4cccc5ccccc45)c3)c2)cc1] | 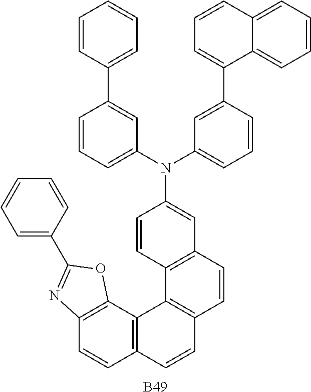 | 715.3 | 78 |
| 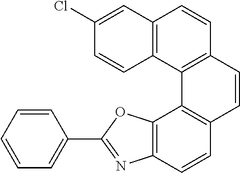 [SMILES: Clc1ccc2c(ccc3ccc4ccc5nc(-c6ccccc6)oc5c4c32)c1] | 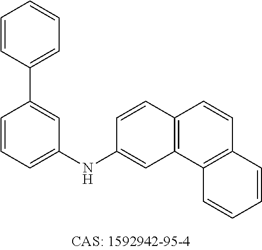 [SMILES: c1ccc(-c2cccc(Nc3ccc4ccc5ccccc5c4c3)c2)cc1] | 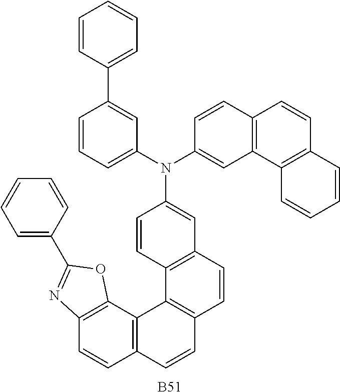 | 689.2 | 70 |
| 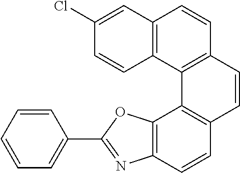 [SMILES: Clc1ccc2c(ccc3ccc4ccc5nc(-c6ccccc6)oc5c4c32)c1] | 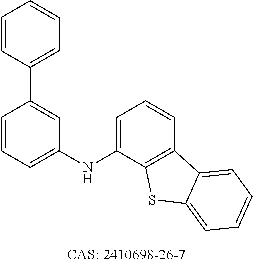 [SMILES: c1ccc(-c2cccc(Nc3cccc4c3sc3ccccc34)c2)cc1] | 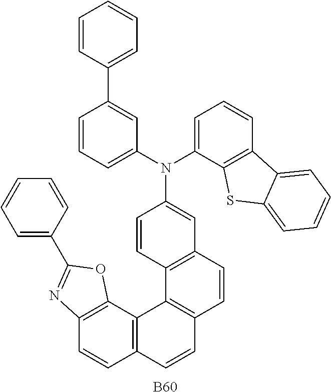 | 695.2 | 78 |
| 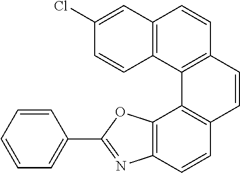 [SMILES: Clc1ccc2c(ccc3ccc4ccc5nc(-c6ccccc6)oc5c4c32)c1] | 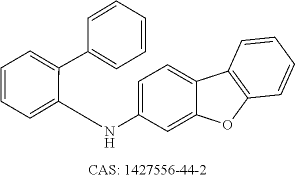 [SMILES: c1ccc(-c2ccccc2Nc2ccc3c(c2)oc2ccccc23)cc1] | 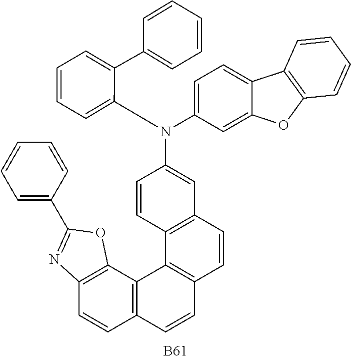 | 679.2 | 70 |
| 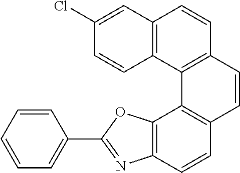 [SMILES: Clc1ccc2c(ccc3ccc4ccc5nc(-c6ccccc6)oc5c4c32)c1] | 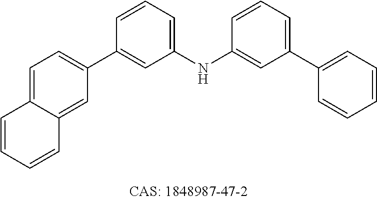 [SMILES: c1ccc(-c2cccc(Nc3cccc(-c4ccc5ccccc5c4)c3)c2)cc1] | 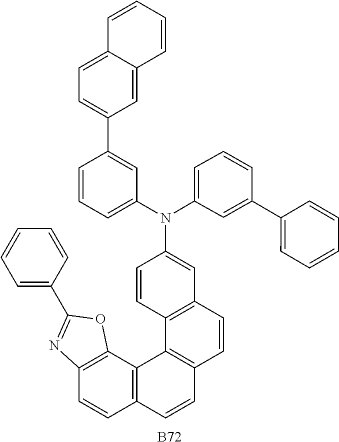 | 715.3 | 80 |
| 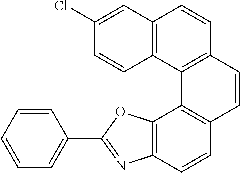 [SMILES: Clc1ccc2c(ccc3ccc4ccc5nc(-c6ccccc6)oc5c4c32)c1] | 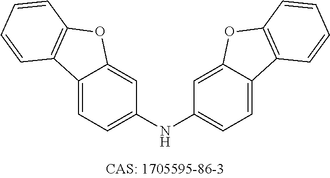 [SMILES: c1ccc2c(c1)oc1cc(Nc3ccc4c(c3)oc3ccccc34)ccc12] | 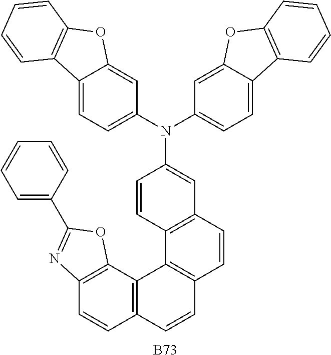 [SMILES: "B73" ; c1ccc(-c2nc3ccc4ccc5ccc6cc(N(c7ccc8c(c7)oc7ccccc78)c7ccc8c(c7)oc7ccccc78)ccc6c5c4c3o2)cc1] | 693.2 | 80 |
| 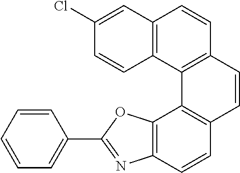 [SMILES: Clc1ccc2c(ccc3ccc4ccc5nc(-c6ccccc6)oc5c4c32)c1] | 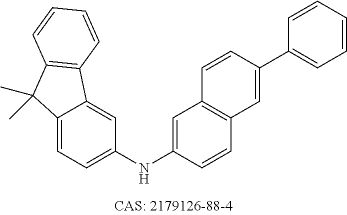 [SMILES: CC1(C)c2ccccc2-c2cc(Nc3ccc4cc(-c5ccccc5)ccc4c3)ccc21] | 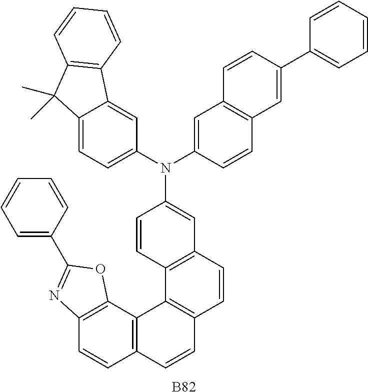 [SMILES: "B82" ; CC1(C)c2ccccc2-c2cc(N(c3ccc4cc(-c5ccccc5)ccc4c3)c3ccc4c(ccc5ccc6ccc7nc(-c8ccccc8)oc7c6c54)c3)ccc21] | 755.3 | 75 |
| 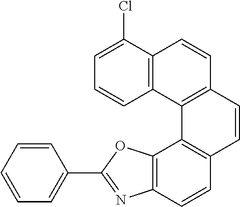 [SMILES: Clc1cccc2c1ccc1ccc3ccc4nc(-c5ccccc5)oc4c3c12] | 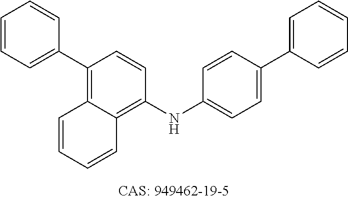 [SMILES: c1ccc(-c2ccc(Nc3ccc(-c4ccccc4)c4ccccc34)cc2)cc1] | 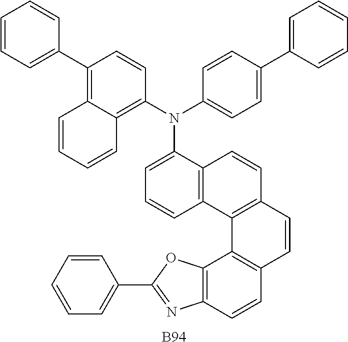 [SMILES: "B94" ; c1ccc(-c2ccc(N(c3ccc(-c4ccccc4)c4ccccc34)c3cccc4c3ccc3ccc5ccc6nc(-c7ccccc7)oc6c5c34)cc2)cc1] | 715.3 | 68 |
| 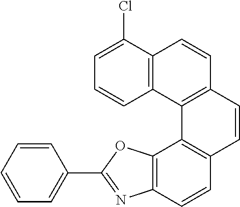 [SMILES: Clc1cccc2c1ccc1ccc3ccc4nc(-c5ccccc5)oc4c3c12] | 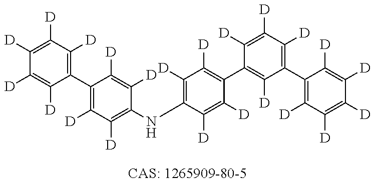 [SMILES: [2H]c1c([2H])c([2H])c(-c2c([2H])c([2H])c(Nc3c([2H])c([2H])c(-c4c([2H])c([2H])c([2H])c(-c5c([2H])c([2H])c([2H])c([2H])c5[2H])c4[2H])c([2H])c3[2H])c([2H])c2[2H])c([2H])c1[2H]] | 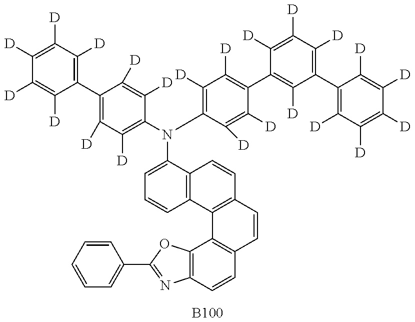 | 761.4 | 74 |
| 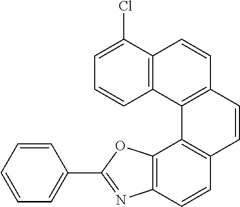 [SMILES: Clc1cccc2c1ccc1ccc3ccc4nc(-c5ccccc5)oc4c3c12] | 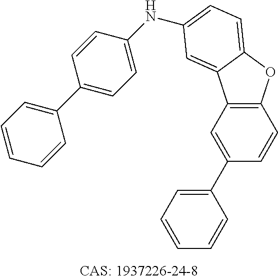 [SMILES: c1ccc(-c2ccc(Nc3ccc4oc5ccc(-c6ccccc6)cc5c4c3)cc2)cc1] | 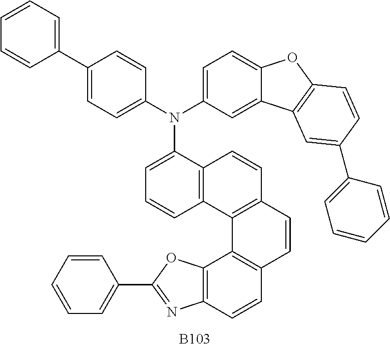 [SMILES: "B103" ; c1ccc(-c2ccc(N(c3ccc4oc5ccc(-c6ccccc6)cc5c4c3)c3cccc4c3ccc3ccc5ccc6nc(-c7ccccc7)oc6c5c34)cc2)cc1] | 755.3 | 79 |
| 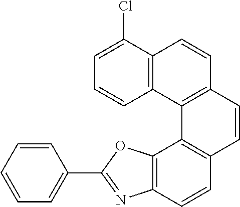 [SMILES: Clc1cccc2c1ccc1ccc3ccc4nc(-c5ccccc5)oc4c3c12] | 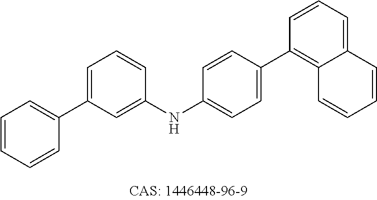 [SMILES: c1ccc(-c2cccc(Nc3ccc(-c4cccc5ccccc45)cc3)c2)cc1] | 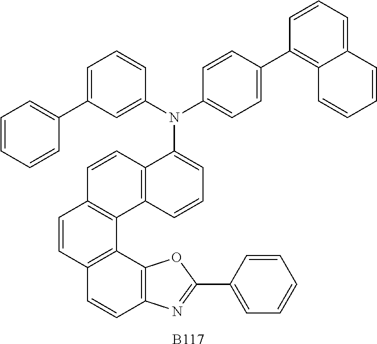 [SMILES: "B117" ; c1ccc(-c2cccc(N(c3ccc(-c4cccc5ccccc45)cc3)c3cccc4c3ccc3ccc5ccc6nc(-c7ccccc7)oc6c5c34)c2)cc1] | 715.3 | 73 |
| 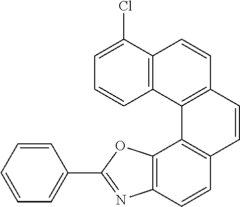 [SMILES: Clc1cccc2c1ccc1ccc3ccc4nc(-c5ccccc5)oc4c3c12] | 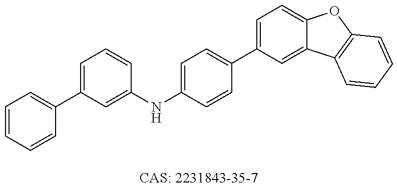 [SMILES: c1ccc(-c2cccc(Nc3ccc(-c4ccc5oc6ccccc6c5c4)cc3)c2)cc1] | 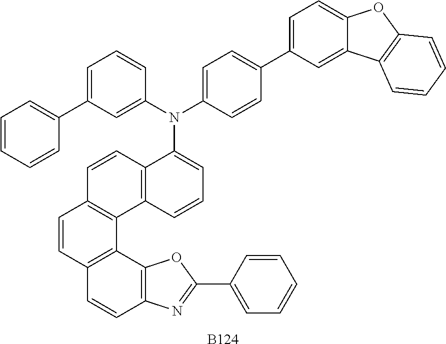 | 755.3 | 76 |
| 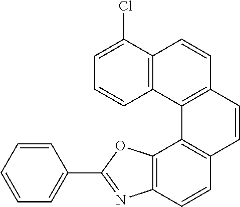 [SMILES: Clc1cccc2c1ccc1ccc3ccc4nc(-c5ccccc5)oc4c3c12] | 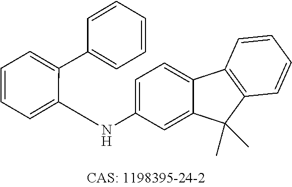 [SMILES: CC1(C)c2ccccc2-c2ccc(Nc3ccccc3-c3ccccc3)cc21] | 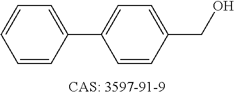 [SMILES: OCc1ccc(-c2ccccc2)cc1] | 705.3 | 72 |
| 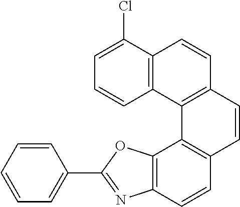 [SMILES: Clc1cccc2c1ccc1ccc3ccc4nc(-c5ccccc5)oc4c3c12] | 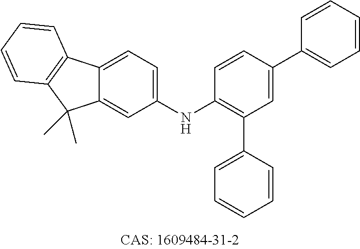 [SMILES: CC1(C)c2ccccc2-c2ccc(Nc3ccc(-c4ccccc4)cc3-c3ccccc3)cc21] | 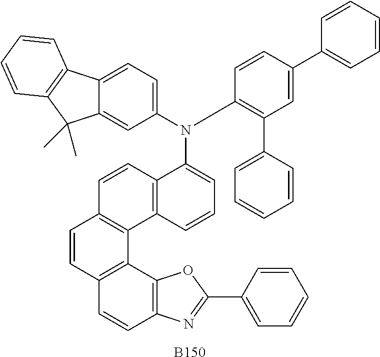 [SMILES: "B150" ; CC1(C)c2ccccc2-c2ccc(N(c3ccc(-c4ccccc4)cc3-c3ccccc3)c3cccc4c3ccc3ccc5ccc6nc(-c7ccccc7)oc6c5c34)cc21] | 781.3 | 65 |
| 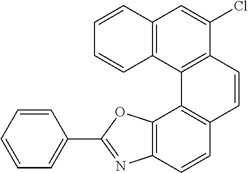 [SMILES: Clc1cc2ccccc2c2c1ccc1ccc3nc(-c4ccccc4)oc3c12] | 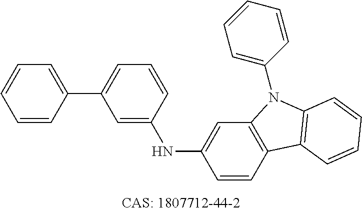 [SMILES: c1ccc(-c2cccc(Nc3ccc4c5ccccc5n(-c5ccccc5)c4c3)c2)cc1] | 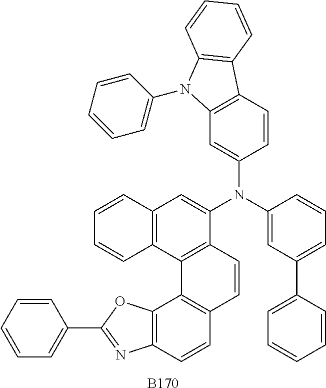 [SMILES: "B170" ; c1ccc(-c2cccc(N(c3ccc4c5ccccc5n(-c5ccccc5)c4c3)c3cc4ccccc4c4c3ccc3ccc5nc(-c6ccccc6)oc5c34)c2)cc1] | 754.3 | 70 |
| 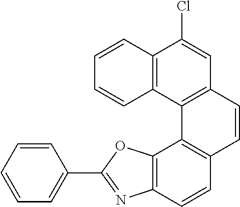 [SMILES: Clc1cc2ccc3ccc4nc(-c5ccccc5)oc4c3c2c2ccccc12] | 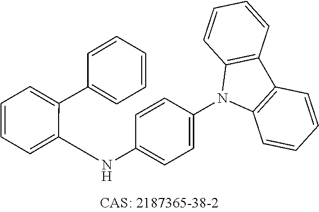 [SMILES: c1ccc(-c2ccccc2Nc2ccc(-n3c4ccccc4c4ccccc43)cc2)cc1] | 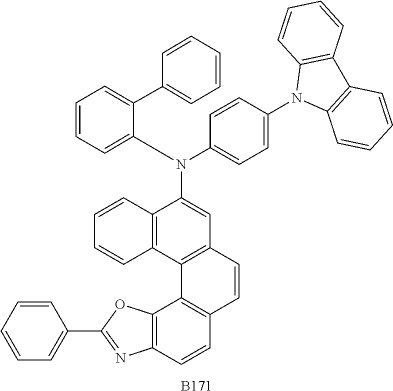 [SMILES: "B171" ; c1ccc(-c2nc3ccc4ccc5cc(N(c6ccc(-n7c8ccccc8c8ccccc87)cc6)c6ccccc6-c6ccccc6)c6ccccc6c5c4c3o2)cc1] | 754.3 | 70 |
| 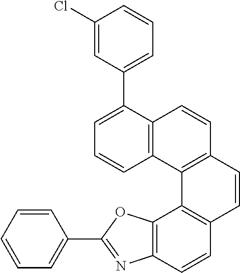 [SMILES: Clc1cccc(-c2cccc3c2ccc2ccc4ccc5nc(-c6ccccc6)oc5c4c23)c1] | 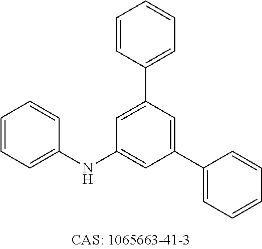 [SMILES: c1ccc(Nc2cc(-c3ccccc3)cc(-c3ccccc3)c2)cc1] | 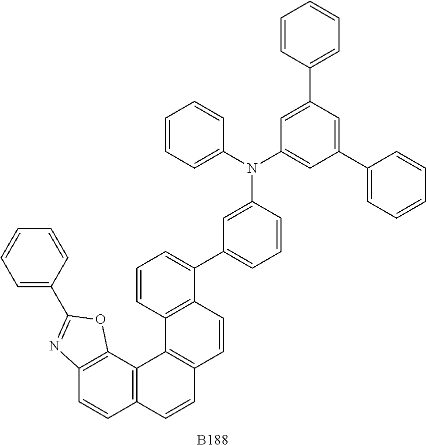 [SMILES: "B188" ; c1ccc(-c2cc(-c3ccccc3)cc(N(c3ccccc3)c3cccc(-c4cccc5c4ccc4ccc6ccc7nc(-c8ccccc8)oc7c6c45)c3)c2)cc1] | 741.3 | 72 |
| 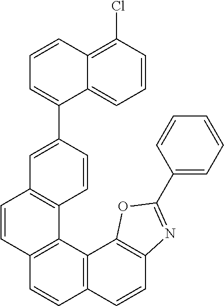 [SMILES: Clc1cccc2c(-c3ccc4c(ccc5ccc6ccc7nc(-c8ccccc8)oc7c6c54)c3)cccc12] | 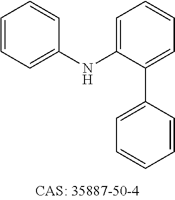 [SMILES: c1ccc(Nc2ccccc2-c2ccccc2)cc1] | 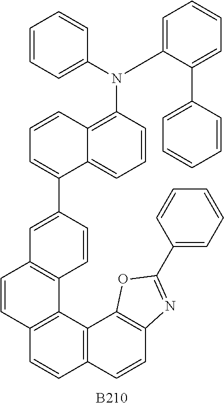 [SMILES: "B210" ; c1ccc(-c2nc3ccc4ccc5ccc6cc(-c7cccc8c(N(c9ccccc9)c9ccccc9-c9ccccc9)cccc78)ccc6c5c4c3o2)cc1] | 715.3 | 69 |
| 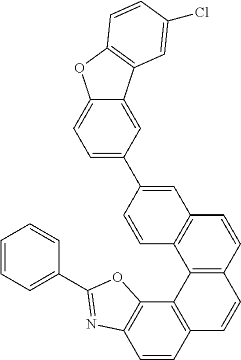 [SMILES: Clc1ccc2oc3ccc(-c4ccc5c(ccc6ccc7ccc8nc(-c9ccccc9)oc8c7c65)c4)cc3c2c1] | 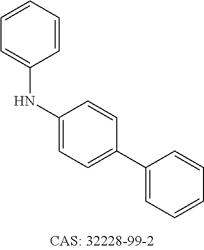 [SMILES: c1ccc(Nc2ccc(-c3ccccc3)cc2)cc1] | 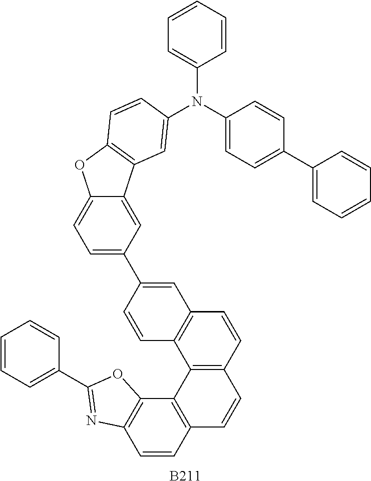 [SMILES: "B211" ; c1ccc(-c2ccc(N(c3ccccc3)c3ccc4oc5ccc(-c6ccc7c(ccc8ccc9ccc%10nc(-c%11ccccc%11)oc%10c9c87)c6)cc5c4c3)cc2)cc1] | 755.3 | 67 |
| 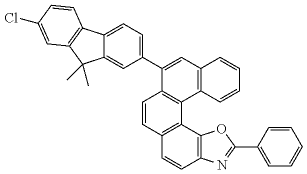 [SMILES: CC1(C)c2cc(Cl)ccc2-c2ccc(-c3cc4ccccc4c4c3ccc3ccc5nc(-c6ccccc6)oc5c34)cc21] | 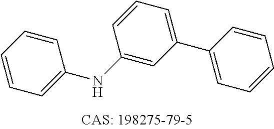 [SMILES: c1ccc(Nc2cccc(-c3ccccc3)c2)cc1] | 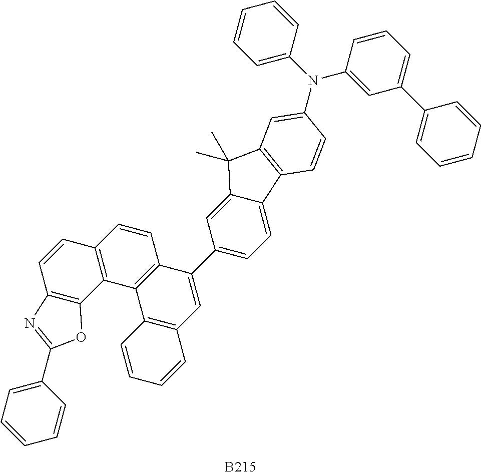 [SMILES: "B215" ; CC1(C)c2cc(-c3cc4ccccc4c4c3ccc3ccc5nc(-c6ccccc6)oc5c34)ccc2-c2ccc(N(c3ccccc3)c3cccc(-c4ccccc4)c3)cc21] | 781.3 | 66 |
| 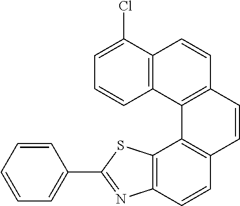 [SMILES: Clc1cccc2c1ccc1ccc3ccc4nc(-c5ccccc5)sc4c3c12] | 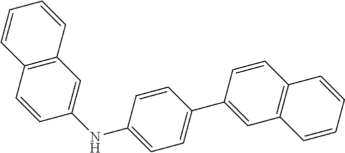 [SMILES: c1ccc2cc(Nc3ccc(-c4ccc5ccccc5c4)cc3)ccc2c1] | 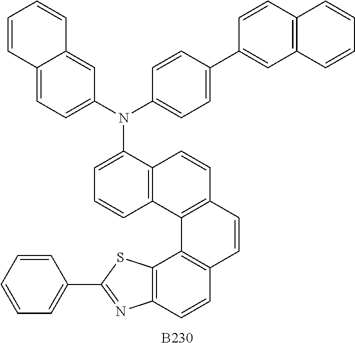 [SMILES: "B230" ; c1ccc(-c2nc3ccc4ccc5ccc6c(N(c7ccc(-c8ccc9ccccc9c8)cc7)c7ccc8ccccc8c7)cccc6c5c4c3s2)cc1] | 705.2 | 60 |

NMR data of Compound A145: ¹H-NMR (400 MHz, CD₂Cl₂) 6 ppm: 8.84 (s, 2H), 8.65 (d, 1H), 8.61 (d, 1H), 8.55-8.50 (m, 3H), 8.35 (d, 2H), 8.18-8.05 (m, 8H), 8.02-7.96 (m, 3H), 7.83 (t, 1H), 7.64 (t, 2H), 7.57 (t, 2H), 7.49 (t, 2H), 7.38 (t, 1H).

NMR data of Compound B61: ¹H-NMR (400 MHz, CD₂Cl₂) 6 ppm: 8.35 (d, 2H), 8.17 (d, 1H), 8.08 (d, 1H), 8.00-7.89 (m, 5H), 7.67 (d, 1H), 7.59-7.53 (m, 3H), 7.49-7.32 (m, 9H), 7.27-7.15 (m, 4H), 6.99 (s, 1H), 6.80 (d, 1H), 6.73 (d, 1H), 6.66 (d, 1H).

### Fabrication and Evaluation of Organic Electroluminescent Device

### Example 1: Fabrication of the Red Organic Electroluminescent Device

First, anode pretreatment was performed by the following processes. The surface of a ITO/Ag/ITO substrate with a thickness of 100 Å, 1000 Å, and 100 Å respectively, was treated using ultraviolet ozone and O₂:N₂ plasma to increase the work function of the anode. The surface of the ITO substrate can also be cleaned with an organic solvent to remove impurities and oil thereon.

On the experimental substrate (anode), PD and α-NPD were co-evaporated at an evaporation ratio of 2%:98% to form a hole injection layer (HIL) with a thickness of 100 Å, and then α-NPD was vacuum evaporated on the hole injection layer to form a hole transport layer with a thickness of 1055 Å. The compound HT-1 was vacuum evaporated on the first hole transport layer to form a second hole transport layer with a thickness of 890 Å.

Then, on the second hole transport layer, the compound A3:RH-P:RD were co-evaporated at a ratio of 49%:49%:2% to form a red light-emitting layer (EML) with a thickness of 400 Å.

On the light-emitting layer, the compounds ET-1 and LiQ were co-evaporated at an evaporation rate ratio of 1:1 to form an electron transport layer (ETL) with a thickness of 350 Å, and Yb was evaporated on the electron transport layer to form an electron injection layer (EIL) with a thickness of 10 Å. Then, magnesium (Mg) and silver (Ag) were mixed at a evaporation rate of 1:9 and vacuum evaporated on the electron injection layer to form a cathode with a thickness of 130 Å.

In addition, CP with a thickness of 800 Å was vacuum evaporated on the above cathode to complete the manufacture of the red organic electroluminescent device.

### Examples 2 to 33

Organic electroluminescent devices were fabricated by the same method as used in Example 1, except that Compound A3 in Example 1 was replaced with one of the compounds in the following Table 11 when an light-emitting layer was formed.

### Comparative Examples 1 to 3

Organic electroluminescent devices were fabricated by the same method as used in Example 1, except that Compound A3 in Example 1 was replaced with Compound A, Compound B, or Compound C when an light-emitting layer was formed.

The organic electroluminescent devices fabricated in the Examples 1 to 33 and Comparative Examples 1 to 3 were tested for their performance. Specifically, the IVL characteristics of the devices were tested under the condition of 10 mA/cm², and the T95 lifetime of the devices were tested under the condition of 20 mA/cm². Test results are shown in Table 11.

Structures of the main materials used in the Examples and Comparative Examples are shown in below.

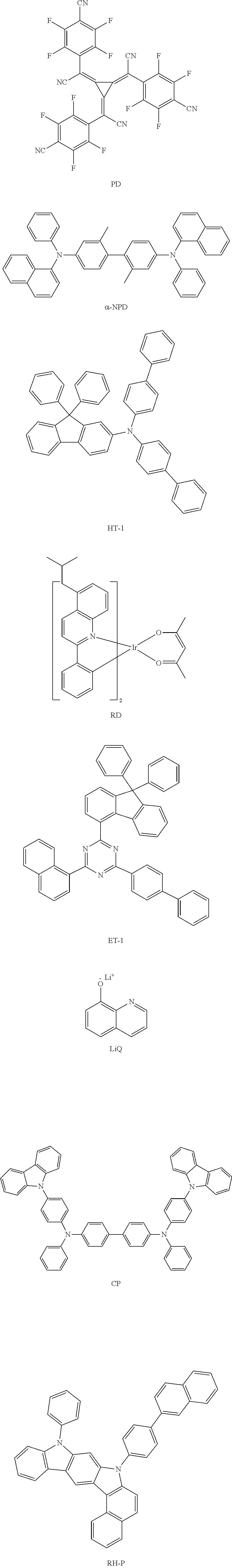

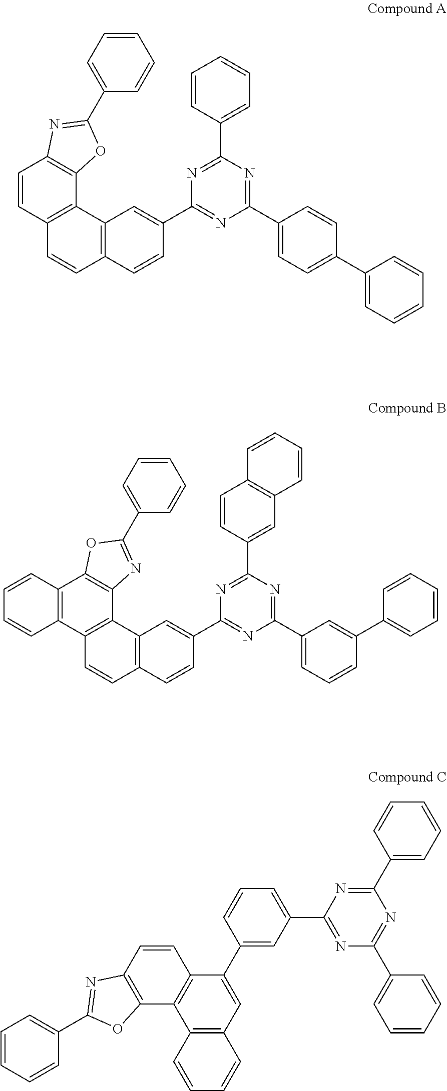 [SMILES: c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2cccc(-c3nc(-c4ccc5ccccc5c4)nc(-c4ccc5ccc6c7ccccc7c7oc(-c8ccccc8)nc7c6c5c4)n3)c2)cc1 ; c1ccc(-c2nc(-c3ccccc3)nc(-c3cccc(-c4cc5ccccc5c5c4ccc4nc(-c6ccccc6)oc45)c3)n2)cc1]

TABLE 11

| | Light-emitting | | | | | |
|---|---|---|---|---|---|---|
| | layer | Operating | | | | |
| Example | Compound X: | voltage | | | | T95(hrs) |
| number | RH-P:RD | Volt (V) | Cd/A | CIEx | CIEy | @20 mA/cm² |
| Example 1 | Compound A3 | 3.48 | 59.2 | 0.680 | 0.320 | 525 |
| Example 2 | Compound A4 | 3.45 | 59.4 | 0.680 | 0.320 | 491 |
| Example 3 | Compound A8 | 3.48 | 61.4 | 0.680 | 0.320 | 511 |
| Example 4 | Compound A10 | 3.48 | 60.2 | 0.680 | 0.320 | 492 |
| Example 5 | Compound A17 | 3.47 | 59.5 | 0.680 | 0.320 | 493 |
| Example 6 | Compound A19 | 3.46 | 60.3 | 0.680 | 0.320 | 505 |
| Example 7 | Compound A23 | 3.45 | 59.3 | 0.680 | 0.320 | 495 |
| Example 8 | Compound A25 | 3.46 | 60.1 | 0.680 | 0.320 | 501 |
| Example 9 | Compound A39 | 3.50 | 59.7 | 0.680 | 0.320 | 501 |
| Example 10 | Compound A40 | 3.48 | 61.1 | 0.680 | 0.320 | 497 |
| Example 11 | Compound A45 | 3.49 | 59.1 | 0.680 | 0.320 | 503 |
| Example 12 | Compound A51 | 3.47 | 59.7 | 0.680 | 0.320 | 499 |
| Example 13 | Compound A61 | 3.50 | 59.4 | 0.680 | 0.320 | 496 |
| Example 14 | Compound A77 | 3.46 | 59.6 | 0.680 | 0.320 | 509 |
| Example 15 | Compound A83 | 3.45 | 59.5 | 0.680 | 0.320 | 512 |
| Example 16 | Compound A89 | 3.46 | 59.9 | 0.680 | 0.320 | 506 |
| Example 17 | Compound A114 | 3.45 | 61.7 | 0.680 | 0.320 | 503 |
| Example 18 | Compound A116 | 3.45 | 60.5 | 0.680 | 0.320 | 531 |
| Example 19 | Compound A126 | 3.46 | 59.7 | 0.680 | 0.320 | 508 |
| Example 20 | Compound A130 | 3.50 | 61.4 | 0.680 | 0.320 | 506 |
| Example 21 | Compound A131 | 3.45 | 59.3 | 0.680 | 0.320 | 503 |
| Example 22 | Compound A140 | 3.50 | 60.5 | 0.680 | 0.320 | 491 |
| Example 23 | Compound A145 | 3.48 | 61.6 | 0.680 | 0.320 | 512 |
| Example 24 | Compound A166 | 3.46 | 61.7 | 0.680 | 0.320 | 507 |
| Example 25 | Compound A204 | 3.48 | 54.4 | 0.680 | 0.320 | 495 |
| Example 26 | Compound A206 | 3.50 | 53.1 | 0.680 | 0.320 | 508 |
| Example 27 | Compound A211 | 3.45 | 54.2 | 0.680 | 0.320 | 512 |
| Example 28 | Compound A270 | 3.49 | 53.6 | 0.680 | 0.320 | 484 |
| Example 29 | Compound A266 | 3.45 | 53.8 | 0.680 | 0.320 | 484 |
| Example 30 | Compound A276 | 3.49 | 60.7 | 0.680 | 0.320 | 487 |
| Example 31 | Compound A272 | 3.46 | 61.4 | 0.680 | 0.320 | 480 |
| Example 32 | Compound A273 | 3.50 | 53.5 | 0.680 | 0.320 | 509 |
| Example 33 | Compound A275 | 3.50 | 60.6 | 0.680 | 0.320 | 503 |
| Comparative | Compound A | 3.51 | 47.2 | 0.680 | 0.320 | 396 |
| Example 1 | | | | | | |
| Comparative | Compound B | 3.52 | 48.1 | 0.680 | 0.320 | 415 |
| Example 2 | | | | | | |
| Comparative | Compound C | 3.67 | 46.6 | 0.680 | 0.320 | 380 |
| Example 3 | | | | | | |

According to Table 11 above, when the compound of the present disclosure is used as an electron-transporting host material in the host material of the light-emitting layer of the red organic electroluminescent device, the luminous efficiency of the device is improved by at least 10.4% and the lifetime of the device is improved by at least 15.7%.

### Example 34: The Red Organic Electroluminescent Device

First, anode pretreatment was performed by the following processes. The surface of ITO/Ag/ITO substrate with a thickness of 100 Å, 1000 Å, and 100 Å respectively was treated using ultraviolet ozone and O₂:N₂ plasma to increase the work function of the anode. The surface of the ITO substrate can also be cleaned with an organic solvent to remove impurities and oil thereon.

On the experimental substrate (anode), PD and α-NPD were co-evaporated at an evaporation ratio of 2%:98% to form a hole injection layer (HIL) with a thickness of 100 Å, and then α-NPD was vacuum evaporated on the hole injection layer to form a hole transport layer with a thickness of 1065 Å. The compound HT-1 was vacuum evaporated on the first hole transport layer to form a second hole transport layer with a thickness of 890 Å.

Then, on the second hole transport layer, the compound B6:RH-N:RD were co-evaporated at a ratio of 49%:49%:2% to form a red light-emitting layer (EML) with a thickness of 400 Å.

On the light-emitting layer, the compound ET-2 and LiQ were co-evaporated at an evaporation rate ratio of 1:1 to form a 350 Å thick electron transport layer (ETL), and Yb was evaporated on the electron transport layer to form an electron injection layer (EIL) with a thickness of 10 Å. Then, magnesium (Mg) and silver (Ag) were mixed at an evaporation rate of 1:9 and vacuum evaporated on the electron injection layer to form a cathode with a thickness of 130 Å.

In addition, CP with a thickness of 800 Å was vacuum evaporated on the above cathode to complete the fabrication of the red organic electroluminescent device.

### Examples 35 to 60

Organic electroluminescent devices were fabricated by the same method as used in Example 34, except that Compound B6 in Example 34 was replaced with Compound Y in the following Table 12 when an light-emitting layer was formed.

### Comparative Examples 4 to 6

Organic electroluminescent devices were fabricated by the same method as used in Example 34, except that Compound B6 in Example 34 was replaced with Compound D, Compound E, or Compound F when an light-emitting layer was formed.

Structures of the materials used in the examples and comparative examples are shown in below.

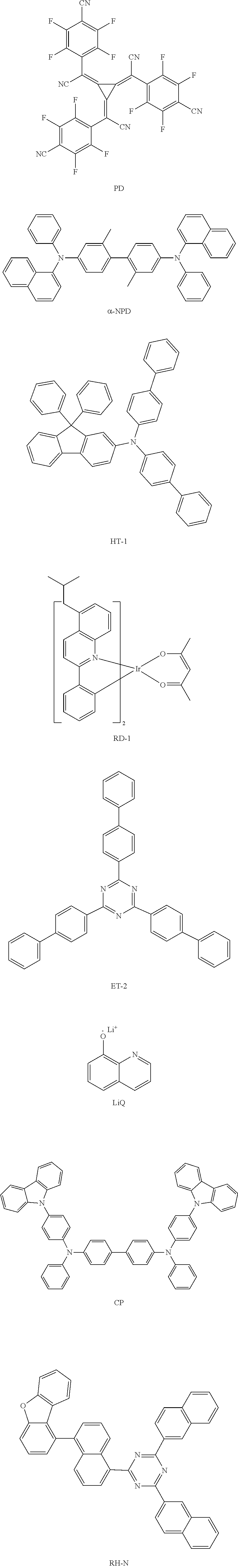

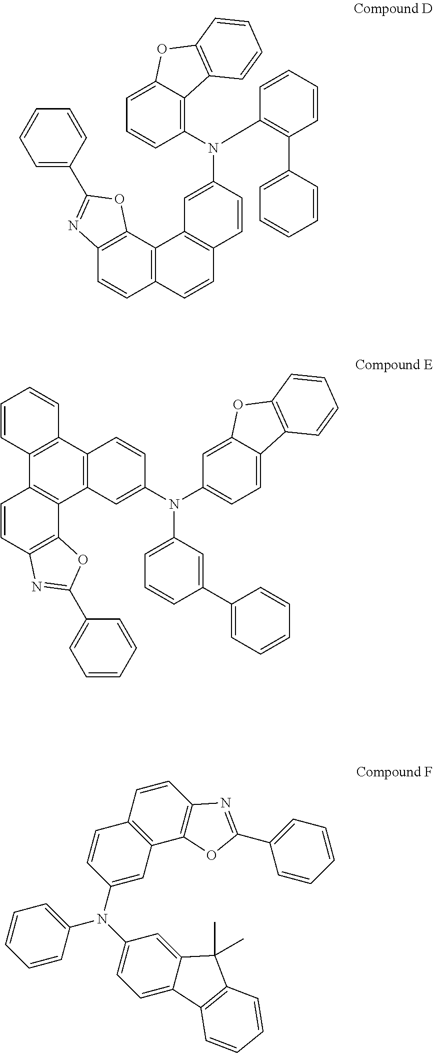 [SMILES: c1ccc(-c2nc3ccc4ccc5ccc(N(c6ccccc6-c6ccccc6)c6cccc7oc8ccccc8c67)cc5c4c3o2)cc1 ; c1ccc(-c2cccc(N(c3ccc4c(c3)oc3ccccc34)c3ccc4c5ccccc5c5ccc6nc(-c7ccccc7)oc6c5c4c3)c2)cc1 ; CC1(C)c2ccccc2-c2ccc(N(c3ccccc3)c3ccc4ccc5nc(-c6ccccc6)oc5c4c3)cc21]

The red organic electroluminescent devices fabricated in the Examples 34 to 60 and Comparative Examples 4 to 6 were tested for their performance. Specifically, the IVL characteristics of the devices were tested under the condition of 10 mA/cm², and the T95 lifetime of the devices were tested under the condition of 20 mA/cm². Test results are shown in Table 12.

TABLE 12

| | Light-emitting | Operating | | | | |
|---|---|---|---|---|---|---|
| Example | Compound Y: | voltage | | | | T95(hrs) |
| number | RH-N:RD | Volt (V) | Cd/A | CIEx | CIEy | @20 mA/cm² |
| Example 34 | Compound B6 | 3.47 | 61.6 | 0.680 | 0.320 | 507 |
| Example 35 | Compound B18 | 3.47 | 61.7 | 0.680 | 0.320 | 491 |
| Example 36 | Compound B26 | 3.50 | 62.2 | 0.680 | 0.320 | 509 |
| Example 37 | Compound B32 | 3.45 | 62.7 | 0.680 | 0.320 | 503 |
| Example 38 | Compound B34 | 3.45 | 61.5 | 0.680 | 0.320 | 492 |
| Example 39 | Compound B42 | 3.49 | 63.5 | 0.680 | 0.320 | 503 |
| Example 40 | Compound B49 | 3.50 | 61.7 | 0.680 | 0.320 | 487 |
| Example 41 | Compound B51 | 3.48 | 62.1 | 0.680 | 0.320 | 496 |
| Example 42 | Compound B60 | 3.48 | 63.4 | 0.680 | 0.320 | 508 |
| Example 43 | Compound B61 | 3.50 | 63.0 | 0.680 | 0.320 | 488 |
| Example 44 | Compound B72 | 3.49 | 63.3 | 0.680 | 0.320 | 505 |
| Example 45 | Compound B73 | 3.50 | 61.8 | 0.680 | 0.320 | 509 |
| Example 46 | Compound B82 | 3.45 | 62.1 | 0.680 | 0.320 | 494 |
| Example 47 | Compound B94 | 3.49 | 61.5 | 0.680 | 0.320 | 509 |
| Example 48 | Compound B100 | 3.50 | 62.7 | 0.680 | 0.320 | 524 |
| Example 49 | Compound B103 | 3.48 | 62.6 | 0.680 | 0.320 | 502 |
| Example 50 | Compound B117 | 3.50 | 61.8 | 0.680 | 0.320 | 501 |
| Example 51 | Compound B124 | 3.49 | 61.9 | 0.680 | 0.320 | 506 |
| Example 52 | Compound B132 | 3.49 | 63.2 | 0.680 | 0.320 | 491 |
| Example 53 | Compound B150 | 3.45 | 61.9 | 0.680 | 0.320 | 490 |
| Example 54 | Compound B170 | 3.46 | 53.5 | 0.680 | 0.320 | 512 |
| Example 55 | Compound B171 | 3.45 | 53.3 | 0.680 | 0.320 | 489 |
| Example 56 | Compound B188 | 3.49 | 59.2 | 0.680 | 0.320 | 490 |
| Example 57 | Compound B210 | 3.48 | 60.4 | 0.680 | 0.320 | 470 |
| Example 58 | Compound B211 | 3.48 | 62.3 | 0.680 | 0.320 | 468 |
| Example 59 | Compound B215 | 3.50 | 53.6 | 0.680 | 0.320 | 473 |
| Example 60 | Compound B230 | 3.44 | 62.5 | 0.680 | 0.320 | 493 |
| Comparative | Compound D | 3.53 | 46.2 | 0.680 | 0.320 | 426 |
| Example 4 | | | | | | |
| Comparative | Compound E | 3.51 | 47.1 | 0.680 | 0.320 | 415 |
| Example 5 | | | | | | |
| Comparative | Compound F | 3.54 | 44.1 | 0.680 | 0.320 | 402 |
| Example 6 | | | | | | |

According to Table 12 above, when the compound of the present disclosure is used as a hole-transporting host material in the host material of the light-emitting layer of the red organic electroluminescent device, the luminous efficiency of the device is improved by at least 13.1% and the lifetime of the device is improved by at least 9.9%.

The above combined with the attached figures describes in detail the preferred implementation method of the invention. However, the invention is not limited to the specific details in the above implementation method. Within the scope of the technical conception of the invention, a variety of simple variants of the technical scheme of the invention can be carried out. These simple variants belong to the scope of protection of the invention.

1. A nitrogen-containing compound, having a structure shown in Formula 1: 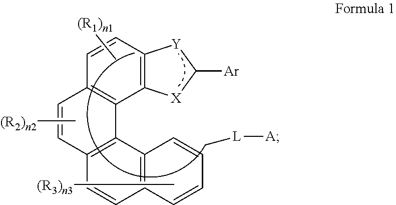
wherein, group A is selected from Formula (A-1) or Formula (A-2); 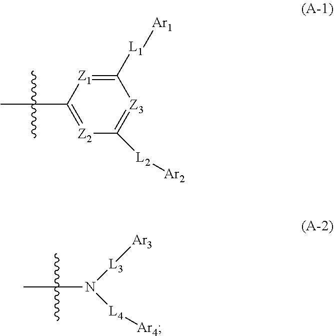
one of X and Y is N═, and the other is O or S;
Z₁ and Z₃ are N, and Z₂ is selected from C(H) or N; or Z₁ and Z₂ are N, and Z₃ is selected from C(H) or N;
L, L₁, L₂, L₃, and L₄ are identical or different, and are each independently selected from a single bond, a substituted or unsubstituted arylene having 6 to 30 carbon atoms, and a substituted or unsubstituted heteroarylene having 3 to 30 carbon atoms;
Ar, Ar₁, Ar₂, Ar₃, and Ar₄ are identical or different, and are each independently selected from a substituted or unsubstituted aryl having 6 to 40 carbon atoms, and a substituted or unsubstituted heteroaryl having 3 to 40 carbon atoms;
Substituent(s) in L, L₁, L₂, L₃, L₄, Ar, Ar₁, Ar₂, Ar₃, and Ar₄ are identical or different, and are each independently selected from deuterium, cyano, a halogen, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, a deuterated aryl having 6 to 20 carbon atoms, a heteroaryl having 3 to 20 carbon atoms, a cycloalkyl having 3 to 10 carbon atoms, an alkoxy having 1 to 10 carbon atoms, an alkylthio having 1 to 10 carbon atoms, an aryloxy having 6 to 20 carbon atoms, and an arylthio having 6 to 20 carbon atoms; optionally, any two adjacent substituents form a ring;
R₁, R₂, and R₃ are identical or different, and are each independently selected from hydrogen, deuterium, cyano, a halogen, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, a heteroaryl having 3 to 20 carbon atoms, and a cycloalkyl having 3 to 10 carbon atoms; n₁ and n₂ are each independently selected from 0, 1, and 2, and n₃ is selected from 0, 1, 2, 3, 4, 5, and 6.

2. The nitrogen-containing compound according to claim 1, wherein the structure of the nitrogen-containing is selected from Formulae (1-1) to (1-16):

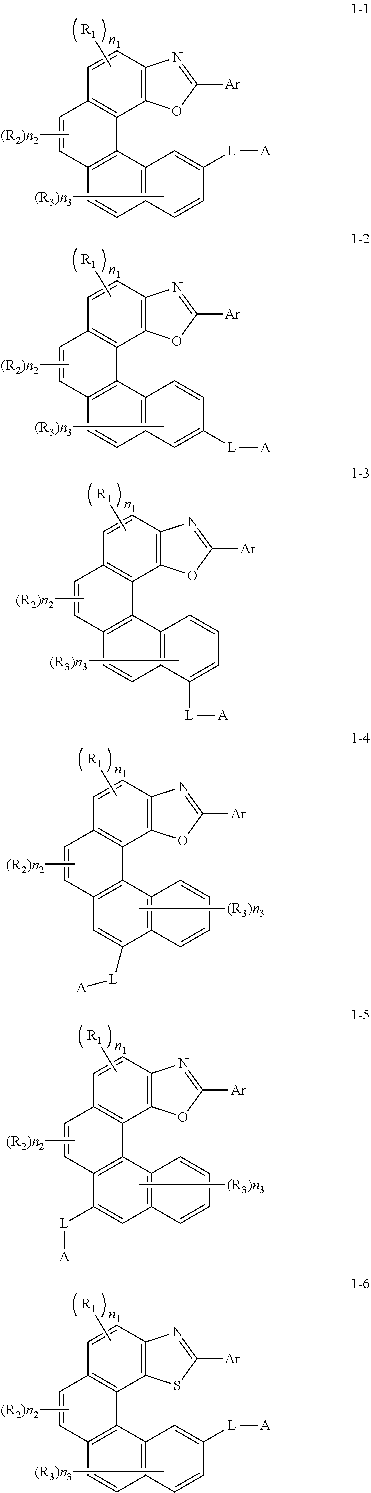

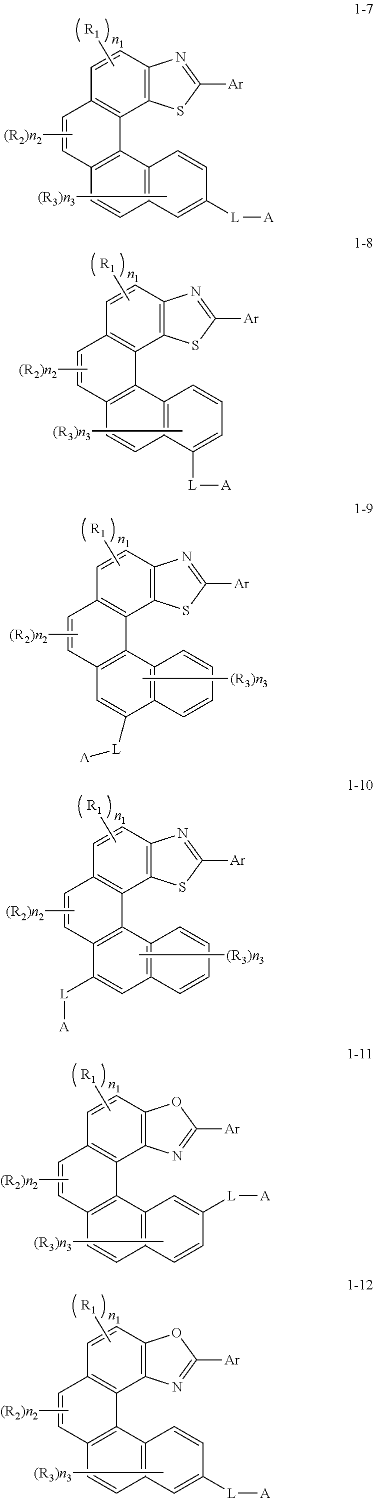

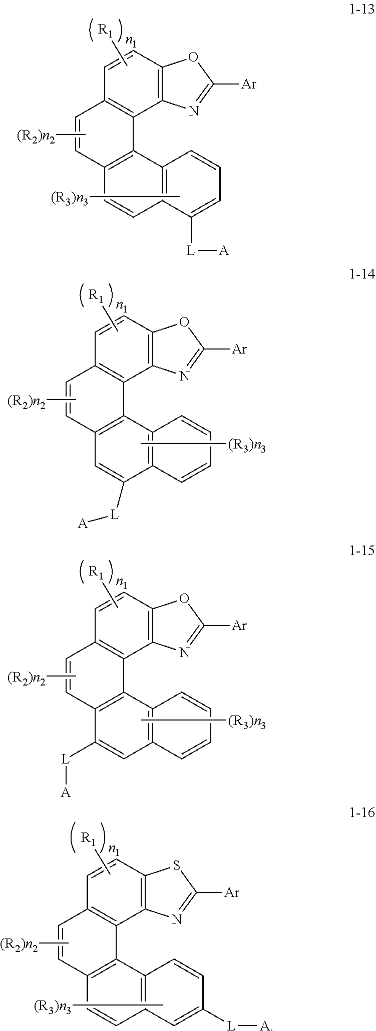

3. The nitrogen-containing compound according to claim 1, wherein Ar, Ar₁, Ar₂, Ar₃, and Ar₄ are identical or different, and are each independently selected from a substituted or unsubstituted aryl having 6 to 25 carbon atoms, and a substituted or unsubstituted heteroaryl having 5 to 24 carbon atoms;
optionally, substituent(s) in Ar, Ar₁, Ar₂, Ar₃, or Ar₄ are each independently selected from deuterium, a halogen, cyano, a haloalkyl having 1 to 4 carbon atoms, a deuterated alkyl having 1 to 4 carbon atoms, an alkyl having 1 to 4 carbon atoms, a cycloalkyl having 5 to 10 carbon atoms, an aryl having 6 to 15 carbon atoms, a heteroaryl having 5 to 12 carbon atoms, a trialkylsilyl having 3 to 8 carbon atoms, and a deuterated aryl having 6 to 15 carbon atoms, optionally, any two adjacent substituents form a benzene ring or a fluorene ring.

4. The nitrogen-containing compound according to claim 1, wherein Ar, Ar₁, Ar₂, Ar₃, and Ar₄ are identical or different, and are each independently selected from a substituted or unsubstituted group W; wherein the unsubstituted group W is selected from the group consisting of the following groups: 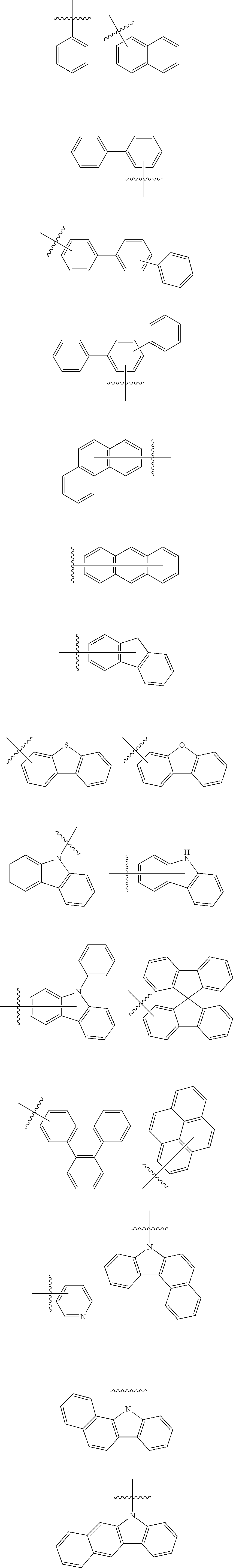 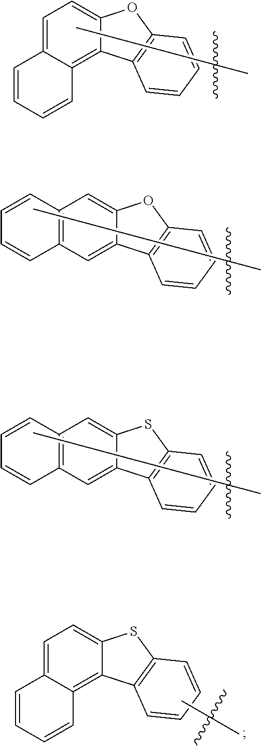
the substituted group W has one or more substituent(s), and each substituent is independently selected from deuterium, fluorine, cyano, trideuteromethyl, trimethylsilyl, trifluoromethyl, cyclopentyl, cyclohexyl, adamantyl, methyl, ethyl, isopropyl, tert-butyl, phenyl, naphthyl, pyridyl, dibenzofuranyl, dibenzothienyl and carbazolyl, and when the number of the substituents is greater than 1, the substituents are identical or different.

5. The nitrogen-containing compound according to claim 1, wherein Ar is selected from a substituted or unsubstituted phenyl, a substituted or unsubstituted biphenyl, a substituted or unsubstituted terphenyl, a substituted or unsubstituted naphthyl, a substituted or unsubstituted phenanthryl, a substituted or unsubstituted fluorenyl, a substituted or unsubstituted triphenylene, a substituted or unsubstituted dibenzothienyl, a substituted or unsubstituted dibenzofuranyl, and a substituted or unsubstituted carbazolyl;
optionally, substituent(s) in Ar are independently selected from deuterium, fluorine, cyano, trideuteromethyl, trimethylsilyl, trifluoromethyl, methyl, ethyl, isopropyl, tert-butyl, phenyl, naphthyl, and deuterophenyl.

6. The nitrogen-containing compound according to claim 1, wherein, Ar₁, Ar₂, Ar₃ and Ar₄ are identical or different, and are independently selected from the group consisting of the following groups:

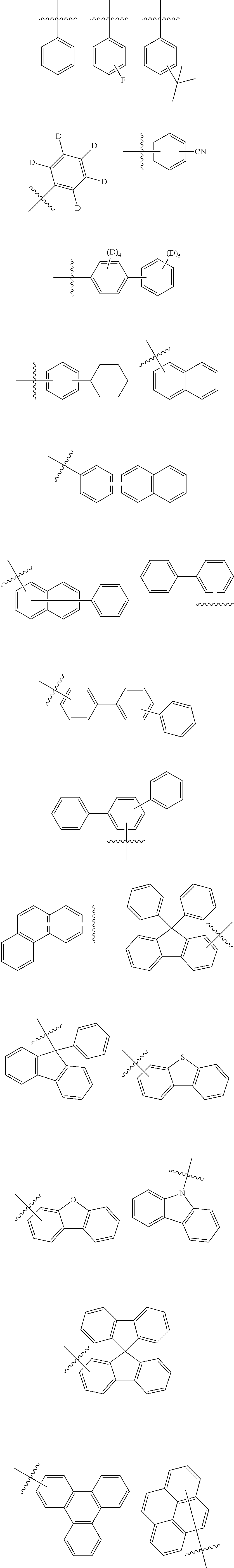

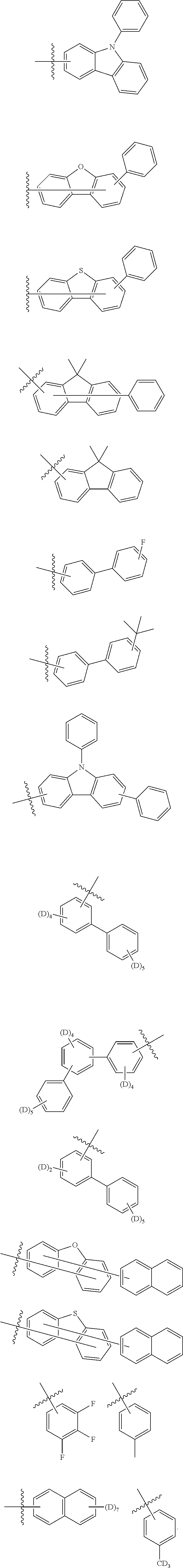

7. The nitrogen-containing compound according to claim 1, wherein L, L₁, L₂, L₃, and L₄ are identical or different, and are each independently selected from a single bond, a substituted or unsubstituted phenylene, a substituted or unsubstituted naphthylene, a substituted or unsubstituted biphenylene, a substituted or unsubstituted dibenzothienylene, a substituted or unsubstituted dibenzofuranylene, a substituted or unsubstituted fluorenylene, a substituted or unsubstituted phenanthrylene, and a substituted or unsubstituted carbazolylene;
optionally, substituents in L, L₁, L₂, L₃, and L₄ are identical or different, and are each independently selected from deuterium, fluorine, cyano, methyl, ethyl, isopropyl, tert-butyl, trifluoromethyl, trideuteromethyl, trimethylsilyl, and phenyl.

8. The nitrogen-containing compound according to claim 1, wherein L is selected from a single bond or the group consisting of the following groups: 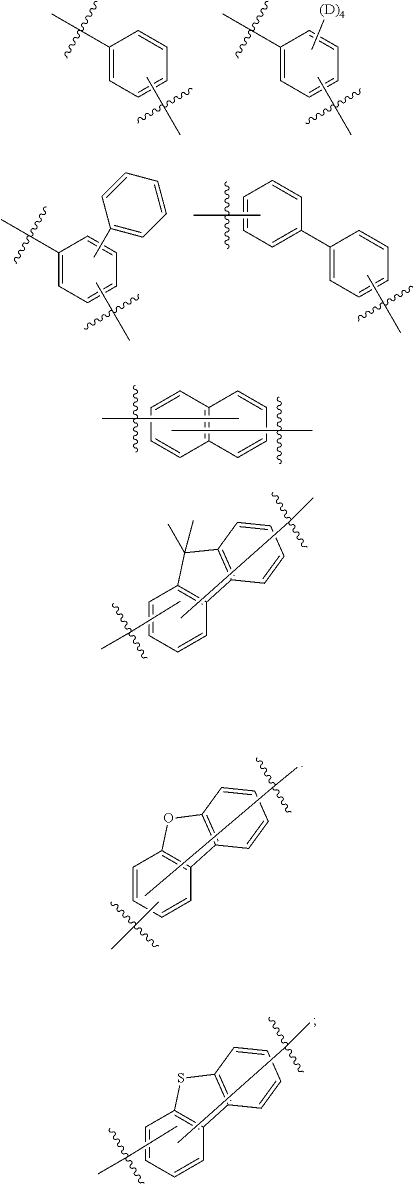
optionally, L₁, L₂, L₃, and L₄ are each independently selected from a single bond or the group consisting of the following groups:

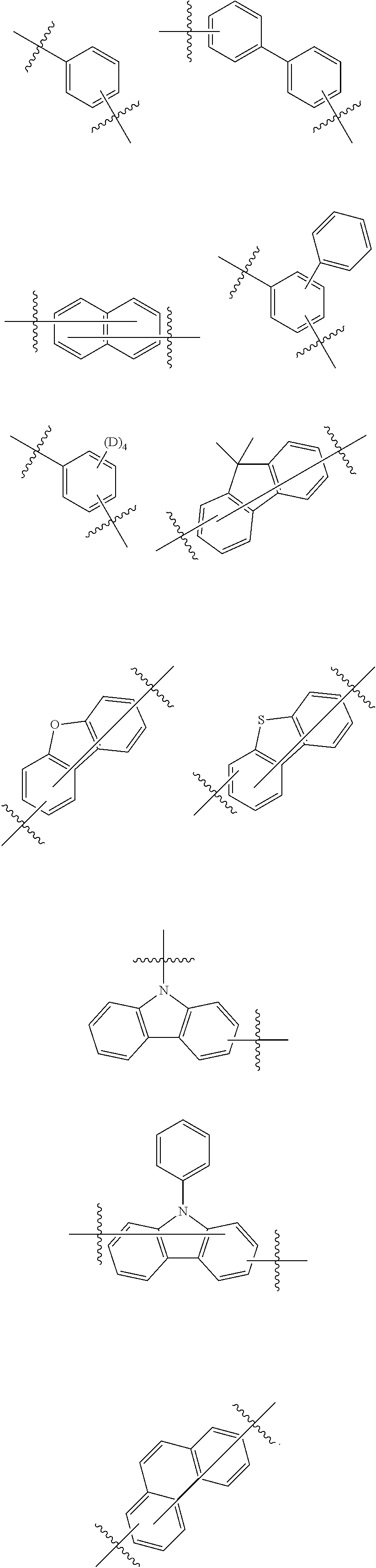

9. The nitrogen-containing compound according to claim 1, wherein L is selected from a single bond or the group consisting of the following groups: 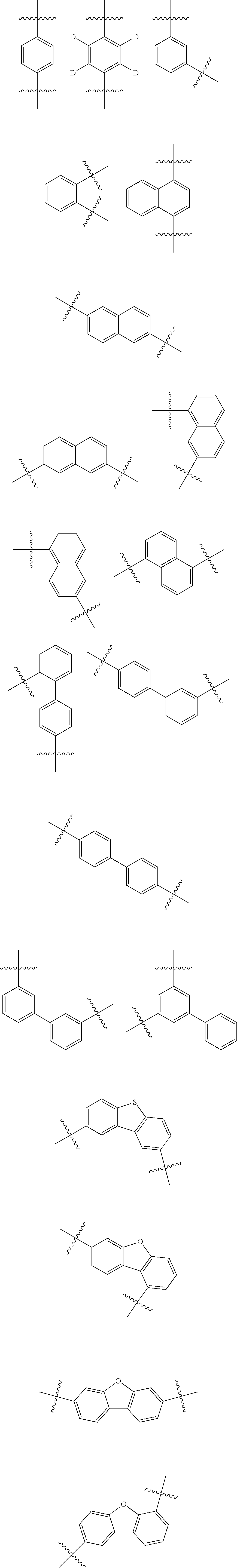 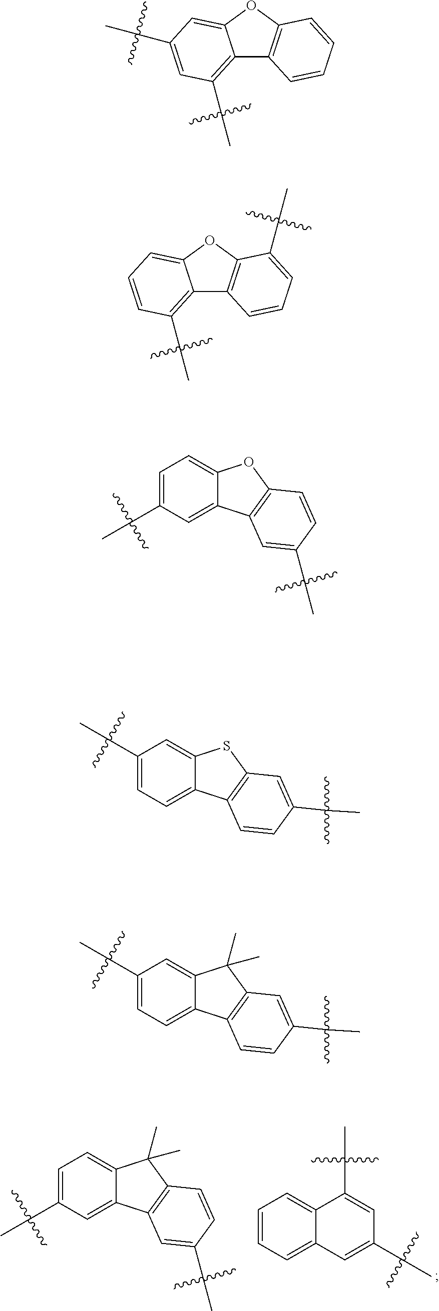
optionally, L₁, L₂, L₃, and L₄ are identical or different, and are each independently selected from a single bond or the group consisting of the following groups:

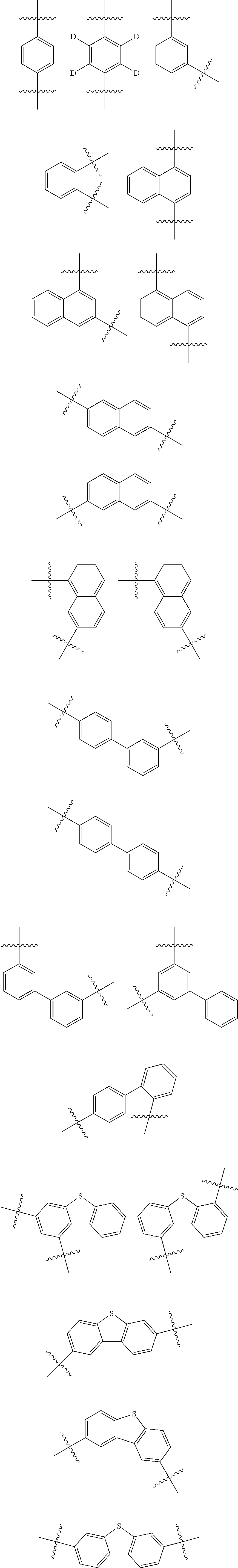

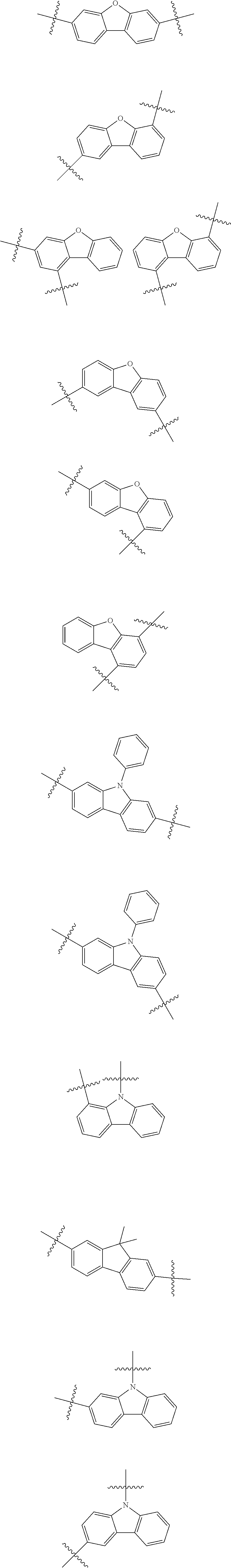

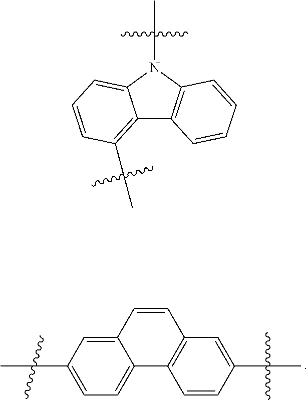 [SMILES: *c1cccc2c1c1ccccc1n2* ; *c1ccc2c(ccc3cc(*)ccc32)c1]

10. The nitrogen-containing compound according to claim 1, wherein 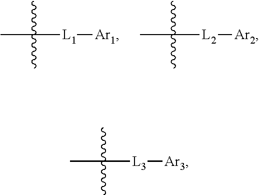
and 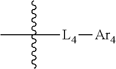
are each independently selected from the following groups:

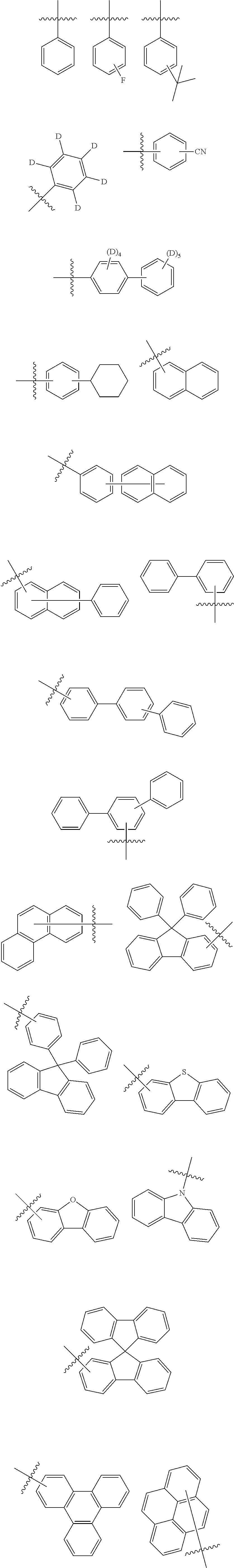

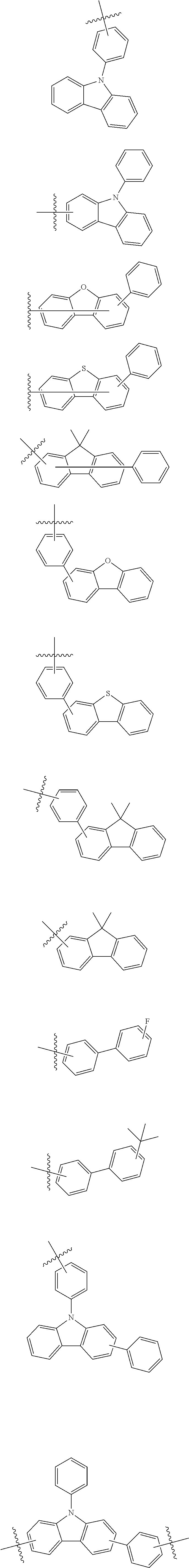

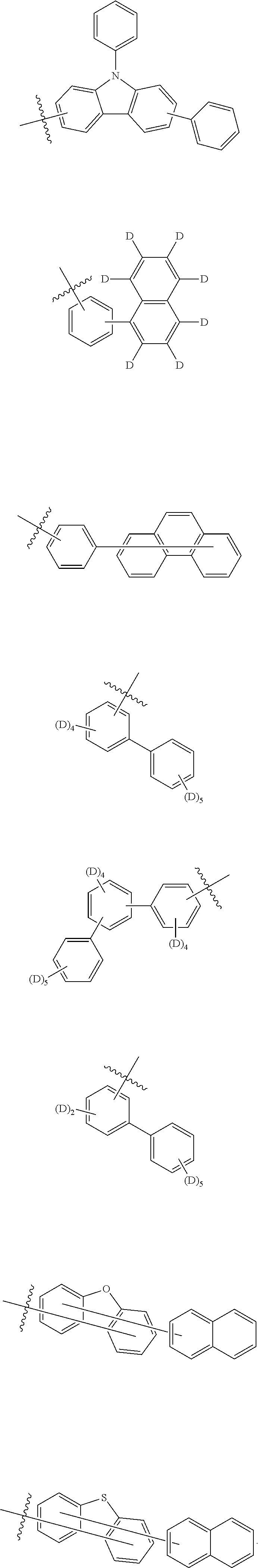

11. The nitrogen-containing compound according to claim 1, wherein Ar is selected from the following groups:

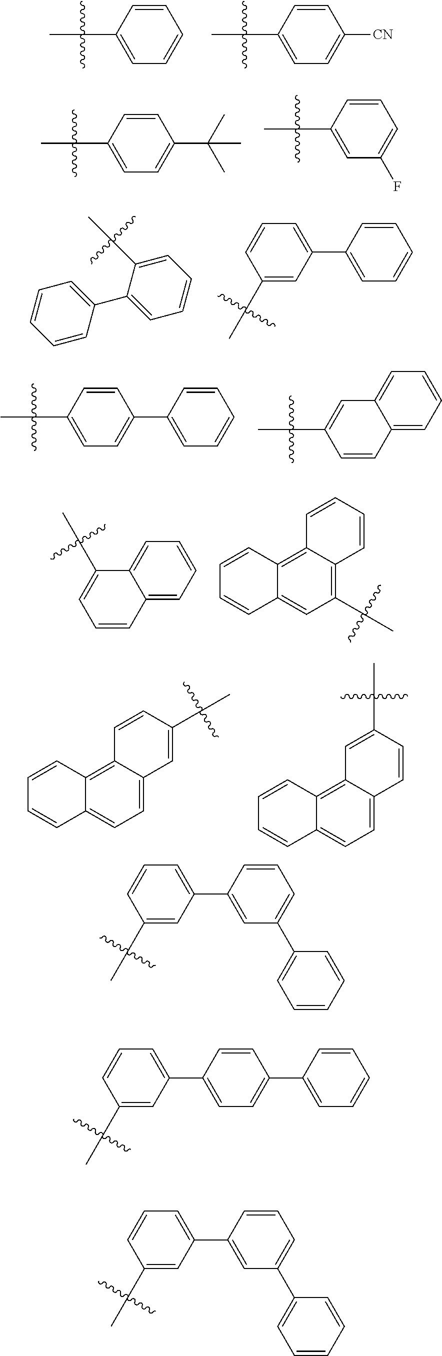

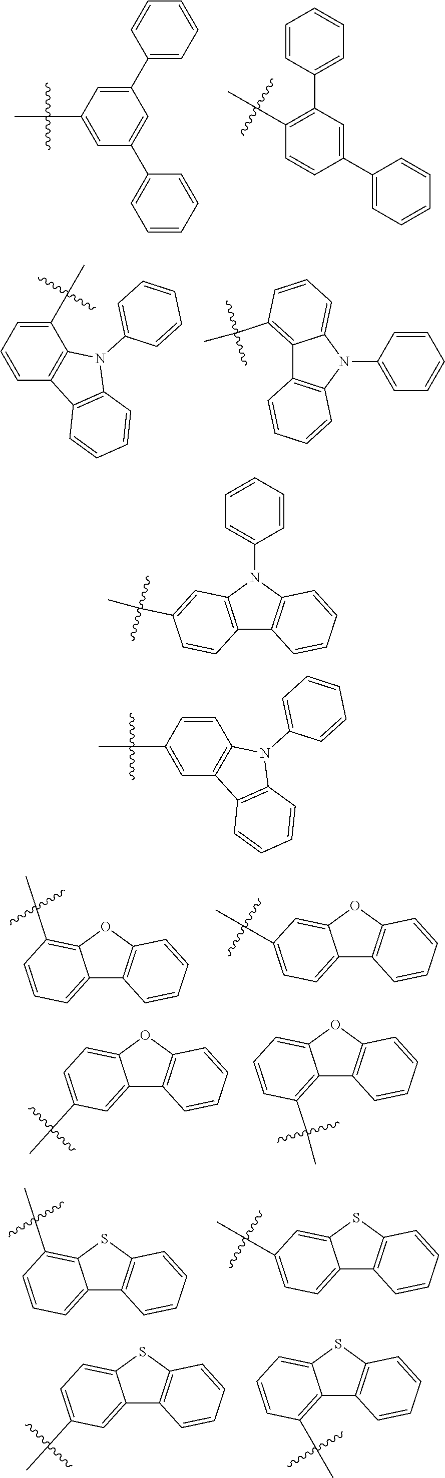

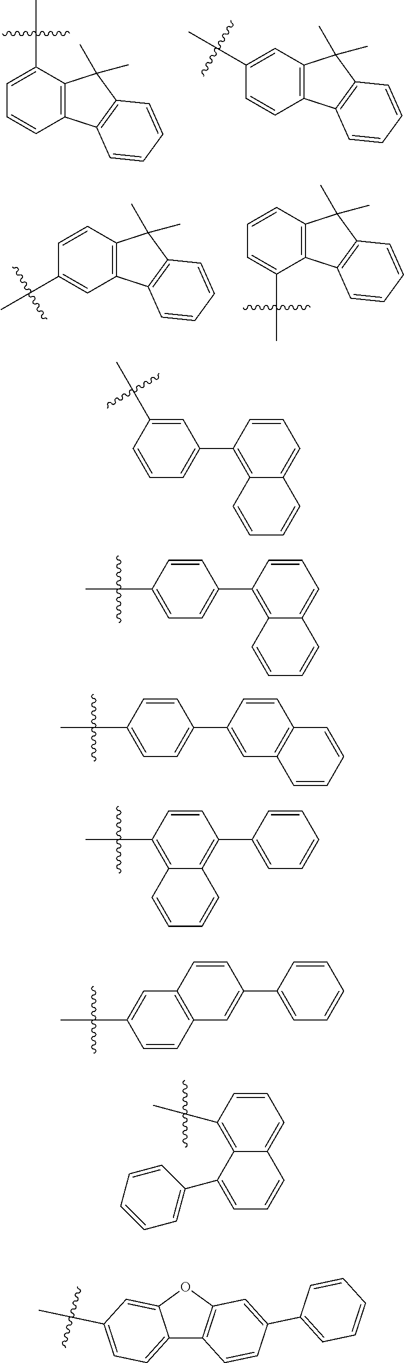

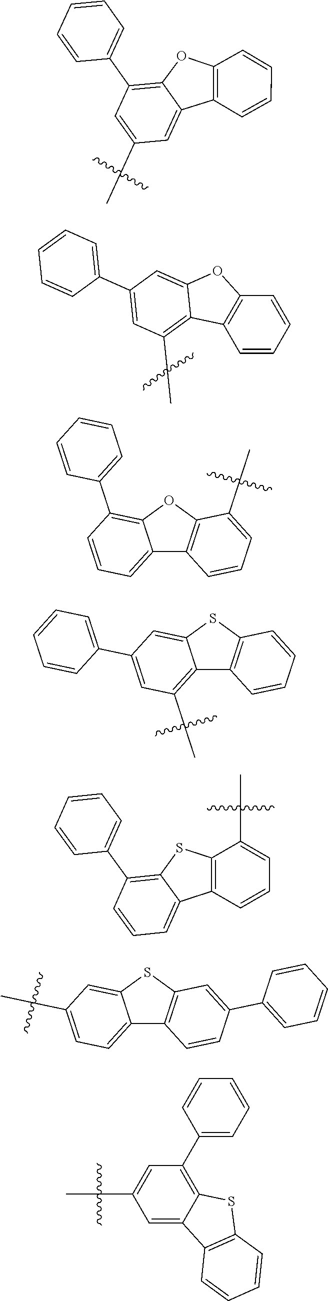

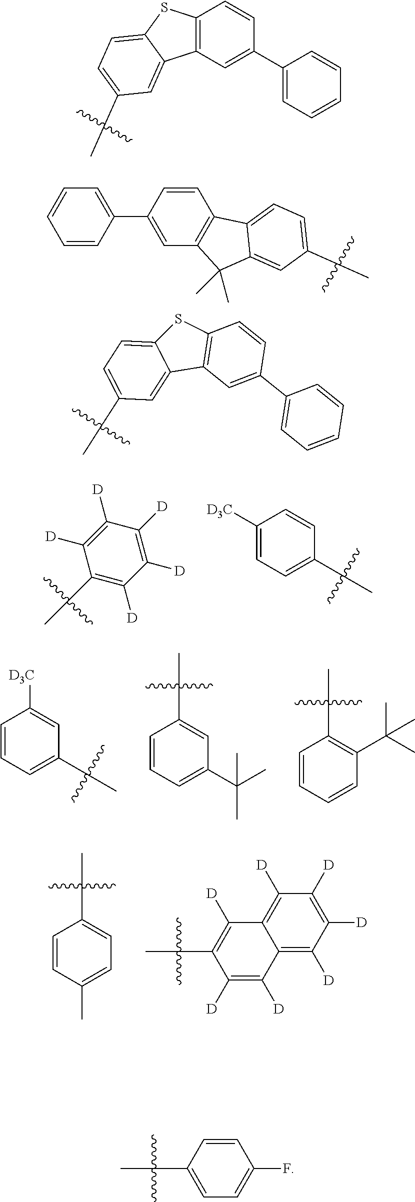

12. The nitrogen-containing compound according to claim 1, wherein R₁, R₂, and R₃ are identical or different, and are each independently selected from hydrogen, deuterium, cyano, fluorine, trideuteromethyl, trimethylsily, trifluoromethyl, methyl, ethyl, isopropyl, tert-butyl, phenyl, naphthyl, pyridyl, dibenzofuranyl, dibenzothienyl, and carbazolyl.

13. The nitrogen-containing compound according to claim 1, wherein group A is selected from the following groups:

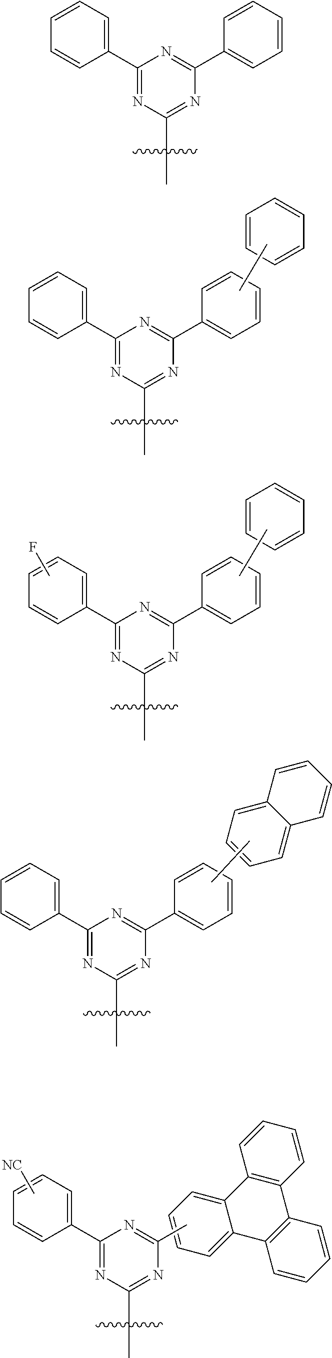

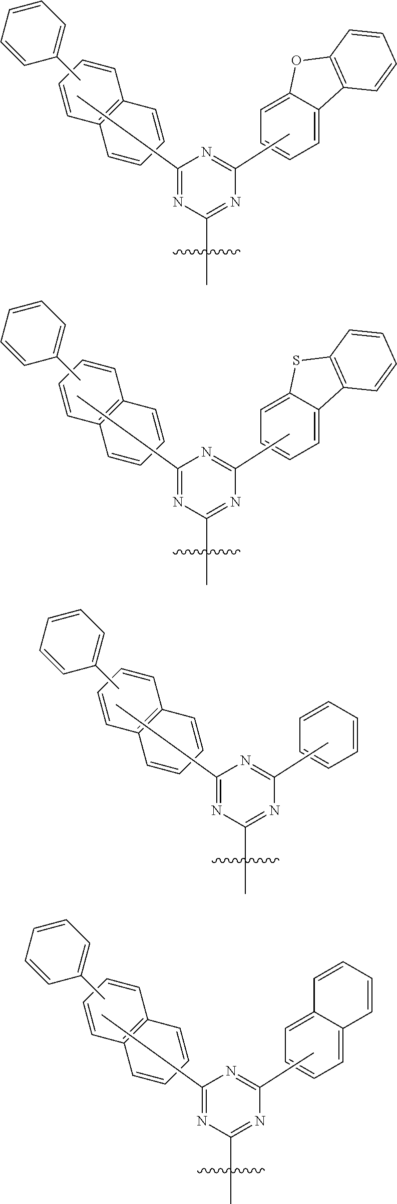

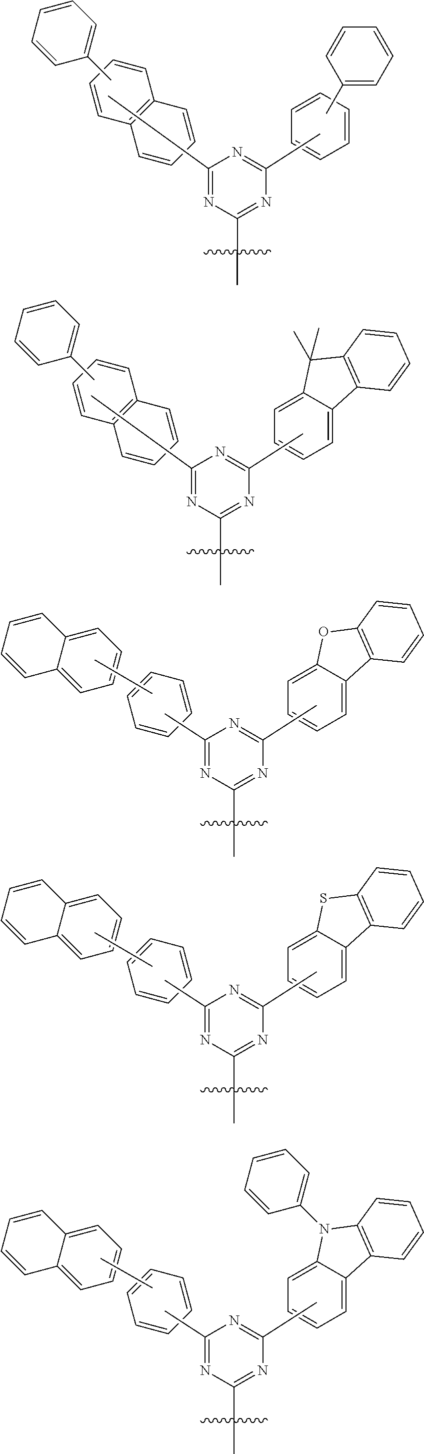

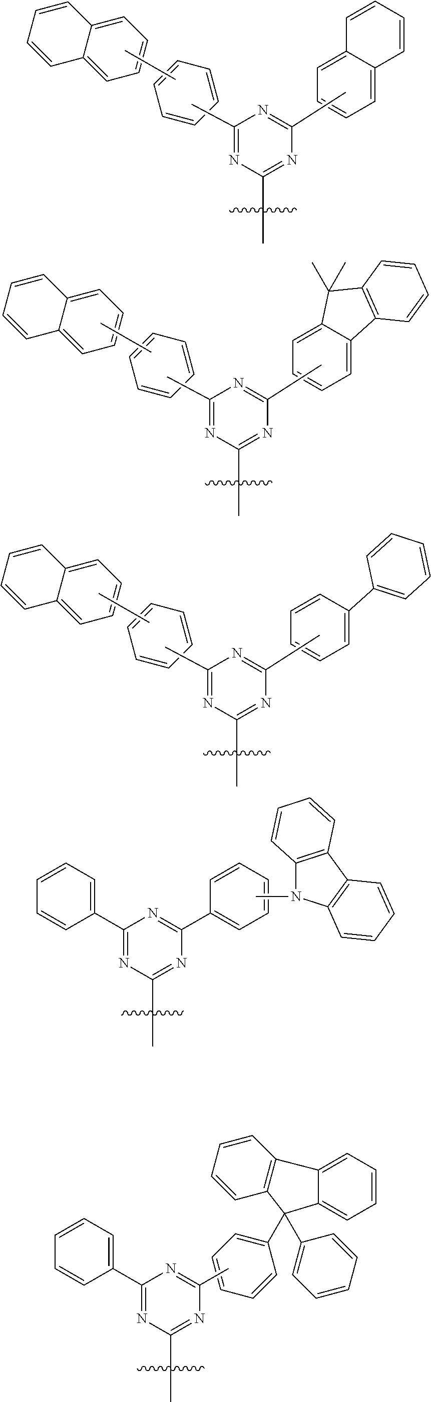

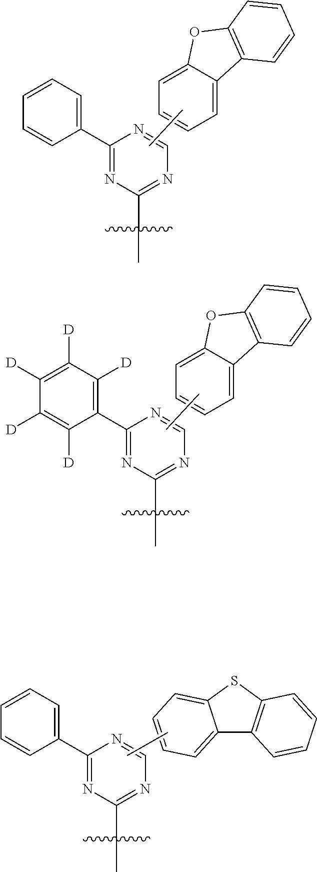

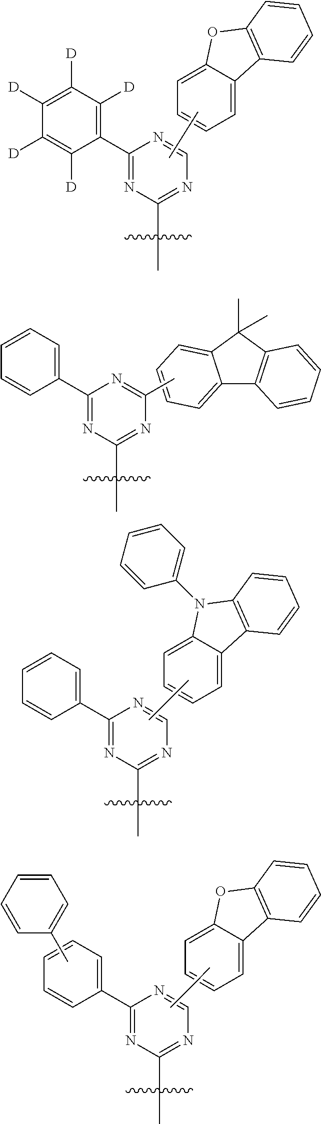

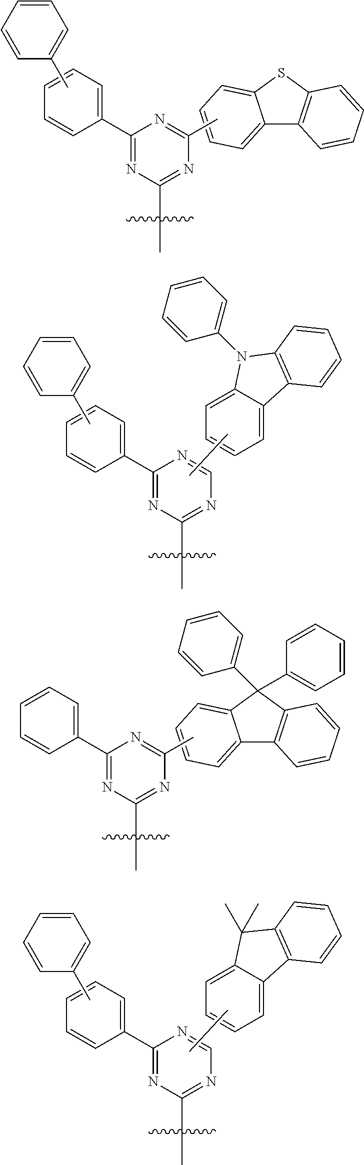

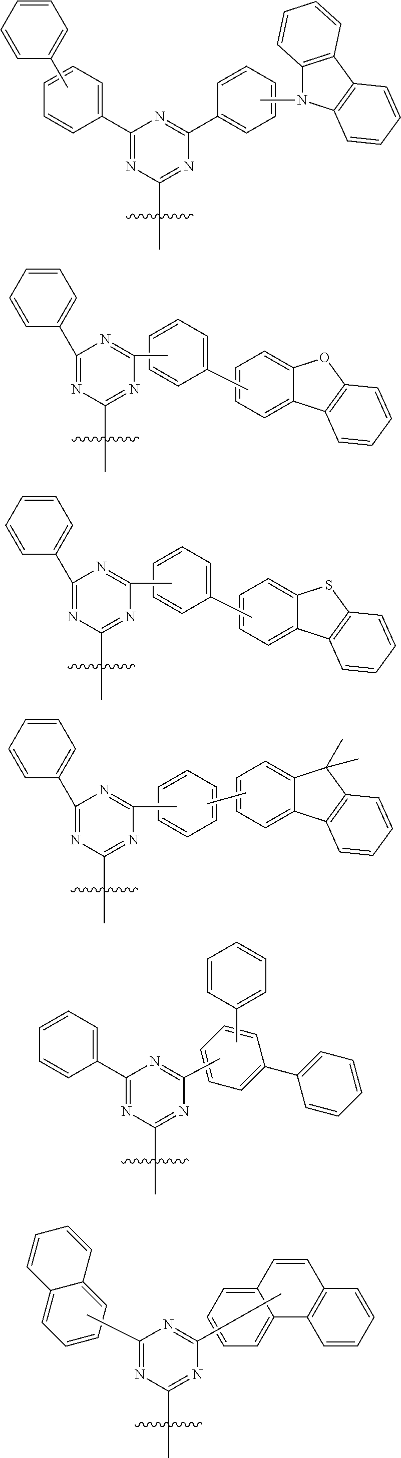

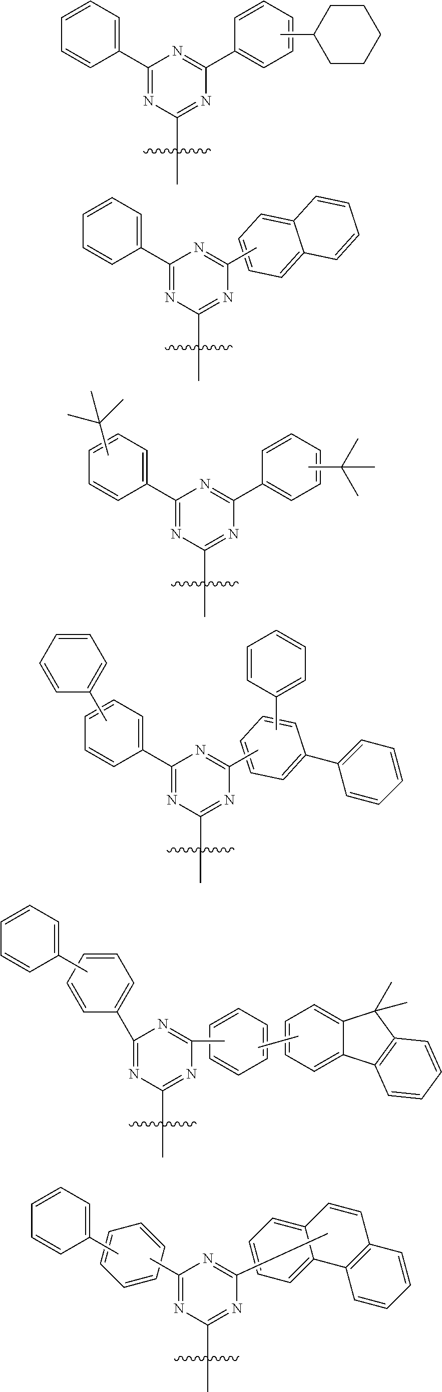

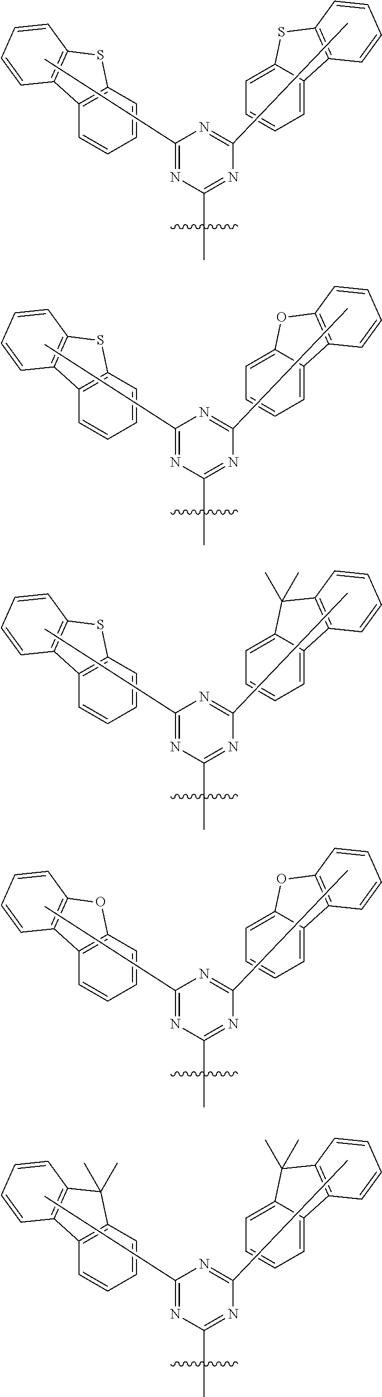

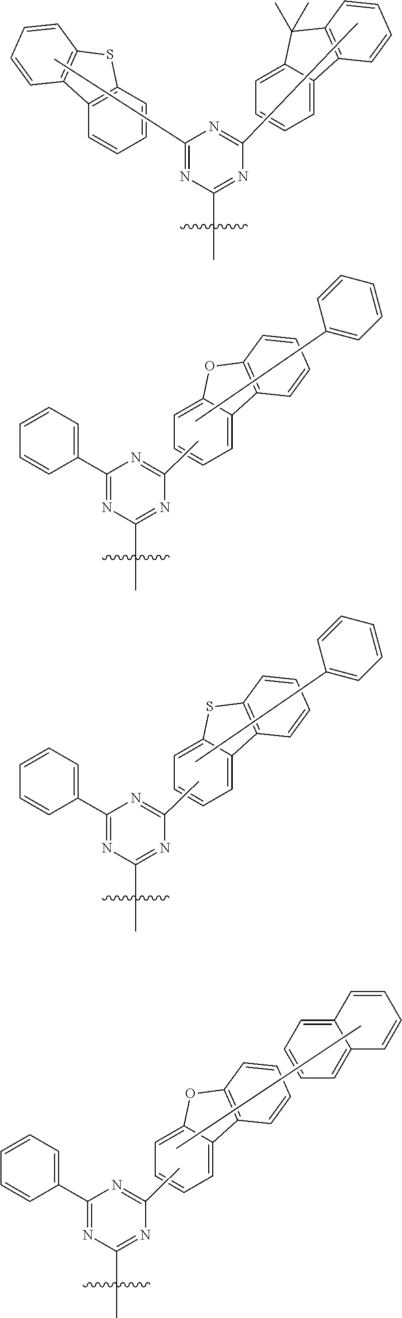

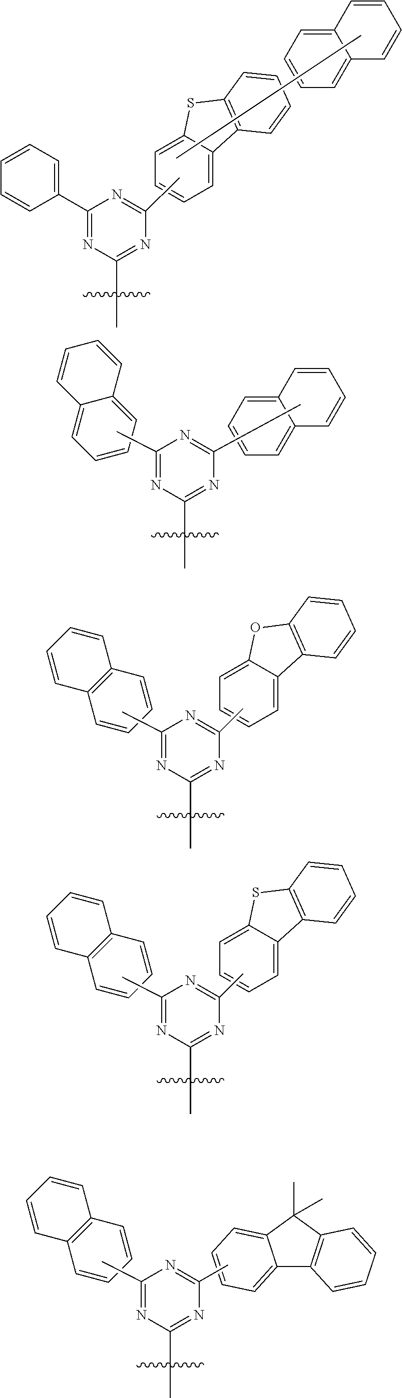

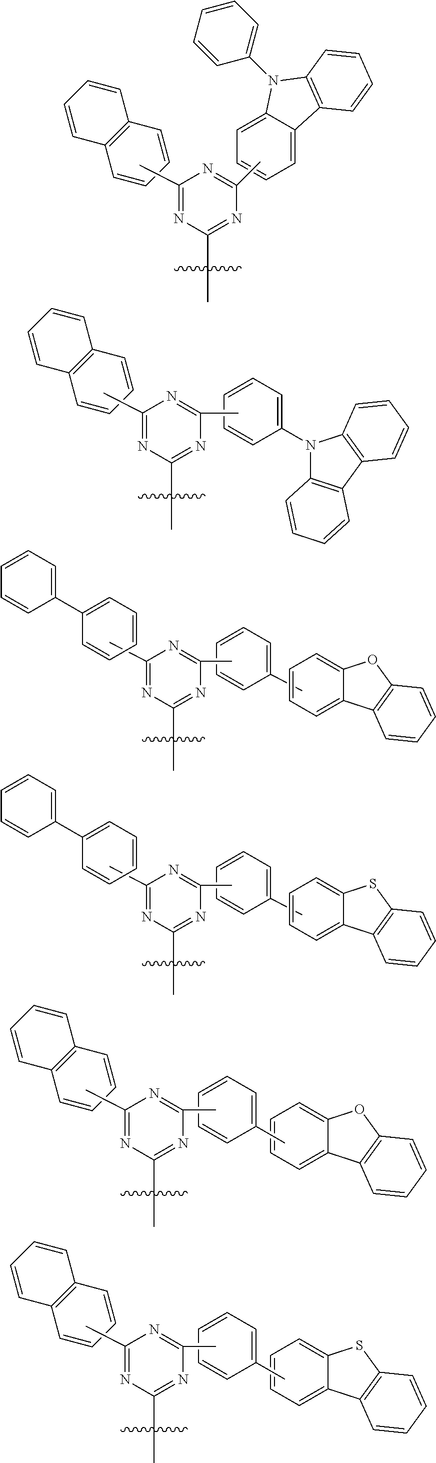

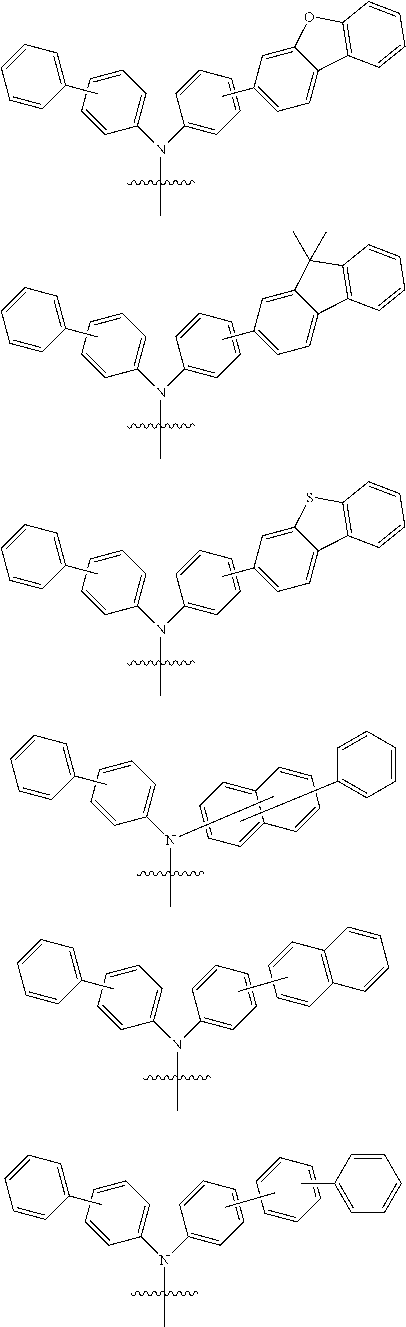

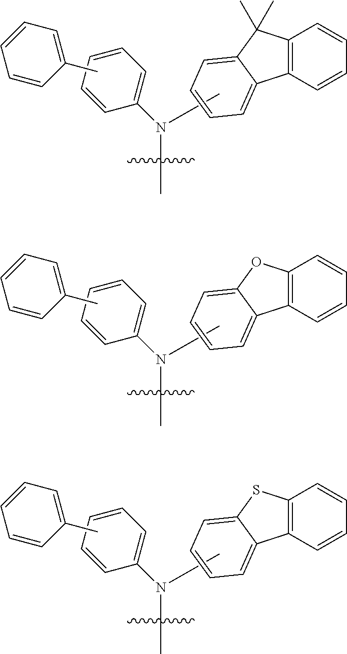

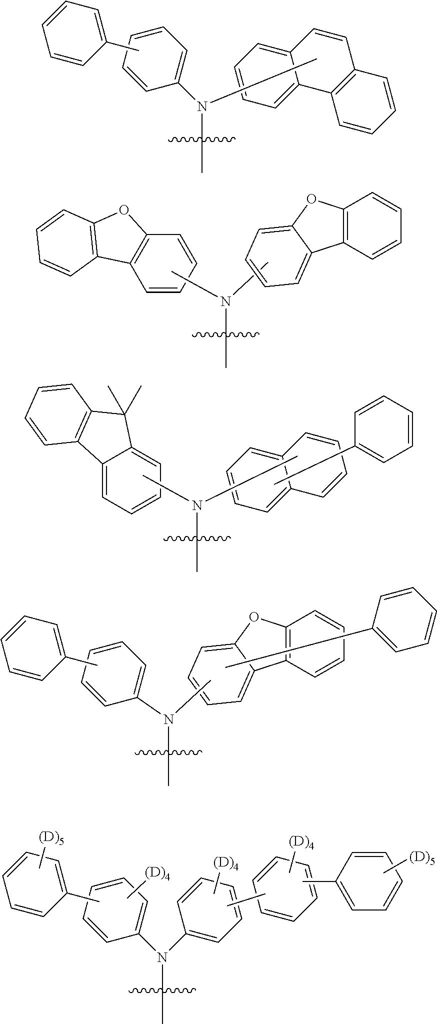

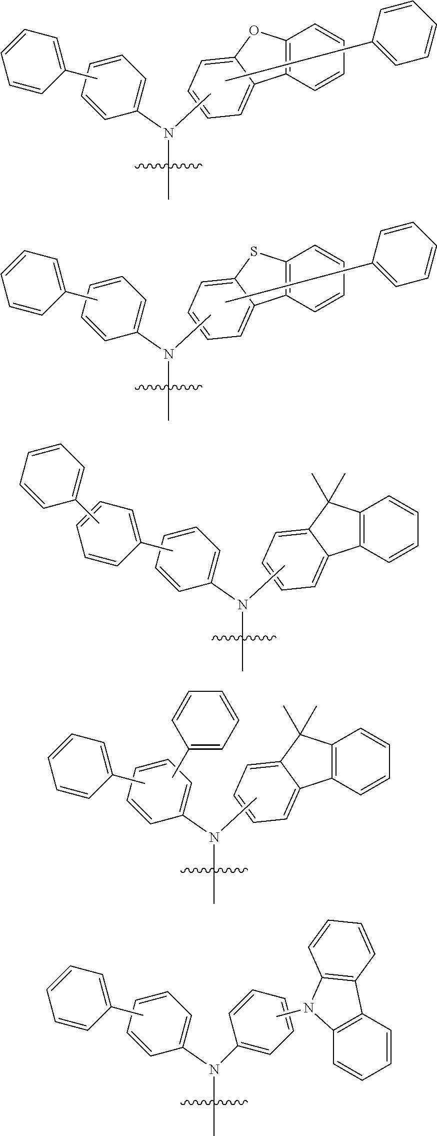

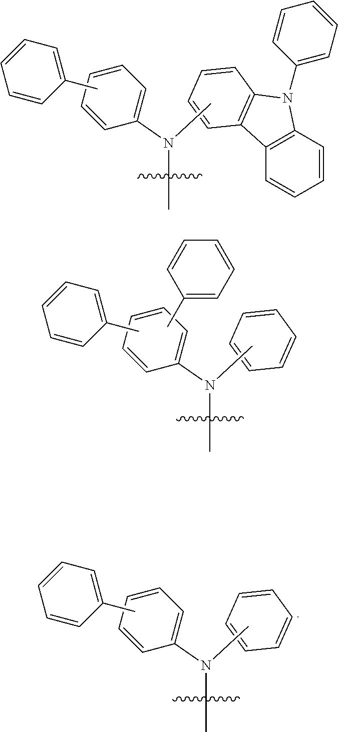

14. The nitrogen-containing compound according to claim 1, wherein the nitrogen-containing compound is selected from the group consisting of the following groups:

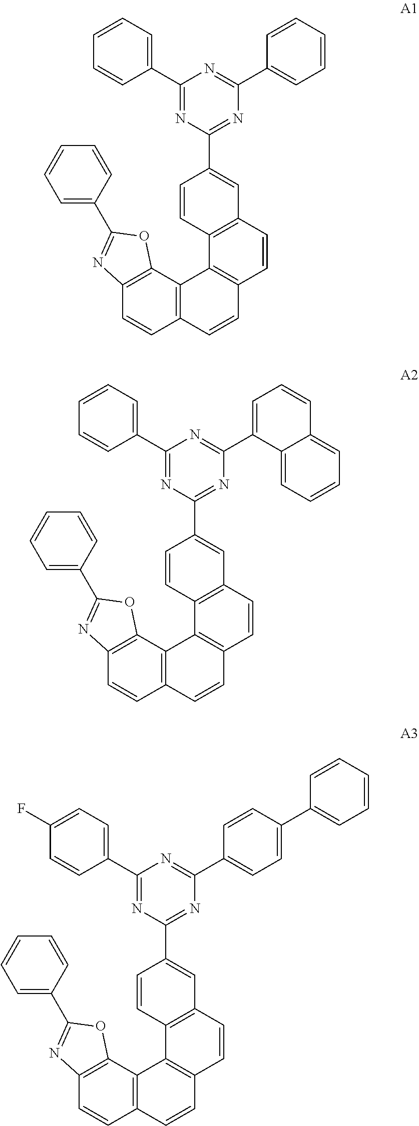 [SMILES: c1ccc(-c2nc(-c3ccccc3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3cccc4ccccc34)n2)cc1 ; Fc1ccc(-c2nc(-c3ccc(-c4ccccc4)cc3)nc(-c3ccc4c(ccc5ccc6ccc7nc(-c8ccccc8)oc7c6c54)c3)n2)cc1]

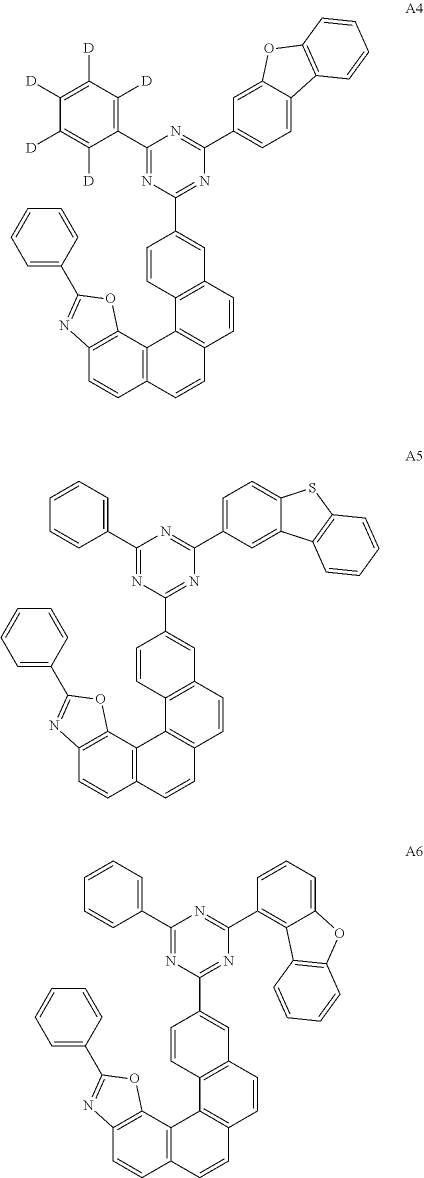 [SMILES: [2H]c1c([2H])c([2H])c(-c2nc(-c3ccc4c(ccc5ccc6ccc7nc(-c8ccccc8)oc7c6c54)c3)nc(-c3ccc4c(c3)oc3ccccc34)n2)c([2H])c1[2H] ; c1ccc(-c2nc(-c3ccc4c(ccc5ccc6ccc7nc(-c8ccccc8)oc7c6c54)c3)nc(-c3ccc4sc5ccccc5c4c3)n2)cc1 ; c1ccc(-c2nc(-c3ccc4c(ccc5ccc6ccc7nc(-c8ccccc8)oc7c6c54)c3)nc(-c3cccc4oc5ccccc5c34)n2)cc1]

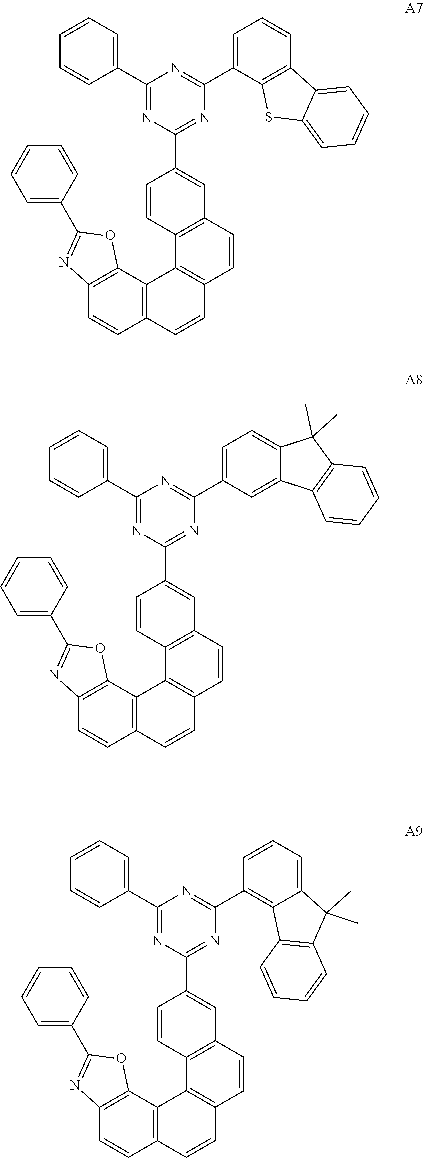 [SMILES: c1ccc(-c2nc(-c3ccc4c(ccc5ccc6ccc7nc(-c8ccccc8)oc7c6c54)c3)nc(-c3cccc4c3sc3ccccc34)n2)cc1 ; CC1(C)c2ccccc2-c2cc(-c3nc(-c4ccccc4)nc(-c4ccc5c(ccc6ccc7ccc8nc(-c9ccccc9)oc8c7c65)c4)n3)ccc21 ; CC1(C)c2ccccc2-c2c(-c3nc(-c4ccccc4)nc(-c4ccc5c(ccc6ccc7ccc8nc(-c9ccccc9)oc8c7c65)c4)n3)cccc21]

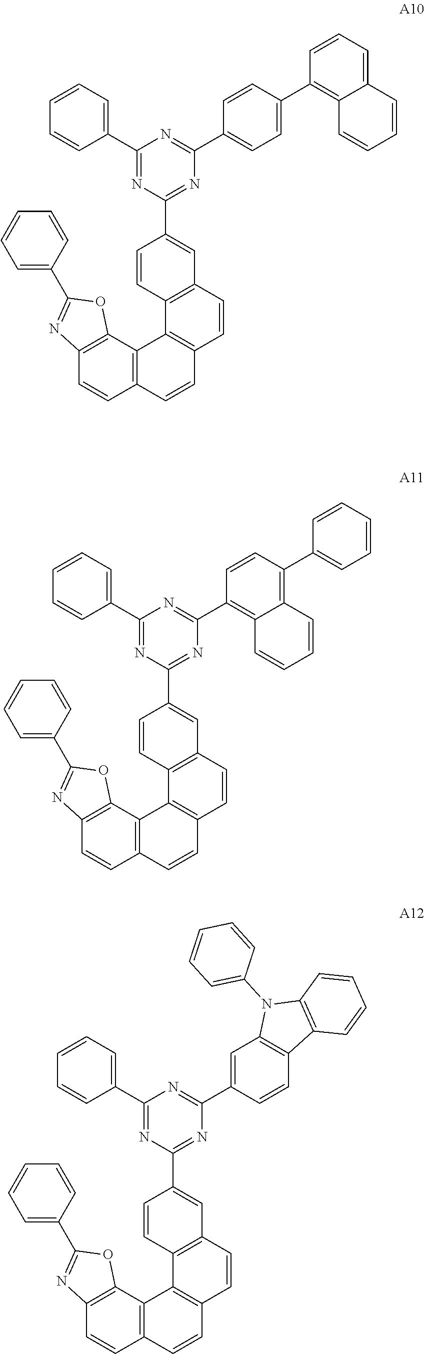 [SMILES: c1ccc(-c2nc(-c3ccc(-c4cccc5ccccc45)cc3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3ccc(-c4ccccc4)c4ccccc34)n2)cc1 ; c1ccc(-c2nc(-c3ccc4c(ccc5ccc6ccc7nc(-c8ccccc8)oc7c6c54)c3)nc(-c3ccc4c5ccccc5n(-c5ccccc5)c4c3)n2)cc1]

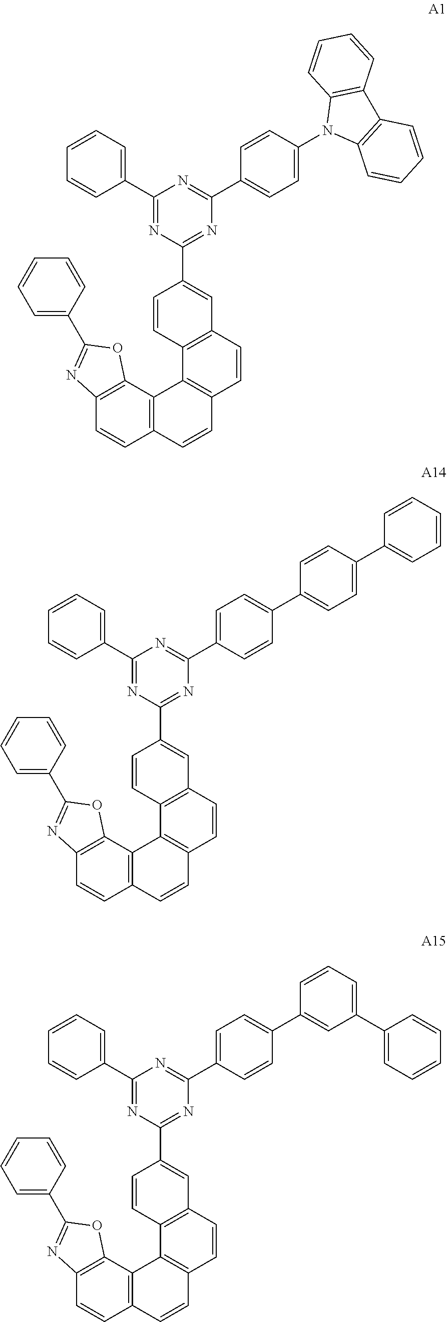 [SMILES: c1ccc(-c2nc(-c3ccc(-n4c5ccccc5c5ccccc54)cc3)nc(-c3ccc4c(ccc5ccc6ccc7nc(-c8ccccc8)oc7c6c54)c3)n2)cc1 ; c1ccc(-c2ccc(-c3ccc(-c4nc(-c5ccccc5)nc(-c5ccc6c(ccc7ccc8ccc9nc(-c%10ccccc%10)oc9c8c76)c5)n4)cc3)cc2)cc1 ; c1ccc(-c2cccc(-c3ccc(-c4nc(-c5ccccc5)nc(-c5ccc6c(ccc7ccc8ccc9nc(-c%10ccccc%10)oc9c8c76)c5)n4)cc3)c2)cc1]

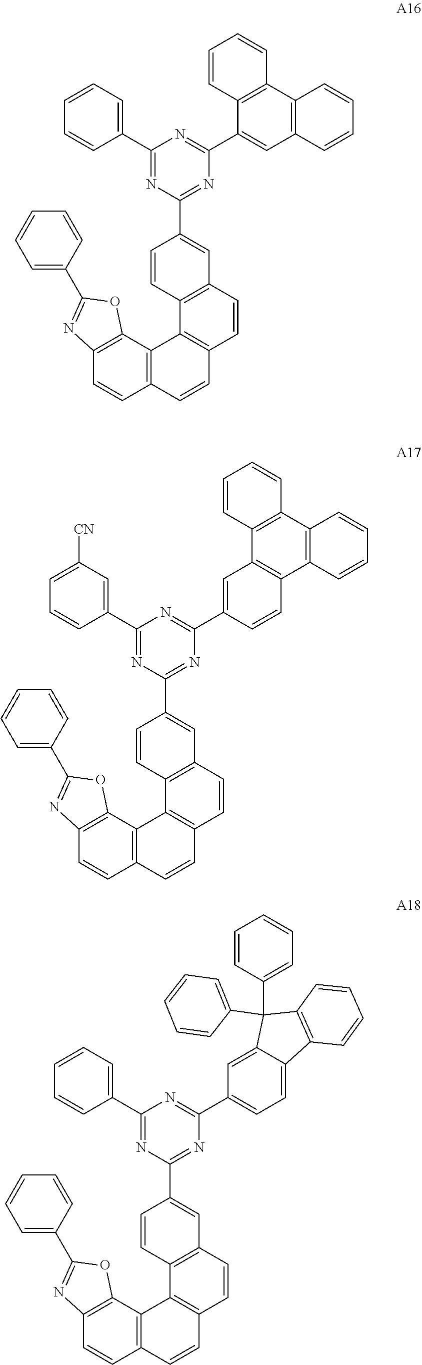 [SMILES: c1ccc(-c2nc(-c3ccc4c(ccc5ccc6ccc7nc(-c8ccccc8)oc7c6c54)c3)nc(-c3cc4ccccc4c4ccccc34)n2)cc1 ; N#Cc1cccc(-c2nc(-c3ccc4c(ccc5ccc6ccc7nc(-c8ccccc8)oc7c6c54)c3)nc(-c3ccc4c5ccccc5c5ccccc5c4c3)n2)c1 ; c1ccc(-c2nc(-c3ccc4c(c3)C(c3ccccc3)(c3ccccc3)c3ccccc3-4)nc(-c3ccc4c(ccc5ccc6ccc7nc(-c8ccccc8)oc7c6c54)c3)n2)cc1]

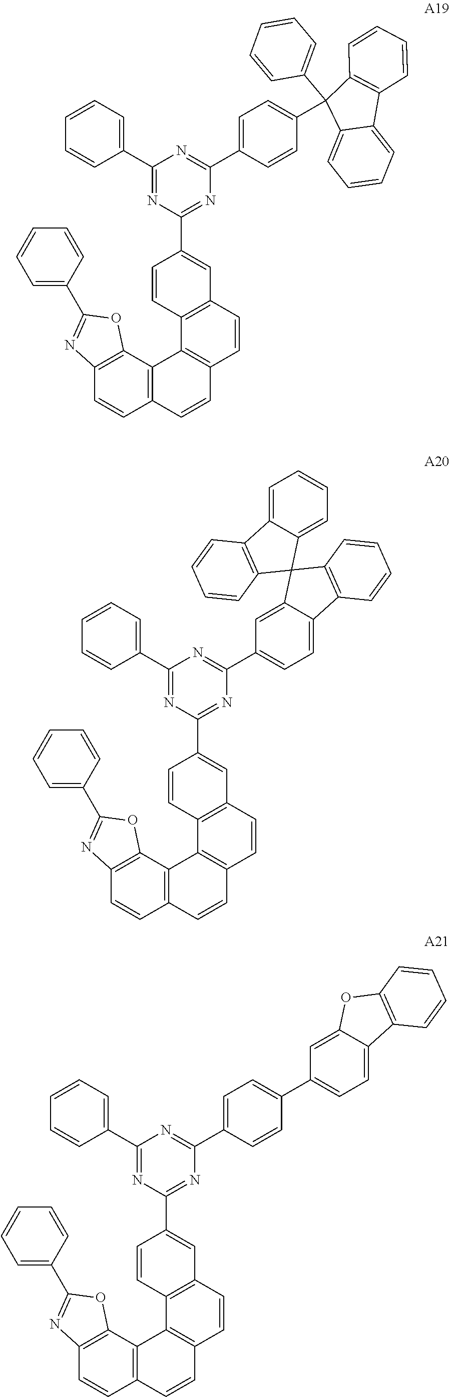 [SMILES: c1ccc(-c2nc(-c3ccc(C4(c5ccccc5)c5ccccc5-c5ccccc54)cc3)nc(-c3ccc4c(ccc5ccc6ccc7nc(-c8ccccc8)oc7c6c54)c3)n2)cc1 ; c1ccc(-c2nc(-c3ccc4c(c3)C3(c5ccccc5-c5ccccc53)c3ccccc3-4)nc(-c3ccc4c(ccc5ccc6ccc7nc(-c8ccccc8)oc7c6c54)c3)n2)cc1 ; c1ccc(-c2nc(-c3ccc(-c4ccc5c(c4)oc4ccccc45)cc3)nc(-c3ccc4c(ccc5ccc6ccc7nc(-c8ccccc8)oc7c6c54)c3)n2)cc1]

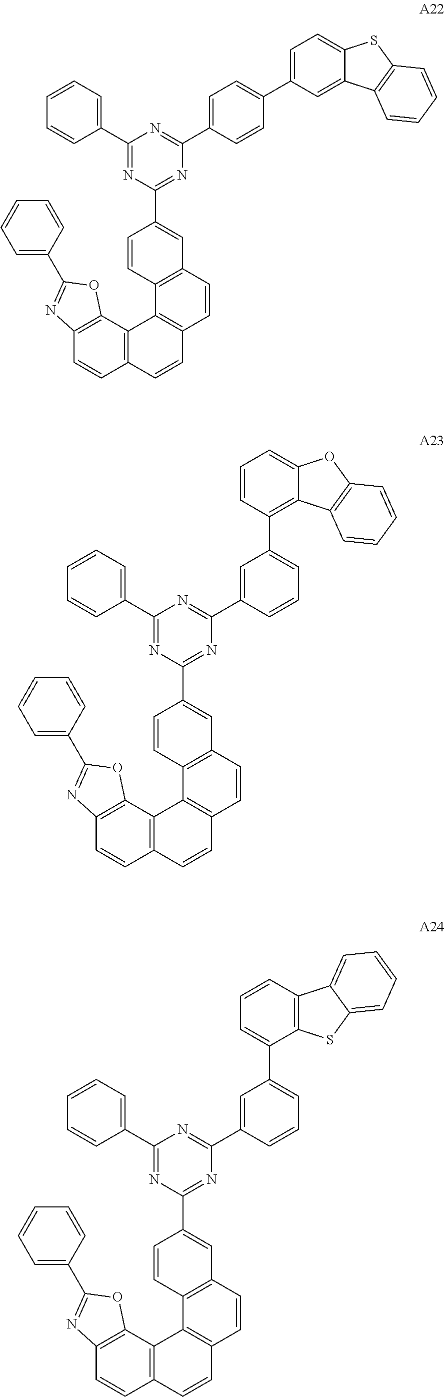 [SMILES: c1ccc(-c2nc(-c3ccc(-c4ccc5sc6ccccc6c5c4)cc3)nc(-c3ccc4c(ccc5ccc6ccc7nc(-c8ccccc8)oc7c6c54)c3)n2)cc1 ; c1ccc(-c2nc(-c3cccc(-c4cccc5oc6ccccc6c45)c3)nc(-c3ccc4c(ccc5ccc6ccc7nc(-c8ccccc8)oc7c6c54)c3)n2)cc1 ; c1ccc(-c2nc(-c3cccc(-c4cccc5c4sc4ccccc45)c3)nc(-c3ccc4c(ccc5ccc6ccc7nc(-c8ccccc8)oc7c6c54)c3)n2)cc1]

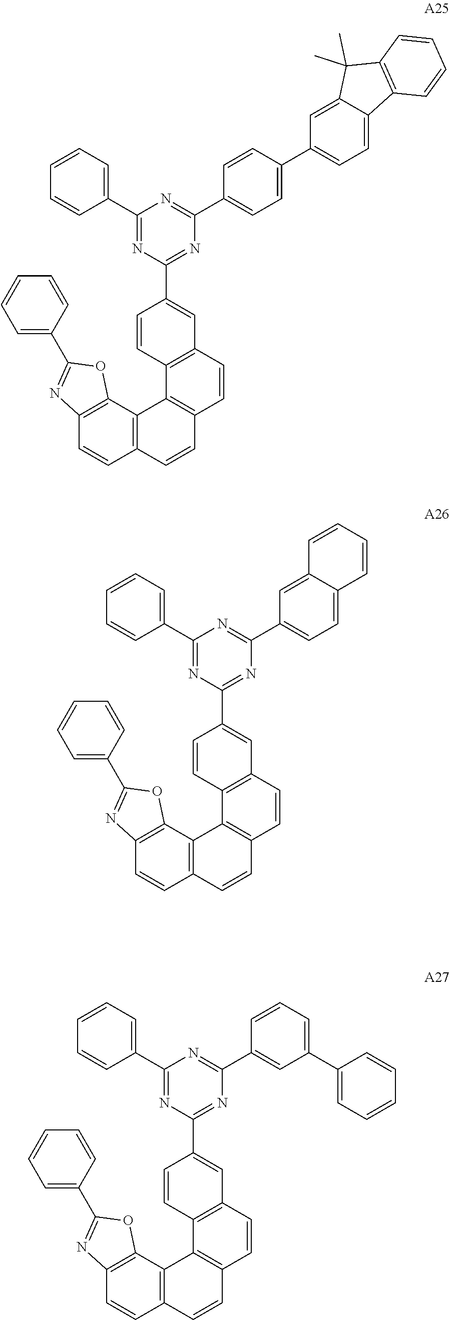 [SMILES: CC1(C)c2ccccc2-c2ccc(-c3ccc(-c4nc(-c5ccccc5)nc(-c5ccc6c(ccc7ccc8ccc9nc(-c%10ccccc%10)oc9c8c76)c5)n4)cc3)cc21 ; c1ccc(-c2nc(-c3ccc4ccccc4c3)nc(-c3ccc4c(ccc5ccc6ccc7nc(-c8ccccc8)oc7c6c54)c3)n2)cc1 ; c1ccc(-c2cccc(-c3nc(-c4ccccc4)nc(-c4ccc5c(ccc6ccc7ccc8nc(-c9ccccc9)oc8c7c65)c4)n3)c2)cc1]

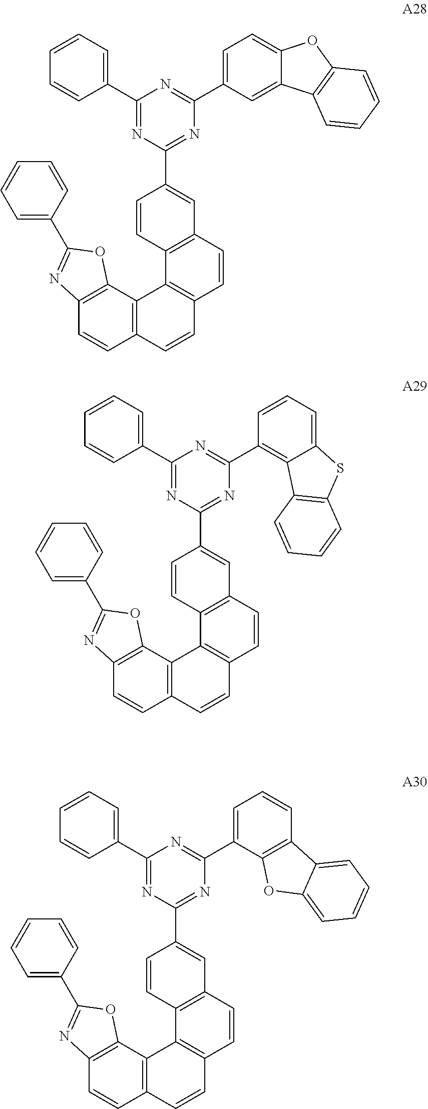

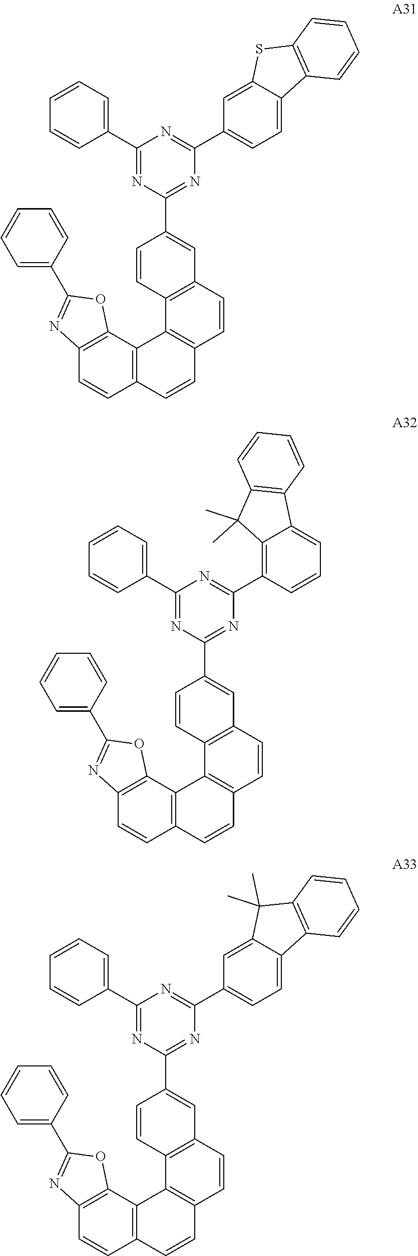 [SMILES: c1ccc(-c2nc(-c3ccc4c(ccc5ccc6ccc7nc(-c8ccccc8)oc7c6c54)c3)nc(-c3ccc4c(c3)sc3ccccc34)n2)cc1 ; CC1(C)c2ccccc2-c2cccc(-c3nc(-c4ccccc4)nc(-c4ccc5c(ccc6ccc7ccc8nc(-c9ccccc9)oc8c7c65)c4)n3)c21 ; CC1(C)c2ccccc2-c2ccc(-c3nc(-c4ccccc4)nc(-c4ccc5c(ccc6ccc7ccc8nc(-c9ccccc9)oc8c7c65)c4)n3)cc21]

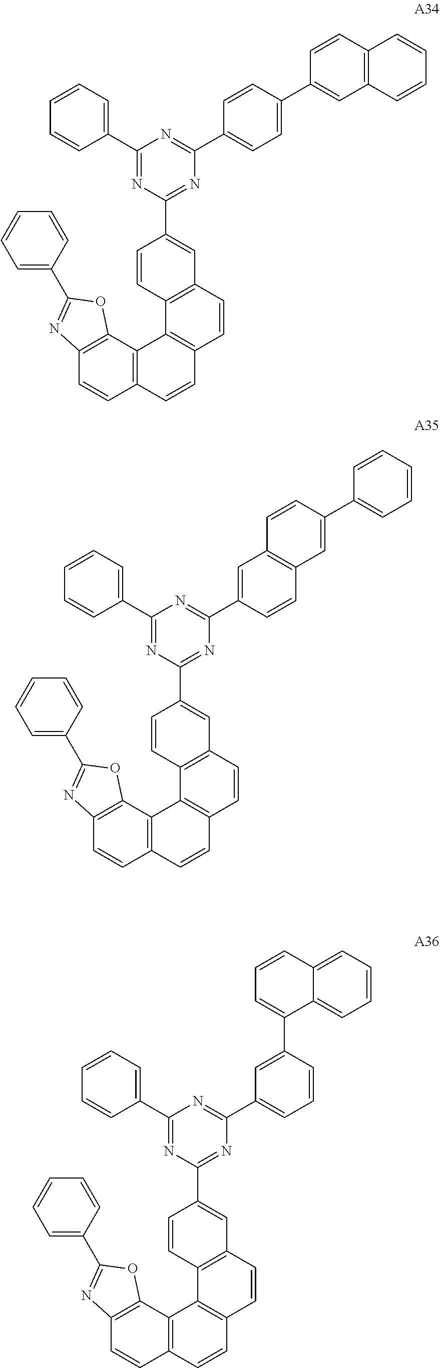 [SMILES: c1ccc(-c2nc(-c3ccc(-c4ccc5ccccc5c4)cc3)nc(-c3ccc4c(ccc5ccc6ccc7nc(-c8ccccc8)oc7c6c54)c3)n2)cc1 ; c1ccc(-c2ccc3cc(-c4nc(-c5ccccc5)nc(-c5ccc6c(ccc7ccc8ccc9nc(-c%10ccccc%10)oc9c8c76)c5)n4)ccc3c2)cc1 ; c1ccc(-c2nc(-c3cccc(-c4cccc5ccccc45)c3)nc(-c3ccc4c(ccc5ccc6ccc7nc(-c8ccccc8)oc7c6c54)c3)n2)cc1]

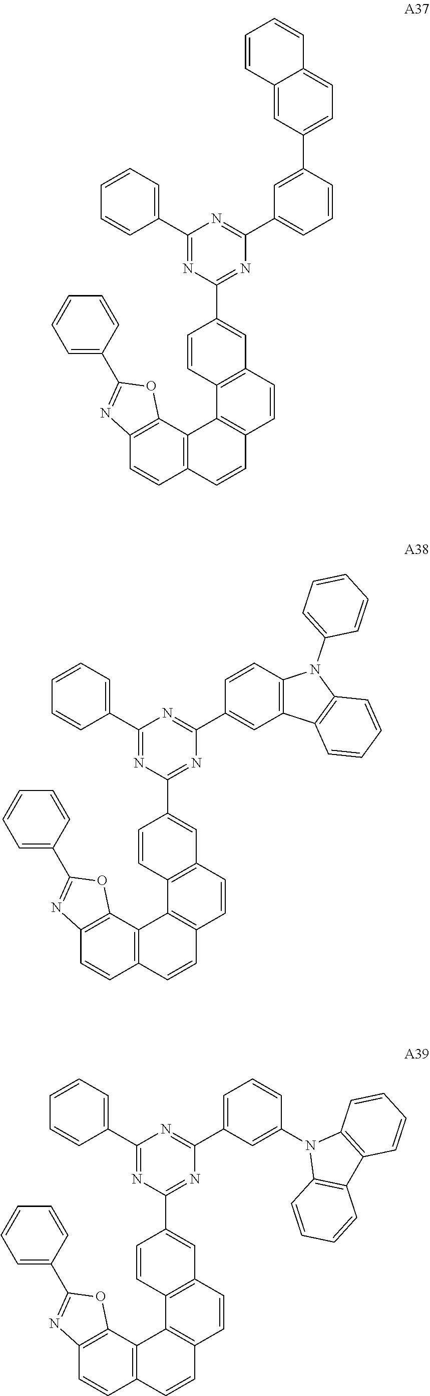 [SMILES: c1ccc(-c2nc(-c3cccc(-c4ccc5ccccc5c4)c3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3ccc4c(c3)c3ccccc3n4-c3ccccc3)n2)cc1 ; c1ccc(-c2nc(-c3cccc(-n4c5ccccc5c5ccccc54)c3)nc(-c3ccc4c(ccc5ccc6ccc7nc(-c8ccccc8)oc7c6c54)c3)n2)cc1]

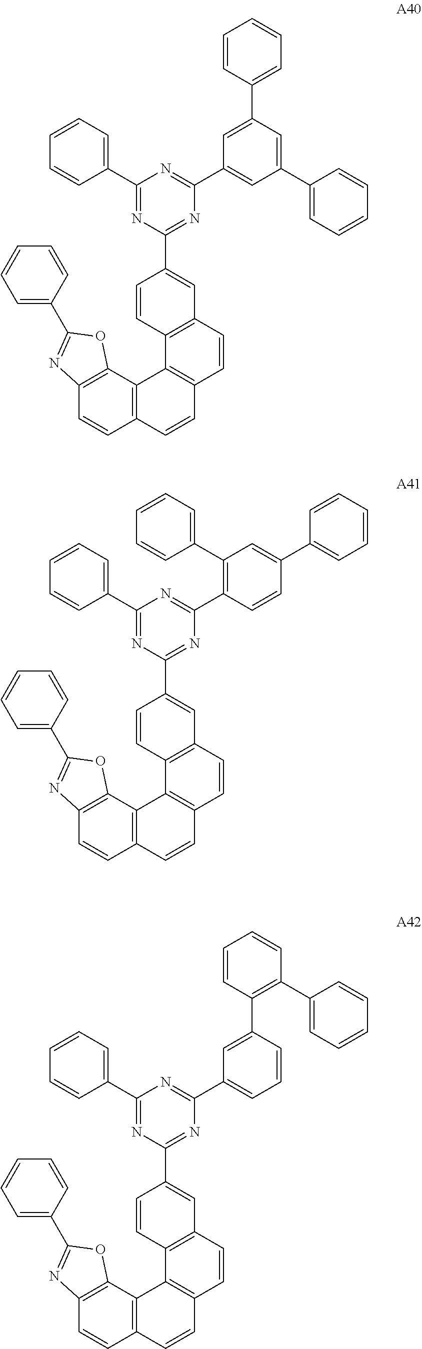 [SMILES: c1ccc(-c2cc(-c3ccccc3)cc(-c3nc(-c4ccccc4)nc(-c4ccc5c(ccc6ccc7ccc8nc(-c9ccccc9)oc8c7c65)c4)n3)c2)cc1 ; c1ccc(-c2ccc(-c3nc(-c4ccccc4)nc(-c4ccc5c(ccc6ccc7ccc8nc(-c9ccccc9)oc8c7c65)c4)n3)c(-c3ccccc3)c2)cc1 ; c1ccc(-c2nc(-c3cccc(-c4ccccc4-c4ccccc4)c3)nc(-c3ccc4c(ccc5ccc6ccc7nc(-c8ccccc8)oc7c6c54)c3)n2)cc1]

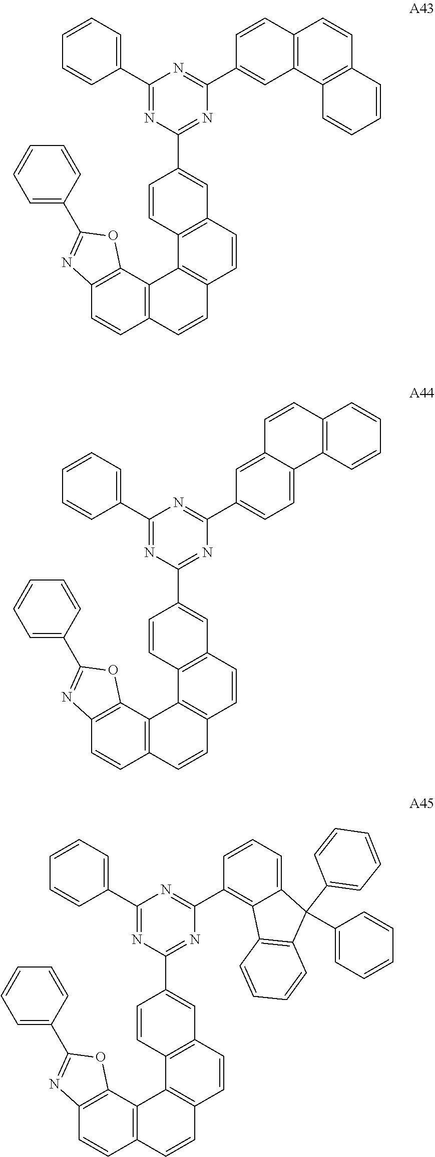 [SMILES: c1ccc(-c2nc(-c3ccc4c(ccc5ccc6ccc7nc(-c8ccccc8)oc7c6c54)c3)nc(-c3ccc4ccc5ccccc5c4c3)n2)cc1 ; c1ccc(-c2nc(-c3ccc4c(ccc5ccccc54)c3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3cccc4c3-c3ccccc3C4(c3ccccc3)c3ccccc3)n2)cc1]

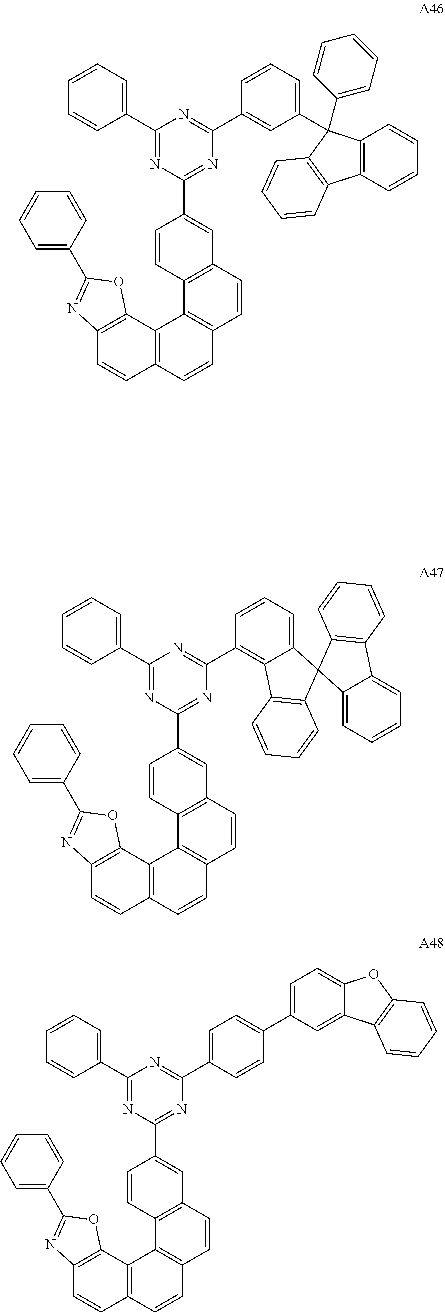 [SMILES: c1ccc(-c2nc(-c3cccc(C4(c5ccccc5)c5ccccc5-c5ccccc54)c3)nc(-c3ccc4c(ccc5ccc6ccc7nc(-c8ccccc8)oc7c6c54)c3)n2)cc1 ; c1ccc(-c2nc(-c3ccc4c(ccc5ccc6ccc7nc(-c8ccccc8)oc7c6c54)c3)nc(-c3cccc4c3-c3ccccc3C43c4ccccc4-c4ccccc43)n2)cc1 ; c1ccc(-c2nc(-c3ccc(-c4ccc5oc6ccccc6c5c4)cc3)nc(-c3ccc4c(ccc5ccc6ccc7nc(-c8ccccc8)oc7c6c54)c3)n2)cc1]

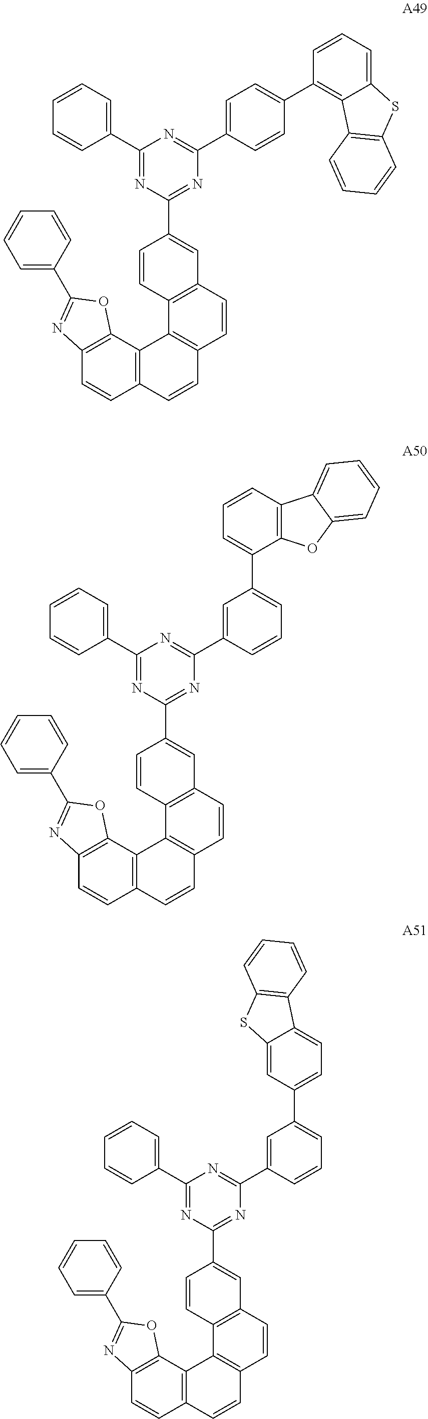 [SMILES: c1ccc(-c2nc(-c3ccc(-c4cccc5sc6ccccc6c45)cc3)nc(-c3ccc4c(ccc5ccc6ccc7nc(-c8ccccc8)oc7c6c54)c3)n2)cc1 ; c1ccc(-c2nc(-c3cccc(-c4cccc5c4oc4ccccc45)c3)nc(-c3ccc4c(ccc5ccc6ccc7nc(-c8ccccc8)oc7c6c54)c3)n2)cc1 ; c1ccc(-c2nc(-c3cccc(-c4ccc5c(c4)sc4ccccc45)c3)nc(-c3ccc4c(ccc5ccc6ccc7nc(-c8ccccc8)oc7c6c54)c3)n2)cc1]

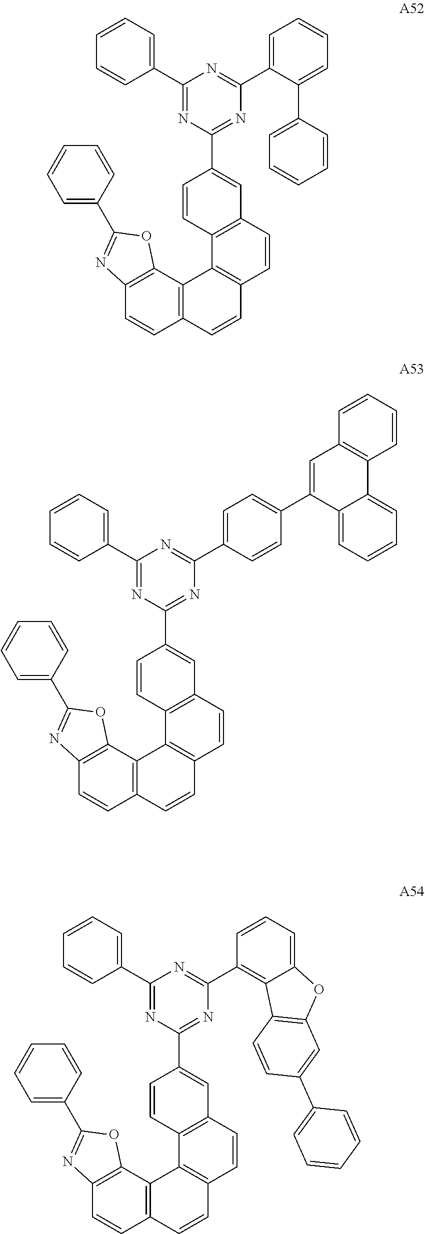 [SMILES: c1ccc(-c2nc(-c3ccc4c(ccc5ccc6ccc7nc(-c8ccccc8)oc7c6c54)c3)nc(-c3ccccc3-c3ccccc3)n2)cc1 ; c1ccc(-c2nc(-c3ccc(-c4cc5ccccc5c5ccccc45)cc3)nc(-c3ccc4c(ccc5ccc6ccc7nc(-c8ccccc8)oc7c6c54)c3)n2)cc1 ; c1ccc(-c2ccc3c(c2)oc2cccc(-c4nc(-c5ccccc5)nc(-c5ccc6c(ccc7ccc8ccc9nc(-c%10ccccc%10)oc9c8c76)c5)n4)c23)cc1]

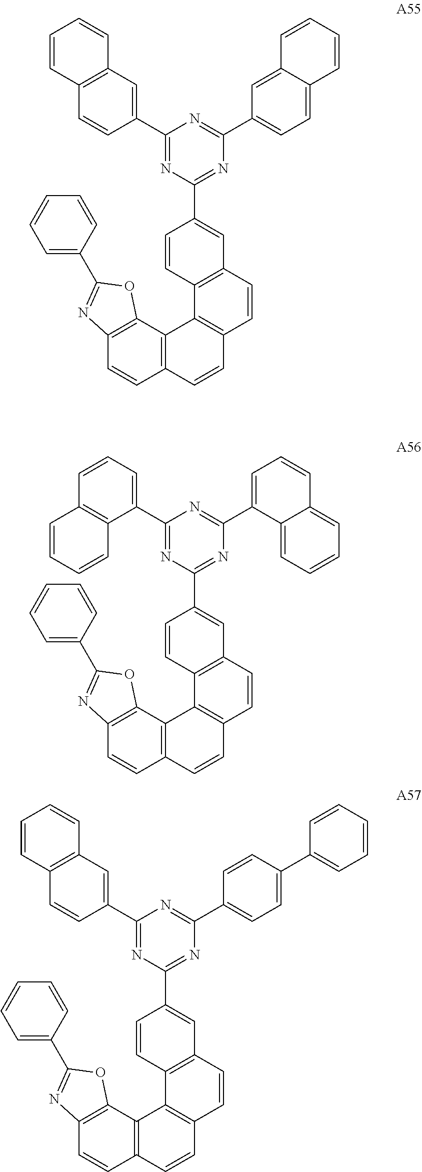 [SMILES: c1ccc(-c2nc3ccc4ccc5ccc6cc(-c7nc(-c8ccc9ccccc9c8)nc(-c8ccc9ccccc9c8)n7)ccc6c5c4c3o2)cc1 ; c1ccc(-c2nc3ccc4ccc5ccc6cc(-c7nc(-c8cccc9ccccc89)nc(-c8cccc9ccccc89)n7)ccc6c5c4c3o2)cc1 ; c1ccc(-c2ccc(-c3nc(-c4ccc5ccccc5c4)nc(-c4ccc5c(ccc6ccc7ccc8nc(-c9ccccc9)oc8c7c65)c4)n3)cc2)cc1]

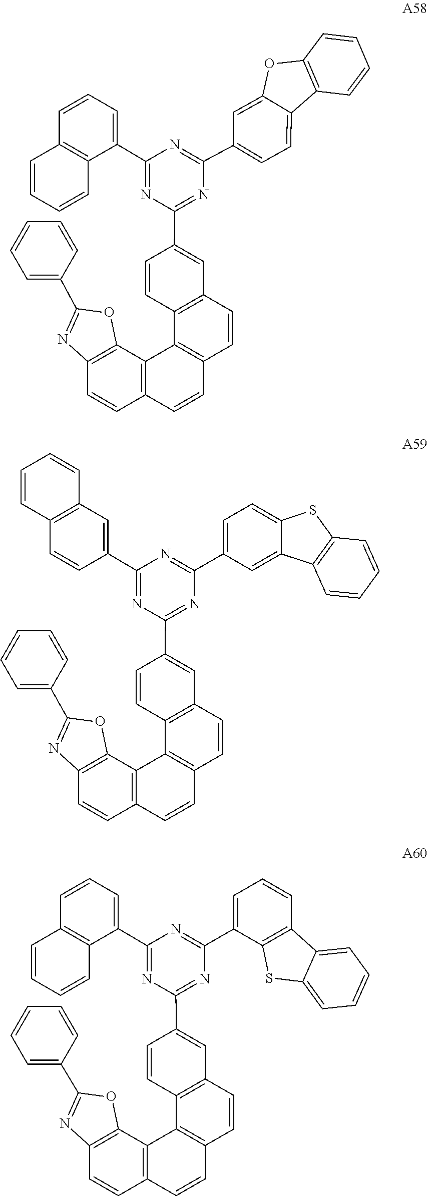 [SMILES: c1ccc(-c2nc3ccc4ccc5ccc6cc(-c7nc(-c8ccc9c(c8)oc8ccccc89)nc(-c8cccc9ccccc89)n7)ccc6c5c4c3o2)cc1 ; c1ccc(-c2nc3ccc4ccc5ccc6cc(-c7nc(-c8ccc9ccccc9c8)nc(-c8ccc9sc%10ccccc%10c9c8)n7)ccc6c5c4c3o2)cc1 ; c1ccc(-c2nc3ccc4ccc5ccc6cc(-c7nc(-c8cccc9ccccc89)nc(-c8cccc9c8sc8ccccc89)n7)ccc6c5c4c3o2)cc1]

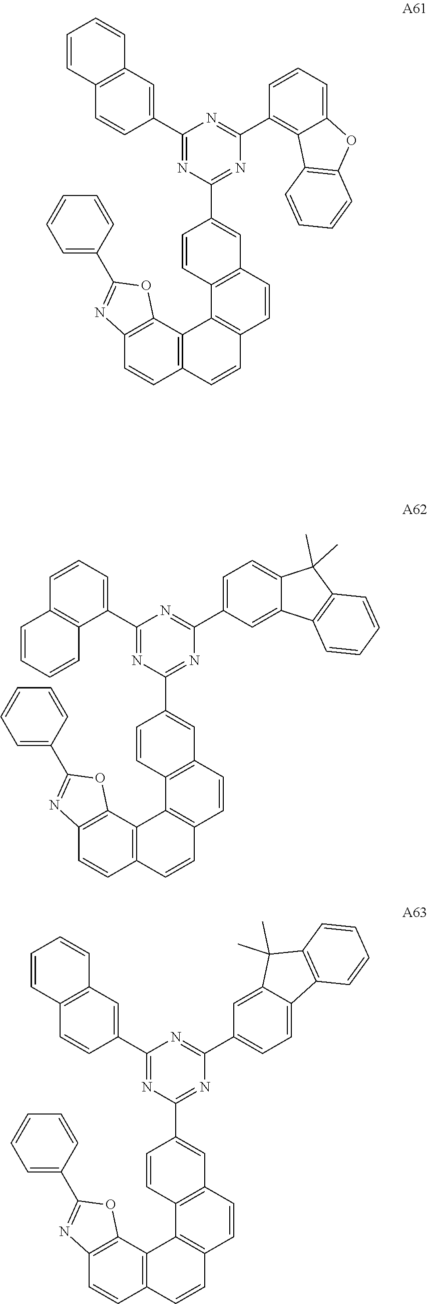 [SMILES: c1ccc(-c2nc3ccc4ccc5ccc6cc(-c7nc(-c8ccc9ccccc9c8)nc(-c8cccc9oc%10ccccc%10c89)n7)ccc6c5c4c3o2)cc1 ; CC1(C)c2ccccc2-c2cc(-c3nc(-c4ccc5c(ccc6ccc7ccc8nc(-c9ccccc9)oc8c7c65)c4)nc(-c4cccc5ccccc45)n3)ccc21 ; CC1(C)c2ccccc2-c2ccc(-c3nc(-c4ccc5ccccc5c4)nc(-c4ccc5c(ccc6ccc7ccc8nc(-c9ccccc9)oc8c7c65)c4)n3)cc21]

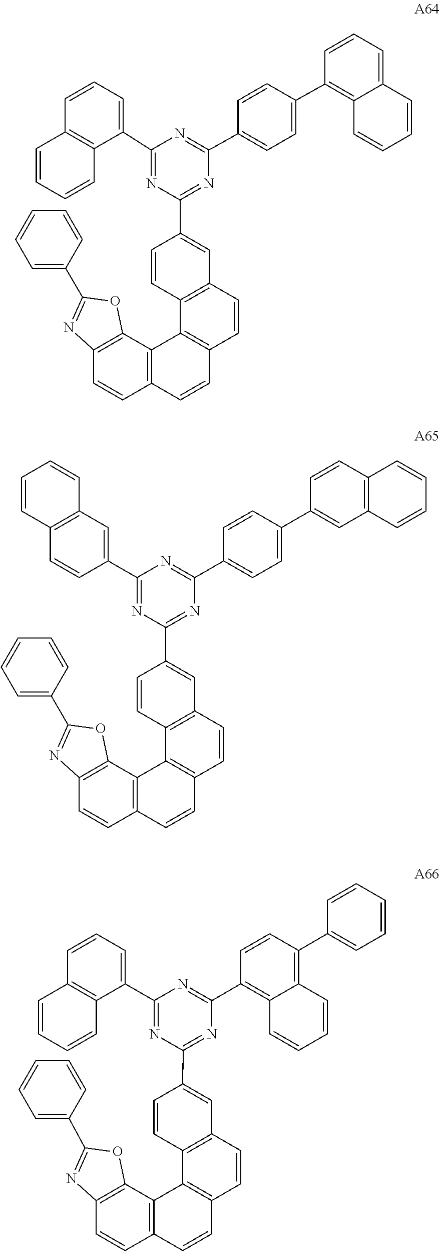 [SMILES: c1ccc(-c2nc3ccc4ccc5ccc6cc(-c7nc(-c8ccc(-c9cccc%10ccccc9%10)cc8)nc(-c8cccc9ccccc89)n7)ccc6c5c4c3o2)cc1 ; c1ccc(-c2nc3ccc4ccc5ccc6cc(-c7nc(-c8ccc(-c9ccc%10ccccc%10c9)cc8)nc(-c8ccc9ccccc9c8)n7)ccc6c5c4c3o2)cc1 ; c1ccc(-c2nc3ccc4ccc5ccc6cc(-c7nc(-c8cccc9ccccc89)nc(-c8ccc(-c9ccccc9)c9ccccc89)n7)ccc6c5c4c3o2)cc1]

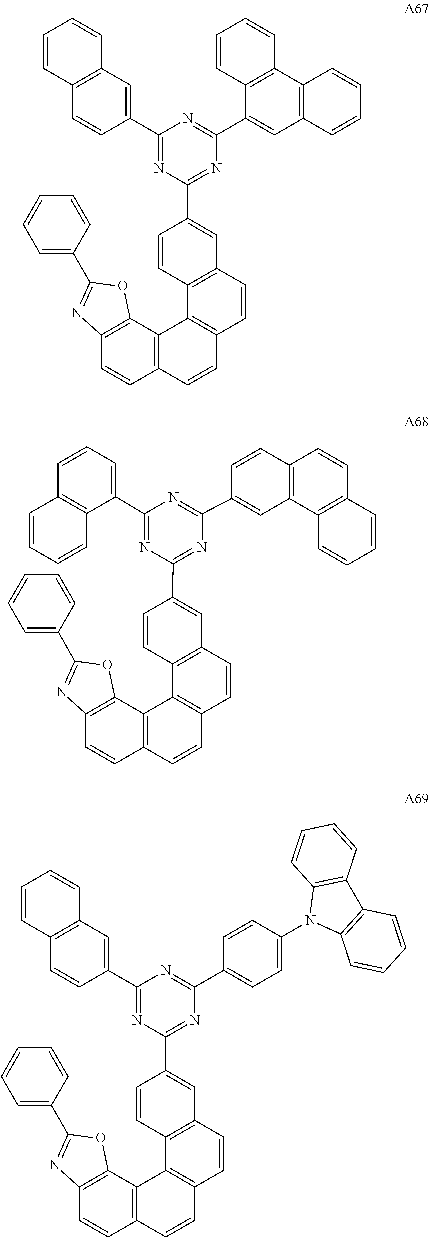 [SMILES: c1ccc(-c2nc3ccc4ccc5ccc6cc(-c7nc(-c8ccc9ccccc9c8)nc(-c8cc9ccccc9c9ccccc89)n7)ccc6c5c4c3o2)cc1 ; c1ccc(-c2nc3ccc4ccc5ccc6cc(-c7nc(-c8ccc9ccc%10ccccc%10c9c8)nc(-c8cccc9ccccc89)n7)ccc6c5c4c3o2)cc1 ; c1ccc(-c2nc3ccc4ccc5ccc6cc(-c7nc(-c8ccc(-n9c%10ccccc%10c%10ccccc%109)cc8)nc(-c8ccc9ccccc9c8)n7)ccc6c5c4c3o2)cc1]

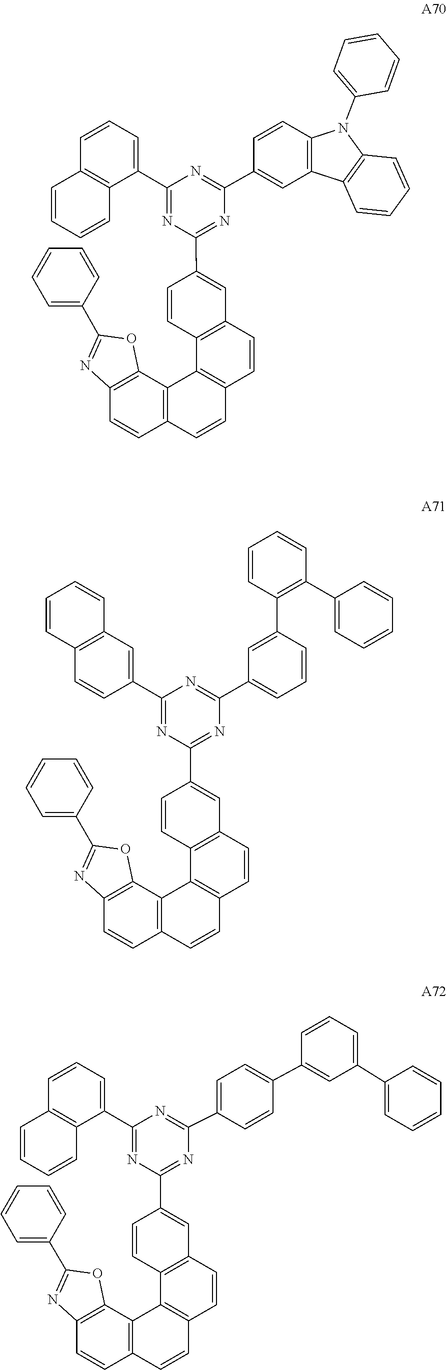 [SMILES: c1ccc(-c2nc3ccc4ccc5ccc6cc(-c7nc(-c8ccc9c(c8)c8ccccc8n9-c8ccccc8)nc(-c8cccc9ccccc89)n7)ccc6c5c4c3o2)cc1 ; c1ccc(-c2nc3ccc4ccc5ccc6cc(-c7nc(-c8cccc(-c9ccccc9-c9ccccc9)c8)nc(-c8ccc9ccccc9c8)n7)ccc6c5c4c3o2)cc1 ; c1ccc(-c2cccc(-c3ccc(-c4nc(-c5ccc6c(ccc7ccc8ccc9nc(-c%10ccccc%10)oc9c8c76)c5)nc(-c5cccc6ccccc56)n4)cc3)c2)cc1]

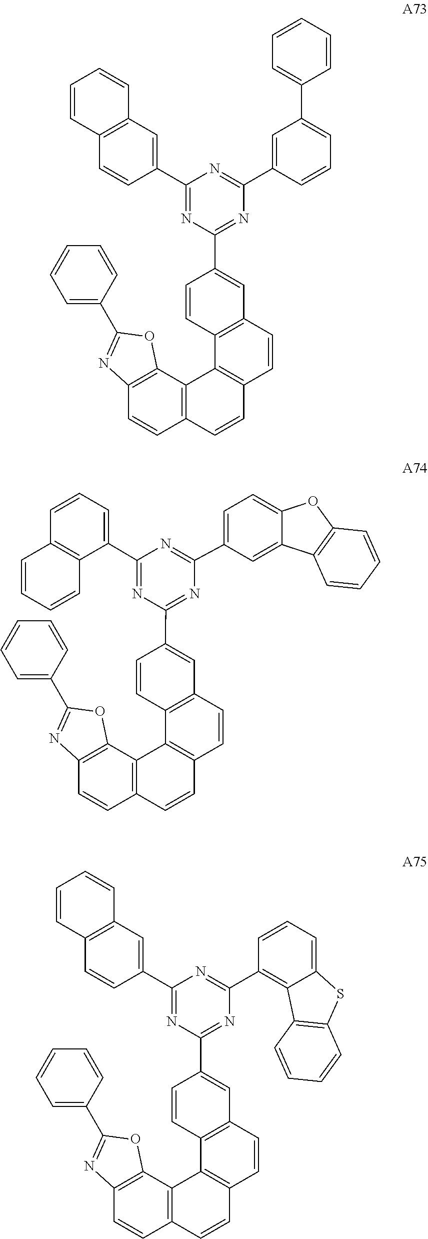 [SMILES: c1ccc(-c2cccc(-c3nc(-c4ccc5ccccc5c4)nc(-c4ccc5c(ccc6ccc7ccc8nc(-c9ccccc9)oc8c7c65)c4)n3)c2)cc1 ; c1ccc(-c2nc3ccc4ccc5ccc6cc(-c7nc(-c8ccc9oc%10ccccc%10c9c8)nc(-c8cccc9ccccc89)n7)ccc6c5c4c3o2)cc1 ; c1ccc(-c2nc3ccc4ccc5ccc6cc(-c7nc(-c8ccc9ccccc9c8)nc(-c8cccc9sc%10ccccc%10c89)n7)ccc6c5c4c3o2)cc1]

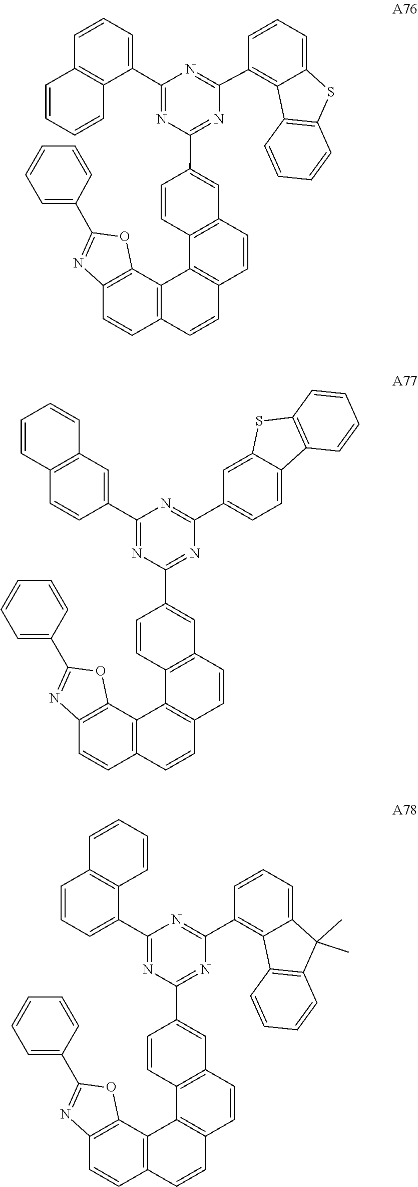 [SMILES: c1ccc(-c2nc3ccc4ccc5ccc6cc(-c7nc(-c8cccc9ccccc89)nc(-c8cccc9sc%10ccccc%10c89)n7)ccc6c5c4c3o2)cc1 ; c1ccc(-c2nc3ccc4ccc5ccc6cc(-c7nc(-c8ccc9ccccc9c8)nc(-c8ccc9c(c8)sc8ccccc89)n7)ccc6c5c4c3o2)cc1 ; CC1(C)c2ccccc2-c2c(-c3nc(-c4ccc5c(ccc6ccc7ccc8nc(-c9ccccc9)oc8c7c65)c4)nc(-c4cccc5ccccc45)n3)cccc21]

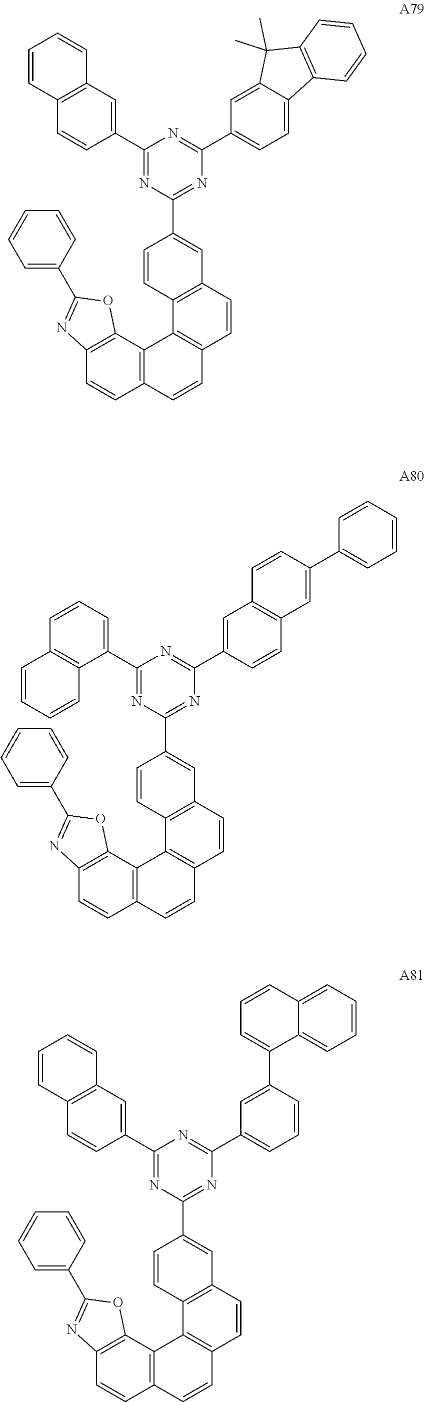 [SMILES: CC1(C)c2ccccc2-c2ccc(-c3nc(-c4ccc5ccccc5c4)nc(-c4ccc5c(ccc6ccc7ccc8nc(-c9ccccc9)oc8c7c65)c4)n3)cc21 ; c1ccc(-c2ccc3cc(-c4nc(-c5ccc6c(ccc7ccc8ccc9nc(-c%10ccccc%10)oc9c8c76)c5)nc(-c5cccc6ccccc56)n4)ccc3c2)cc1 ; c1ccc(-c2nc3ccc4ccc5ccc6cc(-c7nc(-c8cccc(-c9cccc%10ccccc9%10)c8)nc(-c8ccc9ccccc9c8)n7)ccc6c5c4c3o2)cc1]

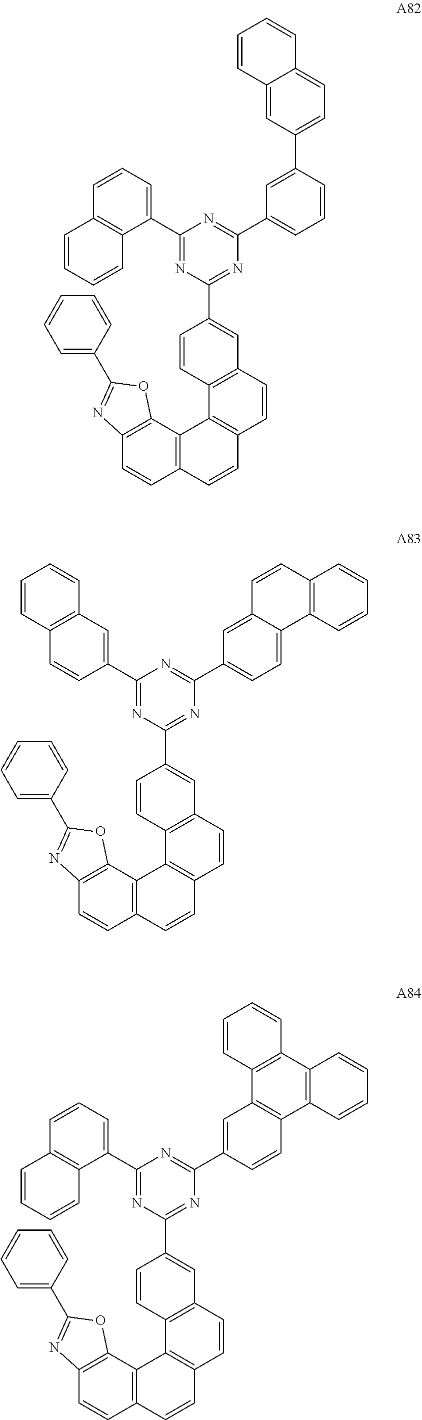 [SMILES: c1ccc(-c2nc3ccc4ccc5ccc6cc(-c7nc(-c8cccc(-c9ccc%10ccccc%10c9)c8)nc(-c8cccc9ccccc89)n7)ccc6c5c4c3o2)cc1 ; c1ccc(-c2nc3ccc4ccc5ccc6cc(-c7nc(-c8ccc9ccccc9c8)nc(-c8ccc9c(ccc%10ccccc%109)c8)n7)ccc6c5c4c3o2)cc1 ; c1ccc(-c2nc3ccc4ccc5ccc6cc(-c7nc(-c8ccc9c%10ccccc%10c%10ccccc%10c9c8)nc(-c8cccc9ccccc89)n7)ccc6c5c4c3o2)cc1]

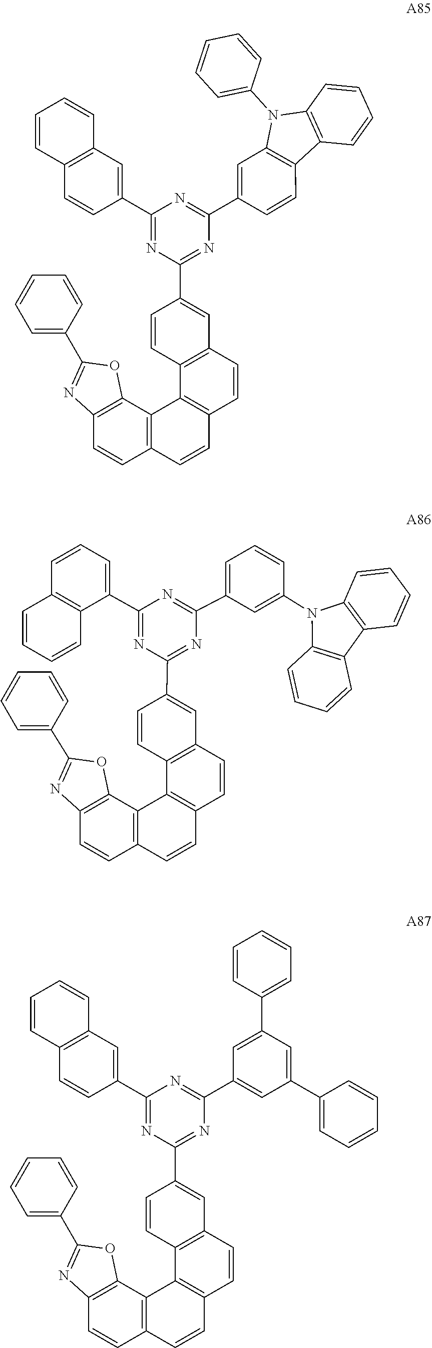 [SMILES: c1ccc(-c2nc3ccc4ccc5ccc6cc(-c7nc(-c8ccc9ccccc9c8)nc(-c8ccc9c%10ccccc%10n(-c%10ccccc%10)c9c8)n7)ccc6c5c4c3o2)cc1 ; c1ccc(-c2nc3ccc4ccc5ccc6cc(-c7nc(-c8cccc(-n9c%10ccccc%10c%10ccccc%109)c8)nc(-c8cccc9ccccc89)n7)ccc6c5c4c3o2)cc1 ; c1ccc(-c2cc(-c3ccccc3)cc(-c3nc(-c4ccc5ccccc5c4)nc(-c4ccc5c(ccc6ccc7ccc8nc(-c9ccccc9)oc8c7c65)c4)n3)c2)cc1]

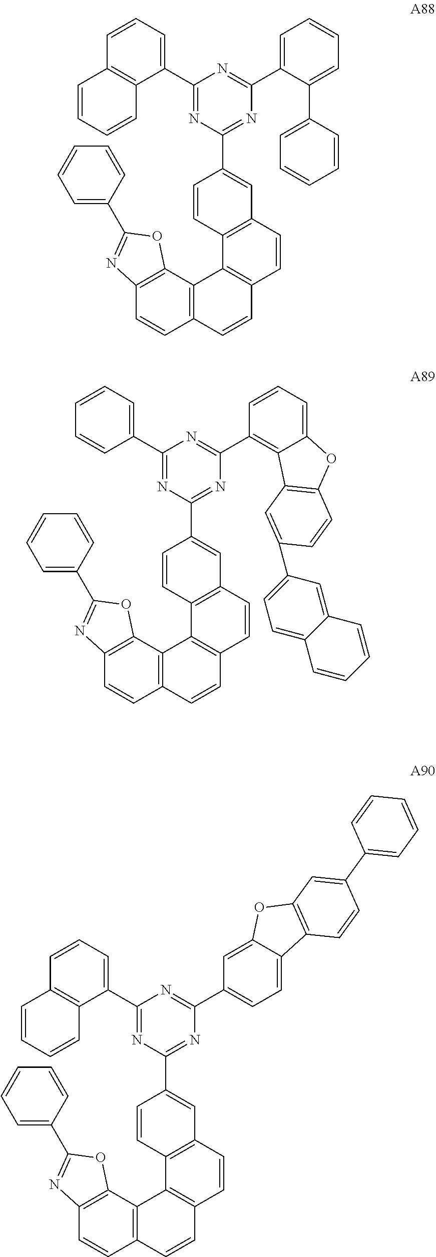 [SMILES: c1ccc(-c2nc3ccc4ccc5ccc6cc(-c7nc(-c8ccccc8-c8ccccc8)nc(-c8cccc9ccccc89)n7)ccc6c5c4c3o2)cc1 ; c1ccc(-c2nc(-c3ccc4c(ccc5ccc6ccc7nc(-c8ccccc8)oc7c6c54)c3)nc(-c3cccc4oc5ccc(-c6ccc7ccccc7c6)cc5c34)n2)cc1 ; c1ccc(-c2ccc3c(c2)oc2cc(-c4nc(-c5ccc6c(ccc7ccc8ccc9nc(-c%10ccccc%10)oc9c8c76)c5)nc(-c5cccc6ccccc56)n4)ccc23)cc1]

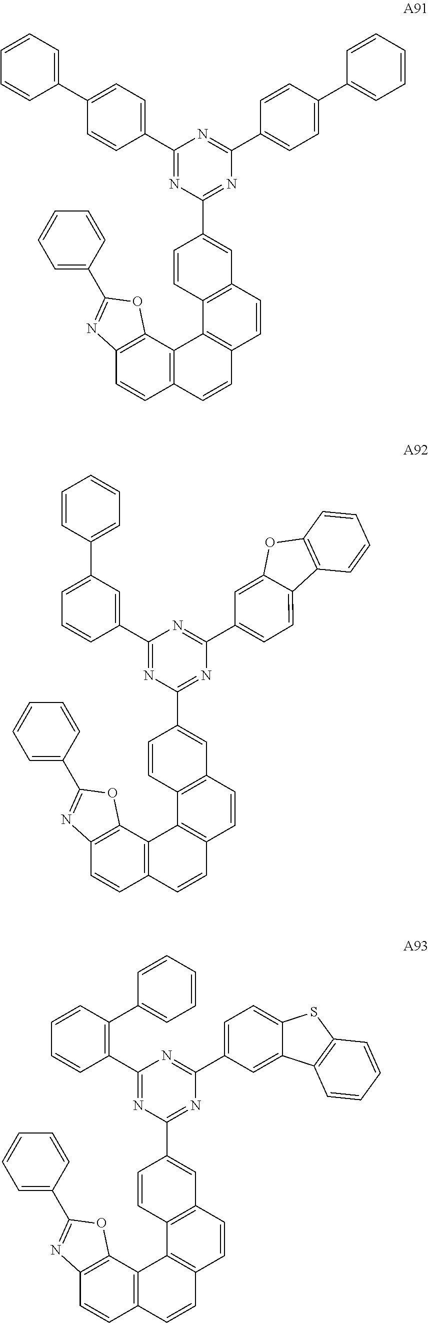 [SMILES: c1ccc(-c2ccc(-c3nc(-c4ccc(-c5ccccc5)cc4)nc(-c4ccc5c(ccc6ccc7ccc8nc(-c9ccccc9)oc8c7c65)c4)n3)cc2)cc1 ; c1ccc(-c2cccc(-c3nc(-c4ccc5c(ccc6ccc7ccc8nc(-c9ccccc9)oc8c7c65)c4)nc(-c4ccc5c(c4)oc4ccccc45)n3)c2)cc1 ; c1ccc(-c2nc3ccc4ccc5ccc6cc(-c7nc(-c8ccc9sc%10ccccc%10c9c8)nc(-c8ccccc8-c8ccccc8)n7)ccc6c5c4c3o2)cc1]

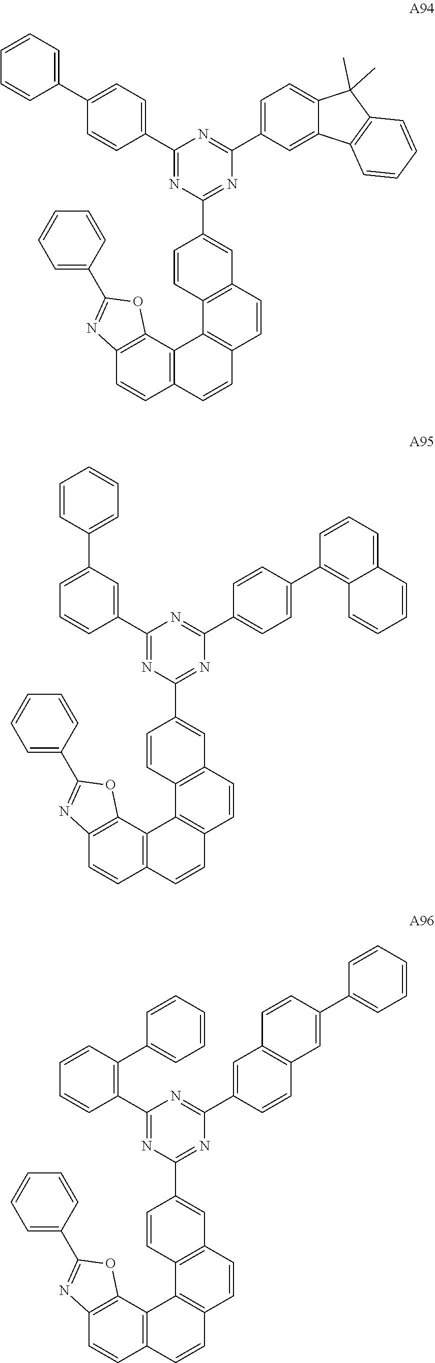 [SMILES: CC1(C)c2ccccc2-c2cc(-c3nc(-c4ccc(-c5ccccc5)cc4)nc(-c4ccc5c(ccc6ccc7ccc8nc(-c9ccccc9)oc8c7c65)c4)n3)ccc21 ; c1ccc(-c2cccc(-c3nc(-c4ccc(-c5cccc6ccccc56)cc4)nc(-c4ccc5c(ccc6ccc7ccc8nc(-c9ccccc9)oc8c7c65)c4)n3)c2)cc1 ; c1ccc(-c2ccc3cc(-c4nc(-c5ccc6c(ccc7ccc8ccc9nc(-c%10ccccc%10)oc9c8c76)c5)nc(-c5ccccc5-c5ccccc5)n4)ccc3c2)cc1]

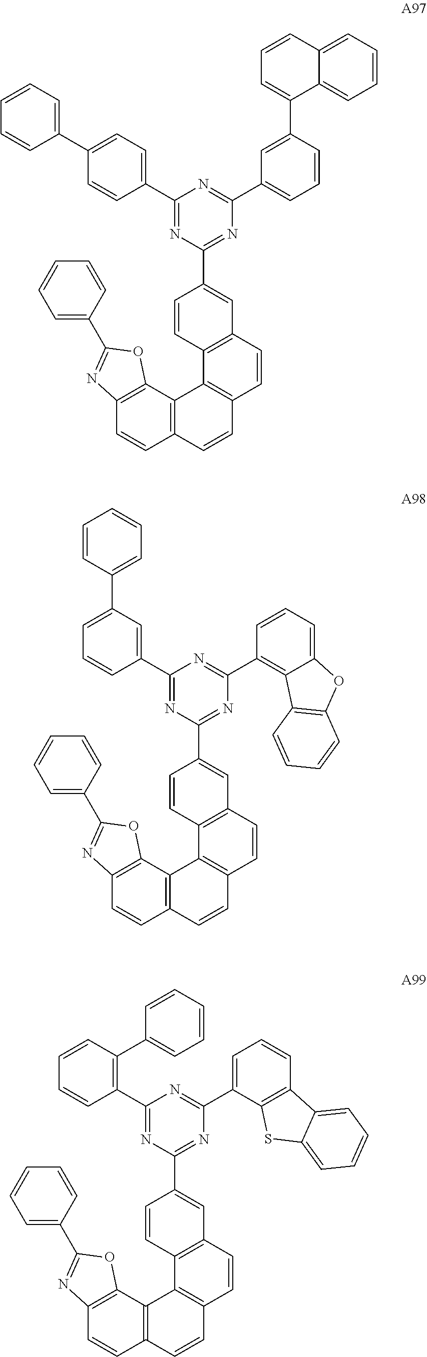 [SMILES: c1ccc(-c2ccc(-c3nc(-c4cccc(-c5cccc6ccccc56)c4)nc(-c4ccc5c(ccc6ccc7ccc8nc(-c9ccccc9)oc8c7c65)c4)n3)cc2)cc1 ; c1ccc(-c2cccc(-c3nc(-c4ccc5c(ccc6ccc7ccc8nc(-c9ccccc9)oc8c7c65)c4)nc(-c4cccc5oc6ccccc6c45)n3)c2)cc1 ; c1ccc(-c2nc3ccc4ccc5ccc6cc(-c7nc(-c8ccccc8-c8ccccc8)nc(-c8cccc9c8sc8ccccc89)n7)ccc6c5c4c3o2)cc1]

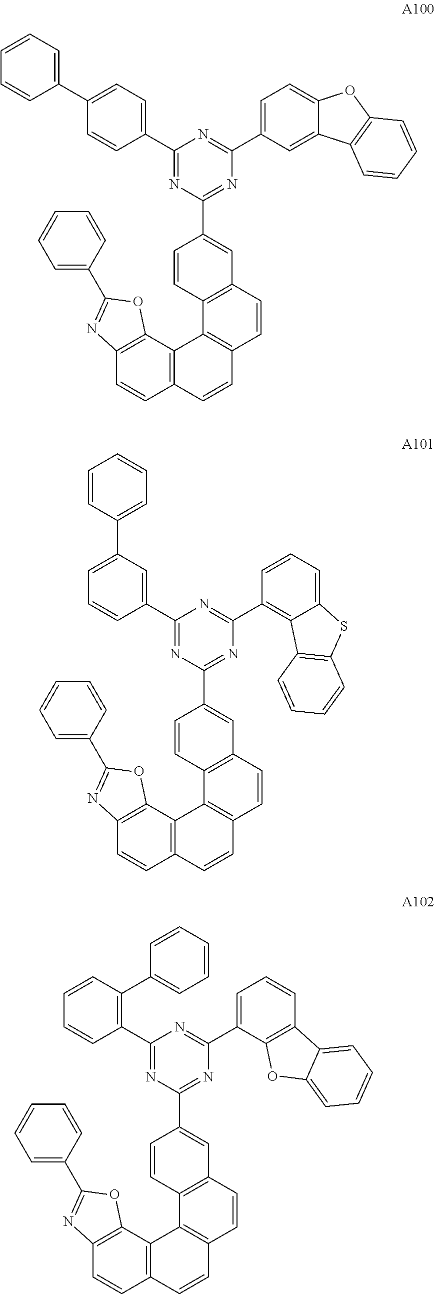 [SMILES: c1ccc(-c2ccc(-c3nc(-c4ccc5c(ccc6ccc7ccc8nc(-c9ccccc9)oc8c7c65)c4)nc(-c4ccc5oc6ccccc6c5c4)n3)cc2)cc1 ; c1ccc(-c2cccc(-c3nc(-c4ccc5c(ccc6ccc7ccc8nc(-c9ccccc9)oc8c7c65)c4)nc(-c4cccc5sc6ccccc6c45)n3)c2)cc1 ; c1ccc(-c2nc3ccc4ccc5ccc6cc(-c7nc(-c8ccccc8-c8ccccc8)nc(-c8cccc9c8oc8ccccc89)n7)ccc6c5c4c3o2)cc1]

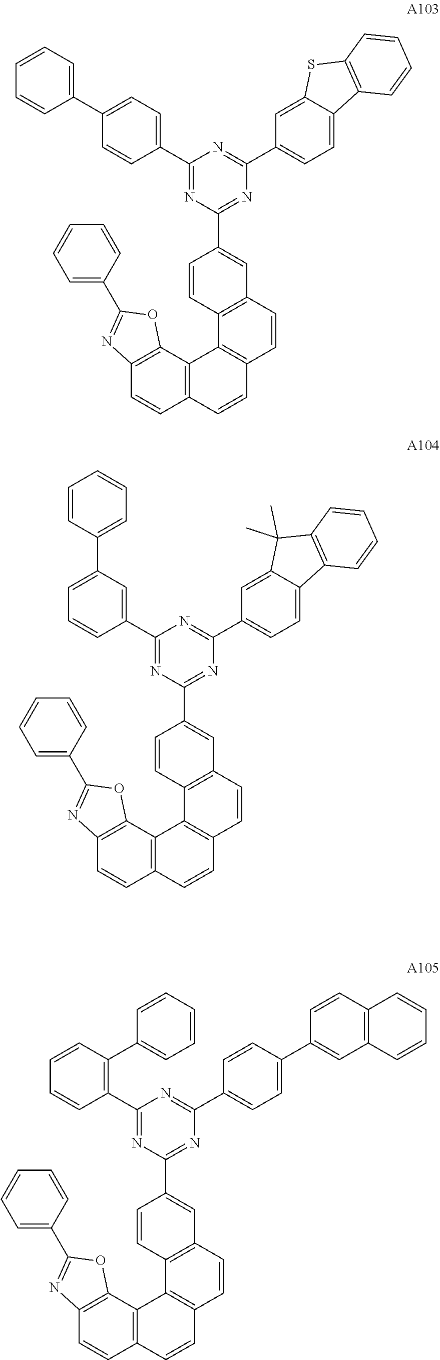 [SMILES: c1ccc(-c2ccc(-c3nc(-c4ccc5c(ccc6ccc7ccc8nc(-c9ccccc9)oc8c7c65)c4)nc(-c4ccc5c(c4)sc4ccccc45)n3)cc2)cc1 ; CC1(C)c2ccccc2-c2ccc(-c3nc(-c4cccc(-c5ccccc5)c4)nc(-c4ccc5c(ccc6ccc7ccc8nc(-c9ccccc9)oc8c7c65)c4)n3)cc21 ; c1ccc(-c2nc3ccc4ccc5ccc6cc(-c7nc(-c8ccc(-c9ccc%10ccccc%10c9)cc8)nc(-c8ccccc8-c8ccccc8)n7)ccc6c5c4c3o2)cc1]

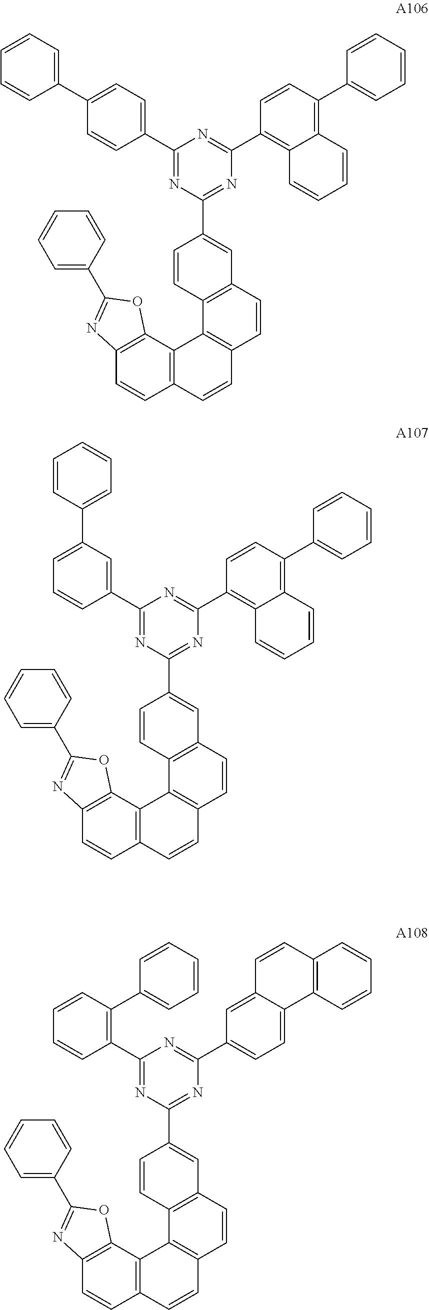 [SMILES: c1ccc(-c2ccc(-c3nc(-c4ccc5c(ccc6ccc7ccc8nc(-c9ccccc9)oc8c7c65)c4)nc(-c4ccc(-c5ccccc5)c5ccccc45)n3)cc2)cc1 ; c1ccc(-c2cccc(-c3nc(-c4ccc5c(ccc6ccc7ccc8nc(-c9ccccc9)oc8c7c65)c4)nc(-c4ccc(-c5ccccc5)c5ccccc45)n3)c2)cc1 ; c1ccc(-c2nc3ccc4ccc5ccc6cc(-c7nc(-c8ccc9c(ccc%10ccccc%109)c8)nc(-c8ccccc8-c8ccccc8)n7)ccc6c5c4c3o2)cc1]

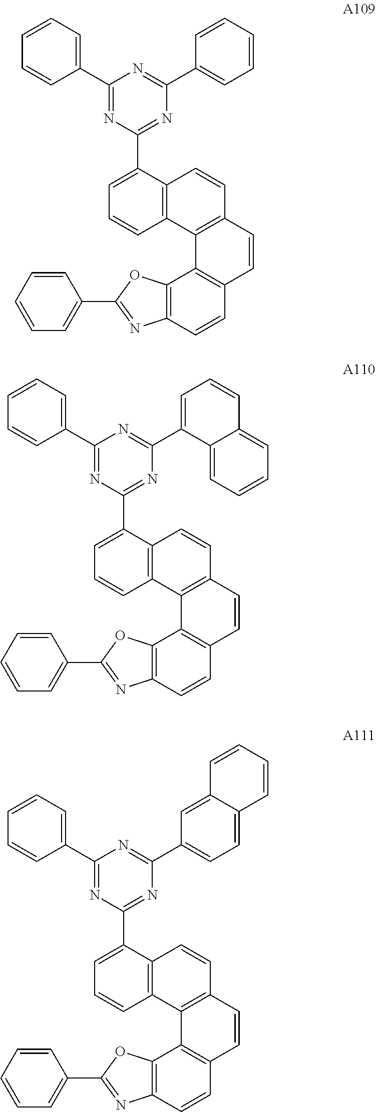

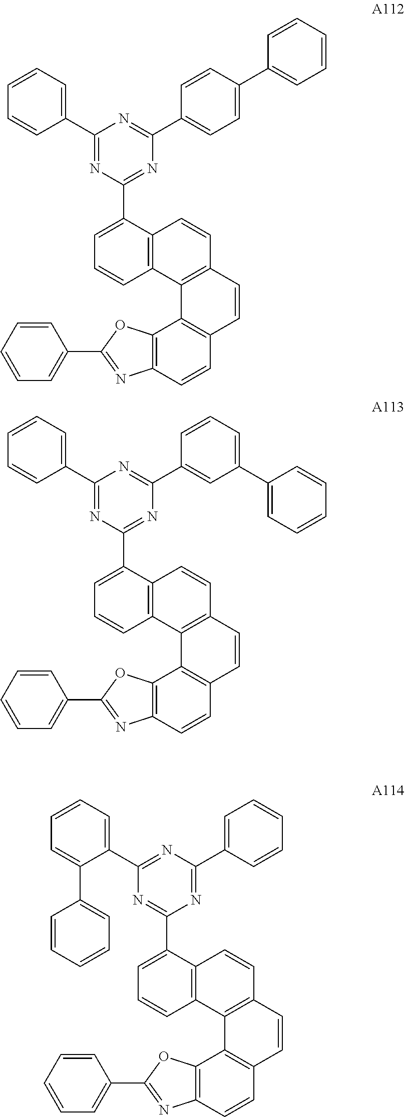

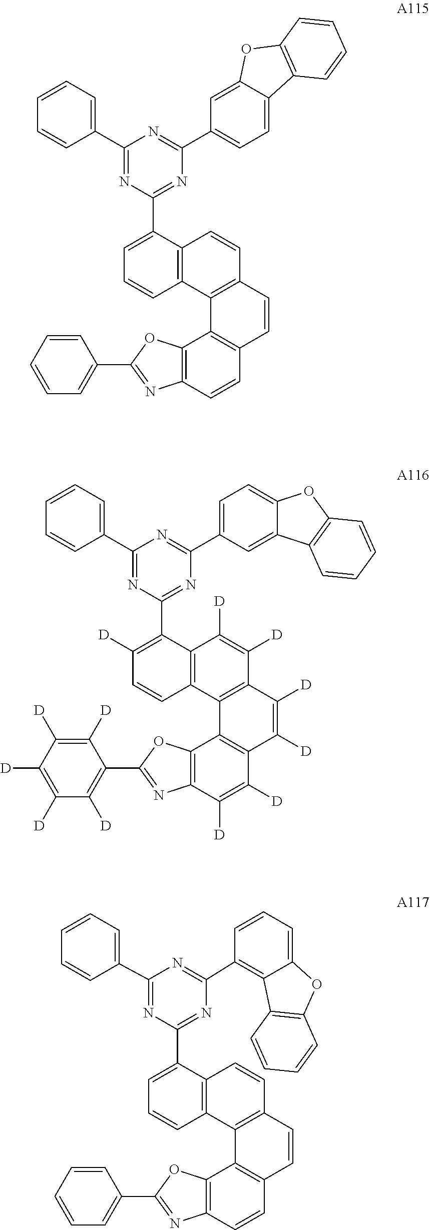 [SMILES: c1ccc(-c2nc(-c3ccc4c(c3)oc3ccccc34)nc(-c3cccc4c3ccc3ccc5ccc6nc(-c7ccccc7)oc6c5c34)n2)cc1 ; [2H]c1ccc2c(c([2H])c([2H])c3c([2H])c([2H])c4c([2H])c([2H])c5nc(-c6c([2H])c([2H])c([2H])c([2H])c6[2H])oc5c4c32)c1-c1nc(-c2ccccc2)nc(-c2ccc3oc4ccccc4c3c2)n1 ; c1ccc(-c2nc(-c3cccc4c3ccc3ccc5ccc6nc(-c7ccccc7)oc6c5c34)nc(-c3cccc4oc5ccccc5c34)n2)cc1]

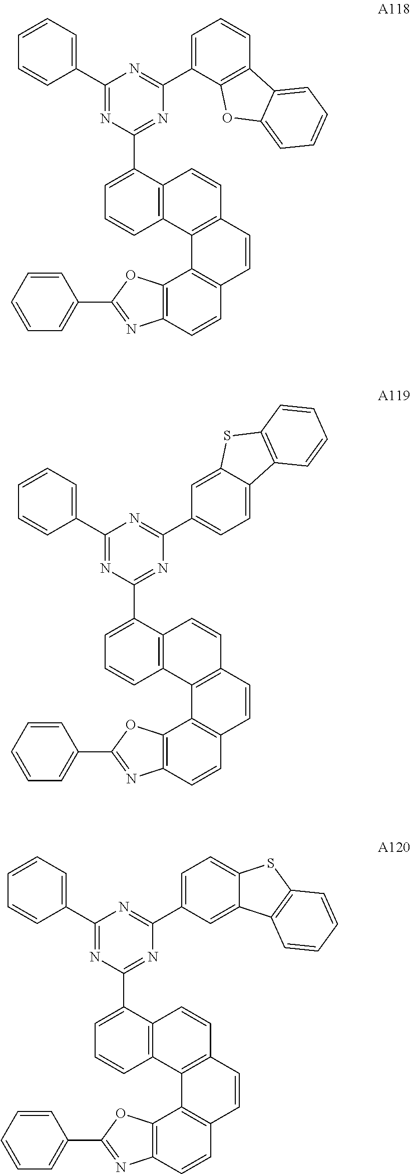 [SMILES: c1ccc(-c2nc(-c3cccc4c3ccc3ccc5ccc6nc(-c7ccccc7)oc6c5c34)nc(-c3cccc4c3oc3ccccc34)n2)cc1 ; c1ccc(-c2nc(-c3ccc4c(c3)sc3ccccc34)nc(-c3cccc4c3ccc3ccc5ccc6nc(-c7ccccc7)oc6c5c34)n2)cc1 ; c1ccc(-c2nc(-c3ccc4sc5ccccc5c4c3)nc(-c3cccc4c3ccc3ccc5ccc6nc(-c7ccccc7)oc6c5c34)n2)cc1]

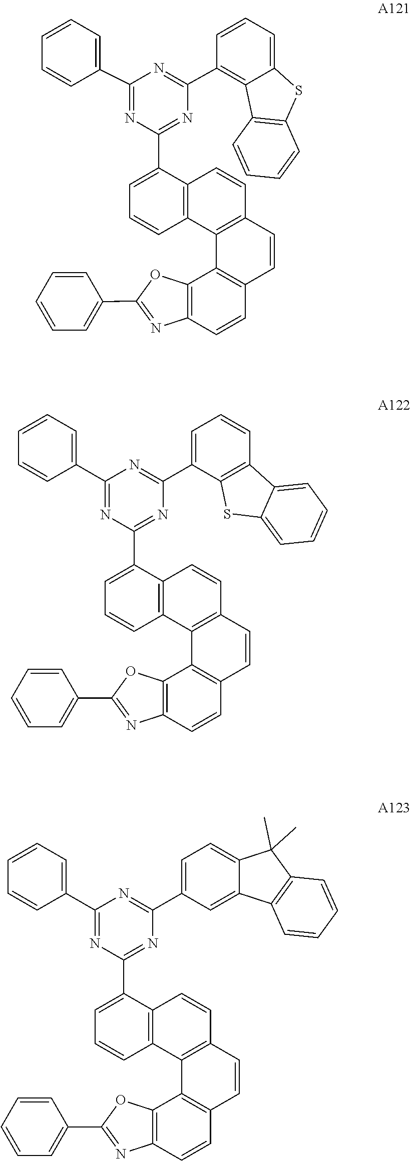 [SMILES: c1ccc(-c2nc(-c3cccc4c3ccc3ccc5ccc6nc(-c7ccccc7)oc6c5c34)nc(-c3cccc4sc5ccccc5c34)n2)cc1 ; c1ccc(-c2nc(-c3cccc4c3ccc3ccc5ccc6nc(-c7ccccc7)oc6c5c34)nc(-c3cccc4c3sc3ccccc34)n2)cc1 ; CC1(C)c2ccccc2-c2cc(-c3nc(-c4ccccc4)nc(-c4cccc5c4ccc4ccc6ccc7nc(-c8ccccc8)oc7c6c45)n3)ccc21]

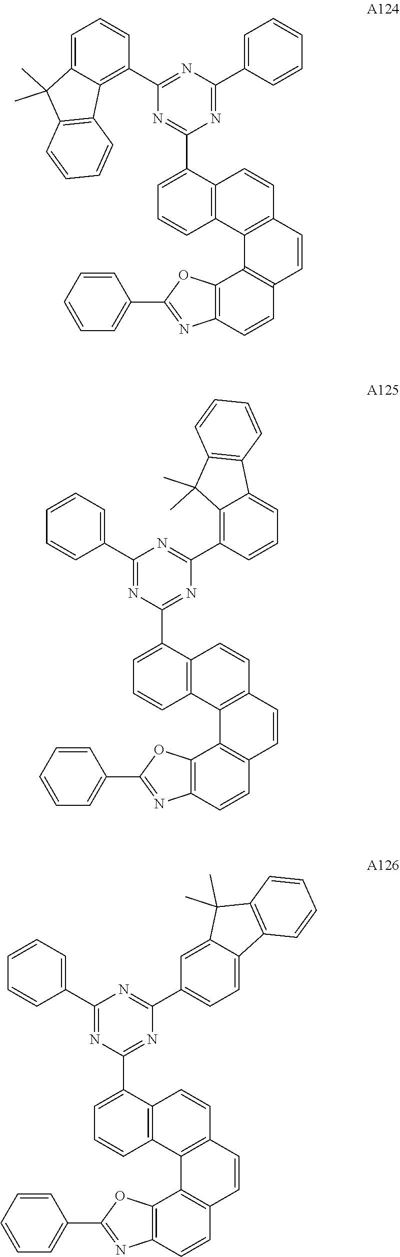 [SMILES: CC1(C)c2ccccc2-c2c(-c3nc(-c4ccccc4)nc(-c4cccc5c4ccc4ccc6ccc7nc(-c8ccccc8)oc7c6c45)n3)cccc21 ; CC1(C)c2ccccc2-c2cccc(-c3nc(-c4ccccc4)nc(-c4cccc5c4ccc4ccc6ccc7nc(-c8ccccc8)oc7c6c45)n3)c21 ; CC1(C)c2ccccc2-c2ccc(-c3nc(-c4ccccc4)nc(-c4cccc5c4ccc4ccc6ccc7nc(-c8ccccc8)oc7c6c45)n3)cc21]

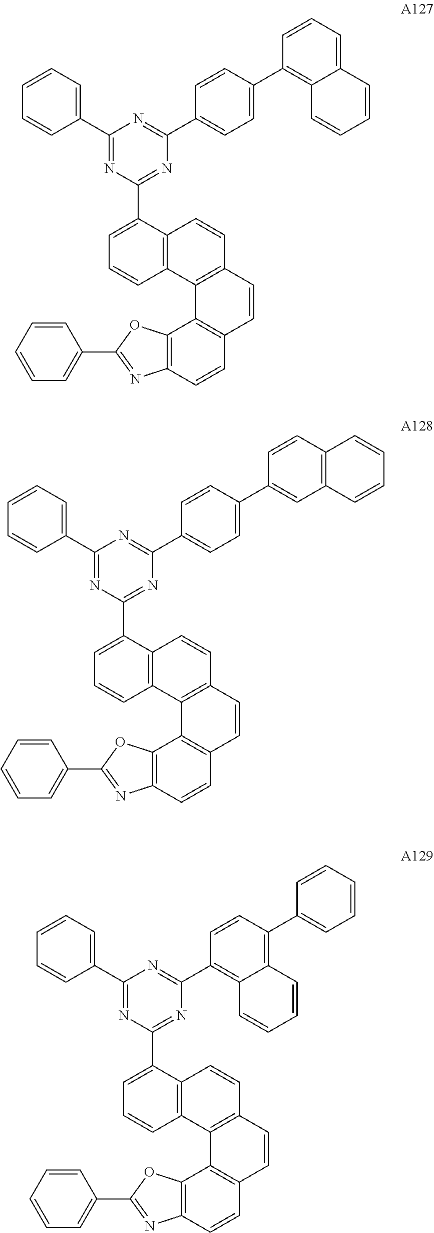 [SMILES: c1ccc(-c2nc(-c3ccc(-c4cccc5ccccc45)cc3)nc(-c3cccc4c3ccc3ccc5ccc6nc(-c7ccccc7)oc6c5c34)n2)cc1 ; c1ccc(-c2nc(-c3ccc(-c4ccc5ccccc5c4)cc3)nc(-c3cccc4c3ccc3ccc5ccc6nc(-c7ccccc7)oc6c5c34)n2)cc1 ; c1ccc(-c2nc(-c3ccc(-c4ccccc4)c4ccccc34)nc(-c3cccc4c3ccc3ccc5ccc6nc(-c7ccccc7)oc6c5c34)n2)cc1]

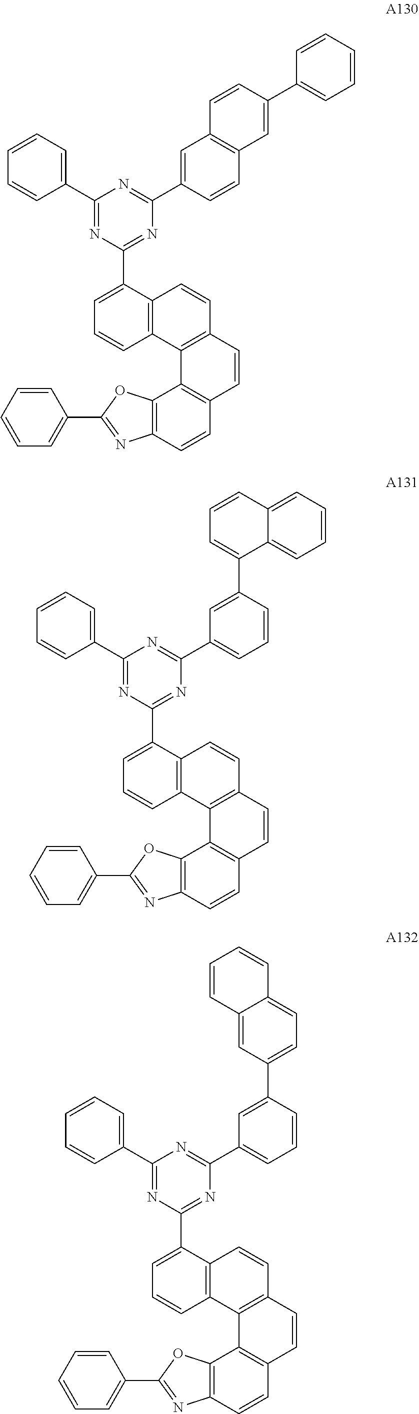 [SMILES: c1ccc(-c2ccc3cc(-c4nc(-c5ccccc5)nc(-c5cccc6c5ccc5ccc7ccc8nc(-c9ccccc9)oc8c7c56)n4)ccc3c2)cc1 ; c1ccc(-c2nc(-c3cccc(-c4cccc5ccccc45)c3)nc(-c3cccc4c3ccc3ccc5ccc6nc(-c7ccccc7)oc6c5c34)n2)cc1 ; c1ccc(-c2nc(-c3cccc(-c4ccc5ccccc5c4)c3)nc(-c3cccc4c3ccc3ccc5ccc6nc(-c7ccccc7)oc6c5c34)n2)cc1]

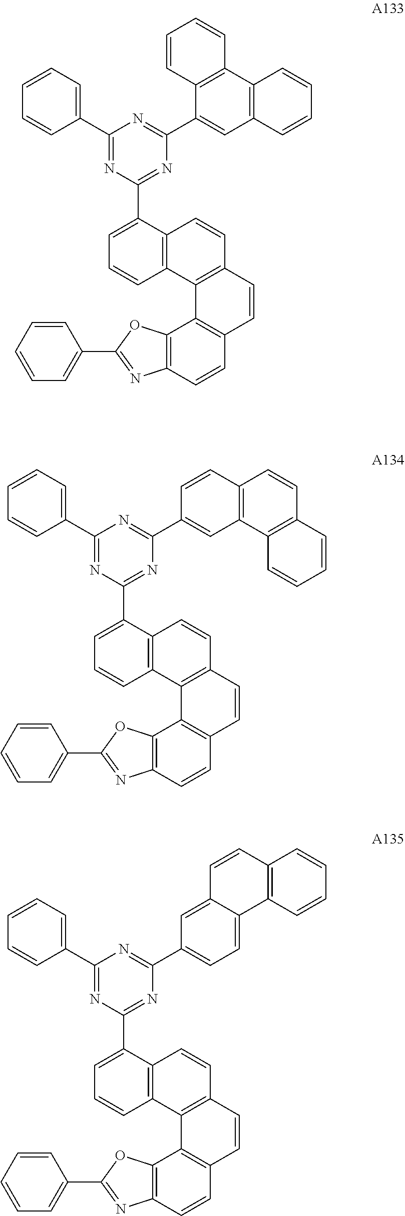 [SMILES: c1ccc(-c2nc(-c3cc4ccccc4c4ccccc34)nc(-c3cccc4c3ccc3ccc5ccc6nc(-c7ccccc7)oc6c5c34)n2)cc1 ; c1ccc(-c2nc(-c3ccc4ccc5ccccc5c4c3)nc(-c3cccc4c3ccc3ccc5ccc6nc(-c7ccccc7)oc6c5c34)n2)cc1 ; c1ccc(-c2nc(-c3ccc4c(ccc5ccccc54)c3)nc(-c3cccc4c3ccc3ccc5ccc6nc(-c7ccccc7)oc6c5c34)n2)cc1]

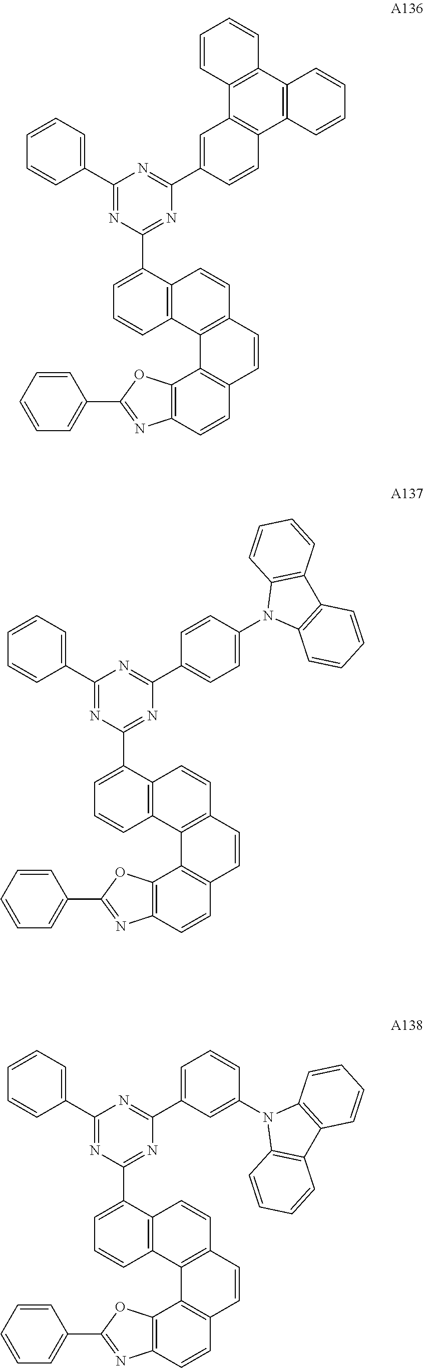 [SMILES: c1ccc(-c2nc(-c3ccc4c5ccccc5c5ccccc5c4c3)nc(-c3cccc4c3ccc3ccc5ccc6nc(-c7ccccc7)oc6c5c34)n2)cc1 ; c1ccc(-c2nc(-c3ccc(-n4c5ccccc5c5ccccc54)cc3)nc(-c3cccc4c3ccc3ccc5ccc6nc(-c7ccccc7)oc6c5c34)n2)cc1 ; c1ccc(-c2nc(-c3cccc(-n4c5ccccc5c5ccccc54)c3)nc(-c3cccc4c3ccc3ccc5ccc6nc(-c7ccccc7)oc6c5c34)n2)cc1]

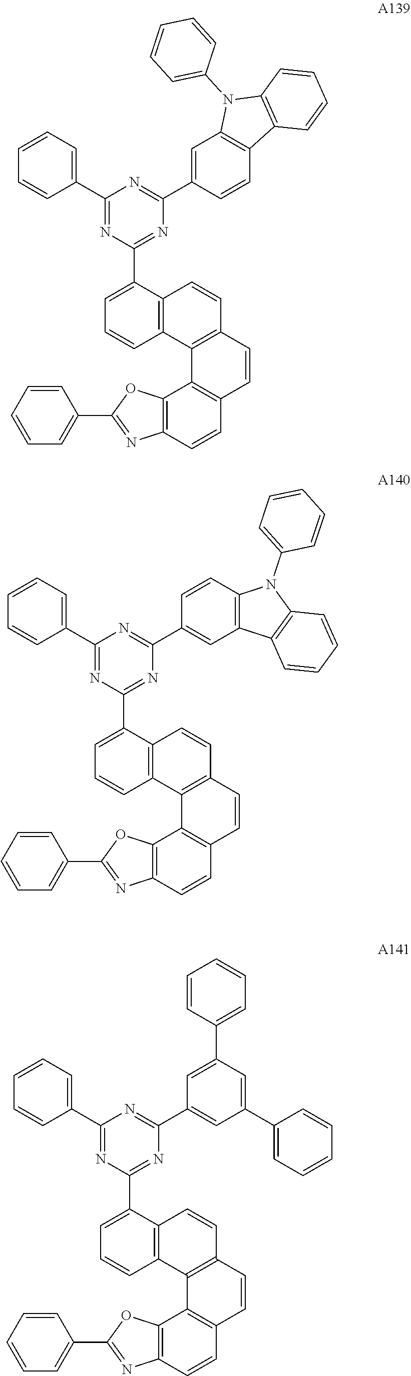 [SMILES: c1ccc(-c2nc(-c3ccc4c5ccccc5n(-c5ccccc5)c4c3)nc(-c3cccc4c3ccc3ccc5ccc6nc(-c7ccccc7)oc6c5c34)n2)cc1 ; c1ccc(-c2nc(-c3ccc4c(c3)c3ccccc3n4-c3ccccc3)nc(-c3cccc4c3ccc3ccc5ccc6nc(-c7ccccc7)oc6c5c34)n2)cc1 ; c1ccc(-c2cc(-c3ccccc3)cc(-c3nc(-c4ccccc4)nc(-c4cccc5c4ccc4ccc6ccc7nc(-c8ccccc8)oc7c6c45)n3)c2)cc1]

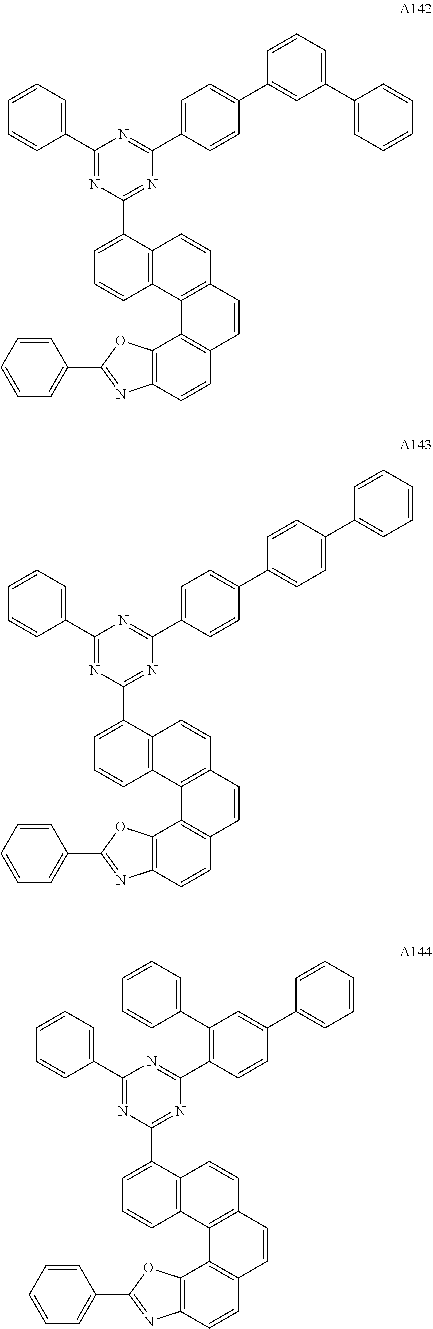 [SMILES: c1ccc(-c2cccc(-c3ccc(-c4nc(-c5ccccc5)nc(-c5cccc6c5ccc5ccc7ccc8nc(-c9ccccc9)oc8c7c56)n4)cc3)c2)cc1 ; c1ccc(-c2ccc(-c3ccc(-c4nc(-c5ccccc5)nc(-c5cccc6c5ccc5ccc7ccc8nc(-c9ccccc9)oc8c7c56)n4)cc3)cc2)cc1 ; c1ccc(-c2ccc(-c3nc(-c4ccccc4)nc(-c4cccc5c4ccc4ccc6ccc7nc(-c8ccccc8)oc7c6c45)n3)c(-c3ccccc3)c2)cc1]

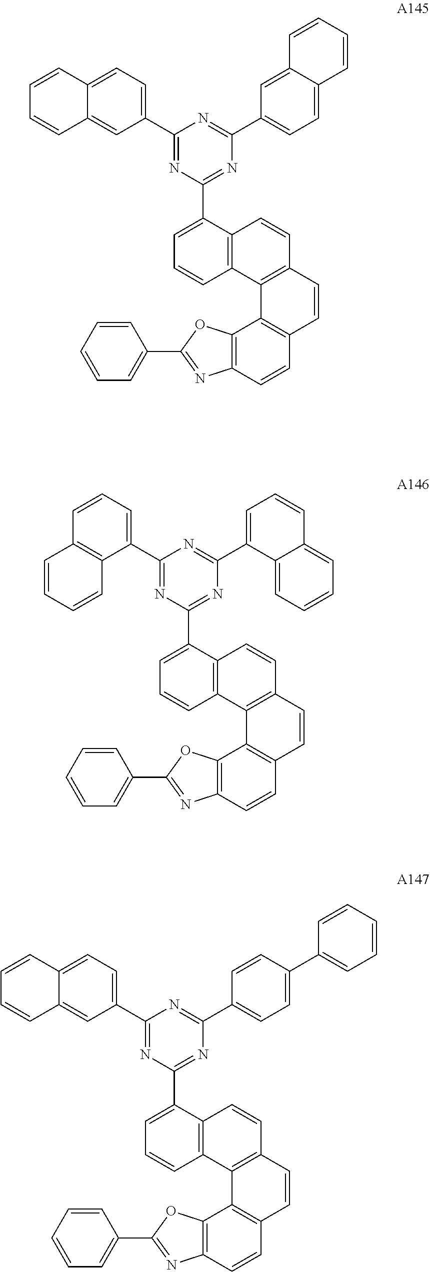 [SMILES: c1ccc(-c2nc3ccc4ccc5ccc6c(-c7nc(-c8ccc9ccccc9c8)nc(-c8ccc9ccccc9c8)n7)cccc6c5c4c3o2)cc1 ; c1ccc(-c2nc3ccc4ccc5ccc6c(-c7nc(-c8cccc9ccccc89)nc(-c8cccc9ccccc89)n7)cccc6c5c4c3o2)cc1 ; c1ccc(-c2ccc(-c3nc(-c4ccc5ccccc5c4)nc(-c4cccc5c4ccc4ccc6ccc7nc(-c8ccccc8)oc7c6c45)n3)cc2)cc1]

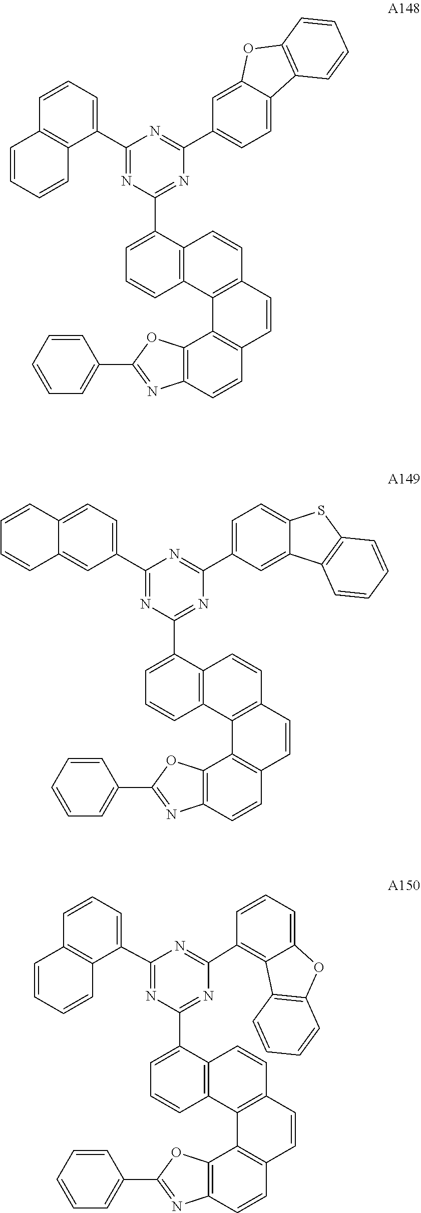 [SMILES: c1ccc(-c2nc3ccc4ccc5ccc6c(-c7nc(-c8ccc9c(c8)oc8ccccc89)nc(-c8cccc9ccccc89)n7)cccc6c5c4c3o2)cc1 ; c1ccc(-c2nc3ccc4ccc5ccc6c(-c7nc(-c8ccc9ccccc9c8)nc(-c8ccc9sc%10ccccc%10c9c8)n7)cccc6c5c4c3o2)cc1 ; c1ccc(-c2nc3ccc4ccc5ccc6c(-c7nc(-c8cccc9ccccc89)nc(-c8cccc9oc%10ccccc%10c89)n7)cccc6c5c4c3o2)cc1]

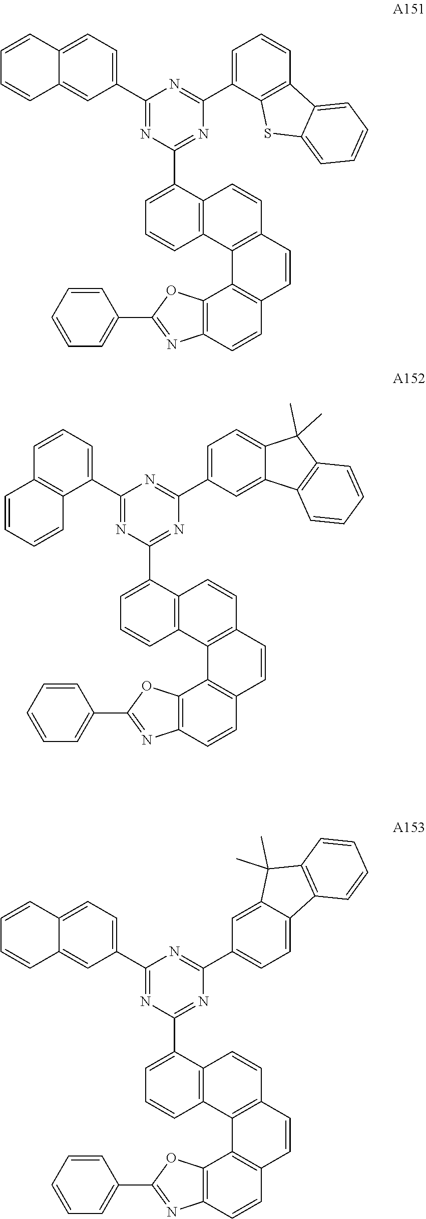 [SMILES: c1ccc(-c2nc3ccc4ccc5ccc6c(-c7nc(-c8ccc9ccccc9c8)nc(-c8cccc9c8sc8ccccc89)n7)cccc6c5c4c3o2)cc1 ; CC1(C)c2ccccc2-c2cc(-c3nc(-c4cccc5ccccc45)nc(-c4cccc5c4ccc4ccc6ccc7nc(-c8ccccc8)oc7c6c45)n3)ccc21 ; CC1(C)c2ccccc2-c2ccc(-c3nc(-c4ccc5ccccc5c4)nc(-c4cccc5c4ccc4ccc6ccc7nc(-c8ccccc8)oc7c6c45)n3)cc21]

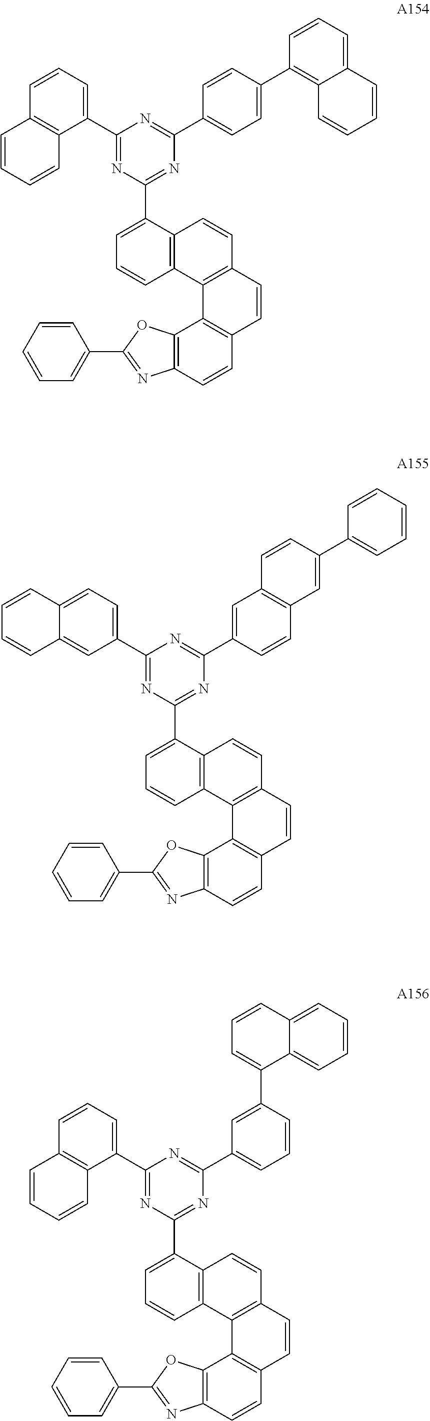 [SMILES: c1ccc(-c2nc3ccc4ccc5ccc6c(-c7nc(-c8ccc(-c9cccc%10ccccc9%10)cc8)nc(-c8cccc9ccccc89)n7)cccc6c5c4c3o2)cc1 ; c1ccc(-c2ccc3cc(-c4nc(-c5ccc6ccccc6c5)nc(-c5cccc6c5ccc5ccc7ccc8nc(-c9ccccc9)oc8c7c56)n4)ccc3c2)cc1 ; c1ccc(-c2nc3ccc4ccc5ccc6c(-c7nc(-c8cccc(-c9cccc%10ccccc9%10)c8)nc(-c8cccc9ccccc89)n7)cccc6c5c4c3o2)cc1]

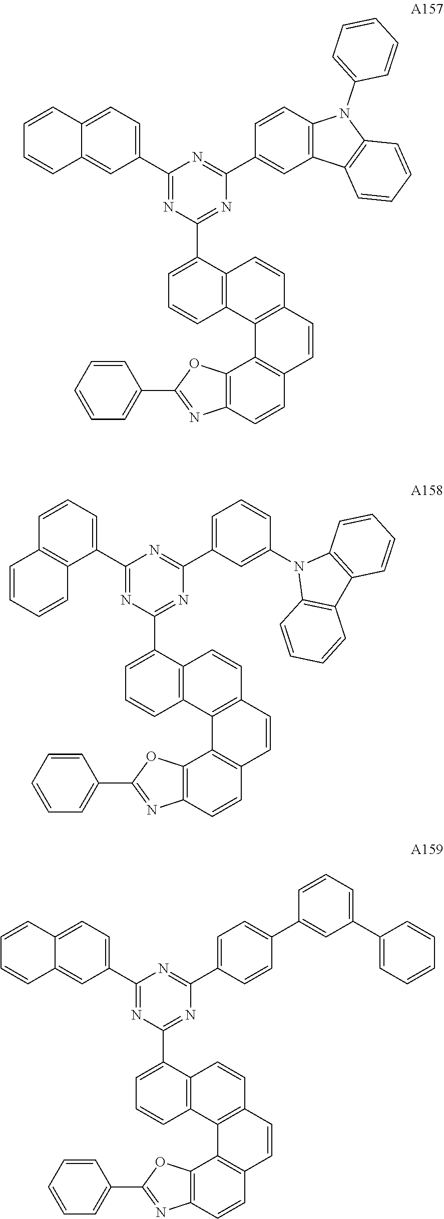 [SMILES: c1ccc(-c2nc3ccc4ccc5ccc6c(-c7nc(-c8ccc9ccccc9c8)nc(-c8ccc9c(c8)c8ccccc8n9-c8ccccc8)n7)cccc6c5c4c3o2)cc1 ; c1ccc(-c2nc3ccc4ccc5ccc6c(-c7nc(-c8cccc(-n9c%10ccccc%10c%10ccccc%109)c8)nc(-c8cccc9ccccc89)n7)cccc6c5c4c3o2)cc1 ; c1ccc(-c2cccc(-c3ccc(-c4nc(-c5ccc6ccccc6c5)nc(-c5cccc6c5ccc5ccc7ccc8nc(-c9ccccc9)oc8c7c56)n4)cc3)c2)cc1]

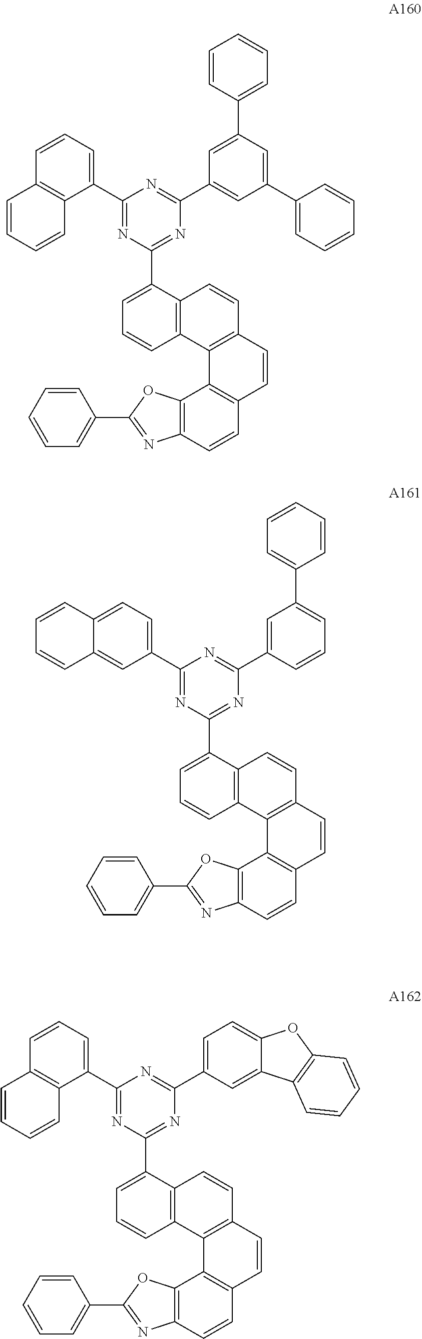 [SMILES: c1ccc(-c2cc(-c3ccccc3)cc(-c3nc(-c4cccc5ccccc45)nc(-c4cccc5c4ccc4ccc6ccc7nc(-c8ccccc8)oc7c6c45)n3)c2)cc1 ; c1ccc(-c2cccc(-c3nc(-c4ccc5ccccc5c4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8ccc9oc%10ccccc%10c9c8)nc(-c8cccc9ccccc89)n7)cccc6c5c4c3o2)cc1]

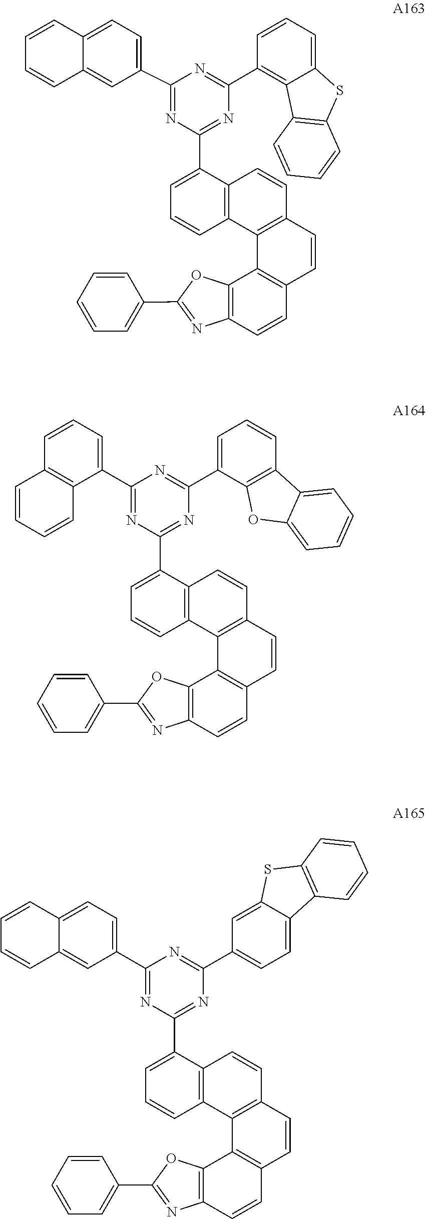 [SMILES: c1ccc(-c2nc3ccc4ccc5ccc6c(-c7nc(-c8ccc9ccccc9c8)nc(-c8cccc9sc%10ccccc%10c89)n7)cccc6c5c4c3o2)cc1 ; c1ccc(-c2nc3ccc4ccc5ccc6c(-c7nc(-c8cccc9ccccc89)nc(-c8cccc9c8oc8ccccc89)n7)cccc6c5c4c3o2)cc1 ; c1ccc(-c2nc3ccc4ccc5ccc6c(-c7nc(-c8ccc9ccccc9c8)nc(-c8ccc9c(c8)sc8ccccc89)n7)cccc6c5c4c3o2)cc1]

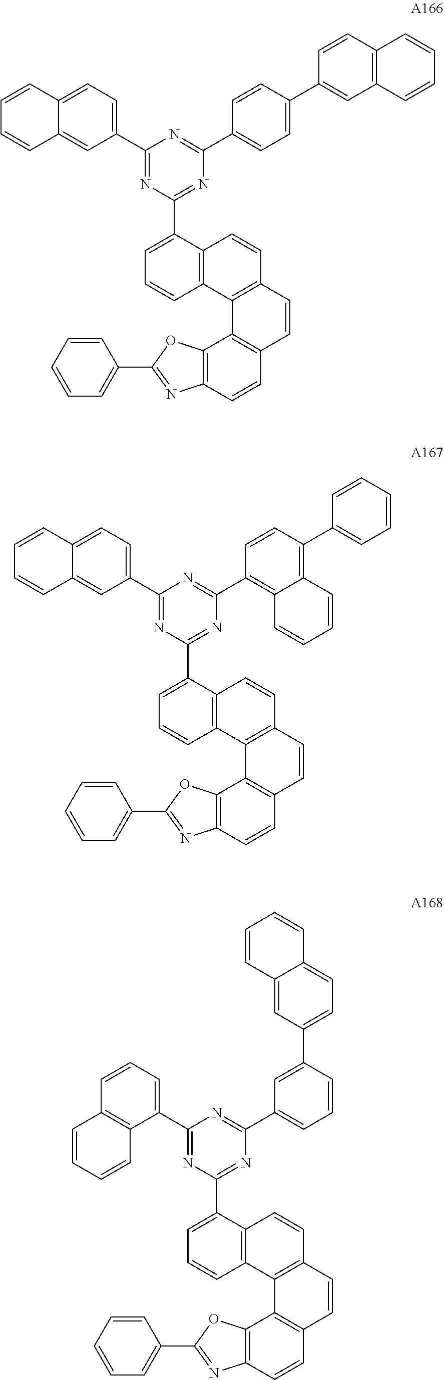 [SMILES: c1ccc(-c2nc3ccc4ccc5ccc6c(-c7nc(-c8ccc(-c9ccc%10ccccc%10c9)cc8)nc(-c8ccc9ccccc9c8)n7)cccc6c5c4c3o2)cc1 ; c1ccc(-c2nc3ccc4ccc5ccc6c(-c7nc(-c8ccc9ccccc9c8)nc(-c8ccc(-c9ccccc9)c9ccccc89)n7)cccc6c5c4c3o2)cc1 ; c1ccc(-c2nc3ccc4ccc5ccc6c(-c7nc(-c8cccc(-c9ccc%10ccccc%10c9)c8)nc(-c8cccc9ccccc89)n7)cccc6c5c4c3o2)cc1]

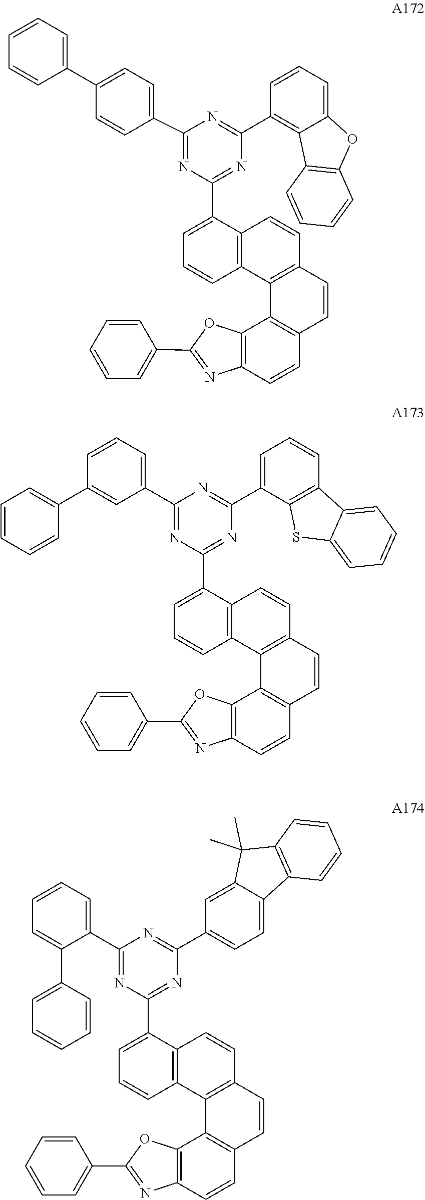 [SMILES: c1ccc(-c2ccc(-c3nc(-c4cccc5c4ccc4ccc6ccc7nc(-c8ccccc8)oc7c6c45)nc(-c4cccc5oc6ccccc6c45)n3)cc2)cc1 ; c1ccc(-c2cccc(-c3nc(-c4cccc5c4ccc4ccc6ccc7nc(-c8ccccc8)oc7c6c45)nc(-c4cccc5c4sc4ccccc45)n3)c2)cc1 ; CC1(C)c2ccccc2-c2ccc(-c3nc(-c4ccccc4-c4ccccc4)nc(-c4cccc5c4ccc4ccc6ccc7nc(-c8ccccc8)oc7c6c45)n3)cc21]

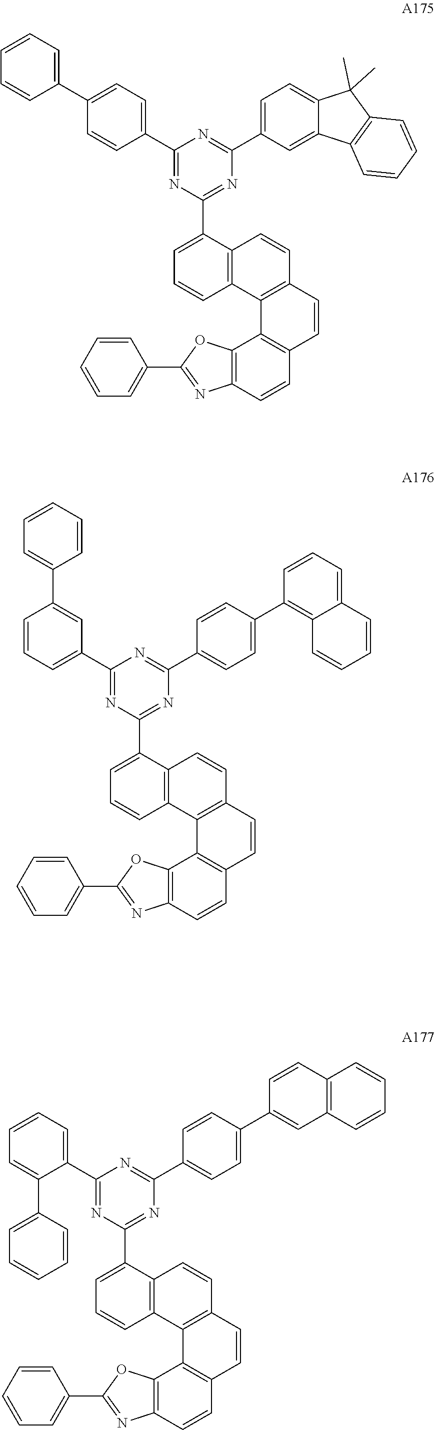 [SMILES: CC1(C)c2ccccc2-c2cc(-c3nc(-c4ccc(-c5ccccc5)cc4)nc(-c4cccc5c4ccc4ccc6ccc7nc(-c8ccccc8)oc7c6c45)n3)ccc21 ; c1ccc(-c2cccc(-c3nc(-c4ccc(-c5cccc6ccccc56)cc4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8ccc(-c9ccc%10ccccc%10c9)cc8)nc(-c8ccccc8-c8ccccc8)n7)cccc6c5c4c3o2)cc1]

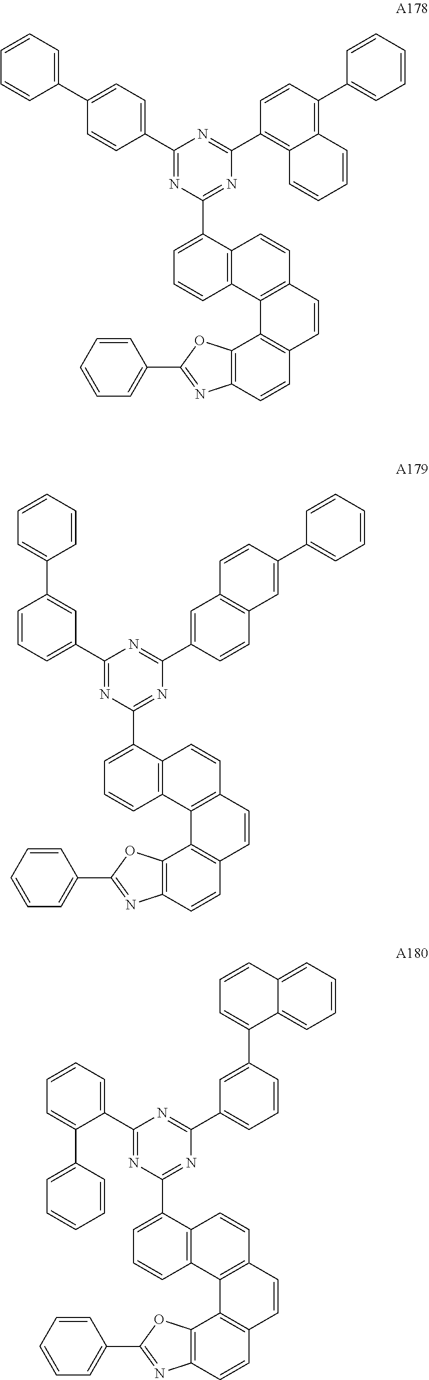 [SMILES: c1ccc(-c2ccc(-c3nc(-c4ccc(-c5ccccc5)c5ccccc45)nc(-c4cccc5c4ccc4ccc6ccc7nc(-c8ccccc8)oc7c6c45)n3)cc2)cc1 ; c1ccc(-c2cccc(-c3nc(-c4ccc5cc(-c6ccccc6)ccc5c4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8cccc(-c9cccc%10ccccc9%10)c8)nc(-c8ccccc8-c8ccccc8)n7)cccc6c5c4c3o2)cc1]

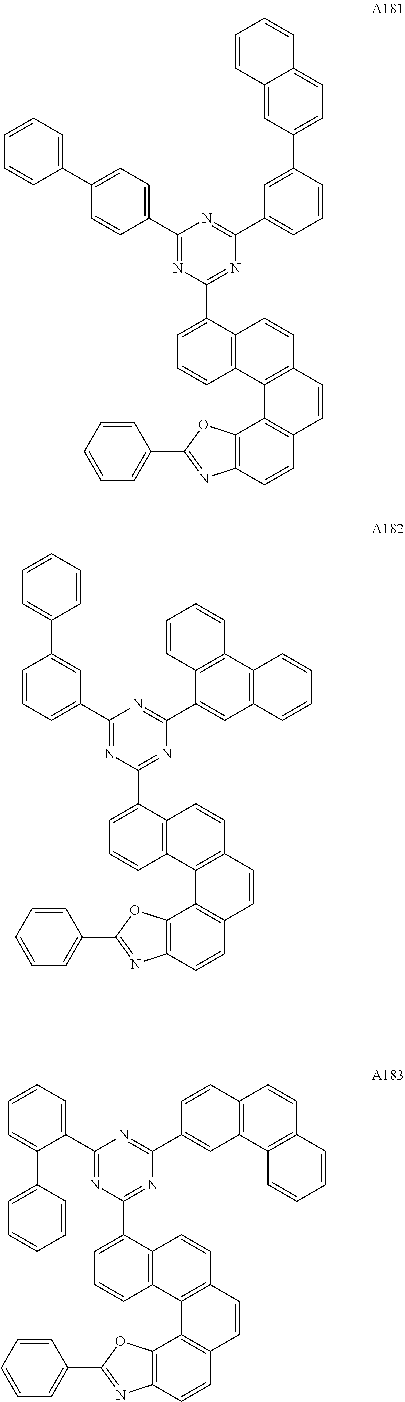 [SMILES: c1ccc(-c2ccc(-c3nc(-c4cccc(-c5ccc6ccccc6c5)c4)nc(-c4cccc5c4ccc4ccc6ccc7nc(-c8ccccc8)oc7c6c45)n3)cc2)cc1 ; c1ccc(-c2cccc(-c3nc(-c4cc5ccccc5c5ccccc45)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8ccc9ccc%10ccccc%10c9c8)nc(-c8ccccc8-c8ccccc8)n7)cccc6c5c4c3o2)cc1]

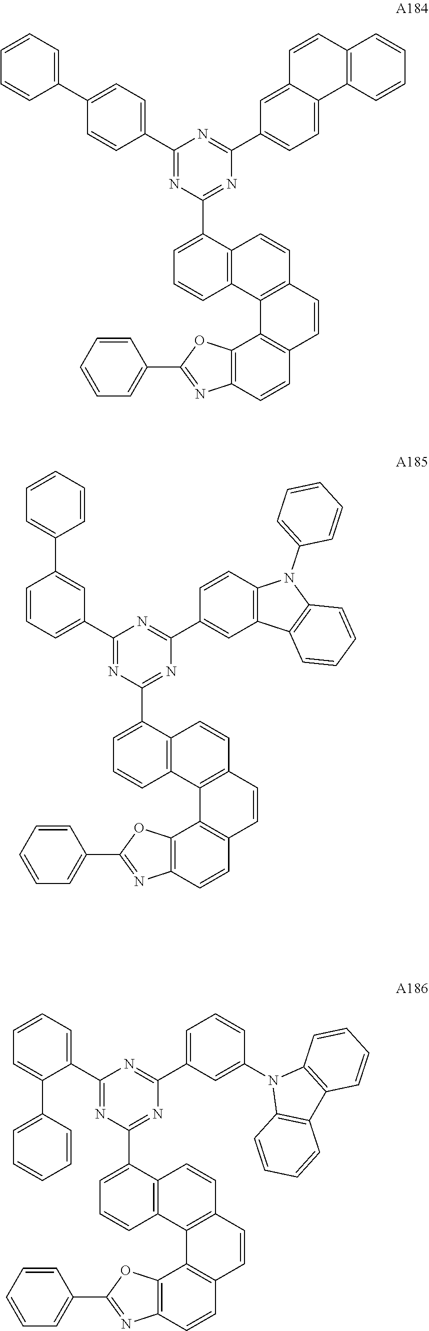 [SMILES: c1ccc(-c2ccc(-c3nc(-c4ccc5c(ccc6ccccc65)c4)nc(-c4cccc5c4ccc4ccc6ccc7nc(-c8ccccc8)oc7c6c45)n3)cc2)cc1 ; c1ccc(-c2cccc(-c3nc(-c4ccc5c(c4)c4ccccc4n5-c4ccccc4)nc(-c4cccc5c4ccc4ccc6ccc7nc(-c8ccccc8)oc7c6c45)n3)c2)cc1 ; c1ccc(-c2nc3ccc4ccc5ccc6c(-c7nc(-c8cccc(-n9c%10ccccc%10c%10ccccc%109)c8)nc(-c8ccccc8-c8ccccc8)n7)cccc6c5c4c3o2)cc1]

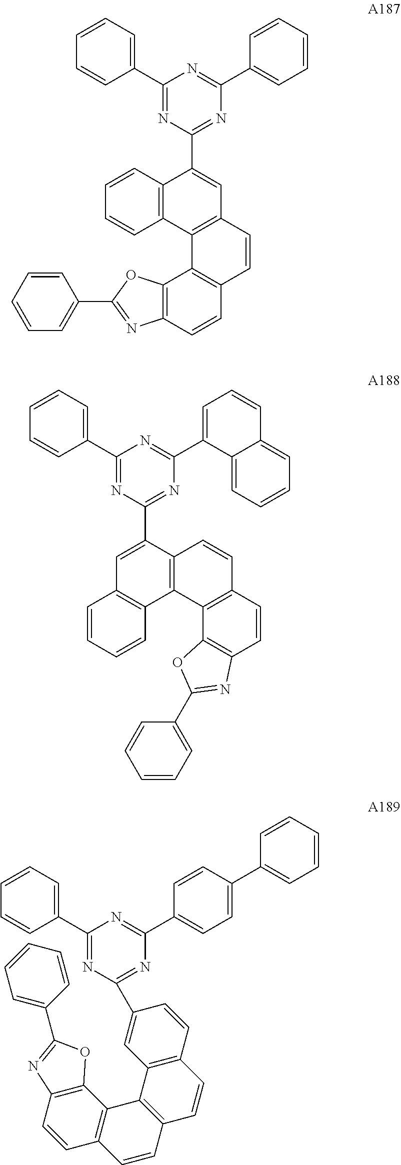 [SMILES: c1ccc(-c2nc(-c3ccccc3)nc(-c3cc4ccc5ccc6nc(-c7ccccc7)oc6c5c4c4ccccc34)n2)cc1 ; c1ccc(-c2nc(-c3cccc4ccccc34)nc(-c3cc4ccccc4c4c3ccc3ccc5nc(-c6ccccc6)oc5c34)n2)cc1 ; c1ccc(-c2ccc(-c3nc(-c4ccccc4)nc(-c4ccc5ccc6ccc7ccc8nc(-c9ccccc9)oc8c7c6c5c4)n3)cc2)cc1]

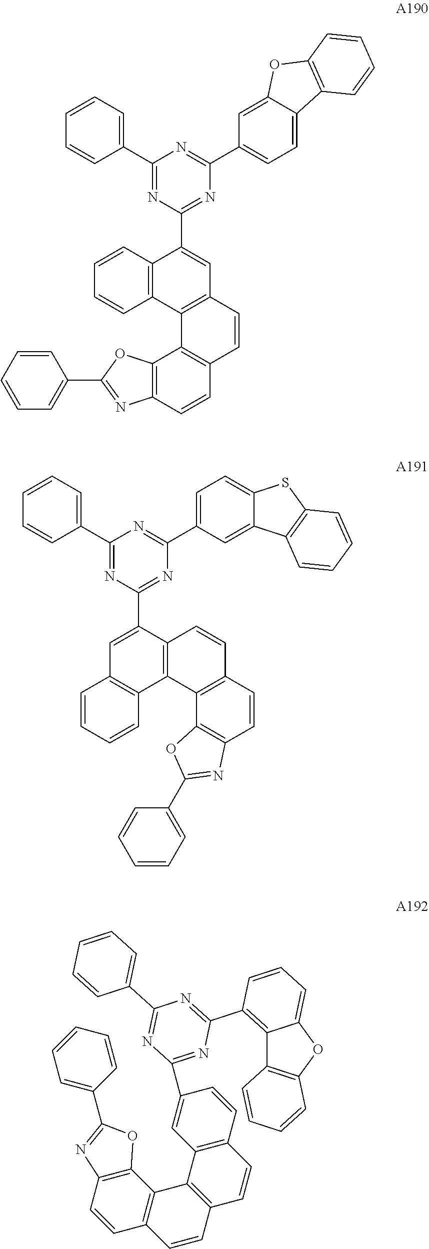 [SMILES: c1ccc(-c2nc(-c3ccc4c(c3)oc3ccccc34)nc(-c3cc4ccc5ccc6nc(-c7ccccc7)oc6c5c4c4ccccc34)n2)cc1 ; c1ccc(-c2nc(-c3ccc4sc5ccccc5c4c3)nc(-c3cc4ccccc4c4c3ccc3ccc5nc(-c6ccccc6)oc5c34)n2)cc1 ; c1ccc(-c2nc(-c3ccc4ccc5ccc6ccc7nc(-c8ccccc8)oc7c6c5c4c3)nc(-c3cccc4oc5ccccc5c34)n2)cc1]

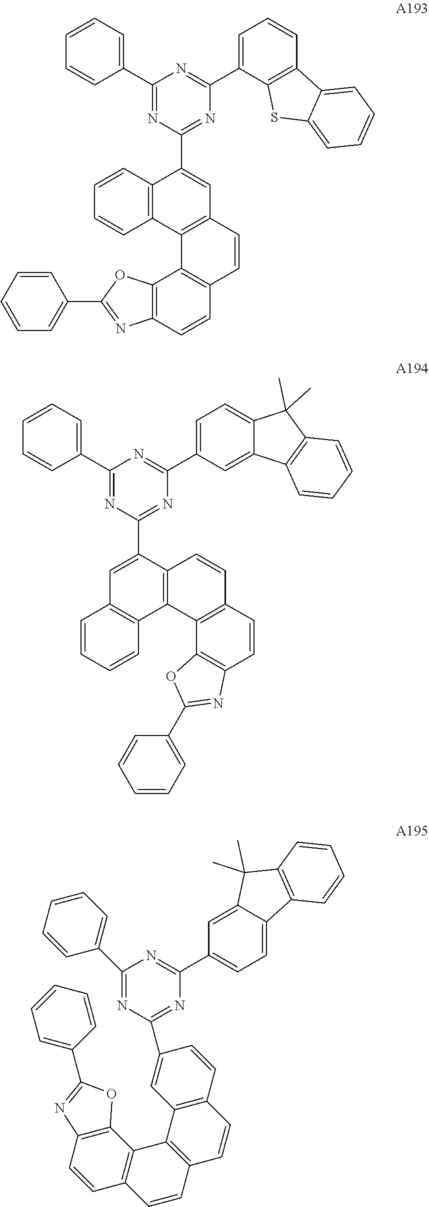 [SMILES: c1ccc(-c2nc(-c3cc4ccc5ccc6nc(-c7ccccc7)oc6c5c4c4ccccc34)nc(-c3cccc4c3sc3ccccc34)n2)cc1 ; CC1(C)c2ccccc2-c2cc(-c3nc(-c4ccccc4)nc(-c4cc5ccccc5c5c4ccc4ccc6nc(-c7ccccc7)oc6c45)n3)ccc21 ; CC1(C)c2ccccc2-c2ccc(-c3nc(-c4ccccc4)nc(-c4ccc5ccc6ccc7ccc8nc(-c9ccccc9)oc8c7c6c5c4)n3)cc21]

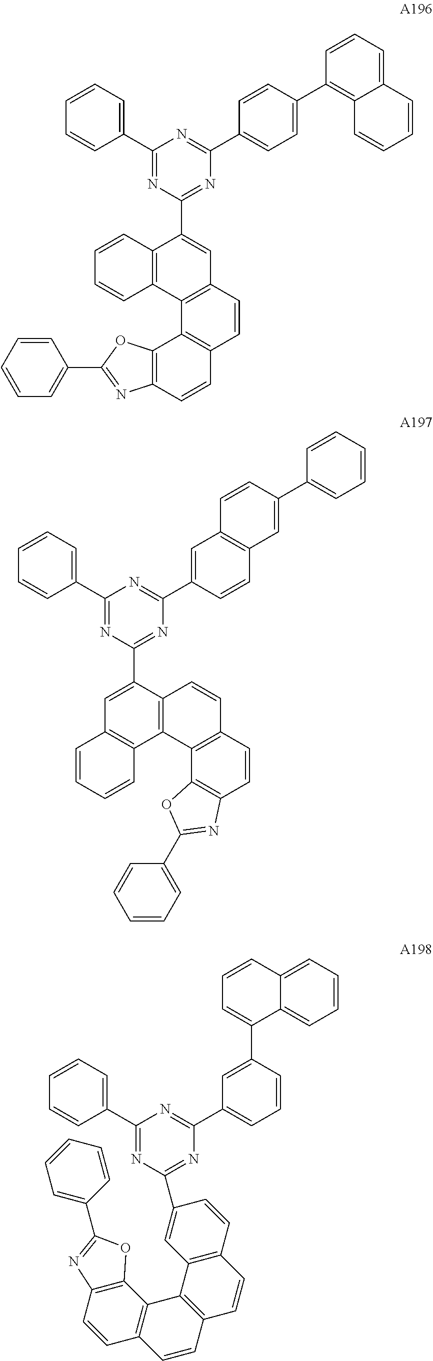 [SMILES: c1ccc(-c2nc(-c3ccc(-c4cccc5ccccc45)cc3)nc(-c3cc4ccc5ccc6nc(-c7ccccc7)oc6c5c4c4ccccc34)n2)cc1 ; c1ccc(-c2ccc3cc(-c4nc(-c5ccccc5)nc(-c5cc6ccccc6c6c5ccc5ccc7nc(-c8ccccc8)oc7c56)n4)ccc3c2)cc1 ; c1ccc(-c2nc(-c3cccc(-c4cccc5ccccc45)c3)nc(-c3ccc4ccc5ccc6ccc7nc(-c8ccccc8)oc7c6c5c4c3)n2)cc1]

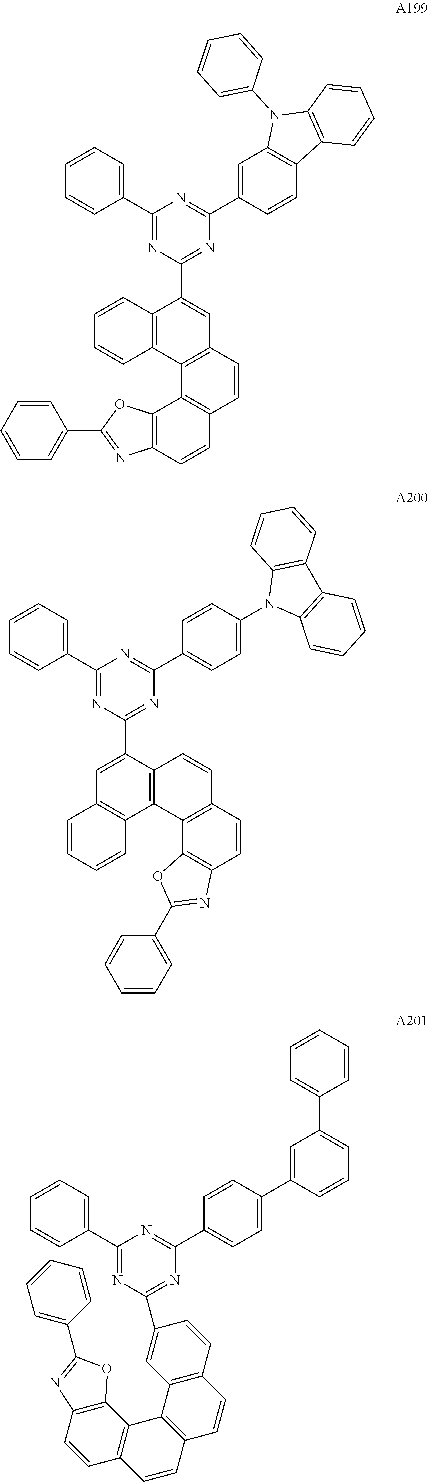 [SMILES: c1ccc(-c2nc(-c3ccc4c5ccccc5n(-c5ccccc5)c4c3)nc(-c3cc4ccc5ccc6nc(-c7ccccc7)oc6c5c4c4ccccc34)n2)cc1 ; c1ccc(-c2nc(-c3ccc(-n4c5ccccc5c5ccccc54)cc3)nc(-c3cc4ccccc4c4c3ccc3ccc5nc(-c6ccccc6)oc5c34)n2)cc1 ; c1ccc(-c2cccc(-c3ccc(-c4nc(-c5ccccc5)nc(-c5ccc6ccc7ccc8ccc9nc(-c%10ccccc%10)oc9c8c7c6c5)n4)cc3)c2)cc1]

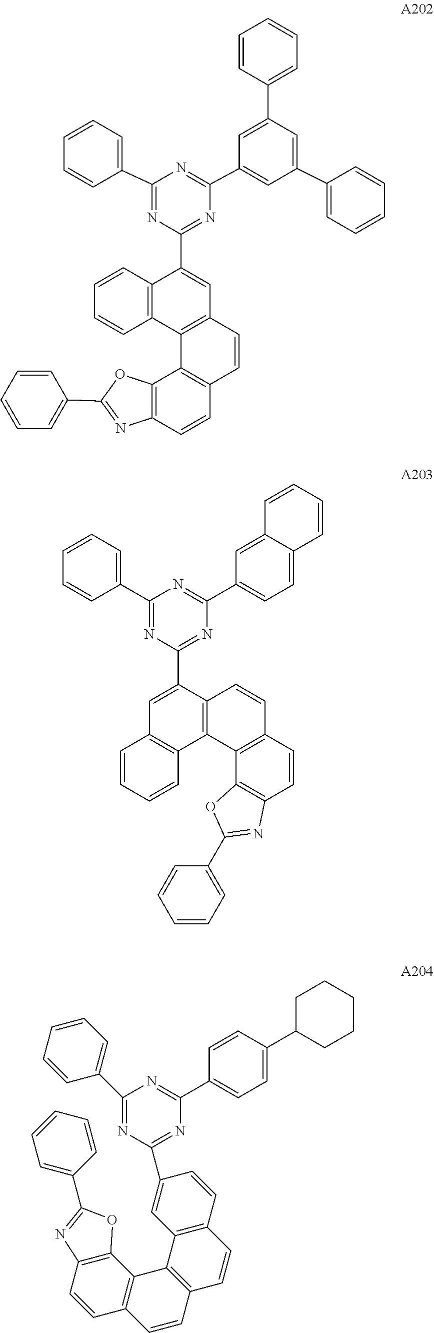 [SMILES: c1ccc(-c2cc(-c3ccccc3)cc(-c3nc(-c4ccccc4)nc(-c4cc5ccc6ccc7nc(-c8ccccc8)oc7c6c5c5ccccc45)n3)c2)cc1 ; c1ccc(-c2nc(-c3ccc4ccccc4c3)nc(-c3cc4ccccc4c4c3ccc3ccc5nc(-c6ccccc6)oc5c34)n2)cc1 ; c1ccc(-c2nc(-c3ccc(C4CCCCC4)cc3)nc(-c3ccc4ccc5ccc6ccc7nc(-c8ccccc8)oc7c6c5c4c3)n2)cc1]

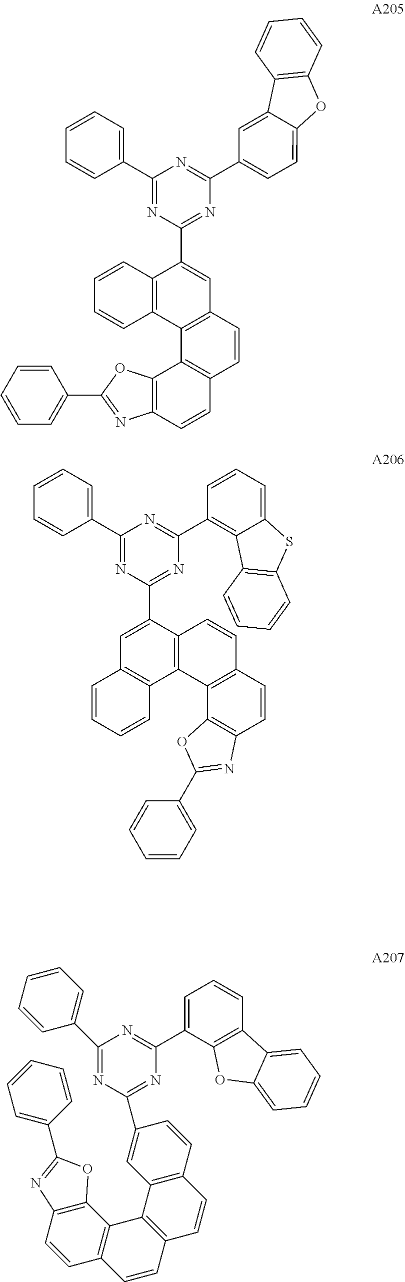

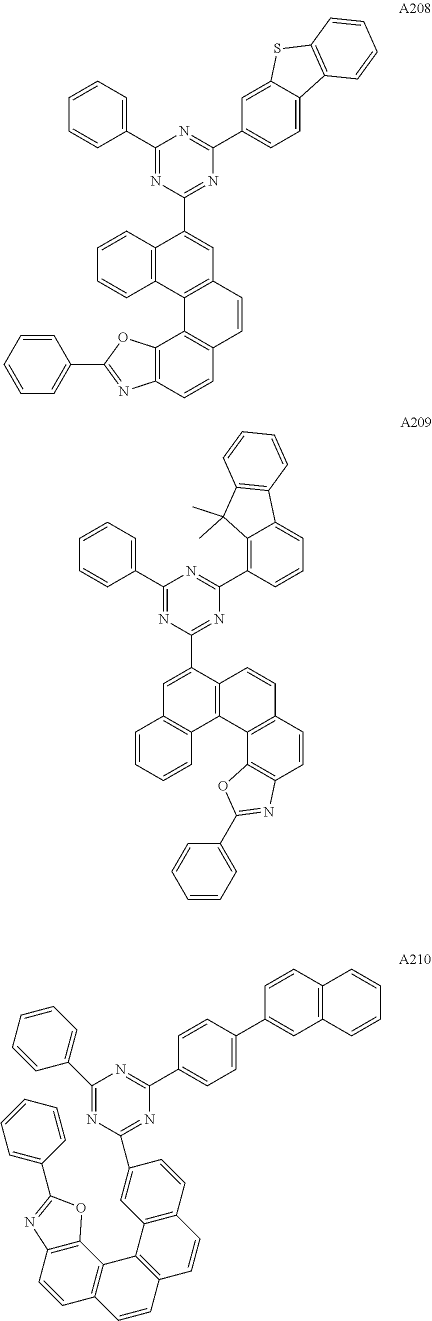 [SMILES: c1ccc(-c2nc(-c3ccc4c(c3)sc3ccccc34)nc(-c3cc4ccc5ccc6nc(-c7ccccc7)oc6c5c4c4ccccc34)n2)cc1 ; CC1(C)c2ccccc2-c2cccc(-c3nc(-c4ccccc4)nc(-c4cc5ccccc5c5c4ccc4ccc6nc(-c7ccccc7)oc6c45)n3)c21 ; c1ccc(-c2nc(-c3ccc(-c4ccc5ccccc5c4)cc3)nc(-c3ccc4ccc5ccc6ccc7nc(-c8ccccc8)oc7c6c5c4c3)n2)cc1]

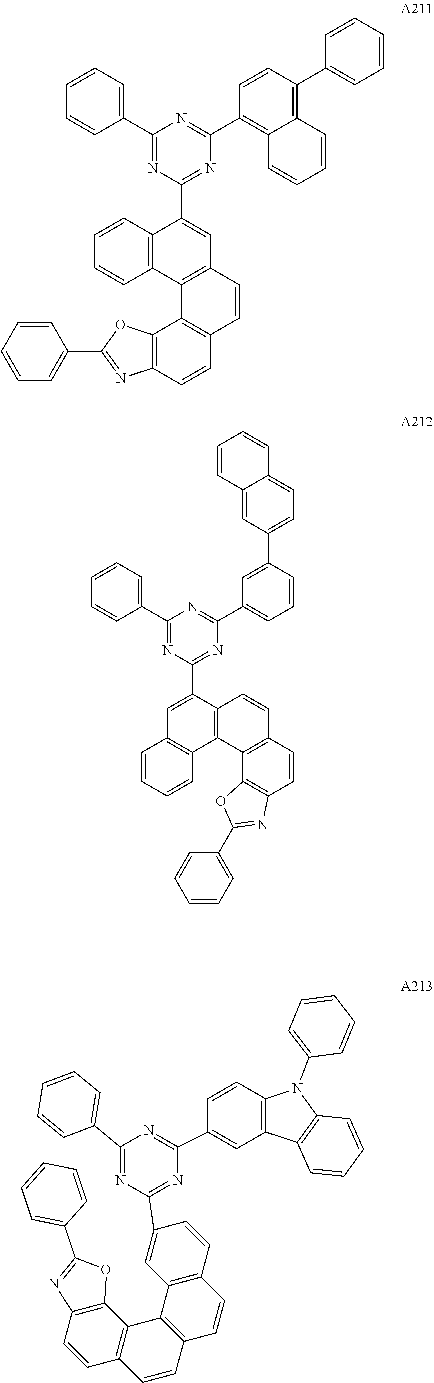 [SMILES: c1ccc(-c2nc(-c3ccc(-c4ccccc4)c4ccccc34)nc(-c3cc4ccc5ccc6nc(-c7ccccc7)oc6c5c4c4ccccc34)n2)cc1 ; c1ccc(-c2nc(-c3cccc(-c4ccc5ccccc5c4)c3)nc(-c3cc4ccccc4c4c3ccc3ccc5nc(-c6ccccc6)oc5c34)n2)cc1 ; c1ccc(-c2nc(-c3ccc4ccc5ccc6ccc7nc(-c8ccccc8)oc7c6c5c4c3)nc(-c3ccc4c(c3)c3ccccc3n4-c3ccccc3)n2)cc1]

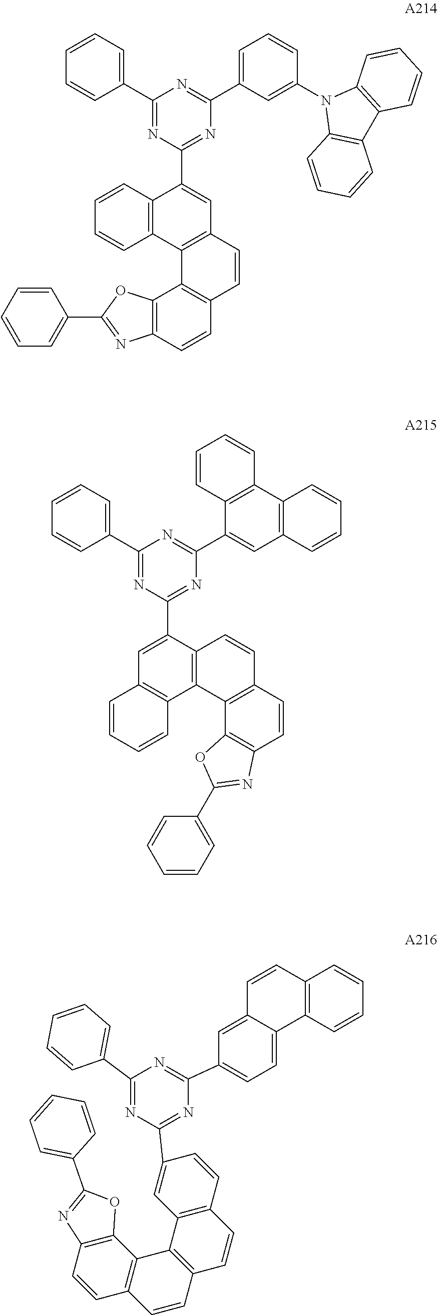 [SMILES: c1ccc(-c2nc(-c3cccc(-n4c5ccccc5c5ccccc54)c3)nc(-c3cc4ccc5ccc6nc(-c7ccccc7)oc6c5c4c4ccccc34)n2)cc1 ; c1ccc(-c2nc(-c3cc4ccccc4c4ccccc34)nc(-c3cc4ccccc4c4c3ccc3ccc5nc(-c6ccccc6)oc5c34)n2)cc1 ; c1ccc(-c2nc(-c3ccc4c(ccc5ccccc54)c3)nc(-c3ccc4ccc5ccc6ccc7nc(-c8ccccc8)oc7c6c5c4c3)n2)cc1]

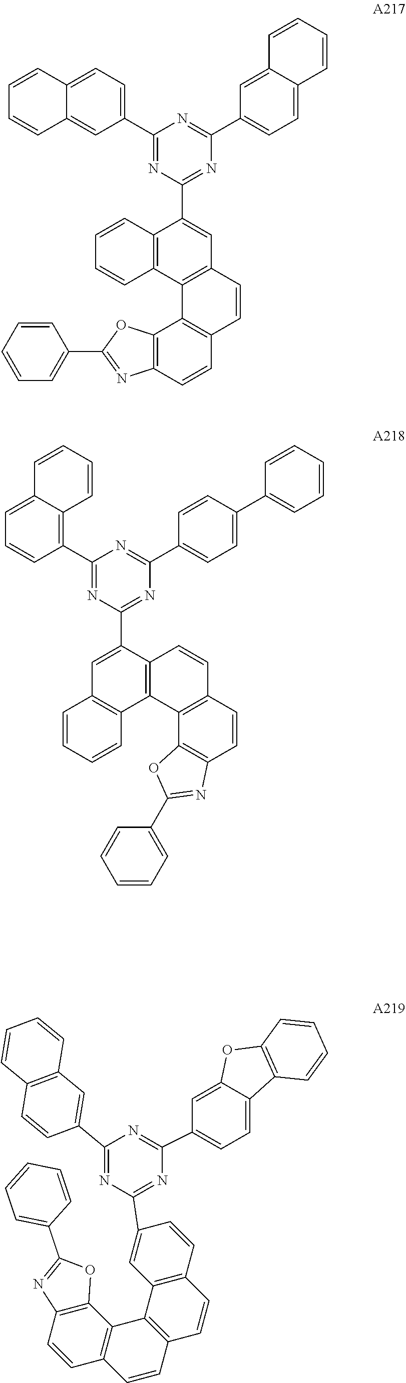 [SMILES: c1ccc(-c2nc3ccc4ccc5cc(-c6nc(-c7ccc8ccccc8c7)nc(-c7ccc8ccccc8c7)n6)c6ccccc6c5c4c3o2)cc1 ; c1ccc(-c2ccc(-c3nc(-c4cccc5ccccc45)nc(-c4cc5ccccc5c5c4ccc4ccc6nc(-c7ccccc7)oc6c45)n3)cc2)cc1 ; c1ccc(-c2nc3ccc4ccc5ccc6ccc(-c7nc(-c8ccc9ccccc9c8)nc(-c8ccc9c(c8)oc8ccccc89)n7)cc6c5c4c3o2)cc1]

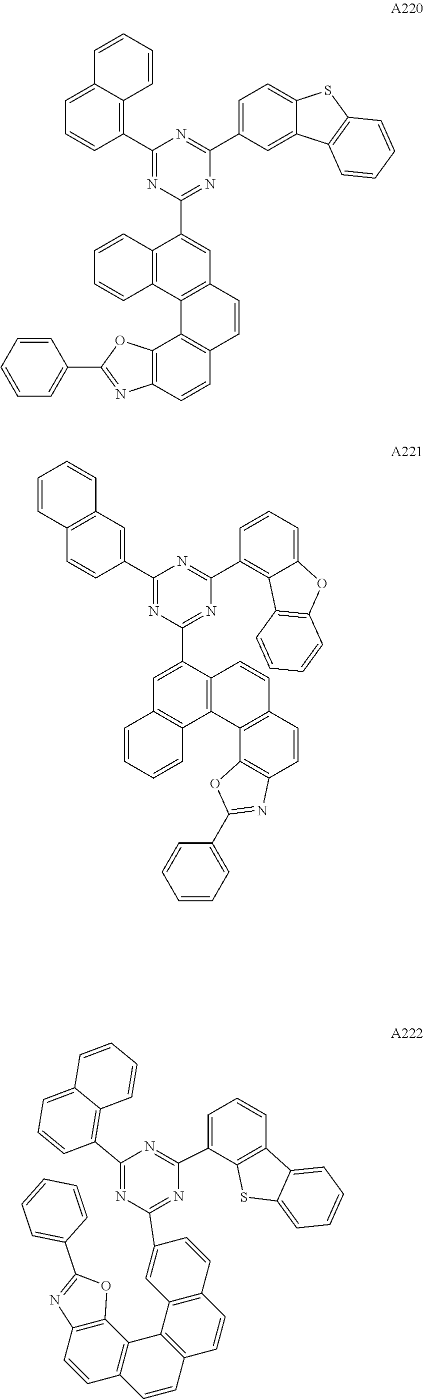 [SMILES: c1ccc(-c2nc3ccc4ccc5cc(-c6nc(-c7ccc8sc9ccccc9c8c7)nc(-c7cccc8ccccc78)n6)c6ccccc6c5c4c3o2)cc1 ; c1ccc(-c2nc3ccc4ccc5c(-c6nc(-c7ccc8ccccc8c7)nc(-c7cccc8oc9ccccc9c78)n6)cc6ccccc6c5c4c3o2)cc1 ; c1ccc(-c2nc3ccc4ccc5ccc6ccc(-c7nc(-c8cccc9ccccc89)nc(-c8cccc9c8sc8ccccc89)n7)cc6c5c4c3o2)cc1]

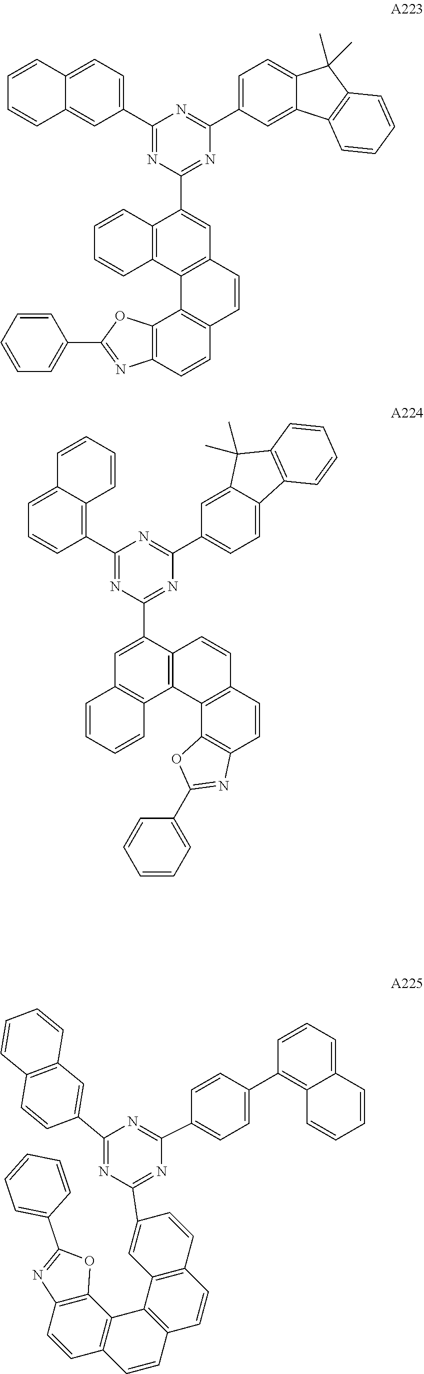 [SMILES: CC1(C)c2ccccc2-c2cc(-c3nc(-c4ccc5ccccc5c4)nc(-c4cc5ccc6ccc7nc(-c8ccccc8)oc7c6c5c5ccccc45)n3)ccc21 ; CC1(C)c2ccccc2-c2ccc(-c3nc(-c4cccc5ccccc45)nc(-c4cc5ccccc5c5c4ccc4ccc6nc(-c7ccccc7)oc6c45)n3)cc21 ; c1ccc(-c2nc3ccc4ccc5ccc6ccc(-c7nc(-c8ccc(-c9cccc%10ccccc9%10)cc8)nc(-c8ccc9ccccc9c8)n7)cc6c5c4c3o2)cc1]

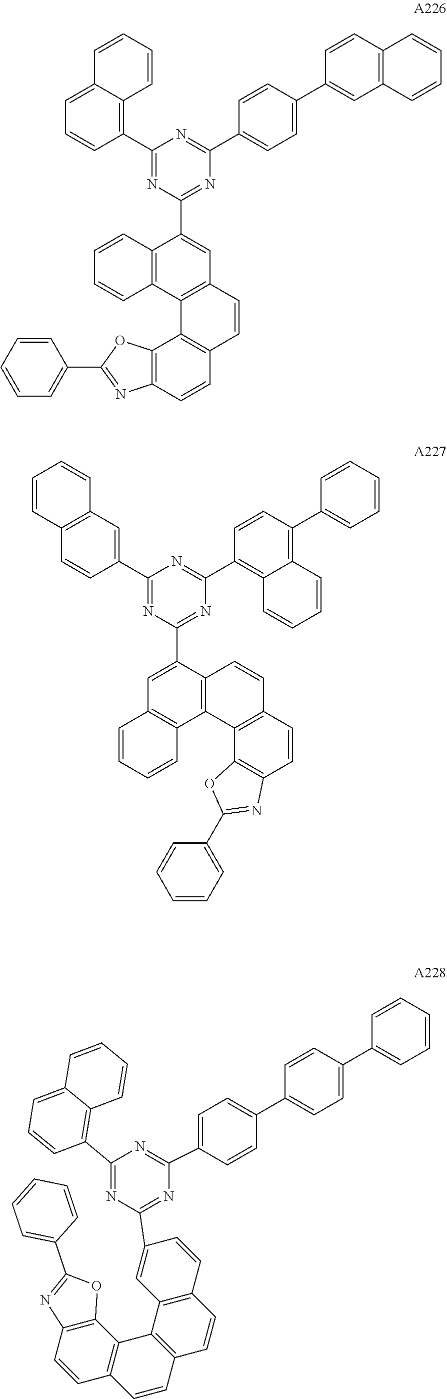 [SMILES: c1ccc(-c2nc3ccc4ccc5cc(-c6nc(-c7ccc(-c8ccc9ccccc9c8)cc7)nc(-c7cccc8ccccc78)n6)c6ccccc6c5c4c3o2)cc1 ; c1ccc(-c2nc3ccc4ccc5c(-c6nc(-c7ccc8ccccc8c7)nc(-c7ccc(-c8ccccc8)c8ccccc78)n6)cc6ccccc6c5c4c3o2)cc1 ; c1ccc(-c2ccc(-c3ccc(-c4nc(-c5ccc6ccc7ccc8ccc9nc(-c%10ccccc%10)oc9c8c7c6c5)nc(-c5cccc6ccccc56)n4)cc3)cc2)cc1]

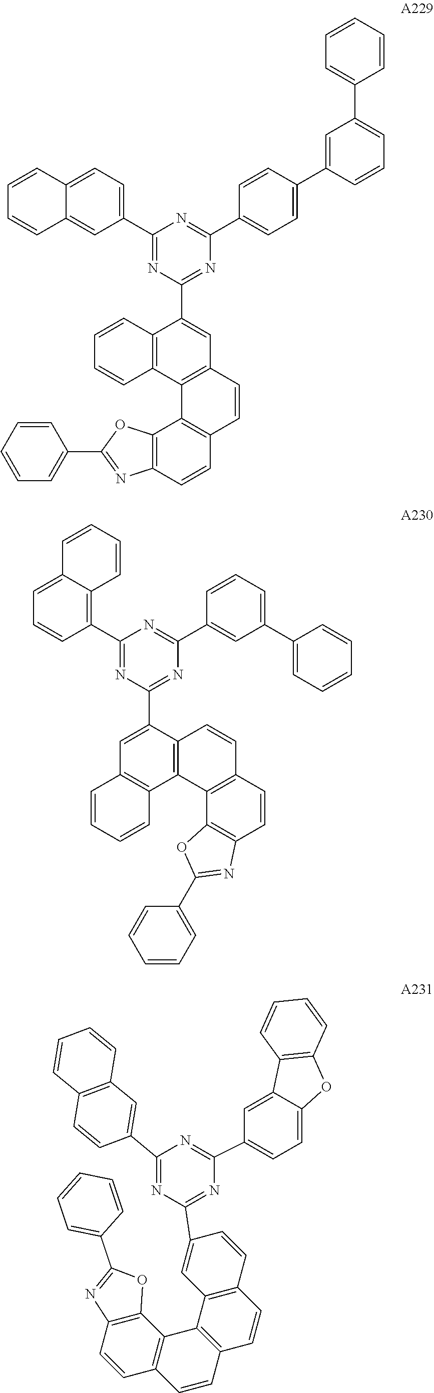 [SMILES: c1ccc(-c2cccc(-c3ccc(-c4nc(-c5ccc6ccccc6c5)nc(-c5cc6ccc7ccc8nc(-c9ccccc9)oc8c7c6c6ccccc56)n4)cc3)c2)cc1 ; c1ccc(-c2cccc(-c3nc(-c4cccc5ccccc45)nc(-c4cc5ccccc5c5c4ccc4ccc6nc(-c7ccccc7)oc6c45)n3)c2)cc1 ; c1ccc(-c2nc3ccc4ccc5ccc6ccc(-c7nc(-c8ccc9ccccc9c8)nc(-c8ccc9oc%10ccccc%10c9c8)n7)cc6c5c4c3o2)cc1]

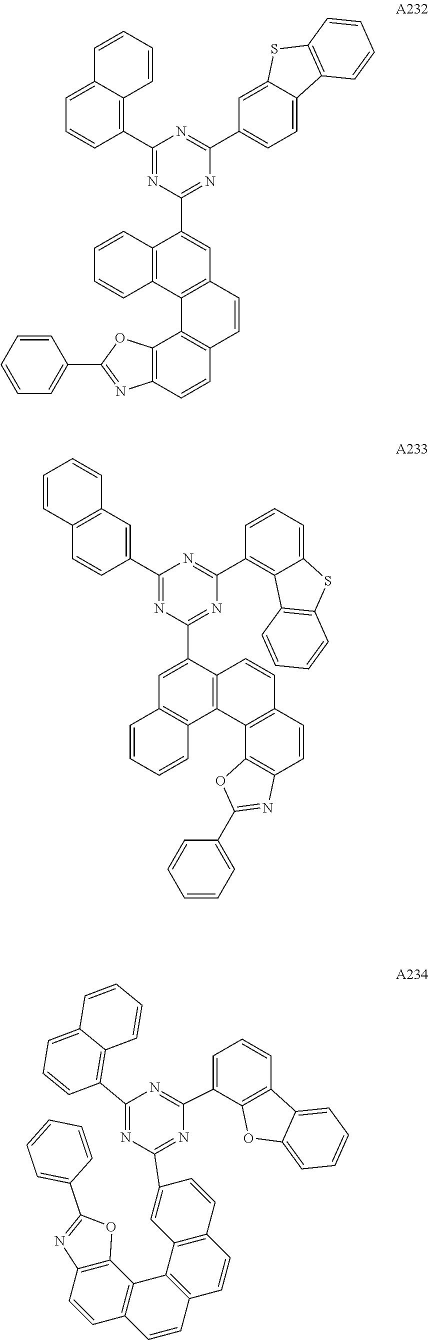 [SMILES: c1ccc(-c2nc3ccc4ccc5cc(-c6nc(-c7ccc8c(c7)sc7ccccc78)nc(-c7cccc8ccccc78)n6)c6ccccc6c5c4c3o2)cc1 ; c1ccc(-c2nc3ccc4ccc5c(-c6nc(-c7ccc8ccccc8c7)nc(-c7cccc8sc9ccccc9c78)n6)cc6ccccc6c5c4c3o2)cc1 ; c1ccc(-c2nc3ccc4ccc5ccc6ccc(-c7nc(-c8cccc9ccccc89)nc(-c8cccc9c8oc8ccccc89)n7)cc6c5c4c3o2)cc1]

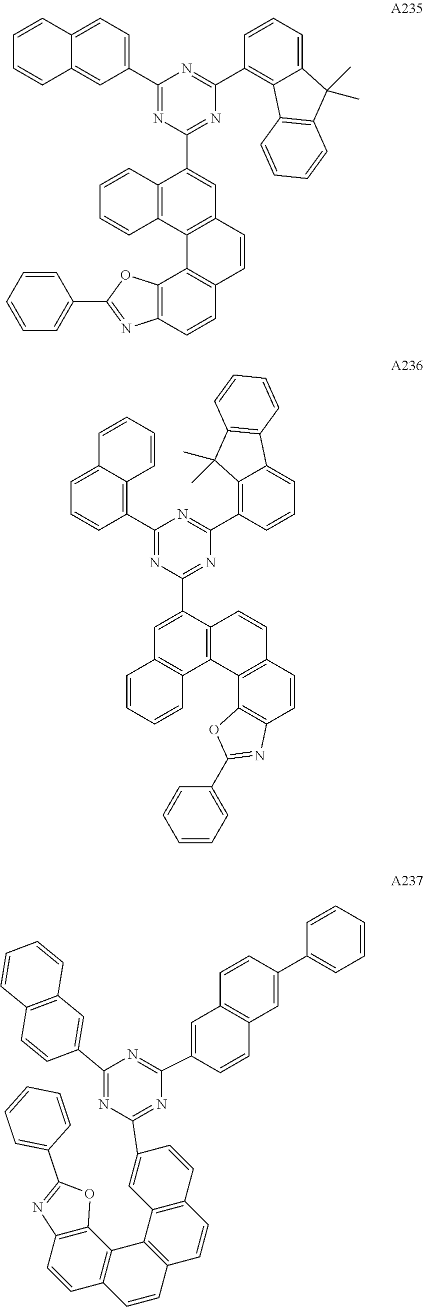 [SMILES: CC1(C)c2ccccc2-c2c(-c3nc(-c4ccc5ccccc5c4)nc(-c4cc5ccc6ccc7nc(-c8ccccc8)oc7c6c5c5ccccc45)n3)cccc21 ; CC1(C)c2ccccc2-c2cccc(-c3nc(-c4cccc5ccccc45)nc(-c4cc5ccccc5c5c4ccc4ccc6nc(-c7ccccc7)oc6c45)n3)c21 ; c1ccc(-c2ccc3cc(-c4nc(-c5ccc6ccccc6c5)nc(-c5ccc6ccc7ccc8ccc9nc(-c%10ccccc%10)oc9c8c7c6c5)n4)ccc3c2)cc1]

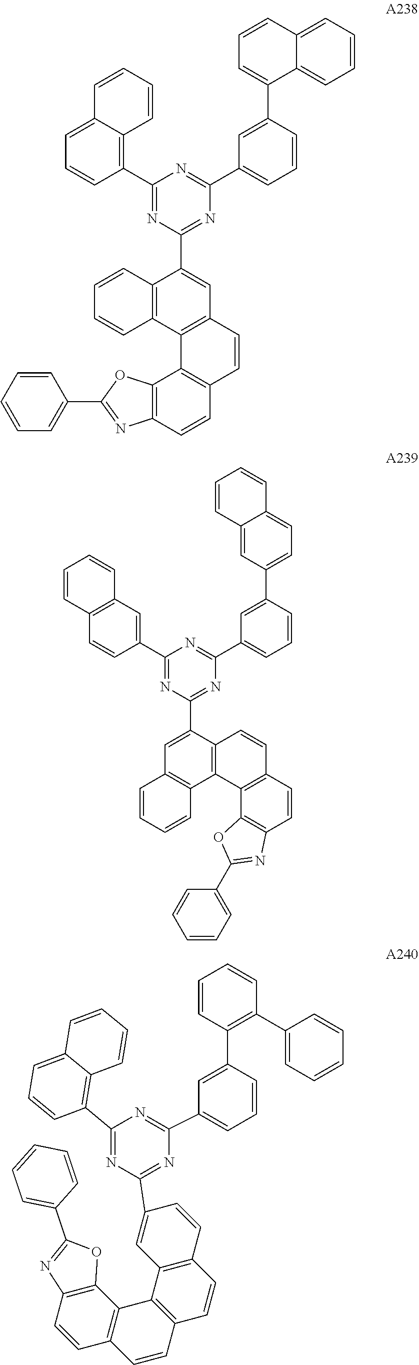

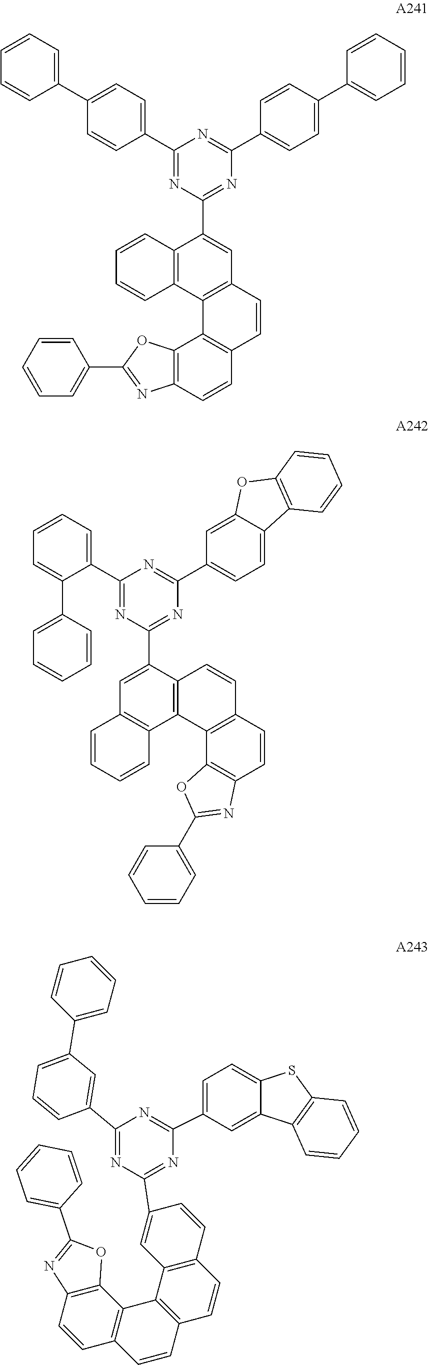 [SMILES: c1ccc(-c2ccc(-c3nc(-c4ccc(-c5ccccc5)cc4)nc(-c4cc5ccc6ccc7nc(-c8ccccc8)oc7c6c5c5ccccc45)n3)cc2)cc1 ; c1ccc(-c2nc3ccc4ccc5c(-c6nc(-c7ccc8c(c7)oc7ccccc78)nc(-c7ccccc7-c7ccccc7)n6)cc6ccccc6c5c4c3o2)cc1 ; c1ccc(-c2cccc(-c3nc(-c4ccc5sc6ccccc6c5c4)nc(-c4ccc5ccc6ccc7ccc8nc(-c9ccccc9)oc8c7c6c5c4)n3)c2)cc1]

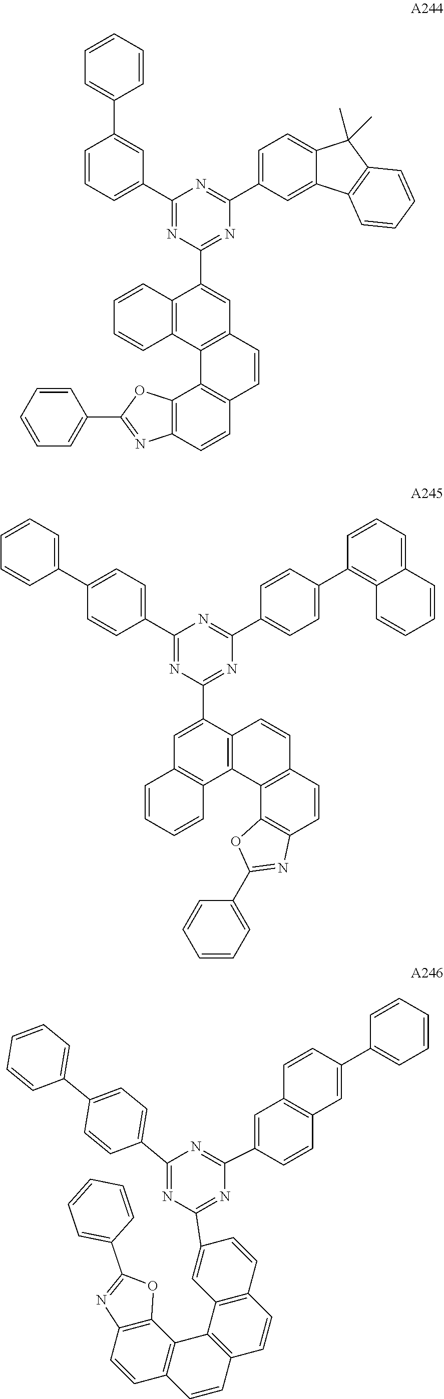 [SMILES: CC1(C)c2ccccc2-c2cc(-c3nc(-c4cccc(-c5ccccc5)c4)nc(-c4cc5ccc6ccc7nc(-c8ccccc8)oc7c6c5c5ccccc45)n3)ccc21 ; c1ccc(-c2ccc(-c3nc(-c4ccc(-c5cccc6ccccc56)cc4)nc(-c4cc5ccccc5c5c4ccc4ccc6nc(-c7ccccc7)oc6c45)n3)cc2)cc1 ; c1ccc(-c2ccc(-c3nc(-c4ccc5cc(-c6ccccc6)ccc5c4)nc(-c4ccc5ccc6ccc7ccc8nc(-c9ccccc9)oc8c7c6c5c4)n3)cc2)cc1]

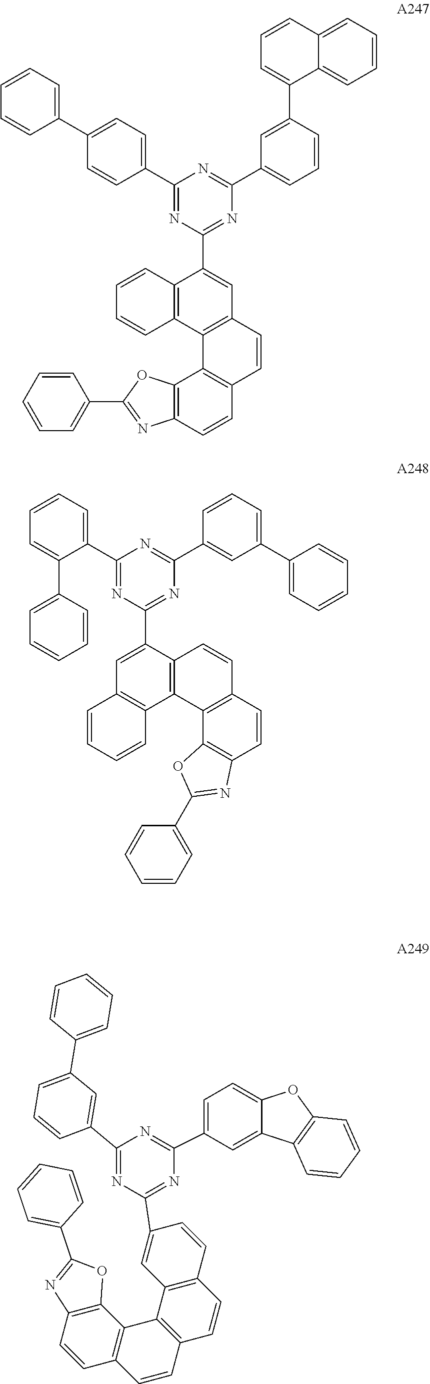 [SMILES: c1ccc(-c2ccc(-c3nc(-c4cccc(-c5cccc6ccccc56)c4)nc(-c4cc5ccc6ccc7nc(-c8ccccc8)oc7c6c5c5ccccc45)n3)cc2)cc1 ; c1ccc(-c2cccc(-c3nc(-c4ccccc4-c4ccccc4)nc(-c4cc5ccccc5c5c4ccc4ccc6nc(-c7ccccc7)oc6c45)n3)c2)cc1 ; c1ccc(-c2cccc(-c3nc(-c4ccc5oc6ccccc6c5c4)nc(-c4ccc5ccc6ccc7ccc8nc(-c9ccccc9)oc8c7c6c5c4)n3)c2)cc1]

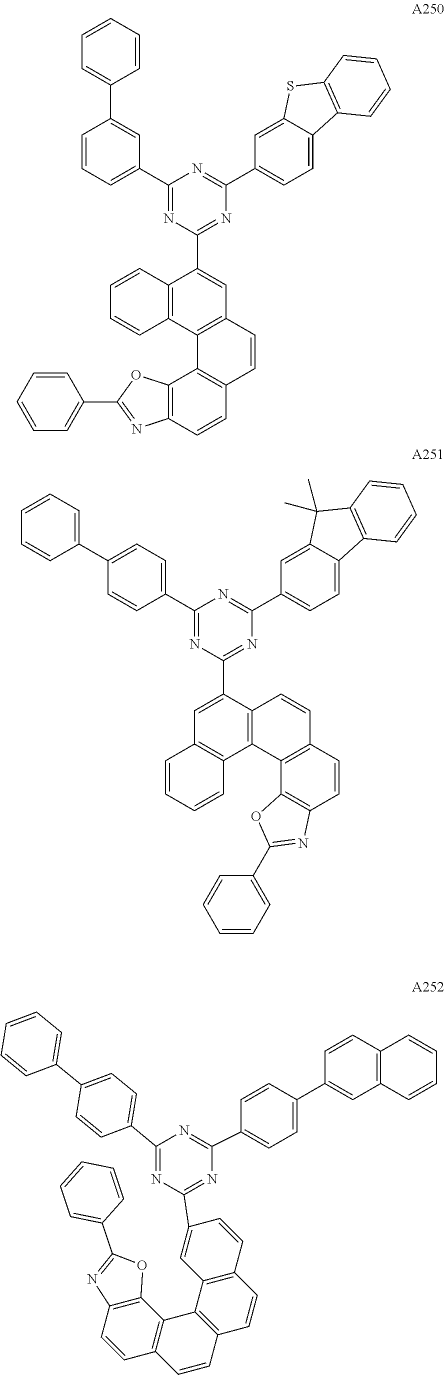 [SMILES: c1ccc(-c2cccc(-c3nc(-c4ccc5c(c4)sc4ccccc45)nc(-c4cc5ccc6ccc7nc(-c8ccccc8)oc7c6c5c5ccccc45)n3)c2)cc1 ; CC1(C)c2ccccc2-c2ccc(-c3nc(-c4ccc(-c5ccccc5)cc4)nc(-c4cc5ccccc5c5c4ccc4ccc6nc(-c7ccccc7)oc6c45)n3)cc21 ; c1ccc(-c2ccc(-c3nc(-c4ccc(-c5ccc6ccccc6c5)cc4)nc(-c4ccc5ccc6ccc7ccc8nc(-c9ccccc9)oc8c7c6c5c4)n3)cc2)cc1]

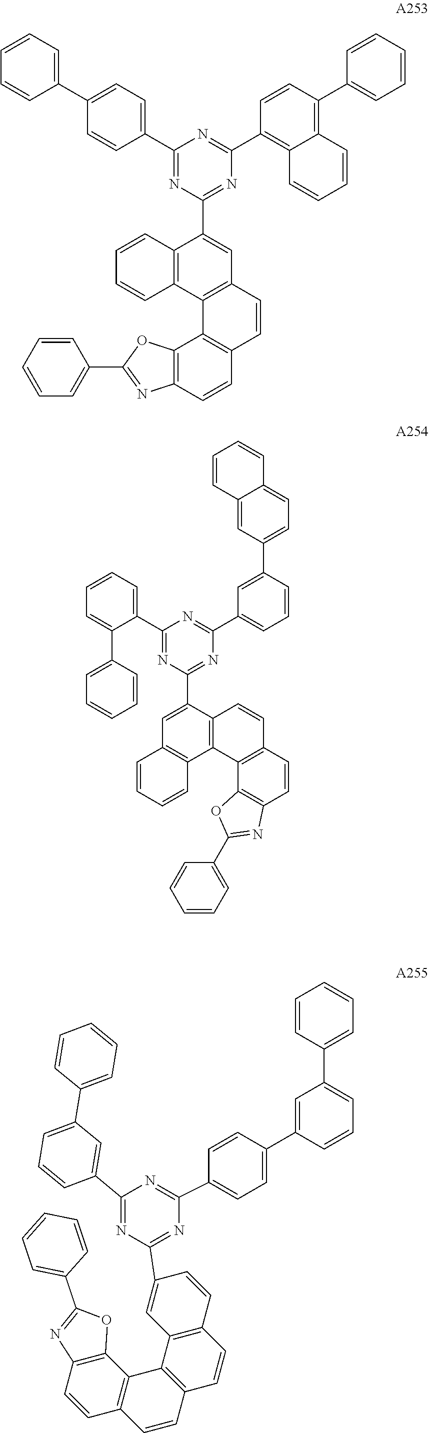 [SMILES: c1ccc(-c2ccc(-c3nc(-c4ccc(-c5ccccc5)c5ccccc45)nc(-c4cc5ccc6ccc7nc(-c8ccccc8)oc7c6c5c5ccccc45)n3)cc2)cc1 ; c1ccc(-c2nc3ccc4ccc5c(-c6nc(-c7cccc(-c8ccc9ccccc9c8)c7)nc(-c7ccccc7-c7ccccc7)n6)cc6ccccc6c5c4c3o2)cc1 ; c1ccc(-c2cccc(-c3ccc(-c4nc(-c5cccc(-c6ccccc6)c5)nc(-c5ccc6ccc7ccc8ccc9nc(-c%10ccccc%10)oc9c8c7c6c5)n4)cc3)c2)cc1]

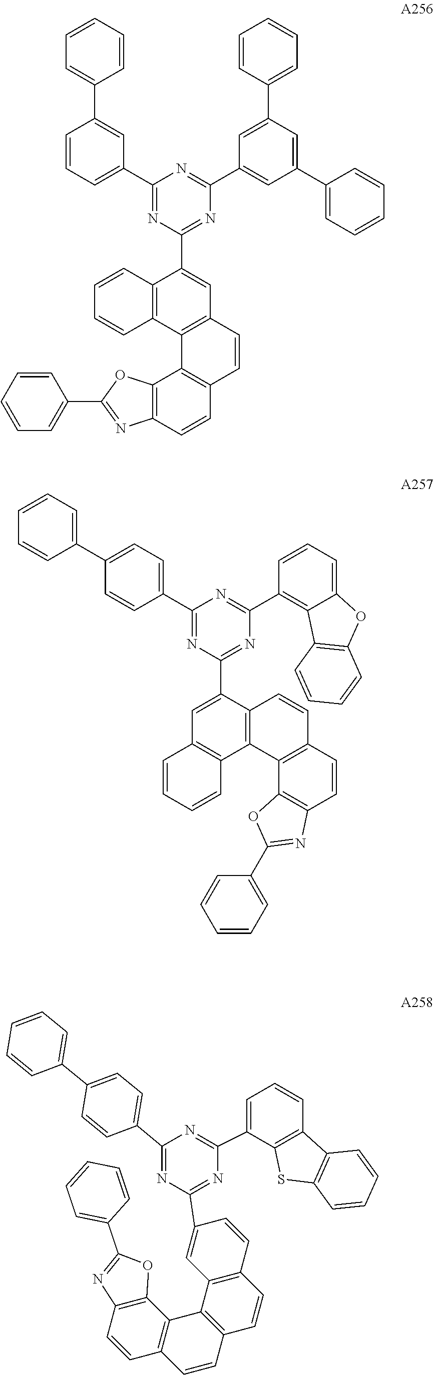 [SMILES: c1ccc(-c2cccc(-c3nc(-c4cc(-c5ccccc5)cc(-c5ccccc5)c4)nc(-c4cc5ccc6ccc7nc(-c8ccccc8)oc7c6c5c5ccccc45)n3)c2)cc1 ; c1ccc(-c2ccc(-c3nc(-c4cc5ccccc5c5c4ccc4ccc6nc(-c7ccccc7)oc6c45)nc(-c4cccc5oc6ccccc6c45)n3)cc2)cc1 ; c1ccc(-c2ccc(-c3nc(-c4ccc5ccc6ccc7ccc8nc(-c9ccccc9)oc8c7c6c5c4)nc(-c4cccc5c4sc4ccccc45)n3)cc2)cc1]

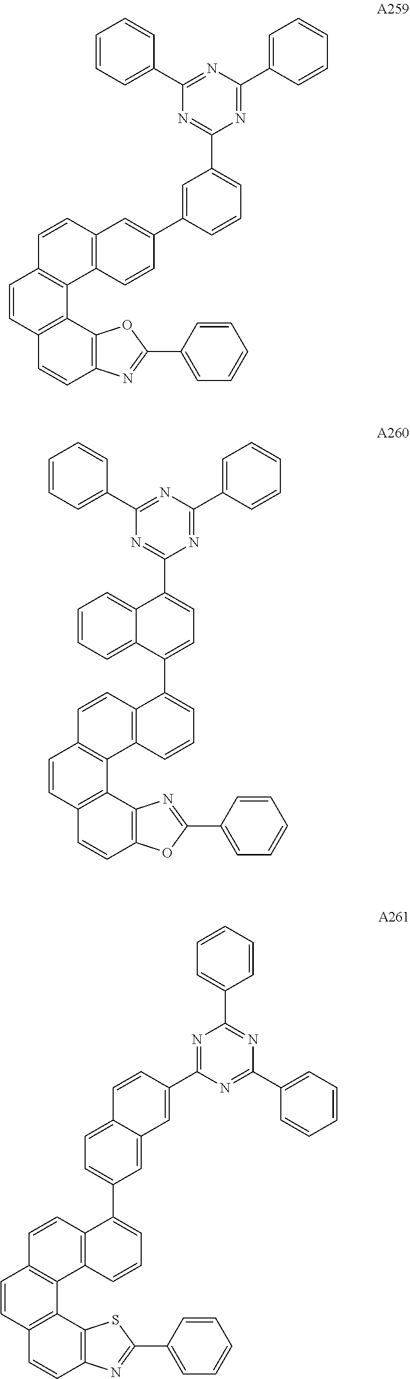 [SMILES: c1ccc(-c2nc(-c3ccccc3)nc(-c3cccc(-c4ccc5c(ccc6ccc7ccc8nc(-c9ccccc9)oc8c7c65)c4)c3)n2)cc1 ; c1ccc(-c2nc(-c3ccccc3)nc(-c3ccc(-c4cccc5c4ccc4ccc6ccc7oc(-c8ccccc8)nc7c6c45)c4ccccc34)n2)cc1 ; c1ccc(-c2nc(-c3ccccc3)nc(-c3ccc4ccc(-c5cccc6c5ccc5ccc7ccc8nc(-c9ccccc9)sc8c7c56)cc4c3)n2)cc1]

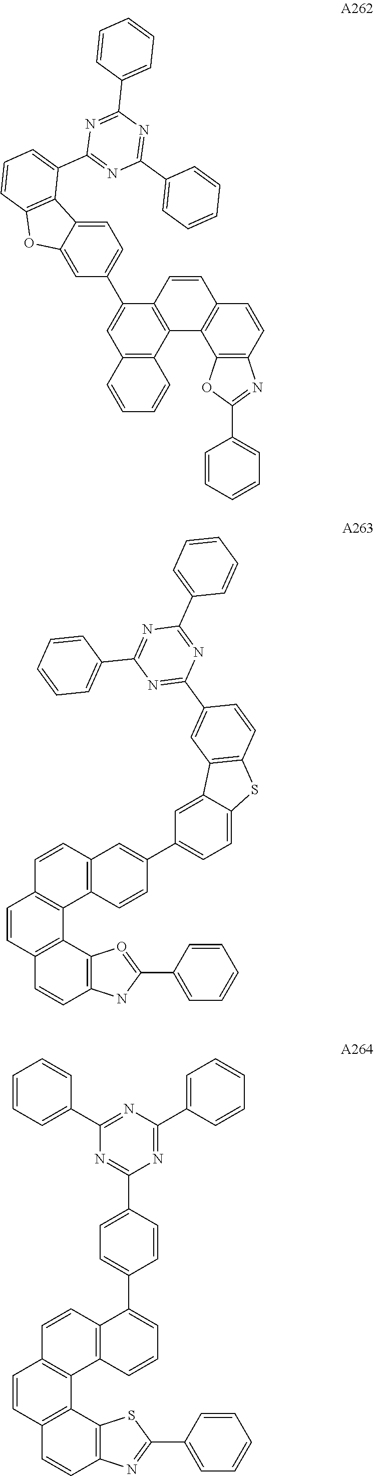

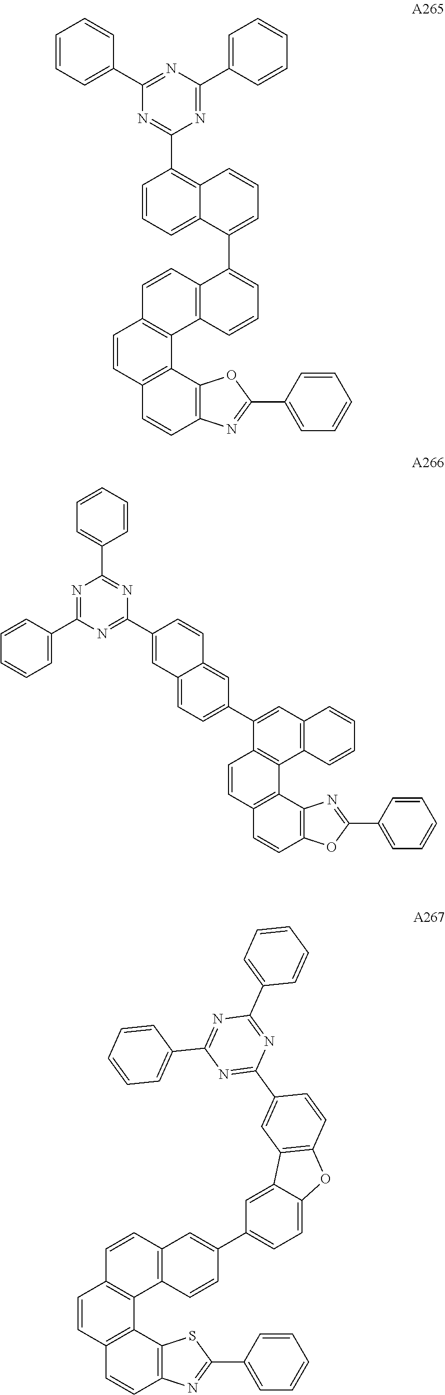 [SMILES: c1ccc(-c2nc(-c3ccccc3)nc(-c3cccc4c(-c5cccc6c5ccc5ccc7ccc8nc(-c9ccccc9)oc8c7c56)cccc34)n2)cc1 ; c1ccc(-c2nc(-c3ccccc3)nc(-c3ccc4cc(-c5cc6ccccc6c6c5ccc5ccc7oc(-c8ccccc8)nc7c56)ccc4c3)n2)cc1 ; c1ccc(-c2nc(-c3ccccc3)nc(-c3ccc4oc5ccc(-c6ccc7c(ccc8ccc9ccc%10nc(-c%11ccccc%11)sc%10c9c87)c6)cc5c4c3)n2)cc1]

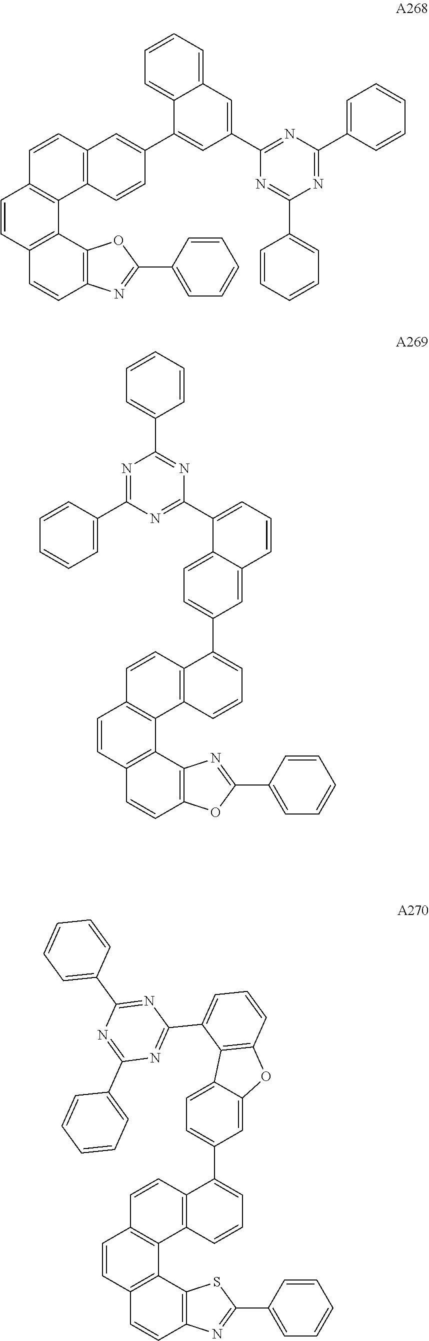 [SMILES: c1ccc(-c2nc(-c3ccccc3)nc(-c3cc(-c4ccc5c(ccc6ccc7ccc8nc(-c9ccccc9)oc8c7c65)c4)c4ccccc4c3)n2)cc1 ; c1ccc(-c2nc(-c3ccccc3)nc(-c3cccc4cc(-c5cccc6c5ccc5ccc7ccc8oc(-c9ccccc9)nc8c7c56)ccc34)n2)cc1 ; c1ccc(-c2nc(-c3ccccc3)nc(-c3cccc4oc5cc(-c6cccc7c6ccc6ccc8ccc9nc(-c%10ccccc%10)sc9c8c67)ccc5c34)n2)cc1]

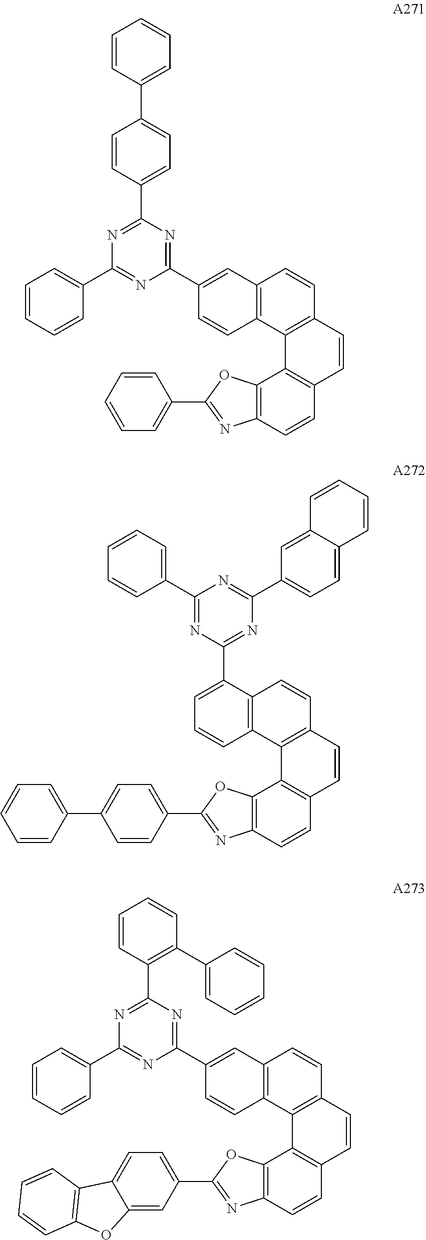 [SMILES: c1ccc(-c2ccc(-c3nc(-c4ccccc4)nc(-c4ccc5c(ccc6ccc7ccc8nc(-c9ccccc9)oc8c7c65)c4)n3)cc2)cc1 ; c1ccc(-c2ccc(-c3nc4ccc5ccc6ccc7c(-c8nc(-c9ccccc9)nc(-c9ccc%10ccccc%10c9)n8)cccc7c6c5c4o3)cc2)cc1 ; c1ccc(-c2nc(-c3ccc4c(ccc5ccc6ccc7nc(-c8ccc9c(c8)oc8ccccc89)oc7c6c54)c3)nc(-c3ccccc3-c3ccccc3)n2)cc1]

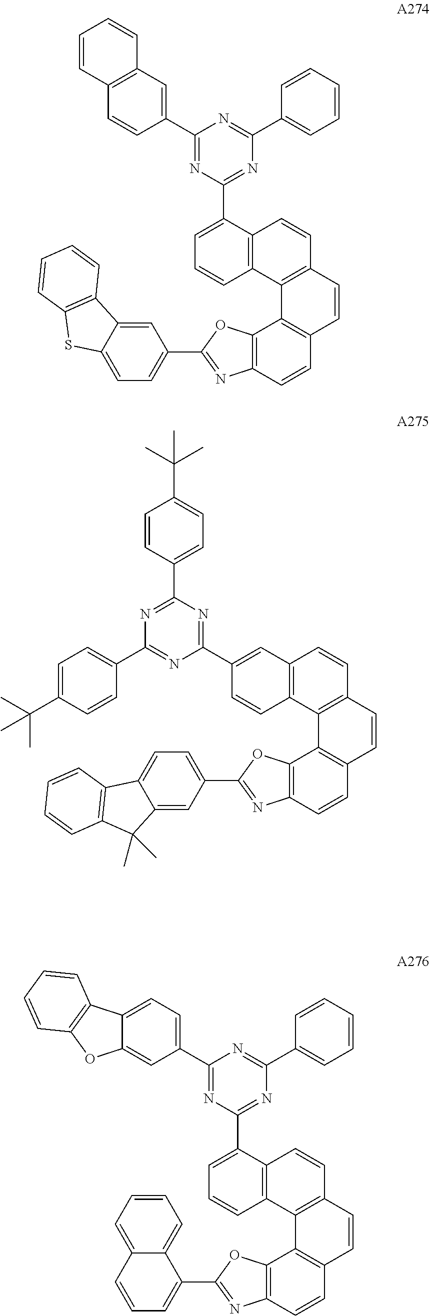 [SMILES: c1ccc(-c2nc(-c3ccc4ccccc4c3)nc(-c3cccc4c3ccc3ccc5ccc6nc(-c7ccc8sc9ccccc9c8c7)oc6c5c34)n2)cc1 ; CC(C)(C)c1ccc(-c2nc(-c3ccc(C(C)(C)C)cc3)nc(-c3ccc4c(ccc5ccc6ccc7nc(-c8ccc9c(c8)C(C)(C)c8ccccc8-9)oc7c6c54)c3)n2)cc1 ; c1ccc(-c2nc(-c3ccc4c(c3)oc3ccccc34)nc(-c3cccc4c3ccc3ccc5ccc6nc(-c7cccc8ccccc78)oc6c5c34)n2)cc1]

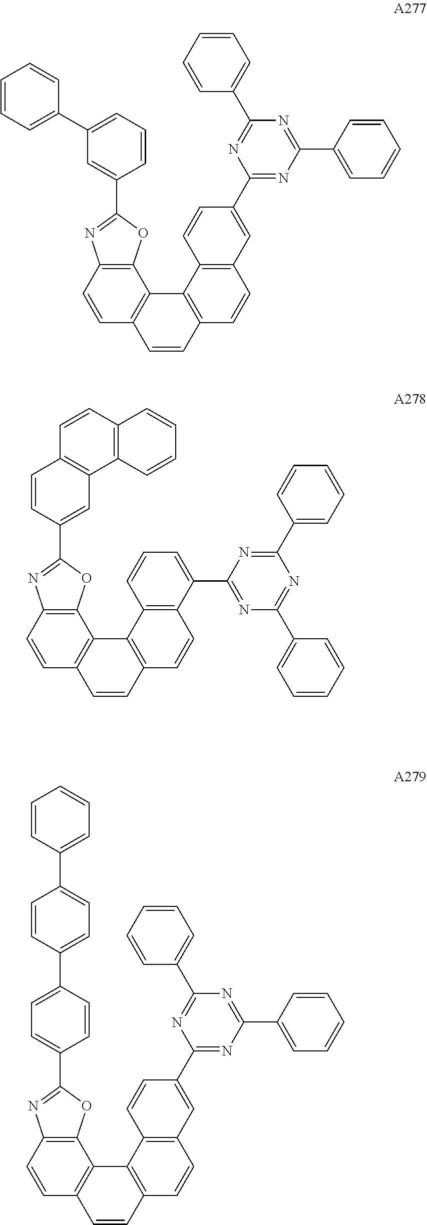 [SMILES: c1ccc(-c2cccc(-c3nc4ccc5ccc6ccc7cc(-c8nc(-c9ccccc9)nc(-c9ccccc9)n8)ccc7c6c5c4o3)c2)cc1 ; c1ccc(-c2nc(-c3ccccc3)nc(-c3cccc4c3ccc3ccc5ccc6nc(-c7ccc8ccc9ccccc9c8c7)oc6c5c34)n2)cc1 ; c1ccc(-c2ccc(-c3ccc(-c4nc5ccc6ccc7ccc8cc(-c9nc(-c%10ccccc%10)nc(-c%10ccccc%10)n9)ccc8c7c6c5o4)cc3)cc2)cc1]

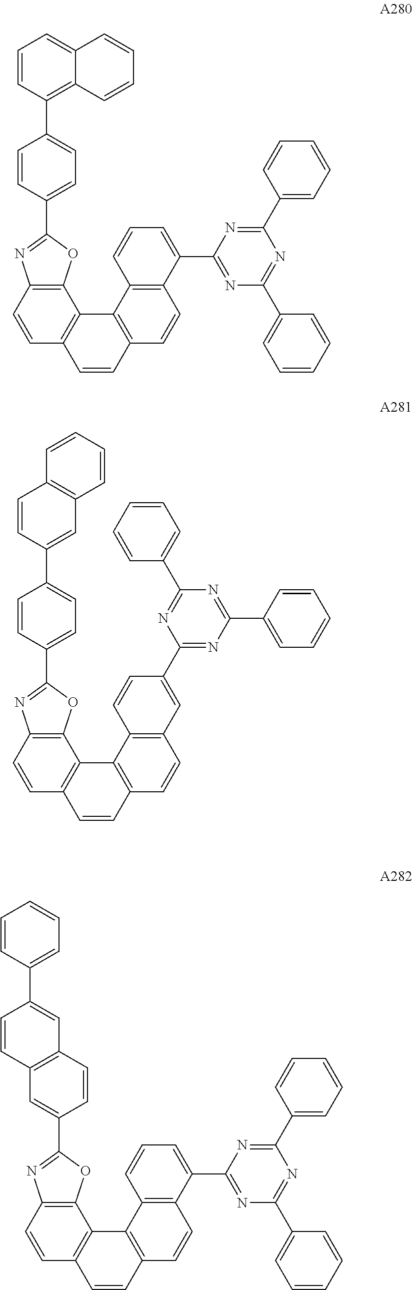 [SMILES: c1ccc(-c2nc(-c3ccccc3)nc(-c3cccc4c3ccc3ccc5ccc6nc(-c7ccc(-c8cccc9ccccc89)cc7)oc6c5c34)n2)cc1 ; c1ccc(-c2nc(-c3ccccc3)nc(-c3ccc4c(ccc5ccc6ccc7nc(-c8ccc(-c9ccc%10ccccc%10c9)cc8)oc7c6c54)c3)n2)cc1 ; c1ccc(-c2ccc3cc(-c4nc5ccc6ccc7ccc8c(-c9nc(-c%10ccccc%10)nc(-c%10ccccc%10)n9)cccc8c7c6c5o4)ccc3c2)cc1]

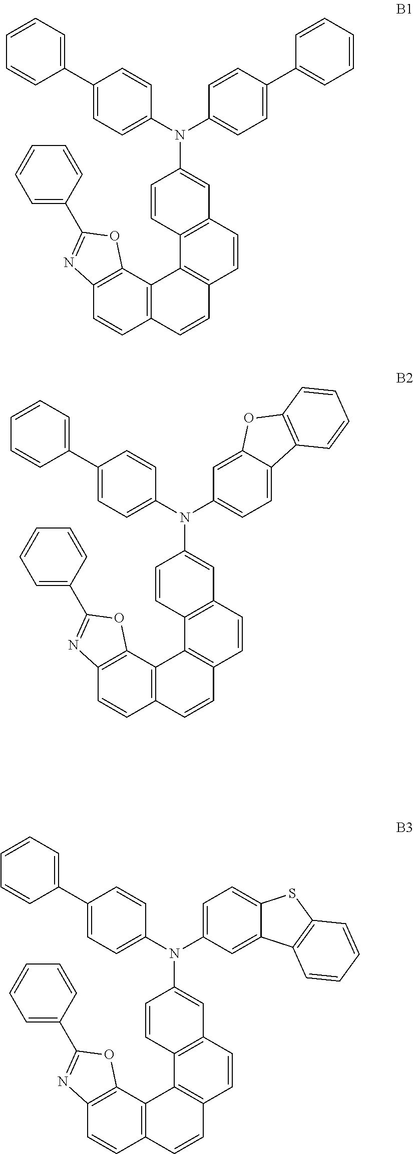 [SMILES: c1ccc(-c2ccc(N(c3ccc(-c4ccccc4)cc3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc4c(c3)oc3ccccc34)cc2)cc1 ; c1ccc(-c2ccc(N(c3ccc4c(ccc5ccc6ccc7nc(-c8ccccc8)oc7c6c54)c3)c3ccc4sc5ccccc5c4c3)cc2)cc1]

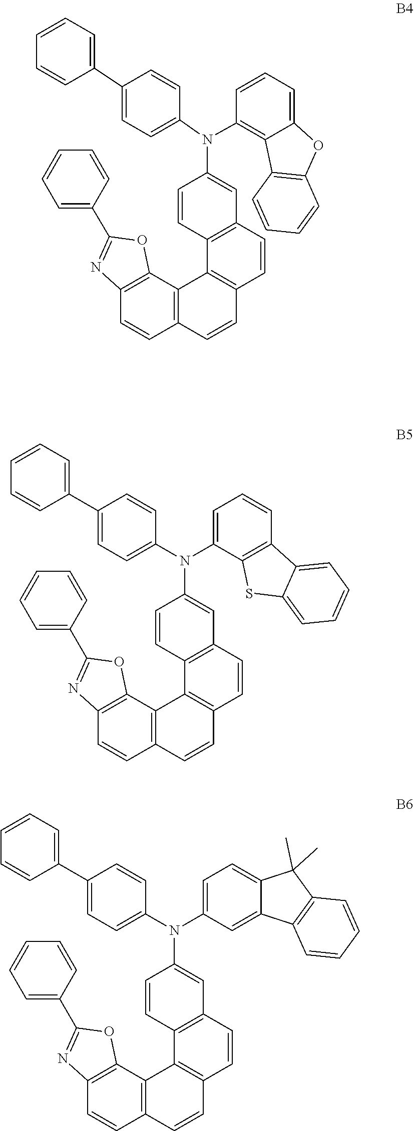 [SMILES: c1ccc(-c2ccc(N(c3ccc4c(ccc5ccc6ccc7nc(-c8ccccc8)oc7c6c54)c3)c3cccc4oc5ccccc5c34)cc2)cc1 ; c1ccc(-c2ccc(N(c3ccc4c(ccc5ccc6ccc7nc(-c8ccccc8)oc7c6c54)c3)c3cccc4c3sc3ccccc34)cc2)cc1 ; CC1(C)c2ccccc2-c2cc(N(c3ccc(-c4ccccc4)cc3)c3ccc4c(ccc5ccc6ccc7nc(-c8ccccc8)oc7c6c54)c3)ccc21]

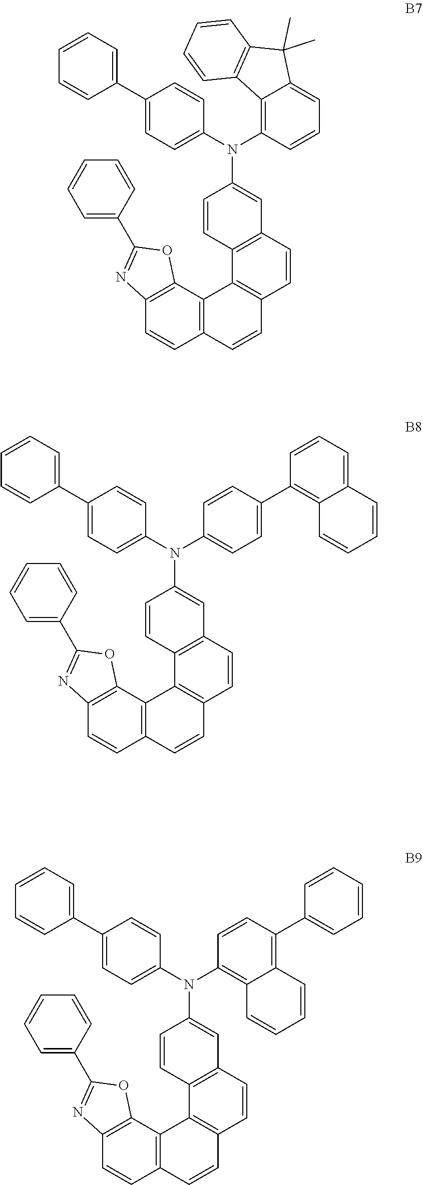 [SMILES: CC1(C)c2ccccc2-c2c(N(c3ccc(-c4ccccc4)cc3)c3ccc4c(ccc5ccc6ccc7nc(-c8ccccc8)oc7c6c54)c3)cccc21 ; c1ccc(-c2ccc(N(c3ccc(-c4cccc5ccccc45)cc3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc(-c4ccccc4)c4ccccc34)cc2)cc1]

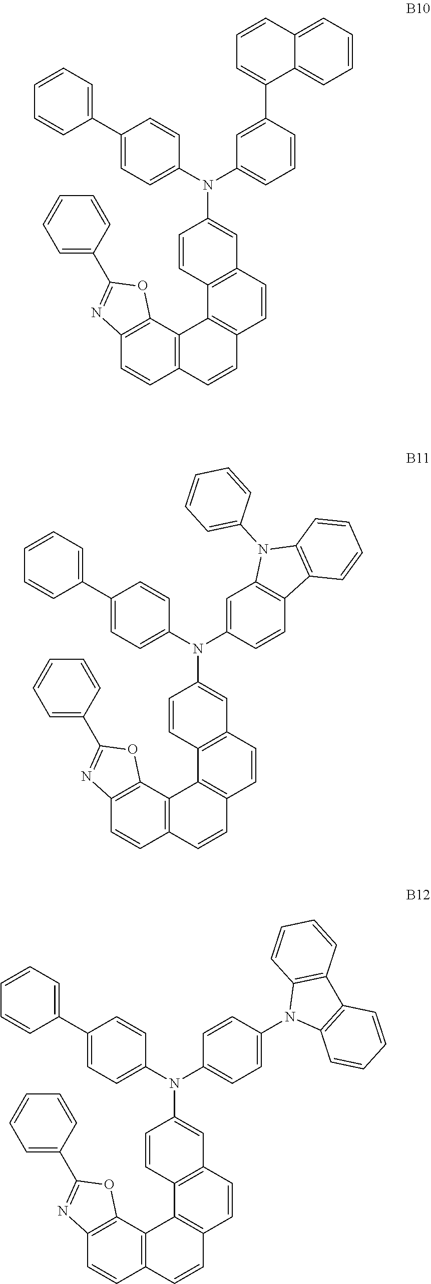 [SMILES: c1ccc(-c2ccc(N(c3cccc(-c4cccc5ccccc45)c3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc4c5ccccc5n(-c5ccccc5)c4c3)cc2)cc1 ; c1ccc(-c2ccc(N(c3ccc(-n4c5ccccc5c5ccccc54)cc3)c3ccc4c(ccc5ccc6ccc7nc(-c8ccccc8)oc7c6c54)c3)cc2)cc1]

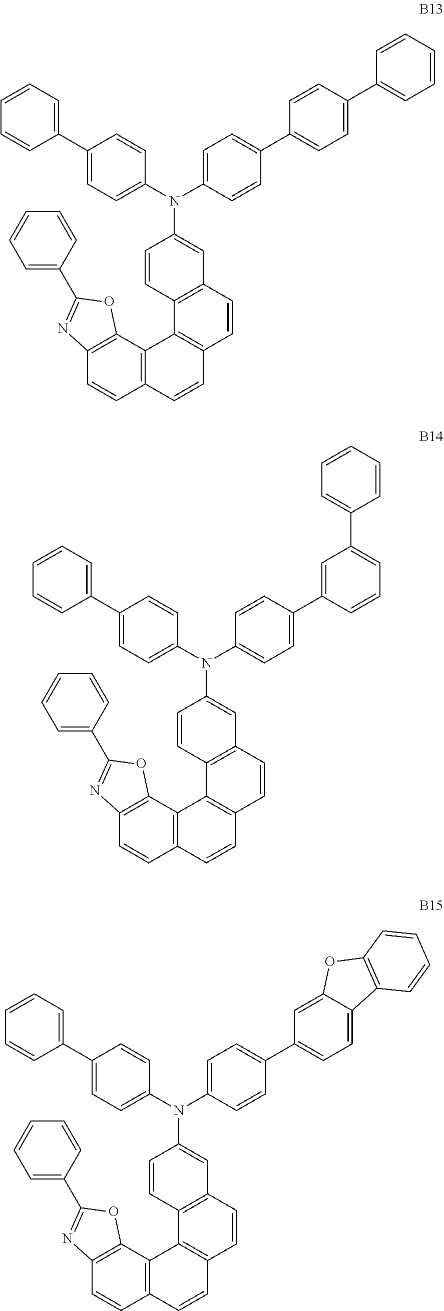 [SMILES: c1ccc(-c2ccc(-c3ccc(N(c4ccc(-c5ccccc5)cc4)c4ccc5c(ccc6ccc7ccc8nc(-c9ccccc9)oc8c7c65)c4)cc3)cc2)cc1 ; c1ccc(-c2ccc(N(c3ccc(-c4cccc(-c5ccccc5)c4)cc3)c3ccc4c(ccc5ccc6ccc7nc(-c8ccccc8)oc7c6c54)c3)cc2)cc1 ; c1ccc(-c2ccc(N(c3ccc(-c4ccc5c(c4)oc4ccccc45)cc3)c3ccc4c(ccc5ccc6ccc7nc(-c8ccccc8)oc7c6c54)c3)cc2)cc1]

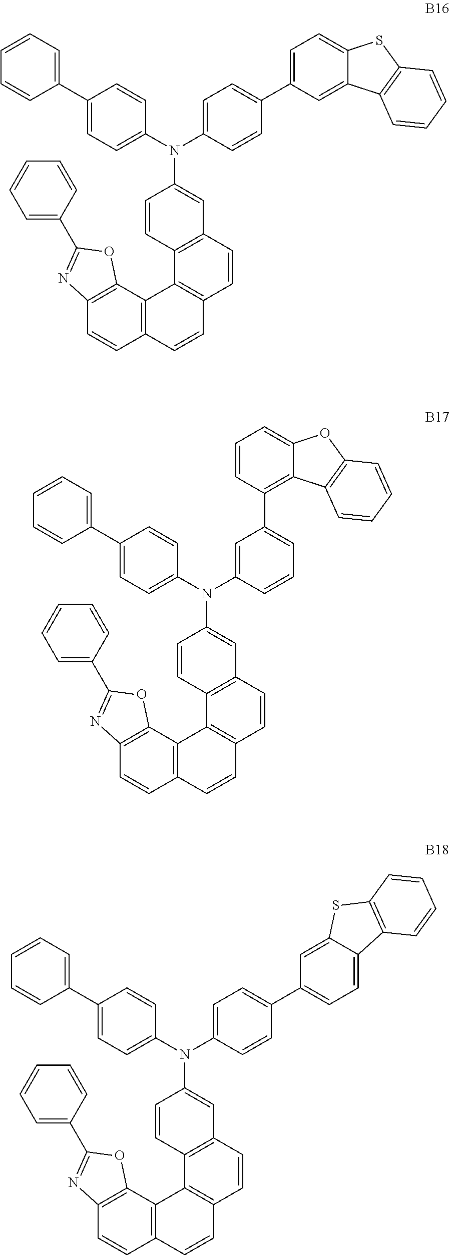 [SMILES: c1ccc(-c2ccc(N(c3ccc(-c4ccc5sc6ccccc6c5c4)cc3)c3ccc4c(ccc5ccc6ccc7nc(-c8ccccc8)oc7c6c54)c3)cc2)cc1 ; c1ccc(-c2ccc(N(c3cccc(-c4cccc5oc6ccccc6c45)c3)c3ccc4c(ccc5ccc6ccc7nc(-c8ccccc8)oc7c6c54)c3)cc2)cc1 ; c1ccc(-c2ccc(N(c3ccc(-c4ccc5c(c4)sc4ccccc45)cc3)c3ccc4c(ccc5ccc6ccc7nc(-c8ccccc8)oc7c6c54)c3)cc2)cc1]

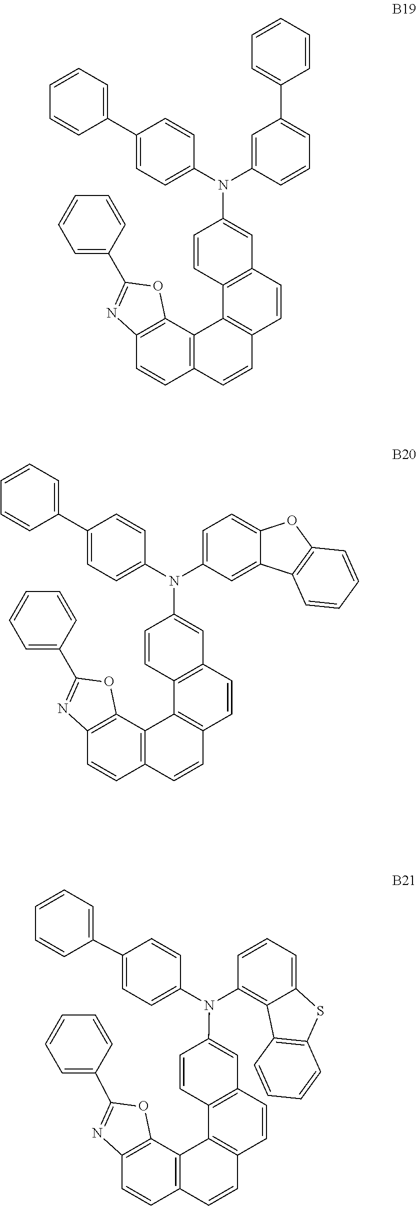 [SMILES: c1ccc(-c2ccc(N(c3cccc(-c4ccccc4)c3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc4oc5ccccc5c4c3)cc2)cc1 ; c1ccc(-c2ccc(N(c3ccc4c(ccc5ccc6ccc7nc(-c8ccccc8)oc7c6c54)c3)c3cccc4sc5ccccc5c34)cc2)cc1]

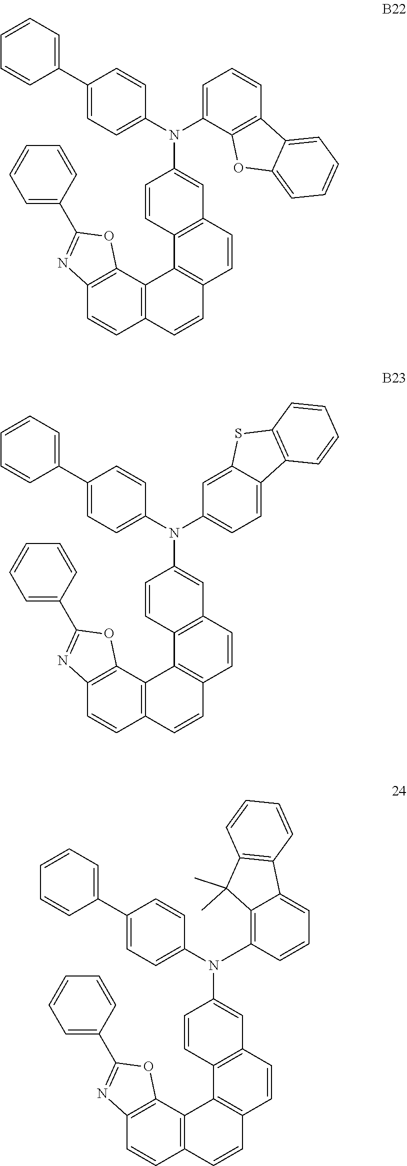 [SMILES: c1ccc(-c2ccc(N(c3ccc4c(ccc5ccc6ccc7nc(-c8ccccc8)oc7c6c54)c3)c3cccc4c3oc3ccccc34)cc2)cc1 ; c1ccc(-c2ccc(N(c3ccc4c(ccc5ccc6ccc7nc(-c8ccccc8)oc7c6c54)c3)c3ccc4c(c3)sc3ccccc34)cc2)cc1 ; CC1(C)c2ccccc2-c2cccc(N(c3ccc(-c4ccccc4)cc3)c3ccc4c(ccc5ccc6ccc7nc(-c8ccccc8)oc7c6c54)c3)c21]

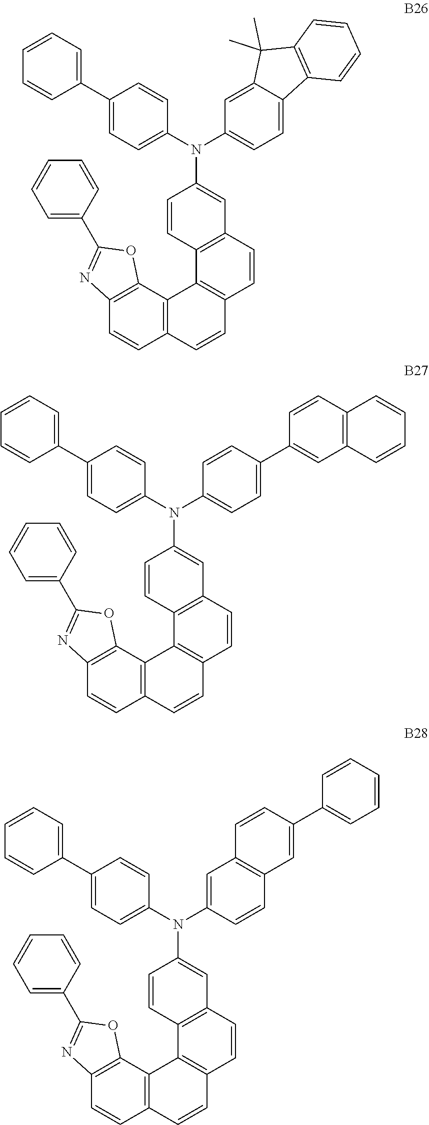 [SMILES: CC1(C)c2ccccc2-c2ccc(N(c3ccc(-c4ccccc4)cc3)c3ccc4c(ccc5ccc6ccc7nc(-c8ccccc8)oc7c6c54)c3)cc21 ; c1ccc(-c2ccc(N(c3ccc(-c4ccc5ccccc5c4)cc3)c3ccc4c(ccc5ccc6ccc7nc(-c8ccccc8)oc7c6c54)c3)cc2)cc1 ; c1ccc(-c2ccc(N(c3ccc4cc(-c5ccccc5)ccc4c3)c3ccc4c(ccc5ccc6ccc7nc(-c8ccccc8)oc7c6c54)c3)cc2)cc1]

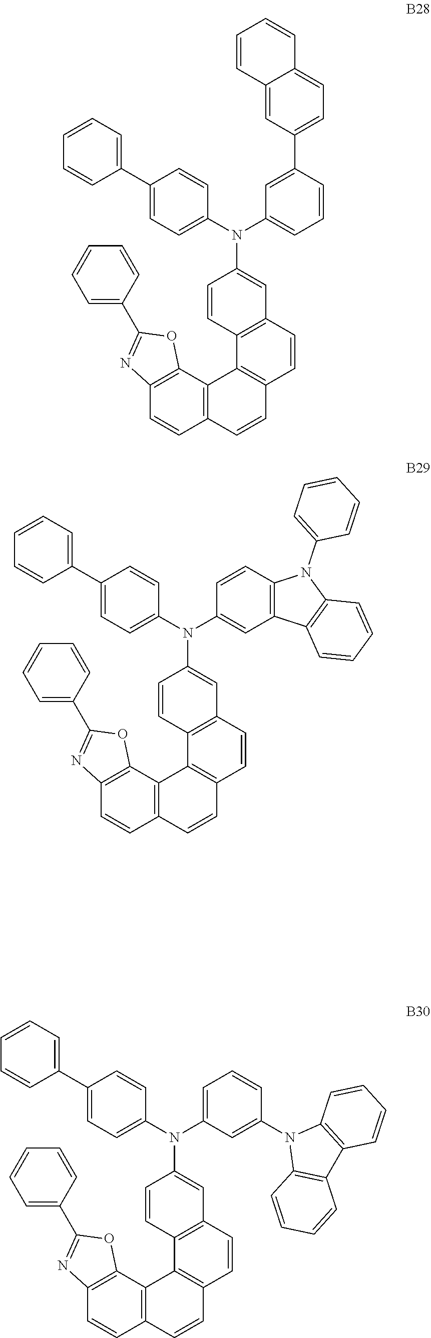 [SMILES: c1ccc(-c2ccc(N(c3cccc(-c4ccc5ccccc5c4)c3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc4c(c3)c3ccccc3n4-c3ccccc3)cc2)cc1 ; c1ccc(-c2ccc(N(c3cccc(-n4c5ccccc5c5ccccc54)c3)c3ccc4c(ccc5ccc6ccc7nc(-c8ccccc8)oc7c6c54)c3)cc2)cc1]

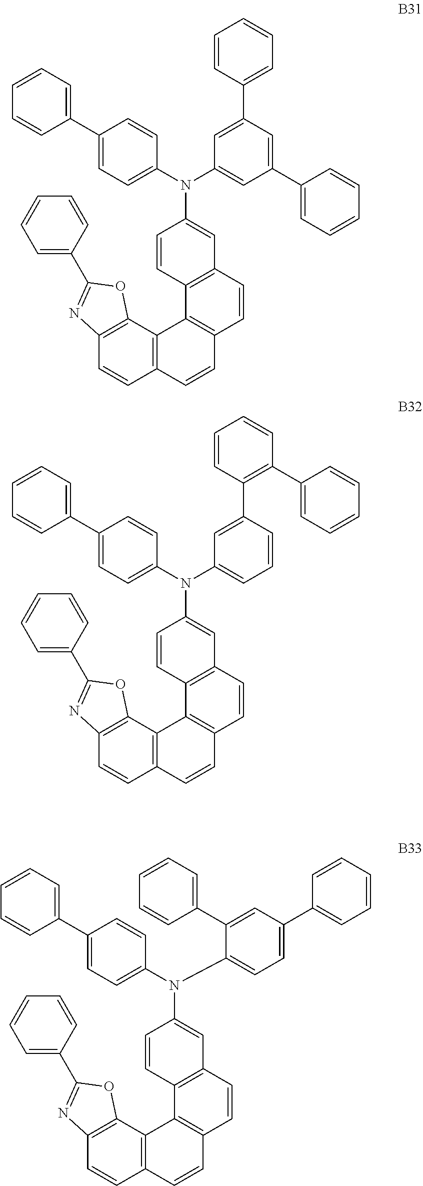 [SMILES: c1ccc(-c2ccc(N(c3cc(-c4ccccc4)cc(-c4ccccc4)c3)c3ccc4c(ccc5ccc6ccc7nc(-c8ccccc8)oc7c6c54)c3)cc2)cc1 ; c1ccc(-c2ccc(N(c3cccc(-c4ccccc4-c4ccccc4)c3)c3ccc4c(ccc5ccc6ccc7nc(-c8ccccc8)oc7c6c54)c3)cc2)cc1 ; c1ccc(-c2ccc(N(c3ccc4c(ccc5ccc6ccc7nc(-c8ccccc8)oc7c6c54)c3)c3ccc(-c4ccccc4)cc3-c3ccccc3)cc2)cc1]

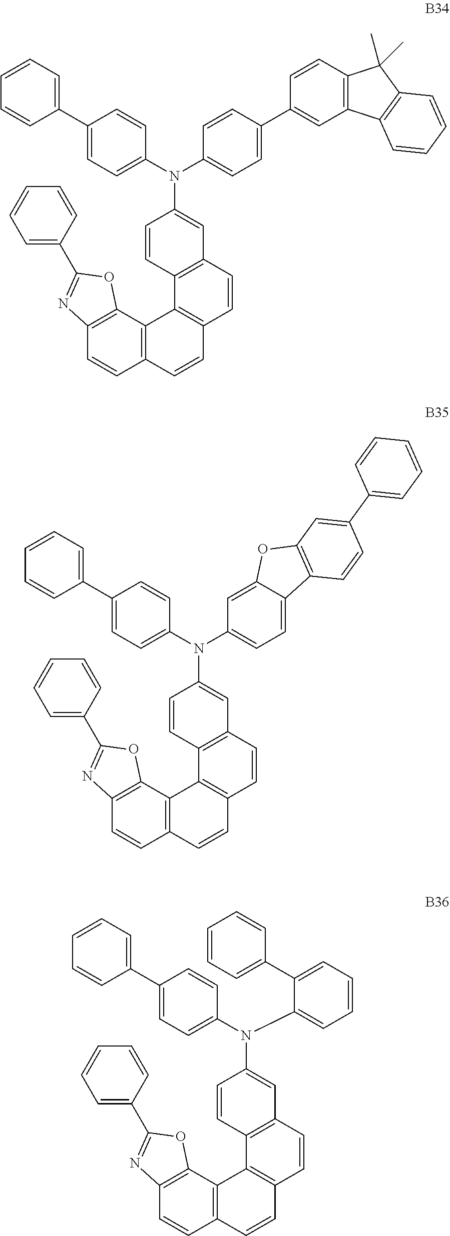 [SMILES: CC1(C)c2ccccc2-c2cc(-c3ccc(N(c4ccc(-c5ccccc5)cc4)c4ccc5c(ccc6ccc7ccc8nc(-c9ccccc9)oc8c7c65)c4)cc3)ccc21 ; c1ccc(-c2ccc(N(c3ccc4c(ccc5ccc6ccc7nc(-c8ccccc8)oc7c6c54)c3)c3ccc4c(c3)oc3cc(-c5ccccc5)ccc34)cc2)cc1 ; c1ccc(-c2ccc(N(c3ccc4c(ccc5ccc6ccc7nc(-c8ccccc8)oc7c6c54)c3)c3ccccc3-c3ccccc3)cc2)cc1]

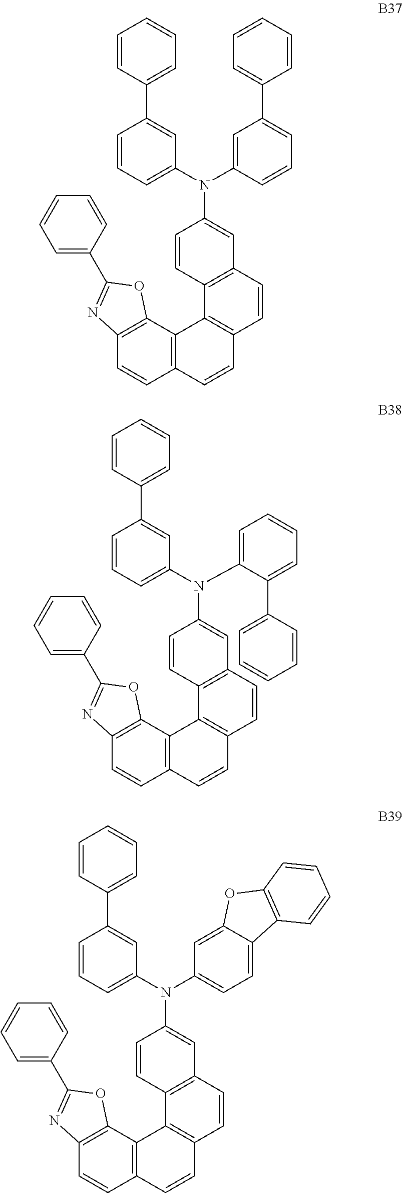

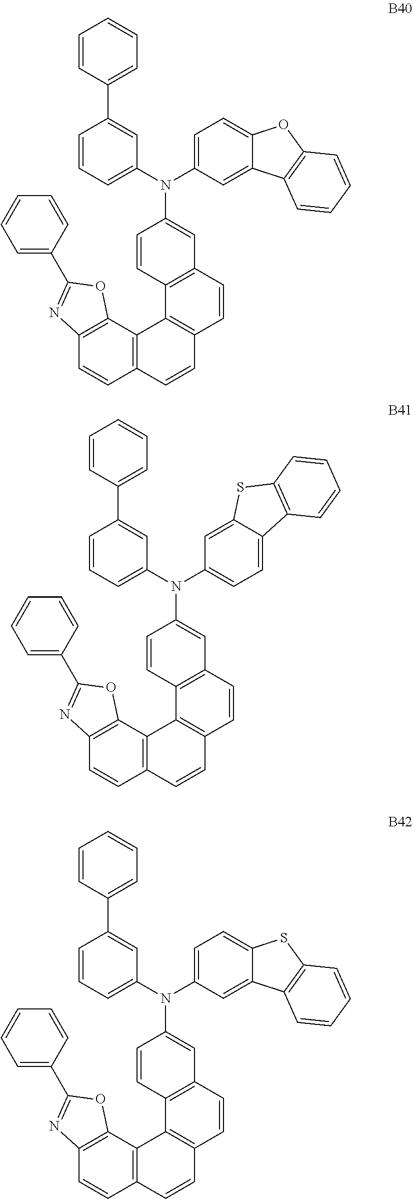

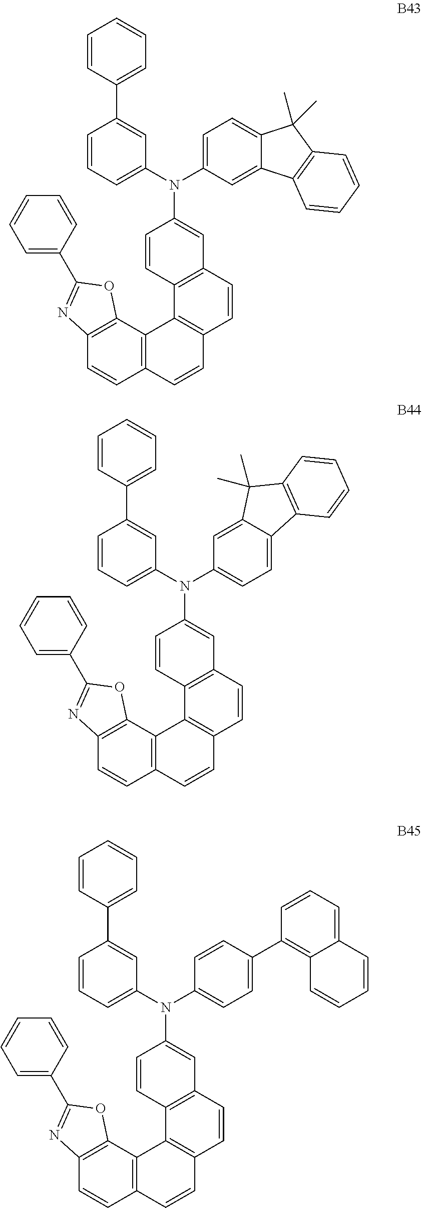

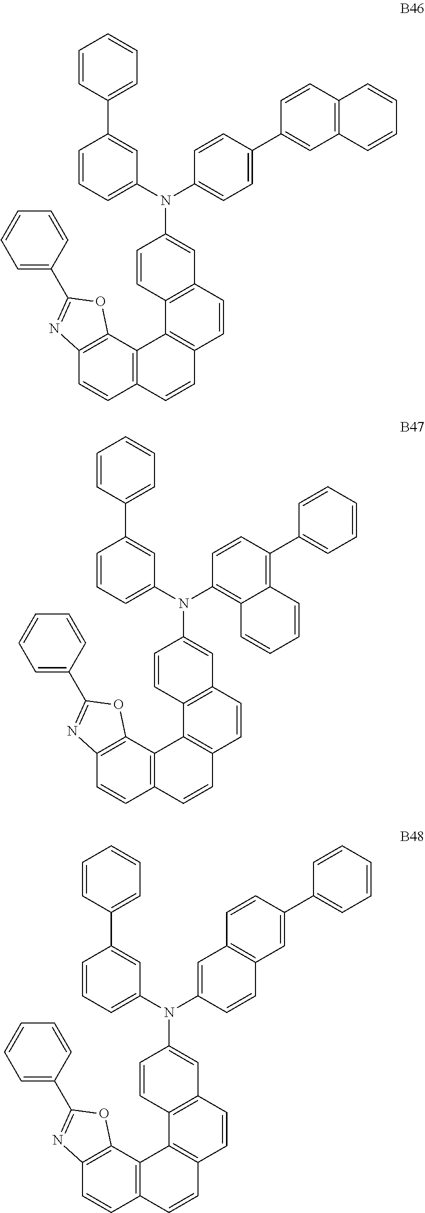

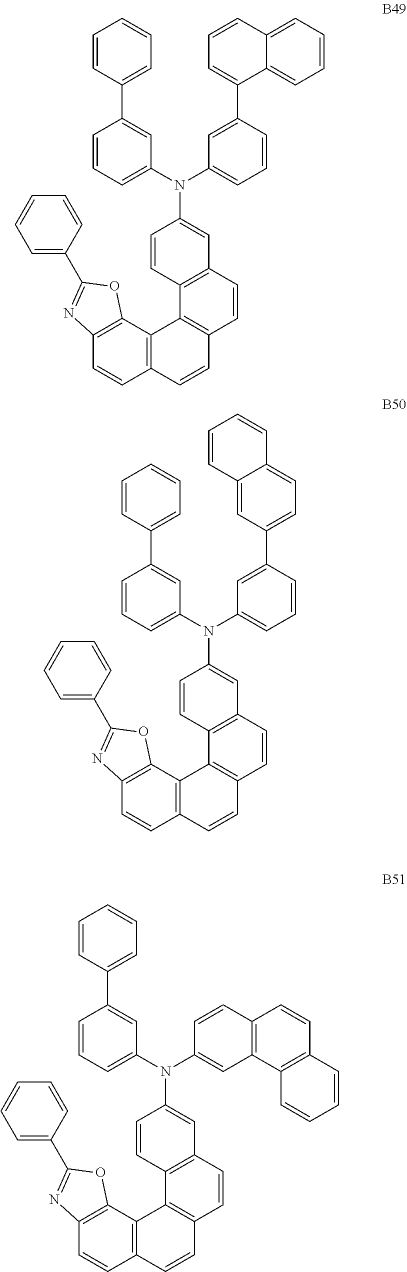 [SMILES: c1ccc(-c2cccc(N(c3cccc(-c4cccc5ccccc45)c3)c3ccc4c(ccc5ccc6ccc7nc(-c8ccccc8)oc7c6c54)c3)c2)cc1 ; c1ccc(-c2cccc(N(c3cccc(-c4ccc5ccccc5c4)c3)c3ccc4c(ccc5ccc6ccc7nc(-c8ccccc8)oc7c6c54)c3)c2)cc1 ; c1ccc(-c2cccc(N(c3ccc4c(ccc5ccc6ccc7nc(-c8ccccc8)oc7c6c54)c3)c3ccc4ccc5ccccc5c4c3)c2)cc1]

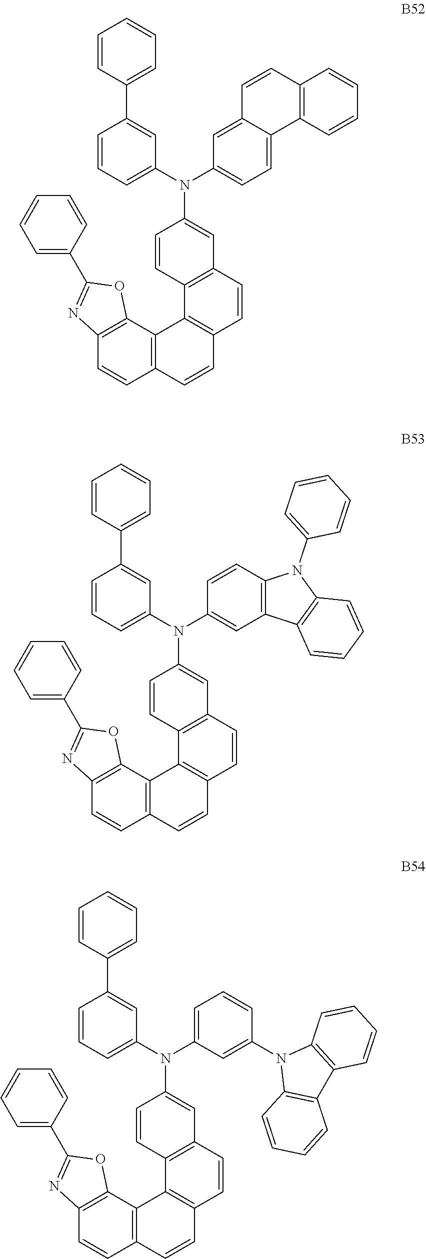 [SMILES: c1ccc(-c2cccc(N(c3ccc4c(ccc5ccccc54)c3)c3ccc4c(ccc5ccc6ccc7nc(-c8ccccc8)oc7c6c54)c3)c2)cc1 ; c1ccc(-c2cccc(N(c3ccc4c(ccc5ccc6ccc7nc(-c8ccccc8)oc7c6c54)c3)c3ccc4c(c3)c3ccccc3n4-c3ccccc3)c2)cc1 ; c1ccc(-c2cccc(N(c3cccc(-n4c5ccccc5c5ccccc54)c3)c3ccc4c(ccc5ccc6ccc7nc(-c8ccccc8)oc7c6c54)c3)c2)cc1]

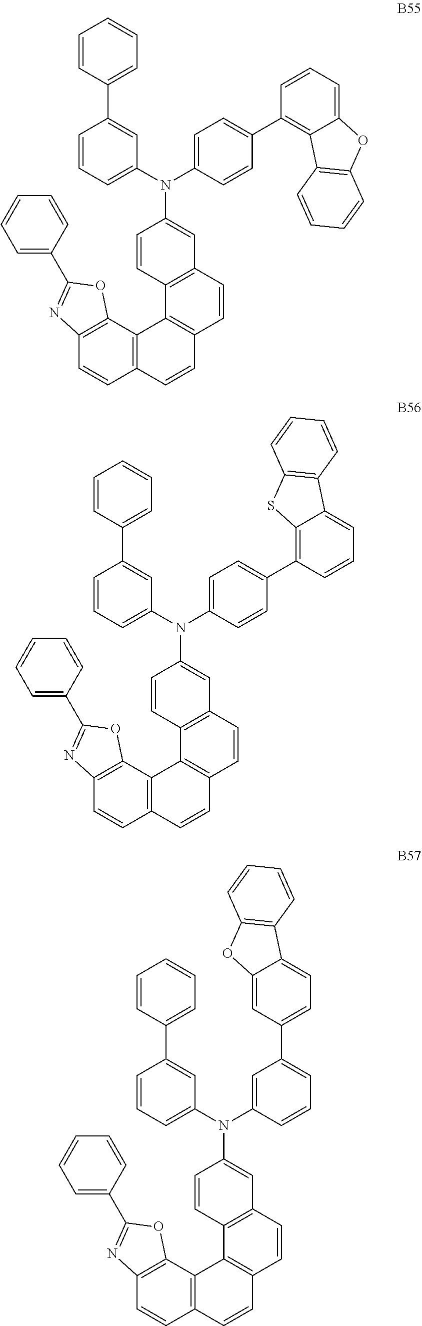 [SMILES: c1ccc(-c2cccc(N(c3ccc(-c4cccc5oc6ccccc6c45)cc3)c3ccc4c(ccc5ccc6ccc7nc(-c8ccccc8)oc7c6c54)c3)c2)cc1 ; c1ccc(-c2cccc(N(c3ccc(-c4cccc5c4sc4ccccc45)cc3)c3ccc4c(ccc5ccc6ccc7nc(-c8ccccc8)oc7c6c54)c3)c2)cc1 ; c1ccc(-c2cccc(N(c3cccc(-c4ccc5c(c4)oc4ccccc45)c3)c3ccc4c(ccc5ccc6ccc7nc(-c8ccccc8)oc7c6c54)c3)c2)cc1]

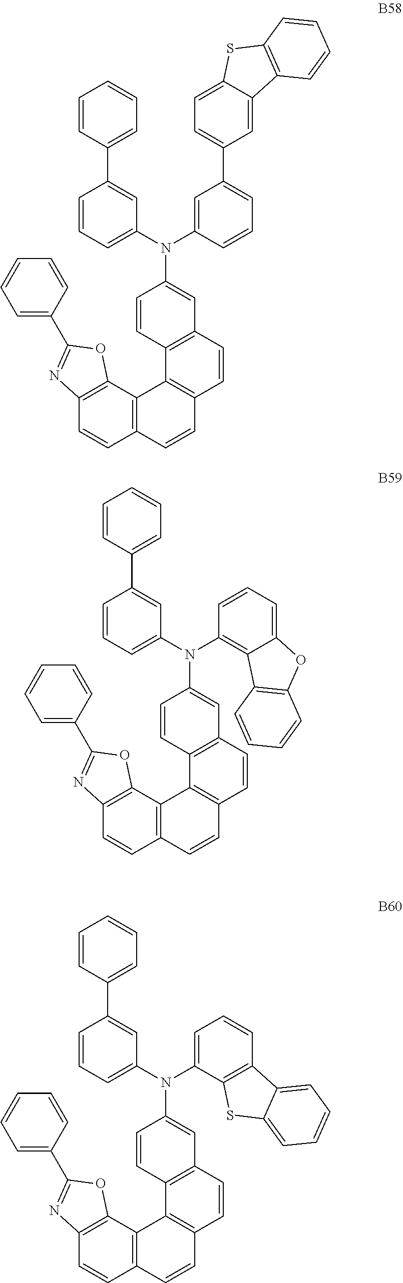

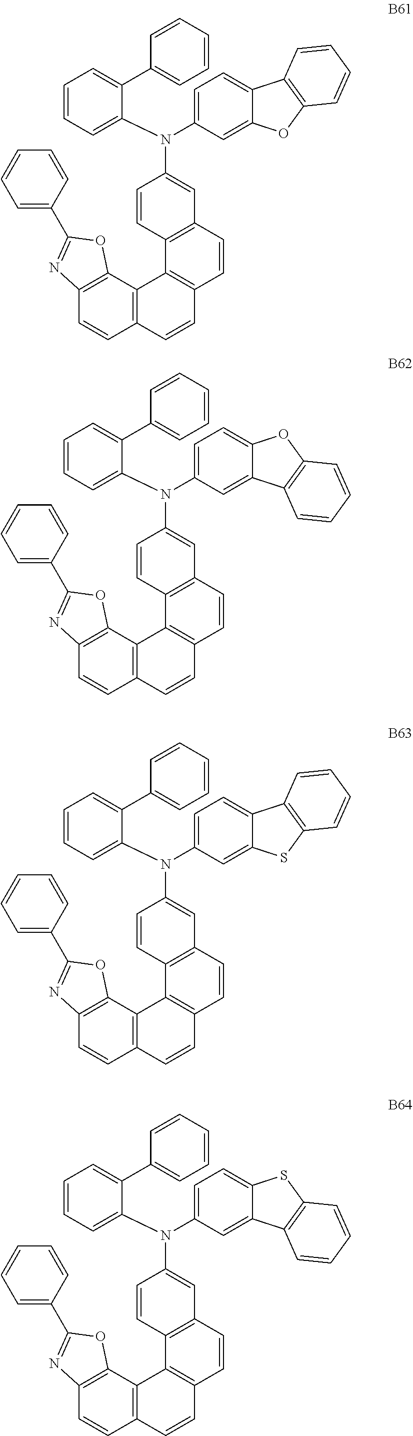

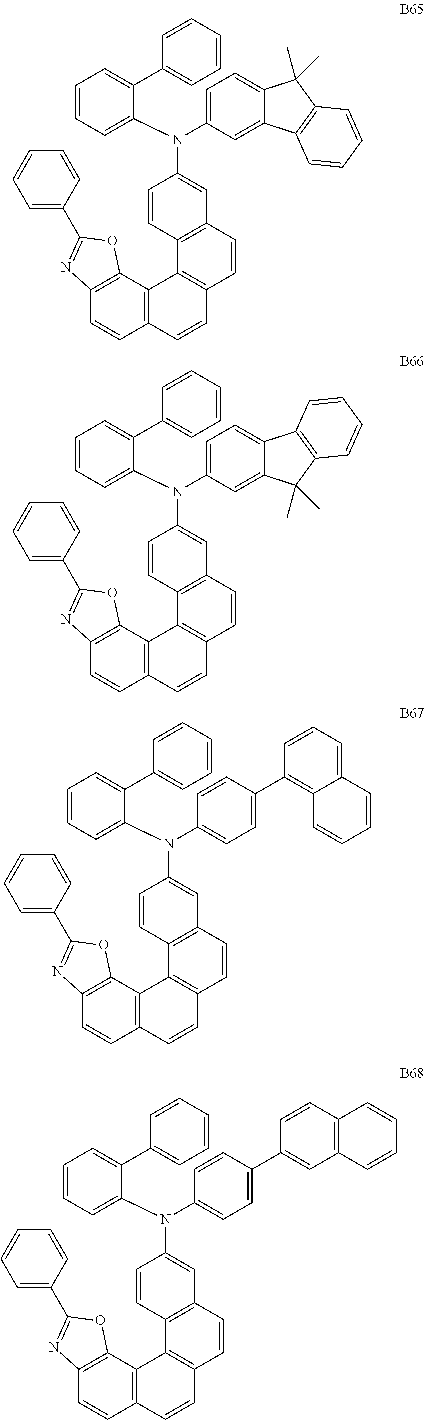

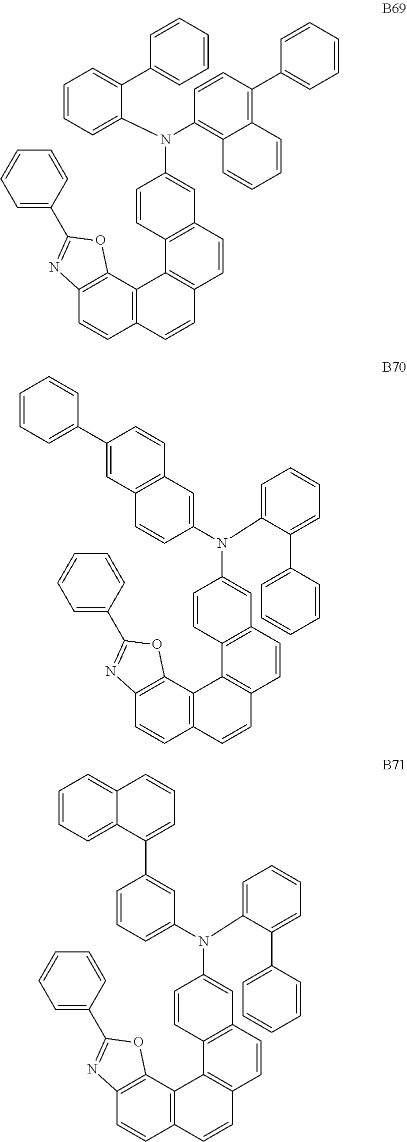

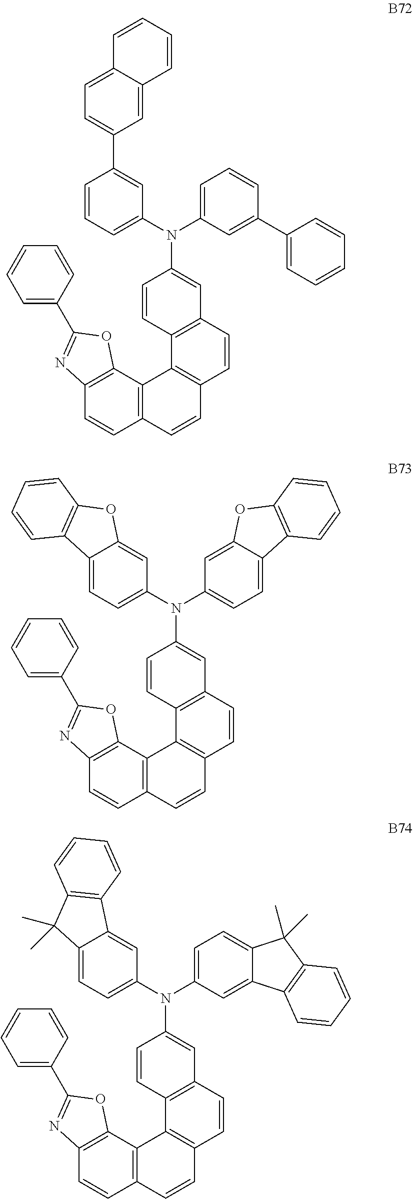

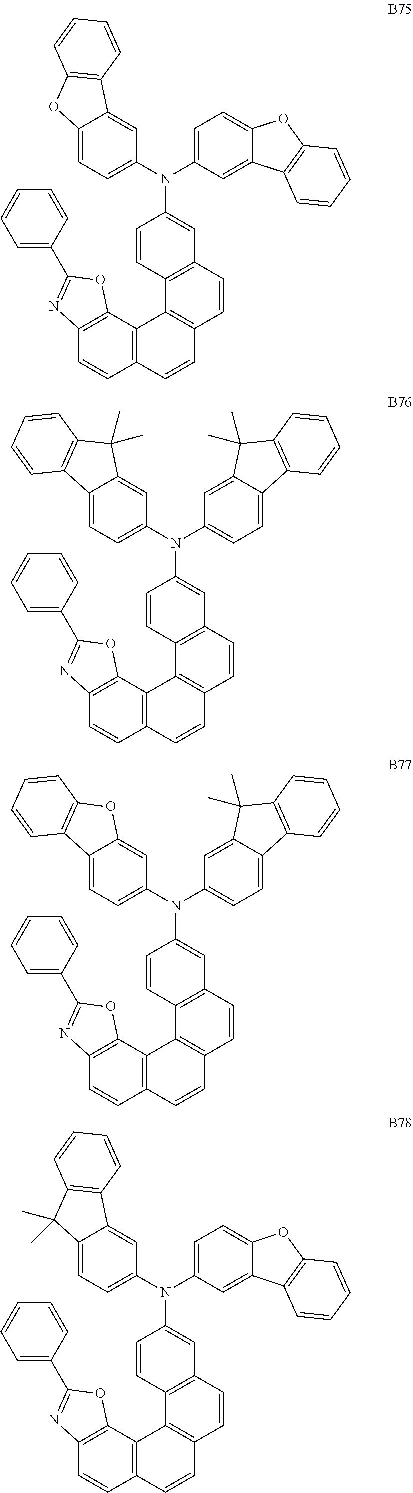

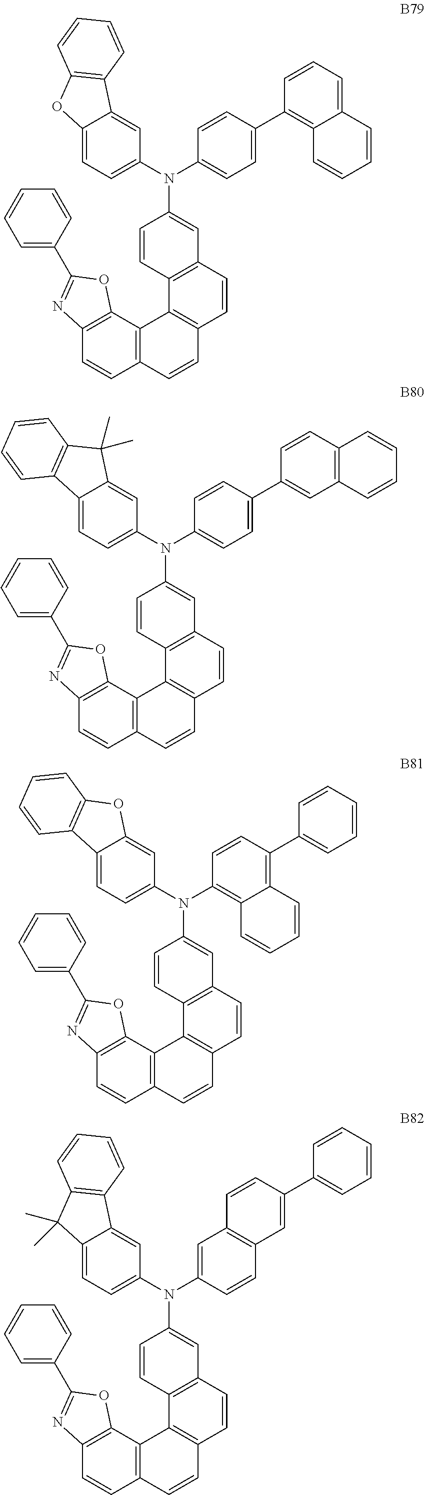

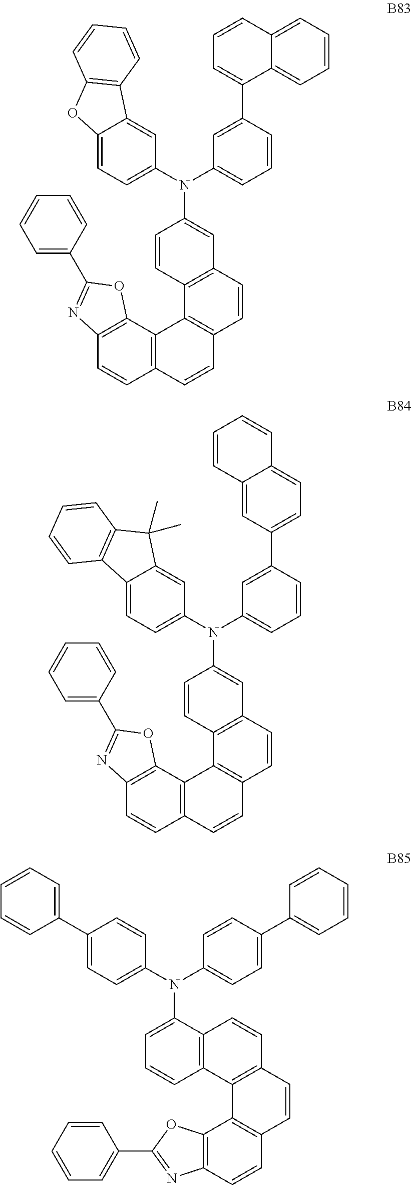 [SMILES: c1ccc(-c2nc3ccc4ccc5ccc6cc(N(c7cccc(-c8cccc9ccccc89)c7)c7ccc8oc9ccccc9c8c7)ccc6c5c4c3o2)cc1 ; CC1(C)c2ccccc2-c2ccc(N(c3cccc(-c4ccc5ccccc5c4)c3)c3ccc4c(ccc5ccc6ccc7nc(-c8ccccc8)oc7c6c54)c3)cc21 ; c1ccc(-c2ccc(N(c3ccc(-c4ccccc4)cc3)c3cccc4c3ccc3ccc5ccc6nc(-c7ccccc7)oc6c5c34)cc2)cc1]

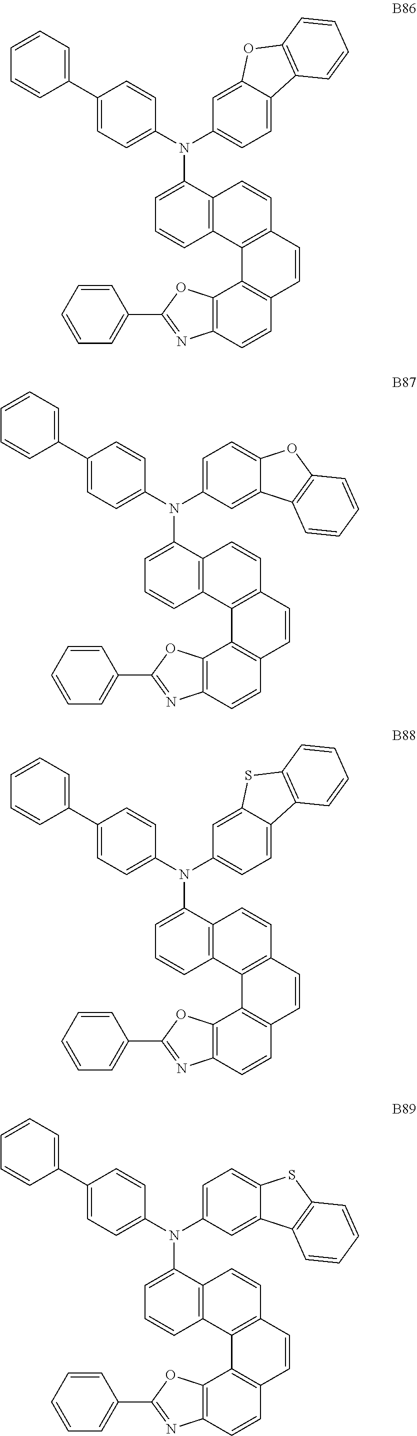 [SMILES: c1ccc(-c2ccc(N(c3ccc4c(c3)oc3ccccc34)c3cccc4c3ccc3ccc5ccc6nc(-c7ccccc7)oc6c5c34)cc2)cc1 ; c1ccc(-c2ccc(N(c3ccc4oc5ccccc5c4c3)c3cccc4c3ccc3ccc5ccc6nc(-c7ccccc7)oc6c5c34)cc2)cc1 ; c1ccc(-c2ccc(N(c3ccc4c(c3)sc3ccccc34)c3cccc4c3ccc3ccc5ccc6nc(-c7ccccc7)oc6c5c34)cc2)cc1 ; c1ccc(-c2ccc(N(c3ccc4sc5ccccc5c4c3)c3cccc4c3ccc3ccc5ccc6nc(-c7ccccc7)oc6c5c34)cc2)cc1]

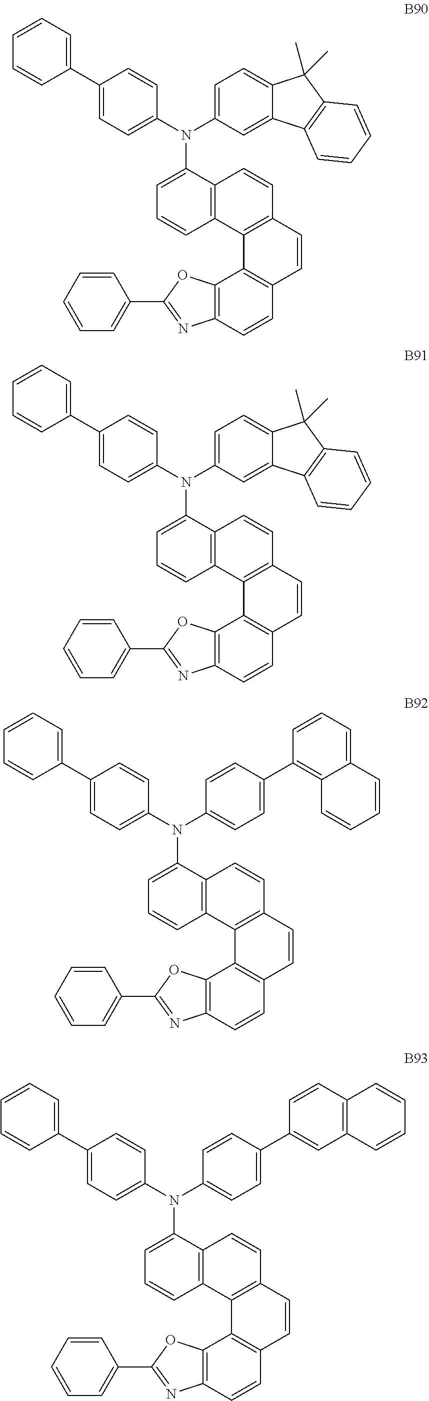 [SMILES: CC1(C)c2ccccc2-c2cc(N(c3ccc(-c4ccccc4)cc3)c3cccc4c3ccc3ccc5ccc6nc(-c7ccccc7)oc6c5c34)ccc21 ; CC1(C)c2ccccc2-c2cc(N(c3ccc(-c4ccccc4)cc3)c3cccc4c3ccc3ccc5ccc6nc(-c7ccccc7)oc6c5c34)ccc21 ; c1ccc(-c2ccc(N(c3ccc(-c4cccc5ccccc45)cc3)c3cccc4c3ccc3ccc5ccc6nc(-c7ccccc7)oc6c5c34)cc2)cc1 ; c1ccc(-c2ccc(N(c3ccc(-c4ccc5ccccc5c4)cc3)c3cccc4c3ccc3ccc5ccc6nc(-c7ccccc7)oc6c5c34)cc2)cc1]

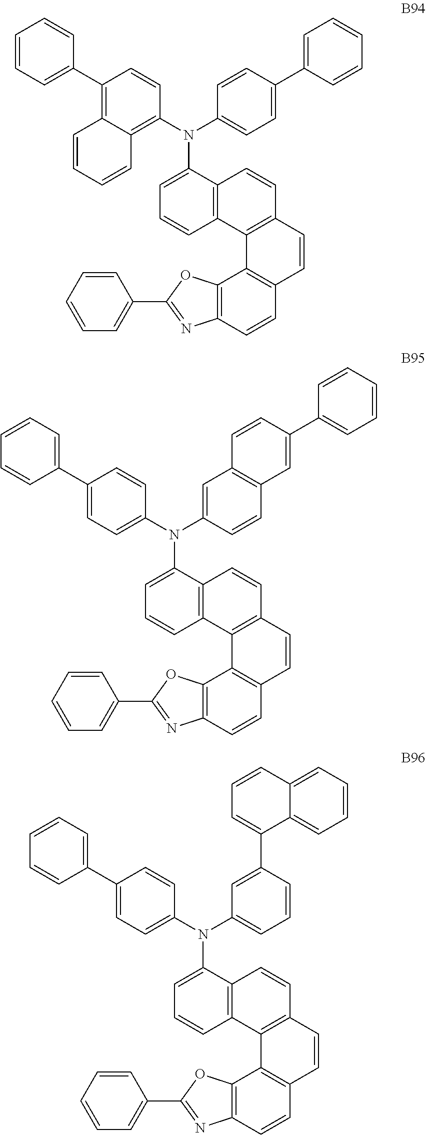

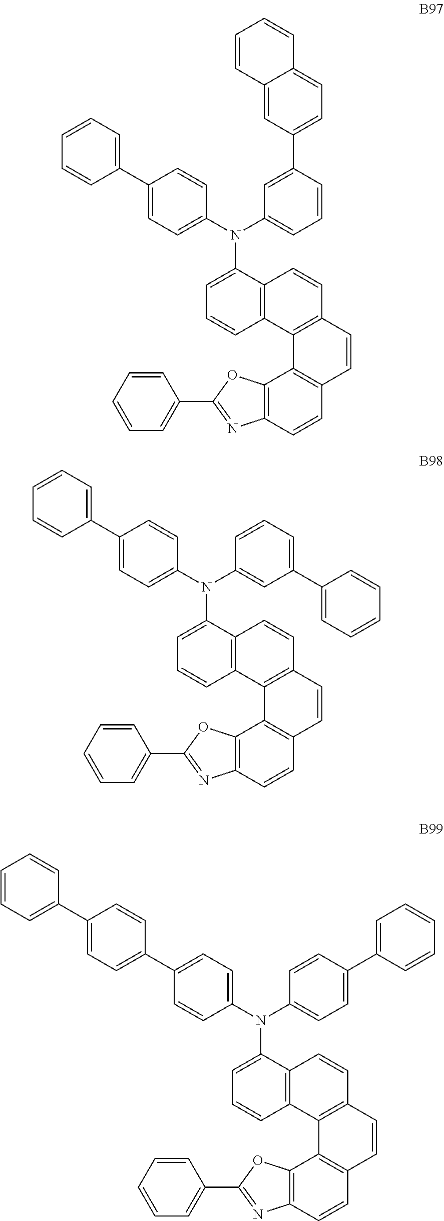 [SMILES: c1ccc(-c2ccc(N(c3cccc(-c4ccc5ccccc5c4)c3)c3cccc4c3ccc3ccc5ccc6nc(-c7ccccc7)oc6c5c34)cc2)cc1 ; c1ccc(-c2ccc(N(c3cccc(-c4ccccc4)c3)c3cccc4c3ccc3ccc5ccc6nc(-c7ccccc7)oc6c5c34)cc2)cc1 ; c1ccc(-c2ccc(-c3ccc(N(c4ccc(-c5ccccc5)cc4)c4cccc5c4ccc4ccc6ccc7nc(-c8ccccc8)oc7c6c45)cc3)cc2)cc1]

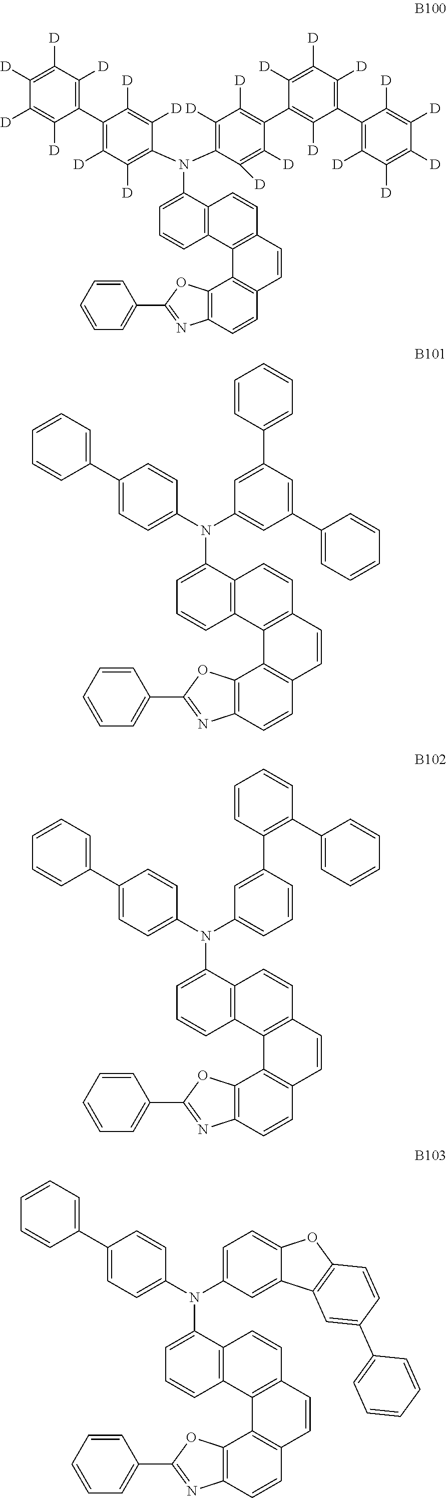

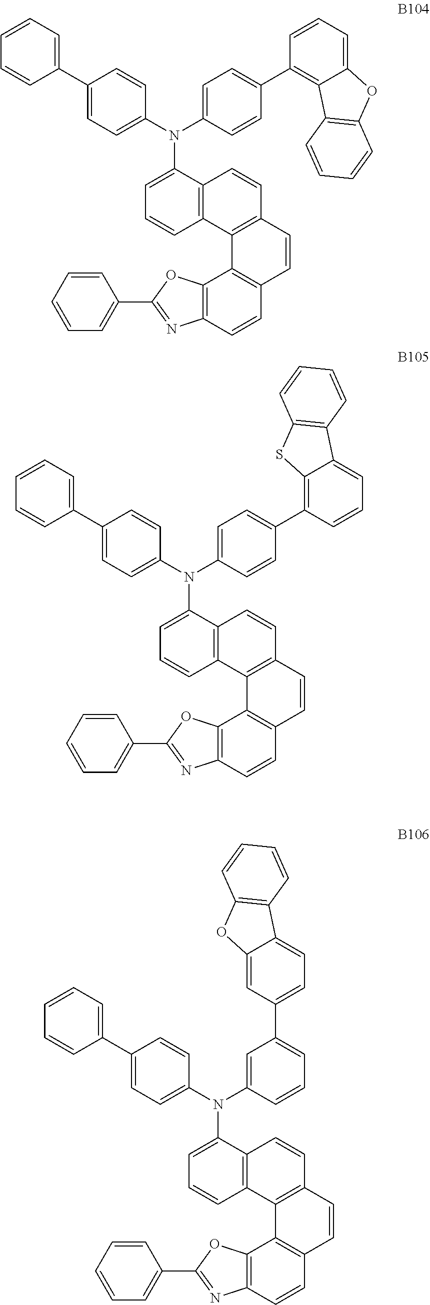 [SMILES: c1ccc(-c2ccc(N(c3ccc(-c4cccc5oc6ccccc6c45)cc3)c3cccc4c3ccc3ccc5ccc6nc(-c7ccccc7)oc6c5c34)cc2)cc1 ; c1ccc(-c2ccc(N(c3ccc(-c4cccc5c4sc4ccccc45)cc3)c3cccc4c3ccc3ccc5ccc6nc(-c7ccccc7)oc6c5c34)cc2)cc1 ; c1ccc(-c2ccc(N(c3cccc(-c4ccc5c(c4)oc4ccccc45)c3)c3cccc4c3ccc3ccc5ccc6nc(-c7ccccc7)oc6c5c34)cc2)cc1]

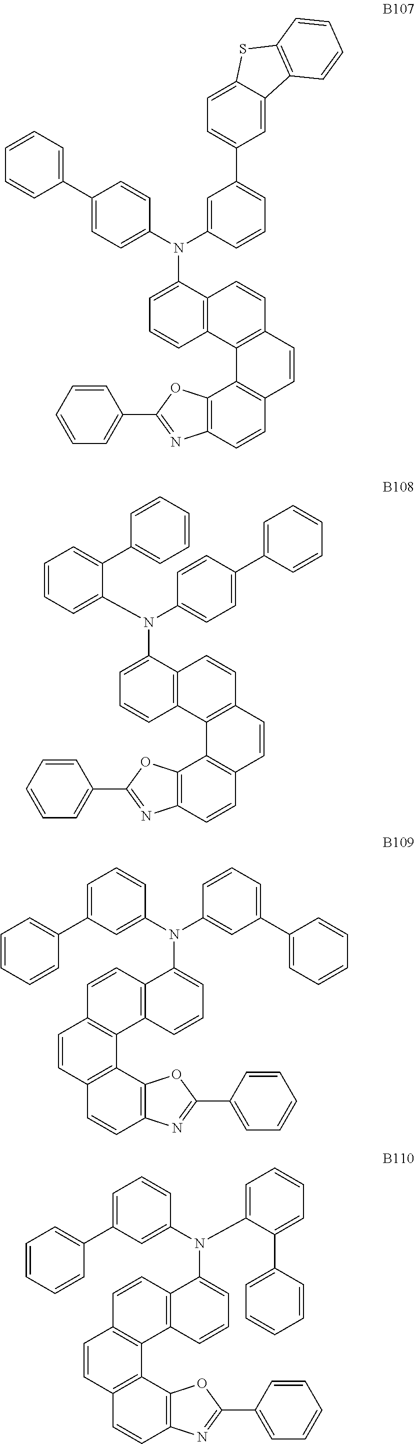

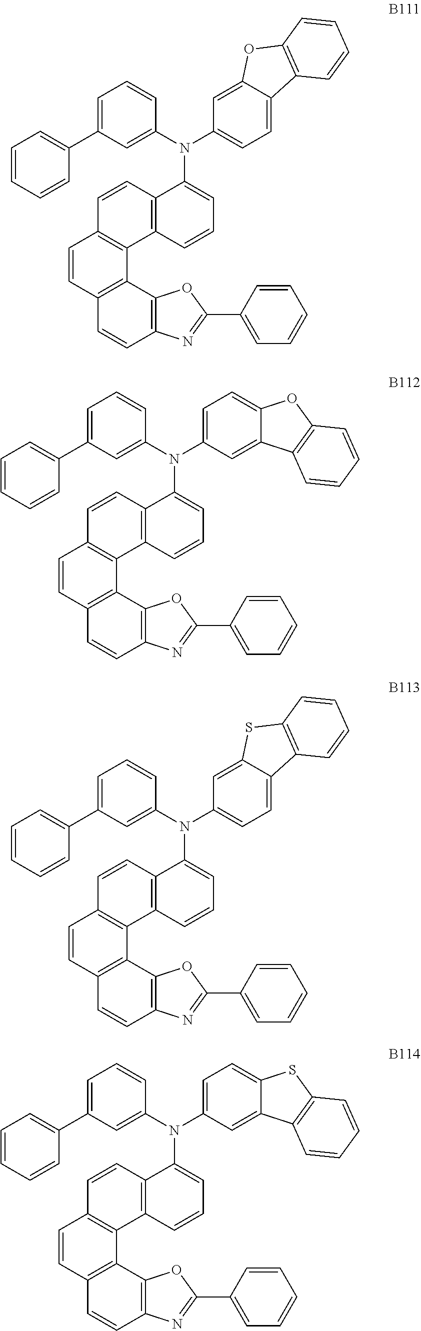

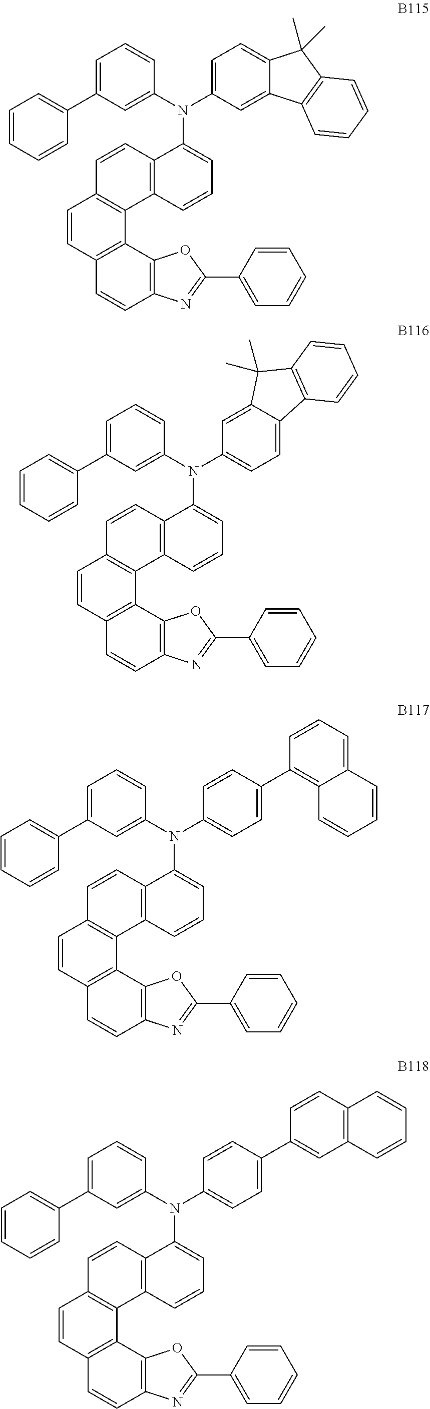

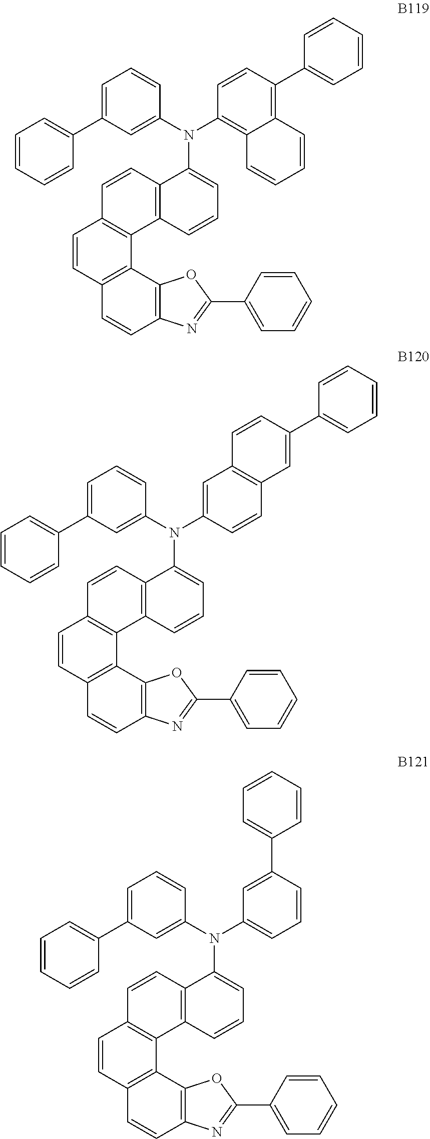

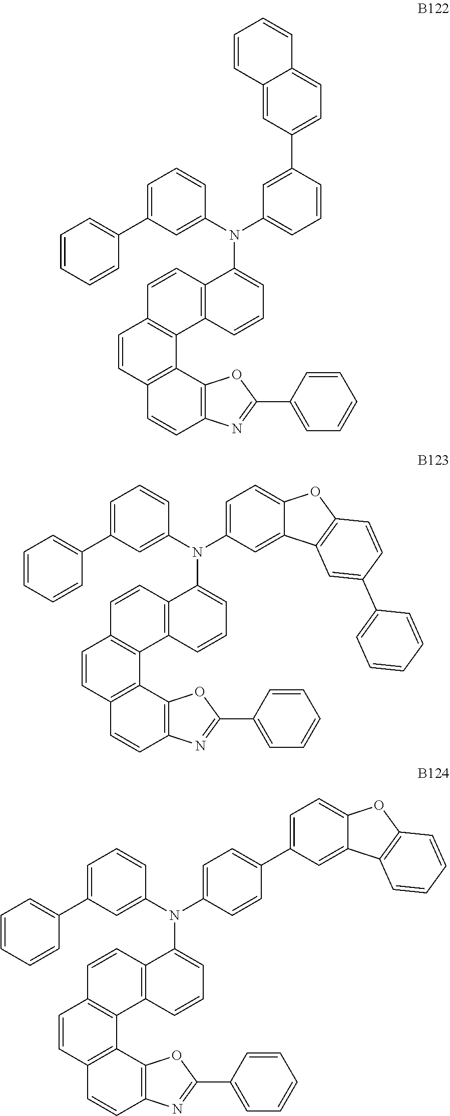 [SMILES: c1ccc(-c2cccc(N(c3cccc(-c4ccc5ccccc5c4)c3)c3cccc4c3ccc3ccc5ccc6nc(-c7ccccc7)oc6c5c34)c2)cc1 ; c1ccc(-c2cccc(N(c3ccc4oc5ccc(-c6ccccc6)cc5c4c3)c3cccc4c3ccc3ccc5ccc6nc(-c7ccccc7)oc6c5c34)c2)cc1 ; c1ccc(-c2cccc(N(c3ccc(-c4ccc5oc6ccccc6c5c4)cc3)c3cccc4c3ccc3ccc5ccc6nc(-c7ccccc7)oc6c5c34)c2)cc1]

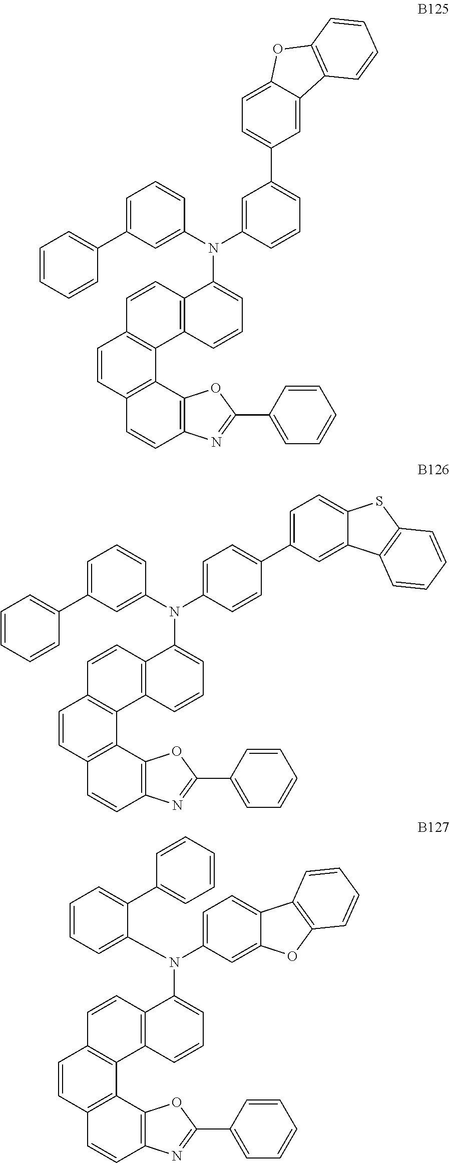

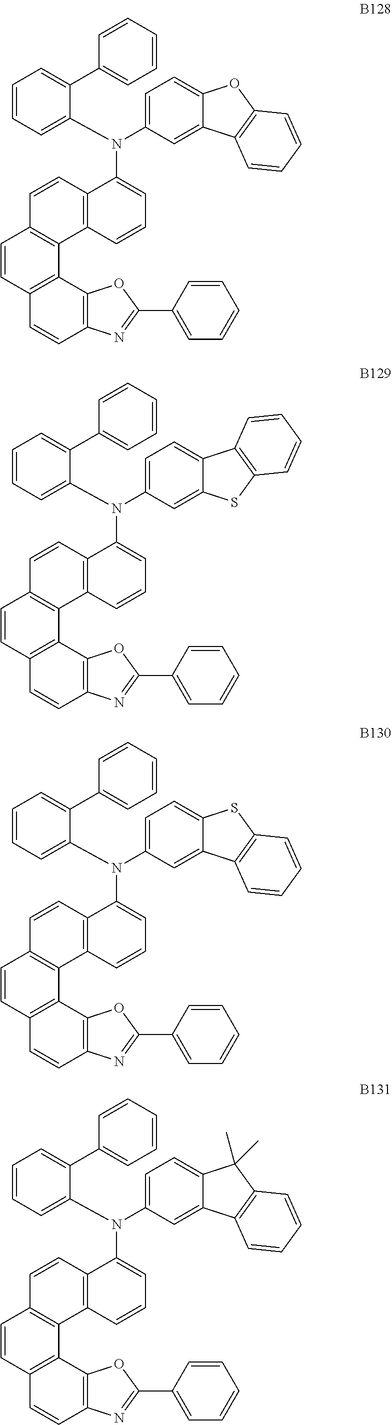

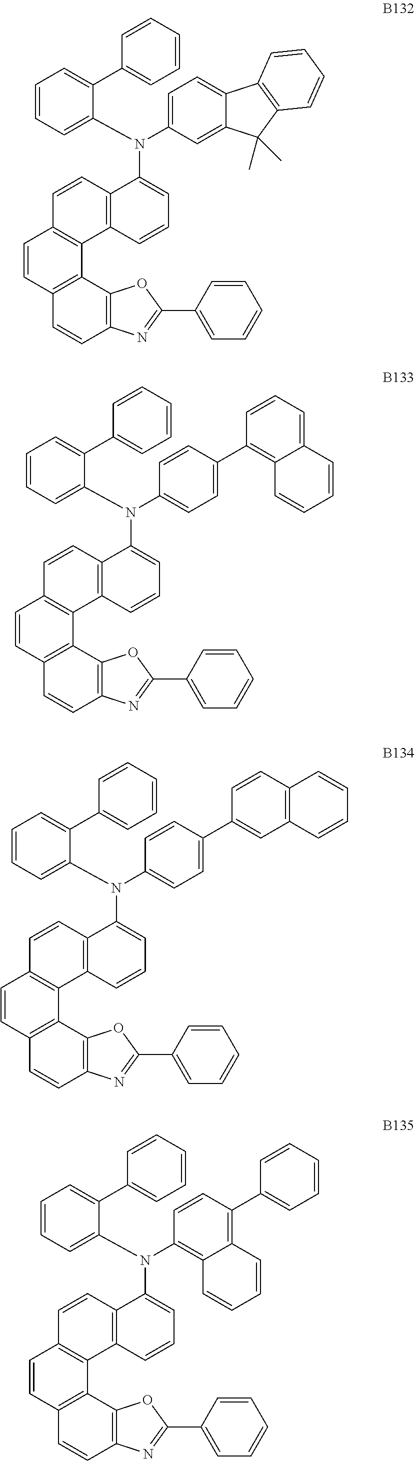 [SMILES: CC1(C)c2ccccc2-c2ccc(N(c3ccccc3-c3ccccc3)c3cccc4c3ccc3ccc5ccc6nc(-c7ccccc7)oc6c5c34)cc21 ; c1ccc(-c2nc3ccc4ccc5ccc6c(N(c7ccc(-c8cccc9ccccc89)cc7)c7ccccc7-c7ccccc7)cccc6c5c4c3o2)cc1 ; c1ccc(-c2nc3ccc4ccc5ccc6c(N(c7ccc(-c8ccc9ccccc9c8)cc7)c7ccccc7-c7ccccc7)cccc6c5c4c3o2)cc1 ; c1ccc(-c2nc3ccc4ccc5ccc6c(N(c7ccccc7-c7ccccc7)c7ccc(-c8ccccc8)c8ccccc78)cccc6c5c4c3o2)cc1]

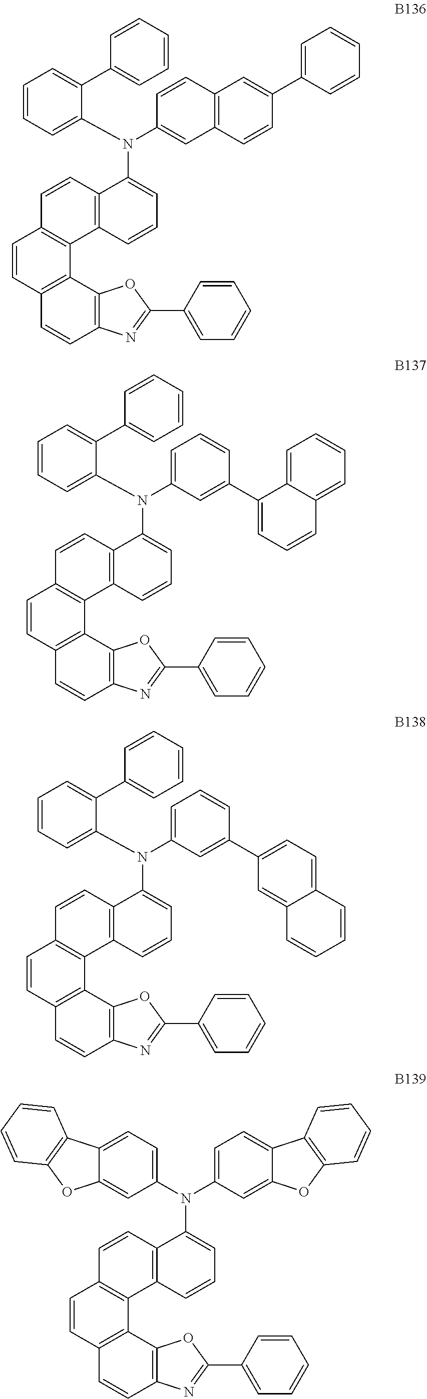

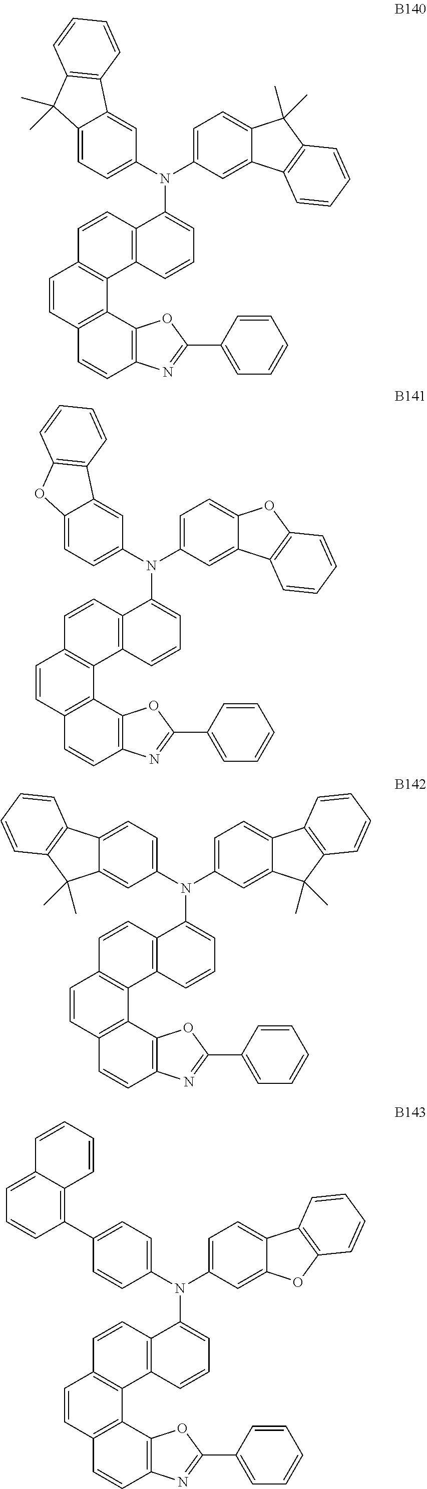

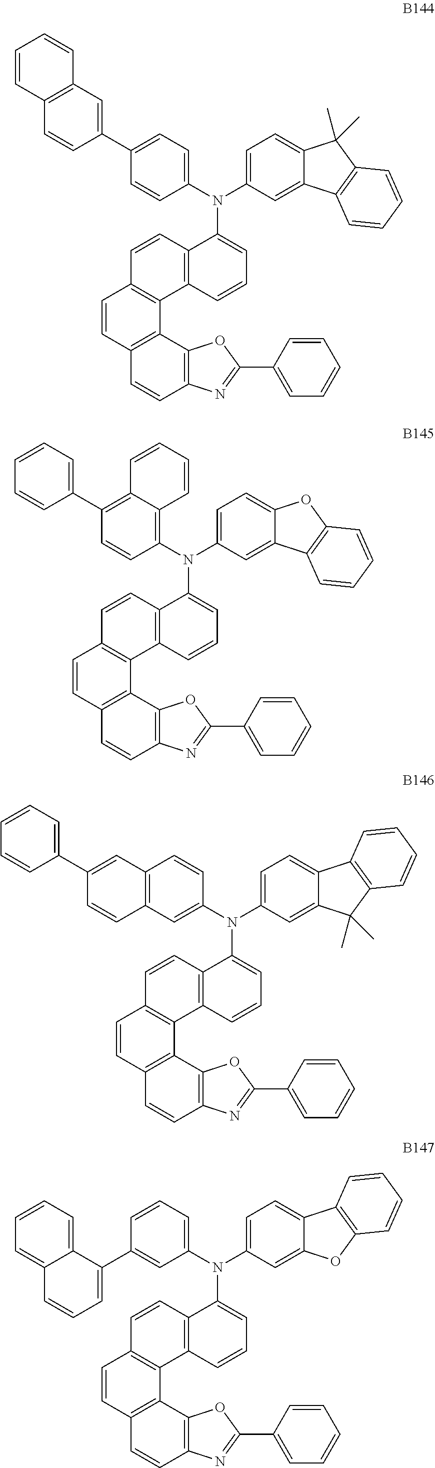

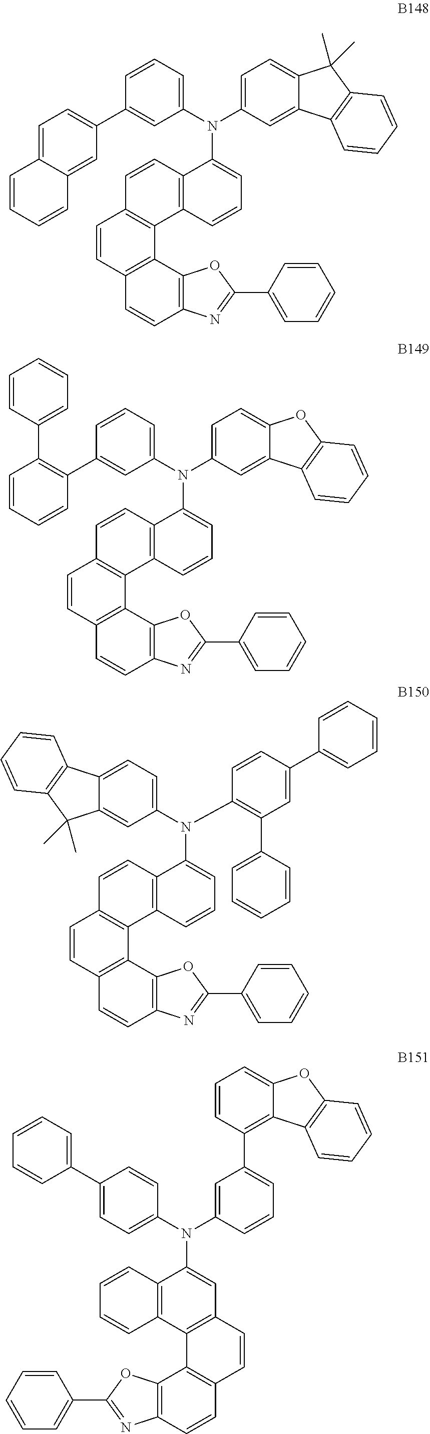

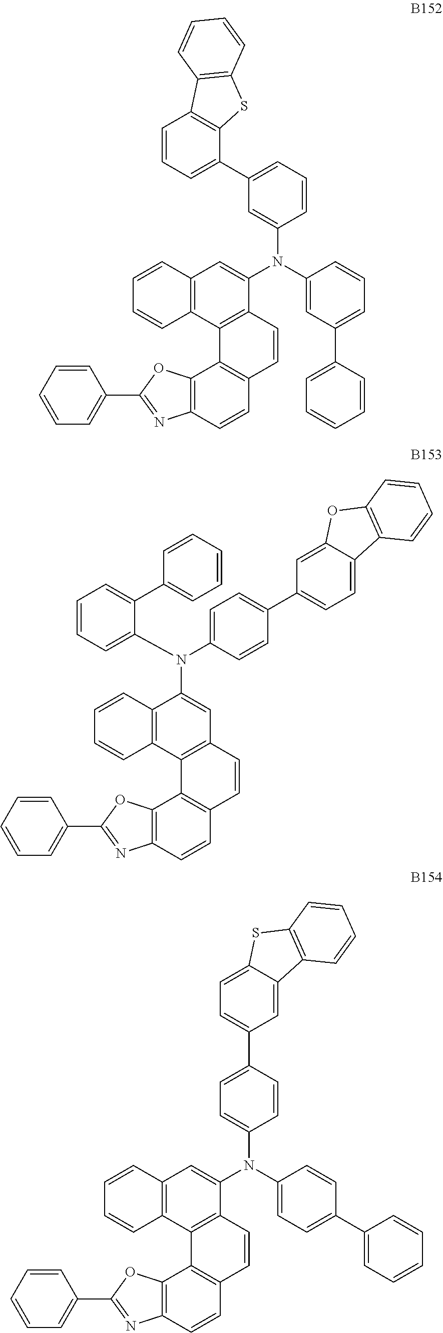 [SMILES: c1ccc(-c2cccc(N(c3cccc(-c4cccc5c4sc4ccccc45)c3)c3cc4ccccc4c4c3ccc3ccc5nc(-c6ccccc6)oc5c34)c2)cc1 ; c1ccc(-c2nc3ccc4ccc5cc(N(c6ccc(-c7ccc8c(c7)oc7ccccc78)cc6)c6ccccc6-c6ccccc6)c6ccccc6c5c4c3o2)cc1 ; c1ccc(-c2ccc(N(c3ccc(-c4ccc5sc6ccccc6c5c4)cc3)c3cc4ccccc4c4c3ccc3ccc5nc(-c6ccccc6)oc5c34)cc2)cc1]

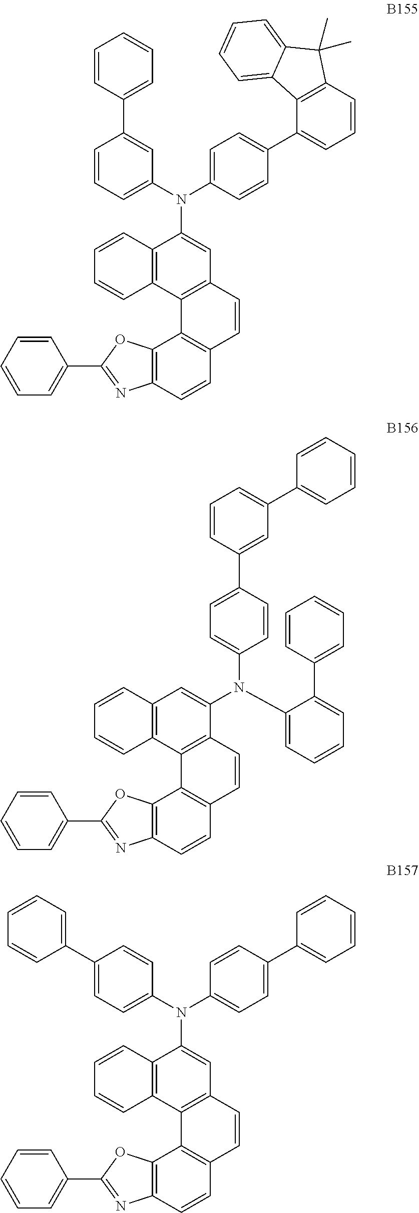 [SMILES: CC1(C)c2ccccc2-c2c(-c3ccc(N(c4cccc(-c5ccccc5)c4)c4cc5ccc6ccc7nc(-c8ccccc8)oc7c6c5c5ccccc45)cc3)cccc21 ; c1ccc(-c2cccc(-c3ccc(N(c4ccccc4-c4ccccc4)c4cc5ccccc5c5c4ccc4ccc6nc(-c7ccccc7)oc6c45)cc3)c2)cc1 ; c1ccc(-c2ccc(N(c3ccc(-c4ccccc4)cc3)c3cc4ccc5ccc6nc(-c7ccccc7)oc6c5c4c4ccccc34)cc2)cc1]

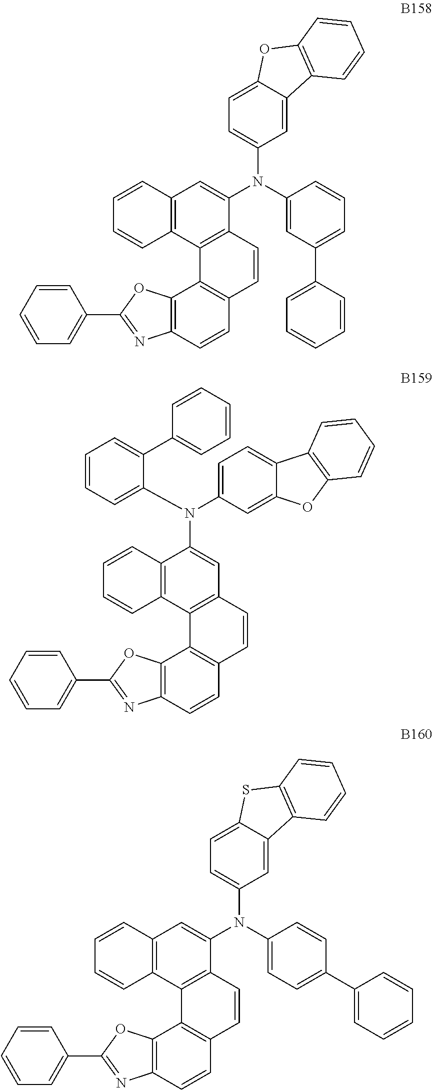

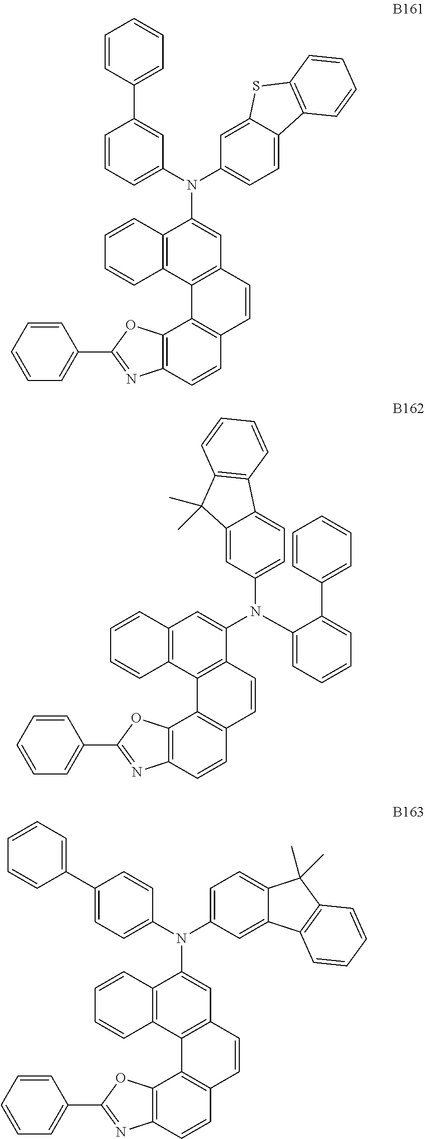

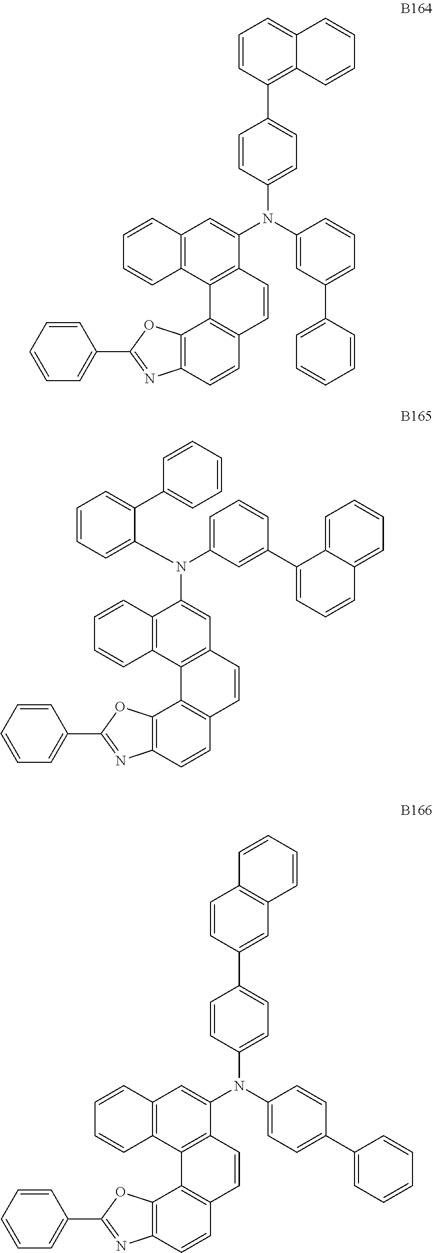 [SMILES: c1ccc(-c2cccc(N(c3ccc(-c4cccc5ccccc45)cc3)c3cc4ccccc4c4c3ccc3ccc5nc(-c6ccccc6)oc5c34)c2)cc1 ; c1ccc(-c2nc3ccc4ccc5cc(N(c6cccc(-c7cccc8ccccc78)c6)c6ccccc6-c6ccccc6)c6ccccc6c5c4c3o2)cc1 ; c1ccc(-c2ccc(N(c3ccc(-c4ccc5ccccc5c4)cc3)c3cc4ccccc4c4c3ccc3ccc5nc(-c6ccccc6)oc5c34)cc2)cc1]

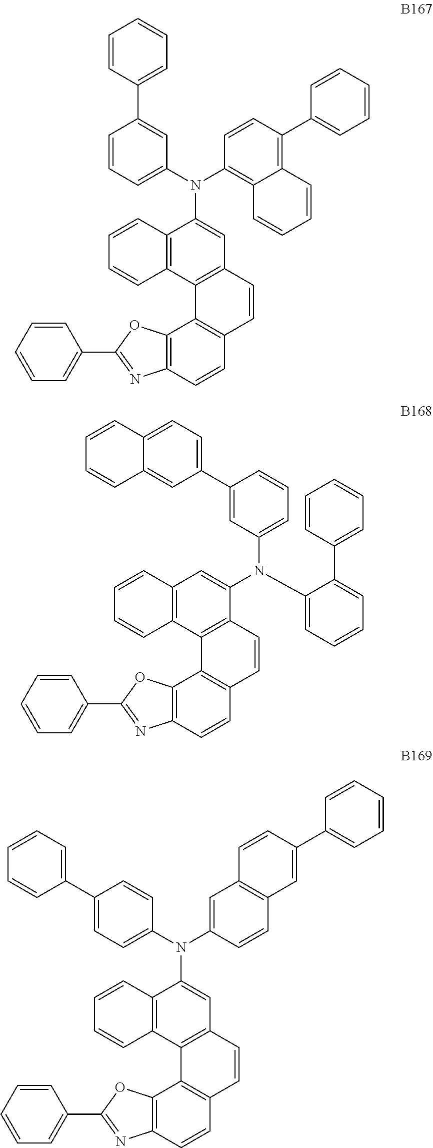

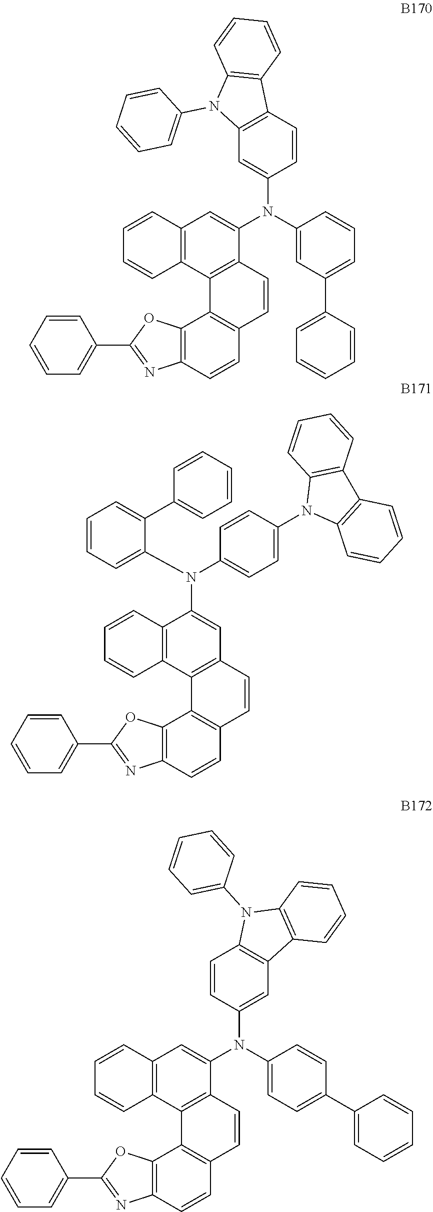

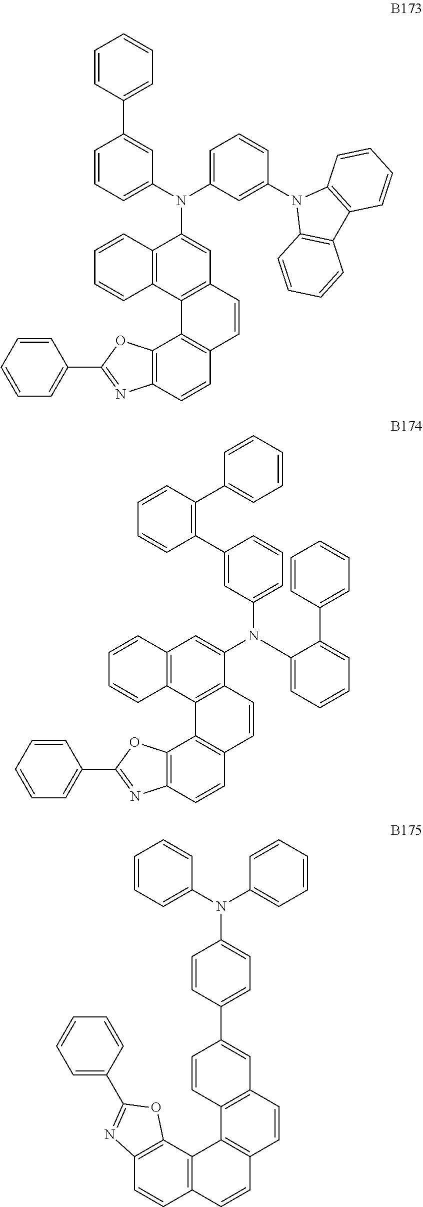 [SMILES: c1ccc(-c2cccc(N(c3cccc(-n4c5ccccc5c5ccccc54)c3)c3cc4ccc5ccc6nc(-c7ccccc7)oc6c5c4c4ccccc34)c2)cc1 ; c1ccc(-c2nc3ccc4ccc5c(N(c6cccc(-c7ccccc7-c7ccccc7)c6)c6ccccc6-c6ccccc6)cc6ccccc6c5c4c3o2)cc1 ; c1ccc(-c2nc3ccc4ccc5ccc6cc(-c7ccc(N(c8ccccc8)c8ccccc8)cc7)ccc6c5c4c3o2)cc1]

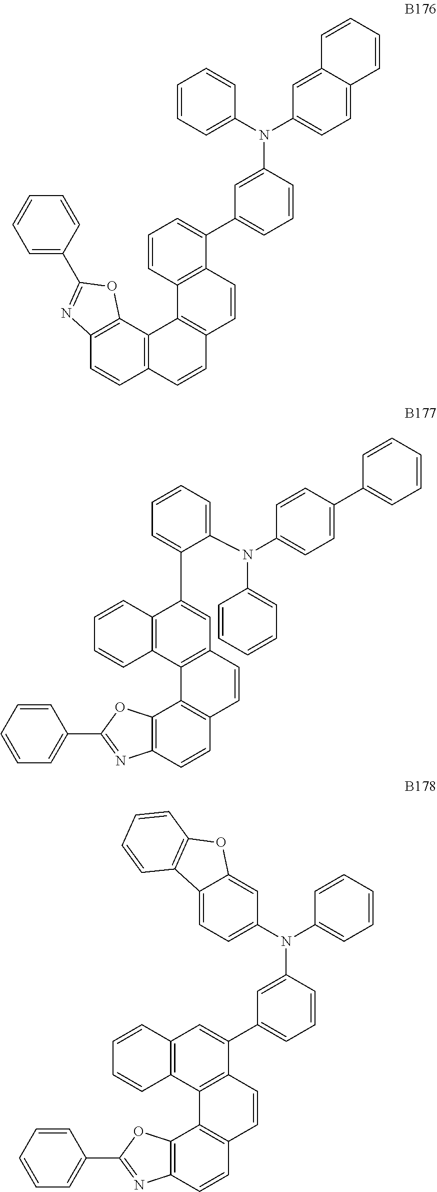

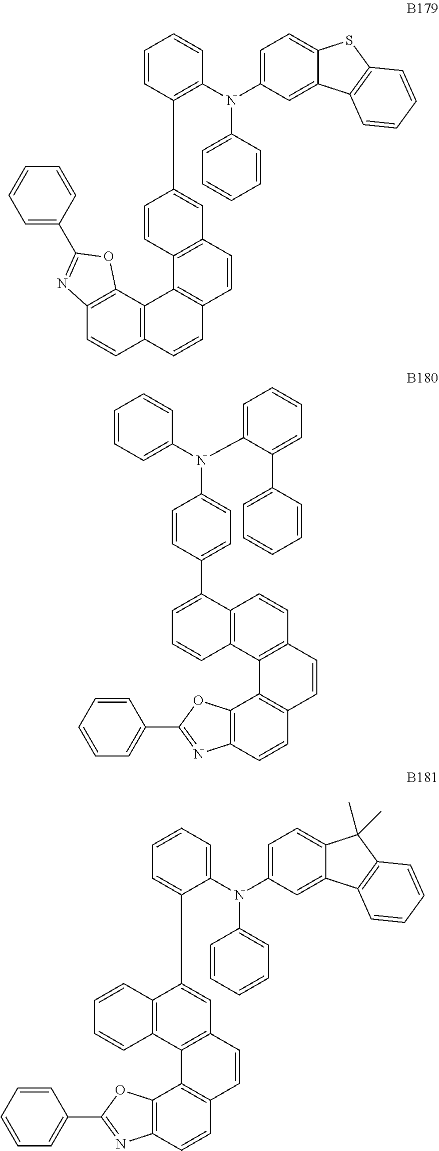 [SMILES: c1ccc(-c2nc3ccc4ccc5ccc6cc(-c7ccccc7N(c7ccccc7)c7ccc8sc9ccccc9c8c7)ccc6c5c4c3o2)cc1 ; c1ccc(-c2nc3ccc4ccc5ccc6c(-c7ccc(N(c8ccccc8)c8ccccc8-c8ccccc8)cc7)cccc6c5c4c3o2)cc1 ; CC1(C)c2ccccc2-c2cc(N(c3ccccc3)c3ccccc3-c3cc4ccc5ccc6nc(-c7ccccc7)oc6c5c4c4ccccc34)ccc21]

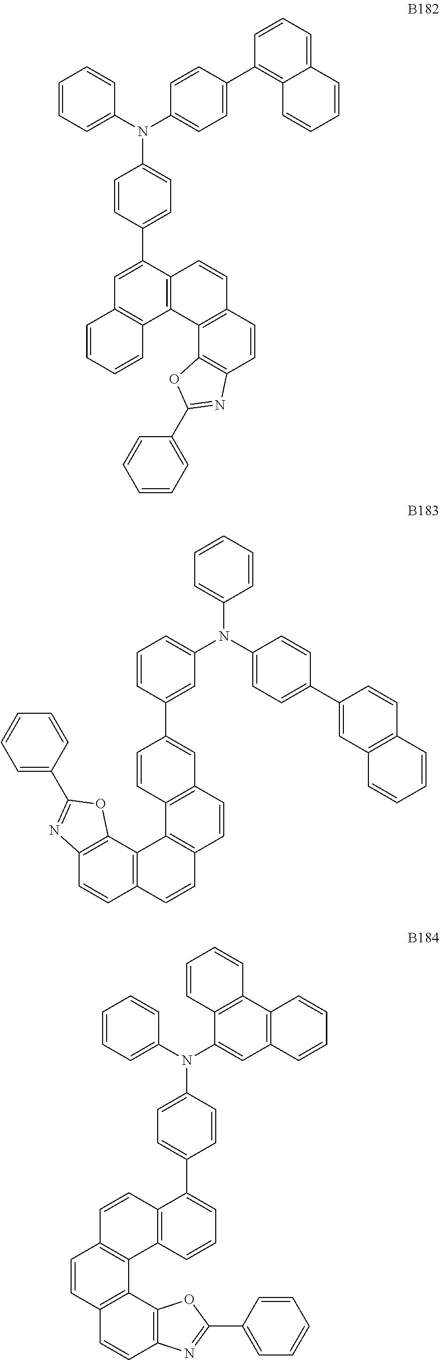 [SMILES: c1ccc(-c2nc3ccc4ccc5c(-c6ccc(N(c7ccccc7)c7ccc(-c8cccc9ccccc89)cc7)cc6)cc6ccccc6c5c4c3o2)cc1 ; c1ccc(-c2nc3ccc4ccc5ccc6cc(-c7cccc(N(c8ccccc8)c8ccc(-c9ccc%10ccccc%10c9)cc8)c7)ccc6c5c4c3o2)cc1 ; c1ccc(-c2nc3ccc4ccc5ccc6c(-c7ccc(N(c8ccccc8)c8cc9ccccc9c9ccccc89)cc7)cccc6c5c4c3o2)cc1]

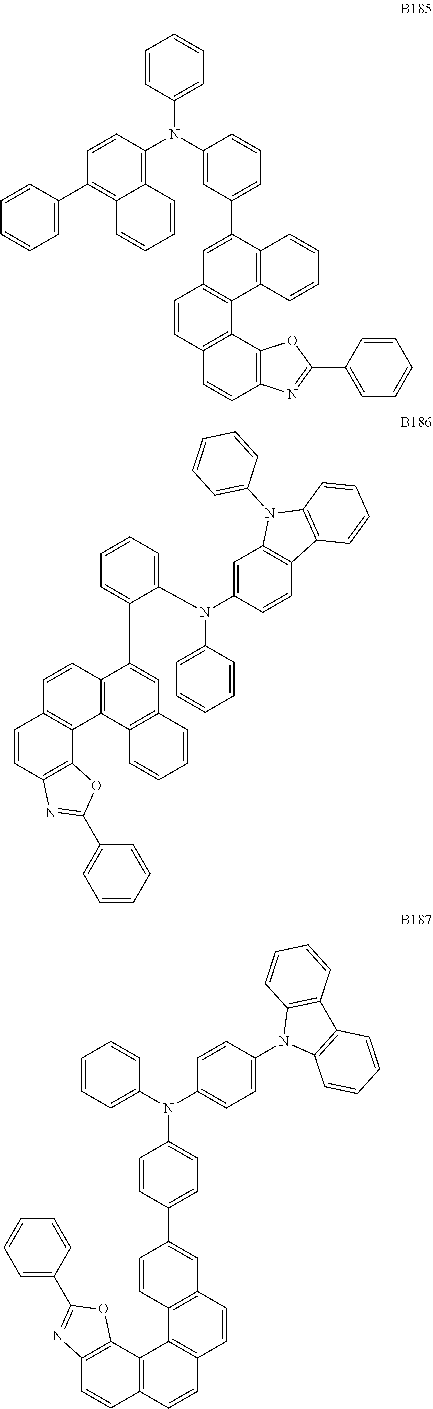

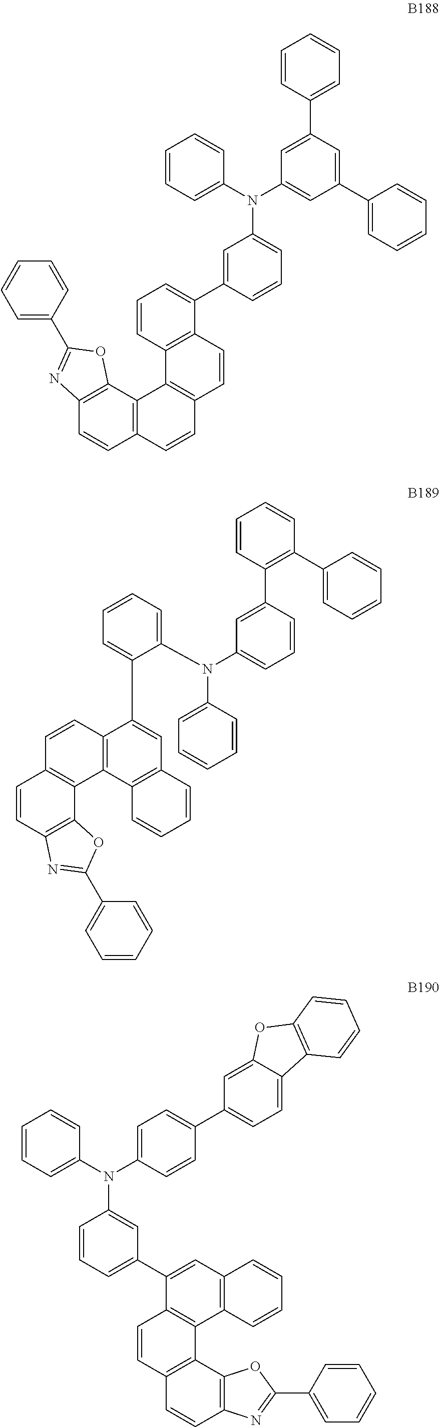 [SMILES: c1ccc(-c2cc(-c3ccccc3)cc(N(c3ccccc3)c3cccc(-c4cccc5c4ccc4ccc6ccc7nc(-c8ccccc8)oc7c6c45)c3)c2)cc1 ; c1ccc(-c2nc3ccc4ccc5c(-c6ccccc6N(c6ccccc6)c6cccc(-c7ccccc7-c7ccccc7)c6)cc6ccccc6c5c4c3o2)cc1 ; c1ccc(-c2nc3ccc4ccc5c(-c6cccc(N(c7ccccc7)c7ccc(-c8ccc9c(c8)oc8ccccc89)cc7)c6)cc6ccccc6c5c4c3o2)cc1]

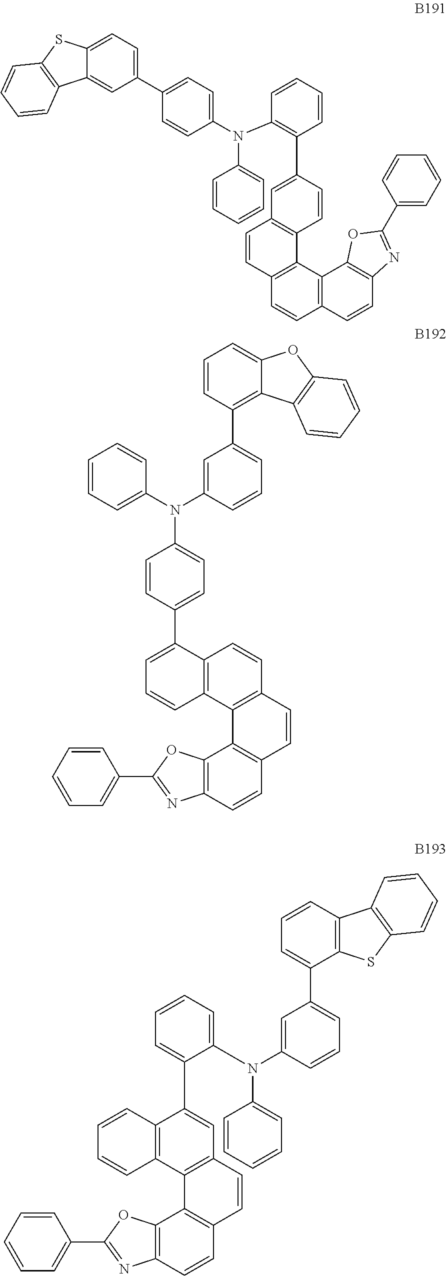

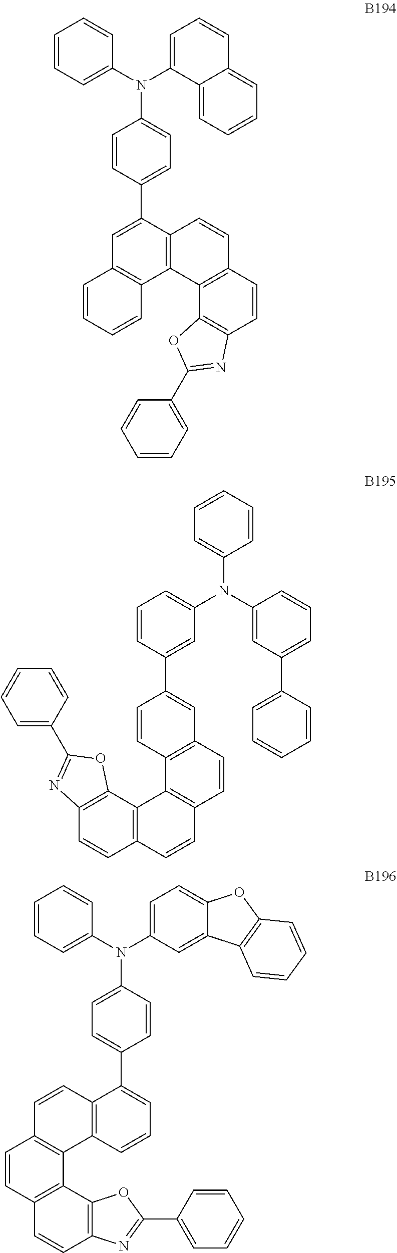

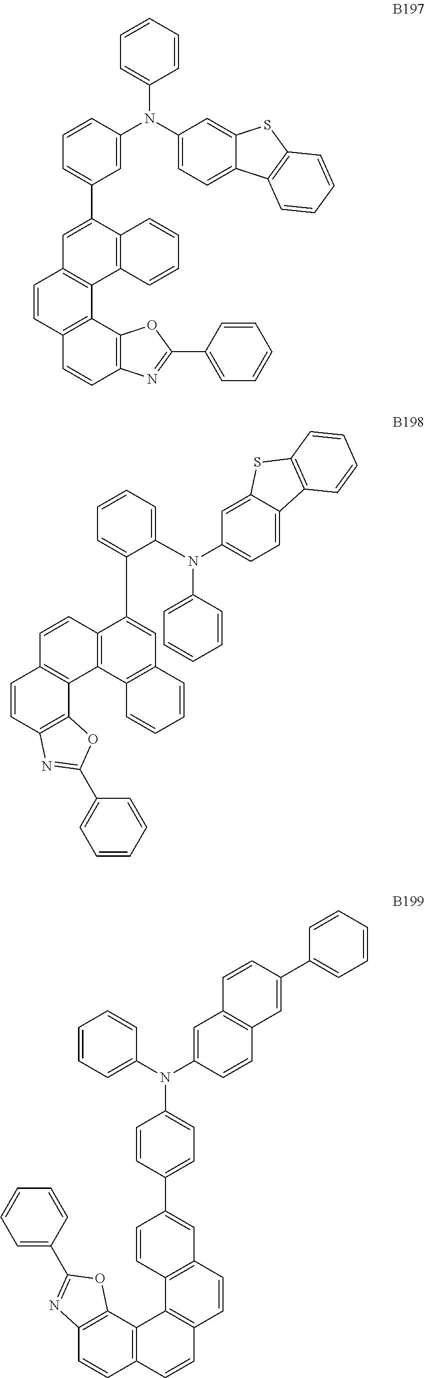 [SMILES: c1ccc(-c2nc3ccc4ccc5cc(-c6cccc(N(c7ccccc7)c7ccc8c(c7)sc7ccccc78)c6)c6ccccc6c5c4c3o2)cc1 ; c1ccc(-c2nc3ccc4ccc5c(-c6ccccc6N(c6ccccc6)c6ccc7c(c6)sc6ccccc67)cc6ccccc6c5c4c3o2)cc1 ; c1ccc(-c2ccc3cc(N(c4ccccc4)c4ccc(-c5ccc6c(ccc7ccc8ccc9nc(-c%10ccccc%10)oc9c8c76)c5)cc4)ccc3c2)cc1]

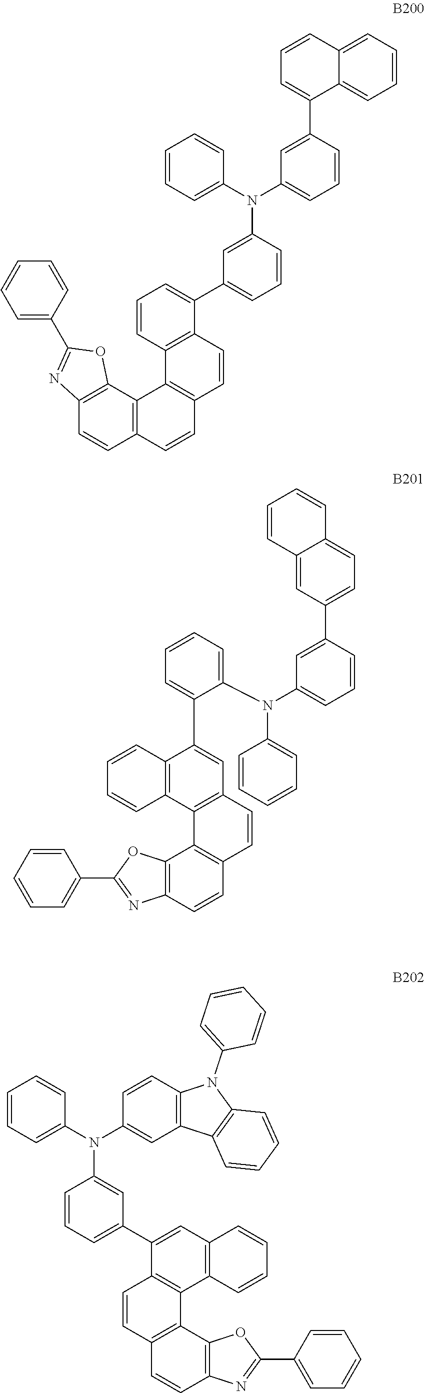 [SMILES: c1ccc(-c2nc3ccc4ccc5ccc6c(-c7cccc(N(c8ccccc8)c8cccc(-c9cccc%10ccccc9%10)c8)c7)cccc6c5c4c3o2)cc1 ; c1ccc(-c2nc3ccc4ccc5cc(-c6ccccc6N(c6ccccc6)c6cccc(-c7ccc8ccccc8c7)c6)c6ccccc6c5c4c3o2)cc1 ; c1ccc(-c2nc3ccc4ccc5c(-c6cccc(N(c7ccccc7)c7ccc8c(c7)c7ccccc7n8-c7ccccc7)c6)cc6ccccc6c5c4c3o2)cc1]

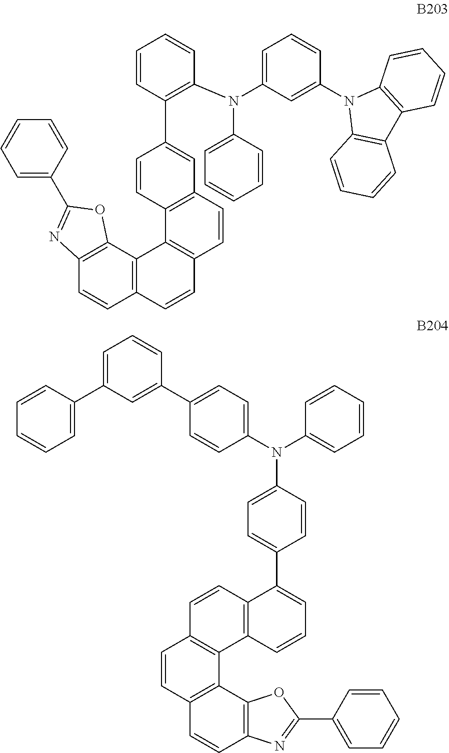

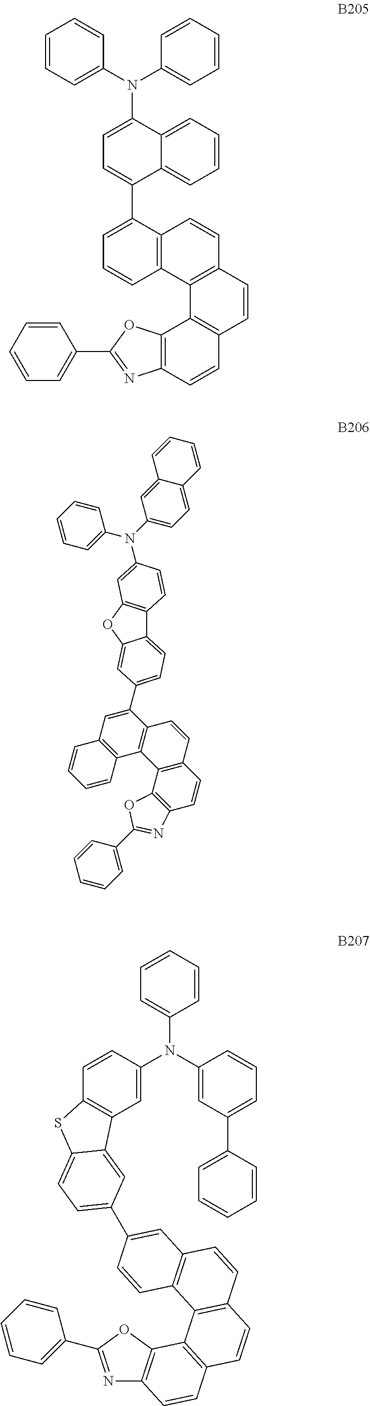 [SMILES: c1ccc(-c2nc3ccc4ccc5ccc6c(-c7ccc(N(c8ccccc8)c8ccccc8)c8ccccc78)cccc6c5c4c3o2)cc1 ; c1ccc(-c2nc3ccc4ccc5c(-c6ccc7c(c6)oc6cc(N(c8ccccc8)c8ccc9ccccc9c8)ccc67)cc6ccccc6c5c4c3o2)cc1 ; c1ccc(-c2cccc(N(c3ccccc3)c3ccc4sc5ccc(-c6ccc7c(ccc8ccc9ccc%10nc(-c%11ccccc%11)oc%10c9c87)c6)cc5c4c3)c2)cc1]

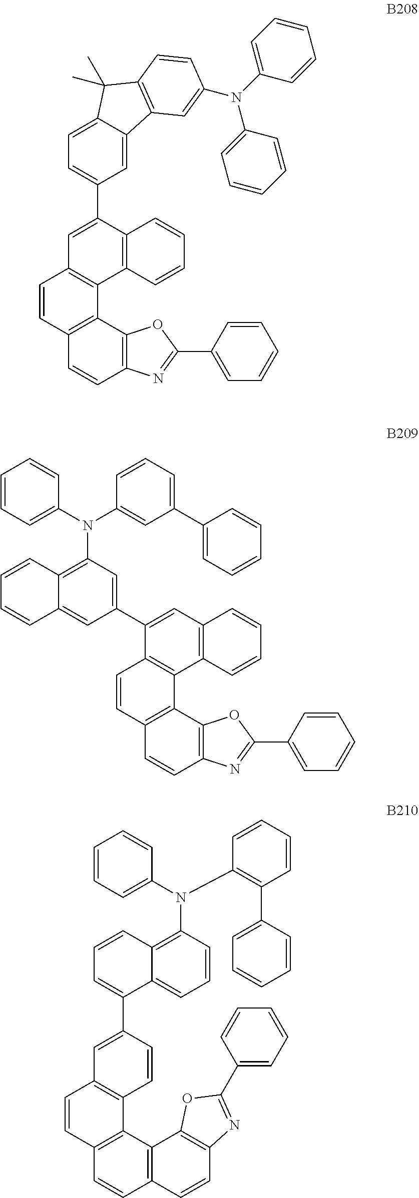 [SMILES: CC1(C)c2ccc(-c3cc4ccc5ccc6nc(-c7ccccc7)oc6c5c4c4ccccc34)cc2-c2cc(N(c3ccccc3)c3ccccc3)ccc21 ; c1ccc(-c2cccc(N(c3ccccc3)c3cc(-c4cc5ccccc5c5c4ccc4ccc6nc(-c7ccccc7)oc6c45)cc4ccccc34)c2)cc1 ; c1ccc(-c2nc3ccc4ccc5ccc6cc(-c7cccc8c(N(c9ccccc9)c9ccccc9-c9ccccc9)cccc78)ccc6c5c4c3o2)cc1]

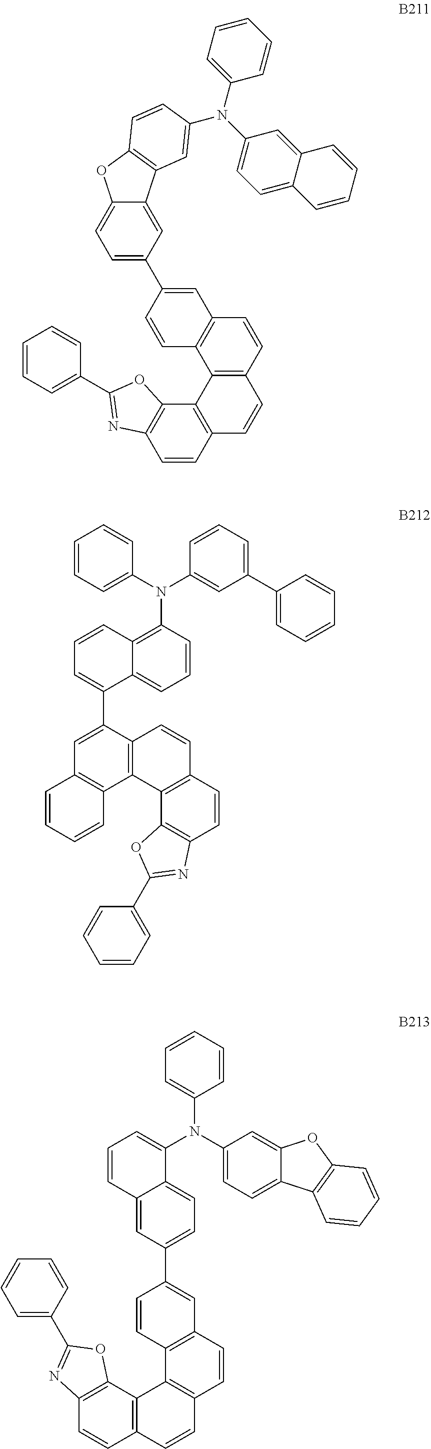 [SMILES: c1ccc(-c2nc3ccc4ccc5ccc6cc(-c7ccc8oc9ccc(N(c%10ccccc%10)c%10ccc%11ccccc%11c%10)cc9c8c7)ccc6c5c4c3o2)cc1 ; c1ccc(-c2cccc(N(c3ccccc3)c3cccc4c(-c5cc6ccccc6c6c5ccc5ccc7nc(-c8ccccc8)oc7c56)cccc34)c2)cc1 ; c1ccc(-c2nc3ccc4ccc5ccc6cc(-c7ccc8c(N(c9ccccc9)c9ccc%10c(c9)oc9ccccc9%10)cccc8c7)ccc6c5c4c3o2)cc1]

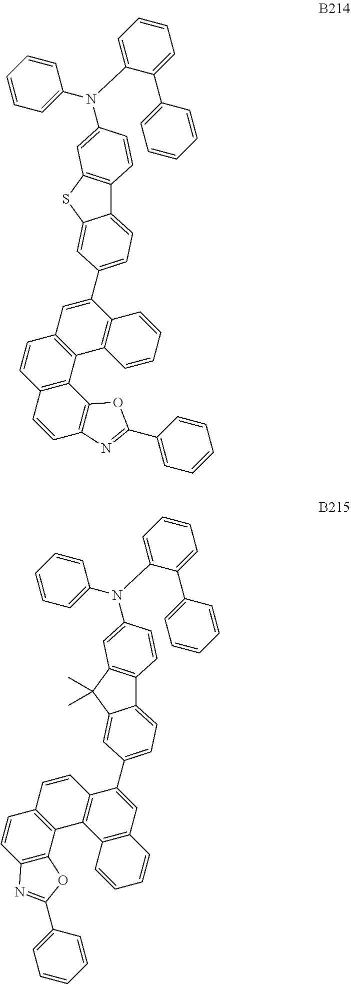 [SMILES: c1ccc(-c2nc3ccc4ccc5cc(-c6ccc7c(c6)sc6cc(N(c8ccccc8)c8ccccc8-c8ccccc8)ccc67)c6ccccc6c5c4c3o2)cc1 ; CC1(C)c2cc(-c3cc4ccccc4c4c3ccc3ccc5nc(-c6ccccc6)oc5c34)ccc2-c2ccc(N(c3ccccc3)c3ccccc3-c3ccccc3)cc21]

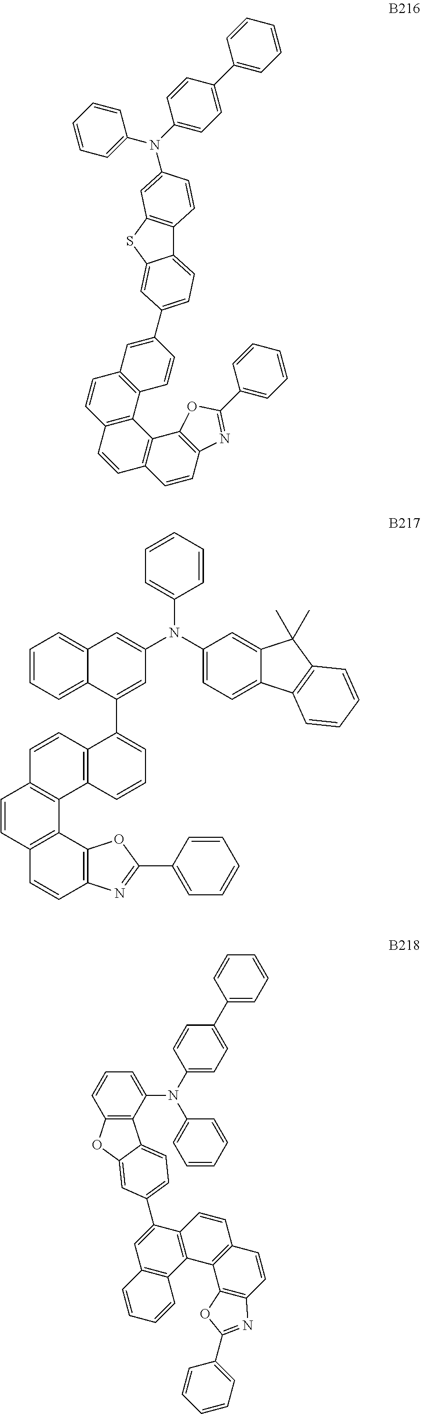 [SMILES: c1ccc(-c2ccc(N(c3ccccc3)c3ccc4c(c3)sc3cc(-c5ccc6c(ccc7ccc8ccc9nc(-c%10ccccc%10)oc9c8c76)c5)ccc34)cc2)cc1 ; CC1(C)c2ccccc2-c2ccc(N(c3ccccc3)c3cc(-c4cccc5c4ccc4ccc6ccc7nc(-c8ccccc8)oc7c6c45)c4ccccc4c3)cc21 ; c1ccc(-c2ccc(N(c3ccccc3)c3cccc4oc5cc(-c6cc7ccccc7c7c6ccc6ccc8nc(-c9ccccc9)oc8c67)ccc5c34)cc2)cc1]

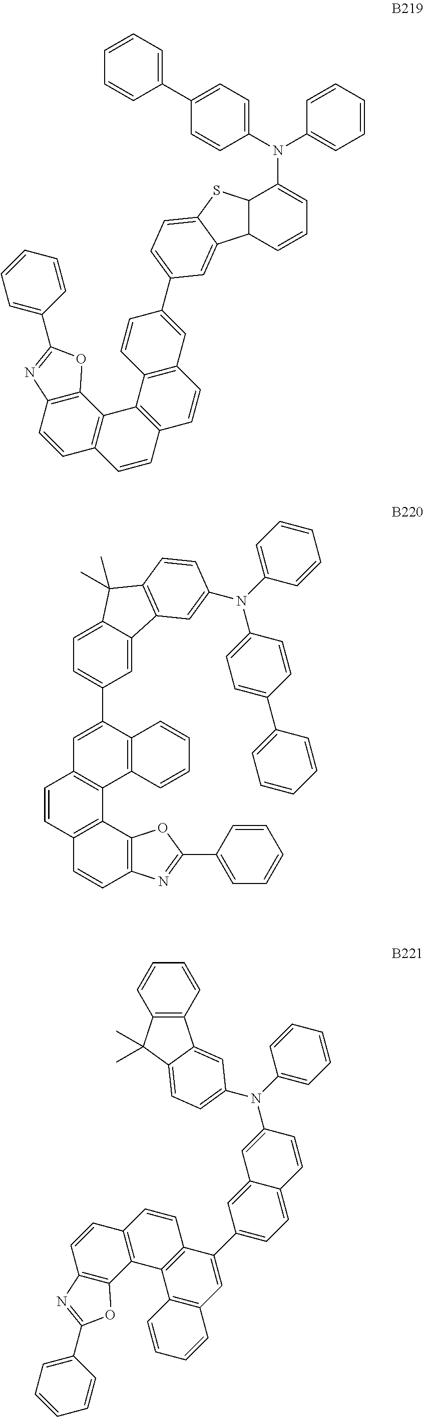 [SMILES: C1=CC2c3cc(-c4ccc5c(ccc6ccc7ccc8nc(-c9ccccc9)oc8c7c65)c4)ccc3SC2C(N(c2ccccc2)c2ccc(-c3ccccc3)cc2)=C1 ; CC1(C)c2ccc(-c3cc4ccc5ccc6nc(-c7ccccc7)oc6c5c4c4ccccc34)cc2-c2cc(N(c3ccccc3)c3ccc(-c4ccccc4)cc3)ccc21 ; CC1(C)c2ccccc2-c2cc(N(c3ccccc3)c3ccc4ccc(-c5cc6ccccc6c6c5ccc5ccc7nc(-c8ccccc8)oc7c56)cc4c3)ccc21]

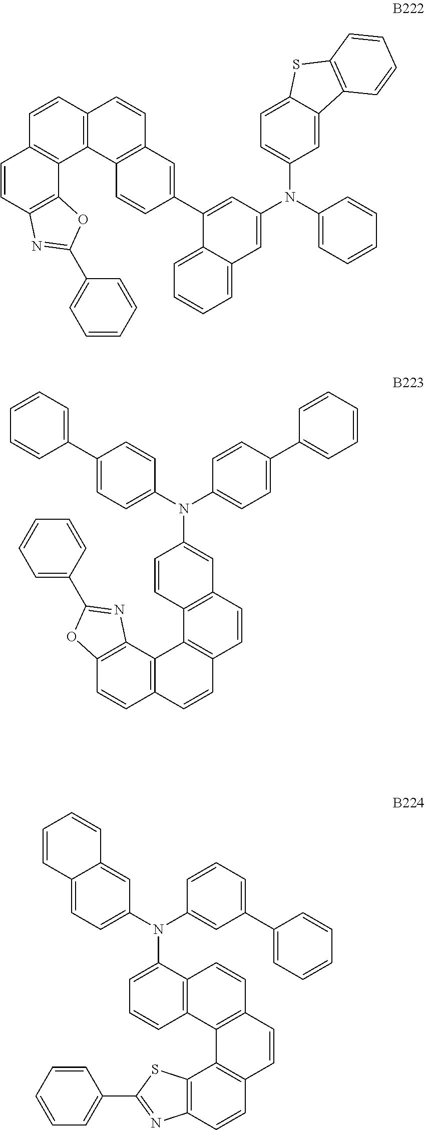 [SMILES: c1ccc(-c2nc3ccc4ccc5ccc6cc(-c7cc(N(c8ccccc8)c8ccc9sc%10ccccc%10c9c8)cc8ccccc78)ccc6c5c4c3o2)cc1 ; c1ccc(-c2ccc(N(c3ccc(-c4ccccc4)cc3)c3ccc4c(ccc5ccc6ccc7oc(-c8ccccc8)nc7c6c54)c3)cc2)cc1 ; c1ccc(-c2cccc(N(c3ccc4ccccc4c3)c3cccc4c3ccc3ccc5ccc6nc(-c7ccccc7)sc6c5c34)c2)cc1]

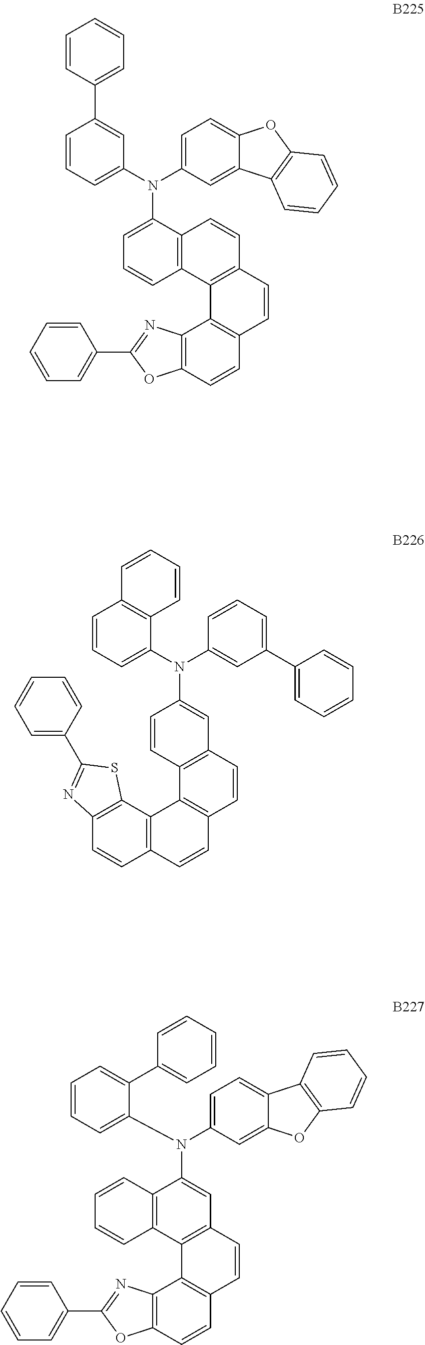 [SMILES: c1ccc(-c2cccc(N(c3ccc4oc5ccccc5c4c3)c3cccc4c3ccc3ccc5ccc6oc(-c7ccccc7)nc6c5c34)c2)cc1 ; c1ccc(-c2cccc(N(c3ccc4c(ccc5ccc6ccc7nc(-c8ccccc8)sc7c6c54)c3)c3cccc4ccccc34)c2)cc1 ; c1ccc(-c2nc3c(ccc4ccc5cc(N(c6ccc7c(c6)oc6ccccc67)c6ccccc6-c6ccccc6)c6ccccc6c5c43)o2)cc1]

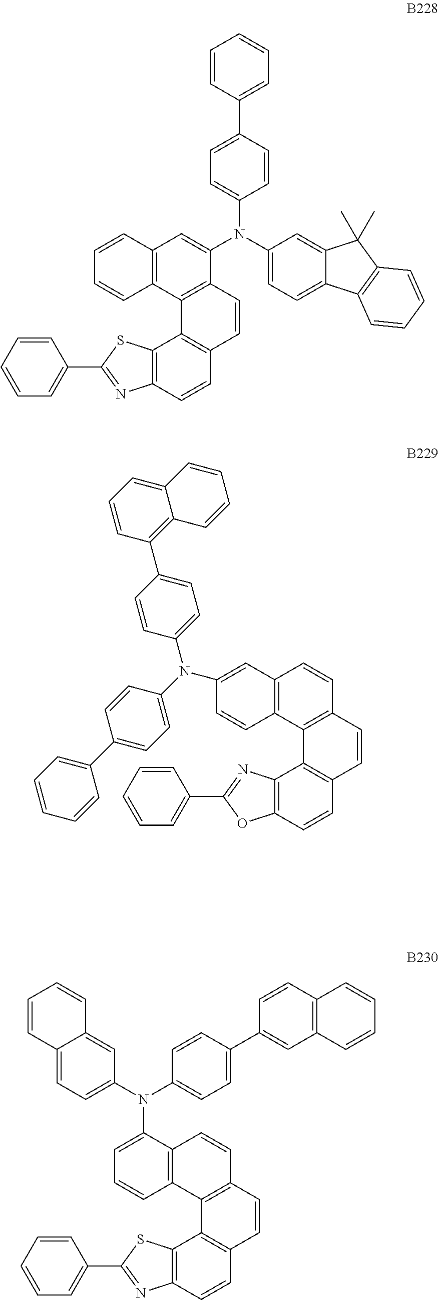 [SMILES: CC1(C)c2ccccc2-c2ccc(N(c3ccc(-c4ccccc4)cc3)c3cc4ccccc4c4c3ccc3ccc5nc(-c6ccccc6)sc5c34)cc21 ; c1ccc(-c2ccc(N(c3ccc(-c4cccc5ccccc45)cc3)c3ccc4c(ccc5ccc6ccc7oc(-c8ccccc8)nc7c6c54)c3)cc2)cc1 ; c1ccc(-c2nc3ccc4ccc5ccc6c(N(c7ccc(-c8ccc9ccccc9c8)cc7)c7ccc8ccccc8c7)cccc6c5c4c3s2)cc1]

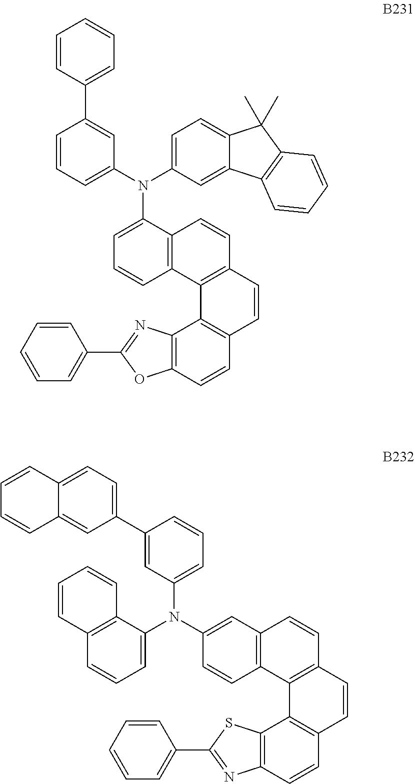 [SMILES: CC1(C)c2ccccc2-c2cc(N(c3cccc(-c4ccccc4)c3)c3cccc4c3ccc3ccc5ccc6oc(-c7ccccc7)nc6c5c34)ccc21 ; c1ccc(-c2nc3ccc4ccc5ccc6cc(N(c7cccc(-c8ccc9ccccc9c8)c7)c7cccc8ccccc78)ccc6c5c4c3s2)cc1]

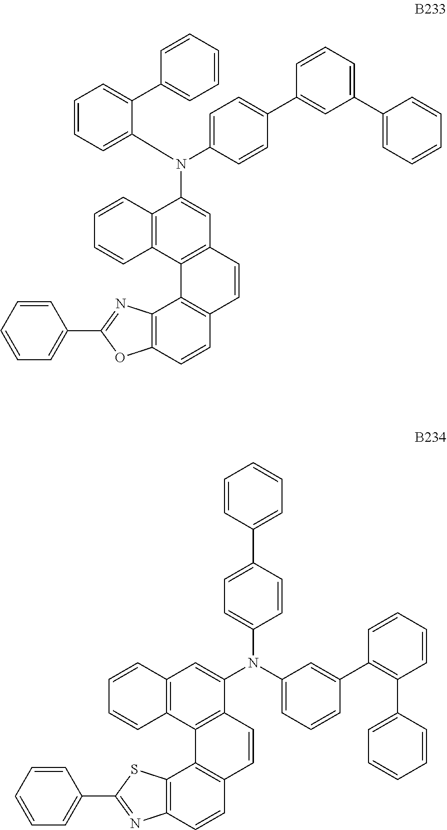 [SMILES: c1ccc(-c2cccc(-c3ccc(N(c4ccccc4-c4ccccc4)c4cc5ccc6ccc7oc(-c8ccccc8)nc7c6c5c5ccccc45)cc3)c2)cc1 ; c1ccc(-c2ccc(N(c3cccc(-c4ccccc4-c4ccccc4)c3)c3cc4ccccc4c4c3ccc3ccc5nc(-c6ccccc6)sc5c34)cc2)cc1]

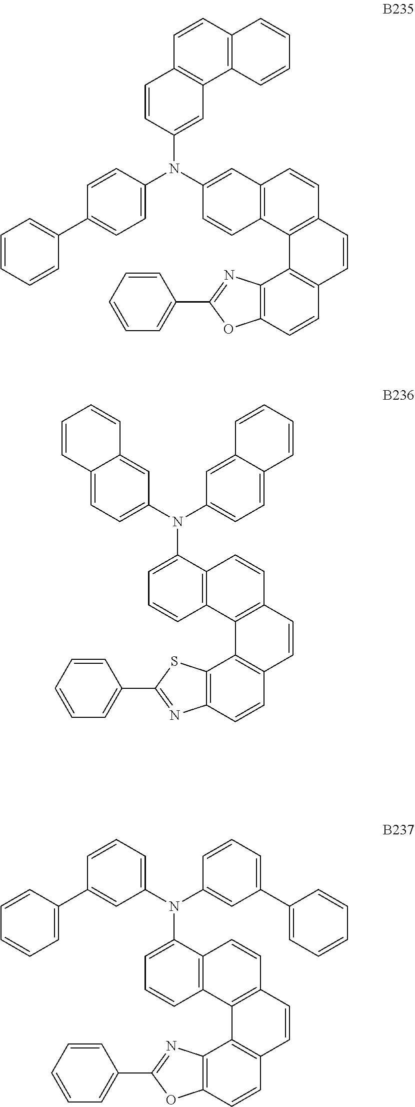 [SMILES: c1ccc(-c2ccc(N(c3ccc4c(ccc5ccc6ccc7oc(-c8ccccc8)nc7c6c54)c3)c3ccc4ccc5ccccc5c4c3)cc2)cc1 ; c1ccc(-c2nc3ccc4ccc5ccc6c(N(c7ccc8ccccc8c7)c7ccc8ccccc8c7)cccc6c5c4c3s2)cc1 ; c1ccc(-c2cccc(N(c3cccc(-c4ccccc4)c3)c3cccc4c3ccc3ccc5ccc6oc(-c7ccccc7)nc6c5c34)c2)cc1]

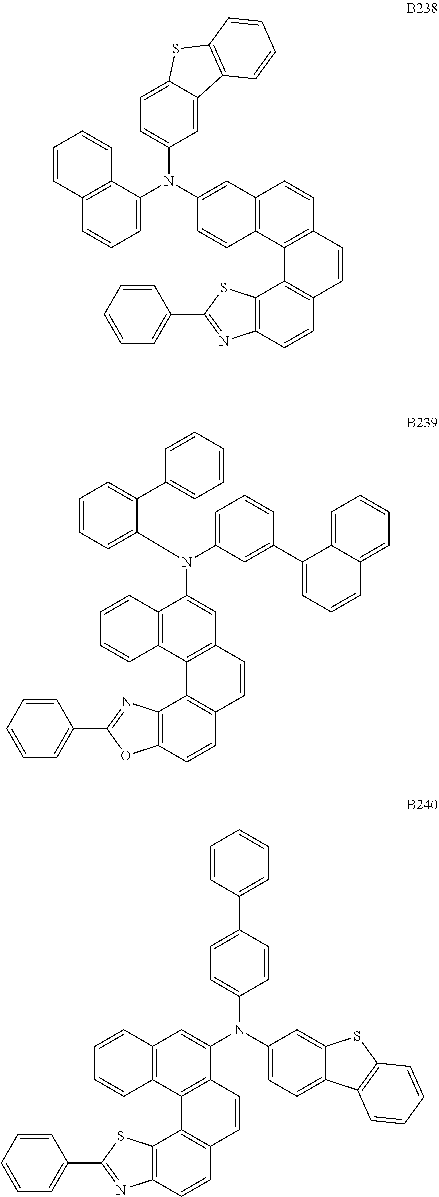 [SMILES: c1ccc(-c2nc3ccc4ccc5ccc6cc(N(c7ccc8sc9ccccc9c8c7)c7cccc8ccccc78)ccc6c5c4c3s2)cc1 ; c1ccc(-c2nc3c(ccc4ccc5cc(N(c6cccc(-c7cccc8ccccc78)c6)c6ccccc6-c6ccccc6)c6ccccc6c5c43)o2)cc1 ; c1ccc(-c2ccc(N(c3ccc4c(c3)sc3ccccc34)c3cc4ccccc4c4c3ccc3ccc5nc(-c6ccccc6)sc5c34)cc2)cc1]

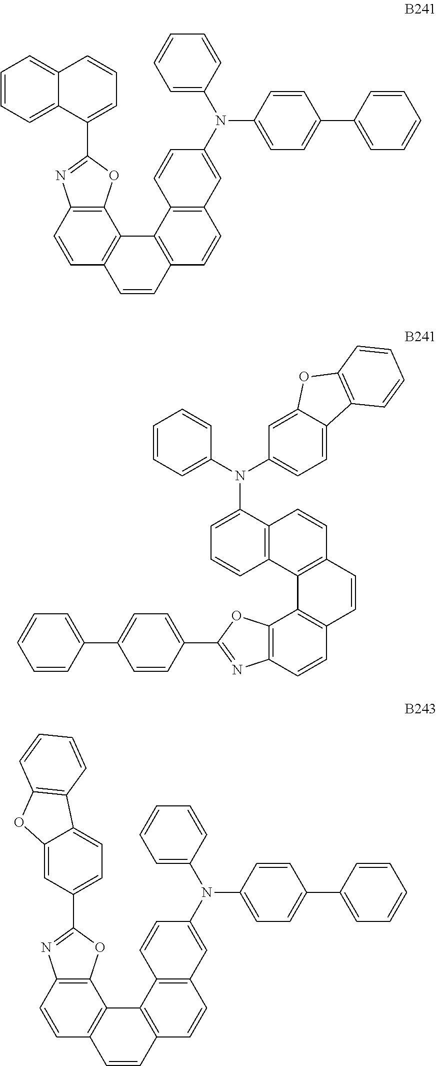 [SMILES: c1ccc(-c2ccc(N(c3ccccc3)c3ccc4c(ccc5ccc6ccc7nc(-c8cccc9ccccc89)oc7c6c54)c3)cc2)cc1 ; c1ccc(-c2ccc(-c3nc4ccc5ccc6ccc7c(N(c8ccccc8)c8ccc9c(c8)oc8ccccc89)cccc7c6c5c4o3)cc2)cc1 ; c1ccc(-c2ccc(N(c3ccccc3)c3ccc4c(ccc5ccc6ccc7nc(-c8ccc9c(c8)oc8ccccc89)oc7c6c54)c3)cc2)cc1]

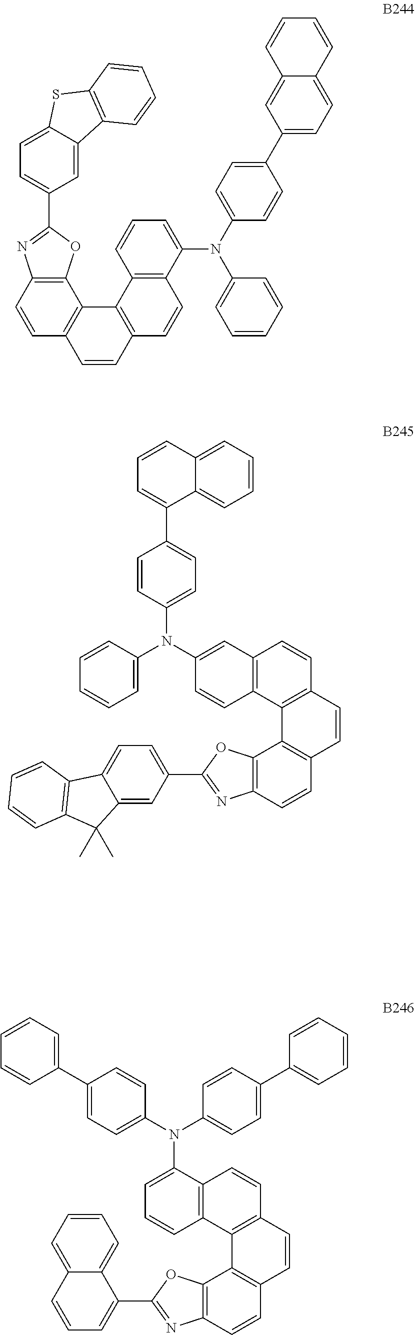 [SMILES: c1ccc(N(c2ccc(-c3ccc4ccccc4c3)cc2)c2cccc3c2ccc2ccc4ccc5nc(-c6ccc7sc8ccccc8c7c6)oc5c4c23)cc1 ; CC1(C)c2ccccc2-c2ccc(-c3nc4ccc5ccc6ccc7cc(N(c8ccccc8)c8ccc(-c9cccc%10ccccc9%10)cc8)ccc7c6c5c4o3)cc21 ; c1ccc(-c2ccc(N(c3ccc(-c4ccccc4)cc3)c3cccc4c3ccc3ccc5ccc6nc(-c7cccc8ccccc78)oc6c5c34)cc2)cc1]

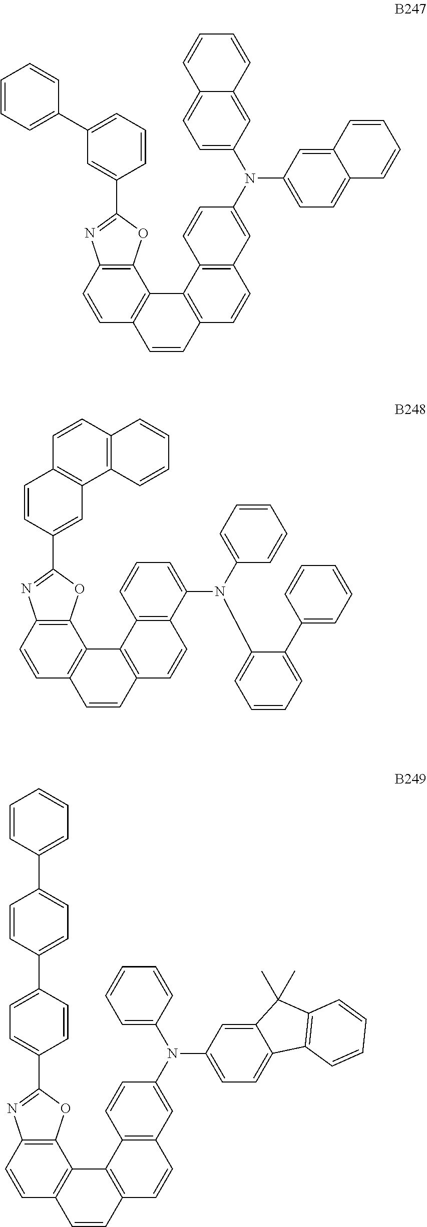 [SMILES: c1ccc(-c2cccc(-c3nc4ccc5ccc6ccc7cc(N(c8ccc9ccccc9c8)c8ccc9ccccc9c8)ccc7c6c5c4o3)c2)cc1 ; c1ccc(-c2ccccc2N(c2ccccc2)c2cccc3c2ccc2ccc4ccc5nc(-c6ccc7ccc8ccccc8c7c6)oc5c4c23)cc1 ; CC1(C)c2ccccc2-c2ccc(N(c3ccccc3)c3ccc4c(ccc5ccc6ccc7nc(-c8ccc(-c9ccc(-c%10ccccc%10)cc9)cc8)oc7c6c54)c3)cc21]

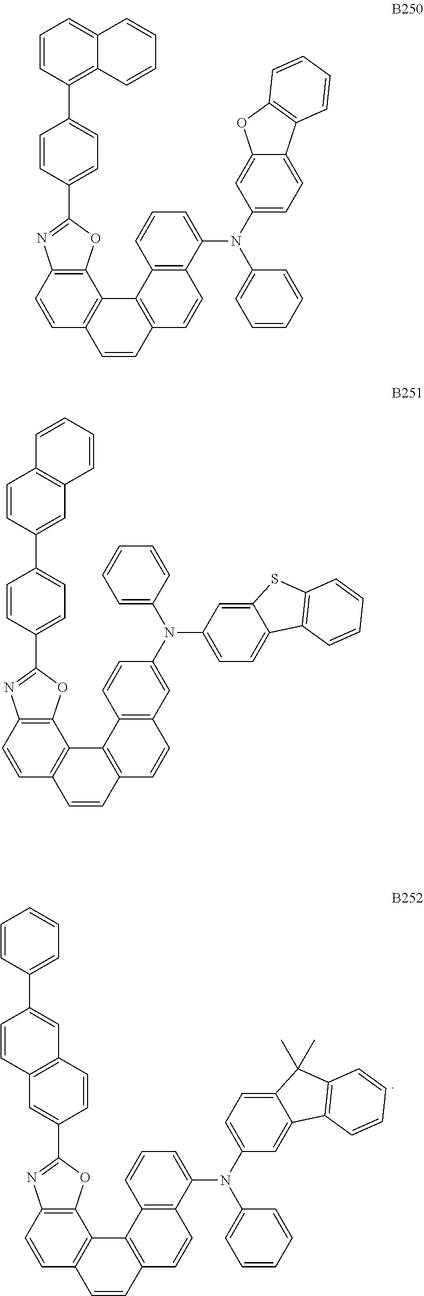 [SMILES: c1ccc(N(c2ccc3c(c2)oc2ccccc23)c2cccc3c2ccc2ccc4ccc5nc(-c6ccc(-c7cccc8ccccc78)cc6)oc5c4c23)cc1 ; c1ccc(N(c2ccc3c(ccc4ccc5ccc6nc(-c7ccc(-c8ccc9ccccc9c8)cc7)oc6c5c43)c2)c2ccc3c(c2)sc2ccccc23)cc1 ; CC1(C)c2ccccc2-c2cc(N(c3ccccc3)c3cccc4c3ccc3ccc5ccc6nc(-c7ccc8cc(-c9ccccc9)ccc8c7)oc6c5c34)ccc21]

15. An organic electroluminescent device, comprising an anode and a cathode disposed opposite to each other, and a functional layer disposed between the anode and the cathode, wherein the functional layer comprises the organic compound of claim 1;
optionally, the functional layer comprises an organic light-emitting layer, and the organic light-emitting layer comprises the nitrogen-containing compound.

16. An electronic apparatus, comprising the organic electroluminescent device of claim 15.
